(19) United States
(12) Patent Application Publication
(10) Pub. No.: US 2026/0002151 A1
(43) Pub. Date: Jan. 1, 2026

(54) COMPOSITIONS AND METHODS FOR THE MODIFICATION AND REGULATION OF LIVER GENE EXPRESSION

(71) Applicant: Mammoth Biosciences, Inc., Brisbane, CA (US)

(72) Inventors: Raymond D. HICKEY, Belmont, CA (US); Sean CHEN, Brisbane, CA (US); Ymer Leif Michael BJORNSON, Gilroy, CA (US); Benjamin Julius RAUCH, San Francisco, CA (US); William Douglass WRIGHT, Fairfield, CA (US); Stepan TYMOSHENKO, Sacramento, CA (US); Wiputra Jaya HARTONO, San Francisco, CA (US); Aaron DELOUGHERY, San Francisco, CA (US); Lucas Benjamin HARRINGTON, San Francisco, CA (US); Lauren Kelli UYESAKA, San Francisco, CA (US)

(73) Assignee: Mammoth Biosciences, Inc., Brisbane, CA (US)

(21) Appl. No.: 19/309,745

(22) Filed: Aug. 26, 2025

Related U.S. Application Data
(63) Continuation of application No. PCT/US2024/017553, filed on Feb. 27, 2024.
(60) Provisional application No. 63/487,259, filed on Feb. 27, 2023.
(60) Provisional application No. 63/487,258, filed on Feb. 27, 2023.
(60) Provisional application No. 63/515,084, filed on Jul. 21, 2023.
(60) Provisional application No. 63/586,918, filed on Sep. 29, 2023.
(60) Provisional application No. 63/616,929, filed on Jan. 2, 2024.

(51) Int. Cl. C12N 15/11; A61P 5/00; C12N 9/22; C12N 9/78; C12N 15/86; C12N 15/88

(52) U.S. Cl. CPC C12N 15/11; A61P 5/00; C12N 9/222; C12N 9/78; C12N 15/86; C12N 15/88; C12Y 305/04002; C07K 2319/00; C12N 2310/20; C12N 2750/14143

(57) ABSTRACT

Provided herein are compositions, systems, and methods for modifying a human APOC3 gene, PCSK9 gene, or ANGPTL3 gene. Systems, compositions, and methods may comprise a CRISPR-associated (Cas) protein or uses thereof. Systems, compositions, and methods of the present disclosure may be useful for treatment of APOC3 associated conditions, including familial chylomicronemia syndrome (FCS) and severe hypertriglyceridemia (SHTG).

### CROSS-REFERENCE TO RELATED APPLICATIONS

The present application is a continuation of International PCT Application No. PCT/US2024/017553, filed Feb. 27, 2024, which claims priority to U.S. Provisional Application 63/487,258, filed Feb. 27, 2023; U.S. Provisional Application 63/487,259, filed Feb. 27, 2023; U.S. Provisional Application 63/515,084, filed Jul. 21, 2023; U.S. Provisional Application 63/586,918, filed Sep. 29, 2023; U.S. Provisional Application 63/616,929, filed Jan. 2, 2024, the contents each of which are incorporated herein by reference in their entireties.

### SEQUENCE LISTING

The contents of the electronic sequence listing (MABI_031_04US_SeqList_ST26.xml; Size: 1,953,769 bytes; and Date of Creation: Jun. 13, 2025) are herein incorporated by reference in its entirety.

### BACKGROUND

Apolipoprotein C3 (APOC3) is a key regulator of plasma triglyceride levels. APOC3 is secreted in the liver and small intestine. APOC3 regulates liver uptake of triglyceride-rich lipoproteins through lipoprotein lipase (LPL)-dependent and LPL-independent mechanisms. It has been suggested that APOC3 may exert pro-atherogenic effects directly by enhancing vessel wall inflammation and indirectly by promoting hypertriglyceridemia. Individuals with loss of function mutations in APOC3 show ˜40% reduction in both triglyceride levels and risk for atherosclerotic cardiovascular disease (ASCVD) compared with non-carriers. Furthermore, epidemiological studies have concluded that APOC3 levels predict risk of ASCVD and cardiovascular mortality.

Familial chylomicronemia syndrome (FCS) is a rare autosomal recessive disease characterized by the buildup in the blood of fat particles called chylomicrons (chylomicronemia), severe hypertriglyceridemia, and the risk of recurrent and potentially fatal pancreatitis and other complications. It is caused by mutations in the gene encoding LPL or, less frequently, by mutations in genes encoding other proteins necessary for LPL function. People with FCS are at high risk of unpredictable and potentially fatal acute pancreatitis. In addition to pancreatitis, FCS patients are at risk of chronic complications due to permanent organ damage, including chronic pancreatitis and pancreatogenic (Type 3c) diabetes. They can experience daily symptoms including abdominal pain, generalized fatigue and impaired cognition that affect their ability to work. People with FCS also report major emotional and psychosocial effects including anxiety, social withdrawal, depression, and brain fog.

Severe hypertriglyceridemia (SHTG) is a common condition characterized by high levels of triglycerides in the bloodstream. SHTG (triglyceride levels ≥500 mg/dL) can be caused by diet-derived chylomicronemia and excessive liver triglyceride production, often superimposed on genetic predisposition. Its primary manifestation is acute pancreatitis, particularly if triglyceride levels are >880 mg/dL. A subset of patients with triglyceride levels 500-880 are also at risk for cardiovascular disease. Lowering of plasma triglycerides is desired. Hypertriglyceridemia can lead to conditions including atherosclerosis (hardening of the arteries), obesity, and insulin resistance, which all can contribute to increased risk of cardiovascular disease. SHTG is also a known risk factor for acute pancreatitis, a life-threatening condition.

Another regulator of plasma triglyceride levels is proprotein convertase subtilisin kexin type 9 (PCSK9). PCSK9 binds to, and degrades, the receptor for low-density lipoprotein particles (LDL). The LDL receptor (LDLR), on liver and other cell membranes, binds and initiates ingestion of LDL-particles from extracellular fluid into cells and targets the complex to lysosomes for destruction. If PCSK9 is blocked or degraded, the LDL-LDLR complex separates during trafficking, with the LDL digested in the lysosome, but the LDLRs instead recycled back to the cell surface and so able to remove additional LDL-particles from the extracellular fluid. Therefore, agents that reduce PCSK9 may lower LDL particle concentrations.

A third regulator of plasma triglyceride levels is Angiopoietin-like 3 (ANGPTL3). ANGPTL3 acts as a dual inhibitor of lipoprotein lipase and endothelial lipase thereby increasing plasma triglyceride, LDL cholesterol and HDL cholesterol in mice and humans. Therefore, agents that reduce ANGPTL3 may lower LDL particle concentrations.

### SUMMARY

The present disclosure provides systems and compositions for modifying APOC3, PCSK9, and ANGPTL3, and uses thereof. Such systems and compositions generally comprise guide nucleic acids and CRISPR associated (Cas) proteins to reduce or abolish expression of the APOC3, PCSK9, or ANGPTL3 protein. Compositions, systems, and methods disclosed herein may leverage nucleic acid modifying activities. Nucleic acid modifying activities may include, by way of non-limiting example, cis cleavage activity, nickase activity, and nucleobase modifying activity.

In some aspects, disclosed herein is a composition or system comprising a guide ribonucleic acid (RNA) or a polynucleotide encoding the same, wherein the guide RNA comprises (a) a first region comprising a protein binding sequence, and (b) a second region comprising a targeting sequence that is complementary to a target sequence that is within an APOC3 gene, wherein the targeting sequence comprises a nucleotide sequence that is at least 70%, at least 75%, at least 80%, at least 85%, at least 90%, at least 95% or 100% identical to any one of SEQ ID NOs: 1-15, 67-72, 207, 209-299, 804-805, 823-825, 830-1399, 2018-2026, and 2084-2086. In some embodiments, the targeting sequence is selected from SEQ ID NOs: 1-15, 67-72, 207, 209-299, 804-805, 823-825, 830-1399, 2018-2026, and 2084-2086. In some embodiments, the targeting sequence comprises a nucleotide sequence that is at least 70%, at least 75%, at least 80%, at least 85%, at least 90%, at least 95% or 100% identical to any one of SEQ ID NOs: 1-15, 67-72, 207, 804-805, and 830-999, and the protein binding sequence comprises a nucleotide sequence that is at least 70%, at least 75%, at least 80%, at least 85%, at least 90%, at least 95% or 100% identical to any one of SEQ ID NOs: 16 and 38-43. In some embodiments, the composition or system comprises an effector protein or a nucleic acid encoding the same, wherein the effector protein comprises an amino acid sequence that is at least 70%, at least 75%, at least 80%, at least 85%, at least 90%, at least 95%, at least 97%, at least 98%, at least 99%, or 100% identical to any one of SEQ ID NOs: 32, 34, 794, or 2090. In some embodiments, the effector protein comprises an amino acid alteration relative to SEQ ID NO: 32 as described in TABLE 18 or TABLE 19.

In some embodiments, the guide RNA comprises a nucleotide sequence that is at least 70%, at least 75%, at least 80%, at least 85%, at least 90%, at least 95% or 100% identical to any one of SEQ ID NOs: 17-31, 73-78, 491, 815-816, and 1400-1569. In some embodiments, the targeting sequence comprises a nucleotide sequence that is at least 70%, at least 75%, at least 80%, at least 85%, at least 90%, at least 95% or 100% identical to any one of SEQ ID NOs: 209-299, 823-825, 1000-1399, 2018-2026, and 2084-2086, and the protein binding sequence comprises a nucleotide sequence that is at least 70%, at least 75%, at least 80%, at least 85%, at least 90%, at least 95% or 100% identical to SEQ ID NO: 488. In some embodiments, the protein binding sequence further comprises a nucleotide sequence that is at least 70%, at least 75%, at least 80%, at least 85%, at least 90%, at least 95% or 100% identical to SEQ ID NOs: 489 or 490. In some embodiments, the composition or system comprises an effector protein or a nucleic acid encoding the same, wherein the effector protein comprises an amino acid sequence that is at least 70%, at least 75%, at least 80%, at least 85%, at least 90%, at least 95%, at least 97%, at least 98%, at least 99%, or 100% identical to any one of SEQ ID NOs: 773, 775, or 793. In some embodiments, the effector protein comprises an amino acid alteration relative to SEQ ID NO: 773 as described in TABLE 16 or TABLE 17. In some embodiments, the guide RNA comprises a nucleotide sequence that is at least 70%, at least 75%, at least 80%, at least 85%, at least 90%, at least 95%, or 100% identical to any one of SEQ ID NOs: 494-584, 826-828, 1570-1969, 2075-2083, and 2087-2089.

In some embodiments, the first region comprises a nucleotide sequence that is at least 70%, at least 75%, at least 80%, at least 85%, at least 90%, at least 95%, or 100% identical to SEQ ID NO: 39, and a second region comprising a nucleotide sequence that is at least 70%, at least 75%, at least 80%, at least 85%, at least 90%, at least 95%, or 100% identical to SEQ ID NO: 10. In some embodiments, the guide RNA comprises a nucleotide sequence that is at least 70%, at least 75%, at least 80%, at least 85%, at least 90%, at least 95%, or 100% identical to SEQ ID NO: 26.

In some embodiments, the first region comprises a nucleotide sequence that is at least 70%, at least 75%, at least 80%, at least 85%, at least 90%, at least 95%, or 100% identical to SEQ ID NO: 39, and a second region comprising a nucleotide sequence that is at least 70%, at least 75%, at least 80%, at least 85%, at least 90%, at least 95%, or 100% identical to SEQ ID NO: 71. In some embodiments, the guide RNA comprises a nucleotide that is at least 70%, at least 75%, at least 80%, at least 85%, at least 90%, at least 95%, or 100% identical to SEQ ID NO: 77. In some embodiments, the composition or system further comprises an effector protein or a nucleic acid encoding the same, wherein the effector protein comprises an amino acid sequence at least 70%, at least 75%, at least 80%, at least 85%, at least 90%, at least 95%, or 100% identical to any one of SEQ ID NOs: 32, 34, 794, or 2090.

In some embodiments, the nucleic acid encoding the effector protein comprises a messenger RNA. In some embodiments, the effector protein is fused to a fusion partner protein or wherein the nucleic acid encoding the effector protein encodes a fusion partner protein that is fused to the effector protein upon expression of the nucleic acid. In some embodiments, the fusion partner protein comprises an enzymatic activity is selected from reverse transcriptase activity, deaminase activity, and methyltransferase activity. In some embodiments, the composition or system further comprises a lipid nanoparticle (LNP), wherein the LNP contains the guide nucleic acid, and optionally, the effector protein or nucleic acid encoding the same.

In some aspects, disclosed herein is a composition or system comprising an expression cassette comprising, from 5′ to 3′: (a) a first inverted terminal repeat (ITR); (b) a first promoter sequence operably linked to a nucleic acid sequence encoding a guide RNA wherein the guide RNA comprises: (i) a first region comprising a protein binding sequence; and (ii) a second region comprising a spacer sequence that is complementary to a target sequence of an APOC3 gene, wherein the spacer sequence is at least 70%, at least 75%, at least 80%, at least 85%, at least 90%, at least 95%, or 100% identical to any one of SEQ ID NOs: 1-15, 67-72, 207, 209-299, 804-805, 823-825, 830-1399, 2018-2026, and 2084-2086; (c) a second promoter sequence operably linked to a nucleic acid sequence encoding an effector protein, wherein the effector protein comprises an amino acid sequence that is at least 70%, at least 75%, at least 80%, at least 85%, at least 90%, at least 95%, or 100% identical to an amino acid sequence selected from SEQ ID NOs: 32 and 773; (d) a poly(A) signal; and (e) a second ITR. In some embodiments, the expression cassette is an adeno-associated virus (AAV) vector or portion thereof.

In some aspects, disclosed herein is a pharmaceutical composition comprising the composition of any one of the above aspects or embodiments, and a pharmaceutical acceptable excipient or carrier.

In some aspects, disclosed herein is method of modifying an APOC3 gene, comprising contacting the APOC3 gene, with the composition or system of any one of the above aspects or embodiments. In some embodiments, modifying the APOC3 gene reduces the expression of the APOC3 gene. In some embodiments, modifying the APOC3 gene permanently reduces the expression of the APOC3 gene. In some embodiments, modifying the APOC3 gene comprises cleaving at least one strand of the APOC3 gene. In some embodiments, modifying the APOC3 gene is in vivo. In some embodiments, modifying the APOC3 gene is in the liver.

In some aspects, disclosed herein is a method of lowering triglycerides in a mammal with hypertriglyceridemia, the method comprising delivering a composition to the mammal, wherein the composition comprises: (a) a guide nucleic acid comprising a nucleotide sequence that is at least 70%, at least 75%, at least 80%, at least 85%, at least 90%, at least 95%, or 100% identical to a nucleotide sequence selected from any one of SEQ ID NOs: 1-31, 38-43, 67-202, 207-772, 779-820, and 820-2089 and (b) an effector protein or nucleic acid encoding the same, wherein the effector protein comprises an amino acid sequence that is at least 70%, at least 75%, at least 80%, at least 85%, at least 90%, at least 95%, or 100% identical to a nucleotide sequence selected from any one of SEQ ID NOs: 32 and 773. In some embodiments, the guide nucleic acid and the effector protein or nucleic acid encoding the same are delivered in an LNP.

In some aspects, disclosed herein is a method of treating or preventing a disease in a subject in need thereof, comprising administering the composition or system of any one of the above aspects or embodiments. In some embodiments, the disease is selected from cardiovascular disease, familial chylomicronemia syndrome, and hypertriglyceridemia.

In some aspects, disclosed herein is a cell, or population of cells, comprising, or modified by, the composition, system, or method of any one of the above aspects or embodiments. In some embodiments, the cell is a human cell.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

FIG. 1 shows editing of APOC3 in a human liver cell line as measured by % indel with CasPhi.12 L26R and various guide nucleic acids comprising a spacer sequence complementary to a target sequence in APOC3.

FIG. 2 shows editing of APOC3 in a human liver cell line as measured by % indel (left column) and reduction of APOC3 protein (right column) by CasPhi.12 L26R or CasM.265466 D220R and various guide nucleic acids comprising a spacer sequence complementary to a target sequence in APOC3.

FIG. 3A-FIG. 3C show editing of APOC3 with CasPhi.12 L26R in primary monkey hepatocytes from three different donors: Donor 1 (FIG. 3A), Donor 2 (FIG. 3B), and Donor 3 (FIG. 3C). For each guide, the column on the left is the percent indel formation with 200 ng of the guide RNA and the column on right is the percent indel formation with 50 ng of the guide RNA.

FIG. 4 shows editing of APOC3 with CasPhi.12 L26R and CasM.265466 D220R in primary monkey hepatocytes.

FIG. 5A-FIG. 5B show editing of APOC3 in a human liver cell line as measured by % indel with CasPhi.12 L26R or CasM.265466 and various guide nucleic acids comprising a spacer sequence complementary to a target sequence in APOC3. Guides R15579 and R15578 were paired with SpyCas9; guides R17561, R17562, R17563, R17564, R17565, R17566, R15592, and R15595 were paired with CasPhi.12; and the rest of the guides were paired with CasM.265466.

FIG. 6 shows editing of APOC3 and reduction of APOC3 protein in a human liver cell line as measured by % indel with CasPhi.12 L26R or CasM.265466 and various guide nucleic acids comprising a spacer sequence complementary to a target sequence in APOC3. Guide R15579 was paired with SpyCas9; guides R15592, R15595, R17561, R17562, R17563, R17564, R17566, and R17567 were paired with CasPhi.12; and the rest of the guides were paired with CasM.265466.

FIG. 7 shows that CasPhi.12 L26R can edit APOC3 across multiple NHP and human cell lines, wherein lighter color in the grey-scale heat map is indicative of indel formation.

FIG. 8 shows CasPhi.12 and CasM.265466 edit APOC3 in fibroblasts of hAPOC3 transgenic mice. Guide R15579 was paired with SpyCas9; guides R15592, R15595, R17561, R17562, R17563, R17566, and R17567 were paired with CasPhi.12; and the rest of the guides were paired with CasM.265466.

FIG. 9 shows that an mRNA encoding a CasPhi.12 variant can be delivered to a mouse via LNP can edit a gene in liver.

FIG. 10A shows that a CasM.265466 D220R/E335Q deaminase fusion protein can modify a nucleobase of APOC3, PCSK9, and ANGPTL3.

FIG. 10B shows that a CasPhi.12 L26R/E567Q deaminase fusion protein can modify a nucleobase of APOC3, PCSK9, and ANGPTL3. The bar to the left represents mean non-target strand ABE editing percent, and the bar to the right represents mean target position editing.

FIG. 11 shows that CasPhi.12 L26R and CasM.265466 D220R reduce human APOC3 protein in the livers of humanized APOC3 mice with severe hypertriglyceridemia and hypercholesterolemia.

FIG. 12 shows that CasPhi.12 L26R and CasM.265466 D220R reduce circulating triglycerides in humanized APOC3 mice with severe hypertriglyceridemia and hypercholesterolemia. The guide IDs shown in the legend from top to bottom correspond to the data points in the graphs from left to right.

FIG. 13 shows that CasPhi.12 variant L26R/I471T and various guide nucleic acids reduce human APOC3 protein in the livers of humanized APOC3 mice with severe hypertriglyceridemia and hypercholesterolemia.

FIG. 14A-FIG. 14D show that CasPhi.12 variant L26R/I471T reduces circulating triglycerides (FIG. 14B), LDL cholesterol (FIG. 14D), HDL cholesterol (FIG. 14C), and total cholesterol (FIG. 14A) in humanized APOC3 mice with severe hypertriglyceridemia and hypercholesterolemia. The guide IDs shown in the legend from top to bottom correspond to the data points in the graphs from left to right.

### DETAILED DESCRIPTION OF THE INVENTION

It is to be understood that both the foregoing general description and the following detailed description are exemplary, and explanatory only, and are not restrictive of the disclosure.

The section headings used herein are for organizational purposes only and are not to be construed as limiting the subject matter described.

All documents, or portions of documents, cited in this application, including, but not limited to, patents, patent applications, articles, books, and treatises, are hereby expressly incorporated by reference in their entirety for any purpose.

### 1. Definitions

Unless otherwise indicated, all technical terms used herein have the same meaning as commonly understood by one of ordinary skill in the art to which this invention belongs. Unless otherwise indicated or obvious from context, the following terms have the following meanings:

The terms, “a,” “an,” and “the,” as used herein, include plural references unless the context clearly dictates otherwise.

The terms, “or” and “and/or,” as used herein, include any, and all, combinations of one or more of the associated listed items.

The terms, “including,” “includes,” “included,” and other forms, are not limiting.

The terms, “comprise” and its grammatical equivalents, as used herein, specify the presence of stated features, integers, steps, operations, elements, and/or components, but do not preclude the presence or addition of one or more other features, integers, steps, operations, elements, components, and/or groups thereof.

The term, “about,” as used herein in reference to a number or range of numbers, is understood to mean the stated number and numbers+/−10% thereof, or 10% below the lower listed limit and 10% above the higher listed limit for the values listed for a range.

The terms, “% identical,” “% identity,” and “percent identity,” or grammatical equivalents thereof, refer to the extent to which two sequences (nucleotide or amino acid) have the same residue at the same positions in an alignment. For example, “an amino acid sequence is X % identical to SEQ ID NO: Y” can refer to % identity of the amino acid sequence to SEQ ID NO: Y and is elaborated as X % of residues in the amino acid sequence are identical to the residues of sequence disclosed in SEQ ID NO: Y. Generally, computer programs can be employed for such calculations. Illustrative programs that compare and align pairs of sequences, include ALIGN (Myers and Miller, Comput Appl Biosci. 1988 March; 4(1):11-7), FASTA (Pearson and Lipman, Proc Natl Acad Sci USA. 1988 April; 85(8):2444-8; Pearson, Methods Enzymol. 1990; 183:63-98) and gapped BLAST (Altschul et al., Nucleic Acids Res. 1997 Sep. 1; 25(17):3389-40), BLASTP, BLASTN, or GCG.

The term “base editing enzyme,” as used herein, refers to a protein, polypeptide or fragment thereof that is capable of catalyzing the chemical modification of a nucleobase of a deoxyribonucleotide or a ribonucleotide. Such a base editing enzyme, for example, is capable of catalyzing a reaction that modifies a nucleobase that is present in a nucleic acid molecule, such as DNA or RNA (single stranded or double stranded). Non-limiting examples of the type of modification that a base editing enzyme is capable of catalyzing includes converting an existing nucleobase to a different nucleobase, such as converting a cytosine to a guanine or thymine or converting an adenine to a guanine, hydrolytic deamination of an adenine or adenosine, or methylation of cytosine (e.g., CpG, CpA, CpT or CpC). A base editing enzyme itself may or may not bind to the nucleic acid molecule containing the nucleobase.

The term “base editor,” as used herein, refers to a fusion protein comprising a base editing enzyme linked to an effector protein. The base editing enzyme may be referred to as a fusion partner. The base editing enzyme can differ from a naturally occurring base editing enzyme. It is understood that any reference to a base editing enzyme herein also refers to a base editing enzyme variant. The base editor is functional when the effector protein is coupled to a guide nucleic acid. The guide nucleic acid imparts sequence specific activity to the base editor. By way of non-limiting example, the effector protein may comprise a catalytically inactive effector protein. Also, by way of non-limiting example, the base editing enzyme may comprise deaminase activity. Additional base editors are described herein.

The term “catalytically inactive effector protein,” also referred to as a “dCas” protein, as used herein, refers to an effector protein that is modified relative to a naturally-occurring effector protein to have a reduced or eliminated catalytic activity relative to that of the naturally-occurring effector protein, but retains its ability to interact with a guide nucleic acid. The catalytic activity that is reduced or eliminated is often a nuclease activity. The naturally-occurring effector protein may be a wildtype protein. In some embodiments, the catalytically inactive effector protein is referred to as a catalytically inactive variant of an effector protein, e.g., a Cas effector protein. In some embodiments, the catalytically inactive effector protein is referred to as a dead Cas protein or a dCas protein.

The term “cis cleavage,” as used herein, refers to cleavage (hydrolysis of a phosphodiester bond) of a target nucleic acid by an effector protein complexed with a guide nucleic acid (e.g., an RNP complex), wherein at least a portion of the guide nucleic acid is hybridized to at least a portion of the target nucleic acid. Cleavage may occur within or directly adjacent to the region of the target nucleic acid that is hybridized to the guide nucleic acid.

The terms “complementary” and “complementarity,” as used herein, with reference to a nucleic acid molecule or nucleotide sequence, refer to the characteristic of a polynucleotide having nucleotides that base pair with their Watson-Crick counterparts (C with G; or A with T or U) in a reference nucleic acid. For example, when every nucleotide in a polynucleotide forms a base pair with a reference nucleic acid, that polynucleotide is said to be 100% complementary to the reference nucleic acid. In a double stranded DNA or RNA sequence, the upper (sense) strand sequence is in general, understood as going in the direction from its 5′- to 3′-end, and the complementary sequence is thus understood as the sequence of the lower (antisense) strand in the same direction as the upper strand. Following the same logic, the reverse sequence is understood as the sequence of the upper strand in the direction from its 3′- to its 5′-end, while the ‘reverse complement’ sequence or the ‘reverse complementary’ sequence is understood as the sequence of the lower strand in the direction of its 5′- to its 3′-end. Each nucleotide in a double stranded DNA or RNA molecule that is paired with its Watson-Crick counterpart called its complementary nucleotide.

The term “cleavage assay,” as used herein, refers to an assay designed to visualize, quantitate, or identify cleavage of a nucleic acid. In some cases, the cleavage activity may be cis-cleavage activity. In some cases, the cleavage activity may be trans-cleavage activity.

The terms “cleave,” “cleaving,” and “cleavage,” as used herein, with reference to a nucleic acid molecule or nuclease activity of an effector protein, refer to the hydrolysis of a phosphodiester bond of a nucleic acid molecule that results in breakage of that bond. The result of this breakage can be a nick (hydrolysis of a single phosphodiester bond on one side of a double-stranded molecule), single strand break (hydrolysis of a single phosphodiester bond on a single-stranded molecule) or double strand break (hydrolysis of two phosphodiester bonds on both sides of a double-stranded molecule) depending upon whether the nucleic acid molecule is single-stranded (e.g., ssDNA or ssRNA) or double-stranded (e.g., dsDNA) and the type of nuclease activity being catalyzed by the effector protein.

The term “clustered regularly interspaced short palindromic repeats (CRISPR),” as used herein, refers to a segment of DNA found in the genomes of certain prokaryotic organisms, including some bacteria and archaea, that includes repeated short sequences of nucleotides interspersed at regular intervals between unique sequences of nucleotides derived from the DNA of a pathogen (e.g., virus) that had previously infected the organism and that functions to protect the organism against future infections by the same pathogen.

The terms “CRISPR RNA” or “crRNA,” as used herein, refer to a type of guide nucleic acid, wherein the nucleic acid is RNA comprising a first sequence that is capable of interacting with an effector protein either directly (by being bound by an effector protein) or indirectly (e.g., by hybridization with a second nucleic acid molecule that can be bound by an effector, such as a tracrRNA); and a second sequence that hybridizes to a target sequence of a target nucleic acid. In some embodiments, the first sequence is referred to as a repeat sequence and the second sequence is referred to as a spacer sequence. The first sequence and the second sequence are directly connected to each other or by a linker.

The term, “detectable signal,” as used herein, refers to a signal that can be detected using optical, fluorescent, chemiluminescent, electrochemical and other detection methods known in the art.

The term, “disrupt,” as used herein, refers to reducing or abolishing a function of a gene regulatory element by altering or modifying the nucleotide sequence of the gene regulatory element or the nucleotide sequence located in proximity (e.g., less than 200 linked nucleotides) to the gene regulatory element. In some embodiments, the gene regulatory element is a splicing-regulatory element. In some embodiments, the original function of the gene regulatory element is repressing exonic splicing. In some embodiments, there is an increased inclusion of an exon region in a mature mRNA after the disruption.

The term, “donor nucleic acid,” as used herein, refers to a nucleic acid that is (designed or intended to be) incorporated into a target nucleic acid or target sequence.

The term “dual nucleic acid system” as used herein refers to a system that uses a transactivated or transactivating RNA-crRNA duplex complexed with one or more polypeptides described herein, wherein the complex is capable of interacting with a target nucleic acid in a sequence selective manner.

The term “effector protein,” as used herein, refers to a protein, polypeptide, or peptide that is capable of interacting with a guide nucleic acid to form a complex (e.g., a RNP complex), wherein the complex interacts with a target nucleic acid. A complex between an effector protein and a guide nucleic acid can include multiple effector proteins or a single effector protein. In some embodiments, the effector protein modifies the target nucleic acid when the complex contacts the target nucleic acid. In some embodiments, the effector protein does not modify the target nucleic acid, but it is linked to a fusion partner protein that modifies the target nucleic acid when the complex contacts the target nucleic acid. A non-limiting example of an effector protein modifying a target nucleic acid is cleaving of a phosphodiester bond of the target nucleic acid. Additional examples of modifications an effector protein can make to target nucleic acids are described herein and throughout. Herein, reference to an effector protein includes reference to a nucleic acid encoding the effector protein, unless indicated otherwise.

The term, “engineered modification,” as used herein, refers to a structural change of one or more nucleic acid residues of a nucleotide sequence or one or more amino acid residue of an amino acid sequence, such as chemical modification of one or more nucleobases; or a chemical change to the phosphate backbone, a nucleotide, a nucleobase, or a nucleoside. Such modifications can be made to an effector protein amino acid sequence or guide nucleic acid nucleotide sequence, or any sequence disclosed herein (e.g., a nucleic acid encoding an effector protein or a nucleic acid that encodes a guide nucleic acid). Methods of modifying a nucleic acid or amino acid sequence are known. One of ordinary skill in the art will appreciate that the engineered modification(s) may be located at any position(s) of a nucleic acid such that the function of the nucleic acid, protein, composition, or system is not substantially decreased. Nucleic acids provided herein can be prepared according to any available technique including, but not limited to chemical synthesis, enzymatic synthesis, which is generally termed in vitro-transcription, cloning, enzymatic, or chemical cleavage, etc. In some embodiments, the nucleic acids provided herein are not uniformly modified along the entire length of the molecule. Different nucleotide modifications and/or backbone structures can exist at various positions within the nucleic acid.

An “expression cassette” comprises a DNA coding sequence operably linked to a promoter. “Operably linked” refers to a juxtaposition wherein the components so described are in a relationship permitting them to function in their intended manner. For instance, a promoter is operably linked to a coding sequence (or the coding sequence can also be said to be operably linked to the promoter) if the promoter affects its transcription or expression.

The terms “fusion protein,” or “fusion effector protein,” as used herein, refer to a protein comprising at least two heterologous polypeptides. The fusion protein may comprise one or more effector proteins and fusion partners. In some embodiments, an effector protein and fusion partner are not found connected to one another as a native protein or complex that occurs together in nature.

The term “functional domain,” as used herein, refers to a region of one or more amino acids in a protein that is required for an activity of the protein, or the full extent of that activity, as measured in an in vitro assay. Activities include, but are not limited to nucleic acid binding, nucleic acid modification, nucleic acid cleavage, protein binding. The absence of the functional domain, including mutations of the functional domain, would abolish or reduce activity.

The term, “genetic disease,” as used herein, refers to a disease, disorder, condition, or syndrome associated with or caused by one or more mutations in the DNA of an organism having the genetic disease.

The term “guide nucleic acid,” as used herein, refers to a nucleic acid comprising: a first nucleotide sequence that is capable of being non-covalently bound by an effector protein; and a second nucleotide sequence that hybridizes to a target nucleic acid. When in a complex with one or more polypeptides described herein (e.g., an RNP complex), a guide nucleic acid can impart sequence selectivity to the complex when the complex interacts with a target nucleic acid. The first sequence may be referred to herein as a repeat sequence. The second sequence may be referred to herein as a spacer sequence. The term, “guide nucleic acid,” may be used interchangeably herein with the term “guide RNA” (gRNA) however it is understood that guide nucleic acids may comprise deoxyribonucleotides (DNA), ribonucleotides (RNA), a combination thereof (e.g., RNA with a thymine base), biochemically or chemically modified nucleobases (e.g., one or more engineered modifications described herein), or combinations thereof.

The term, “handle sequence,” as used herein, refers to a sequence of nucleotides in a single guide RNA (sgRNA), that is: 1) capable of being non-covalently bound by an effector protein and 2) connects the portion of the sgRNA capable of being non-covalently bound by an effector protein to a nucleotide sequence that is hybridizable to a target nucleic acid. In general, the handle sequence comprises an intermediary RNA sequence, that is capable of being non-covalently bound by an effector protein. In some embodiments, the handle sequence further comprises a repeat sequence. In such embodiments, the intermediary RNA sequence or a combination of the intermediary RNA and the repeat sequence is capable of being non-covalently bound by an effector protein.

The term “heterologous,” as used herein, means a nucleotide or polypeptide sequence that is not found in a native nucleic acid or protein, respectively. In some embodiments, fusion proteins comprise an effector protein and a fusion partner protein, wherein the fusion partner protein is heterologous to an effector protein. These fusion proteins may be referred to as a “heterologous protein.” A protein that is heterologous to the effector protein is a protein that is not covalently linked via an amide bond to the effector protein in nature. In some embodiments, a heterologous protein is not encoded by a species that encodes the effector protein. In some embodiments, the heterologous protein exhibits an activity (e.g., enzymatic activity) when it is linked to the effector protein. In some embodiments, the heterologous protein exhibits increased or reduced activity (e.g., enzymatic activity) when it is linked to the effector protein, relative to when it is not linked to the effector protein. In some embodiments, the heterologous protein exhibits an activity (e.g., enzymatic activity) that it does not exhibit when it is linked to the effector protein. A guide nucleic acid may comprise a first sequence and a second sequence, wherein the first sequence and the second sequence are not found covalently linked via a phosphodiester bond in nature. Thus, the first sequence is considered to be heterologous with the second sequence, and the guide nucleic acid may be referred to as a heterologous guide nucleic acid.

The terms, “intermediary RNA,” “intermediary RNA sequence,” and “intermediary sequence” as used herein, in a context of a single nucleic acid system, refers to a nucleotide sequence in a handle sequence, wherein the intermediary RNA sequence is capable of, at least partially, being non-covalently bound to an effector protein to form a complex (e.g., an RNP complex). An intermediary RNA sequence is not a transactivating nucleic acid in systems, methods, and compositions described herein.

The term “linked” when used in reference to biopolymers (e.g., nucleic acids, polypeptides) refers to being covalently connected. In some embodiments, two polymers are linked by at least a covalent bond. In some embodiments, two nucleic acids are linked by at least one nucleotide. In some embodiments, two nucleic acids are linked by at least one amino acid. The terms “fused” and “linked” are used interchangeably herein.

The term “linker,” as used herein, refers to a covalent bond or molecule that links a first polypeptide to a second polypeptide (e.g., by an amide bond, or one or more amino acids) or a first nucleic acid to a second nucleic acid (e.g., by a phosphodiester bond, or one or more nucleotides).

The term “modified target nucleic acid,” as used herein, refers to a target nucleic acid, wherein the target nucleic acid has undergone a modification, for example, after contact with an effector protein. In some cases, the modification is an alteration in the sequence of the target nucleic acid. In some cases, the modified target nucleic acid comprises an insertion, deletion, or replacement of one or more nucleotides compared to the unmodified target nucleic acid.

The terms “non-naturally occurring” and “engineered,” as used herein, are used interchangeably and indicate the involvement of the hand of man. The terms, when referring to a nucleic acid, nucleotide, protein, polypeptide, peptide or amino acid, refer to a nucleic acid, nucleotide, protein, polypeptide, peptide or amino acid that is at least substantially free from at least one other feature with which it is naturally associated in nature and as found in nature, and/or contains a modification (e.g., chemical modification, nucleotide sequence, or amino acid sequence) that is not present in the naturally occurring nucleic acid, nucleotide, protein, polypeptide, peptide, or amino acid. The terms, when referring to a composition or system described herein, refer to a composition or system having at least one component that is not naturally associated with the other components of the composition or system. By way of a non-limiting example, a composition may include an effector protein and a guide nucleic acid that do not naturally occur together. Conversely, and as a non-limiting further clarifying example, an effector protein or guide nucleic acid that is “natural,” “naturally-occurring,” or “found in nature” includes an effector protein and a guide nucleic acid from a cell or organism that have not been genetically modified by the hand of man.

The term “nucleic acid expression vector,” as used herein, refers to a nucleic acid that can be used to express a nucleic acid of interest.

The term “nuclear localization signal (NLS),” as used herein, refers to an entity (e.g., peptide) that facilitates localization of a nucleic acid, protein, or small molecule to the nucleus, when present in a cell that contains a nuclear compartment.

The term “nuclease activity,” as used herein, refers to the catalytic activity that results in nucleic acid cleavage (e.g., ribonuclease activity (ribonucleic acid cleavage), or deoxyribonuclease activity (deoxyribonucleic acid cleavage), etc.).

The terms “partner protein,” “fusion partner,” or “fusion partner protein” as used herein, refer to a protein, polypeptide or peptide that is linked to an effector protein or capable of being proximal to an effector protein. In some embodiments, a fusion partner that is capable of being proximal to an effector protein is a fusion partner that is capable of binding a guide nucleic acid, wherein the effector protein is also capable of binding the guide nucleic acid. In some embodiments, a fusion partner directly interacts with (e.g., binds to/by) an effector protein. In some embodiments, a fusion partner indirectly interacts with an effector protein (e.g., through another protein or moiety).

The term “pharmaceutically acceptable excipient, carrier or diluent,” as used herein, refers to any substance formulated alongside the active ingredient of a pharmaceutical composition that allows the active ingredient to retain biological activity and is non-reactive with the subject's immune system. Such a substance can be included for the purpose of long-term stabilization, bulking up solid formulations that contain potent active ingredients in small amounts, or to confer a therapeutic enhancement on the active ingredient in the final dosage form, such as facilitating absorption, reducing viscosity, or enhancing solubility. The selection of appropriate substance can depend upon the route of administration and the dosage form, as well as the active ingredient and other factors. Compositions having such substances can be formulated by well-known conventional methods (see, e.g., Remington, The Science and Practice of Pharmacy 23^{rd} edition, A. Adejare, ed., Elsevier Publishing Co., 2020).

The terms, “promoter” and “promoter sequence,” as used herein, refer to a DNA regulatory region capable of binding RNA polymerase and initiating transcription of a downstream (3′ direction) coding or non-coding sequence. A transcription initiation site, as well as protein binding domains responsible for the binding of RNA polymerase, can also be found in a promoter region. Eukaryotic promoters will often, but not always, contain “TATA” boxes and “CAT” boxes. Various promoters, including inducible promoters, may be used to drive expression by the various vectors of the present disclosure.

The term “protospacer adjacent motif” and “PAM,” as used herein, refers to a nucleotide sequence found in a target nucleic acid that directs an effector protein to modify the target nucleic acid at a specific location. In some embodiments, a PAM sequence is required for a complex of an effector protein and a guide nucleic acid (e.g., an RNP complex) to hybridize to and edit the target nucleic acid. In some embodiments, the complex does not require a PAM to edit the target nucleic acid.

In some embodiments, the term “region” as used herein may be used to describe a portion of, or all of, a corresponding sequence, for example, a spacer region is understood to comprise a portion of or all of a spacer sequence.

The term, “regulatory element,” used herein, refers to transcriptional and translational control sequences, such as promoters, enhancers, polyadenylation signals, terminators, protein degradation signals, and the like, that provide for and/or regulate transcription of a non-coding sequence (e.g., a guide nucleic acid) or a coding sequence (e.g., effector proteins, fusion proteins, and the like) and/or regulate translation of an encoded polypeptide.

The term, “repeat sequence,” as used herein, refers to a sequence of nucleotides in a guide nucleic acid that is capable of, at least partially, interacting with an effector protein.

The terms, “ribonucleotide protein complex” and “RNP” as used herein, refer to a complex of one or more nucleic acids and one or more polypeptides described herein. While the term utilizes “ribonucleotides” it is understood that the one or more nucleic acid may comprise deoxyribonucleotides (DNA), ribonucleotides (RNA), a combination thereof (e.g., RNA with a thymine base), biochemically or chemically modified nucleobases (e.g., one or more engineered modifications described herein), or combinations thereof.

The terms, “RuvC” and “RuvC domain,” as used herein, refer to a region of an effector protein that is capable of cleaving a target nucleic acid, and in certain embodiments, of processing a pre-crRNA. In some embodiments, the RuvC domain is located near the C-terminus of the effector protein. A single RuvC domain may comprise RuvC subdomains, for example a RuvCI subdomain, a RuvCII subdomain and a RuvCIII subdomain. The term “RuvC” domain can also refer to a “RuvC-like” domain. Various RuvC-like domains are known in the art and are easily identified using online tools such as InterPro (ebi.ac.uk/interpro/). For example, a RuvC-like domain may be a domain which shares homology with a region of TnpB proteins of the IS605 and other related families of transposons

The term “sample,” as used herein, generally refers to something comprising a target nucleic acid. In some embodiments, the sample is a biological sample, such as a biological fluid or tissue sample. In some embodiments, the sample is an environmental sample. The sample may be a biological sample or environmental sample that is modified or manipulated. By way of non-limiting example, samples may be modified or manipulated with purification techniques, heat, nucleic acid amplification, salts, and buffers.

The terms, “single guide nucleic acid”, “single guide RNA” and “sgRNA,” as used herein, in the context of a single nucleic acid system, refers to a guide nucleic acid, wherein the guide nucleic acid is a single polynucleotide chain having all the required sequence for a functional complex with an effector protein (e.g., being bound by an effector protein, including in some embodiments, activating the effector protein, and hybridizing to a target nucleic acid, without the need for a second nucleic acid molecule). For example, an sgRNA can have two or more linked guide nucleic acid components (e.g., an intermediary RNA sequence, a repeat sequence, a spacer sequence and optionally a linker). In some embodiments, an sgRNA comprises a handle sequence, wherein the handle sequence comprises an intermediary sequence, a repeat sequence, and optionally a linker sequence.

The term, “single guide nucleic acid system,” as used herein, refers to a system that uses a guide nucleic acid complexed with one or more polypeptides described herein, wherein the complex is capable of interacting with a target nucleic acid in a sequence specific manner, and wherein the guide nucleic acid is capable of non-covalently interacting with the one or more polypeptides described herein, and wherein the guide nucleic acid is capable of hybridizing with a target sequence of the target nucleic acid. A single nucleic acid system lacks a duplex of a guide nucleic acid as hybridized to a second nucleic acid, wherein in such a duplex the second nucleic acid, and not the guide nucleic acid, is capable of interacting with the effector protein.

The term, “spacer sequence,” as used herein, refers to a nucleotide sequence in a guide nucleic acid that is capable of, at least partially, hybridizing to an equal length portion of a sequence (e.g., a target sequence) of a target nucleic acid. The term “spacer sequence” and “targeting sequence” are used interchangeably herein.

The term “subject,” as used herein, refers to a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a non-human primate. The mammal can be a cynomolgus monkey. The mammal can be a mouse, rat, or other rodent. The mammal can be a human. The subject may be diagnosed or suspected of being at high risk for a disease. In some embodiments, the subject is not necessarily diagnosed or suspected of being at high risk for the disease.

The term “target nucleic acid,” as used herein, refers to a nucleic acid that is selected as the nucleic acid for modification, binding, hybridization or any other activity of or interaction with a nucleic acid, protein, polypeptide, or peptide described herein. A target nucleic acid may comprise RNA, DNA, or a combination thereof. A target nucleic acid may be single-stranded (e.g., single-stranded RNA or single-stranded DNA) or double-stranded (e.g., double-stranded DNA).

The terms “target nucleic acid sequence” and “target sequence,” as used herein, when used in reference to a target nucleic acid, refers to a sequence of nucleotides found within a target nucleic acid. Such a sequence of nucleotides can, for example, hybridize to an equal length portion of a guide nucleic acid. Hybridization of the guide nucleic acid to the target sequence may bring an effector protein into contact with the target nucleic acid.

The term, “trans cleavage,” as used herein, in the context of cleavage (e.g., hydrolysis of a phosphodiester bond) of one or more target nucleic acids or non-target nucleic acids, or both, by an effector protein that is complexed with a guide nucleic acid and the target nucleic acid. Trans cleavage activity may be triggered by the hybridization of a guide nucleic acid to a target nucleic acid. The effector may cleave a target strand as well as non-target strand, wherein the target nucleic is a double stranded nucleic acid. Trans cleavage of the target nucleic acid may occur away from (e.g., not within or directly adjacent to) the portion of the target nucleic acid that is hybridized to the portion of the guide nucleic acid.

The terms, “trans-activating RNA,” “transactivating RNA,” and “tracrRNA,” refer to a transactivating or transactivated nucleic acid in a dual nucleic acid system that is capable of hybridizing, at least partially, to a crRNA to form a tracrRNA-crRNA duplex, and of interacting with an effector protein to form a complex (e.g., an RNP complex).

The terms, “transactivating,” “trans-activating,” “trans-activated,” “transactivated,” and grammatical equivalents thereof, as used herein, in the context of a dual nucleic acid system refers to an outcome of the system, wherein a polypeptide is enabled to have a binding and/or nuclease activity on a target nucleic acid, by a tracrRNA or a tracrRNA-crRNA duplex.

The term, “transcriptional activator,” as used herein, refers to a polypeptide or a fragment thereof that can activate or increase transcription of a target nucleic acid molecule.

The term “transcriptional repressor,” as used herein, refers to a polypeptide or a fragment thereof that is capable of arresting, preventing, or reducing transcription of a target nucleic acid.

The term, “transgene,” as used herein, refers to a nucleotide sequence that is inserted into a cell for expression of said nucleotide sequence in the cell. A transgene is meant to include (1) a nucleotide sequence that is not naturally found in the cell (e.g., a heterologous nucleotide sequence); (2) a nucleotide sequence that is a mutant form of a nucleotide sequence naturally found in the cell into which it has been introduced; (3) a nucleotide sequence that serves to add additional copies of the same (e.g., exogenous or homologous) or a similar nucleotide sequence naturally occurring in the cell into which it has been introduced; or (4) a silent naturally occurring or homologous nucleotide sequence whose expression is induced in the cell into which it has been introduced. A donor nucleic acid can comprise a transgene. The cell in which transgene expression occurs can be a target cell, such as a host cell.

The terms “treatment” and “treating,” as used herein, are used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying, or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

The term “viral vector,” as used herein, refers to a nucleic acid to be delivered into a host cell via a recombinantly produced virus or viral particle. The nucleic acid may be single-stranded or double stranded, linear or circular, segmented or non-segmented. The nucleic acid may comprise DNA, RNA, or a combination thereof. Non-limiting examples of viruses or viral particles that can deliver a viral vector include retroviruses (e.g., lentiviruses and γ-retroviruses), adenoviruses, arenaviruses, alphaviruses, adeno-associated viruses (AAVs), baculoviruses, vaccinia viruses, herpes simplex viruses and poxviruses. A viral vector delivered by such viruses or viral particles may be referred to by the type of virus to deliver the viral vector (e.g., an AAV viral vector is a viral vector that is to be delivered by an adeno-associated virus). A viral vector referred to by the type of virus to be delivered by the viral vector can contain viral elements (e.g., nucleotide sequences) necessary for packaging of the viral vector into the virus or viral particle, replicating the virus, or other desired viral activities. A virus containing a viral vector may be replication competent, replication deficient or replication defective.

### 2. Introduction

Disclosed herein are systems, compositions, and methods for the modification of the APOC3 gene. The APOC3 gene resides within the APOA5/APOA4/APOC3/APOA1 multigene cluster on the long arm of the human chromosome 11q23. It comprises 4 exons and 3 introns and encodes a 99 amino acid glycoprotein called apoC-III (or APOC3). This apolipoprotein is mostly expressed in hepatocytes and enterocytes, where it undergoes an intracellular cleavage, yielding the mature 79 amino acid protein. Furthermore, it undergoes a post-translational modification leading to the formation of three distinct isoforms containing zero (apoC-III0), one (apoC-III1) or two (apoC-III2) sialic acid residues, and importantly, all these isoforms exhibit the same plasma half-life and catabolic mechanisms, suggesting similar physiological implications.

At the transcriptional level, the APOC3 gene expression is tightly regulated by several proposed pathways. A series of in vivo and in vitro studies have demonstrated that its expression is downregulated by insulin, peroxisome proliferator-activated receptor α, Rev-erb, and farnesoid X receptor. Conversely, the positive responsiveness of the APOC3 promoter to glucose was reported. This factor stimulates the gene expression by the activation of the carbohydrate-responsive element binding protein, as well as the hepatocyte nuclear factor-4α. Hence, the opposite interplay between insulin and glucose on modulating APOC3 transcriptional activity may induce an enhanced apoC-III secretion under an insulin-resistant condition associated with hyperglycemia (as in type 2 diabetes). Also, the total apoC-III levels can be significantly modulated in hyperlipidemic individuals by the dietary intake of low saturated fat and high amounts of monosaturated and omega-3 polyunsaturated fatty acids. Dysregulated expression of APOC3 has been associated with dyslipidemia, hypertriglyceridemia, atherosclerosis, altered HDL functionality, and other cardiovascular disorders. Also, polymorphs of APOC3 (SstI, T-455C and C-482T) are known to associate with hypertriglyceridemia in mice, and the SstI and T-455C polymorphs significantly increased the susceptibility to CHD in humans.

Also disclosed herein are systems, compositions, and methods for the modification of the PCSK9 gene. PCSK9 is synthesized as a soluble zymogen that undergoes autocatalytic intramolecular processing in the endoplasmic reticulum. It is expressed mainly in liver, intestine, kidney, skin, and the central nervous system. After being processed in the ER, PCSK9 co-localizes with the protein sortilin on its way through the Golgi and trans-Golgi complex.

As a negative post-translational regulator of the low-density lipoprotein receptor (LDLR), PCSK9 plays a major role in cholesterol homeostasis. Upon binding of low-density lipoprotein (LDL) cholesterol to its receptor, the resulting LDLR-LDL complex is internalized. When exposed to the acidic environment within the resulting endosome LDLR adopts a hairpin conformation. This conformational change in turn induces the dissociation of the LDL-LDLR complex, allowing LDLR to be recycled back to the plasma membrane. Binding of PCSK9 binds to cell surface LDLR (through the LDLR EGF-A domain) also induces LDLR internalization. However, unlike LDL binding, PCSK9 prevents LDLR from undergoing a conformational change. This inhibition redirects LDLR to a lysosome where it is degraded. Thus, PCSK9 lowers cell surface expression of LDLR and thereby decreases metabolism of LDL-particles, which in turn may lead to hypercholesterolemia. PCSK9 also plays an important role in triglyceride-rich apoB lipoprotein production in small intestine and postprandial lipemia.

The PCSK9 gene resides on chromosome 1 at the band 1p32.3 and includes 15 exons. This gene produces two isoforms through alternative splicing. Variants of PCSK9 can reduce or increase circulating cholesterol. LDL-particles are removed from the blood when they bind to LDLR on the surface of cells, including liver cells, and are taken inside the cells. When PCSK9 binds to an LDLR, the receptor is destroyed along with the LDL particle. PCSK9 degrades LDLR by preventing the hairpin conformational change of LDLR. If PCSK9 does not bind, the receptor will return to the surface of the cell and can continue to remove LDL-particles from the bloodstream. Furthermore, PCSK9 directly promotes atherosclerosis by being involved in atherosclerotic inflammation and platelet activation.

Also disclosed herein are systems, compositions, and methods for the modification of the ANGPTL3 gene. The protein encoded by this gene is a member of the angiopoietin-like family of secreted factors. It is expressed predominantly in the liver, and has the characteristic structure of angiopoietins, consisting of a signal peptide, N-terminal coiled-coil domain, and the C-terminal fibrinogen (FBN)-like domain. The FBN-like domain in angiopoietin-like 3 protein was shown to bind alpha-5/beta-3 integrins, and this binding induced endothelial cell adhesion and migration.

In humans, ANGPTL3 is a determinant factor of HDL level and positively correlates with plasma HDL cholesterol. In genetic loss-of-function variants in only one copy of ANGPTL3, the serum LDL-C levels are reduced. In those with loss-of-function variants in both copies of ANGPTL3, low LDL-C, low HDL-C, and low triglycerides are seen (“familial combined hypolipidemia”).

In some embodiments, the present disclosure provides guide nucleic acids that are capable of binding to a target sequence in the APOC3, PCSK9, or ANGPTL genes. In some embodiments, the present disclosure provides guide nucleic acids that are capable of binding to a target sequence of the APOC3, PCSK9, or ANGPTL genes and an effector protein. In some embodiments, the effector protein is a CRISPR-associated (Cas) protein. In general, Cas proteins bind and/or modify nucleic acids in a sequence-specific manner. Cas proteins with guide nucleic acids may modify DNA at a precise target location in the genome of a wide variety of cells and organisms, allowing for precise and efficient editing of DNA sequences of interest (e.g., APOC3, PCSK9, or ANGPTL). In some embodiments, the present disclosure provides methods for treating a disease (e.g., coronary artery disease and other cardiovascular related disorders) by modifying one or more target genes (e.g., APOC3, PCSK9, or ANGPTL).

Compositions and systems disclosed herein are not naturally occurring. In general, guide nucleic acids disclosed herein are not found in nature. In some embodiments, systems and compositions herein comprise at least one non-naturally occurring component. For example, compositions and systems may comprise a guide nucleic acid, wherein the sequence of the guide nucleic acid is different or modified from that of a naturally-occurring guide nucleic acid. In some embodiments, compositions and systems comprise at least two components that do not naturally occur together. For example, compositions and systems may comprise a guide nucleic acid comprising a repeat sequence and a spacer sequence which do not naturally occur together. Also, by way of example, composition and systems may comprise a guide nucleic acid and an effector protein that do not naturally occur together. Conversely, and for clarity, an effector protein or guide nucleic acid that is “natural,” “naturally-occurring,” or “found in nature” includes effector proteins and guide nucleic acids from cells or organisms that have not been genetically modified by a human or machine.

### 3. Guide Nucleic Acids

The compositions, systems, and methods of the present disclosure may comprise a guide nucleic acid or a use thereof. Unless otherwise indicated, compositions, systems and methods comprising guide nucleic acids or uses thereof, as described herein and throughout, include DNA molecules, such as expression vectors, that encode a guide nucleic acid. Accordingly, compositions, systems, and methods of the present disclosure comprise a guide nucleic acid or a nucleotide sequence encoding the guide nucleic acid.

In general, guide nucleic acids comprise a nucleotide sequence. Such a nucleotide sequence may be described as a nucleotide sequence of either DNA or RNA, however, no matter the form the sequence is described, it is readily understood that such nucleotide sequences can be revised to be RNA or DNA, as needed, for describing a sequence within a guide nucleic acid itself or the sequence that encodes a guide nucleic acid. Similarly, disclosure of the nucleotide sequences described herein also discloses a complementary nucleotide sequence, a reverse nucleotide sequence, and the reverse complement nucleotide sequence, any one of which can be a nucleotide sequence for use in a guide nucleic acid. In some embodiments, a guide nucleic acid sequence(s) comprises one or more nucleotide alterations at one or more positions in any one of the sequences described herein. Alternative nucleotides can be any one or more of A, C, G, T or U, or a deletion, or an insertion.

A guide nucleic acid may comprise a non-naturally occurring sequence, wherein the sequence of the guide nucleic acid, or any portion thereof, may be different from the sequence of a naturally occurring guide nucleic acid. A guide nucleic acid of the present disclosure comprises one or more of the following: a) a single guide nucleic acid molecule; b) a DNA base; c) an RNA base; d) a modified base; e) a modified sugar; f) a modified backbone; and the like. Modifications are described herein and throughout the present disclosure A guide nucleic acid may be chemically synthesized or recombinantly produced by any suitable methods. Guide nucleic acids and portions thereof may be found in or identified from a CRISPR array present in the genome of a host organism or cell.

In some embodiments, the guide nucleic acid comprises a non-natural nucleobase sequence. In some embodiments, the non-natural sequence is a nucleobase sequence that is not found in nature. The non-natural sequence may comprise a portion of a naturally-occurring sequence, wherein the portion of the naturally-occurring sequence is not present in nature absent the remainder of the naturally-occurring sequence. In some embodiments, the nucleotide sequence of the guide nucleic acid is not found in nature. In some embodiments, the guide nucleic acid comprises two naturally-occurring sequences arranged in an order or proximity that is not observed in nature. In some embodiments, compositions and systems comprise a ribonucleotide complex comprising an effector protein and a guide nucleic acid that do not occur together in nature. Engineered guide nucleic acids may comprise a first sequence and a second sequence that do not occur naturally together. For example, a guide nucleic acid may comprise a sequence of a naturally-occurring repeat region and a spacer region that is complementary to a naturally-occurring eukaryotic sequence. The guide nucleic acid may comprise a sequence of a repeat region that occurs naturally in an organism and a spacer region that does not occur naturally in that organism. A guide nucleic acid may comprise a first sequence that occurs in a first organism and a second sequence that occurs in a second organism, wherein the first organism and the second organism are different. The guide nucleic acid may comprise a third sequence disposed at a 3′ or 5′ end of the guide nucleic acid, or between the first and second sequences of the guide nucleic acid. In some embodiments, a guide nucleic acid is a crRNA, wherein the crRNA comprises a repeat sequence and a spacer sequence that is complementary to a eukaryotic target sequence. In some embodiments, a guide nucleic acid may comprise a repeat sequence, an intermediary sequence, and a spacer sequence coupled by one or more linker sequences. In some embodiments, the guide nucleic acid comprises two heterologous sequences arranged in an order or proximity that is not observed in nature. Therefore, guide nucleic acid compositions described herein are not naturally occurring.

In general, a guide nucleic acid comprises a first nucleotide sequence that is capable of being non-covalently bound by an effector protein and a second nucleotide sequence that hybridizes to a target nucleic acid. In some embodiments, the first nucleotide sequence is located 5′ to second nucleotide sequence. In some embodiments, the second nucleotide sequence is located 5′ to first nucleotide sequence. In some embodiments, the first nucleotide sequence comprises a repeat sequence. In some embodiments, the first nucleotide sequence comprises an intermediary sequence. In some embodiments, an effector protein binds to at least a portion of the first nucleotide sequence. In some embodiments, the second nucleotide sequence comprises a spacer sequence, wherein the spacer sequence can interact in a sequence-specific manner with (e.g., has complementarity with, or can hybridize to a target sequence in) a target nucleic acid (e.g., the APOC3, PCSK9, or ANGPTL3 genes). Although the term may imply that a gRNA consists of RNA, in some embodiments, a gRNA may comprise one or more deoxyribonucleotides and/or a deoxyribonucleotide nucleobase (e.g., thymine). However, the majority of the nucleotides in a guide nucleic acid (at least 50%) are ribonucleotides.

Modifications can further include changing of nucleic acids described herein (e.g., engineered guide nucleic acids) to provide the nucleic acid with a new or enhanced feature, such as improved stability. Such modifications of a nucleic acid include a nucleobase base modification, a backbone modification, a sugar modification, or combinations thereof. In some embodiments, the modifications can be of one or more nucleotides, nucleosides, or nucleobases in a nucleic acid. In some embodiments, uridines can be exchanged for pseudouridines (e.g., 1N-Methyl-Pseudouridine). In some embodiments, all uridines can be exchanged for 1N-Methyl-Pseudouridine. In this application, U can represent uracil or 1N-Methyl-Pseudouridine.

The guide nucleic acid may also form complexes as described through herein. For example, a guide nucleic acid may hybridize to another nucleic acid, such as target nucleic acid, or a portion thereof. In another example, a guide nucleic acid may complex with an effector protein. In such embodiments, a guide nucleic acid-effector protein complex may be described herein as an RNP. In some embodiments, when in a complex, at least a portion of the complex may bind, recognize, and/or hybridize to a target nucleic acid (e.g., a target sequence in the APOC3, PCSK9, or ANGPTL3 genes). For example, when a guide nucleic acid and an effector protein are complexed to form an RNP, at least a portion of the guide nucleic acid hybridizes to a target sequence in a target nucleic acid (e.g., the APOC3, PCSK9, or ANGPTL3 genes). Those skilled in the art in reading the below specific examples of guide nucleic acids as used in RNPs described herein, will understand that in some embodiments, a RNP may hybridize to one or more target sequences in a target nucleic acid, thereby allowing the RNP to modify and/or recognize a target nucleic acid or sequence contained therein (e.g., PAM) or to modify and/or recognize non-target sequences depending on the guide nucleic acid, and in some embodiments, the effector protein, used.

In some embodiments, a guide nucleic acid may comprise or form intramolecular secondary structure (e.g., hairpins, stem-loops, etc.). In some embodiments, a guide nucleic acid comprises a stem-loop structure comprising a stem region and a loop region. In some embodiments, the stem region is 4 to 8 linked nucleotides in length. In some embodiments, the stem region is 5 to 6 linked nucleotides in length. In some embodiments, the stem region is 4 to 5 linked nucleotides in length. In some embodiments, the guide nucleic acid comprises a pseudoknot (e.g., a secondary structure comprising a stem, at least partially, hybridized to a second stem or half-stem secondary structure). An effector protein may recognize a guide nucleic acid comprising multiple stem regions. In some embodiments, the nucleotide sequences of the multiple stem regions are identical to one another. In some embodiments, the nucleotide sequences of at least one of the multiple stem regions is not identical to those of the others. In some embodiments, the guide nucleic acid comprises at least 2, at least 3, at least 4, or at least 5 stem regions.

In some embodiments, the compositions, systems, and methods of the present disclosure comprise two or more guide nucleic acids (e.g., 2, 3, 4, 5, 6, 7, 9, 10 or more guide nucleic acids), and/or uses thereof. Multiple guide nucleic acids may target an effector protein to different loci in the target nucleic acid by hybridizing to different target sequences. In some embodiments, a first guide nucleic acid may hybridize within a location of the target nucleic acid that is different from where a second guide nucleic acid may hybridize the target nucleic acid. In some embodiments, the first loci and the second loci of the target nucleic acid may be located at least 1, at least 10, at least 20, at least 30, at least 40, at least 50, at least 60, at least 70, at least 80, at least 90 or at least 100 nucleotides apart. In some embodiments, the first loci and the second loci of the target nucleic acid may be located between 100 and 200, 200 and 300, 300 and 400, 400 and 500, 500 and 600, 600 and 700, 700 and 800, 800 and 900 or 900 and 1000 nucleotides apart.

In some embodiments, the first loci and/or the second loci of the target nucleic acid are located in an intron of a gene (e.g., an intron of the APOC3, PCSK9, or ANGPTL3 genes). In some embodiments, the first loci and/or the second loci of the target nucleic acid are located in an exon of a gene (e.g., an exon of the APOC3, PCSK9, or ANGPTL3 genes). In some embodiments, the first portion and/or the second portion of the target nucleic acid are located on either side of an exon and cutting at both sites results in deletion of the exon. In some embodiments, composition, systems, and methods comprise a donor nucleic acid that may be inserted in replacement of a deleted or cleaved sequence of the target nucleic acid. In some embodiments, compositions, systems, and methods comprising multiple guide nucleic acids or uses thereof comprise multiple effector proteins, wherein the effector proteins may be identical, non-identical, or combinations thereof.

In some embodiments, the guide nucleic acid comprises about: 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 linked nucleotides. In general, the guide nucleic acid comprises at least: 10, 15, 20, 25, 30, 35, 40, 45, 50, 55, 60 linked nucleotides. In some embodiments, the guide nucleic acid comprises about 10 to about 60, about 20 to about 50, or about 30 to about 40 linked nucleotides. In some embodiments, the guide nucleic acid comprises at least 25 linked nucleotides.

A guide nucleic acid may comprise 10 to 50 linked nucleotides. In some embodiments, the guide nucleic acid comprises or consists essentially of about 12 to about 80 linked nucleotides, about 12 to about 50, about 12 to about 45, about 12 to about 40, about 12 to about 35, about 12 to about 30, about 12 to about 25, from about 12 to about 20, about 12 to about 19, about 19 to about 20, about 19 to about 25, about 19 to about 30, about 19 to about 35, about 19 to about 40, about 19 to about 45, about 19 to about 50, about 19 to about 60, about 20 to about 25, about 20 to about 30, about 20 to about 35, about 20 to about 40, about 20 to about 45, about 20 to about 50, or about 20 to about 60 linked nucleotides. In some embodiments, the guide nucleic acid comprises about 10 to about 60, about 20 to about 50, or about 30 to about 40 linked nucleotides.

In some embodiments, a length of a guide nucleic acid is about 30 to about 120 linked nucleotides. In some embodiments, the length of a guide nucleic acid is about 40 to about 100, about 40 to about 90, about 40 to about 80, about 40 to about 70, about 40 to about 60, about 40 to about 50, about 50 to about 90, about 50 to about 80, about 50 to about 70, or about 50 to about 60 linked nucleotides. In some embodiments, the length of a guide nucleic acid is about 40, about 45, about 50, about 55, about 60, about 65, about 70 or about 75 linked nucleotides. In some embodiments, the length of a guide nucleic acid is greater than about 15, about 20, about 25, about 30, about 35, about 40, about 45, about 50, about 55, about 60, about 65, about 70 or about 75 linked nucleotides. In some embodiments, the length of a guide nucleic acid is not greater than about 40, about 45, about 50, about 55, about 60, about 65, about 70, about 75, about 80, about 85, about 90, about 95, about 100, about 105, about 110, about 115, about 120, or about 125 linked nucleotides.

In some embodiments, guide nucleic acids comprise elements that contribute functionality (e.g., stability, heat resistance, etc.) to the guide nucleic acid. Such elements may be one or more nucleotide alterations, nucleotide sequences, intermolecular secondary structures, or intramolecular secondary structures (e.g., one or more hair pin regions, one or more bulges, etc.).

In some embodiments, guide nucleic acids comprise one or more linkers connecting different nucleotide sequences as described herein. A linker may comprise 1, 2, 3, 4, 5, 6, 7, 8, 9, 10, or more nucleotides. A linker may be any suitable linker, examples of which are described herein.

Guide nucleic acids may comprise deoxyribonucleotides, ribonucleotides or a combination thereof. In some embodiments, a guide nucleic acid comprises a ribonucleotide with a thymine nucleobase. Guide nucleic acids may comprise a chemically modified nucleobase or phosphate backbone. Guide nucleic acids may be referred to herein as a guide RNA (gRNA). However, a guide RNA is not limited to ribonucleotides, but may comprise deoxyribonucleotides and other chemically modified nucleotides. A guide nucleic acid may comprise a non-naturally occurring guide nucleic acid, including a guide nucleic acid that is designed to contain a chemical or biochemical modification.

In some embodiments, effector proteins are targeted by a guide nucleic acid (e.g., a guide RNA) to a specific location in the target nucleic acid where they exert locus-specific nucleotide modification or gene regulation. Non-limiting examples of gene regulation include blocking RNA polymerase binding to a promoter (which selectively inhibits transcription activator function), and/or modifying local chromatin (e.g., modifying the target nucleic acid or modifying a protein associated with the target nucleic acid). The guide RNA may bind to a target nucleic acid (e.g., a single strand of a target nucleic acid) or a portion thereof, an amplicon thereof, or a portion thereof. By way of non-limiting example, a guide nucleic acid may bind to a portion of a gene associated with a genetic disorder, or an amplicon thereof, as described herein.

In some embodiments, the compositions, systems, and methods of the present disclosure may comprise an additional guide nucleic acid or a use thereof. An additional guide nucleic acid can target an effector protein to a different location in the target nucleic acid (e.g., APOC3, PCSK9, and ANGPTL3 genes) by binding to a different portion of the target nucleic acid from the first guide nucleic acid. A system in which two different guide nucleic acids are used to target two different locations in the target nucleic acid may be referred to as a dual guided system. In certain embodiments, upon removal of a sequence between two guide nucleic acids, the wild-type reading frame may be restored, e.g., by a polymerase, resulting in at least a partially functional protein.

### Single Guide Nucleic Acid Systems

In some embodiments, compositions, systems, and methods described herein comprise a single guide nucleic acid. In the single guide nucleic acid system, the effector protein is not transactivated by a guide nucleic acid. By way of non-limiting example, a single guide nucleic acid system does not require a tracrRNA. In other words, activity of the effector protein does not require binding to a second or intermediary guide nucleic acid molecule. Exemplary guide nucleic acids for a single guide nucleic acid system are crRNAs and sgRNAs.

crRNA

In some embodiments, the single guide nucleic acid comprises a crRNA. In general, a crRNA comprises a first region (FR) and a second region (SR), wherein the FR of the crRNA comprises a repeat sequence, and the SR of the crRNA comprises a spacer sequence. In some embodiments, the spacer sequence follows the repeat sequence in a 5′ to 3′ direction. In some embodiments, the spacer sequence precedes the repeat sequence in a 5′ to 3′ direction. In some embodiments, the repeat sequence and the spacer sequences are directly connected to each other (e.g., covalent bond (phosphodiester bond)). In some embodiments, the repeat sequence and the spacer sequence are connected by a linker.

In some embodiments, a crRNA is useful as a single guide nucleic acid system for compositions, methods, and systems described herein or as part of a single guide nucleic acid system for compositions, methods, and systems described herein. In such embodiments, a single guide nucleic acid system comprises a guide nucleic acid comprising a crRNA wherein, a repeat sequence of a crRNA is capable of causing a crRNA to interact with an effector protein. In some embodiments, a single guide nucleic acid system comprises a guide nucleic acid comprising a crRNA linked to another nucleotide sequence that is capable of being non-covalently bound by an effector protein. In some embodiments, a crRNA is sufficient to form complex with an effector protein (e.g., to form an RNP) through the repeat sequence and direct the effector protein to a target nucleic acid sequence through the spacer sequence.

In some embodiments, compositions and systems described herein comprise an effector protein or a nucleic acid encoding the effector protein, wherein the effector protein comprises an amino acid sequence that is at least 75%, at least 80%, at least 85%, at least 90%, at least 95%, at least 97%, at least 98%, at least 99% or 100% identical to any one of SEQ ID NOs: 32-33, 34-35, 45-46, 54-66, 203-204, 794, and 2090-2091; and a guide nucleic acid that consists essentially of a crRNA. In some embodiments, the crRNA comprises a sequence that is at least 70%, at least 75%, at least 80%, at least 85%, at least 90%, at least 95%, or 100% identical to a sequence selected from SEQ ID NOs: 1-31, 38-43, 67-202, 207-208, 491-493, 799-820, 830-999 and 1400-1569. In some embodiments, the crRNA consists of a sequence that is at least 70%, at least 75%, at least 80%, at least 85%, at least 90%, at least 95%, or 100% identical to a sequence selected from SEQ ID NOs: 1-31, 38-43, 67-202, 207-208, 491-493, 799-820, 830-999 and 1400-1569.

A crRNA may include deoxyribonucleosides, ribonucleosides, chemically modified nucleosides, or any combination thereof. In some embodiments, a crRNA comprises about: 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 linked nucleotides. In some embodiments, a crRNA comprises at least: 10, 15, 20, 25, 30, 35, 40, 45, 50, 55, 60 linked nucleotides. In some embodiments, the length of the crRNA is about 20 to about 120 linked nucleotides. In some embodiments, the length of a crRNA is about 20 to about 100, about 30 to about 100, about 40 to about 100, about 40 to about 90, about 40 to about 80, about 40 to about 70, about 40 to about 60, about 40 to about 50, about 50 to about 90, about 50 to about 80, about 50 to about 70, or about 50 to about 60 linked nucleotides. In some embodiments, the length of a crRNA is about 20, about 25, about 30, about 35, about 40, about 45, about 50, about 55, about 60, about 65, about 70 or about 75 linked nucleotides.

sgRNA

In some embodiments, a guide nucleic acid comprises a single guide RNA (sgRNA). In some embodiments, an sgRNA can have two or more linked guide nucleic acid components (e.g., an intermediary RNA sequence, a repeat sequence, a spacer sequence, and optionally a linker). In some embodiments, an sgRNA comprises a handle sequence, wherein the handle sequence comprises an intermediary sequence, a repeat sequence, and optionally a linker sequence. In some embodiments, the guide nucleic acid is an sgRNA. The combination of a spacer sequence (e.g., a nucleotide sequence that hybridizes to a target sequence in a target nucleic acid) with a handle sequence may be referred to herein as a single guide RNA (sgRNA), wherein the spacer sequence and the handle sequence are covalently linked. In some embodiments, the spacer sequence and handle sequence are linked by a phosphodiester bond. In some embodiments, the spacer sequence and handle sequence are linked by one or more linked nucleotides. In some embodiments, a guide nucleic acid may comprise a spacer sequence, a repeat sequence, or handle sequence, or a combination thereof. In some embodiments, the handle sequence may comprise a portion of, or all of, a repeat sequence. In general, an sgRNA comprises a first region (FR) and a second region (SR), wherein the FR comprises a handle sequence and the SR comprises a spacer sequence.

In some embodiments, the compositions comprising a guide RNA and an effector protein without a tracrRNA (e.g., a single nucleic acid system), wherein the guide RNA is an sgRNA. An sgRNA may include deoxyribonucleosides, ribonucleosides, chemically modified nucleosides, or any combination thereof. An sgRNA may also include a nucleotide sequence that forms a secondary structure (e.g., one or more hairpin loops) that facilitates the binding of an effector protein to the sgRNA and/or modification activity of an effector protein on a target nucleic acid (e.g., a hairpin region). Such a sequence can be contained within a handle sequence as described herein.

In some embodiments, an sgRNA comprises one or more of one or more of a handle sequence, an intermediary sequence, a crRNA, a repeat sequence, a spacer sequence, a linker, or combinations thereof. For example, an sgRNA comprises a handle sequence and a spacer sequence; an intermediary sequence and a crRNA; an intermediary sequence, a repeat sequence, and a spacer sequence; and the like.

In some embodiments, sgRNA comprises an intermediary sequence and a crRNA. In some embodiments, an intermediary sequence is 5′ to a crRNA in an sgRNA. In some embodiments, an sgRNA comprises a linked intermediary sequence and crRNA. In some embodiments, an intermediary sequence and a crRNA are linked in an sgRNA directly (e.g., covalently linked intermediary sequence and crRNA. In some embodiments, an intermediary sequence and a crRNA are linked in an sgRNA directly (e.g., covalently linked, such as through a phosphodiester bond) In some embodiments, an intermediary sequence and a crRNA are linked in an sgRNA by any suitable linker, examples of which are provided herein.

In some embodiments, an sgRNA comprises a handle sequence and a spacer sequence. In some embodiments, a handle sequence is 5′ to a spacer sequence in an sgRNA. In some embodiments, an sgRNA comprises a linked handle sequence and spacer sequence. In some embodiments, a handle sequence and a spacer sequence are linked in an sgRNA directly (e.g., covalently linked, such as through a phosphodiester bond) In some embodiments, a handle sequence and a spacer sequence are linked in an sgRNA by any suitable linker, examples of which are provided herein.

In some embodiments, an sgRNA comprises an intermediary sequence, a repeat sequence, and a spacer sequence. In some embodiments, an intermediary sequence is 5′ to a repeat sequence in an sgRNA. In some embodiments, an sgRNA comprises a linked intermediary sequence and repeat sequence. In some embodiments, an intermediary sequence and a repeat sequence are linked in an sgRNA directly (e.g., covalently linked, such as through a phosphodiester bond) In some embodiments, an intermediary sequence and a repeat sequence are linked in an sgRNA by any suitable linker, examples of which are provided herein. In some embodiments, a repeat sequence is 5′ to a spacer sequence in an sgRNA. In some embodiments, an sgRNA comprises a linked repeat sequence and spacer sequence. In some embodiments, a repeat sequence and a spacer sequence are linked in an sgRNA directly (e.g., covalently linked, such as through a phosphodiester bond) In some embodiments, a repeat sequence and a spacer sequence are linked in an sgRNA by any suitable linker, examples of which are provided herein.

An exemplary handle sequence in an sgRNA may comprise, from 5′ to 3′, a 5′ region, a hairpin region, and a 3′ region. In some embodiments, the 5′ region may hybridize to the 3′ region. In some embodiments, the 5′ region does not hybridize to the 3′ region. In some embodiments, the 3′ region is covalently linked to a spacer sequence (e.g., through a phosphodiester bond). In some embodiments, the 5′ region is covalently linked to a spacer sequence (e.g., through a phosphodiester bond).

In some embodiments, compositions and systems described herein comprise an effector protein or a nucleic acid encoding the effector protein, wherein the effector protein comprises an amino acid sequence that is at least 75%, at least 80%, at least 85%, at least 90%, at least 95%, at least 97%, at least 98%, at least 99% or 100% identical to any one of SEQ ID NOs: 773-776 and 778-793; and a guide nucleic acid that comprises an sgRNA. In some embodiments, the sgRNA comprises a sequence that is at least 70%, at least 75%, at least 80%, at least 85%, at least 90%, at least 95%, or 100% identical to a sequence selected from SEQ ID NOs: 44, 209-490, 494-772, 822-829, 1000-1399, and 1570-2086. In some embodiments, the sgRNA consists of a sequence that is at least 70%, at least 75%, at least 80%, at least 85%, at least 90%, at least 95%, or 100% identical to a sequence selected from SEQ ID NOs: 44, 209-490, 494-772, 822-829, 1000-1399, and 1570-2086.

### Dual Nucleic Acid Systems

In some embodiments, compositions, systems and methods described herein comprise a dual nucleic acid system comprising a crRNA or a nucleotide sequence encoding the crRNA, a tracrRNA, or a nucleotide sequence encoding the tracrRNA, and one or more effector protein or a nucleotide sequence encoding the one or more effector protein, wherein the crRNA and the tracrRNA are separate, unlinked molecules, wherein a repeat hybridization region of the tracrRNA is capable of hybridizing with an equal length portion of the crRNA to form a tracrRNA-crRNA duplex, wherein the equal length portion of the crRNA does not include a spacer sequence of the crRNA, and wherein the spacer sequence is capable of hybridizing to a target sequence of the target nucleic acid. In the dual nucleic acid system having a complex of the guide nucleic acid, tracrRNA, and the effector protein, the effector protein is transactivated by the tracrRNA. In other words, in a dual nucleic acid system, activity of the effector protein requires binding to a tracrRNA molecule.

In some embodiments, a repeat hybridization sequence is at the 3′ end of a tracrRNA sequence. In some embodiments, a repeat hybridization sequence may have a length of about 1, about 2, about 3, about 4, about 5, about 6, about 7, about 8, about 9, about 10, about 12, about 14, about 16, about 18, or about 20 linked nucleotides. In some embodiments, the length of the repeat hybridization sequence is 1 to 20 linked nucleotides.

A tracrRNA and/or tracrRNA-crRNA duplex may form a secondary structure that facilitates the binding of an effector protein to a tracrRNA or a tracrRNA-crRNA. In some embodiments, the secondary structure modifies activity of the effector protein on a target nucleic acid. In some embodiments, the secondary structure comprises a stem-loop structure comprising a stem region and a loop region. In some embodiments, the stem region is 4 to 8 linked nucleotides in length. In some embodiments, the stem region is 5 to 6 linked nucleotides in length. In some embodiments, the stem region is 4 to 5 linked nucleotides in length. In some embodiments, the secondary structure comprises a pseudoknot (e.g., a secondary structure comprising a stem at least partially hybridized to a second stem or half-stem secondary structure). An effector protein may recognize a secondary structure comprising multiple stem regions. In some embodiments, nucleotide sequences of the multiple stem regions are identical to one another. In some embodiments, the nucleotide sequences of at least one of the multiple stem regions is not identical to those of the others. In some embodiments, the secondary structure comprises at least two, at least three, at least four, or at least five stem regions. In some embodiments, the secondary structure comprises one or more loops. In some embodiments, the secondary structure comprises at least one, at least two, at least three, at least four, or at least five loops.

### Spacer Sequences

Guide nucleic acids described herein may comprise one or more spacer sequences (spacer sequences are also referred to throughout this specification as “targeting sequences” and the two terms are interchangeable). In some embodiments, a spacer sequence is capable of hybridizing to a target sequence of a target nucleic acid. In some embodiments, a spacer sequence comprises a nucleotide sequence that is, at least partially, hybridizable to an equal length of a sequence (e.g., a target sequence) of a target nucleic acid. Exemplary hybridization conditions are described herein. In some embodiments, the spacer sequence may function to direct an RNP complex comprising the guide nucleic acid to the target nucleic acid for detection and/or modification. The spacer sequence may function to direct a RNP to the target nucleic acid for detection and/or modification. A spacer sequence may be complementary to a target sequence that is adjacent to a PAM that is recognizable by an effector protein described herein.

The spacer sequence of a guide nucleic acid is complementary to a target sequence of a target nucleic acid. The spacer sequence of a guide nucleic acid may be at least 70%, at least 75%, at least 80%, at least 85%, at least 90%, at least 95%, or 100% complementary to a target sequence of a target nucleic acid. In general, the spacer sequence is capable of hybridizing to a target sequence of a target nucleic acid. It is understood that the spacer sequence need not be 100% complementary to that of a target sequence of a target nucleic acid to hybridize or hybridize specifically to the target sequence.

In some embodiments, the spacer region is 5-50 linked nucleotides in length. In some embodiments, the spacer region is 15-28 linked nucleotides in length. In some embodiments, the spacer region is 15-26, 15-24, 15-22, 15-20, 15-18, 16-28, 16-26, 16-24, 16-22, 16-20, 16-18, 17-26, 17-24, 17-22, 17-20, 17-18, 18-26, 18-24, or 18-22 linked nucleotides in length. In some embodiments, the spacer region is 18-24 linked nucleotides in length. In some embodiments, the spacer region is at least 15 linked nucleotides in length. In some embodiments, the spacer region is at least 16, 18, 20, or 22 linked nucleotides in length. In some embodiments, the spacer region comprises at least 10, 11, 12, 13, 14, 15, 16, 17, 18, 19, 20, 21, 22, 23, 24, 25, 26, 27, 28, 29, or 30 nucleotides. In some embodiments, the spacer region is at least 17 linked nucleotides in length. In some embodiments, the spacer region is at least 18 linked nucleotides in length. In some embodiments, the spacer region is at least 20 linked nucleotides in length. In some embodiments, the spacer region is at least 80%, at least 85%, at least 90%, at least 95% or 100% complementary to a target sequence of the target nucleic acid. In some embodiments, the spacer region is 100% complementary to the target sequence of the target nucleic acid. In some embodiments, the spacer region comprises at least 15 contiguous nucleobases that are complementary to the target nucleic acid.

In some embodiments, a spacer sequence is adjacent to a repeat sequence. In some embodiments, a spacer sequence follows a repeat sequence in a 5′ to 3′ direction. In some embodiments, a spacer sequence precedes a repeat sequence in a 5′ to 3′ direction. In some embodiments, the spacer sequence(s) and the repeat sequence(s) of the guide nucleic acid are present within the same molecule. In some embodiments, the spacer(s) and repeat sequence(s) are linked directly to one another. In some embodiments, a linker is present between the spacer(s) and repeat sequences. Linkers may be any suitable linker. In some embodiments, the spacer sequence(s) and the repeat sequence(s) of the guide nucleic acid are present in separate molecules, which are joined to one another by base pairing interactions.

In some embodiments, a spacer sequence comprises a nucleotide sequence that is at least 65%, at least 70%, at least 75%, at least 80%, at least 85%, at least 90%, at least 95% or 100% complementary to a target sequence of a target nucleic acid (e.g., the APOC3, PCSK9, or ANGPTL3 genes). A spacer sequence is capable of hybridizing to an equal length portion of a target nucleic acid (e.g., a target sequence). In some embodiments, a spacer sequence comprises a sequence that is at least 65%, at least 70%, at least 75%, at least 80%, at least 85%, at least 90%, at least 95% or 100% complementary to a target sequence of an APOC3 target nucleic acid. In some embodiments, a spacer sequence comprises a sequence that is at least 65%, at least 70%, at least 75%, at least 80%, at least 85%, at least 90%, at least 95% or 100% complementary to a target sequence of a PCKS9 target nucleic acid. In some embodiments, a spacer sequence comprises a sequence that is at least 65%, at least 70%, at least 75%, at least 80%, at least 85%, at least 90%, at least 95% or 100% complementary to a target sequence of a ANGPTL3 target nucleic acid. In some embodiments, the spacer sequence comprises at least 10, at least 11, at least 12, at least 13, at least 14, at least 15, at least 16, at least 17, at least 18, at least 19, or at least 20 contiguous nucleotides that are capable of hybridizing to the target sequence. In some embodiments, the spacer sequence comprises at least 10, at least 11, at least 12, at least 13, at least 14, at least 15, at least 16, at least 17, at least 18, at least 19, or at least 20 contiguous nucleotides that are complementary to the target sequence.

### APOC3 Spacer Sequences

TABLE 1 and TABLE 2 provides illustrative spacer sequences targeting the APOC3 gene for use with the compositions, systems, and methods of the disclosure. In particular, TABLE 1 provides spacer sequences suitable for use in combination with an effector protein of SEQ ID NO: 32 or variants thereof (e.g., variants provided in TABLES 18 and 19). In particular, TABLE 2 provides spacer sequences suitable for use in combination with an effector protein of SEQ ID NO: 773 or variants thereof (e.g., variants provided in TABLES 16 and 17). In some embodiments, the spacer sequence comprises at least 70%, at least 80%, at least 90%, at least 91%, at least 92%, at least 93%, at least 94%, at least 95%, at least 96%, at least 97%, or at least 99%, or 100% sequence identity to a sequence as set forth in TABLE 1 or TABLE 2. In some embodiments, spacer sequences comprise at least 10, at least 11, at least 12, at least 13, at least 14, at least 15, at least 16, at least 17, at least 18, at least 19, or at least 20 contiguous nucleotides of a sequence selected from any one of SEQ ID NOs: 1-15, 67-72, 207, 209-299, 804-805, 823-825, 830-1399, 2018-2026, and 2084-2086.

Guide nucleic acids disclosed herein may target various regions of the APOC3 gene. In some embodiments, spacer sequences are complementary to a target sequence in exon 1 of APOC3. In some embodiments, spacer sequences hybridize to a target sequence in exon 1 of APOC3. By way of non-limiting example, spacer sequences that are complementary to exon 1 of APOC3 include SEQ ID NOs: 209-211. In some embodiments, spacer sequences are complementary to a target sequence in exon 2 of APOC3. In some embodiments, spacer sequences hybridize to a target sequence in exon 2 of APOC3. By way of non-limiting example, spacer sequences that are complementary to exon 2 of APOC3 includes SEQ ID NO: 212. In some embodiments, spacer sequences are complementary to a target sequence in exon 3 of APOC3. In some embodiments, spacer sequences hybridize to a target sequence in exon 3 of APOC3. By way of non-limiting example, spacer sequences that are complementary to exon 3 of APOC3 include SEQ ID NOs: 213-217. In some embodiments, spacer sequences are complementary to a target sequence in exon 4 of APOC3. In some embodiments, spacer sequences hybridize to a target sequence in exon 4 of APOC3. By way of non-limiting example, spacer sequences that are complementary to exon 4 of APOC3 include SEQ ID NOs: 1-15 and 218-269. In some embodiments, spacer sequences are complementary to a splice donor site of exon 1 of APOC3. In some embodiments, spacer sequences hybridize to a splice donor site of exon 1 of APOC3. By way of non-limiting example, spacer sequences that are complementary to a splice donor site of exon 1 of APOC3 include SEQ ID NO: 67, 68, and 270-280. In some embodiments, spacer sequences are complementary to a splice donor site of exon 2 of APOC3. In some embodiments, spacer sequences hybridize to a splice donor site of exon 2 of APOC3. By way of non-limiting example, spacer sequences that are complementary to a splice donor site of exon 2 of APOC3 include SEQ ID NOs: 69, 207, and 296. In some embodiments, spacer sequences are complementary to a splice donor site of exon 3 of APOC3. In some embodiments, spacer sequences hybridize to a splice donor site of exon 3 of APOC3. By way of non-limiting example, spacer sequences that are complementary to a splice donor site of exon 3 of APOC3 include SEQ ID NO: 70, 71, and 281-295. In some embodiments, spacer sequences are complementary to a splice donor site of exon 4 of APOC3. In some embodiments, spacer sequences hybridize to a splice donor site of exon 4 of APOC3. By way of non-limiting example, spacer sequences that are complementary to a splice donor site of exon 4 of APOC3 include SEQ ID NOs: 72 and 297.

TABLE 1
Exemplary Spacer Sequences Targeting APOC3 for
CasPhi.12 Effector Proteins

| | Spacer sequence | SEQ |
|---|---|---|
| Spacer ID | (5′ to 3′), shown as RNA | ID NO: |
| 114178 | UCCUUAACGGUGCUCCA | 1 |
| 114179 | ACGGUGCUCCAGUAGUC | 2 |
| n/a | AAGCAACCUACAGGGGC | 3 |
| n/a | UCCAGCUUUAUUGGGAG | 4 |
| n/a | GGGUAUUGAGGUCUCAG | 5 |
| n/a | AGCAACCUACAGGGGCA | 6 |
| 114188 | AGGGAACUGAAGCCAUC | 7 |
| n/a | UAAGCAACCUACAGGGG | 8 |
| n/a | UUGTCCAGCUUUAUUGG | 9 |
| 114193 | CAGGGAACUGAAGCCAU | 10 |
| 114195 | CCUGAAAGACUACUGGA | 11 |
| n/a | AAAGGGACAGUAUUCUC | 12 |
| n/a | CUUAAAAGGGACAGUAU | 13 |
| 114201 | AGUUCCCUGAAAGACUA | 14 |
| n/a | AUCCCUAGAGGCAGCUG | 15 |
| 114212 | CCCUCCCCAGAGGGCAU | 67 |
| 114230 | CCCCUCCCCAGAGGGCA | 68 |
| 114260 | CUUGCAGGAACAGAGGC | 69 |
| 127527 | CCUCAGGAGCUUCAGAG | 70 |
| 127528 | CUCAGGAGCUUCAGAGG | 71 |
| 127529 | UCAUGCCCUGCUCUGUU | 72 |
| n/a | GUGGGACUGGGCUGGGG | 207 |
| PL34716 | CUUGCAGGAACAGAGGUGCC | 804 |
| PL34717 | CCUCAGGAGCUUCAGAGGCC | 805 |
| 132842 | CCCAACUCUCCCGCCCG | 830 |
| 132843 | AGGCUUAGGGCUGGAGG | 831 |
| 132844 | CCCUCUCACCAGCCUCU | 832 |
| 132845 | AGGGCUUGGGGCUGGUG | 833 |
| 132846 | CUCCAAACACCCCCCAG | 834 |
| 132847 | GGGCUGGAGGAAGCCUU | 835 |
| 132848 | CCAACUCUCCCGCCCGC | 836 |
| 132849 | GCUGGACUGGACGGAGA | 837 |
| 132850 | UCUGCUCCAUCCCACCC | 838 |
| 132851 | CCCAGCGCCCUGGGUCC | 839 |
| 132852 | UGUGCCUUUACUCCAAA | 840 |
| 132853 | CUGCAUCUGGACACCCU | 841 |
| 132854 | CUAGAGCUAAGGAAGCC | 842 |
| 132855 | GCCCAGCGCCCUGGGUC | 843 |
| 132856 | CAGUGUGAAAGGCUGAG | 844 |
| 132857 | UUCAGGCUUAGGGCUGG | 845 |
| 132858 | GGGCCUCGAUCCCUCGC | 846 |
| 132859 | ACUCCAAACACCCCCCA | 847 |
| 132860 | AGUCUGGUGGGUUUUCU | 848 |
| 132861 | CCCAAAGCUACACAGGG | 849 |
| 132862 | UGCUCCAUCCCACCCAC | 850 |
| 132863 | AUGUUCAGUCUGGUGGG | 851 |
| 132864 | CUGCUCCAUCCCACCCA | 852 |
| 132865 | AUCCCUAGAGGCAGCUG | 853 |
| 132866 | GACAGCCCAGUCCUACC | 854 |
| 132867 | GGGCUGGUGGAGGGAGG | 855 |
| 132868 | CUGAGCUCAUCUGGGCU | 856 |
| 132869 | GGCCUCGAUCCCUCGCC | 857 |
| 132870 | UCAAGUCUGAAGAAGCC | 858 |
| 132871 | CCCCUCUCACCAGCCUC | 859 |
| 132872 | UUCUCAAGUCUGAAGAA | 860 |
| 132873 | CCCCCUCAUUCUUCAGG | 861 |
| 132874 | GGCUGGGGGGUGUUUGG | 862 |
| 132875 | GGAAAUCCCUAGGAGAC | 863 |
| 132876 | AGAACAAGUGGGUGGCU | 864 |
| 132877 | UAUCAUCUCCAGGGCAG | 865 |
| 132878 | CAGGCCCCUCCCUCCAC | 866 |
| 132879 | CCUGGAGCAGCUGCCUC | 867 |
| 132880 | AGGUUAUGAUGAGGGGU | 868 |
| 132881 | CUGGCUGGGCUGGGCAG | 869 |
| 132882 | CUAGCUGACUGGCUCCC | 870 |
| 132883 | UUCAGACUUGAGAACAA | 871 |
| 132884 | GAGUAAAGGCACAGAAG | 872 |
| 132885 | GGCAAGUGACACCCCUC | 873 |
| 132886 | UGAUGAGGGGUGGGGGG | 874 |
| 132887 | UGGCCCUCUCCAGGCCU | 875 |
| 132888 | UUCAGGUUAUGAUGAGG | 876 |
| 132889 | UAUAUCAUCUCCAGGGC | 877 |
| 132890 | CCCUCCCCAGAGGGCAU | 878 |
| 132891 | CCCCUCUUCAUCCUCCU | 879 |
| 132892 | UCCAGGCUUGCUGGCUG | 880 |
| 132893 | CACACUGGAAUUUCAGG | 881 |
| 132894 | CCUGUCUGGGGUAGGAC | 882 |
| 132895 | GCUCUAGCAAGUGCUUC | 883 |
| 132896 | CUGGCCCUCUCCAGGCC | 884 |
| 132897 | AGACUUGAGAACAAGUG | 885 |
| 132898 | GGAGUAAAGGCACAGAA | 886 |
| 132899 | CCCCUCCCCAGAGGGCA | 887 |
| 132900 | GAGCCACUUCCAGCCCC | 888 |
| 132901 | CUUCCUAGCUGACUGGC | 889 |
| 132902 | CUCCAGCCCUAAGCCUG | 890 |
| 132903 | UGACCUGUUUUAUAUCA | 891 |
| 132904 | CAGCCCCACCCCCUGUG | 892 |
| 132905 | AGGCCCCUCCCUCCACC | 893 |
| 132906 | CUUAGCUCUAGCAAGUG | 894 |
| 132907 | GGGCAAGUGACACCCCU | 895 |
| 132908 | CCCUGUCUGGGGUAGGA | 896 |
| 132909 | GGUGAUUUCUGGCCCUC | 897 |
| 132910 | GGGUGAUUUCUGGCCCU | 898 |
| 132911 | ACACUGGAAUUUCAGGC | 899 |
| 132912 | GACAUAGGCCAGGGGCC | 900 |
| 132913 | AUAUCAUCUCCAGGGCA | 901 |
| 132914 | AUCCUCCUCCCCUCCUC | 902 |
| 143961 | UCCCACUGAUAUUAGAU | 903 |
| 143962 | UGGCCCAUAGCCUCCCU | 904 |
| 143963 | CAGGCAGCUCUGCCACU | 905 |
| 143964 | CAGUAGAAUGGAAUGGG | 906 |
| 143965 | UAUUGGCUCCAGGAUGG | 907 |
| 143966 | CUUCCUCUCCUCCCCAG | 908 |
| 143967 | CAGUCCUGGGUAGGCAU | 909 |
| 143968 | CCUGGAGUAGCUAGCUG | 910 |
| 143969 | CCCAGCUUCUAGCCCCC | 911 |
| 143970 | UCCCUCCAGCUCUUUGU | 912 |
| 143971 | CCUUCCUUCCUCUCCUC | 913 |
| 143972 | CUCGCUAGGACUCAGUU | 914 |
| 143973 | AGAAAUCCCUCUGAGAU | 915 |
| 143974 | GUUUCUUCCCUUCCUUC | 916 |
| 143975 | UUCAGUCCUGGGUAGGC | 917 |
| 143976 | CCCAUGCUUUUCACGGC | 918 |
| 143977 | UUCCCUUCCUUCCUCUC | 919 |
| 143978 | CAUGCCCCCACACUGAC | 920 |
| 143979 | CUUUUCCUCGCUAGGAC | 921 |
| 143980 | CCUCGCUAGGACUCAGU | 922 |
| 143981 | UAUAUUGGCUCCAGGAU | 923 |
| 143982 | GCUCCAGGAUGGGACAG | 924 |
| 143983 | CACGGCCACCUCCGCCA | 925 |
| 143984 | UAGCCCCCCCCACACCA | 926 |
| 143985 | UUUCAGUCCUGGGUAGG | 927 |
| 143986 | GGCCCAUAGCCUCCCUU | 928 |
| 143987 | CUUCCCUUCCUUCCUCU | 929 |
| 143988 | GACCUCAGGCCUGCUUU | 930 |
| 143989 | CCCCAGCUUCUAGCCCC | 931 |
| 143990 | UUUCUUCCCUUCCUUCC | 932 |
| 143991 | UAGGGAUAAAACUGAGC | 933 |
| 143992 | AUAUUGGCUCCAGGAUG | 934 |
| 143993 | ACGGCCACCUCCGCCAC | 935 |
| 143994 | ACAGCCUAGAGCCAGUG | 936 |
| 143995 | ACAGAAGCCACCUGAAA | 937 |
| 143996 | UCAGUCCUGGGUAGGCA | 938 |
| 143997 | UCACGGCCACCUCCGCC | 939 |
| 143998 | UCCUCGCUAGGACUCAG | 940 |
| 143999 | CUCCCACUGAUAUUAGA | 94 |
| 144000 | UUUUCCUCGCUAGGACU | 942 |
| 144001 | UGUGGGCUAGAUGGCUG | 943 |
| 144002 | GCCCAUAGCCUCCCUUU | 944 |
| 144003 | UAAUAGCUCAGAGCAAG | 945 |
| 144004 | AGUCCUGGGUAGGCAUG | 946 |
| 144005 | CAGCCUAGAGCCAGUGA | 947 |
| 144006 | CUCUCCUCCCCAGGGGC | 948 |
| 144007 | GAUAGAGAACUACAGUA | 949 |
| 144008 | GUGGCGGAGGUGGCCGU | 950 |
| 144009 | GGUCAUGCUGUCCCUUG | 951 |
| 144010 | GGUUCCUGGUGUGGGGG | 952 |
| 144011 | UCAGCAUAUUAGAGUAG | 953 |
| 144012 | UAUCCCUAGAAGCAGCU | 954 |
| 144013 | UCUAUCUAAUAUCAGUG | 955 |
| 144014 | CUAUACUCCACCUUCCA | 956 |
| 144015 | CCCAUUCCAUUCUACUG | 957 |
| 144016 | CUCCGUUGCUCCACAGU | 958 |
| 144017 | UCCCCUGUCUUUUCCUG | 959 |
| 144018 | AUCCCUAGAAGCAGCUA | 960 |
| 144019 | CACCCCACUUGGGGGGC | 961 |
| 144020 | UUUCUCAGCAUAUUAGA | 962 |
| 144021 | CAGGUGGCUUCUGUGAA | 963 |
| 144022 | CCCAGCUCACUGGGCCU | 964 |
| 144023 | CUCAGCAUAUUAGAGUA | 965 |
| 144024 | CAUUCUACUGGAAGGCU | 966 |
| 144025 | UCCUGUCUCACCGACCU | 967 |
| 144026 | GUAUCCAUGCCUACCCA | 968 |
| 144027 | AGGUGGCUUCUGUGAAG | 969 |
| 144028 | GGAUCACAGGUGGAGGU | 970 |
| 144029 | UUUAGCUUGCUCUGAGC | 971 |
| 144030 | GAAGCCUUUGGUAUCCA | 972 |
| 144031 | CUGUCUCACCGACCUCA | 973 |
| 144032 | UGUGAAGGAGCCUGUCA | 974 |
| 144033 | ACUCUGCCCCCUCCCAC | 975 |
| 144034 | UCUCACUAAUCCCUGCC | 976 |
| 144035 | UACUGGAAGGCUUUCAG | 977 |
| 144036 | UAUACUCCACCUUCCAC | 978 |
| 144037 | GCCCAGCUCACUGGGCC | 979 |
| 144038 | CUCUGAGCUAUUAGAAG | 980 |
| 144039 | GGGGGCUGGGUCUACUG | 981 |
| 144040 | GGAUUCAUGACCCAGGA | 982 |
| 144041 | CCUGCUCAGUUUUAUCC | 983 |
| 144042 | CUCAACUCCUCUGGCAG | 984 |
| 144043 | CUGCCUCAGGCUCUGGU | 985 |
| 144044 | UGUGCCCGCUGUCCCAU | 986 |
| 144045 | UCCUUCUCUCACUAAUC | 987 |
| 144046 | GCUUGCUCUGAGCUAUU | 988 |
| 144047 | UCAACUCCUCUGGCAGA | 989 |
| 144048 | CCUGUCUCACCGACCUC | 990 |
| 144049 | CUCCACAGUGGCACCAC | 991 |
| 144050 | UCCCUAGAAGCAGCUAG | 992 |
| 144051 | GGACUCAUGGUCUCCAC | 993 |
| 144052 | AGCUUGCUCUGAGCUAU | 994 |
| 144053 | GGUAUCCAUGCCUACCC | 995 |
| 144054 | CUGGUGUGGGGGGGGCU | 996 |
| 144055 | CACUGGGUAGUGGCAGA | 997 |
| 144056 | UGCAGAGUAUUUCUAUA | 998 |
| 144057 | GAGUAGAUGUCCCGUUC | 999 |

TABLE 2
Exemplary Spacer Sequences Targeting APOC3 for
CasM.265466 Effector Proteins

| | Spacer sequence (5′ to 3′), | SEQ |
|---|---|---|
| Spacer ID | shown as RNA | ID NO: |
| 127937 | CCUAGAGGCAGCUGCUCCAG | 209 |
| 127938 | CAUCCCUAGAGGCAGCUGCU | 210 |
| 127939 | AUCCCUAGAGGCAGCUGCUC | 211 |
| 125647 | GGUUGCUUAAAAGGGACAGU | 212 |
| 125662 | AGCAACCUACAGGGGCAGCC | 213 |
| 125674 | CAGCCCCGGGUACUCCUUGU | 214 |
| 127961 | CCAGGUGGCCCAGCAGGCCA | 215 |
| 127965 | CAGGAGUCCCAGGUGGCCCA | 216 |
| 127970 | AGUGCAUCCUUGGCGGUCUU | 217 |
| 125678 | CUGGGCCACCUGGGACUCCU | 218 |
| 125679 | CAUCCUUGGCGGUCUUGGUG | 219 |
| 125682 | GGUGACCGAUGGCUUCAGUU | 220 |
| 125683 | CCCCUGUAGGUUGCUUAAAA | 221 |
| 125687 | GAGCACCGUUAAGGACAAGU | 222 |
| 125688 | GCUUCAGUUCCCUGAAAGAC | 223 |
| 125694 | AGACCUCAAUACCCCAAGUC | 224 |
| 125696 | CUUAAAAGGGACAGUAUUCU | 225 |
| 125697 | AAGCUGGACAAGAAGCUGCU | 226 |
| 125699 | CCUGAGACCUCAAUACCCCA | 227 |
| 125704 | ACCGAUGGCUUCAGUUCCCU | 228 |
| 125709 | AAAGGGACAGUAUUCUCAGU | 229 |
| 125710 | AAGCCAUCGGUCACCCAGCC | 230 |
| 125714 | UCCCUUUUAAGCAACCUACA | 231 |
| 125715 | UCCUUAACGGUGCUCCAGUA | 232 |
| 125716 | AGAAUACUGUCCCUUUUAAG | 233 |
| 125718 | CCUGGAGGGGGGCCAGGCAU | 234 |
| 125722 | ACGGUGCUCCAGUAGUCUUU | 235 |
| 125723 | GCAGCUUCUUGUCCAGCUUU | 236 |
| 125724 | GUCUUUCAGGGAACUGAAGC | 237 |
| 125727 | GGGUAUUGAGGUCUCAGGCA | 238 |
| 125729 | CUCCAGUAGUCUUUCAGGGA | 239 |
| 125734 | GACUUGGGGUAUUGAGGUCU | 240 |
| 125738 | CUGUCCCUUUUAAGCAACCU | 241 |
| 127996 | CCCUGAAAGACUACUGGAGC | 242 |
| 128004 | UAGGUUGCUUAAAAGGGACA | 243 |
| 128006 | CCUGAAAGACUACUGGAGCA | 244 |
| 128011 | CUGGAGCACCGUUAAGGACA | 245 |
| 128016 | AAAGACUACUGGAGCACCGU | 246 |
| 128021 | GCUUAAAAGGGACAGUAUUC | 247 |
| 128025 | AGUUCCCUGAAAGACUACUG | 248 |
| 128032 | CAGUUCCCUGAAAGACUACU | 249 |
| 128035 | AAUACCCCAAGUCCACCUGC | 250 |
| 128036 | AUGCCUGGCCCCCCUCCAGG | 251 |
| 128041 | CAGGGCUGCCCCUGUAGGUU | 252 |
| 128042 | AAAAGGGACAGUAUUCUCAG | 253 |
| 128047 | CCAAUAAAGCUGGACAAGAA | 254 |
| 128076 | AGGGAACUGAAGCCAUCGGU | 255 |
| 128080 | UUAAGCAACCUACAGGGGCA | 256 |
| 128081 | CUUGUCCAGCUUUAUUGGGA | 257 |
| 128085 | CCUUUUAAGCAACCUACAGG | 258 |
| 128100 | UUUCAGGGAACUGAAGCCAU | 259 |
| 128109 | CUUAACGGUGCUCCAGUAGU | 260 |
| 128110 | CAGUAGUCUUUCAGGGAACU | 261 |
| 128111 | UCAGGGAACUGAAGCCAUCG | 262 |
| 128112 | AACGGUGCUCCAGUAGUCUU | 263 |
| 128113 | UAAGCAACCUACAGGGGCAG | 264 |
| 128115 | UUGUCCAGCUUUAUUGGGAG | 265 |
| 128116 | GGGGUAUUGAGGUCUCAGGC | 266 |
| 128117 | CAGGGAACUGAAGCCAUCGG | 267 |
| 128118 | GUCCUUAACGGUGCUCCAGU | 268 |
| 128120 | AAGCAACCUACAGGGGCAGC | 269 |
| 127514 | GAGCAGCUGCCUCUAGGGAU | 270 |
| 127515 | CCUGGAGCAGCUGCCUCUAG | 271 |
| 128121 | ACCUGGAGCAGCUGCCUCUA | 272 |
| 128122 | CCCAGAGGGCAUUACCUGGA | 273 |
| 128123 | CCCUCCCCAGAGGGCAUUAC | 274 |
| 128124 | CCCCUCCCCAGAGGGCAUUA | 275 |
| 128125 | UCCCCUCCCCAGAGGGCAUU | 276 |
| 128126 | UUUCCCCUCCCCAGAGGGCA | 277 |
| 128127 | CUCUUUCCCCUCCCCAGAGG | 278 |
| 128128 | CCCUCCUCUUUCCCCUCCCC | 279 |
| 128129 | CUCCCCUCCUCUUUCCCCUC | 280 |
| 127516 | CUCUUUCCUCAGGAGCUUCA | 281 |
| 127517 | AAGCUCCUGAGGAAAGAGCA | 282 |
| 128130 | AGCCCUGCUCUUUCCUCAGG | 283 |
| 128131 | UUUCCUCAGGAGCUUCAGAG | 284 |
| 128132 | UCCUCAGGAGCUUCAGAGGC | 285 |
| 128133 | CCUCAGGAGCUUCAGAGGCC | 286 |
| 128134 | CUCAGGAGCUUCAGAGGCCG | 287 |
| 128135 | AGGAGCUUCAGAGGCCGAGG | 288 |
| 128136 | UGAAGCUCCUGAGGAAAGAG | 289 |
| 128137 | GGCCUCUGAAGCUCCUGAGG | 290 |
| 127518 | GCCUGCUGGGCCACCUGGGA | 292 |
| 127519 | CCUGGCCUGCUGGGCCACCU | 293 |
| 127520 | UACCUGGCCUGCUGGGCCAC | 294 |
| 127521 | GGGAGGGAGGCCAGCGGGUG | 295 |
| 127522 | CCCCCAGCCCAGUCCCACCA | 296 |
| 128138 | CCCUGCUCUGUUGCUUCCCC | 297 |
| 88586 | GCCUCAGGGUUCAAAUCCCA | 298 |
| 88592 | GCCCUGCAUGAAGCCAAGAA | 299 |
| n/a | AGUUCUGGGAUUUGGACCCU | 823 |
| n/a | GACCCUGAGGUCAGACCAAC | 824 |
| n/a | ACCUCAGGGUCCAAAUCCCA | 825 |
| 133653 | GACAGCCCAGUCCUACCCCA | 1000 |
| 133654 | UUCAGGGCUUGGGGCUGGUG | 1001 |
| 133655 | CCUUUACUCCAAACACCCCC | 1002 |
| 133656 | CCCCCCACCCCUCAUCAUAA | 1003 |
| 133657 | UUCAGUCUGGUGGGUUUUCU | 1004 |
| 133658 | UCACUUGCCCAAAGCUACAC | 1005 |
| 133659 | GUGGGUUUUCUGCUCCAUCC | 1006 |
| 133660 | CCGGAGCCACUGAUGCCUGG | 1007 |
| 133661 | GACUCAGUCUCCUAGGGAUU | 1008 |
| 133662 | GCCUAUGUCCAAGCCAUUUC | 1009 |
| 133663 | CCUCAGGCCCUCAUCUCCAC | 1010 |
| 133664 | UCCAAGCCAUUUCCCCUCUC | 1011 |
| 133665 | AAAGGCUGAGAUGGGCCCGA | 1012 |
| 133666 | AAAUUCCAGUGUGAAAGGCU | 1013 |
| 133667 | GAGAUGAUAUAAAACAGGUC | 1014 |
| 133668 | GGGAGGGGAAAGAGGAGGGG | 1015 |
| 133669 | AAGAACAUGGAGGCCCGGGA | 1016 |
| 133670 | GGGCUGGUGGAGGGAGGGGC | 1017 |
| 133671 | AUGCCUGGUCUUCUGUGCCU | 1018 |
| 133672 | UGUUCAGGGCUUGGGGCUGG | 1019 |
| 133673 | CUCCAGGUAAUGCCCUCUGG | 1020 |
| 133674 | AGGGCUCCCCAGGCCCACCC | 1021 |
| 133675 | UUGGCUGGACUGGACGGAGA | 1022 |
| 133676 | GAGCUAAGGAAGCCUCGGAG | 1023 |
| 133677 | CCCUCUGGGGAGGGGAAAGA | 1024 |
| 133678 | CUCCAAACACCCCCCAGCCC | 1025 |
| 133679 | GGGAGCCAGUCAGCUAGGAA | 1026 |
| 133680 | GGCCCGAGGCCCCUGGCCUA | 1027 |
| 133681 | GAGGCAGCUGCUCCAGGUAA | 1028 |
| 133682 | GAGGGAGGGGCCUGAAAUUC | 1029 |
| 133683 | GAGAGGGCCAGAAAUCACCC | 1030 |
| 133684 | AAGAAGCCCCUCACCCCUCU | 1031 |
| 133685 | CCCCAGACAGGGAAACUGAG | 1032 |
| 133686 | GGGCUGGAAGUGGCUCCAAG | 1033 |
| 133687 | CUCCAGGCUGUGUUCAGGGC | 1034 |
| 133688 | GUCUUCUGUGCCUUUACUCC | 1035 |
| 133689 | CACAGGGGGUGGGGCUGGAA | 1036 |
| 133690 | GACUGGACGGAGAUCAGUCC | 1037 |
| 133691 | GACGGGUGCCCCCCACCCCU | 1038 |
| 133692 | UGUCCAAGCCAUUUCCCCUC | 1039 |
| 133693 | AGAUGGGCCCGAGGCCCCUG | 1040 |
| 133694 | GGUCCUCAGUGCCUGCUGCC | 1041 |
| 133695 | CAGGGCUGGCGGGACAGCAG | 1042 |
| 133696 | CCUUGAUGUUCAGUCUGGUG | 1043 |
| 133697 | AGGCCUGGAGAGGGCCAGAA | 1044 |
| 133698 | CCCUGGAGAUGAUAUAAAAC | 1045 |
| 133699 | ACCUGAAGAACAUGGAGGCC | 1046 |
| 133700 | CCUGGUCUUCUGUGCCUUUA | 1047 |
| 133701 | ACCUUUGCCCAGCGCCCUGG | 1048 |
| 133702 | CCCAAAGCUACACAGGGGGU | 1049 |
| 133703 | GUGGAGGGAGGGGCCUGAAA | 1050 |
| 133704 | UGCCUUUACUCCAAACACCC | 1051 |
| 133705 | CUCCAUCCCACCCACCUCCC | 1052 |
| 133706 | AUGUUCAGUCUGGUGGGUUU | 1053 |
| 133707 | CUAGAGCUAAGGAAGCCUCG | 1054 |
| 133708 | GGAAGGAAUGAGGGCUCCCC | 1055 |
| 133709 | GGCUGCAGGGCUGGCGGGAC | 1056 |
| 133710 | AUGCCCUCUGGGGAGGGGAA | 1057 |
| 133711 | AAACAGGUCAGAACCCUCCU | 1058 |
| 133712 | GGGCUGGAGGAAGCCUUAGA | 1059 |
| 133713 | UUCUCAAGUCUGAAGAAGCC | 1060 |
| 133714 | AGCUCAUCUGGGCUGCAGGG | 1061 |
| 133715 | GGGAUUUCCCAACUCUCCCG | 1062 |
| 133716 | GACGGAGAUCAGUCCAGACC | 1063 |
| 133717 | UGAAAGGCUGAGAUGGGCCC | 1064 |
| 133718 | CCUGUCUGCUCAGUUCAUCC | 1065 |
| 133719 | CAGGUUCCCCCCUCAUUCUU | 1066 |
| 133720 | GUCAGCAGGUGACCUUUGCC | 1067 |
| 133721 | GAGGAAGCCUUAGACAGCCC | 1068 |
| 133722 | CUGCAUCUGGACACCCUGCC | 1069 |
| 133723 | CCCAGCGCCCUGGGUCCUCA | 1070 |
| 133724 | CCCAGCCCAGCCAGCAAGCC | 1071 |
| 133725 | GGUUUUCUGCUCCAUCCCAC | 1072 |
| 133726 | UAAAACAGGUCAGAACCCUC | 1073 |
| 133727 | CUCAGUUCAUCCCUAGAGGC | 1074 |
| 133728 | GACACCCUGCCUCAGGCCCU | 1075 |
| 133729 | GCGGGACAGCAGCGUGGACU | 1076 |
| 133730 | AAGAGGGGCAAGAGGAGCUC | 1077 |
| 133731 | CAUCUGGACACCCUGCCUCA | 1078 |
| 133732 | GAGGCCCGGGAGGGGUGUCA | 1079 |
| 133733 | CCUGCUGCCCUGGAGAUGAU | 1080 |
| 133734 | AGGAAGCCUCGGAGCUGGAC | 1081 |
| 133735 | UCUGCUCAGUUCAUCCCUAG | 1082 |
| 133736 | GAAGUGGCUCCAAGUGCAGG | 1083 |
| 133737 | GCUGGACUGGACGGAGAUCA | 1084 |
| 133738 | GAGAAGCACUUGCUAGAGCU | 1085 |
| 133739 | CUGCCCUGGAGAUGAUAUAA | 1086 |
| 133740 | AUAUAAAACAGGUCAGAACC | 1087 |
| 133741 | GCUCCAAGUGCAGGUUCCCC | 1088 |
| 133742 | GGCUGGGCAGGGAGCUCCUC | 1089 |
| 133743 | GACAUAGGCCAGGGGCCUCG | 1090 |
| 133744 | GGCCUGGGGAGCCCUCAUUC | 1091 |
| 133745 | GGCUGGGGGGUGUUUGGAGU | 1092 |
| 133746 | GGUGGGAUGGAGCAGAAAAC | 1093 |
| 133747 | GCUUGGACAUAGGCCAGGGG | 1094 |
| 133748 | AGGGCCUGAGGCAGGGUGUC | 1095 |
| 133749 | AGGCAGGGUGUCCAGAUGCA | 1096 |
| 133750 | UCCCGCCAGCCCUGCAGCCC | 1097 |
| 133751 | CCCCUCUUCAUCCUCCUCCC | 1098 |
| 133752 | AACAUCAAGGCACCUGCGGU | 1099 |
| 133753 | AGCUCAGGAACUGGGGGUGG | 1100 |
| 133754 | UUCUUCAGGUUAUGAUGAGG | 1101 |
| 133755 | GCUUUGGGCAAGUGACACCC | 1102 |
| 133756 | AGGGGGGAACCUGCACUUGG | 1103 |
| 133757 | GCUUGGGCUGGGGGGUGUUU | 1104 |
| 133758 | AACUGAGCAGACAGGCAGGA | 1105 |
| 133759 | UGUGUCUUUGGGUGAUUUCU | 1106 |
| 133760 | UGAUGAGGGGUGGGGGGCAC | 1107 |
| 133761 | GGCAGGGAGCUCCUCUUGCC | 1108 |
| 133762 | AGGGGUGGGGGGCACCCGUC | 1109 |
| 133763 | CCUGGAGCAGCUGCCUCUAG | 1110 |
| 133764 | GGGAUGAACUGAGCAGACAG | 1111 |
| 133765 | AGGCUUCCUCCAGCCCUAAG | 1112 |
| 133766 | GAGAUGAGGGCCUGAGGCAG | 1113 |
| 133767 | GGAUGGAGCAGAAAACCCAC | 1114 |
| 133768 | AAGAAUGAGGGGGGAACCUG | 1115 |
| 133769 | UUUGGAGUAAAGGCACAGAA | 1116 |
| 133770 | GAGUAGAGGGGUGAGGGGCU | 1117 |
| 133771 | ACCUGUUUUAUAUCAUCUCC | 1118 |
| 133772 | GUGAGAGGGGAAAUGGCUUG | 1119 |
| 133773 | UGUAGCUUUGGGCAAGUGAC | 1120 |
| 133774 | UGUCUUUGGGUGAUUUCUGG | 1121 |
| 133775 | UAUCAUCUCCAGGGCAGCAG | 1122 |
| 133776 | GAGCAGAAAACCCACCAGAC | 1123 |
| 133777 | GGAGACUGAGUCCACGCUGC | 1124 |
| 133778 | CAGCCCAGAUGAGCUCAGGA | 1125 |
| 133779 | GGUGGCUUGGGCUGGGGGGU | 1126 |
| 133780 | AGGGGCUUCUUCAGACUUGA | 1127 |
| 133781 | CACUUGGAGCCACUUCCAGC | 1128 |
| 133782 | CAGCAAGCGGGCGGGAGAGU | 1129 |
| 133783 | GGCAAAGGUCACCUGCUGAC | 1130 |
| 133784 | UCUUUGGGUGAUUUCUGGCC | 1131 |
| 133785 | UCAUCUCCAGGGCAGCAGGC | 1132 |
| 133786 | AGCAGACAGGCAGGAGGGUU | 1133 |
| 133787 | AGGACCCAGGGCGCUGGGCA | 1134 |
| 133788 | GGGGGUGUUUGGAGUAAAGG | 1135 |
| 133789 | GGGUAGGACUGGGCUGUCUA | 1136 |
| 133790 | GGUGAUUUCUGGCCCUCUCC | 1137 |
| 133791 | GAGCAGCUGCCUCUAGGGAU | 1138 |
| 133792 | CUGUGUGUCUUUGGGUGAUU | 1139 |
| 133793 | CUGACCAGUGGAGAUGAGGG | 1140 |
| 133794 | AUGAGGGGUGGGGGGCACCC | 1141 |
| 133795 | UCUAAGGCUUCCUCCAGCCC | 1142 |
| 133796 | GAAUUUCAGGCCCCUCCCUC | 1143 |
| 133797 | AGCCUGAAGAAUGAGGGGGG | 1144 |
| 133798 | GGGGGCACCCGUCCAGCUCC | 1145 |
| 133799 | AAGGCACAGAAGACCAGGCA | 1146 |
| 133800 | ACCAGUGGAGAUGAGGGCCU | 1147 |
| 133801 | GGCUGUCUAAGGCUUCCUCC | 1148 |
| 133802 | GGGGUGGGCCUGGGGAGCCC | 1149 |
| 133803 | UAGCUUUGGGCAAGUGACAC | 1150 |
| 133804 | GAGCCACUUCCAGCCCCACC | 1151 |
| 133805 | AUUUCUGGCCCUCUCCAGGC | 1152 |
| 133806 | ACUGGCUCCCCAGGGAGAGG | 1153 |
| 133807 | GCCCUCUCCAGGCCUCAGUU | 1154 |
| 133808 | GGACUGGGCUGUCUAAGGCU | 1155 |
| 133809 | CUGUCCCGCCAGCCCUGCAG | 1156 |
| 133810 | GGCAAGUGACACCCCUCCCG | 1157 |
| 133811 | AGAACAAGUGGGUGGCUUGG | 1158 |
| 133812 | AACACAGCCUGGAGUAGAGG | 1159 |
| 133813 | UCUGGGGUAGGACUGGGCUG | 1160 |
| 133814 | GAGGGGUGAGGGGCUUCUUC | 1161 |
| 133815 | CGGUCUGGACUGAUCUCCGU | 1162 |
| 133816 | GCUGGGCUGGGCAGGGAGCU | 1163 |
| 133817 | GGGAGCCCUCAUUCCUUCCU | 1164 |
| 133818 | GAGUAAAGGCACAGAAGACC | 1165 |
| 133819 | GCAAGUGCUUCUCCAGGCUU | 1166 |
| 133820 | AGAGGGGAAAUGGCUUGGAC | 1167 |
| 133821 | AGUCCACGCUGCUGUCCCGC | 1168 |
| 133822 | CCUCUAGGGAUGAACUGAGC | 1169 |
| 133823 | GGCCAGGGGCCUCGGGCCCA | 1170 |
| 133824 | CUGGCUGGGCUGGGCAGGGA | 1171 |
| 133825 | AUCUCCGUCCAGUCCAGCCA | 1172 |
| 133826 | GACUGAUCUCCGUCCAGUCC | 1173 |
| 133827 | GGAAAUCCCUAGGAGACUGA | 1174 |
| 133828 | GCUGACUGGCUCCCCAGGGA | 1175 |
| 133829 | ACACCCCUCCCGGGCCUCCA | 1176 |
| 133830 | GCUCUAGCAAGUGCUUCUCC | 1177 |
| 133831 | CUUCUCCAGGCUUGCUGGCU | 1178 |
| 133832 | UUUUAUAUCAUCUCCAGGGC | 1179 |
| 133833 | UCCAGAUGCAGCAAGCGGGC | 1180 |
| 133834 | GCUCCCCAGGGAGAGGCUGG | 1181 |
| 144542 | ACAGGCUCCUUCACAGAAGC | 1182 |
| 144543 | CUCUGCAGAACGGGACAUCU | 1183 |
| 144544 | GCCCCCCCCACACCAGGAAC | 1184 |
| 144545 | AUAUGCUGAGAAACAAUAGG | 1185 |
| 144546 | GGCAGGGAUUAGUGAGAGAA | 1186 |
| 144547 | GACCUCAGGCCUGCUUUACA | 1187 |
| 144548 | CAGAACGGGACAUCUACUCU | 1188 |
| 144549 | GAGUAGCUAGCUGCUUCUAG | 1189 |
| 144550 | GUGCCACUGUGGAGCAACGG | 1190 |
| 144551 | GGGAAUCUGUGGUGCCACUG | 1191 |
| 144552 | GUGAGAGCUUCUCCCUCCAG | 1192 |
| 144553 | GCUCCAGGAUGGGACAGCGG | 1193 |
| 144554 | CUGAGAAACAAUAGGUUUCU | 1194 |
| 144555 | ACCUGUUUUAUAUUGGCUCC | 1195 |
| 144556 | AGAUUGCCCAUGCUUUUCAC | 1196 |
| 144557 | GGGUGGAAGGUGGAGUAUAG | 1197 |
| 144558 | CCAAAGGCUUCUAAUAGCUC | 1198 |
| 144559 | AGACAGGAAAAGACAGGGGA | 1199 |
| 144560 | GACCCAGCCCCCCAAGUGGG | 1200 |
| 144561 | CCGUAGCUGGGCAGGGAUUA | 1201 |
| 144562 | CAGUAGACCCAGCCCCCCAA | 1202 |
| 144563 | GGUGGUGAGAGCUUCUCCCU | 1203 |
| 144564 | GAAUGGAAUGGGGAAUCUGU | 1204 |
| 144565 | GAUGGCUGGGUGGUGAGAGC | 1205 |
| 144566 | UGGAGCAACGGAGGAAGUGG | 1206 |
| 144567 | GCCUCCCUUUCCCCAGCUUC | 1207 |
| 144568 | GGUCAUGAAUCCCAAGCCUU | 1208 |
| 144569 | GCUAGCUGCUUCUAGGGAUA | 1209 |
| 144570 | GCCCAUAGCCUCCCUUUCCC | 1210 |
| 144571 | GAGACCAUGAGUCCCAAGCC | 1211 |
| 144572 | UGACCUGUUUUAUAUUGGCU | 1212 |
| 144573 | CUCUAAUAUGCUGAGAAACA | 1213 |
| 144574 | CCACUGUGGAGCAACGGAGG | 1214 |
| 144575 | ACCUCCACCUGUGAUCCCAA | 1215 |
| 144576 | CUUUACAGCCUAGAGCCAGU | 1216 |
| 144577 | CUGAGCAGUCCAGACCAGAG | 1217 |
| 144578 | AGGCAGGAAGGCCAUGCAGC | 1218 |
| 144579 | UUGGCUCCAGGAUGGGACAG | 1219 |
| 144580 | AAAGCCUUCCAGUAGAAUGG | 1220 |
| 144581 | AUAUUAGAUAGAGAACUACA | 1221 |
| 144582 | CCCAGUGCAAGGCUUUUGGC | 1222 |
| 144583 | UAGAAAUACUCUGCAGAACG | 1223 |
| 144584 | AAUCCCAAGCCUUUCUCCCA | 1224 |
| 144585 | GAUACCAAAGGCUUCUAAUA | 1225 |
| 144586 | AAACUGAGCAGGCAAGCGGG | 1226 |
| 144587 | CUUUUCACGGCCACCUCCGC | 1227 |
| 144588 | AGAAAUCCCUCUGAGAUUGC | 1228 |
| 144589 | GAGUAUAGAAAUACUCUGCA | 1229 |
| 144590 | AAGGUUACAUGCCCCCACAC | 1230 |
| 144591 | UUUCUUCCCUUCCUUCCUCU | 1231 |
| 144592 | UUUUAUAUUGGCUCCAGGAU | 1232 |
| 144593 | GAGCCAGUGACAGGCUCCUU | 1233 |
| 144594 | GAAGGUGGAGUAUAGAAAUA | 1234 |
| 144595 | CCACUACCCAGUGCAAGGCU | 1235 |
| 144596 | GGUUUCUUUUCCUCGCUAGG | 1236 |
| 144597 | AGGUCGGUGAGACAGGAAAA | 1237 |
| 144598 | GAGAACUACAGUAGACCCAG | 1238 |
| 144599 | AGCUGGGCAAAGGUCACCUG | 1239 |
| 144600 | UUAGAUAGAGAACUACAGUA | 1240 |
| 144601 | CAGGCAGCUCUGCCACUACC | 1241 |
| 144602 | CAAGGCUUUUGGCCCAUAGC | 1242 |
| 144603 | AGAGCUUCUCCCUCCAGCUC | 1243 |
| 144604 | GGUAGGCAUGGAUACCAAAG | 1244 |
| 144605 | AGAAACAAUAGGUUUCUUUU | 1245 |
| 144606 | GGGUGGGAGGGGGCAGAGUG | 1246 |
| 144607 | UGCUGAGAAACAAUAGGUUU | 1247 |
| 144608 | GCUGGGCAGGGAUUAGUGAG | 1248 |
| 144609 | AACAAGGGACAGCAUGACCC | 1249 |
| 144610 | GAGCAACGGAGGAAGUGGGG | 1250 |
| 144611 | CCGGCUCACCUAGAUGAGGU | 1251 |
| 144612 | GGACUCAGUUUUUUCAGUCC | 1252 |
| 144613 | GAAAUACUCUGCAGAACGGG | 1253 |
| 144614 | GGCUAGAUGGCUGGGUGGUG | 1254 |
| 144615 | GGAGGGGGCAGAGUGAAGGU | 1255 |
| 144616 | GCUGCUUCUAGGGAUAAAAC | 1256 |
| 144617 | CCUGGAGUAGCUAGCUGCUU | 1257 |
| 144618 | CCAGAGGAGUUGAGAAAUCC | 1258 |
| 144619 | CAGCCAUCUGCCAGAGGAGU | 1259 |
| 144620 | CCCCCACACUGACCUCCACC | 1260 |
| 144621 | GAUAGAGAACUACAGUAGAC | 1261 |
| 144622 | CAUGCCCCCACACUGACCUC | 1262 |
| 144623 | UGAUCCCAACAGUCUCCUCU | 1263 |
| 144624 | AUCCCAACAGUCUCCUCUGC | 1264 |
| 144625 | GAAUGGGGAAUCUGUGGUGC | 1265 |
| 144626 | GCUGGGUGGUGAGAGCUUCU | 1266 |
| 144627 | ACCCAAUUGCAGGCAGCUCU | 1267 |
| 144628 | GGACAGCGGGCACAGAAGGC | 1268 |
| 144629 | GAUGAGGUCGGUGAGACAGG | 1269 |
| 144630 | UGGUGCCACUGUGGAGCAAC | 1270 |
| 144631 | GGCAUGGAUACCAAAGGCUU | 1271 |
| 144632 | AGCAGGCAAGCGGGGAGGGC | 1272 |
| 144633 | AGUCCCAAGCCUUCUGUGGG | 1273 |
| 144634 | AUAGCUCAGAGCAAGCUAAA | 1274 |
| 144635 | GGGAUAAAACUGAGCAGGCA | 1275 |
| 144636 | AGCAGUCCAGACCAGAGCCU | 1276 |
| 144637 | UGGGCUAGAUGGCUGGGUGG | 1277 |
| 144638 | GCUCAGAGCAAGCUAAACAA | 1278 |
| 144639 | CUUCUAGGGAUAAAACUGAG | 1279 |
| 144640 | CCCAUGCUUUUCACGGCCAC | 1280 |
| 144641 | UAUUGGCUCCAGGAUGGGAC | 1281 |
| 144642 | GGCAAAGGUCACCUGCUGAG | 1282 |
| 144643 | CAGCCUAGAGCCAGUGACAG | 1283 |
| 144644 | AAAAGCAUGGGCAAUCUCAG | 1284 |
| 144645 | CUCCAGGUAAUGCCCCUGGG | 1285 |
| 144646 | GCUACUCCAGGUAAUGCCCC | 1286 |
| 144647 | UCCCCUGUCUUUUCCUGUCU | 1287 |
| 144648 | UGCCCGCUGUCCCAUCCUGG | 1288 |
| 144649 | CUCAGUUUUAUCCCUAGAAG | 1289 |
| 144650 | CAGAGUAUUUCUAUACUCCA | 1290 |
| 144651 | CUGUCCCUUGUUUAGCUUGC | 1291 |
| 144652 | GGUGAGCCGGUAGCUGAUCC | 1292 |
| 144653 | CUGGAAGGCUUUCAGGUGGC | 1293 |
| 144654 | GGCCAAAAGCCUUGCACUGG | 1294 |
| 144655 | CCCCUGGGGAGGAGAGGAAG | 1295 |
| 144656 | CUGUAGUUCUCUAUCUAAUA | 1296 |
| 144657 | GGUCUACUGUAGUUCUCUAU | 1297 |
| 144658 | UCUAAUAUCAGUGGGAGAAA | 1298 |
| 144659 | AUCCCUUGGUGGCGGAGGUG | 1299 |
| 144660 | GAUGUCCCGUUCUGCAGAGU | 1300 |
| 144661 | CCUGCUCAGUUUUAUCCCUA | 1301 |
| 144662 | AAACAGGUCACAGCCCUCCC | 1302 |
| 144663 | UCACUGGCUCUAGGCUGUAA | 1303 |
| 144664 | CUCUGAGCUAUUAGAAGCCU | 1304 |
| 144665 | AUCCCUGCCCAGCUACGGCA | 1305 |
| 144666 | GCUUCUGUGAAGGAGCCUGU | 1306 |
| 144667 | UAGUUCUCUAUCUAAUAUCA | 1307 |
| 144668 | CGGCAGAGGAGACUGUUGGG | 1308 |
| 144669 | AAGGAGCCUGUCACUGGCUC | 1309 |
| 144670 | GCCCACAGAAGGCUUGGGAC | 1310 |
| 144671 | UCCCUAGAAGCAGCUAGCUA | 1311 |
| 144672 | CCUACCCAGGACUGAAAAAA | 1312 |
| 144673 | UUGUUUCUCAGCAUAUUAGA | 1313 |
| 144674 | UUGGGAUCACAGGUGGAGGU | 1314 |
| 144675 | GCAGAUGGCUGCAUGGCCUU | 1315 |
| 144676 | CCUCAGGCUCUGGUCUGGAC | 1316 |
| 144677 | ACCUUUGCCCAGCUCACUGG | 1317 |
| 144678 | GUAUCCAUGCCUACCCAGGA | 1318 |
| 144679 | GAAGCCUUUGGUAUCCAUGC | 1319 |
| 144680 | GGGGCAUGUAACCUUCACUC | 1320 |
| 144681 | GGGAAAGGGAGGCUAUGGGC | 1321 |
| 144682 | UGAAGGAGCCUGUCACUGGC | 1322 |
| 144683 | GGGGGGGCUAGAAGCUGGGG | 1323 |
| 144684 | GGUAGUGGCAGAGCUGCCUG | 1324 |
| 144685 | UCCCAUCCUGGAGCCAAUAU | 1325 |
| 144686 | GAAGCUGGGGAAAGGGAGGC | 1326 |
| 144687 | GCUGAUCCCUUGGUGGCGGA | 1327 |
| 144688 | GCGAGGAAAAGAAACCUAUU | 1328 |
| 144689 | GACUGCUCAGCAGGUGACCU | 1329 |
| 144690 | GAAGGCUUUCAGGUGGCUUC | 1330 |
| 144691 | AGGUCCAAGGCUUGUCCCCU | 1331 |
| 144692 | UGGGGGGGGCUAGAAGCUGG | 1332 |
| 144693 | UUUCUCAGCAUAUUAGAGUA | 1333 |
| 144694 | GGCAAUCUCAGAGGGAUUUC | 1334 |
| 144695 | AAGCAGGCCUGAGGUCCAAG | 1335 |
| 144696 | UCUCACCGACCUCAUCUAGG | 1336 |
| 144697 | UCCCGUUCUGCAGAGUAUUU | 1337 |
| 144698 | UCAGUGGGAGAAAGGCUUGG | 1338 |
| 144699 | GAAGCAGCUAGCUACUCCAG | 1339 |
| 144700 | AUAUCAGUGGGAGAAAGGCU | 1340 |
| 144701 | AUGCCCCUGGGGAGGAGAGG | 1341 |
| 144702 | UCCCUUGUUUAGCUUGCUCU | 1342 |
| 144703 | CCCGCUGUCCCAUCCUGGAG | 1343 |
| 144704 | GAGCCAAUAUAAAACAGGUC | 1344 |
| 144705 | GCCUUCCUGCCUCAGGCUCU | 1345 |
| 144706 | GGCUGUAAAGCAGGCCUGAG | 1346 |
| 144707 | UAAAGCAGGCCUGAGGUCCA | 1347 |
| 144708 | GCGGAGGUGGCCGUGAAAAG | 1348 |
| 144709 | AGCCGGUAGCUGAUCCCUUG | 1349 |
| 144710 | GUGGCGGAGGUGGCCGUGAA | 1350 |
| 144711 | UACUCCACCUUCCACCCCAC | 1351 |
| 144712 | GUUCUCUAUCUAAUAUCAGU | 1352 |
| 144713 | CCCAGCUCACUGGGCCUUCU | 1353 |
| 144714 | UGGGCCAAAAGCCUUGCACU | 1354 |
| 144715 | CUCCACCUUCCACCCCACUU | 1355 |
| 144716 | GAGGUCAGUGUGGGGGCAUG | 1356 |
| 144717 | CACUGGGUAGUGGCAGAGCU | 1357 |
| 144718 | CCCAGGACUGAAAAAACUGA | 1358 |
| 144719 | CCUGCAAUUGGGUCAUGCUG | 1359 |
| 144720 | CCCCCUCCCACCCCACUUCC | 1360 |
| 144721 | GGAGAAAGGCUUGGGAUUCA | 1361 |
| 144722 | UAACCUUCACUCUGCCCCCU | 1362 |
| 144723 | CAUGGCCUUCCUGCCUCAGG | 1363 |
| 144724 | UCCAUGCCUACCCAGGACUG | 1364 |
| 144725 | CUCCACAGUGGCACCACAGA | 1365 |
| 144726 | GGCCUUCUGUGCCCGCUGUC | 1366 |
| 144727 | GUCUGGACUGCUCAGCAGGU | 1367 |
| 144728 | CUCAGCAGGUGACCUUUGCC | 1368 |
| 144729 | GCUUGCUCUGAGCUAUUAGA | 1369 |
| 144730 | UCCUUCUCUCACUAAUCCCU | 1370 |
| 144731 | UUAGAGUAGAUGUCCCGUUC | 1371 |
| 144732 | UAAAACAGGUCACAGCCCUC | 1372 |
| 144733 | AGUCCUAGCGAGGAAAAGAA | 1373 |
| 144734 | GAGGGAGAAGCUCUCACCAC | 1374 |
| 144735 | GUCUCCACCCUUGGGUUCCU | 1375 |
| 144736 | CCCAGCUACGGCAGAGGAGA | 1376 |
| 144737 | UUUAGCUUGCUCUGAGCUAU | 1377 |
| 144738 | GGACUCAUGGUCUCCACCCU | 1378 |
| 144739 | GGUCAUGCUGUCCCUUGUUU | 1379 |
| 144740 | GCCGUGAAAAGCAUGGGCAA | 1380 |
| 144741 | UUAGAAGCCUUUGGUAUCCA | 1381 |
| 144742 | GGAUCACAGGUGGAGGUCAG | 1382 |
| 144743 | CAAUUGGGUCAUGCUGUCCC | 1383 |
| 144744 | GCUCUAGGCUGUAAAGCAGG | 1384 |
| 144745 | GGUUCCUGGUGUGGGGGGGG | 1385 |
| 144746 | GUGGCAGAGCUGCCUGCAAU | 1386 |
| 144747 | GCACCACAGAUUCCCCAUUC | 1387 |
| 144748 | UUUCUAUACUCCACCUUCCA | 1388 |
| 144749 | GUGUGGGGGGGGCUAGAAGC | 1389 |
| 144750 | ACCUUCACUCUGCCCCCUCC | 1390 |
| 144751 | GCUGCAUGGCCUUCCUGCCU | 1391 |
| 144752 | UGGGGGCAUGUAACCUUCAC | 1392 |
| 144753 | GGGGGCUGGGUCUACUGUAG | 1393 |
| 144754 | GCAGAGCUGCCUGCAAUUGG | 1394 |
| 144755 | AAAAAACUGAGUCCUAGCGA | 1395 |
| 144756 | AGCUAUUAGAAGCCUUUGGU | 1396 |
| 144757 | GGGAGGAGAGGAAGGAAGGG | 1397 |
| 144758 | UCUUUUCCUGUCUCACCGAC | 1398 |
| 144759 | GAGUAGAUGUCCCGUUCUGC | 1399 |
| PL34554 | CUUACGGGCAGAGGCCAGGA | 2018 |
| PL34555 | CUCUUUCCUCAGGAGCUUCA | 2019 |
| PL34556 | AUUUAGGGGCUGGGUGACCG | 2020 |
| PL34557 | ACUGAUUUAGGGGCUGGGUG | 2021 |
| PL34558 | CUUCCCCUGACUGAUUUAGG | 2022 |
| PL34559 | GAGGCAGCUGCUCCAGGUAA | 2023 |
| PL34560 | CAUGGCACCUCUGUUCCUGC | 2024 |
| PL34561 | GCGCUCCUGGCCUCUGCCCG | 2025 |
| PL34562 | AAGCCAUCGGUCACCCAGCC | 2026 |
| n/a | ACCCUGCAUGAAGCUGAGAA | 2084 |
| n/a | GGAUUUGGACCCUGAGGUCA | 2085 |
| n/a | GUACAAGAGAUAGAAAGACC | 2086 |

### PSCK9 Spacer Sequences

TABLE 3 and TABLE 4 provide illustrative spacer sequences targeting the PCSK9 gene for use with the compositions, systems, and methods of the disclosure. In particular, TABLE 3 provides spacer sequences suitable for use in combination with an effector protein of SEQ ID NO: 32 or variants thereof (e.g., variants provided in TABLES 18 and 19). In particular, TABLE 4 provides spacer sequences suitable for use in combination with an effector protein of SEQ ID NO: 773 or variants thereof (e.g., variants provided in TABLES 16 and 17). In some embodiments, the spacer sequence comprises at least 70%, at least 80%, at least 90%, at least 91%, at least 92%, at least 93%, at least 94%, at least 95%, at least 96%, at least 97%, or at least 99%, or 100% sequence identity to a sequence as set forth in TABLE 3 or TABLE 4. In some embodiments, spacer sequences comprise at least 10, at least 11, at least 12, at least 13, at least 14, at least 15, at least 16, at least 17, at least 18, at least 19, or at least 20 contiguous nucleotides of a sequence selected from any one of SEQ ID NOs: 79-140, 208, 300-487, 799-803, 809, 822 and 1970-1995.

TABLE 3
Exemplary Spacer Sequences Targeting PCSK9
for CasPhi.12 Effector Proteins

| Target | | Spacer | |
|---|---|---|---|
| Region | Spacer | sequence (5′ to 3′), | SEQ ID |
| of PCSK9 | ID | shown as RNA | NO: |
| Exon #1 | 63561 | CGGUGGGGAGGACUGUG | 79 |
| Exon #1 | 63571 | CGUUCCGAGGAGGACGG | 80 |
| Exon #1 | 63565 | CGAGGAGGACGGCCUGG | 81 |
| Exon #1 | 63562 | GCGCAGCGGUGGAAGGU | 82 |
| Exon #2 | 63554 | AGCACCACCACGUAGGU | 83 |
| Exon #2 | 63553 | GGCCUGCAGGCGGCGGG | 84 |
| Exon #2 | 63552 | GUGAGGUAUCCCCGGCG | 85 |
| Exon #2 | 63556 | UUCCUGGCUUCCUGGUG | 86 |
| Exon #2 | 63551 | ACCAGGAAGCCAGGAAG | 87 |
| Exon #2 | 63557 | CUGGCUUCCUGGUGAAG | 88 |
| Exon #2 | 63558 | CUGGUGAAGAUGAGUGG | 89 |
| Exon #3 | 63550 | CCCCAUGUCGACUACAU | 90 |
| Exon #3 | 63545 | AUCCGCCCGGUACCGUG | 91 |
| Exon #4 | 63541 | CCGGUGGUCACUCUGUA | 92 |
| Exon #4 | 63542 | CCCGGUGGUCACUCUGU | 93 |
| Exon #5 | 63539 | GCCACGCCGGCAUCCCG | 94 |
| Exon #5 | 63537 | GCAGUUGAGCACGCGCA | 95 |
| Exon #5 | 63540 | CCUUGGCAGUUGAGCAC | 96 |
| Exon #6 | 63530 | CCGAAUAAACUCCAGGC | 97 |
| Exon #6 | 63529 | UCCGAAUAAACUCCAGG | 98 |
| Exon #6 | 63532 | AUUCGGAAAAGCCAGCU | 99 |
| Exon #6 | 63534 | UUCGGAAAAGCCAGCUG | 100 |
| Exon #6 | 63531 | GGAAAAGCCAGCUGGUC | 101 |
| Exon #6 | 63536 | UGCUGCUGCCCCUGGCG | 102 |
| Exon #6 | 63533 | AACGCCGCCUGCCAGCG | 103 |
| Exon #6 | 63527 | CCGGCAGCGGUGACCAG | 104 |
| Exon #6 | 63535 | CGGGACGAUGCCUGCCU | 105 |
| Exon #7 | 63515 | GUGGCCCCAACUGUGAU | 106 |
| Exon #7 | 63516 | GUCCCCAAAGUCCCCAG | 107 |
| Exon #7 | 63517 | GGGGACCAACUUUGGCC | 108 |
| Exon #7 | 63518 | GGGACCAACUUUGGCCG | 109 |
| Exon #7 | 63521 | GGCCGCUGUGUGGACCU | 110 |
| Exon #7 | 63520 | GCCGCUGUGUGGACCUC | 111 |
| Exon #7 | 63525 | GCCCCAGGGGAGGACAU | 112 |
| Exon #7 | 63519 | CCCCAGGGGAGGACAUC | 113 |
| Exon #7 | 63523 | GUGCCUCCAGCGACUGC | 114 |
| Exon #8 | 63513 | CAGCCAUGAUGCUGUCU | 115 |
| Exon #8 | 63511 | AGGCAGAGACUGAUCCA | 116 |
| Exon #8 | 63508 | GCAGAGAAGUGGAUCAG | 117 |
| Exon #8 | 63510 | GGCAGAGAAGUGGAUCA | 118 |
| Exon #8 | 63512 | UCUGCCAAAGAUGUCAU | 119 |
| Exon #8 | 63507 | AUGACAUCUUUGGCAGA | 120 |
| Exon #8 | 63514 | CCUGAGGACCAGCGGGU | 121 |
| Exon #8 | 63509 | GGGGUCAGUACCCGCUG | 122 |
| Exon #9 | 63506 | GCAGCUGUUUUGCAGGA | 123 |
| Exon #9 | 63500 | CCACUCCUGGAGAAACU | 124 |
| Exon #9 | 63505 | CUCCAGGAGUGGGAAGC | 125 |
| Exon #9 | 63503 | UCCAGGAGUGGGAAGCG | 126 |
| Exon #10 | 63498 | UGGGGGUGAGGGUGUCU | 127 |
| Exon #10 | 63497 | GGGGGUGAGGGUGUCUA | 128 |
| Exon #10 | 63496 | GGGGUGAGGGUGUCUAC | 129 |
| Exon #10 | 63499 | CCAGGUGCUGCCUGCUA | 130 |
| Exon #11 | 63485 | UGGGUGCCAAGGUCCUC | 131 |
| Exon #11 | 63493 | GCACCCACAAGCCGCCU | 132 |
| Exon #11 | 63484 | GGCUGACCUCGUGGCCU | 133 |
| Exon #11 | 63487 | CAUUCCAGACCUGGGGC | 134 |
| Exon #11 | 63489 | GCAUUCCAGACCUGGGG | 135 |
| Exon #11 | 63486 | ACUUUGCAUUCCAGACC | 136 |
| Exon #11 | 63490 | CAUGCUCCUUGACUUUG | 137 |
| Exon #12 | 63407 | UCGUGGCCUGUGAGGAC | 138 |
| Exon #12 | 63345 | UUCGCUGGUGCUGCCUG | 139 |
| Exon #12 | 63440 | CCAUCUGCUGCCGGAGC | 140 |
| — | n/a | GAGCAACGGCGGAAGGU | 208 |
| — | PL34711 | ACCCACCUGUGCCGCGGCGA | 799 |
| — | PL34712 | CAUGGGGCCAGGAUCCGUGG | 800 |
| — | PL34713 | UGCAGGCCUUGAAGUUGCCC | 801 |
| — | PL34714 | GUCGAGCAGGCCAGCAAGUG | 802 |
| — | PL34715 | CUCCCAGGCCUGGAGUUUAU | 803 |
| — | n/a | GAAAGACGGAGGCAGCCUGG | 809 |

TABLE 4
Exemplary Spacer Sequences Targeting PCSK9 for
CasM.265466 Effector Proteins

| Target | | | Spacer | SEQ |
|---|---|---|---|---|
| Region | | Spacer | sequence (5′ to 3′), | ID |
| of PCSK9 | PAM | ID | shown as RNA | NO: |
| Exon #4 | n/a | 129194 | GAAGCGGGUCCCGUCCUCCU | 300 |
| Exon #4 | n/a | 129195 | UCUAGGAGAUACACCUCCAC | 301 |
| Exon #4 | n/a | 129196 | ACCAUGACCCUGCCCUCGAU | 302 |
| Exon #4 | n/a | 129197 | ACCCUGCCCUCGAUUUCCCG | 303 |
| Exon #4 | n/a | 129198 | CCCUCGAUUUCCCGGUGGUC | 304 |
| Exon #4 | n/a | 129199 | UAUGCUGGUGUCUAGGAGAU | 305 |
| Exon #4 | n/a | 129200 | UGCUGGUGUCUAGGAGAUAC | 306 |
| Exon #4 | n/a | 129201 | GUCACUCUGUAUGCUGGUGU | 307 |
| Exon #4 | n/a | 129202 | UGGAAGCGGGUCCCGUCCUC | 308 |
| Exon #4 | n/a | 129203 | CUGGUGUCUAGGAGAUACAC | 309 |
| Exon #4 | n/a | 129204 | GGAGAUACACCUCCACCAGG | 310 |
| Exon #4 | n/a | 129205 | GUGUCUAGGAGAUACACCUC | 311 |
| Exon #4 | n/a | 129206 | CACCUCCACCAGGCUGCCUC | 312 |
| Exon #4 | n/a | 129207 | GACACCAGCAUACAGAGUGA | 313 |
| Exon #4 | n/a | 129209 | UGCCCGAGGAGGACGGGACC | 314 |
| Exon #4 | n/a | 129210 | GUGGAGGUGUAUCUCCUAGA | 315 |
| Exon #4 | n/a | 129211 | UCUCCUAGACACCAGCAUAC | 316 |
| Exon #4 | n/a | 129213 | CAGAGUGACCACCGGGAAAU | 317 |
| Exon #4 | n/a | 129214 | UAUCUCCUAGACACCAGCAU | 318 |
| Exon #4 | n/a | 129215 | ACCACCGGGAAAUCGAGGGC | 319 |
| Exon #4 | n/a | 129216 | GAGGUGUAUCUCCUAGACAC | 320 |
| Exon #4 | n/a | 129217 | CCCGAGGAGGACGGGACCCG | 321 |
| Exon #5 | n/a | 129171 | CCCUUCCCUUGGCAGUUGAG | 322 |
| Exon #5 | n/a | 129172 | GCAGUUGAGCACGCGCAGGC | 323 |
| Exon #5 | n/a | 129174 | ACCGUGCCCUUCCCUUGGCA | 324 |
| Exon #5 | n/a | 129178 | GCCACGCCGGCAUCCCGGCC | 325 |
| Exon #5 | n/a | 129179 | ACCACCCCUGCCAGGUGGGU | 326 |
| Exon #5 | n/a | 129182 | GCAGGGGUGGUCAGCGGCCG | 327 |
| Exon #5 | n/a | 129183 | CUCAACUGCCAAGGGAAGGG | 328 |
| Exon #5 | n/a | 129189 | GUCAGCGGCCGGGAUGCCGG | 329 |
| Exon #5 | n/a | 129192 | CGCGUGCUCAACUGCCAAGG | 330 |
| Exon #5 | n/a | 129193 | GCACCCACCUGGCAGGGGUG | 331 |
| Exon #6 | n/a | 129138 | CCGGCAGCGGUGACCAGCAC | 332 |
| Exon #6 | n/a | 129139 | GCUUUUCCGAAUAAACUCCA | 333 |
| Exon #6 | n/a | 129140 | UACCCACCCGCCAGGGGCAG | 334 |
| Exon #6 | n/a | 129141 | GACCAGCUGGCUUUUCCGAA | 335 |
| Exon #6 | n/a | 129142 | CCCACCCGCCAGGGGCAGCA | 336 |
| Exon #6 | n/a | 129143 | GGGAGUAGAGGCAGGCAUCG | 337 |
| Exon #6 | n/a | 129145 | ACCAGCACGACCCCAGCCCU | 338 |
| Exon #6 | n/a | 129146 | GAGGCAGGCAUCGUCCCGGA | 339 |
| Exon #6 | n/a | 129150 | CCCCUGGCGGGUGGGUACAG | 340 |
| Exon #6 | n/a | 129151 | GGGUCGUGCUGGUCACCGCU | 341 |
| Exon #6 | n/a | 129152 | CUGGUCACCGCUGCCGGCAA | 342 |
| Exon #6 | n/a | 129153 | GUCACCGCUGCCGGCAACUU | 343 |
| Exon #6 | n/a | 129154 | CUGCCCCUGGCGGGUGGGUA | 344 |
| Exon #6 | n/a | 129156 | GAGUUUAUUCGGAAAAGCCA | 345 |
| Exon #6 | n/a | 129157 | GCGAGGGCUGGGGUCGUGCU | 346 |
| Exon #6 | n/a | 129158 | CUGCUGCCCCUGGCGGGUGG | 347 |
| Exon #6 | n/a | 129160 | UUCGGAAAAGCCAGCUGGUC | 348 |
| Exon #6 | n/a | 129163 | CCUGCCUCUACUCCCCAGCC | 349 |
| Exon #6 | n/a | 129165 | CCAGCGCCUGGCGAGGGCUG | 350 |
| Exon #6 | n/a | 129167 | CCGGCAACUUCCGGGACGAU | 351 |
| Exon #7 | n/a | 129107 | UCCUCCCCUGGGGCAAAGAG | 352 |
| Exon #7 | n/a | 129108 | GAGGCACCAAUGAUGUCCUC | 353 |
| Exon #7 | n/a | 129109 | GGCAUUGGUGGCCCCAACUG | 354 |
| Exon #7 | n/a | 129110 | AUGUCCUCCCCUGGGGCAAA | 355 |
| Exon #7 | n/a | 129111 | ACACAAAGCAGGUGCUGCAG | 356 |
| Exon #7 | n/a | 129112 | GUGGCCCCAACUGUGAUGAC | 357 |
| Exon #7 | n/a | 129113 | CUGCAGUCGCUGGAGGCACC | 358 |
| Exon #7 | n/a | 129115 | CAGUCGCUGGAGGCACCAAU | 359 |
| Exon #7 | n/a | 129118 | GUCCCCAAAGUCCCCAGGGU | 360 |
| Exon #7 | n/a | 129120 | GGGCAAAGAGGUCCACACAG | 361 |
| Exon #7 | n/a | 129122 | GUGCCUCCAGCGACUGCAGC | 362 |
| Exon #7 | n/a | 129124 | CCUCCAGCGACUGCAGCACC | 363 |
| Exon #7 | n/a | 129125 | GCCGCUGUGUGGACCUCUUU | 364 |
| Exon #7 | n/a | 129127 | GACCUCUUUGCCCCAGGGGA | 365 |
| Exon #7 | n/a | 129129 | ACCCUGGGGACUUUGGGGAC | 366 |
| Exon #7 | n/a | 129131 | UGGACCUCUUUGCCCCAGGG | 367 |
| Exon #7 | n/a | 129132 | CAGCACCUGCUUUGUGUCAC | 368 |
| Exon #7 | n/a | 129134 | GGGACCAACUUUGGCCGCUG | 369 |
| Exon #7 | n/a | 129135 | GGGACUUUGGGGACCAACUU | 370 |
| Exon #7 | n/a | 129136 | UGUGGACCUCUUUGCCCCAG | 371 |
| Exon #7 | n/a | 129137 | CCCCAGGGGAGGACAUCAUU | 372 |
| Exon #8 | n/a | 129076 | GAUCAGUCUCUGCCUCAACU | 373 |
| Exon #8 | n/a | 129080 | GCAGAGAAGUGGAUCAGUCU | 374 |
| Exon #8 | n/a | 129081 | AGCUCCGGCUCGGCAGACAG | 375 |
| Exon #8 | n/a | 129082 | GUCCUCAGGGAACCAGGCCU | 376 |
| Exon #8 | n/a | 129083 | AUGACAUCUUUGGCAGAGAA | 377 |
| Exon #8 | n/a | 129084 | ACAUCUUUGGCAGAGAAGUG | 378 |
| Exon #8 | n/a | 129089 | CUGUCUGCCGAGCCGGAGCU | 379 |
| Exon #8 | n/a | 129091 | CAGCCAUGAUGCUGUCUGCC | 380 |
| Exon #8 | n/a | 129092 | AUCCACUUCUCUGCCAAAGA | 381 |
| Exon #8 | n/a | 129094 | AGGCCUGGUUCCCUGAGGAC | 382 |
| Exon #8 | n/a | 129096 | AUGCUGUCUGCCGAGCCGGA | 383 |
| Exon #8 | n/a | 129099 | AGGCAGAGACUGAUCCACUU | 384 |
| Exon #8 | n/a | 129101 | CCAAAGAUGUCAUCAAUGAG | 385 |
| Exon #8 | n/a | 129102 | UCUGCCGAGCCGGAGCUCAC | 386 |
| Exon #8 | n/a | 129103 | GCCGAGUUGAGGCAGAGACU | 387 |
| Exon #8 | n/a | 129104 | UCAUCAAUGAGGCCUGGUUC | 388 |
| Exon #9 | n/a | 129051 | UGGCCAUCCGUGUAGGCCCC | 389 |
| Exon #9 | n/a | 129053 | GAGCAGCUCAGCAGCUCCUC | 390 |
| Exon #9 | n/a | 129054 | CGCUCGCCCCGCCGCUUCCC | 391 |
| Exon #9 | n/a | 129056 | GAGAAACUGGAGCAGCUCAG | 392 |
| Exon #9 | n/a | 129057 | GCCAUCCGUGUAGGCCCCGA | 393 |
| Exon #9 | n/a | 129058 | UAGGCCCCGAGUGUGCUGAC | 394 |
| Exon #9 | n/a | 129059 | GGCCCCGAGUGUGCUGACCA | 395 |
| Exon #9 | n/a | 129061 | GGAAGCGGCGGGGCGAGCGC | 396 |
| Exon #9 | n/a | 129062 | CUGAGCUGCUCCAGUUUCUC | 397 |
| Exon #9 | n/a | 129065 | UGGUCAGCACACUCGGGGCC | 398 |
| Exon #9 | n/a | 129068 | CUCCAGUUUCUCCAGGAGUG | 399 |
| Exon #9 | n/a | 129070 | GCAGCUGUUUUGCAGGACUG | 400 |
| Exon #9 | n/a | 129071 | AGGAGCUGCUGAGCUGCUCC | 401 |
| Exon #9 | n/a | 129074 | AGCUGCUCCAGUUUCUCCAG | 402 |
| Exon #9 | n/a | 129075 | GUCAGCACACUCGGGGCCUA | 403 |
| Exon #10 | n/a | 129012 | GAGCUGUGUGGACGCUGCAG | 404 |
| Exon #10 | n/a | 129013 | GCAGUGGACACGGGUCCCCA | 405 |
| Exon #10 | n/a | 129014 | GCGUAGACACCCUCACCCCC | 406 |
| Exon #10 | n/a | 129018 | GACACCCUCACCCCCAAAAG | 407 |
| Exon #10 | n/a | 129022 | GACACGGGUCCCCAUGCUGG | 408 |
| Exon #10 | n/a | 129023 | GGGUAGCAGGCAGCACCUGG | 409 |
| Exon #10 | n/a | 129024 | GCAGGCAGCACCUGGCAAUG | 410 |
| Exon #10 | n/a | 129025 | GUGGAGCUGUGUGGACGCUG | 411 |
| Exon #10 | n/a | 129026 | GCAAUGGCGUAGACACCCUC | 412 |
| Exon #10 | n/a | 129034 | CCAGGUGCUGCCUGCUACCC | 413 |
| Exon #10 | n/a | 129038 | GGGGUGAGGGUGUCUACGCC | 414 |
| Exon #10 | n/a | 129041 | CGCCAUUGCCAGGUGCUGCC | 415 |
| Exon #10 | n/a | 129043 | AGGGUGUCUACGCCAUUGCC | 416 |
| Exon #10 | n/a | 129044 | UCUACGCCAUUGCCAGGUGC | 417 |
| Exon #10 | n/a | 129046 | CCGGGCCCACAACGCUUUUG | 418 |
| Exon #10 | n/a | 129047 | CAGCGUCCACACAGCUCCAC | 419 |
| Exon #10 | n/a | 129048 | GGGACCCGUGUCCACUGCCA | 420 |
| Exon #11 | n/a | 128978 | UGGGUGCCAAGGUCCUCCAC | 421 |
| Exon #11 | n/a | 128979 | CCAAGGUCCUCCACCUCCCA | 422 |
| Exon #11 | n/a | 128981 | ACUUUGCAUUCCAGACCUGG | 423 |
| Exon #11 | n/a | 128982 | GCAGCAGGAAGCGUGGAUGC | 424 |
| Exon #11 | n/a | 128986 | GGCUGACCUCGUGGCCUCAG | 425 |
| Exon #11 | n/a | 128987 | GCCUCAGCACAGGCGGCUUG | 426 |
| Exon #11 | n/a | 128989 | ACCUCGUGGCCUCAGCACAG | 427 |
| Exon #11 | n/a | 128990 | CUCCUUGACUUUGCAUUCCA | 428 |
| Exon #11 | n/a | 128991 | CAUUCCAGACCUGGGGCAUG | 429 |
| Exon #11 | n/a | 128992 | GGUGCCAAGGUCCUCCACCU | 430 |
| Exon #11 | n/a | 128993 | GUUGGGCUGACCUCGUGGCC | 431 |
| Exon #11 | n/a | 128994 | GGGCAUGGCAGCAGGAAGCG | 432 |
| Exon #11 | n/a | 128995 | AGGGGCCGGGAUUCCAUGCU | 433 |
| Exon #11 | n/a | 128996 | UGCUGAGGCCACGAGGUCAG | 434 |
| Exon #11 | n/a | 128998 | GCACCCACAAGCCGCCUGUG | 435 |
| Exon #11 | n/a | 128999 | CCCCAGGUCUGGAAUGCAAA | 436 |
| Exon #11 | n/a | 129002 | GAGGACCUUGGCACCCACAA | 437 |
| Exon #11 | n/a | 129003 | CUGAGGCCACGAGGUCAGCC | 438 |
| Exon #11 | n/a | 129004 | GAAUGCAAAGUCAAGGAGCA | 439 |
| Exon #11 | n/a | 129005 | CCAUGCCCCAGGUCUGGAAU | 440 |
| Exon #11 | n/a | 129006 | CUGCCAUGCCCCAGGUCUGG | 441 |
| Exon #11 | n/a | 129007 | GGAGGUGGAGGACCUUGGCA | 442 |
| Exon #11 | n/a | 129008 | AGGCCACGAGGUCAGCCCAA | 443 |
| Exon #11 | n/a | 129009 | CAAAGUCAAGGAGCAUGGAA | 444 |
| Exon #11 | n/a | 129010 | CAGCUCCCACUGGGAGGUGG | 445 |
| Exon #11 | n/a | 129011 | GAAUCCCGGCCCCUCAGGAG | 446 |
| Exon #12 | n/a | 128864 | GCUGUAAAAAGGCAACAGAG | 447 |
| Exon #12 | n/a | 128865 | CAAAAGCAAAACAGGUCUAG | 448 |
| Exon #12 | n/a | 128867 | AAUGUCUGCUUGCUUGGGUG | 449 |
| Exon #12 | n/a | 128868 | AAAAUGCUACAAAACCCAGA | 450 |
| Exon #12 | n/a | 128870 | CUUGCUUGGGUGGGGCUGGU | 451 |
| Exon #12 | n/a | 128871 | CUACAAAACCCAGAAUAAAU | 452 |
| Exon #12 | n/a | 128876 | UCUGCUUGCUUGGGUGGGGC | 453 |
| Exon #12 | n/a | 128878 | GGUGGGGCUGGUGCUCAAGG | 454 |
| Exon #12 | n/a | 128879 | AAAAGGCAACAGAGAGGACA | 455 |
| Exon #12 | n/a | 128881 | AUAAAAAUGCUACAAAACCC | 456 |
| Exon #12 | n/a | 128882 | GUCUGUGUUCCCCUUCCCAG | 457 |
| Exon #12 | n/a | 128883 | UUCCCCUUCCCAGCCUCACU | 458 |
| Exon #12 | n/a | 128884 | UAAAAAGGCAACAGAGAGGA | 459 |
| Exon #12 | n/a | 128885 | UCUUCAAGUUACAAAAGCAA | 460 |
| Exon #12 | n/a | 128887 | GUGCUCAAGGAGGGACAGUU | 461 |
| Exon #12 | n/a | 128889 | GGGCUGGUGCUCAAGGAGGG | 462 |
| Exon #12 | n/a | 128891 | CUUGGGUGGGGCUGGUGCUC | 463 |
| Exon #12 | n/a | 128892 | CAAAACCCAGAAUAAAUAUC | 464 |
| Exon #12 | n/a | 128893 | UGUUCCCCUUCCCAGCCUCA | 465 |
| Exon #12 | n/a | 128894 | GACCUGUUUUGCUUUUGUAA | 466 |
| Exon #12 | n/a | 128896 | CUUUUGUAACUUGAAGAUAU | 467 |
| Exon #12 | n/a | 128897 | UCCUCUCUGUUGCCUUUUUA | 468 |
| Exon #12 | n/a | 128898 | GGUCUGUCCUCUCUGUUGCC | 469 |
| Exon #12 | n/a | 128904 | UUCUGGGUUUUGUAGCAUUU | 470 |
| Exon #12 | n/a | 128908 | UCCCUCCUUGAGCACCAGCC | 471 |
| Exon #12 | n/a | 128909 | AAGAUAUUUAUUCUGGGUUU | 472 |
| Exon #12 | n/a | 128914 | UUUAUUCUGGGUUUUGUAGC | 473 |
| Exon #12 | n/a | 128916 | ACUUGAAGAUAUUUAUUCUG | 474 |
| Exon #12 | n/a | 128917 | AGGCUGGGAAGGGGAACACA | 475 |
| Exon #12 | n/a | 128920 | UCUUUUGGGUCUGUCCUCUC | 476 |
| Exon #12 | n/a | 128921 | UUUUGCUUUUGUAACUUGAA | 477 |
| Exon #12 | n/a | 128923 | GGUUUUGUAGCAUUUUUAUU | 478 |
| Exon #12 | n/a | 128925 | AGCACCAGCCCCACCCAAGC | 479 |
| Exon #12 | n/a | 128929 | GGAAGGGGAACACAGACCAG | 480 |
| Exon #12 | n/a | 128930 | CCGGCUCCGGCAGCAGAUGG | 481 |
| Exon #12 | n/a | 128933 | GGAGGUCCCAGGGAGGGCAC | 482 |
| Exon #12 | n/a | 128950 | GGAUGGGGCUGUCACUGGAG | 483 |
| Exon #12 | n/a | 128960 | CAGUGCCCUCCCUGGGACCU | 484 |
| Exon #12 | n/a | 128964 | CCAUCUGCUGCCGGAGCCGG | 485 |
| Exon #12 | n/a | 128969 | ACAGCCCCAUCCCAGGAUGG | 486 |
| Exon #12 | n/a | 128977 | CUGCCGGAGCCGGCACCUGG | 487 |
| — | n/a | n/a | UAGAACCUUGAUGACAUAGC | 822 |
| — | TCTA | PL34563 | CACCCGCACCUUGGCGCAGC | 1970 |
| — | TTTA | PL34564 | GGGCCAGGAUCCGUGGAGGU | 1971 |
| — | TATA | PL34565 | GCUCACCAGCUCCAGCAGGU | 1972 |
| — | ATTA | PL34566 | GCUUCUGCAGGCCUUGAAGU | 1973 |
| — | TTTA | PL34567 | GGGGUCUUACCGGGGGGCUG | 1974 |
| — | AGTG | PL34568 | GAAAGACGGAGGCAGCCUGG | 1975 |
| — | TTTA | PL34569 | CUUACCUGUCUGUGGAAGCG | 1976 |
| — | TATA | PL34570 | UUCGUCGAGCAGGCCAGCAA | 1977 |
| — | TGTA | PL34571 | GGGCCAUCACUUACCUAUGA | 1978 |
| — | TTTA | PL34572 | UUCCUCCCAGGCCUGGAGUU | 1979 |
| — | GGTA | PL34573 | AUGACCUGGAAAGGUGAGGA | 1980 |
| — | TCTA | PL34574 | CACCAGGCAUUGCAGCCAUG | 1981 |
| — | ATTA | PL34575 | CUUACCUGCCCCAUGGGUGC | 1982 |
| — | AATA | PL34576 | CAGUCACCUCCAUGCGCUCG | 1983 |
| — | CTTG | PL34577 | ACUCUAAGGCCCAAGGGGGC | 1984 |
| — | AATA | PL34578 | CCCCAGGCUGCAGCUCCCAC | 1985 |
| — | GGTA | PL34579 | GCAGGUGACCGUGGCCUGCG | 1986 |
| — | AATG | PL34580 | CCUCGCCGCGGCACAGGUGG | 1987 |
| — | GTTG | PL34581 | CCAGGCAACCUCCACGGAUC | 1988 |
| — | TATG | PL34582 | GCGACCUGCUGGAGCUGGUG | 1989 |
| — | TCTA | PL34583 | AGUGGCGACCUGCUGGAGCU | 1990 |
| — | ACTG | PL34584 | ACUGUCACACUUGCUGGCCU | 1991 |
| — | AGTG | PL34585 | CUCCCCAGCCUCAGCUCCCG | 1992 |
| — | CCTG | PL34586 | GCCCCAACUGUGAUGACCUG | 1993 |
| — | ACTG | PL34587 | CCCCCCAGCACCCAUGGGGC | 1994 |
| — | CCTG | PL34588 | CAAAACAGCUGCCAACCUGC | 1995 |

### ANGPTL3 Spacer Sequences

TABLES 5 and 6 provides illustrative spacer sequences targeting the ANGPTL3 gene for use with the compositions, systems, and methods of the disclosure. In particular, TABLE 5 provides spacer sequences suitable for use in combination with an effector protein of SEQ ID NO: 32 or variants thereof (e.g., variants provided in TABLES 18 and 19). In particular, TABLE 6 provides spacer sequences suitable for use in combination with an effector protein of SEQ ID NO: 773 or variants thereof (e.g., variants provided in TABLES 16 and 17). In some embodiments, the spacer sequence comprises at least 70%, at least 80%, at least 90%, at least 91%, at least 92%, at least 93%, at least 94%, at least 95%, at least 96%, at least 97%, or at least 99%, or 100% sequence identity to a sequence as set forth in TABLES 5 and 6. In some embodiments, spacer sequences comprise at least 10, at least 11, at least 12, at least 13, at least 14, at least 15, at least 16, at least 17, at least 18, at least 19, or at least 20 contiguous nucleotides of a sequence selected from any one of SEQ ID NOs: 806-808 and 1996-2017.

TABLE 5
Exemplary Spacer Sequences Targeting ANGPTL3
for CasPhi.12 Effector Proteins

| | Spacer sequence (5′ to 3′), | |
|---|---|---|
| Spacer ID | shown as RNA | SEQ ID NO: |
| PL34718 | UACUUACUUUAAGUGAAGUU | 806 |
| PL34719 | UAUCAGCUCAGAAGGACUAG | 807 |
| PL34720 | AUUCUAGGCAUUCCUGCUGA | 808 |

TABLE 6
Exemplary Spacer Sequences Targeting ANGPTL3
for CasM.265466 Effector Proteins

| | | Spacer sequence | |
|---|---|---|---|
| Spacer | | (5′ to 3′), shown as | SEQ ID |
| ID | PAM | RNA | NO: |
| PL34532 | GTTG | CUUACUUUAAGUGAAGUUAC | 1996 |
| PL34533 | CCTA | UUUUCUACUUACUUUAAGUG | 1997 |
| PL34534 | GCTG | UCCAGACUUUUGUAGAAAAA | 1998 |
| PL34535 | CCTG | AAAUACUGACUUACCUGAUU | 1999 |
| PL34536 | ACTG | UCAGCUCAGAAGGACUAGUA | 2000 |
| PL34537 | CCTA | UCUUACCAUCAUGUUUUACA | 2001 |
| PL34538 | CATG | UUGAUUCUAGGCAUUCCUGC | 2002 |
| PL34539 | GGTG | UUCAGGUAGUCCAUGGACAU | 2003 |
| PL34540 | TCTG | GUCCCCUUACCAUCAAGCCU | 2004 |
| PL34541 | GATG | AAACUUUUCUUUUCAGGAGA | 2005 |
| PL34542 | CTTG | UCAGAAAAAGAUACCUGAAU | 2006 |
| PL34543 | CGTG | UCUCCUUUAGGAGGCUGGUG | 2007 |
| PL34544 | TGTG | UCUUGUUUUUCUACAAAAGU | 2008 |
| PL34545 | TCTG | AAAGAAAUAGAAAAUCAGGU | 2009 |
| PL34546 | TTTG | AAUACUAGUCCUUCUGAGCU | 2010 |
| PL34547 | TGTG | AGAAAUGUAAAACAUGAUGG | 2011 |
| PL34548 | CCTG | CAUUCAGCAGGAAUGCCUAG | 2012 |
| PL34549 | CCTG | GUGGUACAUUCAGCAGGAAU | 2013 |
| PL34550 | GGTA | AAUUAAUGUCCAUGGACUAC | 2014 |
| PL34551 | TTTG | GUUUUGGGAGGCUUGAUGGU | 2015 |
| PL34552 | TCTG | GGCCCAACCAAAAUUCUCCU | 2016 |
| PL34553 | TCTG | UCCAGAGGGUUAUUCAGGUA | 2017 |

In some embodiments, the spacer sequence comprises one or more nucleobase alterations at one or more positions in any one of the sequences of TABLES 1-13. Alternative nucleobases can be any one or more of A, C, G, T or U, or a deletion, or an insertion. In some embodiments, the U is pseudouracil. By way of non-limiting example, a guanine nucleobase could be replaced with the nucleobase of any one of a cytosine, adenosine, thymine, and uracil. In some instance, the spacer sequence comprises only one nucleobase alterations relative to a sequence of TABLES 1-13. In some instance, the spacer sequence comprises not more than 1, not more than 2, nor more than 3, or not more than 4 nucleobase alterations relative to a sequence of TABLES 1-13.

Targeting locations listed for any of the spacer sequences provided in TABLES 1-6 or the exemplary guide sequences in TABLES 8-13 should not be construed as limiting targeting locations. For example, a spacer sequence that is listed as targeting exon 1 category should not be construed as limited to a target sequence only in exon 1 and no other location in the APOC3, PCSK9, or ANGPLT3 gene.

### Repeat Sequences

Guide nucleic acids described herein may comprise one or more repeat sequences. In some embodiments, a repeat sequence comprises a nucleotide sequence that is not complementary to a target sequence of a target nucleic acid. In some embodiments, a repeat sequence comprises a nucleotide sequence that may interact with an effector protein. In some embodiments, a repeat sequence includes a nucleotide sequence that is capable of forming a guide nucleic acid-effector protein complex (e.g., a RNP complex). In some embodiments, the repeat sequence may also be referred to as a “protein-binding segment.”

In some embodiments, the repeat sequence is between 10 and 50, 12 and 48, 14 and 46, 16 and 44, and 18 and 42 nucleotides in length.

In some embodiments, a repeat sequence is adjacent to a spacer sequence. In some embodiments, a repeat sequence is followed by a spacer sequence in the 5′ to 3′ direction. In some embodiments, a guide nucleic acid comprises a repeat sequence linked to a spacer sequence, which may be a direct link or by any suitable linker, examples of which are described herein.

In some embodiments, the repeat sequence is adjacent to an intermediary RNA sequence. In some embodiments, a repeat sequence is 3′ to an intermediary RNA sequence. In some embodiments, an intermediary RNA sequence is followed by a repeat sequence, which is followed by a spacer sequence in the 5′ to 3′ direction. In some embodiments, a repeat sequence is linked to a spacer sequence and/or an intermediary RNA sequence.

In some embodiments, a guide nucleic acid comprises a repeat sequence that is at least 80%, at least 90%, at least 92%, at least 95%, at least 97%, or at least 99%, or 100% identical to a sequence that is provided in TABLE 7. In some embodiments, guide nucleic acids comprise a repeat sequence, wherein the repeat sequence comprises at least 10, at least 12, at least 14, at least 16, at least 18 or at least 20 contiguous nucleotides of a sequence provided in TABLE 7.

TABLE 7
Exemplary Repeat Sequences

| Repeat sequence | | SEQ |
|---|---|---|
| (shown as RNA), 5′- 3′ | Cas protein | ID NO: |
| GUAGAUUGCUCCUUACGAGGAGAC | CasPhi.12 | 16 |
| CUUUCAAGACUAAUAGAUUGCUCC | CasPhi.12 | 38 |
| UUACGAGGAGAC | | |
| AUAGAUUGCUCCUUACGAGGAGAC | CasPhi.12 | 39 |
| UAGAUUGCUCCUUACGAGGAGAC | CasPhi.12 | 40 |
| AGAUUGCUCCUUACGAGGAGAC | CasPhi.12 | 41 |
| GAUUGCUCCUUACGAGGAGAC | CasPhi.12 | 42 |
| AUUGCUCCUUACGAGGAGAC | CasPhi.12 | 43 |
| AAGGAUGCCAAAC | CasM.265466 | 488 |

In some embodiments, guide nucleic acids comprise more than one repeat sequence (e.g., two or more, three or more, or four or more repeat sequences). In some embodiments, a guide nucleic acid comprises more than one repeat sequence separated by another sequence of the guide nucleic acid. For example, in some embodiments, a guide nucleic acid comprises two repeat sequences, wherein the first repeat sequence is followed by a spacer sequence, and the spacer sequence is followed by a second repeat sequence in the 5′ to 3′ direction. In some embodiments, the more than one repeat sequences are identical. In some embodiments, the more than one repeat sequences are not identical.

In some embodiments, the repeat sequence comprises two sequences that are complementary to each other and hybridize to form a double stranded RNA duplex (dsRNA duplex). In some embodiments, the two sequences are not directly linked and hybridize to form a stem loop structure. In some embodiments, the dsRNA duplex comprises 5, 10, 15, 20 or 25 base pairs (bp). In some embodiments, not all nucleotides of the dsRNA duplex are paired, and therefore the duplex forming sequence may include a bulge. In some embodiments, the repeat sequence comprises a hairpin or stem-loop structure, optionally at the 5′ portion of the repeat sequence. In some embodiments, a strand of the stem portion comprises a sequence and the other strand of the stem portion comprises a sequence that is at least partially, complementary. In some embodiments, such sequences may have 65% to 100% complementarity (e.g., at least 65%, at least 70%, at least 75%, at least 80%, at least 85%, at least 90%, at least 95%, or 100% complementarity). In some embodiments, a guide nucleic acid comprises nucleotide sequence that when involved in hybridization events may hybridize over one or more segments such that intervening or adjacent segments are not involved in the hybridization event (e.g., a bulge, a loop structure or hairpin structure, etc.).

In some embodiments, guide nucleic acids comprise a spacer sequence that is at least 80%, at least 85%, at least 90%, at least 95%, or 100% identical to any one of the sequences as set forth in TABLES 1, 3, and 5; and a repeat sequence that is at least 80%, at least 85%, at least 90%, at least 95%, or 100% identical to any one of SEQ ID NOs: 16 or 38-43.

In some embodiments, guide nucleic acids comprise a spacer sequence that is at least 80%, at least 85%, at least 90%, at least 95%, or 100% identical to any one of the sequences as set forth in TABLES 2, 4, and 6; and a repeat sequence that is at least 80%, at least 85%, at least 90%, at least 95%, or 100% identical to SEQ ID NO: 488.

### Intermediary Sequences

Guide nucleic acids described herein may comprise one or more intermediary sequences. In general, an intermediary sequence used in the present disclosure is not transactivated or transactivating. An intermediary sequence may also be referred to as an intermediary RNA, although it may comprise deoxyribonucleotides instead of or in addition to ribonucleotides, and/or modified bases. In general, the intermediary sequence non-covalently binds to an effector protein. In some embodiments, the intermediary sequence forms a secondary structure, for example in a cell, and an effector protein binds the secondary structure.

In some embodiments, a length of the intermediary sequence is at least 30, 50, 70, 90, 110, 130, 150, 170, 190, or 210 linked nucleotides. In some embodiments, a length of the intermediary sequence is not greater than 30, 50, 70, 90, 110, 130, 150, 170, 190, or 210 linked nucleotides. In some embodiments, the length of the intermediary sequence is about 30 to about 210, about 60 to about 210, about 90 to about 210, about 120 to about 210, about 150 to about 210, about 180 to about 210, about 30 to about 180, about 60 to about 180, about 90 to about 180, about 120 to about 180, or about 150 to about 180 linked nucleotides.

An intermediary sequence may also comprise or form a secondary structure (e.g., one or more hairpin loops) that facilitates the binding of an effector protein to a guide nucleic acid and/or modification activity of an effector protein on a target nucleic acid (e.g., a hairpin region). An intermediary sequence may comprise from 5′ to 3′, a 5′ region, a hairpin region, and a 3′ region. In some embodiments, the 5′ region may hybridize to the 3′ region. In some embodiments, the 5′ region of the intermediary sequence does not hybridize to the 3′ region.

In some embodiments, the hairpin region may comprise a first sequence, a second sequence that is reverse complementary to the first sequence, and a stem-loop linking the first sequence and the second sequence. In some embodiments, an intermediary sequence comprises a stem-loop structure comprising a stem region and a loop region. In some embodiments, the stem region is 4 to 8 linked nucleotides in length. In some embodiments, the stem region is 5 to 6 linked nucleotides in length. In some embodiments, the stem region is 4 to 5 linked nucleotides in length. In some embodiments, an intermediary sequence comprises a pseudoknot (e.g., a secondary structure comprising a stem at least partially hybridized to a second stem or half-stem secondary structure). An effector protein may interact with an intermediary sequence comprising a single stem region or multiple stem regions. In some embodiments, the nucleotide sequences of the multiple stem regions are identical to one another. In some embodiments, the nucleotide sequences of at least one of the multiple stem regions is not identical to those of the others. In some embodiments, an intermediary sequence comprises 1, 2, 3, 4, 5 or more stem regions.

In some embodiments, an intermediary sequence comprises a nucleotide sequence that is at least 65%, at least 70%, at least 75%, at least 80%, at least 85%, at least 90%, at least 95%, at least 97%, or at least 98%, at least 99%, or 100% identical to the sequence: ACAGCUUAUUUGGAAGCUGAAAUGUGAGGUUUAUAACACUCACAAGAAUCCU (SEQ ID NO: 489). In some embodiments, an intermediary sequence comprises at least 5, at least 6, at least 7, at least 8, at least 9, at least 10, at least 20, at least 30, at least 40, at least 45, or at least 50 contiguous nucleotides of any one of SEQ ID NO: 489. Such an intermediary sequence may be useful in a guide nucleic acid that is to be used with an effector protein that is at least 80%, at least 85%, at least 90%, at least 95%, at least 98%, at least 99% or 100% identical to any of SEQ ID NOs: 773-793.

### Handle Sequence

In some embodiments, compositions, systems and methods described herein comprise the nucleic acid, wherein the nucleic acid comprises a handle sequence. In some embodiments, the handle sequence comprises an intermediary sequence. In some embodiments, the intermediary sequence is at the 3′-end of the handle sequence. In some embodiments, the intermediary sequence is at the 5′-end of the handle sequence. In some embodiments, the handle sequence further comprises one or more of linkers and repeat sequences. In some embodiments, the linker comprises a sequence of 5′-GAAA-3′ (SEQ ID NO: 44). In some embodiments, the intermediary sequence is 5′ to the repeat sequence. In some embodiments, the intermediary sequence is 5′ to the linker. In some embodiments, the intermediary sequence is 3′ to the repeat sequence. In some embodiments, the intermediary sequence is 3′ to the linker. In some embodiments, the repeat sequence is 3′ to the linker. In some embodiments, the repeat sequence is 5′ to the linker.

In some embodiments, an sgRNA may include a handle sequence having a hairpin region, as well as a linker and a repeat sequence. The sgRNA having a handle sequence can have a hairpin region positioned 3′ of the linker and/or repeat sequence. The sgRNA having a handle sequence can have a hairpin region positioned 5′ of the linker and/or repeat sequence. The hairpin region may include a first sequence, a second sequence that is reverse complementary to the first sequence, and a stem-loop linking the first sequence and the second sequence.

In some embodiments, an effector protein may recognize a secondary structure of a handle sequence. In some embodiments, at least a portion of the handle sequence interacts with an effector protein described herein. Accordingly, in some embodiments, at least a portion of the intermediary sequence interacts with the effector protein described herein. In some embodiments, both, at least a portion of the intermediary sequence and at least a portion of the repeat sequence, interacts with the effector protein. In general, the handle sequence is capable of interacting (e.g., non-covalent binding) with any one of the effector proteins described herein.

In some embodiments, the handle sequence of an sgRNA comprises a stem-loop structure comprising a stem region and a loop region. In some embodiments, the stem region is 4 to 8 linked nucleotides in length. In some embodiments, the stem region is 5 to 6 linked nucleotides in length. In some embodiments, the stem region is 4 to 5 linked nucleotides in length. In some embodiments, the sgRNA comprises a pseudoknot (e.g., a secondary structure comprising a stem at least partially hybridized to a second stem or half-stem secondary structure). An effector protein may recognize an sgRNA comprising multiple stem regions. In some embodiments, the nucleotide sequences of the multiple stem regions are identical to one another. In some embodiments, the nucleotide sequences of at least one of the multiple stem regions is not identical to those of the others. In some embodiments, the sgRNA comprises at least 2, at least 3, at least 4, or at least 5 stem regions.

A handle sequence may include deoxyribonucleosides, ribonucleosides, chemically modified nucleosides, or any combination thereof. In some embodiments, a length of the handle sequence is at least 30, 50, 70, 90, 110, 130, 150, 170, 190, or 210 linked nucleotides. In some embodiments, a length of the handle sequence is not greater than 30, 50, 70, 90, 110, 130, 150, 170, 190, or 210 linked nucleotides. In some embodiments, the length of the handle sequence is about 30 to about 210, about 60 to about 210, about 90 to about 210, about 120 to about 210, about 150 to about 210, about 180 to about 210, about 30 to about 180, about 60 to about 180, about 90 to about 180, about 120 to about 180, or about 150 to about 180 linked nucleotides.

In some embodiments, the length of a handle sequence in an sgRNA is not greater than 50, 56, 66, 67, 68, 69, 70, 71, 72, 73, 95, or 105 linked nucleotides. In some embodiments, the length of a handle sequence in an sgRNA is about 30 to about 120 linked nucleotides. In some embodiments, the length of a handle sequence in an sgRNA is about 50 to about 105, about 50 to about 95, about 50 to about 73, about 50 to about 71, about 50 to about 70, or about 50 to about 69 linked nucleotides. In some embodiments, the length of a handle sequence in an sgRNA is 56 to 105 linked nucleotides, from 56 to 105 linked nucleotides, 66 to 105 linked nucleotides, 67 to 105 linked nucleotides, 68 to 105 linked nucleotides, 69 to 105 linked nucleotides, 70 to 105 linked nucleotides, 71 to 105 linked nucleotides, 72 to 105 linked nucleotides, 73 to 105 linked nucleotides, or 95 to 105 linked nucleotides. In some embodiments, the length of a handle sequence in an sgRNA is 40 to 70 nucleotides. In some embodiments, the length of a handle sequence in an sgRNA is 50, 56, 66, 67, 68, 69, 70, 71, 72, 73, 95, or 105 linked nucleotides.

In some embodiments, a handle sequence comprises a nucleotide sequence that is at least 65%, at least 70%, at least 75%, at least 80%, at least 85%, at least 90%, at least 95%, at least 97%, or at least 98%, at least 99%, or 100% identical to the sequence:

(SEQ ID NO: 490)
ACAGCUUAUUUGGAAGCUGAAAUGUGAGGUUUAUAACACUCACAAGAAUC
CUGAAAAAGGAUGCCAAAC.

### Exemplary Guide Nucleic Acids

In some embodiments, the guide nucleic acids disclosed herein comprise a spacer sequence that is at least 70%, at least 75%, at least 80%, at least 85%, at least 90%, at least 95%, at least 96%, at least 97%, at least 98%, at least 99%, or 100% identical to any one of the sequences as set forth in TABLES 1, 3, and 5, and a repeat sequence that is at least 70%, at least 75%, at least 80%, at least 85%, at least 90%, at least 95%, at least 96%, at least 97%, at least 98%, at least 99%, or 100% identical to any one of SEQ ID NOs: 16 or 38-43.

Exemplary guide nucleic acid sequences useful for systems, compositions and methods described herein are presented in are provided in TABLES 8-10. In some embodiments, the guide nucleic acid comprises a sequence that is at least 70%, at least 75%, at least 80%, at least 85%, at least 90%, at least 95%, or 100% identical to any one of the sequences of TABLES 8-10. In some embodiments, the guide nucleic acid consists of a sequence that is at least 70%, at least 75%, at least 80%, at least 85%, at least 90%, at least 95%, or 100% identical to any one of the sequences of TABLES 8-10. In some embodiments, the guide nucleic acids provided in TABLES 8-10 comprise an additional “G” at the 5′ end of the sequence. The combination of spacer and repeat sequences provided in TABLES 8-10 are provided for illustrative purposes. It should be understood that these guides can comprise any of the repeat sequences disclosed herein (e.g., any one of SEQ ID NOs: 16, and 38-43). For example, in some embodiments, the guide sequence comprises a spacer sequence selected from any one of SEQ ID NOs: 1-15, 67-72, 79-140, 207-208, 799-809, and 830-999 with a repeat sequence selected from any one of SEQ ID NOs: 16, and 38-43.

TABLE 8
Exemplary Guide Nucleic Acids Targeting APOC3
for CasPhi.12 Effector Proteins

| | | SEQ |
|---|---|---|
| Guide ID | Guide sequence (shown as RNA), 5′- 3′ | ID NO: |
| R15586 | AUAGAUUGCUCCUUACGAGGAGACUCCUUAACGGUGCUCCA | 17 |
| R15587 | AUAGAUUGCUCCUUACGAGGAGACACGGUGCUCCAGUAGUC | 18 |
| R15588 | AUAGAUUGCUCCUUACGAGGAGACAAGCAACCUACAGGGGC | 19 |
| R15589 | AUAGAUUGCUCCUUACGAGGAGACUCCAGCUUUAUUGGGAG | 20 |
| R15590 | AUAGAUUGCUCCUUACGAGGAGACGGGUAUUGAGGUCUCAG | 21 |
| R15591 | AUAGAUUGCUCCUUACGAGGAGACAGCAACCUACAGGGGCA | 22 |
| R15592 | AUAGAUUGCUCCUUACGAGGAGACAGGGAACUGAAGCCAUC | 23 |
| R15593 | AUAGAUUGCUCCUUACGAGGAGACUAAGCAACCUACAGGGG | 24 |
| R15594 | AUAGAUUGCUCCUUACGAGGAGACUUGUCCAGCUUUAUUGG | 25 |
| R15595 | AUAGAUUGCUCCUUACGAGGAGACCAGGGAACUGAAGCCAU | 26 |
| R15596 | AUAGAUUGCUCCUUACGAGGAGACCCUGAAAGACUACUGGA | 27 |
| R15597 | AUAGAUUGCUCCUUACGAGGAGACAAAGGGACAGUAUUCUC | 28 |
| R15598 | AUAGAUUGCUCCUUACGAGGAGACCUUAAAAGGGACAGUAU | 29 |
| R15599 | AUAGAUUGCUCCUUACGAGGAGACAGUUCCCUGAAAGACUA | 30 |
| R15600 | AUAGAUUGCUCCUUACGAGGAGACAUCCCUAGAGGCAGCUG | 31 |
| R17561 | AUAGAUUGCUCCUUACGAGGAGACCCCUCCCCAGAGGGCAU | 73 |
| R17562 | AUAGAUUGCUCCUUACGAGGAGACCCCCUCCCCAGAGGGCA | 74 |
| R17563 | AUAGAUUGCUCCUUACGAGGAGACCUUGCAGGAACAGAGGC | 75 |
| R17565 | AUAGAUUGCUCCUUACGAGGAGACCCUCAGGAGCUUCAGAG | 76 |
| R17566 | AUAGAUUGCUCCUUACGAGGAGACCUCAGGAGCUUCAGAGG | 77 |
| R17567 | AUAGAUUGCUCCUUACGAGGAGACUCAUGCCCUGCUCUGUU | 78 |
| R17564 | AUAGAUUGCUCCUUACGAGGAGACGUGGGACUGGGCUGGGG | 491 |
| n/a | AUUGCUCCUUACGAGGAGACCUUGCAGGAACAGAGGUGCC | 815 |
| n/a | AUUGCUCCUUACGAGGAGACCCUCAGGAGCUUCAGAGGCC | 816 |
| n/a | AUAGAUUGCUCCUUACGAGGAGACCCCAACUCUCCCGCCCG | 1400 |
| n/a | AUAGAUUGCUCCUUACGAGGAGACAGGCUUAGGGCUGGAGG | 1401 |
| n/a | AUAGAUUGCUCCUUACGAGGAGACCCCUCUCACCAGCCUCU | 1402 |
| n/a | AUAGAUUGCUCCUUACGAGGAGACAGGGCUUGGGGCUGGUG | 1403 |
| n/a | AUAGAUUGCUCCUUACGAGGAGACCUCCAAACACCCCCCAG | 1404 |
| n/a | AUAGAUUGCUCCUUACGAGGAGACGGGCUGGAGGAAGCCUU | 1405 |
| n/a | AUAGAUUGCUCCUUACGAGGAGACCCAACUCUCCCGCCCGC | 1406 |
| n/a | AUAGAUUGCUCCUUACGAGGAGACGCUGGACUGGACGGAGA | 1407 |
| n/a | AUAGAUUGCUCCUUACGAGGAGACUCUGCUCCAUCCCACCC | 1408 |
| n/a | AUAGAUUGCUCCUUACGAGGAGACCCCAGCGCCCUGGGUCC | 1409 |
| n/a | AUAGAUUGCUCCUUACGAGGAGACUGUGCCUUUACUCCAAA | 1410 |
| n/a | AUAGAUUGCUCCUUACGAGGAGACCUGCAUCUGGACACCCU | 1411 |
| n/a | AUAGAUUGCUCCUUACGAGGAGACCUAGAGCUAAGGAAGCC | 1412 |
| n/a | AUAGAUUGCUCCUUACGAGGAGACGCCCAGCGCCCUGGGUC | 1413 |
| n/a | AUAGAUUGCUCCUUACGAGGAGACCAGUGUGAAAGGCUGAG | 1414 |
| n/a | AUAGAUUGCUCCUUACGAGGAGACUUCAGGCUUAGGGCUGG | 1415 |
| n/a | AUAGAUUGCUCCUUACGAGGAGACGGGCCUCGAUCCCUCGC | 1416 |
| n/a | AUAGAUUGCUCCUUACGAGGAGACACUCCAAACACCCCCCA | 1417 |
| n/a | AUAGAUUGCUCCUUACGAGGAGACAGUCUGGUGGGUUUUCU | 1418 |
| n/a | AUAGAUUGCUCCUUACGAGGAGACCCCAAAGCUACACAGGG | 1419 |
| n/a | AUAGAUUGCUCCUUACGAGGAGACUGCUCCAUCCCACCCAC | 1420 |
| n/a | AUAGAUUGCUCCUUACGAGGAGACAUGUUCAGUCUGGUGGG | 1421 |
| n/a | AUAGAUUGCUCCUUACGAGGAGACCUGCUCCAUCCCACCCA | 1422 |
| n/a | AUAGAUUGCUCCUUACGAGGAGACAUCCCUAGAGGCAGCUG | 1423 |
| n/a | AUAGAUUGCUCCUUACGAGGAGACGACAGCCCAGUCCUACC | 1424 |
| n/a | AUAGAUUGCUCCUUACGAGGAGACGGGCUGGUGGAGGGAGG | 1425 |
| n/a | AUAGAUUGCUCCUUACGAGGAGACCUGAGCUCAUCUGGGCU | 1426 |
| n/a | AUAGAUUGCUCCUUACGAGGAGACGGCCUCGAUCCCUCGCC | 1427 |
| n/a | AUAGAUUGCUCCUUACGAGGAGACUCAAGUCUGAAGAAGCC | 1428 |
| n/a | AUAGAUUGCUCCUUACGAGGAGACCCCCUCUCACCAGCCUC | 1429 |
| n/a | AUAGAUUGCUCCUUACGAGGAGACUUCUCAAGUCUGAAGAA | 1430 |
| n/a | AUAGAUUGCUCCUUACGAGGAGACCCCCCUCAUUCUUCAGG | 1431 |
| n/a | AUAGAUUGCUCCUUACGAGGAGACGGCUGGGGGGUGUUUGG | 1432 |
| n/a | AUAGAUUGCUCCUUACGAGGAGACGGAAAUCCCUAGGAGAC | 1433 |
| n/a | AUAGAUUGCUCCUUACGAGGAGACAGAACAAGUGGGUGGCU | 1434 |
| n/a | AUAGAUUGCUCCUUACGAGGAGACUAUCAUCUCCAGGGCAG | 1435 |
| n/a | AUAGAUUGCUCCUUACGAGGAGACCAGGCCCCUCCCUCCAC | 1436 |
| n/a | AUAGAUUGCUCCUUACGAGGAGACCCUGGAGCAGCUGCCUC | 1437 |
| n/a | AUAGAUUGCUCCUUACGAGGAGACAGGUUAUGAUGAGGGGU | 1438 |
| n/a | AUAGAUUGCUCCUUACGAGGAGACCUGGCUGGGCUGGGCAG | 1439 |
| n/a | AUAGAUUGCUCCUUACGAGGAGACCUAGCUGACUGGCUCCC | 1440 |
| n/a | AUAGAUUGCUCCUUACGAGGAGACUUCAGACUUGAGAACAA | 1441 |
| n/a | AUAGAUUGCUCCUUACGAGGAGACGAGUAAAGGCACAGAAG | 1442 |
| n/a | AUAGAUUGCUCCUUACGAGGAGACGGCAAGUGACACCCCUC | 1443 |
| n/a | AUAGAUUGCUCCUUACGAGGAGACUGAUGAGGGGUGGGGGG | 1444 |
| n/a | AUAGAUUGCUCCUUACGAGGAGACUGGCCCUCUCCAGGCCU | 1445 |
| n/a | AUAGAUUGCUCCUUACGAGGAGACUUCAGGUUAUGAUGAGG | 1446 |
| n/a | AUAGAUUGCUCCUUACGAGGAGACUAUAUCAUCUCCAGGGC | 1447 |
| n/a | AUAGAUUGCUCCUUACGAGGAGACCCCUCCCCAGAGGGCAU | 1448 |
| n/a | AUAGAUUGCUCCUUACGAGGAGACCCCCUCUUCAUCCUCCU | 1449 |
| n/a | AUAGAUUGCUCCUUACGAGGAGACUCCAGGCUUGCUGGCUG | 1450 |
| n/a | AUAGAUUGCUCCUUACGAGGAGACCACACUGGAAUUUCAGG | 1451 |
| n/a | AUAGAUUGCUCCUUACGAGGAGACCCUGUCUGGGGUAGGAC | 1452 |
| n/a | AUAGAUUGCUCCUUACGAGGAGACGCUCUAGCAAGUGCUUC | 1453 |
| n/a | AUAGAUUGCUCCUUACGAGGAGACCUGGCCCUCUCCAGGCC | 1454 |
| n/a | AUAGAUUGCUCCUUACGAGGAGACAGACUUGAGAACAAGUG | 1455 |
| n/a | AUAGAUUGCUCCUUACGAGGAGACGGAGUAAAGGCACAGAA | 1456 |
| n/a | AUAGAUUGCUCCUUACGAGGAGACCCCCUCCCCAGAGGGCA | 1457 |
| n/a | AUAGAUUGCUCCUUACGAGGAGACGAGCCACUUCCAGCCCC | 1458 |
| n/a | AUAGAUUGCUCCUUACGAGGAGACCUUCCUAGCUGACUGGC | 1459 |
| n/a | AUAGAUUGCUCCUUACGAGGAGACCUCCAGCCCUAAGCCUG | 1460 |
| n/a | AUAGAUUGCUCCUUACGAGGAGACUGACCUGUUUUAUAUCA | 1461 |
| n/a | AUAGAUUGCUCCUUACGAGGAGACCAGCCCCACCCCCUGUG | 1462 |
| n/a | AUAGAUUGCUCCUUACGAGGAGACAGGCCCCUCCCUCCACC | 1463 |
| n/a | AUAGAUUGCUCCUUACGAGGAGACCUUAGCUCUAGCAAGUG | 1464 |
| n/a | AUAGAUUGCUCCUUACGAGGAGACGGGCAAGUGACACCCCU | 1465 |
| n/a | AUAGAUUGCUCCUUACGAGGAGACCCCUGUCUGGGGUAGGA | 1466 |
| n/a | AUAGAUUGCUCCUUACGAGGAGACGGUGAUUUCUGGCCCUC | 1467 |
| n/a | AUAGAUUGCUCCUUACGAGGAGACGGGUGAUUUCUGGCCCU | 1468 |
| n/a | AUAGAUUGCUCCUUACGAGGAGACACACUGGAAUUUCAGGC | 1469 |
| n/a | AUAGAUUGCUCCUUACGAGGAGACGACAUAGGCCAGGGGCC | 1470 |
| n/a | AUAGAUUGCUCCUUACGAGGAGACAUAUCAUCUCCAGGGCA | 1471 |
| n/a | AUAGAUUGCUCCUUACGAGGAGACAUCCUCCUCCCCUCCUC | 1472 |
| n/a | AUAGAUUGCUCCUUACGAGGAGACUCCCACUGAUAUUAGAU | 1473 |
| n/a | AUAGAUUGCUCCUUACGAGGAGACUGGCCCAUAGCCUCCCU | 1474 |
| n/a | AUAGAUUGCUCCUUACGAGGAGACCAGGCAGCUCUGCCACU | 1475 |
| n/a | AUAGAUUGCUCCUUACGAGGAGACCAGUAGAAUGGAAUGGG | 1476 |
| n/a | AUAGAUUGCUCCUUACGAGGAGACUAUUGGCUCCAGGAUGG | 1477 |
| n/a | AUAGAUUGCUCCUUACGAGGAGACCUUCCUCUCCUCCCCAG | 1478 |
| n/a | AUAGAUUGCUCCUUACGAGGAGACCAGUCCUGGGUAGGCAU | 1479 |
| n/a | AUAGAUUGCUCCUUACGAGGAGACCCUGGAGUAGCUAGCUG | 1480 |
| n/a | AUAGAUUGCUCCUUACGAGGAGACCCCAGCUUCUAGCCCCC | 1481 |
| n/a | AUAGAUUGCUCCUUACGAGGAGACUCCCUCCAGCUCUUUGU | 1482 |
| n/a | AUAGAUUGCUCCUUACGAGGAGACCCUUCCUUCCUCUCCUC | 1483 |
| n/a | AUAGAUUGCUCCUUACGAGGAGACCUCGCUAGGACUCAGUU | 1484 |
| n/a | AUAGAUUGCUCCUUACGAGGAGACAGAAAUCCCUCUGAGAU | 1485 |
| n/a | AUAGAUUGCUCCUUACGAGGAGACGUUUCUUCCCUUCCUUC | 1486 |
| n/a | AUAGAUUGCUCCUUACGAGGAGACUUCAGUCCUGGGUAGGC | 1487 |
| n/a | AUAGAUUGCUCCUUACGAGGAGACCCCAUGCUUUUCACGGC | 1488 |
| n/a | AUAGAUUGCUCCUUACGAGGAGACUUCCCUUCCUUCCUCUC | 1489 |
| n/a | AUAGAUUGCUCCUUACGAGGAGACCAUGCCCCCACACUGAC | 1490 |
| n/a | AUAGAUUGCUCCUUACGAGGAGACCUUUUCCUCGCUAGGAC | 1491 |
| n/a | AUAGAUUGCUCCUUACGAGGAGACCCUCGCUAGGACUCAGU | 1492 |
| n/a | AUAGAUUGCUCCUUACGAGGAGACUAUAUUGGCUCCAGGAU | 1493 |
| n/a | AUAGAUUGCUCCUUACGAGGAGACGCUCCAGGAUGGGACAG | 1494 |
| n/a | AUAGAUUGCUCCUUACGAGGAGACCACGGCCACCUCCGCCA | 1495 |
| n/a | AUAGAUUGCUCCUUACGAGGAGACUAGCCCCCCCCACACCA | 1496 |
| n/a | AUAGAUUGCUCCUUACGAGGAGACUUUCAGUCCUGGGUAGG | 1497 |
| n/a | AUAGAUUGCUCCUUACGAGGAGACGGCCCAUAGCCUCCCUU | 1498 |
| n/a | AUAGAUUGCUCCUUACGAGGAGACCUUCCCUUCCUUCCUCU | 1499 |
| n/a | AUAGAUUGCUCCUUACGAGGAGACGACCUCAGGCCUGCUUU | 1500 |
| n/a | AUAGAUUGCUCCUUACGAGGAGACCCCCAGCUUCUAGCCCC | 1501 |
| n/a | AUAGAUUGCUCCUUACGAGGAGACUUUCUUCCCUUCCUUCC | 1502 |
| n/a | AUAGAUUGCUCCUUACGAGGAGACUAGGGAUAAAACUGAGC | 1503 |
| n/a | AUAGAUUGCUCCUUACGAGGAGACAUAUUGGCUCCAGGAUG | 1504 |
| n/a | AUAGAUUGCUCCUUACGAGGAGACACGGCCACCUCCGCCAC | 1505 |
| n/a | AUAGAUUGCUCCUUACGAGGAGACACAGCCUAGAGCCAGUG | 1506 |
| n/a | AUAGAUUGCUCCUUACGAGGAGACACAGAAGCCACCUGAAA | 1507 |
| n/a | AUAGAUUGCUCCUUACGAGGAGACUCAGUCCUGGGUAGGCA | 1508 |
| n/a | AUAGAUUGCUCCUUACGAGGAGACUCACGGCCACCUCCGCC | 1509 |
| n/a | AUAGAUUGCUCCUUACGAGGAGACUCCUCGCUAGGACUCAG | 1510 |
| n/a | AUAGAUUGCUCCUUACGAGGAGACCUCCCACUGAUAUUAGA | 1511 |
| n/a | AUAGAUUGCUCCUUACGAGGAGACUUUUCCUCGCUAGGACU | 1512 |
| n/a | AUAGAUUGCUCCUUACGAGGAGACUGUGGGCUAGAUGGCUG | 1513 |
| n/a | AUAGAUUGCUCCUUACGAGGAGACGCCCAUAGCCUCCCUUU | 1514 |
| n/a | AUAGAUUGCUCCUUACGAGGAGACUAAUAGCUCAGAGCAAG | 1515 |
| n/a | AUAGAUUGCUCCUUACGAGGAGACAGUCCUGGGUAGGCAUG | 1516 |
| n/a | AUAGAUUGCUCCUUACGAGGAGACCAGCCUAGAGCCAGUGA | 1517 |
| n/a | AUAGAUUGCUCCUUACGAGGAGACCUCUCCUCCCCAGGGGC | 1518 |
| n/a | AUAGAUUGCUCCUUACGAGGAGACGAUAGAGAACUACAGUA | 1519 |
| n/a | AUAGAUUGCUCCUUACGAGGAGACGUGGCGGAGGUGGCCGU | 1520 |
| n/a | AUAGAUUGCUCCUUACGAGGAGACGGUCAUGCUGUCCCUUG | 1521 |
| n/a | AUAGAUUGCUCCUUACGAGGAGACGGUUCCUGGUGUGGGGG | 1522 |
| n/a | AUAGAUUGCUCCUUACGAGGAGACUCAGCAUAUUAGAGUAG | 1523 |
| n/a | AUAGAUUGCUCCUUACGAGGAGACUAUCCCUAGAAGCAGCU | 1524 |
| n/a | AUAGAUUGCUCCUUACGAGGAGACUCUAUCUAAUAUCAGUG | 1525 |
| n/a | AUAGAUUGCUCCUUACGAGGAGACCUAUACUCCACCUUCCA | 1526 |
| n/a | AUAGAUUGCUCCUUACGAGGAGACCCCAUUCCAUUCUACUG | 1527 |
| n/a | AUAGAUUGCUCCUUACGAGGAGACCUCCGUUGCUCCACAGU | 1528 |
| n/a | AUAGAUUGCUCCUUACGAGGAGACUCCCCUGUCUUUUCCUG | 1529 |
| n/a | AUAGAUUGCUCCUUACGAGGAGACAUCCCUAGAAGCAGCUA | 1530 |
| n/a | AUAGAUUGCUCCUUACGAGGAGACCACCCCACUUGGGGGGC | 1531 |
| n/a | AUAGAUUGCUCCUUACGAGGAGACUUUCUCAGCAUAUUAGA | 1532 |
| n/a | AUAGAUUGCUCCUUACGAGGAGACCAGGUGGCUUCUGUGAA | 1533 |
| n/a | AUAGAUUGCUCCUUACGAGGAGACCCCAGCUCACUGGGCCU | 1534 |
| n/a | AUAGAUUGCUCCUUACGAGGAGACCUCAGCAUAUUAGAGUA | 1535 |
| n/a | AUAGAUUGCUCCUUACGAGGAGACCAUUCUACUGGAAGGCU | 1536 |
| n/a | AUAGAUUGCUCCUUACGAGGAGACUCCUGUCUCACCGACCU | 1537 |
| n/a | AUAGAUUGCUCCUUACGAGGAGACGUAUCCAUGCCUACCCA | 1538 |
| n/a | AUAGAUUGCUCCUUACGAGGAGACAGGUGGCUUCUGUGAAG | 1539 |
| n/a | AUAGAUUGCUCCUUACGAGGAGACGGAUCACAGGUGGAGGU | 1540 |
| n/a | AUAGAUUGCUCCUUACGAGGAGACUUUAGCUUGCUCUGAGC | 1541 |
| n/a | AUAGAUUGCUCCUUACGAGGAGACGAAGCCUUUGGUAUCCA | 1542 |
| n/a | AUAGAUUGCUCCUUACGAGGAGACCUGUCUCACCGACCUCA | 1543 |
| n/a | AUAGAUUGCUCCUUACGAGGAGACUGUGAAGGAGCCUGUCA | 1544 |
| n/a | AUAGAUUGCUCCUUACGAGGAGACACUCUGCCCCCUCCCAC | 1545 |
| n/a | AUAGAUUGCUCCUUACGAGGAGACUCUCACUAAUCCCUGCC | 1546 |
| n/a | AUAGAUUGCUCCUUACGAGGAGACUACUGGAAGGCUUUCAG | 1547 |
| n/a | AUAGAUUGCUCCUUACGAGGAGACUAUACUCCACCUUCCAC | 1548 |
| n/a | AUAGAUUGCUCCUUACGAGGAGACGCCCAGCUCACUGGGCC | 1549 |
| n/a | AUAGAUUGCUCCUUACGAGGAGACCUCUGAGCUAUUAGAAG | 1550 |
| n/a | AUAGAUUGCUCCUUACGAGGAGACGGGGGCUGGGUCUACUG | 1551 |
| n/a | AUAGAUUGCUCCUUACGAGGAGACGGAUUCAUGACCCAGGA | 1552 |
| n/a | AUAGAUUGCUCCUUACGAGGAGACCCUGCUCAGUUUUAUCC | 1553 |
| n/a | AUAGAUUGCUCCUUACGAGGAGACCUCAACUCCUCUGGCAG | 1554 |
| n/a | AUAGAUUGCUCCUUACGAGGAGACCUGCCUCAGGCUCUGGU | 1555 |
| n/a | AUAGAUUGCUCCUUACGAGGAGACUGUGCCCGCUGUCCCAU | 1556 |
| n/a | AUAGAUUGCUCCUUACGAGGAGACUCCUUCUCUCACUAAUC | 1557 |
| n/a | AUAGAUUGCUCCUUACGAGGAGACGCUUGCUCUGAGCUAUU | 1558 |
| n/a | AUAGAUUGCUCCUUACGAGGAGACUCAACUCCUCUGGCAGA | 1559 |
| n/a | AUAGAUUGCUCCUUACGAGGAGACCCUGUCUCACCGACCUC | 1560 |
| n/a | AUAGAUUGCUCCUUACGAGGAGACCUCCACAGUGGCACCAC | 1561 |
| n/a | AUAGAUUGCUCCUUACGAGGAGACUCCCUAGAAGCAGCUAG | 1562 |
| n/a | AUAGAUUGCUCCUUACGAGGAGACGGACUCAUGGUCUCCAC | 1563 |
| n/a | AUAGAUUGCUCCUUACGAGGAGACAGCUUGCUCUGAGCUAU | 1564 |
| n/a | AUAGAUUGCUCCUUACGAGGAGACGGUAUCCAUGCCUACCC | 1565 |
| n/a | AUAGAUUGCUCCUUACGAGGAGACCUGGUGUGGGGGGGGCU | 1566 |
| n/a | AUAGAUUGCUCCUUACGAGGAGACCACUGGGUAGUGGCAGA | 1567 |
| n/a | AUAGAUUGCUCCUUACGAGGAGACUGCAGAGUAUUUCUAUA | 1568 |
| n/a | AUAGAUUGCUCCUUACGAGGAGACGAGUAGAUGUCCCGUUC | 1569 |

TABLE 9
Exemplary Guide Nucleic Acids Targeting PCSK9
for CasPhi.12 Effector Proteins

| Guide | | SEQ ID |
|---|---|---|
| ID | Guide sequence (shown as RNA), 5′- 3′ | NO: |
| R14046 | AUAGAUUGCUCCUUACGAGGAGACCGGUGGGGAGGACUGUG | 141 |
| R14049 | AUAGAUUGCUCCUUACGAGGAGACCGUUCCGAGGAGGACGG | 142 |
| R14050 | AUAGAUUGCUCCUUACGAGGAGACCGAGGAGGACGGCCUGG | 143 |
| R14051 | AUAGAUUGCUCCUUACGAGGAGACGCGCAGCGGUGGAAGGU | 144 |
| R14052 | AUAGAUUGCUCCUUACGAGGAGACAGCACCACCACGUAGGU | 145 |
| R14053 | AUAGAUUGCUCCUUACGAGGAGACGGCCUGCAGGCGGCGGG | 146 |
| R14054 | AUAGAUUGCUCCUUACGAGGAGACGUGAGGUAUCCCCGGCG | 147 |
| R14056 | AUAGAUUGCUCCUUACGAGGAGACUUCCUGGCUUCCUGGUG | 148 |
| R14057 | AUAGAUUGCUCCUUACGAGGAGACACCAGGAAGCCAGGAAG | 149 |
| R14058 | AUAGAUUGCUCCUUACGAGGAGACCUGGCUUCCUGGUGAAG | 150 |
| R14059 | AUAGAUUGCUCCUUACGAGGAGACCUGGUGAAGAUGAGUGG | 151 |
| R14060 | AUAGAUUGCUCCUUACGAGGAGACCCCCAUGUCGACUACAU | 152 |
| R14065 | AUAGAUUGCUCCUUACGAGGAGACAUCCGCCCGGUACCGUG | 153 |
| R14066 | AUAGAUUGCUCCUUACGAGGAGACCCGGUGGUCACUCUGUA | 154 |
| R14067 | AUAGAUUGCUCCUUACGAGGAGACCCCGGUGGUCACUCUGU | 155 |
| R14070 | AUAGAUUGCUCCUUACGAGGAGACGCCACGCCGGCAUCCCG | 156 |
| R14072 | AUAGAUUGCUCCUUACGAGGAGACGCAGUUGAGCACGCGCA | 157 |
| R14073 | AUAGAUUGCUCCUUACGAGGAGACCCUUGGCAGUUGAGCAC | 158 |
| R14074 | AUAGAUUGCUCCUUACGAGGAGACCCGAAUAAACUCCAGGC | 159 |
| R14075 | AUAGAUUGCUCCUUACGAGGAGACUCCGAAUAAACUCCAGG | 160 |
| R14076 | AUAGAUUGCUCCUUACGAGGAGACAUUCGGAAAAGCCAGCU | 161 |
| R14077 | AUAGAUUGCUCCUUACGAGGAGACUUCGGAAAAGCCAGCUG | 162 |
| R14078 | AUAGAUUGCUCCUUACGAGGAGACGGAAAAGCCAGCUGGUC | 163 |
| R14079 | AUAGAUUGCUCCUUACGAGGAGACUGCUGCUGCCCCUGGCG | 164 |
| R14081 | AUAGAUUGCUCCUUACGAGGAGACAACGCCGCCUGCCAGCG | 165 |
| R14082 | AUAGAUUGCUCCUUACGAGGAGACCCGGCAGCGGUGACCAG | 166 |
| R14083 | AUAGAUUGCUCCUUACGAGGAGACCGGGACGAUGCCUGCCU | 167 |
| R14084 | AUAGAUUGCUCCUUACGAGGAGACGUGGCCCCAACUGUGAU | 168 |
| R14086 | AUAGAUUGCUCCUUACGAGGAGACGUCCCCAAAGUCCCCAG | 169 |
| R14087 | AUAGAUUGCUCCUUACGAGGAGACGGGGACCAACUUUGGCC | 170 |
| R14088 | AUAGAUUGCUCCUUACGAGGAGACGGGACCAACUUUGGCCG | 171 |
| R14089 | AUAGAUUGCUCCUUACGAGGAGACGGCCGCUGUGUGGACCU | 172 |
| R14090 | AUAGAUUGCUCCUUACGAGGAGACGCCGCUGUGUGGACCUC | 173 |
| R14091 | AUAGAUUGCUCCUUACGAGGAGACGCCCCAGGGGAGGACAU | 174 |
| R14092 | AUAGAUUGCUCCUUACGAGGAGACCCCCAGGGGAGGACAUC | 175 |
| R14093 | AUAGAUUGCUCCUUACGAGGAGACGUGCCUCCAGCGACUGC | 176 |
| R14096 | AUAGAUUGCUCCUUACGAGGAGACCAGCCAUGAUGCUGUCU | 177 |
| R14097 | AUAGAUUGCUCCUUACGAGGAGACAGGCAGAGACUGAUCCA | 178 |
| R14098 | AUAGAUUGCUCCUUACGAGGAGACGCAGAGAAGUGGAUCAG | 179 |
| R14099 | AUAGAUUGCUCCUUACGAGGAGACGGCAGAGAAGUGGAUCA | 180 |
| R14100 | AUAGAUUGCUCCUUACGAGGAGACUCUGCCAAAGAUGUCAU | 181 |
| R14101 | AUAGAUUGCUCCUUACGAGGAGACAUGACAUCUUUGGCAGA | 182 |
| R14102 | AUAGAUUGCUCCUUACGAGGAGACCCUGAGGACCAGCGGGU | 183 |
| R14103 | AUAGAUUGCUCCUUACGAGGAGACGGGGUCAGUACCCGCUG | 184 |
| R14104 | AUAGAUUGCUCCUUACGAGGAGACGCAGCUGUUUUGCAGGA | 185 |
| R14108 | AUAGAUUGCUCCUUACGAGGAGACCCACUCCUGGAGAAACU | 186 |
| R14109 | AUAGAUUGCUCCUUACGAGGAGACCUCCAGGAGUGGGAAGC | 187 |
| R14110 | AUAGAUUGCUCCUUACGAGGAGACUCCAGGAGUGGGAAGCG | 188 |
| R14112 | AUAGAUUGCUCCUUACGAGGAGACUGGGGGUGAGGGUGUCU | 189 |
| R14113 | AUAGAUUGCUCCUUACGAGGAGACGGGGGUGAGGGUGUCUA | 190 |
| R14114 | AUAGAUUGCUCCUUACGAGGAGACGGGGUGAGGGUGUCUAC | 191 |
| R14115 | AUAGAUUGCUCCUUACGAGGAGACCCAGGUGCUGCCUGCUA | 192 |
| R14117 | AUAGAUUGCUCCUUACGAGGAGACUGGGUGCCAAGGUCCUC | 193 |
| R14118 | AUAGAUUGCUCCUUACGAGGAGACGCACCCACAAGCCGCCU | 194 |
| R14119 | AUAGAUUGCUCCUUACGAGGAGACGGCUGACCUCGUGGCCU | 195 |
| R14123 | AUAGAUUGCUCCUUACGAGGAGACCAUUCCAGACCUGGGGC | 196 |
| R14124 | AUAGAUUGCUCCUUACGAGGAGACGCAUUCCAGACCUGGGG | 197 |
| R14125 | AUAGAUUGCUCCUUACGAGGAGACACUUUGCAUUCCAGACC | 198 |
| R14126 | AUAGAUUGCUCCUUACGAGGAGACCAUGCUCCUUGACUUUG | 199 |
| R14127 | AUAGAUUGCUCCUUACGAGGAGACUCGUGGCCUGUGAGGAC | 200 |
| R14130 | AUAGAUUGCUCCUUACGAGGAGACUUCGCUGGUGCUGCCUG | 201 |
| R14131 | AUAGAUUGCUCCUUACGAGGAGACCCAUCUGCUGCCGGAGC | 202 |
| n/a | AUAGAUUGCUCCUUACGAGGAGACGAGCAACGGCGGAAGGU | 492 |
| n/a | mA*mU*mA*GAUUGCUCCUUACGAGGAGACGAGCAACGGCGG | 493 |
| | AAmG*mG*mU | |
| PL34711 | AUUGCUCCUUACGAGGAGACACCCACCUGUGCCGCGGCGA | 810 |
| PL34712 | AUUGCUCCUUACGAGGAGACCAUGGGGCCAGGAUCCGUGG | 811 |
| PL34713 | AUUGCUCCUUACGAGGAGACUGCAGGCCUUGAAGUUGCCC | 812 |
| PL34714 | AUUGCUCCUUACGAGGAGACGUCGAGCAGGCCAGCAAGUG | 813 |
| PL34715 | AUUGCUCCUUACGAGGAGACCUCCCAGGCCUGGAGUUUAU | 814 |
| PL34722 | AUUGCUCCUUACGAGGAGACGAAAGACGGAGGCAGCCUGG | 820 |

TABLE 10
Exemplary Guide Nucleic Acids Targeting ANGPTL3 for CasPhi.12
Effector Proteins

| Guide | | SEQ ID |
|---|---|---|
| ID | Guide sequence (shown as RNA), 5′- 3′ | NO: |
| PL34718 | AUUGCUCCUUACGAGGAGACUACUUACUUUAAGUGAAGUU | 817 |
| PL34719 | AUUGCUCCUUACGAGGAGACUAUCAGCUCAGAAGGACUAG | 818 |
| PL34720 | AUUGCUCCUUACGAGGAGACAUUCUAGGCAUUCCUGCUGA | 819 |

In some embodiments, the guide nucleic acids disclosed herein comprise a spacer sequence that is at least 70%, at least 75%, at least 80%, at least 85%, at least 90%, at least 95%, at least 96%, at least 97%, at least 98%, at least 99%, or 100% identical to any one of the sequences as set forth in TABLES 2, 4, and 6, a repeat sequence that is at least 70%, at least 75%, at least 80%, at least 85%, at least 90%, at least 95%, or 100% identical to SEQ ID NO: 488, and an intermediary sequence that is at least 70%, at least 75%, at least 80%, at least 85%, at least 90%, at least 95%, at least 96%, at least 97%, at least 98%, at least 99%, or 100% identical to SEQ ID NO: 489.

Exemplary guide nucleic acid sequences useful for systems, compositions and methods described herein are presented in TABLES 11-13. In some embodiments, the guide nucleic acid comprises a sequence that is at least 70%, at least 75%, at least 80%, at least 85%, at least 90%, at least 95%, or 100% identical to any one of the sequences of TABLES 11-13. In some embodiments, the guide nucleic acid consists of a sequence that is at least 70%, at least 75%, at least 80%, at least 85%, at least 90%, at least 95%, or 100% identical to any one of the sequences of TABLES 11-13. In some embodiments, the guide nucleic acids provided in TABLES 11-13 comprise an additional “G” at the 5′ end of the sequence. In some embodiments, the guide sequence comprises a spacer sequence selected from any one of SEQ ID NOs: 209-487, 822-825, 1000-1399, 1970-2026, and 2084-2086 with repeat sequence SEQ ID NOs: 488.

TABLE 11
Exemplary Guide Nucleic Acids Targeting APOC3 for CasM.265466
Effector Proteins

| | | SEQ ID |
|---|---|---|
| Guide ID | Guide sequence (shown as RNA), 5′-3′ | NO: |
| R17774 | ACAGCUUAUUUGGAAGCUGAAAUGUGAGGUUUAUAACA | 494 |
| | CUCACAAGAAUCCUGAAAAAGGAUGCCAAACCCUAGAG | |
| | GCAGCUGCUCCAG | |
| R17775 | ACAGCUUAUUUGGAAGCUGAAAUGUGAGGUUUAUAACA | 495 |
| | CUCACAAGAAUCCUGAAAAAGGAUGCCAAACCAUCCCU | |
| | AGAGGCAGCUGCU | |
| R17776 | ACAGCUUAUUUGGAAGCUGAAAUGUGAGGUUUAUAACA | 496 |
| | CUCACAAGAAUCCUGAAAAAGGAUGCCAAACAUCCCUA | |
| | GAGGCAGCUGCUC | |
| R17525 | ACAGCUUAUUUGGAAGCUGAAAUGUGAGGUUUAUAACA | 497 |
| | CUCACAAGAAUCCUGAAAAAGGAUGCCAAACCAGCCCC | |
| | GGGUACUCCUUGU | |
| R17526 | ACAGCUUAUUUGGAAGCUGAAAUGUGAGGUUUAUAACA | 498 |
| | CUCACAAGAAUCCUGAAAAAGGAUGCCAAACCUGGGCC | |
| | ACCUGGGACUCCU | |
| R17527 | ACAGCUUAUUUGGAAGCUGAAAUGUGAGGUUUAUAACA | 499 |
| | CUCACAAGAAUCCUGAAAAAGGAUGCCAAACCAUCCUU | |
| | GGCGGUCUUGGUG | |
| R17777 | ACAGCUUAUUUGGAAGCUGAAAUGUGAGGUUUAUAACA | 500 |
| | CUCACAAGAAUCCUGAAAAAGGAUGCCAAACCCAGGUG | |
| | GCCCAGCAGGCCA | |
| R17778 | ACAGCUUAUUUGGAAGCUGAAAUGUGAGGUUUAUAACA | 501 |
| | CUCACAAGAAUCCUGAAAAAGGAUGCCAAACCAGGAGU | |
| | CCCAGGUGGCCCA | |
| R17779 | ACAGCUUAUUUGGAAGCUGAAAUGUGAGGUUUAUAACA | 502 |
| | CUCACAAGAAUCCUGAAAAAGGAUGCCAAACAGUGCAU | |
| | CCUUGGCGGUCUU | |
| R17528 | ACAGCUUAUUUGGAAGCUGAAAUGUGAGGUUUAUAACA | 503 |
| | CUCACAAGAAUCCUGAAAAAGGAUGCCAAACGGUUGCU | |
| | UAAAAGGGACAGU | |
| R17539 | ACAGCUUAUUUGGAAGCUGAAAUGUGAGGUUUAUAACA | 504 |
| | CUCACAAGAAUCCUGAAAAAGGAUGCCAAACAGCAACC | |
| | UACAGGGGCAGCC | |
| R17529 | ACAGCUUAUUUGGAAGCUGAAAUGUGAGGUUUAUAACA | 505 |
| | CUCACAAGAAUCCUGAAAAAGGAUGCCAAACGGUGACC | |
| | GAUGGCUUCAGUU | |
| R17530 | ACAGCUUAUUUGGAAGCUGAAAUGUGAGGUUUAUAACA | 506 |
| | CUCACAAGAAUCCUGAAAAAGGAUGCCAAACCCCCUGU | |
| | AGGUUGCUUAAAA | |
| R17531 | ACAGCUUAUUUGGAAGCUGAAAUGUGAGGUUUAUAACA | 507 |
| | CUCACAAGAAUCCUGAAAAAGGAUGCCAAACGAGCACC | |
| | GUUAAGGACAAGU | |
| R17532 | ACAGCUUAUUUGGAAGCUGAAAUGUGAGGUUUAUAACA | 508 |
| | CUCACAAGAAUCCUGAAAAAGGAUGCCAAACGCUUCAG | |
| | UUCCCUGAAAGAC | |
| R17533 | ACAGCUUAUUUGGAAGCUGAAAUGUGAGGUUUAUAACA | 509 |
| | CUCACAAGAAUCCUGAAAAAGGAUGCCAAACAGACCUC | |
| | AAUACCCCAAGUC | |
| R17534 | ACAGCUUAUUUGGAAGCUGAAAUGUGAGGUUUAUAACA | 510 |
| | CUCACAAGAAUCCUGAAAAAGGAUGCCAAACCUUAAAA | |
| | GGGACAGUAUUCU | |
| R17535 | ACAGCUUAUUUGGAAGCUGAAAUGUGAGGUUUAUAACA | 511 |
| | CUCACAAGAAUCCUGAAAAAGGAUGCCAAACAAGCUGG | |
| | ACAAGAAGCUGCU | |
| R17536 | ACAGCUUAUUUGGAAGCUGAAAUGUGAGGUUUAUAACA | 512 |
| | CUCACAAGAAUCCUGAAAAAGGAUGCCAAACCCUGAGA | |
| | CCUCAAUACCCCA | |
| R17537 | ACAGCUUAUUUGGAAGCUGAAAUGUGAGGUUUAUAACA | 513 |
| | CUCACAAGAAUCCUGAAAAAGGAUGCCAAACACCGAUG | |
| | GCUUCAGUUCCCU | |
| R17538 | ACAGCUUAUUUGGAAGCUGAAAUGUGAGGUUUAUAACA | 514 |
| | CUCACAAGAAUCCUGAAAAAGGAUGCCAAACAAAGGGA | |
| | CAGUAUUCUCAGU | |
| R17540 | ACAGCUUAUUUGGAAGCUGAAAUGUGAGGUUUAUAACA | 515 |
| | CUCACAAGAAUCCUGAAAAAGGAUGCCAAACAAGCCAU | |
| | CGGUCACCCAGCC | |
| R17541 | ACAGCUUAUUUGGAAGCUGAAAUGUGAGGUUUAUAACA | 516 |
| | CUCACAAGAAUCCUGAAAAAGGAUGCCAAACUCCCUUU | |
| | UAAGCAACCUACA | |
| R17542 | ACAGCUUAUUUGGAAGCUGAAAUGUGAGGUUUAUAACA | 517 |
| | CUCACAAGAAUCCUGAAAAAGGAUGCCAAACUCCUUAA | |
| | CGGUGCUCCAGUA | |
| R17543 | ACAGCUUAUUUGGAAGCUGAAAUGUGAGGUUUAUAACA | 518 |
| | CUCACAAGAAUCCUGAAAAAGGAUGCCAAACAGAAUAC | |
| | UGUCCCUUUUAAG | |
| R17544 | ACAGCUUAUUUGGAAGCUGAAAUGUGAGGUUUAUAACA | 519 |
| | CUCACAAGAAUCCUGAAAAAGGAUGCCAAACCCUGGAG | |
| | GGGGGCCAGGCAU | |
| R17545 | ACAGCUUAUUUGGAAGCUGAAAUGUGAGGUUUAUAACA | 520 |
| | CUCACAAGAAUCCUGAAAAAGGAUGCCAAACACGGUGC | |
| | UCCAGUAGUCUUU | |
| R17546 | ACAGCUUAUUUGGAAGCUGAAAUGUGAGGUUUAUAACA | 521 |
| | CUCACAAGAAUCCUGAAAAAGGAUGCCAAACGCAGCUU | |
| | CUUGUCCAGCUUU | |
| R17547 | ACAGCUUAUUUGGAAGCUGAAAUGUGAGGUUUAUAACA | 522 |
| | CUCACAAGAAUCCUGAAAAAGGAUGCCAAACGUCUUUC | |
| | AGGGAACUGAAGC | |
| R17548 | ACAGCUUAUUUGGAAGCUGAAAUGUGAGGUUUAUAACA | 523 |
| | CUCACAAGAAUCCUGAAAAAGGAUGCCAAACGGGUAUU | |
| | GAGGUCUCAGGCA | |
| R17549 | ACAGCUUAUUUGGAAGCUGAAAUGUGAGGUUUAUAACA | 524 |
| | CUCACAAGAAUCCUGAAAAAGGAUGCCAAACCUCCAGU | |
| | AGUCUUUCAGGGA | |
| R17550 | ACAGCUUAUUUGGAAGCUGAAAUGUGAGGUUUAUAACA | 525 |
| | CUCACAAGAAUCCUGAAAAAGGAUGCCAAACGACUUGG | |
| | GGUAUUGAGGUCU | |
| R17551 | ACAGCUUAUUUGGAAGCUGAAAUGUGAGGUUUAUAACA | 526 |
| | CUCACAAGAAUCCUGAAAAAGGAUGCCAAACCUGUCCC | |
| | UUUUAAGCAACCU | |
| R17780 | ACAGCUUAUUUGGAAGCUGAAAUGUGAGGUUUAUAACA | 527 |
| | CUCACAAGAAUCCUGAAAAAGGAUGCCAAACCCCUGAA | |
| | AGACUACUGGAGC | |
| R17781 | ACAGCUUAUUUGGAAGCUGAAAUGUGAGGUUUAUAACA | 528 |
| | CUCACAAGAAUCCUGAAAAAGGAUGCCAAACUAGGUUG | |
| | CUUAAAAGGGACA | |
| R17782 | ACAGCUUAUUUGGAAGCUGAAAUGUGAGGUUUAUAACA | 529 |
| | CUCACAAGAAUCCUGAAAAAGGAUGCCAAACCCUGAAA | |
| | GACUACUGGAGCA | |
| R17783 | ACAGCUUAUUUGGAAGCUGAAAUGUGAGGUUUAUAACA | 530 |
| | CUCACAAGAAUCCUGAAAAAGGAUGCCAAACCUGGAGC | |
| | ACCGUUAAGGACA | |
| R17784 | ACAGCUUAUUUGGAAGCUGAAAUGUGAGGUUUAUAACA | 531 |
| | CUCACAAGAAUCCUGAAAAAGGAUGCCAAACAAAGACU | |
| | ACUGGAGCACCGU | |
| R17785 | ACAGCUUAUUUGGAAGCUGAAAUGUGAGGUUUAUAACA | 532 |
| | CUCACAAGAAUCCUGAAAAAGGAUGCCAAACGCUUAAA | |
| | AGGGACAGUAUUC | |
| R17786 | ACAGCUUAUUUGGAAGCUGAAAUGUGAGGUUUAUAACA | 533 |
| | CUCACAAGAAUCCUGAAAAAGGAUGCCAAACAGUUCCC | |
| | UGAAAGACUACUG | |
| R17787 | ACAGCUUAUUUGGAAGCUGAAAUGUGAGGUUUAUAACA | 534 |
| | CUCACAAGAAUCCUGAAAAAGGAUGCCAAACCAGUUCC | |
| | CUGAAAGACUACU | |
| R17788 | ACAGCUUAUUUGGAAGCUGAAAUGUGAGGUUUAUAACA | 535 |
| | CUCACAAGAAUCCUGAAAAAGGAUGCCAAACAAUACCC | |
| | CAAGUCCACCUGC | |
| R17789 | ACAGCUUAUUUGGAAGCUGAAAUGUGAGGUUUAUAACA | 536 |
| | CUCACAAGAAUCCUGAAAAAGGAUGCCAAACAUGCCUG | |
| | GCCCCCCUCCAGG | |
| R17790 | ACAGCUUAUUUGGAAGCUGAAAUGUGAGGUUUAUAACA | 537 |
| | CUCACAAGAAUCCUGAAAAAGGAUGCCAAACCAGGGCU | |
| | GCCCCUGUAGGUU | |
| R17791 | ACAGCUUAUUUGGAAGCUGAAAUGUGAGGUUUAUAACA | 538 |
| | CUCACAAGAAUCCUGAAAAAGGAUGCCAAACAAAAGGG | |
| | ACAGUAUUCUCAG | |
| R17792 | ACAGCUUAUUUGGAAGCUGAAAUGUGAGGUUUAUAACA | 539 |
| | CUCACAAGAAUCCUGAAAAAGGAUGCCAAACCCAAUAA | |
| | AGCUGGACAAGAA | |
| R17793 | ACAGCUUAUUUGGAAGCUGAAAUGUGAGGUUUAUAACA | 540 |
| | CUCACAAGAAUCCUGAAAAAGGAUGCCAAACAGGGAAC | |
| | UGAAGCCAUCGGU | |
| R17794 | ACAGCUUAUUUGGAAGCUGAAAUGUGAGGUUUAUAACA | 541 |
| | CUCACAAGAAUCCUGAAAAAGGAUGCCAAACUUAAGCA | |
| | ACCUACAGGGGCA | |
| R17795 | ACAGCUUAUUUGGAAGCUGAAAUGUGAGGUUUAUAACA | 542 |
| | CUCACAAGAAUCCUGAAAAAGGAUGCCAAACCUUGUCC | |
| | AGCUUUAUUGGGA | |
| R17796 | ACAGCUUAUUUGGAAGCUGAAAUGUGAGGUUUAUAACA | 543 |
| | CUCACAAGAAUCCUGAAAAAGGAUGCCAAACCCUUUUA | |
| | AGCAACCUACAGG | |
| R17797 | ACAGCUUAUUUGGAAGCUGAAAUGUGAGGUUUAUAACA | 544 |
| | CUCACAAGAAUCCUGAAAAAGGAUGCCAAACUUUCAGG | |
| | GAACUGAAGCCAU | |
| R17798 | ACAGCUUAUUUGGAAGCUGAAAUGUGAGGUUUAUAACA | 545 |
| | CUCACAAGAAUCCUGAAAAAGGAUGCCAAACCUUAACG | |
| | GUGCUCCAGUAGU | |
| R17799 | ACAGCUUAUUUGGAAGCUGAAAUGUGAGGUUUAUAACA | 546 |
| | CUCACAAGAAUCCUGAAAAAGGAUGCCAAACCAGUAGU | |
| | CUUUCAGGGAACU | |
| R17800 | ACAGCUUAUUUGGAAGCUGAAAUGUGAGGUUUAUAACA | 547 |
| | CUCACAAGAAUCCUGAAAAAGGAUGCCAAACUCAGGGA | |
| | ACUGAAGCCAUCG | |
| R17801 | ACAGCUUAUUUGGAAGCUGAAAUGUGAGGUUUAUAACA | 548 |
| | CUCACAAGAAUCCUGAAAAAGGAUGCCAAACAACGGUG | |
| | CUCCAGUAGUCUU | |
| R17802 | ACAGCUUAUUUGGAAGCUGAAAUGUGAGGUUUAUAACA | 549 |
| | CUCACAAGAAUCCUGAAAAAGGAUGCCAAACUAAGCAA | |
| | CCUACAGGGGCAG | |
| R17803 | ACAGCUUAUUUGGAAGCUGAAAUGUGAGGUUUAUAACA | 550 |
| | CUCACAAGAAUCCUGAAAAAGGAUGCCAAACUUGUCCA | |
| | GCUUUAUUGGGAG | |
| R17804 | ACAGCUUAUUUGGAAGCUGAAAUGUGAGGUUUAUAACA | 551 |
| | CUCACAAGAAUCCUGAAAAAGGAUGCCAAACGGGGUAU | |
| | UGAGGUCUCAGGC | |
| R17805 | ACAGCUUAUUUGGAAGCUGAAAUGUGAGGUUUAUAACA | 552 |
| | CUCACAAGAAUCCUGAAAAAGGAUGCCAAACCAGGGAA | |
| | CUGAAGCCAUCGG | |
| R17806 | ACAGCUUAUUUGGAAGCUGAAAUGUGAGGUUUAUAACA | 553 |
| | CUCACAAGAAUCCUGAAAAAGGAUGCCAAACGUCCUUA | |
| | ACGGUGCUCCAGU | |
| R17807 | ACAGCUUAUUUGGAAGCUGAAAUGUGAGGUUUAUAACA | 554 |
| | CUCACAAGAAUCCUGAAAAAGGAUGCCAAACAAGCAAC | |
| | CUACAGGGGCAGC | |
| R17552 | ACAGCUUAUUUGGAAGCUGAAAUGUGAGGUUUAUAACA | 555 |
| | CUCACAAGAAUCCUGAAAAAGGAUGCCAAACGAGCAGC | |
| | UGCCUCUAGGGAU | |
| R17553 | ACAGCUUAUUUGGAAGCUGAAAUGUGAGGUUUAUAACA | 556 |
| | CUCACAAGAAUCCUGAAAAAGGAUGCCAAACCCUGGAG | |
| | CAGCUGCCUCUAG | |
| R17808 | ACAGCUUAUUUGGAAGCUGAAAUGUGAGGUUUAUAACA | 557 |
| | CUCACAAGAAUCCUGAAAAAGGAUGCCAAACACCUGGA | |
| | GCAGCUGCCUCUA | |
| R17809 | ACAGCUUAUUUGGAAGCUGAAAUGUGAGGUUUAUAACA | 558 |
| | CUCACAAGAAUCCUGAAAAAGGAUGCCAAACCCCAGAG | |
| | GGCAUUACCUGGA | |
| R17810 | ACAGCUUAUUUGGAAGCUGAAAUGUGAGGUUUAUAACA | 559 |
| | CUCACAAGAAUCCUGAAAAAGGAUGCCAAACCCCUCCC | |
| | CAGAGGGCAUUAC | |
| R17811 | ACAGCUUAUUUGGAAGCUGAAAUGUGAGGUUUAUAACA | 560 |
| | CUCACAAGAAUCCUGAAAAAGGAUGCCAAACCCCCUCC | |
| | CCAGAGGGCAUUA | |
| R17812 | ACAGCUUAUUUGGAAGCUGAAAUGUGAGGUUUAUAACA | 561 |
| | CUCACAAGAAUCCUGAAAAAGGAUGCCAAACUCCCCUC | |
| | CCCAGAGGGCAUU | |
| R17813 | ACAGCUUAUUUGGAAGCUGAAAUGUGAGGUUUAUAACA | 562 |
| | CUCACAAGAAUCCUGAAAAAGGAUGCCAAACUUUCCCC | |
| | UCCCCAGAGGGCA | |
| R17814 | ACAGCUUAUUUGGAAGCUGAAAUGUGAGGUUUAUAACA | 563 |
| | CUCACAAGAAUCCUGAAAAAGGAUGCCAAACCUCUUUC | |
| | CCCUCCCCAGAGG | |
| R17815 | ACAGCUUAUUUGGAAGCUGAAAUGUGAGGUUUAUAACA | 564 |
| | CUCACAAGAAUCCUGAAAAAGGAUGCCAAACCCCUCCU | |
| | CUUUCCCCUCCCC | |
| R17816 | ACAGCUUAUUUGGAAGCUGAAAUGUGAGGUUUAUAACA | 565 |
| | CUCACAAGAAUCCUGAAAAAGGAUGCCAAACCUCCCCU | |
| | CCUCUUUCCCCUC | |
| R17554 | ACAGCUUAUUUGGAAGCUGAAAUGUGAGGUUUAUAACA | 566 |
| | CUCACAAGAAUCCUGAAAAAGGAUGCCAAACCUCUUUC | |
| | CUCAGGAGCUUCA | |
| R17555 | ACAGCUUAUUUGGAAGCUGAAAUGUGAGGUUUAUAACA | 567 |
| | CUCACAAGAAUCCUGAAAAAGGAUGCCAAACAAGCUCC | |
| | UGAGGAAAGAGCA | |
| R17817 | ACAGCUUAUUUGGAAGCUGAAAUGUGAGGUUUAUAACA | 568 |
| | CUCACAAGAAUCCUGAAAAAGGAUGCCAAACAGCCCUG | |
| | CUCUUUCCUCAGG | |
| R17818 | ACAGCUUAUUUGGAAGCUGAAAUGUGAGGUUUAUAACA | 569 |
| | CUCACAAGAAUCCUGAAAAAGGAUGCCAAACUUUCCUC | |
| | AGGAGCUUCAGAG | |
| R17819 | ACAGCUUAUUUGGAAGCUGAAAUGUGAGGUUUAUAACA | 570 |
| | CUCACAAGAAUCCUGAAAAAGGAUGCCAAACUCCUCAG | |
| | GAGCUUCAGAGGC | |
| R17820 | ACAGCUUAUUUGGAAGCUGAAAUGUGAGGUUUAUAACA | 571 |
| | CUCACAAGAAUCCUGAAAAAGGAUGCCAAACCCUCAGG | |
| | AGCUUCAGAGGCC | |
| R17821 | ACAGCUUAUUUGGAAGCUGAAAUGUGAGGUUUAUAACA | 572 |
| | CUCACAAGAAUCCUGAAAAAGGAUGCCAAACCUCAGGA | |
| | GCUUCAGAGGCCG | |
| R17822 | ACAGCUUAUUUGGAAGCUGAAAUGUGAGGUUUAUAACA | 573 |
| | CUCACAAGAAUCCUGAAAAAGGAUGCCAAACAGGAGCU | |
| | UCAGAGGCCGAGG | |
| R17823 | ACAGCUUAUUUGGAAGCUGAAAUGUGAGGUUUAUAACA | 574 |
| | CUCACAAGAAUCCUGAAAAAGGAUGCCAAACUGAAGCU | |
| | CCUGAGGAAAGAG | |
| R17824 | ACAGCUUAUUUGGAAGCUGAAAUGUGAGGUUUAUAACA | 575 |
| | CUCACAAGAAUCCUGAAAAAGGAUGCCAAACGGCCUCU | |
| | GAAGCUCCUGAGG | |
| R17556 | ACAGCUUAUUUGGAAGCUGAAAUGUGAGGUUUAUAACA | 577 |
| | CUCACAAGAAUCCUGAAAAAGGAUGCCAAACGCCUGCU | |
| | GGGCCACCUGGGA | |
| R17557 | ACAGCUUAUUUGGAAGCUGAAAUGUGAGGUUUAUAACA | 578 |
| | CUCACAAGAAUCCUGAAAAAGGAUGCCAAACCCUGGCC | |
| | UGCUGGGCCACCU | |
| R17558 | ACAGCUUAUUUGGAAGCUGAAAUGUGAGGUUUAUAACA | 579 |
| | CUCACAAGAAUCCUGAAAAAGGAUGCCAAACUACCUGG | |
| | CCUGCUGGGCCAC | |
| R17559 | ACAGCUUAUUUGGAAGCUGAAAUGUGAGGUUUAUAACA | 580 |
| | CUCACAAGAAUCCUGAAAAAGGAUGCCAAACGGGAGGG | |
| | AGGCCAGCGGGUG | |
| R17560 | ACAGCUUAUUUGGAAGCUGAAAUGUGAGGUUUAUAACA | 581 |
| | CUCACAAGAAUCCUGAAAAAGGAUGCCAAACCCCCCAG | |
| | CCCAGUCCCACCA | |
| R17825 | ACAGCUUAUUUGGAAGCUGAAAUGUGAGGUUUAUAACA | 582 |
| | CUCACAAGAAUCCUGAAAAAGGAUGCCAAACCCCUGCU | |
| | CUGUUGCUUCCCC | |
| R15784 | ACAGCUUAUUUGGAAGCUGAAAUGUGAGGUUUAUAACA | 583 |
| | CUCACAAGAAUCCUGAAAAAGGAUGCCAAACGCCUCAG | |
| | GGUUCAAAUCCCA | |
| R15788 | ACAGCUUAUUUGGAAGCUGAAAUGUGAGGUUUAUAACA | 584 |
| | CUCACAAGAAUCCUGAAAAAGGAUGCCAAACGCCCUGC | |
| | AUGAAGCCAAGAA | |
| R16927 | ACAGCUUAUUUGGAAGCUGAAAUGUGAGGUUUAUAACA | 826 |
| | CUCACAAGAAUCCUGAAAAAGGAUGCCAAACAGUUCUG | |
| | GGAUUUGGACCCU | |
| R16928 | ACAGCUUAUUUGGAAGCUGAAAUGUGAGGUUUAUAACA | 827 |
| | CUCACAAGAAUCCUGAAAAAGGAUGCCAAACGACCCUG | |
| | AGGUCAGACCAAC | |
| R16929 | ACAGCUUAUUUGGAAGCUGAAAUGUGAGGUUUAUAACA | 828 |
| | CUCACAAGAAUCCUGAAAAAGGAUGCCAAACACCUCAG | |
| | GGUCCAAAUCCCA | |
| n/a | ACAGCUUAUUUGGAAGCUGAAAUGUGAGGUUUAUAACA | 1570 |
| | CUCACAAGAAUCCUGAAAAAGGAUGCCAAACGACAGCC | |
| | CAGUCCUACCCCA | |
| n/a | ACAGCUUAUUUGGAAGCUGAAAUGUGAGGUUUAUAACA | 1571 |
| | CUCACAAGAAUCCUGAAAAAGGAUGCCAAACUUCAGGG | |
| | CUUGGGGCUGGUG | |
| n/a | ACAGCUUAUUUGGAAGCUGAAAUGUGAGGUUUAUAACA | 1572 |
| | CUCACAAGAAUCCUGAAAAAGGAUGCCAAACCCUUUAC | |
| | UCCAAACACCCCC | |
| n/a | ACAGCUUAUUUGGAAGCUGAAAUGUGAGGUUUAUAACA | 1573 |
| | CUCACAAGAAUCCUGAAAAAGGAUGCCAAACCCCCCCA | |
| | CCCCUCAUCAUAA | |
| n/a | ACAGCUUAUUUGGAAGCUGAAAUGUGAGGUUUAUAACA | 1574 |
| | CUCACAAGAAUCCUGAAAAAGGAUGCCAAACUUCAGUC | |
| | UGGUGGGUUUUCU | |
| n/a | ACAGCUUAUUUGGAAGCUGAAAUGUGAGGUUUAUAACA | 1575 |
| | CUCACAAGAAUCCUGAAAAAGGAUGCCAAACUCACUUG | |
| | CCCAAAGCUACAC | |
| n/a | ACAGCUUAUUUGGAAGCUGAAAUGUGAGGUUUAUAACA | 1576 |
| | CUCACAAGAAUCCUGAAAAAGGAUGCCAAACGUGGGUU | |
| | UUCUGCUCCAUCC | |
| n/a | ACAGCUUAUUUGGAAGCUGAAAUGUGAGGUUUAUAACA | 1577 |
| | CUCACAAGAAUCCUGAAAAAGGAUGCCAAACCCGGAGC | |
| | CACUGAUGCCUGG | |
| n/a | ACAGCUUAUUUGGAAGCUGAAAUGUGAGGUUUAUAACA | 1578 |
| | CUCACAAGAAUCCUGAAAAAGGAUGCCAAACGACUCAG | |
| | UCUCCUAGGGAUU | |
| n/a | ACAGCUUAUUUGGAAGCUGAAAUGUGAGGUUUAUAACA | 1579 |
| | CUCACAAGAAUCCUGAAAAAGGAUGCCAAACGCCUAUG | |
| | UCCAAGCCAUUUC | |
| n/a | ACAGCUUAUUUGGAAGCUGAAAUGUGAGGUUUAUAACA | 1580 |
| | CUCACAAGAAUCCUGAAAAAGGAUGCCAAACCCUCAGG | |
| | CCCUCAUCUCCAC | |
| n/a | ACAGCUUAUUUGGAAGCUGAAAUGUGAGGUUUAUAACA | 1581 |
| | CUCACAAGAAUCCUGAAAAAGGAUGCCAAACUCCAAGC | |
| | CAUUUCCCCUCUC | |
| n/a | ACAGCUUAUUUGGAAGCUGAAAUGUGAGGUUUAUAACA | 1582 |
| | CUCACAAGAAUCCUGAAAAAGGAUGCCAAACAAAGGCU | |
| | GAGAUGGGCCCGA | |
| n/a | ACAGCUUAUUUGGAAGCUGAAAUGUGAGGUUUAUAACA | 1583 |
| | CUCACAAGAAUCCUGAAAAAGGAUGCCAAACAAAUUCC | |
| | AGUGUGAAAGGCU | |
| n/a | ACAGCUUAUUUGGAAGCUGAAAUGUGAGGUUUAUAACA | 1584 |
| | CUCACAAGAAUCCUGAAAAAGGAUGCCAAACGAGAUGA | |
| | UAUAAAACAGGUC | |
| n/a | ACAGCUUAUUUGGAAGCUGAAAUGUGAGGUUUAUAACA | 1585 |
| | CUCACAAGAAUCCUGAAAAAGGAUGCCAAACGGGAGGG | |
| | GAAAGAGGAGGGG | |
| n/a | ACAGCUUAUUUGGAAGCUGAAAUGUGAGGUUUAUAACA | 1586 |
| | CUCACAAGAAUCCUGAAAAAGGAUGCCAAACAAGAACA | |
| | UGGAGGCCCGGGA | |
| n/a | ACAGCUUAUUUGGAAGCUGAAAUGUGAGGUUUAUAACA | 1587 |
| | CUCACAAGAAUCCUGAAAAAGGAUGCCAAACGGGCUGG | |
| | UGGAGGGAGGGGC | |
| n/a | ACAGCUUAUUUGGAAGCUGAAAUGUGAGGUUUAUAACA | 1588 |
| | CUCACAAGAAUCCUGAAAAAGGAUGCCAAACAUGCCUG | |
| | GUCUUCUGUGCCU | |
| n/a | ACAGCUUAUUUGGAAGCUGAAAUGUGAGGUUUAUAACA | 1589 |
| | CUCACAAGAAUCCUGAAAAAGGAUGCCAAACUGUUCAG | |
| | GGCUUGGGGCUGG | |
| n/a | ACAGCUUAUUUGGAAGCUGAAAUGUGAGGUUUAUAACA | 1590 |
| | CUCACAAGAAUCCUGAAAAAGGAUGCCAAACCUCCAGG | |
| | UAAUGCCCUCUGG | |
| n/a | ACAGCUUAUUUGGAAGCUGAAAUGUGAGGUUUAUAACA | 1591 |
| | CUCACAAGAAUCCUGAAAAAGGAUGCCAAACAGGGCUC | |
| | CCCAGGCCCACCC | |
| n/a | ACAGCUUAUUUGGAAGCUGAAAUGUGAGGUUUAUAACA | 1592 |
| | CUCACAAGAAUCCUGAAAAAGGAUGCCAAACUUGGCUG | |
| | GACUGGACGGAGA | |
| n/a | ACAGCUUAUUUGGAAGCUGAAAUGUGAGGUUUAUAACA | 1593 |
| | CUCACAAGAAUCCUGAAAAAGGAUGCCAAACGAGCUAA | |
| | GGAAGCCUCGGAG | |
| n/a | ACAGCUUAUUUGGAAGCUGAAAUGUGAGGUUUAUAACA | 1594 |
| | CUCACAAGAAUCCUGAAAAAGGAUGCCAAACCCCUCUG | |
| | GGGAGGGGAAAGA | |
| n/a | ACAGCUUAUUUGGAAGCUGAAAUGUGAGGUUUAUAACA | 1595 |
| | CUCACAAGAAUCCUGAAAAAGGAUGCCAAACCUCCAAA | |
| | CACCCCCCAGCCC | |
| n/a | ACAGCUUAUUUGGAAGCUGAAAUGUGAGGUUUAUAACA | 1596 |
| | CUCACAAGAAUCCUGAAAAAGGAUGCCAAACGGGAGCC | |
| | AGUCAGCUAGGAA | |
| n/a | ACAGCUUAUUUGGAAGCUGAAAUGUGAGGUUUAUAACA | 1597 |
| | CUCACAAGAAUCCUGAAAAAGGAUGCCAAACGGCCCGA | |
| | GGCCCCUGGCCUA | |
| n/a | ACAGCUUAUUUGGAAGCUGAAAUGUGAGGUUUAUAACA | 1598 |
| | CUCACAAGAAUCCUGAAAAAGGAUGCCAAACGAGGCAG | |
| | CUGCUCCAGGUAA | |
| n/a | ACAGCUUAUUUGGAAGCUGAAAUGUGAGGUUUAUAACA | 1599 |
| | CUCACAAGAAUCCUGAAAAAGGAUGCCAAACGAGGGAG | |
| | GGGCCUGAAAUUC | |
| n/a | ACAGCUUAUUUGGAAGCUGAAAUGUGAGGUUUAUAACA | 1600 |
| | CUCACAAGAAUCCUGAAAAAGGAUGCCAAACGAGAGGG | |
| | CCAGAAAUCACCC | |
| n/a | ACAGCUUAUUUGGAAGCUGAAAUGUGAGGUUUAUAACA | 1601 |
| | CUCACAAGAAUCCUGAAAAAGGAUGCCAAACAAGAAGC | |
| | CCCUCACCCCUCU | |
| n/a | ACAGCUUAUUUGGAAGCUGAAAUGUGAGGUUUAUAACA | 1602 |
| | CUCACAAGAAUCCUGAAAAAGGAUGCCAAACCCCCAGA | |
| | CAGGGAAACUGAG | |
| n/a | ACAGCUUAUUUGGAAGCUGAAAUGUGAGGUUUAUAACA | 1603 |
| | CUCACAAGAAUCCUGAAAAAGGAUGCCAAACGGGCUGG | |
| | AAGUGGCUCCAAG | |
| n/a | ACAGCUUAUUUGGAAGCUGAAAUGUGAGGUUUAUAACA | 1604 |
| | CUCACAAGAAUCCUGAAAAAGGAUGCCAAACCUCCAGG | |
| | CUGUGUUCAGGGC | |
| n/a | ACAGCUUAUUUGGAAGCUGAAAUGUGAGGUUUAUAACA | 1605 |
| | CUCACAAGAAUCCUGAAAAAGGAUGCCAAACGUCUUCU | |
| | GUGCCUUUACUCC | |
| n/a | ACAGCUUAUUUGGAAGCUGAAAUGUGAGGUUUAUAACA | 1606 |
| | CUCACAAGAAUCCUGAAAAAGGAUGCCAAACCACAGGG | |
| | GGUGGGGCUGGAA | |
| n/a | ACAGCUUAUUUGGAAGCUGAAAUGUGAGGUUUAUAACA | 1607 |
| | CUCACAAGAAUCCUGAAAAAGGAUGCCAAACGACUGGA | |
| | CGGAGAUCAGUCC | |
| n/a | ACAGCUUAUUUGGAAGCUGAAAUGUGAGGUUUAUAACA | 1608 |
| | CUCACAAGAAUCCUGAAAAAGGAUGCCAAACGACGGGU | |
| | GCCCCCCACCCCU | |
| n/a | ACAGCUUAUUUGGAAGCUGAAAUGUGAGGUUUAUAACA | 1609 |
| | CUCACAAGAAUCCUGAAAAAGGAUGCCAAACUGUCCAA | |
| | GCCAUUUCCCCUC | |
| n/a | ACAGCUUAUUUGGAAGCUGAAAUGUGAGGUUUAUAACA | 1610 |
| | CUCACAAGAAUCCUGAAAAAGGAUGCCAAACAGAUGGG | |
| | CCCGAGGCCCCUG | |
| n/a | ACAGCUUAUUUGGAAGCUGAAAUGUGAGGUUUAUAACA | 1611 |
| | CUCACAAGAAUCCUGAAAAAGGAUGCCAAACGGUCCUC | |
| | AGUGCCUGCUGCC | |
| n/a | ACAGCUUAUUUGGAAGCUGAAAUGUGAGGUUUAUAACA | 1612 |
| | CUCACAAGAAUCCUGAAAAAGGAUGCCAAACCAGGGCU | |
| | GGCGGGACAGCAG | |
| n/a | ACAGCUUAUUUGGAAGCUGAAAUGUGAGGUUUAUAACA | 1613 |
| | CUCACAAGAAUCCUGAAAAAGGAUGCCAAACCCUUGAU | |
| | GUUCAGUCUGGUG | |
| n/a | ACAGCUUAUUUGGAAGCUGAAAUGUGAGGUUUAUAACA | 1614 |
| | CUCACAAGAAUCCUGAAAAAGGAUGCCAAACAGGCCUG | |
| | GAGAGGGCCAGAA | |
| n/a | ACAGCUUAUUUGGAAGCUGAAAUGUGAGGUUUAUAACA | 1615 |
| | CUCACAAGAAUCCUGAAAAAGGAUGCCAAACCCCUGGA | |
| | GAUGAUAUAAAAC | |
| n/a | ACAGCUUAUUUGGAAGCUGAAAUGUGAGGUUUAUAACA | 1616 |
| | CUCACAAGAAUCCUGAAAAAGGAUGCCAAACACCUGAA | |
| | GAACAUGGAGGCC | |
| n/a | ACAGCUUAUUUGGAAGCUGAAAUGUGAGGUUUAUAACA | 1617 |
| | CUCACAAGAAUCCUGAAAAAGGAUGCCAAACCCUGGUC | |
| | UUCUGUGCCUUUA | |
| n/a | ACAGCUUAUUUGGAAGCUGAAAUGUGAGGUUUAUAACA | 1618 |
| | CUCACAAGAAUCCUGAAAAAGGAUGCCAAACACCUUUG | |
| | CCCAGCGCCCUGG | |
| n/a | ACAGCUUAUUUGGAAGCUGAAAUGUGAGGUUUAUAACA | 1619 |
| | CUCACAAGAAUCCUGAAAAAGGAUGCCAAACCCCAAAG | |
| | CUACACAGGGGGU | |
| n/a | ACAGCUUAUUUGGAAGCUGAAAUGUGAGGUUUAUAACA | 1620 |
| | CUCACAAGAAUCCUGAAAAAGGAUGCCAAACGUGGAGG | |
| | GAGGGGCCUGAAA | |
| n/a | ACAGCUUAUUUGGAAGCUGAAAUGUGAGGUUUAUAACA | 1621 |
| | CUCACAAGAAUCCUGAAAAAGGAUGCCAAACUGCCUUU | |
| | ACUCCAAACACCC | |
| n/a | ACAGCUUAUUUGGAAGCUGAAAUGUGAGGUUUAUAACA | 1622 |
| | CUCACAAGAAUCCUGAAAAAGGAUGCCAAACCUCCAUC | |
| | CCACCCACCUCCC | |
| n/a | ACAGCUUAUUUGGAAGCUGAAAUGUGAGGUUUAUAACA | 1623 |
| | CUCACAAGAAUCCUGAAAAAGGAUGCCAAACAUGUUCA | |
| | GUCUGGUGGGUUU | |
| n/a | ACAGCUUAUUUGGAAGCUGAAAUGUGAGGUUUAUAACA | 1624 |
| | CUCACAAGAAUCCUGAAAAAGGAUGCCAAACCUAGAGC | |
| | UAAGGAAGCCUCG | |
| n/a | ACAGCUUAUUUGGAAGCUGAAAUGUGAGGUUUAUAACA | 1625 |
| | CUCACAAGAAUCCUGAAAAAGGAUGCCAAACGGAAGGA | |
| | AUGAGGGCUCCCC | |
| n/a | ACAGCUUAUUUGGAAGCUGAAAUGUGAGGUUUAUAACA | 1626 |
| | CUCACAAGAAUCCUGAAAAAGGAUGCCAAACGGCUGCA | |
| | GGGCUGGCGGGAC | |
| n/a | ACAGCUUAUUUGGAAGCUGAAAUGUGAGGUUUAUAACA | 1627 |
| | CUCACAAGAAUCCUGAAAAAGGAUGCCAAACAUGCCCU | |
| | CUGGGGAGGGGAA | |
| n/a | ACAGCUUAUUUGGAAGCUGAAAUGUGAGGUUUAUAACA | 1628 |
| | CUCACAAGAAUCCUGAAAAAGGAUGCCAAACAAACAGG | |
| | UCAGAACCCUCCU | |
| n/a | ACAGCUUAUUUGGAAGCUGAAAUGUGAGGUUUAUAACA | 1629 |
| | CUCACAAGAAUCCUGAAAAAGGAUGCCAAACGGGCUGG | |
| | AGGAAGCCUUAGA | |
| n/a | ACAGCUUAUUUGGAAGCUGAAAUGUGAGGUUUAUAACA | 1630 |
| | CUCACAAGAAUCCUGAAAAAGGAUGCCAAACUUCUCAA | |
| | GUCUGAAGAAGCC | |
| n/a | ACAGCUUAUUUGGAAGCUGAAAUGUGAGGUUUAUAACA | 1631 |
| | CUCACAAGAAUCCUGAAAAAGGAUGCCAAACAGCUCAU | |
| | CUGGGCUGCAGGG | |
| n/a | ACAGCUUAUUUGGAAGCUGAAAUGUGAGGUUUAUAACA | 1632 |
| | CUCACAAGAAUCCUGAAAAAGGAUGCCAAACGGGAUUU | |
| | CCCAACUCUCCCG | |
| n/a | ACAGCUUAUUUGGAAGCUGAAAUGUGAGGUUUAUAACA | 1633 |
| | CUCACAAGAAUCCUGAAAAAGGAUGCCAAACGACGGAG | |
| | AUCAGUCCAGACC | |
| n/a | ACAGCUUAUUUGGAAGCUGAAAUGUGAGGUUUAUAACA | 1634 |
| | CUCACAAGAAUCCUGAAAAAGGAUGCCAAACUGAAAGG | |
| | CUGAGAUGGGCCC | |
| n/a | ACAGCUUAUUUGGAAGCUGAAAUGUGAGGUUUAUAACA | 1635 |
| | CUCACAAGAAUCCUGAAAAAGGAUGCCAAACCCUGUCU | |
| | GCUCAGUUCAUCC | |
| n/a | ACAGCUUAUUUGGAAGCUGAAAUGUGAGGUUUAUAACA | 1636 |
| | CUCACAAGAAUCCUGAAAAAGGAUGCCAAACCAGGUUC | |
| | CCCCCUCAUUCUU | |
| n/a | ACAGCUUAUUUGGAAGCUGAAAUGUGAGGUUUAUAACA | 1637 |
| | CUCACAAGAAUCCUGAAAAAGGAUGCCAAACGUCAGCA | |
| | GGUGACCUUUGCC | |
| n/a | ACAGCUUAUUUGGAAGCUGAAAUGUGAGGUUUAUAACA | 1638 |
| | CUCACAAGAAUCCUGAAAAAGGAUGCCAAACGAGGAAG | |
| | CCUUAGACAGCCC | |
| n/a | ACAGCUUAUUUGGAAGCUGAAAUGUGAGGUUUAUAACA | 1639 |
| | CUCACAAGAAUCCUGAAAAAGGAUGCCAAACCUGCAUC | |
| | UGGACACCCUGCC | |
| n/a | ACAGCUUAUUUGGAAGCUGAAAUGUGAGGUUUAUAACA | 1640 |
| | CUCACAAGAAUCCUGAAAAAGGAUGCCAAACCCCAGCG | |
| | CCCUGGGUCCUCA | |
| n/a | ACAGCUUAUUUGGAAGCUGAAAUGUGAGGUUUAUAACA | 1641 |
| | CUCACAAGAAUCCUGAAAAAGGAUGCCAAACCCCAGCC | |
| | CAGCCAGCAAGCC | |
| n/a | ACAGCUUAUUUGGAAGCUGAAAUGUGAGGUUUAUAACA | 1642 |
| | CUCACAAGAAUCCUGAAAAAGGAUGCCAAACGGUUUUC | |
| | UGCUCCAUCCCAC | |
| n/a | ACAGCUUAUUUGGAAGCUGAAAUGUGAGGUUUAUAACA | 1643 |
| | CUCACAAGAAUCCUGAAAAAGGAUGCCAAACUAAAACA | |
| | GGUCAGAACCCUC | |
| n/a | ACAGCUUAUUUGGAAGCUGAAAUGUGAGGUUUAUAACA | 1644 |
| | CUCACAAGAAUCCUGAAAAAGGAUGCCAAACCUCAGUU | |
| | CAUCCCUAGAGGC | |
| n/a | ACAGCUUAUUUGGAAGCUGAAAUGUGAGGUUUAUAACA | 1645 |
| | CUCACAAGAAUCCUGAAAAAGGAUGCCAAACGACACCC | |
| | UGCCUCAGGCCCU | |
| n/a | ACAGCUUAUUUGGAAGCUGAAAUGUGAGGUUUAUAACA | 1646 |
| | CUCACAAGAAUCCUGAAAAAGGAUGCCAAACGCGGGAC | |
| | AGCAGCGUGGACU | |
| n/a | ACAGCUUAUUUGGAAGCUGAAAUGUGAGGUUUAUAACA | 1647 |
| | CUCACAAGAAUCCUGAAAAAGGAUGCCAAACAAGAGGG | |
| | GCAAGAGGAGCUC | |
| n/a | ACAGCUUAUUUGGAAGCUGAAAUGUGAGGUUUAUAACA | 1648 |
| | CUCACAAGAAUCCUGAAAAAGGAUGCCAAACCAUCUGG | |
| | ACACCCUGCCUCA | |
| n/a | ACAGCUUAUUUGGAAGCUGAAAUGUGAGGUUUAUAACA | 1649 |
| | CUCACAAGAAUCCUGAAAAAGGAUGCCAAACGAGGCCC | |
| | GGGAGGGGUGUCA | |
| n/a | ACAGCUUAUUUGGAAGCUGAAAUGUGAGGUUUAUAACA | 1650 |
| | CUCACAAGAAUCCUGAAAAAGGAUGCCAAACCCUGCUG | |
| | CCCUGGAGAUGAU | |
| n/a | ACAGCUUAUUUGGAAGCUGAAAUGUGAGGUUUAUAACA | 1651 |
| | CUCACAAGAAUCCUGAAAAAGGAUGCCAAACAGGAAGC | |
| | CUCGGAGCUGGAC | |
| n/a | ACAGCUUAUUUGGAAGCUGAAAUGUGAGGUUUAUAACA | 1652 |
| | CUCACAAGAAUCCUGAAAAAGGAUGCCAAACUCUGCUC | |
| | AGUUCAUCCCUAG | |
| n/a | ACAGCUUAUUUGGAAGCUGAAAUGUGAGGUUUAUAACA | 1653 |
| | CUCACAAGAAUCCUGAAAAAGGAUGCCAAACGAAGUGG | |
| | CUCCAAGUGCAGG | |
| n/a | ACAGCUUAUUUGGAAGCUGAAAUGUGAGGUUUAUAACA | 1654 |
| | CUCACAAGAAUCCUGAAAAAGGAUGCCAAACGCUGGAC | |
| | UGGACGGAGAUCA | |
| n/a | ACAGCUUAUUUGGAAGCUGAAAUGUGAGGUUUAUAACA | 1655 |
| | CUCACAAGAAUCCUGAAAAAGGAUGCCAAACGAGAAGC | |
| | ACUUGCUAGAGCU | |
| n/a | ACAGCUUAUUUGGAAGCUGAAAUGUGAGGUUUAUAACA | 1656 |
| | CUCACAAGAAUCCUGAAAAAGGAUGCCAAACCUGCCCU | |
| | GGAGAUGAUAUAA | |
| n/a | ACAGCUUAUUUGGAAGCUGAAAUGUGAGGUUUAUAACA | 1657 |
| | CUCACAAGAAUCCUGAAAAAGGAUGCCAAACAUAUAAA | |
| | ACAGGUCAGAACC | |
| n/a | ACAGCUUAUUUGGAAGCUGAAAUGUGAGGUUUAUAACA | 1658 |
| | CUCACAAGAAUCCUGAAAAAGGAUGCCAAACGCUCCAA | |
| | GUGCAGGUUCCCC | |
| n/a | ACAGCUUAUUUGGAAGCUGAAAUGUGAGGUUUAUAACA | 1659 |
| | CUCACAAGAAUCCUGAAAAAGGAUGCCAAACGGCUGGG | |
| | CAGGGAGCUCCUC | |
| n/a | ACAGCUUAUUUGGAAGCUGAAAUGUGAGGUUUAUAACA | 1660 |
| | CUCACAAGAAUCCUGAAAAAGGAUGCCAAACGACAUAG | |
| | GCCAGGGGCCUCG | |
| n/a | ACAGCUUAUUUGGAAGCUGAAAUGUGAGGUUUAUAACA | 1661 |
| | CUCACAAGAAUCCUGAAAAAGGAUGCCAAACGGCCUGG | |
| | GGAGCCCUCAUUC | |
| n/a | ACAGCUUAUUUGGAAGCUGAAAUGUGAGGUUUAUAACA | 1662 |
| | CUCACAAGAAUCCUGAAAAAGGAUGCCAAACGGCUGGG | |
| | GGGUGUUUGGAGU | |
| n/a | ACAGCUUAUUUGGAAGCUGAAAUGUGAGGUUUAUAACA | 1663 |
| | CUCACAAGAAUCCUGAAAAAGGAUGCCAAACGGUGGGA | |
| | UGGAGCAGAAAAC | |
| n/a | ACAGCUUAUUUGGAAGCUGAAAUGUGAGGUUUAUAACA | 1664 |
| | CUCACAAGAAUCCUGAAAAAGGAUGCCAAACGCUUGGA | |
| | CAUAGGCCAGGGG | |
| n/a | ACAGCUUAUUUGGAAGCUGAAAUGUGAGGUUUAUAACA | 1665 |
| | CUCACAAGAAUCCUGAAAAAGGAUGCCAAACAGGGCCU | |
| | GAGGCAGGGUGUC | |
| n/a | ACAGCUUAUUUGGAAGCUGAAAUGUGAGGUUUAUAACA | 1666 |
| | CUCACAAGAAUCCUGAAAAAGGAUGCCAAACAGGCAGG | |
| | GUGUCCAGAUGCA | |
| n/a | ACAGCUUAUUUGGAAGCUGAAAUGUGAGGUUUAUAACA | 1667 |
| | CUCACAAGAAUCCUGAAAAAGGAUGCCAAACUCCCGCC | |
| | AGCCCUGCAGCCC | |
| n/a | ACAGCUUAUUUGGAAGCUGAAAUGUGAGGUUUAUAACA | 1668 |
| | CUCACAAGAAUCCUGAAAAAGGAUGCCAAACCCCCUCU | |
| | UCAUCCUCCUCCC | |
| n/a | ACAGCUUAUUUGGAAGCUGAAAUGUGAGGUUUAUAACA | 1669 |
| | CUCACAAGAAUCCUGAAAAAGGAUGCCAAACAACAUCA | |
| | AGGCACCUGCGGU | |
| n/a | ACAGCUUAUUUGGAAGCUGAAAUGUGAGGUUUAUAACA | 1670 |
| | CUCACAAGAAUCCUGAAAAAGGAUGCCAAACAGCUCAG | |
| | GAACUGGGGGUGG | |
| n/a | ACAGCUUAUUUGGAAGCUGAAAUGUGAGGUUUAUAACA | 1671 |
| | CUCACAAGAAUCCUGAAAAAGGAUGCCAAACUUCUUCA | |
| | GGUUAUGAUGAGG | |
| n/a | ACAGCUUAUUUGGAAGCUGAAAUGUGAGGUUUAUAACA | 1672 |
| | CUCACAAGAAUCCUGAAAAAGGAUGCCAAACGCUUUGG | |
| | GCAAGUGACACCC | |
| n/a | ACAGCUUAUUUGGAAGCUGAAAUGUGAGGUUUAUAACA | 1673 |
| | CUCACAAGAAUCCUGAAAAAGGAUGCCAAACAGGGGGG | |
| | AACCUGCACUUGG | |
| n/a | ACAGCUUAUUUGGAAGCUGAAAUGUGAGGUUUAUAACA | 1674 |
| | CUCACAAGAAUCCUGAAAAAGGAUGCCAAACGCUUGGG | |
| | CUGGGGGGUGUUU | |
| n/a | ACAGCUUAUUUGGAAGCUGAAAUGUGAGGUUUAUAACA | 1675 |
| | CUCACAAGAAUCCUGAAAAAGGAUGCCAAACAACUGAG | |
| | CAGACAGGCAGGA | |
| n/a | ACAGCUUAUUUGGAAGCUGAAAUGUGAGGUUUAUAACA | 1676 |
| | CUCACAAGAAUCCUGAAAAAGGAUGCCAAACUGUGUCU | |
| | UUGGGUGAUUUCU | |
| n/a | ACAGCUUAUUUGGAAGCUGAAAUGUGAGGUUUAUAACA | 1677 |
| | CUCACAAGAAUCCUGAAAAAGGAUGCCAAACUGAUGAG | |
| | GGGUGGGGGGCAC | |
| n/a | ACAGCUUAUUUGGAAGCUGAAAUGUGAGGUUUAUAACA | 1678 |
| | CUCACAAGAAUCCUGAAAAAGGAUGCCAAACGGCAGGG | |
| | AGCUCCUCUUGCC | |
| n/a | ACAGCUUAUUUGGAAGCUGAAAUGUGAGGUUUAUAACA | 1679 |
| | CUCACAAGAAUCCUGAAAAAGGAUGCCAAACAGGGGUG | |
| | GGGGGCACCCGUC | |
| n/a | ACAGCUUAUUUGGAAGCUGAAAUGUGAGGUUUAUAACA | 1680 |
| | CUCACAAGAAUCCUGAAAAAGGAUGCCAAACCCUGGAG | |
| | CAGCUGCCUCUAG | |
| n/a | ACAGCUUAUUUGGAAGCUGAAAUGUGAGGUUUAUAACA | 1681 |
| | CUCACAAGAAUCCUGAAAAAGGAUGCCAAACGGGAUGA | |
| | ACUGAGCAGACAG | |
| n/a | ACAGCUUAUUUGGAAGCUGAAAUGUGAGGUUUAUAACA | 1682 |
| | CUCACAAGAAUCCUGAAAAAGGAUGCCAAACAGGCUUC | |
| | CUCCAGCCCUAAG | |
| n/a | ACAGCUUAUUUGGAAGCUGAAAUGUGAGGUUUAUAACA | 1683 |
| | CUCACAAGAAUCCUGAAAAAGGAUGCCAAACGAGAUGA | |
| | GGGCCUGAGGCAG | |
| n/a | ACAGCUUAUUUGGAAGCUGAAAUGUGAGGUUUAUAACA | 1684 |
| | CUCACAAGAAUCCUGAAAAAGGAUGCCAAACGGAUGGA | |
| | GCAGAAAACCCAC | |
| n/a | ACAGCUUAUUUGGAAGCUGAAAUGUGAGGUUUAUAACA | 1685 |
| | CUCACAAGAAUCCUGAAAAAGGAUGCCAAACAAGAAUG | |
| | AGGGGGGAACCUG | |
| n/a | ACAGCUUAUUUGGAAGCUGAAAUGUGAGGUUUAUAACA | 1686 |
| | CUCACAAGAAUCCUGAAAAAGGAUGCCAAACUUUGGAG | |
| | UAAAGGCACAGAA | |
| n/a | ACAGCUUAUUUGGAAGCUGAAAUGUGAGGUUUAUAACA | 1687 |
| | CUCACAAGAAUCCUGAAAAAGGAUGCCAAACGAGUAGA | |
| | GGGGUGAGGGGCU | |
| n/a | ACAGCUUAUUUGGAAGCUGAAAUGUGAGGUUUAUAACA | 1688 |
| | CUCACAAGAAUCCUGAAAAAGGAUGCCAAACACCUGUU | |
| | UUAUAUCAUCUCC | |
| n/a | ACAGCUUAUUUGGAAGCUGAAAUGUGAGGUUUAUAACA | 1689 |
| | CUCACAAGAAUCCUGAAAAAGGAUGCCAAACGUGAGAG | |
| | GGGAAAUGGCUUG | |
| n/a | ACAGCUUAUUUGGAAGCUGAAAUGUGAGGUUUAUAACA | 1690 |
| | CUCACAAGAAUCCUGAAAAAGGAUGCCAAACUGUAGCU | |
| | UUGGGCAAGUGAC | |
| n/a | ACAGCUUAUUUGGAAGCUGAAAUGUGAGGUUUAUAACA | 1691 |
| | CUCACAAGAAUCCUGAAAAAGGAUGCCAAACUGUCUUU | |
| | GGGUGAUUUCUGG | |
| n/a | ACAGCUUAUUUGGAAGCUGAAAUGUGAGGUUUAUAACA | 1692 |
| | CUCACAAGAAUCCUGAAAAAGGAUGCCAAACUAUCAUC | |
| | UCCAGGGCAGCAG | |
| n/a | ACAGCUUAUUUGGAAGCUGAAAUGUGAGGUUUAUAACA | 1693 |
| | CUCACAAGAAUCCUGAAAAAGGAUGCCAAACGAGCAGA | |
| | AAACCCACCAGAC | |
| n/a | ACAGCUUAUUUGGAAGCUGAAAUGUGAGGUUUAUAACA | 1694 |
| | CUCACAAGAAUCCUGAAAAAGGAUGCCAAACGGAGACU | |
| | GAGUCCACGCUGC | |
| n/a | ACAGCUUAUUUGGAAGCUGAAAUGUGAGGUUUAUAACA | 1695 |
| | CUCACAAGAAUCCUGAAAAAGGAUGCCAAACCAGCCCA | |
| | GAUGAGCUCAGGA | |
| n/a | ACAGCUUAUUUGGAAGCUGAAAUGUGAGGUUUAUAACA | 1696 |
| | CUCACAAGAAUCCUGAAAAAGGAUGCCAAACGGUGGCU | |
| | UGGGCUGGGGGGU | |
| n/a | ACAGCUUAUUUGGAAGCUGAAAUGUGAGGUUUAUAACA | 1697 |
| | CUCACAAGAAUCCUGAAAAAGGAUGCCAAACAGGGGCU | |
| | UCUUCAGACUUGA | |
| n/a | ACAGCUUAUUUGGAAGCUGAAAUGUGAGGUUUAUAACA | 1698 |
| | CUCACAAGAAUCCUGAAAAAGGAUGCCAAACCACUUGG | |
| | AGCCACUUCCAGC | |
| n/a | ACAGCUUAUUUGGAAGCUGAAAUGUGAGGUUUAUAACA | 1699 |
| | CUCACAAGAAUCCUGAAAAAGGAUGCCAAACCAGCAAG | |
| | CGGGCGGGAGAGU | |
| n/a | ACAGCUUAUUUGGAAGCUGAAAUGUGAGGUUUAUAACA | 1700 |
| | CUCACAAGAAUCCUGAAAAAGGAUGCCAAACGGCAAAG | |
| | GUCACCUGCUGAC | |
| n/a | ACAGCUUAUUUGGAAGCUGAAAUGUGAGGUUUAUAACA | 1701 |
| | CUCACAAGAAUCCUGAAAAAGGAUGCCAAACUCUUUGG | |
| | GUGAUUUCUGGCC | |
| n/a | ACAGCUUAUUUGGAAGCUGAAAUGUGAGGUUUAUAACA | 1702 |
| | CUCACAAGAAUCCUGAAAAAGGAUGCCAAACUCAUCUC | |
| | CAGGGCAGCAGGC | |
| n/a | ACAGCUUAUUUGGAAGCUGAAAUGUGAGGUUUAUAACA | 1703 |
| | CUCACAAGAAUCCUGAAAAAGGAUGCCAAACAGCAGAC | |
| | AGGCAGGAGGGUU | |
| n/a | ACAGCUUAUUUGGAAGCUGAAAUGUGAGGUUUAUAACA | 1704 |
| | CUCACAAGAAUCCUGAAAAAGGAUGCCAAACAGGACCC | |
| | AGGGCGCUGGGCA | |
| n/a | ACAGCUUAUUUGGAAGCUGAAAUGUGAGGUUUAUAACA | 1705 |
| | CUCACAAGAAUCCUGAAAAAGGAUGCCAAACGGGGGUG | |
| | UUUGGAGUAAAGG | |
| n/a | ACAGCUUAUUUGGAAGCUGAAAUGUGAGGUUUAUAACA | 1706 |
| | CUCACAAGAAUCCUGAAAAAGGAUGCCAAACGGGUAGG | |
| | ACUGGGCUGUCUA | |
| n/a | ACAGCUUAUUUGGAAGCUGAAAUGUGAGGUUUAUAACA | 1707 |
| | CUCACAAGAAUCCUGAAAAAGGAUGCCAAACGGUGAUU | |
| | UCUGGCCCUCUCC | |
| n/a | ACAGCUUAUUUGGAAGCUGAAAUGUGAGGUUUAUAACA | 1708 |
| | CUCACAAGAAUCCUGAAAAAGGAUGCCAAACGAGCAGC | |
| | UGCCUCUAGGGAU | |
| n/a | ACAGCUUAUUUGGAAGCUGAAAUGUGAGGUUUAUAACA | 1709 |
| | CUCACAAGAAUCCUGAAAAAGGAUGCCAAACCUGUGUG | |
| | UCUUUGGGUGAUU | |
| n/a | ACAGCUUAUUUGGAAGCUGAAAUGUGAGGUUUAUAACA | 1710 |
| | CUCACAAGAAUCCUGAAAAAGGAUGCCAAACCUGACCA | |
| | GUGGAGAUGAGGG | |
| n/a | ACAGCUUAUUUGGAAGCUGAAAUGUGAGGUUUAUAACA | 1711 |
| | CUCACAAGAAUCCUGAAAAAGGAUGCCAAACAUGAGGG | |
| | GUGGGGGGCACCC | |
| n/a | ACAGCUUAUUUGGAAGCUGAAAUGUGAGGUUUAUAACA | 1712 |
| | CUCACAAGAAUCCUGAAAAAGGAUGCCAAACUCUAAGG | |
| | CUUCCUCCAGCCC | |
| n/a | ACAGCUUAUUUGGAAGCUGAAAUGUGAGGUUUAUAACA | 1713 |
| | CUCACAAGAAUCCUGAAAAAGGAUGCCAAACGAAUUUC | |
| | AGGCCCCUCCCUC | |
| n/a | ACAGCUUAUUUGGAAGCUGAAAUGUGAGGUUUAUAACA | 1714 |
| | CUCACAAGAAUCCUGAAAAAGGAUGCCAAACAGCCUGA | |
| | AGAAUGAGGGGGG | |
| n/a | ACAGCUUAUUUGGAAGCUGAAAUGUGAGGUUUAUAACA | 1715 |
| | CUCACAAGAAUCCUGAAAAAGGAUGCCAAACGGGGGCA | |
| | CCCGUCCAGCUCC | |
| n/a | ACAGCUUAUUUGGAAGCUGAAAUGUGAGGUUUAUAACA | 1716 |
| | CUCACAAGAAUCCUGAAAAAGGAUGCCAAACAAGGCAC | |
| | AGAAGACCAGGCA | |
| n/a | ACAGCUUAUUUGGAAGCUGAAAUGUGAGGUUUAUAACA | 1717 |
| | CUCACAAGAAUCCUGAAAAAGGAUGCCAAACACCAGUG | |
| | GAGAUGAGGGCCU | |
| n/a | ACAGCUUAUUUGGAAGCUGAAAUGUGAGGUUUAUAACA | 1718 |
| | CUCACAAGAAUCCUGAAAAAGGAUGCCAAACGGCUGUC | |
| | UAAGGCUUCCUCC | |
| n/a | ACAGCUUAUUUGGAAGCUGAAAUGUGAGGUUUAUAACA | 1719 |
| | CUCACAAGAAUCCUGAAAAAGGAUGCCAAACGGGGUGG | |
| | GCCUGGGGAGCCC | |
| n/a | ACAGCUUAUUUGGAAGCUGAAAUGUGAGGUUUAUAACA | 1720 |
| | CUCACAAGAAUCCUGAAAAAGGAUGCCAAACUAGCUUU | |
| | GGGCAAGUGACAC | |
| n/a | ACAGCUUAUUUGGAAGCUGAAAUGUGAGGUUUAUAACA | 1721 |
| | CUCACAAGAAUCCUGAAAAAGGAUGCCAAACGAGCCAC | |
| | UUCCAGCCCCACC | |
| n/a | ACAGCUUAUUUGGAAGCUGAAAUGUGAGGUUUAUAACA | 1722 |
| | CUCACAAGAAUCCUGAAAAAGGAUGCCAAACAUUUCUG | |
| | GCCCUCUCCAGGC | |
| n/a | ACAGCUUAUUUGGAAGCUGAAAUGUGAGGUUUAUAACA | 1723 |
| | CUCACAAGAAUCCUGAAAAAGGAUGCCAAACACUGGCU | |
| | CCCCAGGGAGAGG | |
| n/a | ACAGCUUAUUUGGAAGCUGAAAUGUGAGGUUUAUAACA | 1724 |
| | CUCACAAGAAUCCUGAAAAAGGAUGCCAAACGCCCUCU | |
| | CCAGGCCUCAGUU | |
| n/a | ACAGCUUAUUUGGAAGCUGAAAUGUGAGGUUUAUAACA | 1725 |
| | CUCACAAGAAUCCUGAAAAAGGAUGCCAAACGGACUGG | |
| | GCUGUCUAAGGCU | |
| n/a | ACAGCUUAUUUGGAAGCUGAAAUGUGAGGUUUAUAACA | 1726 |
| | CUCACAAGAAUCCUGAAAAAGGAUGCCAAACCUGUCCC | |
| | GCCAGCCCUGCAG | |
| n/a | ACAGCUUAUUUGGAAGCUGAAAUGUGAGGUUUAUAACA | 1727 |
| | CUCACAAGAAUCCUGAAAAAGGAUGCCAAACGGCAAGU | |
| | GACACCCCUCCCG | |
| n/a | ACAGCUUAUUUGGAAGCUGAAAUGUGAGGUUUAUAACA | 1728 |
| | CUCACAAGAAUCCUGAAAAAGGAUGCCAAACAGAACAA | |
| | GUGGGUGGCUUGG | |
| n/a | ACAGCUUAUUUGGAAGCUGAAAUGUGAGGUUUAUAACA | 1729 |
| | CUCACAAGAAUCCUGAAAAAGGAUGCCAAACAACACAG | |
| | CCUGGAGUAGAGG | |
| n/a | ACAGCUUAUUUGGAAGCUGAAAUGUGAGGUUUAUAACA | 1730 |
| | CUCACAAGAAUCCUGAAAAAGGAUGCCAAACUCUGGGG | |
| | UAGGACUGGGCUG | |
| n/a | ACAGCUUAUUUGGAAGCUGAAAUGUGAGGUUUAUAACA | 1731 |
| | CUCACAAGAAUCCUGAAAAAGGAUGCCAAACGAGGGGU | |
| | GAGGGGCUUCUUC | |
| n/a | ACAGCUUAUUUGGAAGCUGAAAUGUGAGGUUUAUAACA | 1732 |
| | CUCACAAGAAUCCUGAAAAAGGAUGCCAAACCGGUCUG | |
| | GACUGAUCUCCGU | |
| n/a | ACAGCUUAUUUGGAAGCUGAAAUGUGAGGUUUAUAACA | 1733 |
| | CUCACAAGAAUCCUGAAAAAGGAUGCCAAACGCUGGGC | |
| | UGGGCAGGGAGCU | |
| n/a | ACAGCUUAUUUGGAAGCUGAAAUGUGAGGUUUAUAACA | 1734 |
| | CUCACAAGAAUCCUGAAAAAGGAUGCCAAACGGGAGCC | |
| | CUCAUUCCUUCCU | |
| n/a | ACAGCUUAUUUGGAAGCUGAAAUGUGAGGUUUAUAACA | 1735 |
| | CUCACAAGAAUCCUGAAAAAGGAUGCCAAACGAGUAAA | |
| | GGCACAGAAGACC | |
| n/a | ACAGCUUAUUUGGAAGCUGAAAUGUGAGGUUUAUAACA | 1736 |
| | CUCACAAGAAUCCUGAAAAAGGAUGCCAAACGCAAGUG | |
| | CUUCUCCAGGCUU | |
| n/a | ACAGCUUAUUUGGAAGCUGAAAUGUGAGGUUUAUAACA | 1737 |
| | CUCACAAGAAUCCUGAAAAAGGAUGCCAAACAGAGGGG | |
| | AAAUGGCUUGGAC | |
| n/a | ACAGCUUAUUUGGAAGCUGAAAUGUGAGGUUUAUAACA | 1738 |
| | CUCACAAGAAUCCUGAAAAAGGAUGCCAAACAGUCCAC | |
| | GCUGCUGUCCCGC | |
| n/a | ACAGCUUAUUUGGAAGCUGAAAUGUGAGGUUUAUAACA | 1739 |
| | CUCACAAGAAUCCUGAAAAAGGAUGCCAAACCCUCUAG | |
| | GGAUGAACUGAGC | |
| n/a | ACAGCUUAUUUGGAAGCUGAAAUGUGAGGUUUAUAACA | 1740 |
| | CUCACAAGAAUCCUGAAAAAGGAUGCCAAACGGCCAGG | |
| | GGCCUCGGGCCCA | |
| n/a | ACAGCUUAUUUGGAAGCUGAAAUGUGAGGUUUAUAACA | 1741 |
| | CUCACAAGAAUCCUGAAAAAGGAUGCCAAACCUGGCUG | |
| | GGCUGGGCAGGGA | |
| n/a | ACAGCUUAUUUGGAAGCUGAAAUGUGAGGUUUAUAACA | 1742 |
| | CUCACAAGAAUCCUGAAAAAGGAUGCCAAACAUCUCCG | |
| | UCCAGUCCAGCCA | |
| n/a | ACAGCUUAUUUGGAAGCUGAAAUGUGAGGUUUAUAACA | 1743 |
| | CUCACAAGAAUCCUGAAAAAGGAUGCCAAACGACUGAU | |
| | CUCCGUCCAGUCC | |
| n/a | ACAGCUUAUUUGGAAGCUGAAAUGUGAGGUUUAUAACA | 1744 |
| | CUCACAAGAAUCCUGAAAAAGGAUGCCAAACGGAAAUC | |
| | CCUAGGAGACUGA | |
| n/a | ACAGCUUAUUUGGAAGCUGAAAUGUGAGGUUUAUAACA | 1745 |
| | CUCACAAGAAUCCUGAAAAAGGAUGCCAAACGCUGACU | |
| | GGCUCCCCAGGGA | |
| n/a | ACAGCUUAUUUGGAAGCUGAAAUGUGAGGUUUAUAACA | 1746 |
| | CUCACAAGAAUCCUGAAAAAGGAUGCCAAACACACCCC | |
| | UCCCGGGCCUCCA | |
| n/a | ACAGCUUAUUUGGAAGCUGAAAUGUGAGGUUUAUAACA | 1747 |
| | CUCACAAGAAUCCUGAAAAAGGAUGCCAAACGCUCUAG | |
| | CAAGUGCUUCUCC | |
| n/a | ACAGCUUAUUUGGAAGCUGAAAUGUGAGGUUUAUAACA | 1748 |
| | CUCACAAGAAUCCUGAAAAAGGAUGCCAAACCUUCUCC | |
| | AGGCUUGCUGGCU | |
| n/a | ACAGCUUAUUUGGAAGCUGAAAUGUGAGGUUUAUAACA | 1749 |
| | CUCACAAGAAUCCUGAAAAAGGAUGCCAAACUUUUAUA | |
| | UCAUCUCCAGGGC | |
| n/a | ACAGCUUAUUUGGAAGCUGAAAUGUGAGGUUUAUAACA | 1750 |
| | CUCACAAGAAUCCUGAAAAAGGAUGCCAAACUCCAGAU | |
| | GCAGCAAGCGGGC | |
| n/a | ACAGCUUAUUUGGAAGCUGAAAUGUGAGGUUUAUAACA | 1751 |
| | CUCACAAGAAUCCUGAAAAAGGAUGCCAAACGCUCCCC | |
| | AGGGAGAGGCUGG | |
| n/a | ACAGCUUAUUUGGAAGCUGAAAUGUGAGGUUUAUAACA | 1752 |
| | CUCACAAGAAUCCUGAAAAAGGAUGCCAAACACAGGCU | |
| | CCUUCACAGAAGC | |
| n/a | ACAGCUUAUUUGGAAGCUGAAAUGUGAGGUUUAUAACA | 1753 |
| | CUCACAAGAAUCCUGAAAAAGGAUGCCAAACCUCUGCA | |
| | GAACGGGACAUCU | |
| n/a | ACAGCUUAUUUGGAAGCUGAAAUGUGAGGUUUAUAACA | 1754 |
| | CUCACAAGAAUCCUGAAAAAGGAUGCCAAACGCCCCCC | |
| | CCACACCAGGAAC | |
| n/a | ACAGCUUAUUUGGAAGCUGAAAUGUGAGGUUUAUAACA | 1755 |
| | CUCACAAGAAUCCUGAAAAAGGAUGCCAAACAUAUGCU | |
| | GAGAAACAAUAGG | |
| n/a | ACAGCUUAUUUGGAAGCUGAAAUGUGAGGUUUAUAACA | 1756 |
| | CUCACAAGAAUCCUGAAAAAGGAUGCCAAACGGCAGGG | |
| | AUUAGUGAGAGAA | |
| n/a | ACAGCUUAUUUGGAAGCUGAAAUGUGAGGUUUAUAACA | 1757 |
| | CUCACAAGAAUCCUGAAAAAGGAUGCCAAACGACCUCA | |
| | GGCCUGCUUUACA | |
| n/a | ACAGCUUAUUUGGAAGCUGAAAUGUGAGGUUUAUAACA | 1758 |
| | CUCACAAGAAUCCUGAAAAAGGAUGCCAAACCAGAACG | |
| | GGACAUCUACUCU | |
| n/a | ACAGCUUAUUUGGAAGCUGAAAUGUGAGGUUUAUAACA | 1759 |
| | CUCACAAGAAUCCUGAAAAAGGAUGCCAAACGAGUAGC | |
| | UAGCUGCUUCUAG | |
| n/a | ACAGCUUAUUUGGAAGCUGAAAUGUGAGGUUUAUAACA | 1760 |
| | CUCACAAGAAUCCUGAAAAAGGAUGCCAAACGUGCCAC | |
| | UGUGGAGCAACGG | |
| n/a | ACAGCUUAUUUGGAAGCUGAAAUGUGAGGUUUAUAACA | 1761 |
| | CUCACAAGAAUCCUGAAAAAGGAUGCCAAACGGGAAUC | |
| | UGUGGUGCCACUG | |
| n/a | ACAGCUUAUUUGGAAGCUGAAAUGUGAGGUUUAUAACA | 1762 |
| | CUCACAAGAAUCCUGAAAAAGGAUGCCAAACGUGAGAG | |
| | CUUCUCCCUCCAG | |
| n/a | ACAGCUUAUUUGGAAGCUGAAAUGUGAGGUUUAUAACA | 1763 |
| | CUCACAAGAAUCCUGAAAAAGGAUGCCAAACGCUCCAG | |
| | GAUGGGACAGCGG | |
| n/a | ACAGCUUAUUUGGAAGCUGAAAUGUGAGGUUUAUAACA | 1764 |
| | CUCACAAGAAUCCUGAAAAAGGAUGCCAAACCUGAGAA | |
| | ACAAUAGGUUUCU | |
| n/a | ACAGCUUAUUUGGAAGCUGAAAUGUGAGGUUUAUAACA | 1765 |
| | CUCACAAGAAUCCUGAAAAAGGAUGCCAAACACCUGUU | |
| | UUAUAUUGGCUCC | |
| n/a | ACAGCUUAUUUGGAAGCUGAAAUGUGAGGUUUAUAACA | 1766 |
| | CUCACAAGAAUCCUGAAAAAGGAUGCCAAACAGAUUGC | |
| | CCAUGCUUUUCAC | |
| n/a | ACAGCUUAUUUGGAAGCUGAAAUGUGAGGUUUAUAACA | 1767 |
| | CUCACAAGAAUCCUGAAAAAGGAUGCCAAACGGGUGGA | |
| | AGGUGGAGUAUAG | |
| n/a | ACAGCUUAUUUGGAAGCUGAAAUGUGAGGUUUAUAACA | 1768 |
| | CUCACAAGAAUCCUGAAAAAGGAUGCCAAACCCAAAGG | |
| | CUUCUAAUAGCUC | |
| n/a | ACAGCUUAUUUGGAAGCUGAAAUGUGAGGUUUAUAACA | 1769 |
| | CUCACAAGAAUCCUGAAAAAGGAUGCCAAACAGACAGG | |
| | AAAAGACAGGGGA | |
| n/a | ACAGCUUAUUUGGAAGCUGAAAUGUGAGGUUUAUAACA | 1770 |
| | CUCACAAGAAUCCUGAAAAAGGAUGCCAAACGACCCAG | |
| | CCCCCCAAGUGGG | |
| n/a | ACAGCUUAUUUGGAAGCUGAAAUGUGAGGUUUAUAACA | 1771 |
| | CUCACAAGAAUCCUGAAAAAGGAUGCCAAACCCGUAGC | |
| | UGGGCAGGGAUUA | |
| n/a | ACAGCUUAUUUGGAAGCUGAAAUGUGAGGUUUAUAACA | 1772 |
| | CUCACAAGAAUCCUGAAAAAGGAUGCCAAACCAGUAGA | |
| | CCCAGCCCCCCAA | |
| n/a | ACAGCUUAUUUGGAAGCUGAAAUGUGAGGUUUAUAACA | 1773 |
| | CUCACAAGAAUCCUGAAAAAGGAUGCCAAACGGUGGUG | |
| | AGAGCUUCUCCCU | |
| n/a | ACAGCUUAUUUGGAAGCUGAAAUGUGAGGUUUAUAACA | 1774 |
| | CUCACAAGAAUCCUGAAAAAGGAUGCCAAACGAAUGGA | |
| | AUGGGGAAUCUGU | |
| n/a | ACAGCUUAUUUGGAAGCUGAAAUGUGAGGUUUAUAACA | 1775 |
| | CUCACAAGAAUCCUGAAAAAGGAUGCCAAACGAUGGCU | |
| | GGGUGGUGAGAGC | |
| n/a | ACAGCUUAUUUGGAAGCUGAAAUGUGAGGUUUAUAACA | 1776 |
| | CUCACAAGAAUCCUGAAAAAGGAUGCCAAACUGGAGCA | |
| | ACGGAGGAAGUGG | |
| n/a | ACAGCUUAUUUGGAAGCUGAAAUGUGAGGUUUAUAACA | 1777 |
| | CUCACAAGAAUCCUGAAAAAGGAUGCCAAACGCCUCCC | |
| | UUUCCCCAGCUUC | |
| n/a | ACAGCUUAUUUGGAAGCUGAAAUGUGAGGUUUAUAACA | 1778 |
| | CUCACAAGAAUCCUGAAAAAGGAUGCCAAACGGUCAUG | |
| | AAUCCCAAGCCUU | |
| n/a | ACAGCUUAUUUGGAAGCUGAAAUGUGAGGUUUAUAACA | 1779 |
| | CUCACAAGAAUCCUGAAAAAGGAUGCCAAACGCUAGCU | |
| | GCUUCUAGGGAUA | |
| n/a | ACAGCUUAUUUGGAAGCUGAAAUGUGAGGUUUAUAACA | 1780 |
| | CUCACAAGAAUCCUGAAAAAGGAUGCCAAACGCCCAUA | |
| | GCCUCCCUUUCCC | |
| n/a | ACAGCUUAUUUGGAAGCUGAAAUGUGAGGUUUAUAACA | 1781 |
| | CUCACAAGAAUCCUGAAAAAGGAUGCCAAACGAGACCA | |
| | UGAGUCCCAAGCC | |
| n/a | ACAGCUUAUUUGGAAGCUGAAAUGUGAGGUUUAUAACA | 1782 |
| | CUCACAAGAAUCCUGAAAAAGGAUGCCAAACUGACCUG | |
| | UUUUAUAUUGGCU | |
| n/a | ACAGCUUAUUUGGAAGCUGAAAUGUGAGGUUUAUAACA | 1783 |
| | CUCACAAGAAUCCUGAAAAAGGAUGCCAAACCUCUAAU | |
| | AUGCUGAGAAACA | |
| n/a | ACAGCUUAUUUGGAAGCUGAAAUGUGAGGUUUAUAACA | 1784 |
| | CUCACAAGAAUCCUGAAAAAGGAUGCCAAACCCACUGU | |
| | GGAGCAACGGAGG | |
| n/a | ACAGCUUAUUUGGAAGCUGAAAUGUGAGGUUUAUAACA | 1785 |
| | CUCACAAGAAUCCUGAAAAAGGAUGCCAAACACCUCCA | |
| | CCUGUGAUCCCAA | |
| n/a | ACAGCUUAUUUGGAAGCUGAAAUGUGAGGUUUAUAACA | 1786 |
| | CUCACAAGAAUCCUGAAAAAGGAUGCCAAACCUUUACA | |
| | GCCUAGAGCCAGU | |
| n/a | ACAGCUUAUUUGGAAGCUGAAAUGUGAGGUUUAUAACA | 1787 |
| | CUCACAAGAAUCCUGAAAAAGGAUGCCAAACCUGAGCA | |
| | GUCCAGACCAGAG | |
| n/a | ACAGCUUAUUUGGAAGCUGAAAUGUGAGGUUUAUAACA | 1788 |
| | CUCACAAGAAUCCUGAAAAAGGAUGCCAAACAGGCAGG | |
| | AAGGCCAUGCAGC | |
| n/a | ACAGCUUAUUUGGAAGCUGAAAUGUGAGGUUUAUAACA | 1789 |
| | CUCACAAGAAUCCUGAAAAAGGAUGCCAAACUUGGCUC | |
| | CAGGAUGGGACAG | |
| n/a | ACAGCUUAUUUGGAAGCUGAAAUGUGAGGUUUAUAACA | 1790 |
| | CUCACAAGAAUCCUGAAAAAGGAUGCCAAACAAAGCCU | |
| | UCCAGUAGAAUGG | |
| n/a | ACAGCUUAUUUGGAAGCUGAAAUGUGAGGUUUAUAACA | 1791 |
| | CUCACAAGAAUCCUGAAAAAGGAUGCCAAACAUAUUAG | |
| | AUAGAGAACUACA | |
| n/a | ACAGCUUAUUUGGAAGCUGAAAUGUGAGGUUUAUAACA | 1792 |
| | CUCACAAGAAUCCUGAAAAAGGAUGCCAAACCCCAGUG | |
| | CAAGGCUUUUGGC | |
| n/a | ACAGCUUAUUUGGAAGCUGAAAUGUGAGGUUUAUAACA | 1793 |
| | CUCACAAGAAUCCUGAAAAAGGAUGCCAAACUAGAAAU | |
| | ACUCUGCAGAACG | |
| n/a | ACAGCUUAUUUGGAAGCUGAAAUGUGAGGUUUAUAACA | 1794 |
| | CUCACAAGAAUCCUGAAAAAGGAUGCCAAACAAUCCCA | |
| | AGCCUUUCUCCCA | |
| n/a | ACAGCUUAUUUGGAAGCUGAAAUGUGAGGUUUAUAACA | 1795 |
| | CUCACAAGAAUCCUGAAAAAGGAUGCCAAACGAUACCA | |
| | AAGGCUUCUAAUA | |
| n/a | ACAGCUUAUUUGGAAGCUGAAAUGUGAGGUUUAUAACA | 1796 |
| | CUCACAAGAAUCCUGAAAAAGGAUGCCAAACAAACUGA | |
| | GCAGGCAAGCGGG | |
| n/a | ACAGCUUAUUUGGAAGCUGAAAUGUGAGGUUUAUAACA | 1797 |
| | CUCACAAGAAUCCUGAAAAAGGAUGCCAAACCUUUUCA | |
| | CGGCCACCUCCGC | |
| n/a | ACAGCUUAUUUGGAAGCUGAAAUGUGAGGUUUAUAACA | 1798 |
| | CUCACAAGAAUCCUGAAAAAGGAUGCCAAACAGAAAUC | |
| | CCUCUGAGAUUGC | |
| n/a | ACAGCUUAUUUGGAAGCUGAAAUGUGAGGUUUAUAACA | 1799 |
| | CUCACAAGAAUCCUGAAAAAGGAUGCCAAACGAGUAUA | |
| | GAAAUACUCUGCA | |
| n/a | ACAGCUUAUUUGGAAGCUGAAAUGUGAGGUUUAUAACA | 1800 |
| | CUCACAAGAAUCCUGAAAAAGGAUGCCAAACAAGGUUA | |
| | CAUGCCCCCACAC | |
| n/a | ACAGCUUAUUUGGAAGCUGAAAUGUGAGGUUUAUAACA | 1801 |
| | CUCACAAGAAUCCUGAAAAAGGAUGCCAAACUUUCUUC | |
| | CCUUCCUUCCUCU | |
| n/a | ACAGCUUAUUUGGAAGCUGAAAUGUGAGGUUUAUAACA | 1802 |
| | CUCACAAGAAUCCUGAAAAAGGAUGCCAAACUUUUAUA | |
| | UUGGCUCCAGGAU | |
| n/a | ACAGCUUAUUUGGAAGCUGAAAUGUGAGGUUUAUAACA | 1803 |
| | CUCACAAGAAUCCUGAAAAAGGAUGCCAAACGAGCCAG | |
| | UGACAGGCUCCUU | |
| n/a | ACAGCUUAUUUGGAAGCUGAAAUGUGAGGUUUAUAACA | 1804 |
| | CUCACAAGAAUCCUGAAAAAGGAUGCCAAACGAAGGUG | |
| | GAGUAUAGAAAUA | |
| n/a | ACAGCUUAUUUGGAAGCUGAAAUGUGAGGUUUAUAACA | 1805 |
| | CUCACAAGAAUCCUGAAAAAGGAUGCCAAACCCACUAC | |
| | CCAGUGCAAGGCU | |
| n/a | ACAGCUUAUUUGGAAGCUGAAAUGUGAGGUUUAUAACA | 1806 |
| | CUCACAAGAAUCCUGAAAAAGGAUGCCAAACGGUUUCU | |
| | UUUCCUCGCUAGG | |
| n/a | ACAGCUUAUUUGGAAGCUGAAAUGUGAGGUUUAUAACA | 1807 |
| | CUCACAAGAAUCCUGAAAAAGGAUGCCAAACAGGUCGG | |
| | UGAGACAGGAAAA | |
| n/a | ACAGCUUAUUUGGAAGCUGAAAUGUGAGGUUUAUAACA | 1808 |
| | CUCACAAGAAUCCUGAAAAAGGAUGCCAAACGAGAACU | |
| | ACAGUAGACCCAG | |
| n/a | ACAGCUUAUUUGGAAGCUGAAAUGUGAGGUUUAUAACA | 1809 |
| | CUCACAAGAAUCCUGAAAAAGGAUGCCAAACAGCUGGG | |
| | CAAAGGUCACCUG | |
| n/a | ACAGCUUAUUUGGAAGCUGAAAUGUGAGGUUUAUAACA | 1810 |
| | CUCACAAGAAUCCUGAAAAAGGAUGCCAAACUUAGAUA | |
| | GAGAACUACAGUA | |
| n/a | ACAGCUUAUUUGGAAGCUGAAAUGUGAGGUUUAUAACA | 1811 |
| | CUCACAAGAAUCCUGAAAAAGGAUGCCAAACCAGGCAG | |
| | CUCUGCCACUACC | |
| n/a | ACAGCUUAUUUGGAAGCUGAAAUGUGAGGUUUAUAACA | 1812 |
| | CUCACAAGAAUCCUGAAAAAGGAUGCCAAACCAAGGCU | |
| | UUUGGCCCAUAGC | |
| n/a | ACAGCUUAUUUGGAAGCUGAAAUGUGAGGUUUAUAACA | 1813 |
| | CUCACAAGAAUCCUGAAAAAGGAUGCCAAACAGAGCUU | |
| | CUCCCUCCAGCUC | |
| n/a | ACAGCUUAUUUGGAAGCUGAAAUGUGAGGUUUAUAACA | 1814 |
| | CUCACAAGAAUCCUGAAAAAGGAUGCCAAACGGUAGGC | |
| | AUGGAUACCAAAG | |
| n/a | ACAGCUUAUUUGGAAGCUGAAAUGUGAGGUUUAUAACA | 1815 |
| | CUCACAAGAAUCCUGAAAAAGGAUGCCAAACAGAAACA | |
| | AUAGGUUUCUUUU | |
| n/a | ACAGCUUAUUUGGAAGCUGAAAUGUGAGGUUUAUAACA | 1816 |
| | CUCACAAGAAUCCUGAAAAAGGAUGCCAAACGGGUGGG | |
| | AGGGGGCAGAGUG | |
| n/a | ACAGCUUAUUUGGAAGCUGAAAUGUGAGGUUUAUAACA | 1817 |
| | CUCACAAGAAUCCUGAAAAAGGAUGCCAAACUGCUGAG | |
| | AAACAAUAGGUUU | |
| n/a | ACAGCUUAUUUGGAAGCUGAAAUGUGAGGUUUAUAACA | 1818 |
| | CUCACAAGAAUCCUGAAAAAGGAUGCCAAACGCUGGGC | |
| | AGGGAUUAGUGAG | |
| n/a | ACAGCUUAUUUGGAAGCUGAAAUGUGAGGUUUAUAACA | 1819 |
| | CUCACAAGAAUCCUGAAAAAGGAUGCCAAACAACAAGG | |
| | GACAGCAUGACCC | |
| n/a | ACAGCUUAUUUGGAAGCUGAAAUGUGAGGUUUAUAACA | 1820 |
| | CUCACAAGAAUCCUGAAAAAGGAUGCCAAACGAGCAAC | |
| | GGAGGAAGUGGGG | |
| n/a | ACAGCUUAUUUGGAAGCUGAAAUGUGAGGUUUAUAACA | 1821 |
| | CUCACAAGAAUCCUGAAAAAGGAUGCCAAACCCGGCUC | |
| | ACCUAGAUGAGGU | |
| n/a | ACAGCUUAUUUGGAAGCUGAAAUGUGAGGUUUAUAACA | 1822 |
| | CUCACAAGAAUCCUGAAAAAGGAUGCCAAACGGACUCA | |
| | GUUUUUUCAGUCC | |
| n/a | ACAGCUUAUUUGGAAGCUGAAAUGUGAGGUUUAUAACA | 1823 |
| | CUCACAAGAAUCCUGAAAAAGGAUGCCAAACGAAAUAC | |
| | UCUGCAGAACGGG | |
| n/a | ACAGCUUAUUUGGAAGCUGAAAUGUGAGGUUUAUAACA | 1824 |
| | CUCACAAGAAUCCUGAAAAAGGAUGCCAAACGGCUAGA | |
| | UGGCUGGGUGGUG | |
| n/a | ACAGCUUAUUUGGAAGCUGAAAUGUGAGGUUUAUAACA | 1825 |
| | CUCACAAGAAUCCUGAAAAAGGAUGCCAAACGGAGGGG | |
| | GCAGAGUGAAGGU | |
| n/a | ACAGCUUAUUUGGAAGCUGAAAUGUGAGGUUUAUAACA | 1826 |
| | CUCACAAGAAUCCUGAAAAAGGAUGCCAAACGCUGCUU | |
| | CUAGGGAUAAAAC | |
| n/a | ACAGCUUAUUUGGAAGCUGAAAUGUGAGGUUUAUAACA | 1827 |
| | CUCACAAGAAUCCUGAAAAAGGAUGCCAAACCCUGGAG | |
| | UAGCUAGCUGCUU | |
| n/a | ACAGCUUAUUUGGAAGCUGAAAUGUGAGGUUUAUAACA | 1828 |
| | CUCACAAGAAUCCUGAAAAAGGAUGCCAAACCCAGAGG | |
| | AGUUGAGAAAUCC | |
| n/a | ACAGCUUAUUUGGAAGCUGAAAUGUGAGGUUUAUAACA | 1829 |
| | CUCACAAGAAUCCUGAAAAAGGAUGCCAAACCAGCCAU | |
| | CUGCCAGAGGAGU | |
| n/a | ACAGCUUAUUUGGAAGCUGAAAUGUGAGGUUUAUAACA | 1830 |
| | CUCACAAGAAUCCUGAAAAAGGAUGCCAAACCCCCCAC | |
| | ACUGACCUCCACC | |
| n/a | ACAGCUUAUUUGGAAGCUGAAAUGUGAGGUUUAUAACA | 1831 |
| | CUCACAAGAAUCCUGAAAAAGGAUGCCAAACGAUAGAG | |
| | AACUACAGUAGAC | |
| n/a | ACAGCUUAUUUGGAAGCUGAAAUGUGAGGUUUAUAACA | 1832 |
| | CUCACAAGAAUCCUGAAAAAGGAUGCCAAACCAUGCCC | |
| | CCACACUGACCUC | |
| n/a | ACAGCUUAUUUGGAAGCUGAAAUGUGAGGUUUAUAACA | 1833 |
| | CUCACAAGAAUCCUGAAAAAGGAUGCCAAACUGAUCCC | |
| | AACAGUCUCCUCU | |
| n/a | ACAGCUUAUUUGGAAGCUGAAAUGUGAGGUUUAUAACA | 1834 |
| | CUCACAAGAAUCCUGAAAAAGGAUGCCAAACAUCCCAA | |
| | CAGUCUCCUCUGC | |
| n/a | ACAGCUUAUUUGGAAGCUGAAAUGUGAGGUUUAUAACA | 1835 |
| | CUCACAAGAAUCCUGAAAAAGGAUGCCAAACGAAUGGG | |
| | GAAUCUGUGGUGC | |
| n/a | ACAGCUUAUUUGGAAGCUGAAAUGUGAGGUUUAUAACA | 1836 |
| | CUCACAAGAAUCCUGAAAAAGGAUGCCAAACGCUGGGU | |
| | GGUGAGAGCUUCU | |
| n/a | ACAGCUUAUUUGGAAGCUGAAAUGUGAGGUUUAUAACA | 1837 |
| | CUCACAAGAAUCCUGAAAAAGGAUGCCAAACACCCAAU | |
| | UGCAGGCAGCUCU | |
| n/a | ACAGCUUAUUUGGAAGCUGAAAUGUGAGGUUUAUAACA | 1838 |
| | CUCACAAGAAUCCUGAAAAAGGAUGCCAAACGGACAGC | |
| | GGGCACAGAAGGC | |
| n/a | ACAGCUUAUUUGGAAGCUGAAAUGUGAGGUUUAUAACA | 1839 |
| | CUCACAAGAAUCCUGAAAAAGGAUGCCAAACGAUGAGG | |
| | UCGGUGAGACAGG | |
| n/a | ACAGCUUAUUUGGAAGCUGAAAUGUGAGGUUUAUAACA | 1840 |
| | CUCACAAGAAUCCUGAAAAAGGAUGCCAAACUGGUGCC | |
| | ACUGUGGAGCAAC | |
| n/a | ACAGCUUAUUUGGAAGCUGAAAUGUGAGGUUUAUAACA | 1841 |
| | CUCACAAGAAUCCUGAAAAAGGAUGCCAAACGGCAUGG | |
| | AUACCAAAGGCUU | |
| n/a | ACAGCUUAUUUGGAAGCUGAAAUGUGAGGUUUAUAACA | 1842 |
| | CUCACAAGAAUCCUGAAAAAGGAUGCCAAACAGCAGGC | |
| | AAGCGGGGAGGGC | |
| n/a | ACAGCUUAUUUGGAAGCUGAAAUGUGAGGUUUAUAACA | 1843 |
| | CUCACAAGAAUCCUGAAAAAGGAUGCCAAACAGUCCCA | |
| | AGCCUUCUGUGGG | |
| n/a | ACAGCUUAUUUGGAAGCUGAAAUGUGAGGUUUAUAACA | 1844 |
| | CUCACAAGAAUCCUGAAAAAGGAUGCCAAACAUAGCUC | |
| | AGAGCAAGCUAAA | |
| n/a | ACAGCUUAUUUGGAAGCUGAAAUGUGAGGUUUAUAACA | 1845 |
| | CUCACAAGAAUCCUGAAAAAGGAUGCCAAACGGGAUAA | |
| | AACUGAGCAGGCA | |
| n/a | ACAGCUUAUUUGGAAGCUGAAAUGUGAGGUUUAUAACA | 1846 |
| | CUCACAAGAAUCCUGAAAAAGGAUGCCAAACAGCAGUC | |
| | CAGACCAGAGCCU | |
| n/a | ACAGCUUAUUUGGAAGCUGAAAUGUGAGGUUUAUAACA | 1847 |
| | CUCACAAGAAUCCUGAAAAAGGAUGCCAAACUGGGCUA | |
| | GAUGGCUGGGUGG | |
| n/a | ACAGCUUAUUUGGAAGCUGAAAUGUGAGGUUUAUAACA | 1848 |
| | CUCACAAGAAUCCUGAAAAAGGAUGCCAAACGCUCAGA | |
| | GCAAGCUAAACAA | |
| n/a | ACAGCUUAUUUGGAAGCUGAAAUGUGAGGUUUAUAACA | 1849 |
| | CUCACAAGAAUCCUGAAAAAGGAUGCCAAACCUUCUAG | |
| | GGAUAAAACUGAG | |
| n/a | ACAGCUUAUUUGGAAGCUGAAAUGUGAGGUUUAUAACA | 1850 |
| | CUCACAAGAAUCCUGAAAAAGGAUGCCAAACCCCAUGC | |
| | UUUUCACGGCCAC | |
| n/a | ACAGCUUAUUUGGAAGCUGAAAUGUGAGGUUUAUAACA | 1851 |
| | CUCACAAGAAUCCUGAAAAAGGAUGCCAAACUAUUGGC | |
| | UCCAGGAUGGGAC | |
| n/a | ACAGCUUAUUUGGAAGCUGAAAUGUGAGGUUUAUAACA | 1852 |
| | CUCACAAGAAUCCUGAAAAAGGAUGCCAAACGGCAAAG | |
| | GUCACCUGCUGAG | |
| n/a | ACAGCUUAUUUGGAAGCUGAAAUGUGAGGUUUAUAACA | 1853 |
| | CUCACAAGAAUCCUGAAAAAGGAUGCCAAACCAGCCUA | |
| | GAGCCAGUGACAG | |
| n/a | ACAGCUUAUUUGGAAGCUGAAAUGUGAGGUUUAUAACA | 1854 |
| | CUCACAAGAAUCCUGAAAAAGGAUGCCAAACAAAAGCA | |
| | UGGGCAAUCUCAG | |
| n/a | ACAGCUUAUUUGGAAGCUGAAAUGUGAGGUUUAUAACA | 1855 |
| | CUCACAAGAAUCCUGAAAAAGGAUGCCAAACCUCCAGG | |
| | UAAUGCCCCUGGG | |
| n/a | ACAGCUUAUUUGGAAGCUGAAAUGUGAGGUUUAUAACA | 1856 |
| | CUCACAAGAAUCCUGAAAAAGGAUGCCAAACGCUACUC | |
| | CAGGUAAUGCCCC | |
| n/a | ACAGCUUAUUUGGAAGCUGAAAUGUGAGGUUUAUAACA | 1857 |
| | CUCACAAGAAUCCUGAAAAAGGAUGCCAAACUCCCCUG | |
| | UCUUUUCCUGUCU | |
| n/a | ACAGCUUAUUUGGAAGCUGAAAUGUGAGGUUUAUAACA | 1858 |
| | CUCACAAGAAUCCUGAAAAAGGAUGCCAAACUGCCCGC | |
| | UGUCCCAUCCUGG | |
| n/a | ACAGCUUAUUUGGAAGCUGAAAUGUGAGGUUUAUAACA | 1859 |
| | CUCACAAGAAUCCUGAAAAAGGAUGCCAAACCUCAGUU | |
| | UUAUCCCUAGAAG | |
| n/a | ACAGCUUAUUUGGAAGCUGAAAUGUGAGGUUUAUAACA | 1860 |
| | CUCACAAGAAUCCUGAAAAAGGAUGCCAAACCAGAGUA | |
| | UUUCUAUACUCCA | |
| n/a | ACAGCUUAUUUGGAAGCUGAAAUGUGAGGUUUAUAACA | 1861 |
| | CUCACAAGAAUCCUGAAAAAGGAUGCCAAACCUGUCCC | |
| | UUGUUUAGCUUGC | |
| n/a | ACAGCUUAUUUGGAAGCUGAAAUGUGAGGUUUAUAACA | 1862 |
| | CUCACAAGAAUCCUGAAAAAGGAUGCCAAACGGUGAGC | |
| | CGGUAGCUGAUCC | |
| n/a | ACAGCUUAUUUGGAAGCUGAAAUGUGAGGUUUAUAACA | 1863 |
| | CUCACAAGAAUCCUGAAAAAGGAUGCCAAACCUGGAAG | |
| | GCUUUCAGGUGGC | |
| n/a | ACAGCUUAUUUGGAAGCUGAAAUGUGAGGUUUAUAACA | 1864 |
| | CUCACAAGAAUCCUGAAAAAGGAUGCCAAACGGCCAAA | |
| | AGCCUUGCACUGG | |
| n/a | ACAGCUUAUUUGGAAGCUGAAAUGUGAGGUUUAUAACA | 1865 |
| | CUCACAAGAAUCCUGAAAAAGGAUGCCAAACCCCCUGG | |
| | GGAGGAGAGGAAG | |
| n/a | ACAGCUUAUUUGGAAGCUGAAAUGUGAGGUUUAUAACA | 1866 |
| | CUCACAAGAAUCCUGAAAAAGGAUGCCAAACCUGUAGU | |
| | UCUCUAUCUAAUA | |
| n/a | ACAGCUUAUUUGGAAGCUGAAAUGUGAGGUUUAUAACA | 1867 |
| | CUCACAAGAAUCCUGAAAAAGGAUGCCAAACGGUCUAC | |
| | UGUAGUUCUCUAU | |
| n/a | ACAGCUUAUUUGGAAGCUGAAAUGUGAGGUUUAUAACA | 1868 |
| | CUCACAAGAAUCCUGAAAAAGGAUGCCAAACUCUAAUA | |
| | UCAGUGGGAGAAA | |
| n/a | ACAGCUUAUUUGGAAGCUGAAAUGUGAGGUUUAUAACA | 1869 |
| | CUCACAAGAAUCCUGAAAAAGGAUGCCAAACAUCCCUU | |
| | GGUGGCGGAGGUG | |
| n/a | ACAGCUUAUUUGGAAGCUGAAAUGUGAGGUUUAUAACA | 1870 |
| | CUCACAAGAAUCCUGAAAAAGGAUGCCAAACGAUGUCC | |
| | CGUUCUGCAGAGU | |
| n/a | ACAGCUUAUUUGGAAGCUGAAAUGUGAGGUUUAUAACA | 1871 |
| | CUCACAAGAAUCCUGAAAAAGGAUGCCAAACCCUGCUC | |
| | AGUUUUAUCCCUA | |
| n/a | ACAGCUUAUUUGGAAGCUGAAAUGUGAGGUUUAUAACA | 1872 |
| | CUCACAAGAAUCCUGAAAAAGGAUGCCAAACAAACAGG | |
| | UCACAGCCCUCCC | |
| n/a | ACAGCUUAUUUGGAAGCUGAAAUGUGAGGUUUAUAACA | 1873 |
| | CUCACAAGAAUCCUGAAAAAGGAUGCCAAACUCACUGG | |
| | CUCUAGGCUGUAA | |
| n/a | ACAGCUUAUUUGGAAGCUGAAAUGUGAGGUUUAUAACA | 1874 |
| | CUCACAAGAAUCCUGAAAAAGGAUGCCAAACCUCUGAG | |
| | CUAUUAGAAGCCU | |
| n/a | ACAGCUUAUUUGGAAGCUGAAAUGUGAGGUUUAUAACA | 1875 |
| | CUCACAAGAAUCCUGAAAAAGGAUGCCAAACAUCCCUG | |
| | CCCAGCUACGGCA | |
| n/a | ACAGCUUAUUUGGAAGCUGAAAUGUGAGGUUUAUAACA | 1876 |
| | CUCACAAGAAUCCUGAAAAAGGAUGCCAAACGCUUCUG | |
| | UGAAGGAGCCUGU | |
| n/a | ACAGCUUAUUUGGAAGCUGAAAUGUGAGGUUUAUAACA | 1877 |
| | CUCACAAGAAUCCUGAAAAAGGAUGCCAAACUAGUUCU | |
| | CUAUCUAAUAUCA | |
| n/a | ACAGCUUAUUUGGAAGCUGAAAUGUGAGGUUUAUAACA | 1878 |
| | CUCACAAGAAUCCUGAAAAAGGAUGCCAAACCGGCAGA | |
| | GGAGACUGUUGGG | |
| n/a | ACAGCUUAUUUGGAAGCUGAAAUGUGAGGUUUAUAACA | 1879 |
| | CUCACAAGAAUCCUGAAAAAGGAUGCCAAACAAGGAGC | |
| | CUGUCACUGGCUC | |
| n/a | ACAGCUUAUUUGGAAGCUGAAAUGUGAGGUUUAUAACA | 1880 |
| | CUCACAAGAAUCCUGAAAAAGGAUGCCAAACGCCCACA | |
| | GAAGGCUUGGGAC | |
| n/a | ACAGCUUAUUUGGAAGCUGAAAUGUGAGGUUUAUAACA | 1881 |
| | CUCACAAGAAUCCUGAAAAAGGAUGCCAAACUCCCUAG | |
| | AAGCAGCUAGCUA | |
| n/a | ACAGCUUAUUUGGAAGCUGAAAUGUGAGGUUUAUAACA | 1882 |
| | CUCACAAGAAUCCUGAAAAAGGAUGCCAAACCCUACCC | |
| | AGGACUGAAAAAA | |
| n/a | ACAGCUUAUUUGGAAGCUGAAAUGUGAGGUUUAUAACA | 1883 |
| | CUCACAAGAAUCCUGAAAAAGGAUGCCAAACUUGUUUC | |
| | UCAGCAUAUUAGA | |
| n/a | ACAGCUUAUUUGGAAGCUGAAAUGUGAGGUUUAUAACA | 1884 |
| | CUCACAAGAAUCCUGAAAAAGGAUGCCAAACUUGGGAU | |
| | CACAGGUGGAGGU | |
| n/a | ACAGCUUAUUUGGAAGCUGAAAUGUGAGGUUUAUAACA | 1885 |
| | CUCACAAGAAUCCUGAAAAAGGAUGCCAAACGCAGAUG | |
| | GCUGCAUGGCCUU | |
| n/a | ACAGCUUAUUUGGAAGCUGAAAUGUGAGGUUUAUAACA | 1886 |
| | CUCACAAGAAUCCUGAAAAAGGAUGCCAAACCCUCAGG | |
| | CUCUGGUCUGGAC | |
| n/a | ACAGCUUAUUUGGAAGCUGAAAUGUGAGGUUUAUAACA | 1887 |
| | CUCACAAGAAUCCUGAAAAAGGAUGCCAAACACCUUUG | |
| | CCCAGCUCACUGG | |
| n/a | ACAGCUUAUUUGGAAGCUGAAAUGUGAGGUUUAUAACA | 1888 |
| | CUCACAAGAAUCCUGAAAAAGGAUGCCAAACGUAUCCA | |
| | UGCCUACCCAGGA | |
| n/a | ACAGCUUAUUUGGAAGCUGAAAUGUGAGGUUUAUAACA | 1889 |
| | CUCACAAGAAUCCUGAAAAAGGAUGCCAAACGAAGCCU | |
| | UUGGUAUCCAUGC | |
| n/a | ACAGCUUAUUUGGAAGCUGAAAUGUGAGGUUUAUAACA | 1890 |
| | CUCACAAGAAUCCUGAAAAAGGAUGCCAAACGGGGCAU | |
| | GUAACCUUCACUC | |
| n/a | ACAGCUUAUUUGGAAGCUGAAAUGUGAGGUUUAUAACA | 1891 |
| | CUCACAAGAAUCCUGAAAAAGGAUGCCAAACGGGAAAG | |
| | GGAGGCUAUGGGC | |
| n/a | ACAGCUUAUUUGGAAGCUGAAAUGUGAGGUUUAUAACA | 1892 |
| | CUCACAAGAAUCCUGAAAAAGGAUGCCAAACUGAAGGA | |
| | GCCUGUCACUGGC | |
| n/a | ACAGCUUAUUUGGAAGCUGAAAUGUGAGGUUUAUAACA | 1893 |
| | CUCACAAGAAUCCUGAAAAAGGAUGCCAAACGGGGGGG | |
| | CUAGAAGCUGGGG | |
| n/a | ACAGCUUAUUUGGAAGCUGAAAUGUGAGGUUUAUAACA | 1894 |
| | CUCACAAGAAUCCUGAAAAAGGAUGCCAAACGGUAGUG | |
| | GCAGAGCUGCCUG | |
| n/a | ACAGCUUAUUUGGAAGCUGAAAUGUGAGGUUUAUAACA | 1895 |
| | CUCACAAGAAUCCUGAAAAAGGAUGCCAAACUCCCAUC | |
| | CUGGAGCCAAUAU | |
| n/a | ACAGCUUAUUUGGAAGCUGAAAUGUGAGGUUUAUAACA | 1896 |
| | CUCACAAGAAUCCUGAAAAAGGAUGCCAAACGAAGCUG | |
| | GGGAAAGGGAGGC | |
| n/a | ACAGCUUAUUUGGAAGCUGAAAUGUGAGGUUUAUAACA | 1897 |
| | CUCACAAGAAUCCUGAAAAAGGAUGCCAAACGCUGAUC | |
| | CCUUGGUGGCGGA | |
| n/a | ACAGCUUAUUUGGAAGCUGAAAUGUGAGGUUUAUAACA | 1898 |
| | CUCACAAGAAUCCUGAAAAAGGAUGCCAAACGCGAGGA | |
| | AAAGAAACCUAUU | |
| n/a | ACAGCUUAUUUGGAAGCUGAAAUGUGAGGUUUAUAACA | 1899 |
| | CUCACAAGAAUCCUGAAAAAGGAUGCCAAACGACUGCU | |
| | CAGCAGGUGACCU | |
| n/a | ACAGCUUAUUUGGAAGCUGAAAUGUGAGGUUUAUAACA | 1900 |
| | CUCACAAGAAUCCUGAAAAAGGAUGCCAAACGAAGGCU | |
| | UUCAGGUGGCUUC | |
| n/a | ACAGCUUAUUUGGAAGCUGAAAUGUGAGGUUUAUAACA | 1901 |
| | CUCACAAGAAUCCUGAAAAAGGAUGCCAAACAGGUCCA | |
| | AGGCUUGUCCCCU | |
| n/a | ACAGCUUAUUUGGAAGCUGAAAUGUGAGGUUUAUAACA | 1902 |
| | CUCACAAGAAUCCUGAAAAAGGAUGCCAAACUGGGGGG | |
| | GGCUAGAAGCUGG | |
| n/a | ACAGCUUAUUUGGAAGCUGAAAUGUGAGGUUUAUAACA | 1903 |
| | CUCACAAGAAUCCUGAAAAAGGAUGCCAAACUUUCUCA | |
| | GCAUAUUAGAGUA | |
| n/a | ACAGCUUAUUUGGAAGCUGAAAUGUGAGGUUUAUAACA | 1904 |
| | CUCACAAGAAUCCUGAAAAAGGAUGCCAAACGGCAAUC | |
| | UCAGAGGGAUUUC | |
| n/a | ACAGCUUAUUUGGAAGCUGAAAUGUGAGGUUUAUAACA | 1905 |
| | CUCACAAGAAUCCUGAAAAAGGAUGCCAAACAAGCAGG | |
| | CCUGAGGUCCAAG | |
| n/a | ACAGCUUAUUUGGAAGCUGAAAUGUGAGGUUUAUAACA | 1906 |
| | CUCACAAGAAUCCUGAAAAAGGAUGCCAAACUCUCACC | |
| | GACCUCAUCUAGG | |
| n/a | ACAGCUUAUUUGGAAGCUGAAAUGUGAGGUUUAUAACA | 1907 |
| | CUCACAAGAAUCCUGAAAAAGGAUGCCAAACUCCCGUU | |
| | CUGCAGAGUAUUU | |
| n/a | ACAGCUUAUUUGGAAGCUGAAAUGUGAGGUUUAUAACA | 1908 |
| | CUCACAAGAAUCCUGAAAAAGGAUGCCAAACUCAGUGG | |
| | GAGAAAGGCUUGG | |
| n/a | ACAGCUUAUUUGGAAGCUGAAAUGUGAGGUUUAUAACA | 1909 |
| | CUCACAAGAAUCCUGAAAAAGGAUGCCAAACGAAGCAG | |
| | CUAGCUACUCCAG | |
| n/a | ACAGCUUAUUUGGAAGCUGAAAUGUGAGGUUUAUAACA | 1910 |
| | CUCACAAGAAUCCUGAAAAAGGAUGCCAAACAUAUCAG | |
| | UGGGAGAAAGGCU | |
| n/a | ACAGCUUAUUUGGAAGCUGAAAUGUGAGGUUUAUAACA | 1911 |
| | CUCACAAGAAUCCUGAAAAAGGAUGCCAAACAUGCCCC | |
| | UGGGGAGGAGAGG | |
| n/a | ACAGCUUAUUUGGAAGCUGAAAUGUGAGGUUUAUAACA | 1912 |
| | CUCACAAGAAUCCUGAAAAAGGAUGCCAAACUCCCUUG | |
| | UUUAGCUUGCUCU | |
| n/a | ACAGCUUAUUUGGAAGCUGAAAUGUGAGGUUUAUAACA | 1913 |
| | CUCACAAGAAUCCUGAAAAAGGAUGCCAAACCCCGCUG | |
| | UCCCAUCCUGGAG | |
| n/a | ACAGCUUAUUUGGAAGCUGAAAUGUGAGGUUUAUAACA | 1914 |
| | CUCACAAGAAUCCUGAAAAAGGAUGCCAAACGAGCCAA | |
| | UAUAAAACAGGUC | |
| n/a | ACAGCUUAUUUGGAAGCUGAAAUGUGAGGUUUAUAACA | 1915 |
| | CUCACAAGAAUCCUGAAAAAGGAUGCCAAACGCCUUCC | |
| | UGCCUCAGGCUCU | |
| n/a | ACAGCUUAUUUGGAAGCUGAAAUGUGAGGUUUAUAACA | 1916 |
| | CUCACAAGAAUCCUGAAAAAGGAUGCCAAACGGCUGUA | |
| | AAGCAGGCCUGAG | |
| n/a | ACAGCUUAUUUGGAAGCUGAAAUGUGAGGUUUAUAACA | 1917 |
| | CUCACAAGAAUCCUGAAAAAGGAUGCCAAACUAAAGCA | |
| | GGCCUGAGGUCCA | |
| n/a | ACAGCUUAUUUGGAAGCUGAAAUGUGAGGUUUAUAACA | 1918 |
| | CUCACAAGAAUCCUGAAAAAGGAUGCCAAACGCGGAGG | |
| | UGGCCGUGAAAAG | |
| n/a | ACAGCUUAUUUGGAAGCUGAAAUGUGAGGUUUAUAACA | 1919 |
| | CUCACAAGAAUCCUGAAAAAGGAUGCCAAACAGCCGGU | |
| | AGCUGAUCCCUUG | |
| n/a | ACAGCUUAUUUGGAAGCUGAAAUGUGAGGUUUAUAACA | 1920 |
| | CUCACAAGAAUCCUGAAAAAGGAUGCCAAACGUGGCGG | |
| | AGGUGGCCGUGAA | |
| n/a | ACAGCUUAUUUGGAAGCUGAAAUGUGAGGUUUAUAACA | 1921 |
| | CUCACAAGAAUCCUGAAAAAGGAUGCCAAACUACUCCA | |
| | CCUUCCACCCCAC | |
| n/a | ACAGCUUAUUUGGAAGCUGAAAUGUGAGGUUUAUAACA | 1922 |
| | CUCACAAGAAUCCUGAAAAAGGAUGCCAAACGUUCUCU | |
| | AUCUAAUAUCAGU | |
| n/a | ACAGCUUAUUUGGAAGCUGAAAUGUGAGGUUUAUAACA | 1923 |
| | CUCACAAGAAUCCUGAAAAAGGAUGCCAAACCCCAGCU | |
| | CACUGGGCCUUCU | |
| n/a | ACAGCUUAUUUGGAAGCUGAAAUGUGAGGUUUAUAACA | 1924 |
| | CUCACAAGAAUCCUGAAAAAGGAUGCCAAACUGGGCCA | |
| | AAAGCCUUGCACU | |
| n/a | ACAGCUUAUUUGGAAGCUGAAAUGUGAGGUUUAUAACA | 1925 |
| | CUCACAAGAAUCCUGAAAAAGGAUGCCAAACCUCCACC | |
| | UUCCACCCCACUU | |
| n/a | ACAGCUUAUUUGGAAGCUGAAAUGUGAGGUUUAUAACA | 1926 |
| | CUCACAAGAAUCCUGAAAAAGGAUGCCAAACGAGGUCA | |
| | GUGUGGGGGCAUG | |
| n/a | ACAGCUUAUUUGGAAGCUGAAAUGUGAGGUUUAUAACA | 1927 |
| | CUCACAAGAAUCCUGAAAAAGGAUGCCAAACCACUGGG | |
| | UAGUGGCAGAGCU | |
| n/a | ACAGCUUAUUUGGAAGCUGAAAUGUGAGGUUUAUAACA | 1928 |
| | CUCACAAGAAUCCUGAAAAAGGAUGCCAAACCCCAGGA | |
| | CUGAAAAAACUGA | |
| n/a | ACAGCUUAUUUGGAAGCUGAAAUGUGAGGUUUAUAACA | 1929 |
| | CUCACAAGAAUCCUGAAAAAGGAUGCCAAACCCUGCAA | |
| | UUGGGUCAUGCUG | |
| n/a | ACAGCUUAUUUGGAAGCUGAAAUGUGAGGUUUAUAACA | 1930 |
| | CUCACAAGAAUCCUGAAAAAGGAUGCCAAACCCCCCUC | |
| | CCACCCCACUUCC | |
| n/a | ACAGCUUAUUUGGAAGCUGAAAUGUGAGGUUUAUAACA | 1931 |
| | CUCACAAGAAUCCUGAAAAAGGAUGCCAAACGGAGAAA | |
| | GGCUUGGGAUUCA | |
| n/a | ACAGCUUAUUUGGAAGCUGAAAUGUGAGGUUUAUAACA | 1932 |
| | CUCACAAGAAUCCUGAAAAAGGAUGCCAAACUAACCUU | |
| | CACUCUGCCCCCU | |
| n/a | ACAGCUUAUUUGGAAGCUGAAAUGUGAGGUUUAUAACA | 1933 |
| | CUCACAAGAAUCCUGAAAAAGGAUGCCAAACCAUGGCC | |
| | UUCCUGCCUCAGG | |
| n/a | ACAGCUUAUUUGGAAGCUGAAAUGUGAGGUUUAUAACA | 1934 |
| | CUCACAAGAAUCCUGAAAAAGGAUGCCAAACUCCAUGC | |
| | CUACCCAGGACUG | |
| n/a | ACAGCUUAUUUGGAAGCUGAAAUGUGAGGUUUAUAACA | 1935 |
| | CUCACAAGAAUCCUGAAAAAGGAUGCCAAACCUCCACA | |
| | GUGGCACCACAGA | |
| n/a | ACAGCUUAUUUGGAAGCUGAAAUGUGAGGUUUAUAACA | 1936 |
| | CUCACAAGAAUCCUGAAAAAGGAUGCCAAACGGCCUUC | |
| | UGUGCCCGCUGUC | |
| n/a | ACAGCUUAUUUGGAAGCUGAAAUGUGAGGUUUAUAACA | 1937 |
| | CUCACAAGAAUCCUGAAAAAGGAUGCCAAACGUCUGGA | |
| | CUGCUCAGCAGGU | |
| n/a | ACAGCUUAUUUGGAAGCUGAAAUGUGAGGUUUAUAACA | 1938 |
| | CUCACAAGAAUCCUGAAAAAGGAUGCCAAACCUCAGCA | |
| | GGUGACCUUUGCC | |
| n/a | ACAGCUUAUUUGGAAGCUGAAAUGUGAGGUUUAUAACA | 1939 |
| | CUCACAAGAAUCCUGAAAAAGGAUGCCAAACGCUUGCU | |
| | CUGAGCUAUUAGA | |
| n/a | ACAGCUUAUUUGGAAGCUGAAAUGUGAGGUUUAUAACA | 1940 |
| | CUCACAAGAAUCCUGAAAAAGGAUGCCAAACUCCUUCU | |
| | CUCACUAAUCCCU | |
| n/a | ACAGCUUAUUUGGAAGCUGAAAUGUGAGGUUUAUAACA | 1941 |
| | CUCACAAGAAUCCUGAAAAAGGAUGCCAAACUUAGAGU | |
| | AGAUGUCCCGUUC | |
| n/a | ACAGCUUAUUUGGAAGCUGAAAUGUGAGGUUUAUAACA | 1942 |
| | CUCACAAGAAUCCUGAAAAAGGAUGCCAAACUAAAACA | |
| | GGUCACAGCCCUC | |
| n/a | ACAGCUUAUUUGGAAGCUGAAAUGUGAGGUUUAUAACA | 1943 |
| | CUCACAAGAAUCCUGAAAAAGGAUGCCAAACAGUCCUA | |
| | GCGAGGAAAAGAA | |
| n/a | ACAGCUUAUUUGGAAGCUGAAAUGUGAGGUUUAUAACA | 1944 |
| | CUCACAAGAAUCCUGAAAAAGGAUGCCAAACGAGGGAG | |
| | AAGCUCUCACCAC | |
| n/a | ACAGCUUAUUUGGAAGCUGAAAUGUGAGGUUUAUAACA | 1945 |
| | CUCACAAGAAUCCUGAAAAAGGAUGCCAAACGUCUCCA | |
| | CCCUUGGGUUCCU | |
| n/a | ACAGCUUAUUUGGAAGCUGAAAUGUGAGGUUUAUAACA | 1946 |
| | CUCACAAGAAUCCUGAAAAAGGAUGCCAAACCCCAGCU | |
| | ACGGCAGAGGAGA | |
| n/a | ACAGCUUAUUUGGAAGCUGAAAUGUGAGGUUUAUAACA | 1947 |
| | CUCACAAGAAUCCUGAAAAAGGAUGCCAAACUUUAGCU | |
| | UGCUCUGAGCUAU | |
| n/a | ACAGCUUAUUUGGAAGCUGAAAUGUGAGGUUUAUAACA | 1948 |
| | CUCACAAGAAUCCUGAAAAAGGAUGCCAAACGGACUCA | |
| | UGGUCUCCACCCU | |
| n/a | ACAGCUUAUUUGGAAGCUGAAAUGUGAGGUUUAUAACA | 1949 |
| | CUCACAAGAAUCCUGAAAAAGGAUGCCAAACGGUCAUG | |
| | CUGUCCCUUGUUU | |
| n/a | ACAGCUUAUUUGGAAGCUGAAAUGUGAGGUUUAUAACA | 1950 |
| | CUCACAAGAAUCCUGAAAAAGGAUGCCAAACGCCGUGA | |
| | AAAGCAUGGGCAA | |
| n/a | ACAGCUUAUUUGGAAGCUGAAAUGUGAGGUUUAUAACA | 1951 |
| | CUCACAAGAAUCCUGAAAAAGGAUGCCAAACUUAGAAG | |
| | CCUUUGGUAUCCA | |
| n/a | ACAGCUUAUUUGGAAGCUGAAAUGUGAGGUUUAUAACA | 1952 |
| | CUCACAAGAAUCCUGAAAAAGGAUGCCAAACGGAUCAC | |
| | AGGUGGAGGUCAG | |
| n/a | ACAGCUUAUUUGGAAGCUGAAAUGUGAGGUUUAUAACA | 1953 |
| | CUCACAAGAAUCCUGAAAAAGGAUGCCAAACCAAUUGG | |
| | GUCAUGCUGUCCC | |
| n/a | ACAGCUUAUUUGGAAGCUGAAAUGUGAGGUUUAUAACA | 1954 |
| | CUCACAAGAAUCCUGAAAAAGGAUGCCAAACGCUCUAG | |
| | GCUGUAAAGCAGG | |
| n/a | ACAGCUUAUUUGGAAGCUGAAAUGUGAGGUUUAUAACA | 1955 |
| | CUCACAAGAAUCCUGAAAAAGGAUGCCAAACGGUUCCU | |
| | GGUGUGGGGGGGG | |
| n/a | ACAGCUUAUUUGGAAGCUGAAAUGUGAGGUUUAUAACA | 1956 |
| | CUCACAAGAAUCCUGAAAAAGGAUGCCAAACGUGGCAG | |
| | AGCUGCCUGCAAU | |
| n/a | ACAGCUUAUUUGGAAGCUGAAAUGUGAGGUUUAUAACA | 1957 |
| | CUCACAAGAAUCCUGAAAAAGGAUGCCAAACGCACCAC | |
| | AGAUUCCCCAUUC | |
| n/a | ACAGCUUAUUUGGAAGCUGAAAUGUGAGGUUUAUAACA | 1958 |
| | CUCACAAGAAUCCUGAAAAAGGAUGCCAAACUUUCUAU | |
| | ACUCCACCUUCCA | |
| n/a | ACAGCUUAUUUGGAAGCUGAAAUGUGAGGUUUAUAACA | 1959 |
| | CUCACAAGAAUCCUGAAAAAGGAUGCCAAACGUGUGGG | |
| | GGGGGCUAGAAGC | |
| n/a | ACAGCUUAUUUGGAAGCUGAAAUGUGAGGUUUAUAACA | 1960 |
| | CUCACAAGAAUCCUGAAAAAGGAUGCCAAACACCUUCA | |
| | CUCUGCCCCCUCC | |
| n/a | ACAGCUUAUUUGGAAGCUGAAAUGUGAGGUUUAUAACA | 1961 |
| | CUCACAAGAAUCCUGAAAAAGGAUGCCAAACGCUGCAU | |
| | GGCCUUCCUGCCU | |
| n/a | ACAGCUUAUUUGGAAGCUGAAAUGUGAGGUUUAUAACA | 1962 |
| | CUCACAAGAAUCCUGAAAAAGGAUGCCAAACUGGGGGC | |
| | AUGUAACCUUCAC | |
| n/a | ACAGCUUAUUUGGAAGCUGAAAUGUGAGGUUUAUAACA | 1963 |
| | CUCACAAGAAUCCUGAAAAAGGAUGCCAAACGGGGGCU | |
| | GGGUCUACUGUAG | |
| n/a | ACAGCUUAUUUGGAAGCUGAAAUGUGAGGUUUAUAACA | 1964 |
| | CUCACAAGAAUCCUGAAAAAGGAUGCCAAACGCAGAGC | |
| | UGCCUGCAAUUGG | |
| n/a | ACAGCUUAUUUGGAAGCUGAAAUGUGAGGUUUAUAACA | 1965 |
| | CUCACAAGAAUCCUGAAAAAGGAUGCCAAACAAAAAAC | |
| | UGAGUCCUAGCGA | |
| n/a | ACAGCUUAUUUGGAAGCUGAAAUGUGAGGUUUAUAACA | 1966 |
| | CUCACAAGAAUCCUGAAAAAGGAUGCCAAACAGCUAUU | |
| | AGAAGCCUUUGGU | |
| n/a | ACAGCUUAUUUGGAAGCUGAAAUGUGAGGUUUAUAACA | 1967 |
| | CUCACAAGAAUCCUGAAAAAGGAUGCCAAACGGGAGGA | |
| | GAGGAAGGAAGGG | |
| n/a | ACAGCUUAUUUGGAAGCUGAAAUGUGAGGUUUAUAACA | 1968 |
| | CUCACAAGAAUCCUGAAAAAGGAUGCCAAACUCUUUUC | |
| | CUGUCUCACCGAC | |
| n/a | ACAGCUUAUUUGGAAGCUGAAAUGUGAGGUUUAUAACA | 1969 |
| | CUCACAAGAAUCCUGAAAAAGGAUGCCAAACGAGUAGA | |
| | UGUCCCGUUCUGC | |
| PL34554 | ACAGCUUAUUUGGAAGCUGAAAUGUGAGGUUUAUAACA | 2075 |
| | CUCACAAGAAUCCUGAAAAAGGAUGCCAAACCCUCGCC | |
| | GCGGCACAGGUGG | |
| PL34555 | ACAGCUUAUUUGGAAGCUGAAAUGUGAGGUUUAUAACA | 2076 |
| | CUCACAAGAAUCCUGAAAAAGGAUGCCAAACCCAGGCA | |
| | ACCUCCACGGAUC | |
| PL34556 | ACAGCUUAUUUGGAAGCUGAAAUGUGAGGUUUAUAACA | 2077 |
| | CUCACAAGAAUCCUGAAAAAGGAUGCCAAACGCGACCU | |
| | GCUGGAGCUGGUG | |
| PL34557 | ACAGCUUAUUUGGAAGCUGAAAUGUGAGGUUUAUAACA | 2078 |
| | CUCACAAGAAUCCUGAAAAAGGAUGCCAAACAGUGGCG | |
| | ACCUGCUGGAGCU | |
| PL34558 | ACAGCUUAUUUGGAAGCUGAAAUGUGAGGUUUAUAACA | 2079 |
| | CUCACAAGAAUCCUGAAAAAGGAUGCCAAACACUGUCA | |
| | CACUUGCUGGCCU | |
| PL34559 | ACAGCUUAUUUGGAAGCUGAAAUGUGAGGUUUAUAACA | 2080 |
| | CUCACAAGAAUCCUGAAAAAGGAUGCCAAACCUCCCCA | |
| | GCCUCAGCUCCCG | |
| PL34560 | ACAGCUUAUUUGGAAGCUGAAAUGUGAGGUUUAUAACA | 2081 |
| | CUCACAAGAAUCCUGAAAAAGGAUGCCAAACGCCCCAA | |
| | CUGUGAUGACCUG | |
| PL34561 | ACAGCUUAUUUGGAAGCUGAAAUGUGAGGUUUAUAACA | 2082 |
| | CUCACAAGAAUCCUGAAAAAGGAUGCCAAACCCCCCCA | |
| | GCACCCAUGGGGC | |
| PL34562 | ACAGCUUAUUUGGAAGCUGAAAUGUGAGGUUUAUAACA | 2083 |
| | CUCACAAGAAUCCUGAAAAAGGAUGCCAAACCAAAACA | |
| | GCUGCCAACCUGC | |
| R16925 | ACAGCUUAUUUGGAAGCUGAAAUGUGAGGUUUAUAACA | 2087 |
| | CUCACAAGAAUCCUGAAAAAGGAUGCCAAACACCCUGC | |
| | AUGAAGCUGAGAA | |
| R16926 | ACAGCUUAUUUGGAAGCUGAAAUGUGAGGUUUAUAACA | 2088 |
| | CUCACAAGAAUCCUGAAAAAGGAUGCCAAACGGAUUUG | |
| | GACCCUGAGGUCA | |
| R11498 | ACAGCUUAUUUGGAAGCUGAAAUGUGAGGUUUAUAACA | 2089 |
| | CUCACAAGAAUCCUGAAAAAGGAUGCCAAACGUACAAG | |
| | AGAUAGAAAGACC | |

TABLE 12
Exemplary Guide Nucleic Acids Targeting PCSK9 for CasM.265466
Effector Proteins

| | | SEQ ID |
|---|---|---|
| Guide ID | Guide sequence (shown as RNA), 5′- 3′ | NO: |
| R18133 | ACAGCUUAUUUGGAAGCUGAAAUGUGAGGUUUAUAACA | 585 |
| | CUCACAAGAAUCCUGAAAAAGGAUGCCAAACGAAGCGG | |
| | GUCCCGUCCUCCU | |
| R18134 | ACAGCUUAUUUGGAAGCUGAAAUGUGAGGUUUAUAACA | 586 |
| | CUCACAAGAAUCCUGAAAAAGGAUGCCAAACUCUAGGA | |
| | GAUACACCUCCAC | |
| R18135 | ACAGCUUAUUUGGAAGCUGAAAUGUGAGGUUUAUAACA | 587 |
| | CUCACAAGAAUCCUGAAAAAGGAUGCCAAACACCAUGA | |
| | CCCUGCCCUCGAU | |
| R18136 | ACAGCUUAUUUGGAAGCUGAAAUGUGAGGUUUAUAACA | 588 |
| | CUCACAAGAAUCCUGAAAAAGGAUGCCAAACACCCUGC | |
| | CCUCGAUUUCCCG | |
| R18137 | ACAGCUUAUUUGGAAGCUGAAAUGUGAGGUUUAUAACA | 589 |
| | CUCACAAGAAUCCUGAAAAAGGAUGCCAAACCCCUCGA | |
| | UUUCCCGGUGGUC | |
| R18138 | ACAGCUUAUUUGGAAGCUGAAAUGUGAGGUUUAUAACA | 590 |
| | CUCACAAGAAUCCUGAAAAAGGAUGCCAAACUAUGCUG | |
| | GUGUCUAGGAGAU | |
| R18139 | ACAGCUUAUUUGGAAGCUGAAAUGUGAGGUUUAUAACA | 591 |
| | CUCACAAGAAUCCUGAAAAAGGAUGCCAAACUGCUGGU | |
| | GUCUAGGAGAUAC | |
| R18140 | ACAGCUUAUUUGGAAGCUGAAAUGUGAGGUUUAUAACA | 592 |
| | CUCACAAGAAUCCUGAAAAAGGAUGCCAAACGUCACUC | |
| | UGUAUGCUGGUGU | |
| R18141 | ACAGCUUAUUUGGAAGCUGAAAUGUGAGGUUUAUAACA | 593 |
| | CUCACAAGAAUCCUGAAAAAGGAUGCCAAACUGGAAGC | |
| | GGGUCCCGUCCUC | |
| R18142 | ACAGCUUAUUUGGAAGCUGAAAUGUGAGGUUUAUAACA | 594 |
| | CUCACAAGAAUCCUGAAAAAGGAUGCCAAACCUGGUGU | |
| | CUAGGAGAUACAC | |
| R18143 | ACAGCUUAUUUGGAAGCUGAAAUGUGAGGUUUAUAACA | 595 |
| | CUCACAAGAAUCCUGAAAAAGGAUGCCAAACGGAGAUA | |
| | CACCUCCACCAGG | |
| R18144 | ACAGCUUAUUUGGAAGCUGAAAUGUGAGGUUUAUAACA | 596 |
| | CUCACAAGAAUCCUGAAAAAGGAUGCCAAACGUGUCUA | |
| | GGAGAUACACCUC | |
| R18145 | ACAGCUUAUUUGGAAGCUGAAAUGUGAGGUUUAUAACA | 597 |
| | CUCACAAGAAUCCUGAAAAAGGAUGCCAAACCACCUCC | |
| | ACCAGGCUGCCUC | |
| R18146 | ACAGCUUAUUUGGAAGCUGAAAUGUGAGGUUUAUAACA | 598 |
| | CUCACAAGAAUCCUGAAAAAGGAUGCCAAACGACACCA | |
| | GCAUACAGAGUGA | |
| R18147 | ACAGCUUAUUUGGAAGCUGAAAUGUGAGGUUUAUAACA | 599 |
| | CUCACAAGAAUCCUGAAAAAGGAUGCCAAACUGCCCGA | |
| | GGAGGACGGGACC | |
| R18148 | ACAGCUUAUUUGGAAGCUGAAAUGUGAGGUUUAUAACA | 600 |
| | CUCACAAGAAUCCUGAAAAAGGAUGCCAAACGUGGAGG | |
| | UGUAUCUCCUAGA | |
| R18149 | ACAGCUUAUUUGGAAGCUGAAAUGUGAGGUUUAUAACA | 601 |
| | CUCACAAGAAUCCUGAAAAAGGAUGCCAAACUCUCCUA | |
| | GACACCAGCAUAC | |
| R18150 | ACAGCUUAUUUGGAAGCUGAAAUGUGAGGUUUAUAACA | 602 |
| | CUCACAAGAAUCCUGAAAAAGGAUGCCAAACCAGAGUG | |
| | ACCACCGGGAAAU | |
| R18151 | ACAGCUUAUUUGGAAGCUGAAAUGUGAGGUUUAUAACA | 603 |
| | CUCACAAGAAUCCUGAAAAAGGAUGCCAAACUAUCUCC | |
| | UAGACACCAGCAU | |
| R18152 | ACAGCUUAUUUGGAAGCUGAAAUGUGAGGUUUAUAACA | 604 |
| | CUCACAAGAAUCCUGAAAAAGGAUGCCAAACACCACCG | |
| | GGAAAUCGAGGGC | |
| R18153 | ACAGCUUAUUUGGAAGCUGAAAUGUGAGGUUUAUAACA | 605 |
| | CUCACAAGAAUCCUGAAAAAGGAUGCCAAACGAGGUGU | |
| | AUCUCCUAGACAC | |
| R18154 | ACAGCUUAUUUGGAAGCUGAAAUGUGAGGUUUAUAACA | 606 |
| | CUCACAAGAAUCCUGAAAAAGGAUGCCAAACCCCGAGG | |
| | AGGACGGGACCCG | |
| R18123 | ACAGCUUAUUUGGAAGCUGAAAUGUGAGGUUUAUAACA | 607 |
| | CUCACAAGAAUCCUGAAAAAGGAUGCCAAACCCCUUCC | |
| | CUUGGCAGUUGAG | |
| R18124 | ACAGCUUAUUUGGAAGCUGAAAUGUGAGGUUUAUAACA | 608 |
| | CUCACAAGAAUCCUGAAAAAGGAUGCCAAACGCAGUUG | |
| | AGCACGCGCAGGC | |
| R18125 | ACAGCUUAUUUGGAAGCUGAAAUGUGAGGUUUAUAACA | 609 |
| | CUCACAAGAAUCCUGAAAAAGGAUGCCAAACACCGUGC | |
| | CCUUCCCUUGGCA | |
| R18126 | ACAGCUUAUUUGGAAGCUGAAAUGUGAGGUUUAUAACA | 610 |
| | CUCACAAGAAUCCUGAAAAAGGAUGCCAAACGCCACGC | |
| | CGGCAUCCCGGCC | |
| R18127 | ACAGCUUAUUUGGAAGCUGAAAUGUGAGGUUUAUAACA | 611 |
| | CUCACAAGAAUCCUGAAAAAGGAUGCCAAACACCACCC | |
| | CUGCCAGGUGGGU | |
| R18128 | ACAGCUUAUUUGGAAGCUGAAAUGUGAGGUUUAUAACA | 612 |
| | CUCACAAGAAUCCUGAAAAAGGAUGCCAAACGCAGGGG | |
| | UGGUCAGCGGCCG | |
| R18129 | ACAGCUUAUUUGGAAGCUGAAAUGUGAGGUUUAUAACA | 613 |
| | CUCACAAGAAUCCUGAAAAAGGAUGCCAAACCUCAACU | |
| | GCCAAGGGAAGGG | |
| R18130 | ACAGCUUAUUUGGAAGCUGAAAUGUGAGGUUUAUAACA | 614 |
| | CUCACAAGAAUCCUGAAAAAGGAUGCCAAACGUCAGCG | |
| | GCCGGGAUGCCGG | |
| R18131 | ACAGCUUAUUUGGAAGCUGAAAUGUGAGGUUUAUAACA | 615 |
| | CUCACAAGAAUCCUGAAAAAGGAUGCCAAACCGCGUGC | |
| | UCAACUGCCAAGG | |
| R18132 | ACAGCUUAUUUGGAAGCUGAAAUGUGAGGUUUAUAACA | 616 |
| | CUCACAAGAAUCCUGAAAAAGGAUGCCAAACGCACCCA | |
| | CCUGGCAGGGGUG | |
| R18103 | ACAGCUUAUUUGGAAGCUGAAAUGUGAGGUUUAUAACA | 617 |
| | CUCACAAGAAUCCUGAAAAAGGAUGCCAAACCCGGCAG | |
| | CGGUGACCAGCAC | |
| R18104 | ACAGCUUAUUUGGAAGCUGAAAUGUGAGGUUUAUAACA | 618 |
| | CUCACAAGAAUCCUGAAAAAGGAUGCCAAACGCUUUUC | |
| | CGAAUAAACUCCA | |
| R18105 | ACAGCUUAUUUGGAAGCUGAAAUGUGAGGUUUAUAACA | 619 |
| | CUCACAAGAAUCCUGAAAAAGGAUGCCAAACUACCCAC | |
| | CCGCCAGGGGCAG | |
| R18106 | ACAGCUUAUUUGGAAGCUGAAAUGUGAGGUUUAUAACA | 620 |
| | CUCACAAGAAUCCUGAAAAAGGAUGCCAAACGACCAGC | |
| | UGGCUUUUCCGAA | |
| R18107 | ACAGCUUAUUUGGAAGCUGAAAUGUGAGGUUUAUAACA | 621 |
| | CUCACAAGAAUCCUGAAAAAGGAUGCCAAACCCCACCC | |
| | GCCAGGGGCAGCA | |
| R18108 | ACAGCUUAUUUGGAAGCUGAAAUGUGAGGUUUAUAACA | 622 |
| | CUCACAAGAAUCCUGAAAAAGGAUGCCAAACGGGAGUA | |
| | GAGGCAGGCAUCG | |
| R18109 | ACAGCUUAUUUGGAAGCUGAAAUGUGAGGUUUAUAACA | 623 |
| | CUCACAAGAAUCCUGAAAAAGGAUGCCAAACACCAGCA | |
| | CGACCCCAGCCCU | |
| R18110 | ACAGCUUAUUUGGAAGCUGAAAUGUGAGGUUUAUAACA | 624 |
| | CUCACAAGAAUCCUGAAAAAGGAUGCCAAACGAGGCAG | |
| | GCAUCGUCCCGGA | |
| R18111 | ACAGCUUAUUUGGAAGCUGAAAUGUGAGGUUUAUAACA | 625 |
| | CUCACAAGAAUCCUGAAAAAGGAUGCCAAACCCCCUGG | |
| | CGGGUGGGUACAG | |
| R18112 | ACAGCUUAUUUGGAAGCUGAAAUGUGAGGUUUAUAACA | 626 |
| | CUCACAAGAAUCCUGAAAAAGGAUGCCAAACGGGUCGU | |
| | GCUGGUCACCGCU | |
| R18113 | ACAGCUUAUUUGGAAGCUGAAAUGUGAGGUUUAUAACA | 627 |
| | CUCACAAGAAUCCUGAAAAAGGAUGCCAAACCUGGUCA | |
| | CCGCUGCCGGCAA | |
| R18114 | ACAGCUUAUUUGGAAGCUGAAAUGUGAGGUUUAUAACA | 628 |
| | CUCACAAGAAUCCUGAAAAAGGAUGCCAAACGUCACCG | |
| | CUGCCGGCAACUU | |
| R18115 | ACAGCUUAUUUGGAAGCUGAAAUGUGAGGUUUAUAACA | 629 |
| | CUCACAAGAAUCCUGAAAAAGGAUGCCAAACCUGCCCC | |
| | UGGCGGGUGGGUA | |
| R18116 | ACAGCUUAUUUGGAAGCUGAAAUGUGAGGUUUAUAACA | 630 |
| | CUCACAAGAAUCCUGAAAAAGGAUGCCAAACGAGUUUA | |
| | UUCGGAAAAGCCA | |
| R18117 | ACAGCUUAUUUGGAAGCUGAAAUGUGAGGUUUAUAACA | 631 |
| | CUCACAAGAAUCCUGAAAAAGGAUGCCAAACGCGAGGG | |
| | CUGGGGUCGUGCU | |
| R18118 | ACAGCUUAUUUGGAAGCUGAAAUGUGAGGUUUAUAACA | 632 |
| | CUCACAAGAAUCCUGAAAAAGGAUGCCAAACCUGCUGC | |
| | CCCUGGCGGGUGG | |
| R18119 | ACAGCUUAUUUGGAAGCUGAAAUGUGAGGUUUAUAACA | 633 |
| | CUCACAAGAAUCCUGAAAAAGGAUGCCAAACUUCGGAA | |
| | AAGCCAGCUGGUC | |
| R18120 | ACAGCUUAUUUGGAAGCUGAAAUGUGAGGUUUAUAACA | 634 |
| | CUCACAAGAAUCCUGAAAAAGGAUGCCAAACCCUGCCU | |
| | CUACUCCCCAGCC | |
| R18121 | ACAGCUUAUUUGGAAGCUGAAAUGUGAGGUUUAUAACA | 635 |
| | CUCACAAGAAUCCUGAAAAAGGAUGCCAAACCCAGCGC | |
| | CUGGCGAGGGCUG | |
| R18122 | ACAGCUUAUUUGGAAGCUGAAAUGUGAGGUUUAUAACA | 636 |
| | CUCACAAGAAUCCUGAAAAAGGAUGCCAAACCCGGCAA | |
| | CUUCCGGGACGAU | |
| R18082 | ACAGCUUAUUUGGAAGCUGAAAUGUGAGGUUUAUAACA | 637 |
| | CUCACAAGAAUCCUGAAAAAGGAUGCCAAACUCCUCCC | |
| | CUGGGGCAAAGAG | |
| R18083 | ACAGCUUAUUUGGAAGCUGAAAUGUGAGGUUUAUAACA | 638 |
| | CUCACAAGAAUCCUGAAAAAGGAUGCCAAACGAGGCAC | |
| | CAAUGAUGUCCUC | |
| R18084 | ACAGCUUAUUUGGAAGCUGAAAUGUGAGGUUUAUAACA | 639 |
| | CUCACAAGAAUCCUGAAAAAGGAUGCCAAACGGCAUUG | |
| | GUGGCCCCAACUG | |
| R18085 | ACAGCUUAUUUGGAAGCUGAAAUGUGAGGUUUAUAACA | 640 |
| | CUCACAAGAAUCCUGAAAAAGGAUGCCAAACAUGUCCU | |
| | CCCCUGGGGCAAA | |
| R18086 | ACAGCUUAUUUGGAAGCUGAAAUGUGAGGUUUAUAACA | 641 |
| | CUCACAAGAAUCCUGAAAAAGGAUGCCAAACACACAAA | |
| | GCAGGUGCUGCAG | |
| R18087 | ACAGCUUAUUUGGAAGCUGAAAUGUGAGGUUUAUAACA | 642 |
| | CUCACAAGAAUCCUGAAAAAGGAUGCCAAACGUGGCCC | |
| | CAACUGUGAUGAC | |
| R18088 | ACAGCUUAUUUGGAAGCUGAAAUGUGAGGUUUAUAACA | 643 |
| | CUCACAAGAAUCCUGAAAAAGGAUGCCAAACCUGCAGU | |
| | CGCUGGAGGCACC | |
| R18089 | ACAGCUUAUUUGGAAGCUGAAAUGUGAGGUUUAUAACA | 644 |
| | CUCACAAGAAUCCUGAAAAAGGAUGCCAAACCAGUCGC | |
| | UGGAGGCACCAAU | |
| R18090 | ACAGCUUAUUUGGAAGCUGAAAUGUGAGGUUUAUAACA | 645 |
| | CUCACAAGAAUCCUGAAAAAGGAUGCCAAACGUCCCCA | |
| | AAGUCCCCAGGGU | |
| R18091 | ACAGCUUAUUUGGAAGCUGAAAUGUGAGGUUUAUAACA | 646 |
| | CUCACAAGAAUCCUGAAAAAGGAUGCCAAACGGGCAAA | |
| | GAGGUCCACACAG | |
| R18092 | ACAGCUUAUUUGGAAGCUGAAAUGUGAGGUUUAUAACA | 647 |
| | CUCACAAGAAUCCUGAAAAAGGAUGCCAAACGUGCCUC | |
| | CAGCGACUGCAGC | |
| R18093 | ACAGCUUAUUUGGAAGCUGAAAUGUGAGGUUUAUAACA | 648 |
| | CUCACAAGAAUCCUGAAAAAGGAUGCCAAACCCUCCAG | |
| | CGACUGCAGCACC | |
| R18094 | ACAGCUUAUUUGGAAGCUGAAAUGUGAGGUUUAUAACA | 649 |
| | CUCACAAGAAUCCUGAAAAAGGAUGCCAAACGCCGCUG | |
| | UGUGGACCUCUUU | |
| R18095 | ACAGCUUAUUUGGAAGCUGAAAUGUGAGGUUUAUAACA | 650 |
| | CUCACAAGAAUCCUGAAAAAGGAUGCCAAACGACCUCU | |
| | UUGCCCCAGGGGA | |
| R18096 | ACAGCUUAUUUGGAAGCUGAAAUGUGAGGUUUAUAACA | 651 |
| | CUCACAAGAAUCCUGAAAAAGGAUGCCAAACACCCUGG | |
| | GGACUUUGGGGAC | |
| R18097 | ACAGCUUAUUUGGAAGCUGAAAUGUGAGGUUUAUAACA | 652 |
| | CUCACAAGAAUCCUGAAAAAGGAUGCCAAACUGGACCU | |
| | CUUUGCCCCAGGG | |
| R18098 | ACAGCUUAUUUGGAAGCUGAAAUGUGAGGUUUAUAACA | 653 |
| | CUCACAAGAAUCCUGAAAAAGGAUGCCAAACCAGCACC | |
| | UGCUUUGUGUCAC | |
| R18099 | ACAGCUUAUUUGGAAGCUGAAAUGUGAGGUUUAUAACA | 654 |
| | CUCACAAGAAUCCUGAAAAAGGAUGCCAAACGGGACCA | |
| | ACUUUGGCCGCUG | |
| R18100 | ACAGCUUAUUUGGAAGCUGAAAUGUGAGGUUUAUAACA | 655 |
| | CUCACAAGAAUCCUGAAAAAGGAUGCCAAACGGGACUU | |
| | UGGGGACCAACUU | |
| R18101 | ACAGCUUAUUUGGAAGCUGAAAUGUGAGGUUUAUAACA | 656 |
| | CUCACAAGAAUCCUGAAAAAGGAUGCCAAACUGUGGAC | |
| | CUCUUUGCCCCAG | |
| R18102 | ACAGCUUAUUUGGAAGCUGAAAUGUGAGGUUUAUAACA | 657 |
| | CUCACAAGAAUCCUGAAAAAGGAUGCCAAACCCCCAGG | |
| | GGAGGACAUCAUU | |
| R18066 | ACAGCUUAUUUGGAAGCUGAAAUGUGAGGUUUAUAACA | 658 |
| | CUCACAAGAAUCCUGAAAAAGGAUGCCAAACGAUCAGU | |
| | CUCUGCCUCAACU | |
| R18067 | ACAGCUUAUUUGGAAGCUGAAAUGUGAGGUUUAUAACA | 659 |
| | CUCACAAGAAUCCUGAAAAAGGAUGCCAAACGCAGAGA | |
| | AGUGGAUCAGUCU | |
| R18068 | ACAGCUUAUUUGGAAGCUGAAAUGUGAGGUUUAUAACA | 660 |
| | CUCACAAGAAUCCUGAAAAAGGAUGCCAAACAGCUCCG | |
| | GCUCGGCAGACAG | |
| R18069 | ACAGCUUAUUUGGAAGCUGAAAUGUGAGGUUUAUAACA | 661 |
| | CUCACAAGAAUCCUGAAAAAGGAUGCCAAACGUCCUCA | |
| | GGGAACCAGGCCU | |
| R18070 | ACAGCUUAUUUGGAAGCUGAAAUGUGAGGUUUAUAACA | 662 |
| | CUCACAAGAAUCCUGAAAAAGGAUGCCAAACAUGACAU | |
| | CUUUGGCAGAGAA | |
| R18071 | ACAGCUUAUUUGGAAGCUGAAAUGUGAGGUUUAUAACA | 663 |
| | CUCACAAGAAUCCUGAAAAAGGAUGCCAAACACAUCUU | |
| | UGGCAGAGAAGUG | |
| R18072 | ACAGCUUAUUUGGAAGCUGAAAUGUGAGGUUUAUAACA | 664 |
| | CUCACAAGAAUCCUGAAAAAGGAUGCCAAACCUGUCUG | |
| | CCGAGCCGGAGCU | |
| R18073 | ACAGCUUAUUUGGAAGCUGAAAUGUGAGGUUUAUAACA | 665 |
| | CUCACAAGAAUCCUGAAAAAGGAUGCCAAACCAGCCAU | |
| | GAUGCUGUCUGCC | |
| R18074 | ACAGCUUAUUUGGAAGCUGAAAUGUGAGGUUUAUAACA | 666 |
| | CUCACAAGAAUCCUGAAAAAGGAUGCCAAACAUCCACU | |
| | UCUCUGCCAAAGA | |
| R18075 | ACAGCUUAUUUGGAAGCUGAAAUGUGAGGUUUAUAACA | 667 |
| | CUCACAAGAAUCCUGAAAAAGGAUGCCAAACAGGCCUG | |
| | GUUCCCUGAGGAC | |
| R18076 | ACAGCUUAUUUGGAAGCUGAAAUGUGAGGUUUAUAACA | 668 |
| | CUCACAAGAAUCCUGAAAAAGGAUGCCAAACAUGCUGU | |
| | CUGCCGAGCCGGA | |
| R18077 | ACAGCUUAUUUGGAAGCUGAAAUGUGAGGUUUAUAACA | 669 |
| | CUCACAAGAAUCCUGAAAAAGGAUGCCAAACAGGCAGA | |
| | GACUGAUCCACUU | |
| R18078 | ACAGCUUAUUUGGAAGCUGAAAUGUGAGGUUUAUAACA | 670 |
| | CUCACAAGAAUCCUGAAAAAGGAUGCCAAACCCAAAGA | |
| | UGUCAUCAAUGAG | |
| R18079 | ACAGCUUAUUUGGAAGCUGAAAUGUGAGGUUUAUAACA | 671 |
| | CUCACAAGAAUCCUGAAAAAGGAUGCCAAACUCUGCCG | |
| | AGCCGGAGCUCAC | |
| R18080 | ACAGCUUAUUUGGAAGCUGAAAUGUGAGGUUUAUAACA | 672 |
| | CUCACAAGAAUCCUGAAAAAGGAUGCCAAACGCCGAGU | |
| | UGAGGCAGAGACU | |
| R18081 | ACAGCUUAUUUGGAAGCUGAAAUGUGAGGUUUAUAACA | 673 |
| | CUCACAAGAAUCCUGAAAAAGGAUGCCAAACUCAUCAA | |
| | UGAGGCCUGGUUC | |
| R18051 | ACAGCUUAUUUGGAAGCUGAAAUGUGAGGUUUAUAACA | 674 |
| | CUCACAAGAAUCCUGAAAAAGGAUGCCAAACUGGCCAU | |
| | CCGUGUAGGCCCC | |
| R18052 | ACAGCUUAUUUGGAAGCUGAAAUGUGAGGUUUAUAACA | 675 |
| | CUCACAAGAAUCCUGAAAAAGGAUGCCAAACGAGCAGC | |
| | UCAGCAGCUCCUC | |
| R18053 | ACAGCUUAUUUGGAAGCUGAAAUGUGAGGUUUAUAACA | 676 |
| | CUCACAAGAAUCCUGAAAAAGGAUGCCAAACCGCUCGC | |
| | CCCGCCGCUUCCC | |
| R18054 | ACAGCUUAUUUGGAAGCUGAAAUGUGAGGUUUAUAACA | 677 |
| | CUCACAAGAAUCCUGAAAAAGGAUGCCAAACGAGAAAC | |
| | UGGAGCAGCUCAG | |
| R18055 | ACAGCUUAUUUGGAAGCUGAAAUGUGAGGUUUAUAACA | 678 |
| | CUCACAAGAAUCCUGAAAAAGGAUGCCAAACGCCAUCC | |
| | GUGUAGGCCCCGA | |
| R18056 | ACAGCUUAUUUGGAAGCUGAAAUGUGAGGUUUAUAACA | 679 |
| | CUCACAAGAAUCCUGAAAAAGGAUGCCAAACUAGGCCC | |
| | CGAGUGUGCUGAC | |
| R18057 | ACAGCUUAUUUGGAAGCUGAAAUGUGAGGUUUAUAACA | 680 |
| | CUCACAAGAAUCCUGAAAAAGGAUGCCAAACGGCCCCG | |
| | AGUGUGCUGACCA | |
| R18058 | ACAGCUUAUUUGGAAGCUGAAAUGUGAGGUUUAUAACA | 681 |
| | CUCACAAGAAUCCUGAAAAAGGAUGCCAAACGGAAGCG | |
| | GCGGGGCGAGCGC | |
| R18059 | ACAGCUUAUUUGGAAGCUGAAAUGUGAGGUUUAUAACA | 682 |
| | CUCACAAGAAUCCUGAAAAAGGAUGCCAAACCUGAGCU | |
| | GCUCCAGUUUCUC | |
| R18060 | ACAGCUUAUUUGGAAGCUGAAAUGUGAGGUUUAUAACA | 683 |
| | CUCACAAGAAUCCUGAAAAAGGAUGCCAAACUGGUCAG | |
| | CACACUCGGGGCC | |
| R18061 | ACAGCUUAUUUGGAAGCUGAAAUGUGAGGUUUAUAACA | 684 |
| | CUCACAAGAAUCCUGAAAAAGGAUGCCAAACCUCCAGU | |
| | UUCUCCAGGAGUG | |
| R18062 | ACAGCUUAUUUGGAAGCUGAAAUGUGAGGUUUAUAACA | 685 |
| | CUCACAAGAAUCCUGAAAAAGGAUGCCAAACGCAGCUG | |
| | UUUUGCAGGACUG | |
| R18063 | ACAGCUUAUUUGGAAGCUGAAAUGUGAGGUUUAUAACA | 686 |
| | CUCACAAGAAUCCUGAAAAAGGAUGCCAAACAGGAGCU | |
| | GCUGAGCUGCUCC | |
| R18064 | ACAGCUUAUUUGGAAGCUGAAAUGUGAGGUUUAUAACA | 687 |
| | CUCACAAGAAUCCUGAAAAAGGAUGCCAAACAGCUGCU | |
| | CCAGUUUCUCCAG | |
| R18065 | ACAGCUUAUUUGGAAGCUGAAAUGUGAGGUUUAUAACA | 688 |
| | CUCACAAGAAUCCUGAAAAAGGAUGCCAAACGUCAGCA | |
| | CACUCGGGGCCUA | |
| R18034 | ACAGCUUAUUUGGAAGCUGAAAUGUGAGGUUUAUAACA | 689 |
| | CUCACAAGAAUCCUGAAAAAGGAUGCCAAACGAGCUGU | |
| | GUGGACGCUGCAG | |
| R18035 | ACAGCUUAUUUGGAAGCUGAAAUGUGAGGUUUAUAACA | 690 |
| | CUCACAAGAAUCCUGAAAAAGGAUGCCAAACGCAGUGG | |
| | ACACGGGUCCCCA | |
| R18036 | ACAGCUUAUUUGGAAGCUGAAAUGUGAGGUUUAUAACA | 691 |
| | CUCACAAGAAUCCUGAAAAAGGAUGCCAAACGCGUAGA | |
| | CACCCUCACCCCC | |
| R18037 | ACAGCUUAUUUGGAAGCUGAAAUGUGAGGUUUAUAACA | 692 |
| | CUCACAAGAAUCCUGAAAAAGGAUGCCAAACGACACCC | |
| | UCACCCCCAAAAG | |
| R18038 | ACAGCUUAUUUGGAAGCUGAAAUGUGAGGUUUAUAACA | 693 |
| | CUCACAAGAAUCCUGAAAAAGGAUGCCAAACGACACGG | |
| | GUCCCCAUGCUGG | |
| R18039 | ACAGCUUAUUUGGAAGCUGAAAUGUGAGGUUUAUAACA | 694 |
| | CUCACAAGAAUCCUGAAAAAGGAUGCCAAACGGGUAGC | |
| | AGGCAGCACCUGG | |
| R18040 | ACAGCUUAUUUGGAAGCUGAAAUGUGAGGUUUAUAACA | 695 |
| | CUCACAAGAAUCCUGAAAAAGGAUGCCAAACGCAGGCA | |
| | GCACCUGGCAAUG | |
| R18041 | ACAGCUUAUUUGGAAGCUGAAAUGUGAGGUUUAUAACA | 696 |
| | CUCACAAGAAUCCUGAAAAAGGAUGCCAAACGUGGAGC | |
| | UGUGUGGACGCUG | |
| R18042 | ACAGCUUAUUUGGAAGCUGAAAUGUGAGGUUUAUAACA | 697 |
| | CUCACAAGAAUCCUGAAAAAGGAUGCCAAACGCAAUGG | |
| | CGUAGACACCCUC | |
| R18043 | ACAGCUUAUUUGGAAGCUGAAAUGUGAGGUUUAUAACA | 698 |
| | CUCACAAGAAUCCUGAAAAAGGAUGCCAAACCCAGGUG | |
| | CUGCCUGCUACCC | |
| R18044 | ACAGCUUAUUUGGAAGCUGAAAUGUGAGGUUUAUAACA | 699 |
| | CUCACAAGAAUCCUGAAAAAGGAUGCCAAACGGGGUGA | |
| | GGGUGUCUACGCC | |
| R18045 | ACAGCUUAUUUGGAAGCUGAAAUGUGAGGUUUAUAACA | 700 |
| | CUCACAAGAAUCCUGAAAAAGGAUGCCAAACCGCCAUU | |
| | GCCAGGUGCUGCC | |
| R18046 | ACAGCUUAUUUGGAAGCUGAAAUGUGAGGUUUAUAACA | 701 |
| | CUCACAAGAAUCCUGAAAAAGGAUGCCAAACAGGGUGU | |
| | CUACGCCAUUGCC | |
| R18047 | ACAGCUUAUUUGGAAGCUGAAAUGUGAGGUUUAUAACA | 702 |
| | CUCACAAGAAUCCUGAAAAAGGAUGCCAAACUCUACGC | |
| | CAUUGCCAGGUGC | |
| R18048 | ACAGCUUAUUUGGAAGCUGAAAUGUGAGGUUUAUAACA | 703 |
| | CUCACAAGAAUCCUGAAAAAGGAUGCCAAACCCGGGCC | |
| | CACAACGCUUUUG | |
| R18049 | ACAGCUUAUUUGGAAGCUGAAAUGUGAGGUUUAUAACA | 704 |
| | CUCACAAGAAUCCUGAAAAAGGAUGCCAAACCAGCGUC | |
| | CACACAGCUCCAC | |
| R18050 | ACAGCUUAUUUGGAAGCUGAAAUGUGAGGUUUAUAACA | 705 |
| | CUCACAAGAAUCCUGAAAAAGGAUGCCAAACGGGACCC | |
| | GUGUCCACUGCCA | |
| R18008 | ACAGCUUAUUUGGAAGCUGAAAUGUGAGGUUUAUAACA | 706 |
| | CUCACAAGAAUCCUGAAAAAGGAUGCCAAACUGGGUGC | |
| | CAAGGUCCUCCAC | |
| R18009 | ACAGCUUAUUUGGAAGCUGAAAUGUGAGGUUUAUAACA | 707 |
| | CUCACAAGAAUCCUGAAAAAGGAUGCCAAACCCAAGGU | |
| | CCUCCACCUCCCA | |
| R18010 | ACAGCUUAUUUGGAAGCUGAAAUGUGAGGUUUAUAACA | 708 |
| | CUCACAAGAAUCCUGAAAAAGGAUGCCAAACACUUUGC | |
| | AUUCCAGACCUGG | |
| R18011 | ACAGCUUAUUUGGAAGCUGAAAUGUGAGGUUUAUAACA | 709 |
| | CUCACAAGAAUCCUGAAAAAGGAUGCCAAACGCAGCAG | |
| | GAAGCGUGGAUGC | |
| R18012 | ACAGCUUAUUUGGAAGCUGAAAUGUGAGGUUUAUAACA | 710 |
| | CUCACAAGAAUCCUGAAAAAGGAUGCCAAACGGCUGAC | |
| | CUCGUGGCCUCAG | |
| R18013 | ACAGCUUAUUUGGAAGCUGAAAUGUGAGGUUUAUAACA | 711 |
| | CUCACAAGAAUCCUGAAAAAGGAUGCCAAACGCCUCAG | |
| | CACAGGCGGCUUG | |
| R18014 | ACAGCUUAUUUGGAAGCUGAAAUGUGAGGUUUAUAACA | 712 |
| | CUCACAAGAAUCCUGAAAAAGGAUGCCAAACACCUCGU | |
| | GGCCUCAGCACAG | |
| R18015 | ACAGCUUAUUUGGAAGCUGAAAUGUGAGGUUUAUAACA | 713 |
| | CUCACAAGAAUCCUGAAAAAGGAUGCCAAACCUCCUUG | |
| | ACUUUGCAUUCCA | |
| R18016 | ACAGCUUAUUUGGAAGCUGAAAUGUGAGGUUUAUAACA | 714 |
| | CUCACAAGAAUCCUGAAAAAGGAUGCCAAACCAUUCCA | |
| | GACCUGGGGCAUG | |
| R18017 | ACAGCUUAUUUGGAAGCUGAAAUGUGAGGUUUAUAACA | 715 |
| | CUCACAAGAAUCCUGAAAAAGGAUGCCAAACGGUGCCA | |
| | AGGUCCUCCACCU | |
| R18018 | ACAGCUUAUUUGGAAGCUGAAAUGUGAGGUUUAUAACA | 716 |
| | CUCACAAGAAUCCUGAAAAAGGAUGCCAAACGUUGGGC | |
| | UGACCUCGUGGCC | |
| R18019 | ACAGCUUAUUUGGAAGCUGAAAUGUGAGGUUUAUAACA | 717 |
| | CUCACAAGAAUCCUGAAAAAGGAUGCCAAACGGGCAUG | |
| | GCAGCAGGAAGCG | |
| R18020 | ACAGCUUAUUUGGAAGCUGAAAUGUGAGGUUUAUAACA | 718 |
| | CUCACAAGAAUCCUGAAAAAGGAUGCCAAACAGGGGCC | |
| | GGGAUUCCAUGCU | |
| R18021 | ACAGCUUAUUUGGAAGCUGAAAUGUGAGGUUUAUAACA | 719 |
| | CUCACAAGAAUCCUGAAAAAGGAUGCCAAACUGCUGAG | |
| | GCCACGAGGUCAG | |
| R18022 | ACAGCUUAUUUGGAAGCUGAAAUGUGAGGUUUAUAACA | 720 |
| | CUCACAAGAAUCCUGAAAAAGGAUGCCAAACGCACCCA | |
| | CAAGCCGCCUGUG | |
| R18023 | ACAGCUUAUUUGGAAGCUGAAAUGUGAGGUUUAUAACA | 721 |
| | CUCACAAGAAUCCUGAAAAAGGAUGCCAAACCCCCAGG | |
| | UCUGGAAUGCAAA | |
| R18024 | ACAGCUUAUUUGGAAGCUGAAAUGUGAGGUUUAUAACA | 722 |
| | CUCACAAGAAUCCUGAAAAAGGAUGCCAAACGAGGACC | |
| | UUGGCACCCACAA | |
| R18025 | ACAGCUUAUUUGGAAGCUGAAAUGUGAGGUUUAUAACA | 723 |
| | CUCACAAGAAUCCUGAAAAAGGAUGCCAAACCUGAGGC | |
| | CACGAGGUCAGCC | |
| R18026 | ACAGCUUAUUUGGAAGCUGAAAUGUGAGGUUUAUAACA | 724 |
| | CUCACAAGAAUCCUGAAAAAGGAUGCCAAACGAAUGCA | |
| | AAGUCAAGGAGCA | |
| R18027 | ACAGCUUAUUUGGAAGCUGAAAUGUGAGGUUUAUAACA | 725 |
| | CUCACAAGAAUCCUGAAAAAGGAUGCCAAACCCAUGCC | |
| | CCAGGUCUGGAAU | |
| R18028 | ACAGCUUAUUUGGAAGCUGAAAUGUGAGGUUUAUAACA | 726 |
| | CUCACAAGAAUCCUGAAAAAGGAUGCCAAACCUGCCAU | |
| | GCCCCAGGUCUGG | |
| R18029 | ACAGCUUAUUUGGAAGCUGAAAUGUGAGGUUUAUAACA | 727 |
| | CUCACAAGAAUCCUGAAAAAGGAUGCCAAACGGAGGUG | |
| | GAGGACCUUGGCA | |
| R18030 | ACAGCUUAUUUGGAAGCUGAAAUGUGAGGUUUAUAACA | 728 |
| | CUCACAAGAAUCCUGAAAAAGGAUGCCAAACAGGCCAC | |
| | GAGGUCAGCCCAA | |
| R18031 | ACAGCUUAUUUGGAAGCUGAAAUGUGAGGUUUAUAACA | 729 |
| | CUCACAAGAAUCCUGAAAAAGGAUGCCAAACCAAAGUC | |
| | AAGGAGCAUGGAA | |
| R18032 | ACAGCUUAUUUGGAAGCUGAAAUGUGAGGUUUAUAACA | 730 |
| | CUCACAAGAAUCCUGAAAAAGGAUGCCAAACCAGCUCC | |
| | CACUGGGAGGUGG | |
| R18033 | ACAGCUUAUUUGGAAGCUGAAAUGUGAGGUUUAUAACA | 731 |
| | CUCACAAGAAUCCUGAAAAAGGAUGCCAAACGAAUCCC | |
| | GGCCCCUCAGGAG | |
| R17967 | ACAGCUUAUUUGGAAGCUGAAAUGUGAGGUUUAUAACA | 732 |
| | CUCACAAGAAUCCUGAAAAAGGAUGCCAAACGCUGUAA | |
| | AAAGGCAACAGAG | |
| R17968 | ACAGCUUAUUUGGAAGCUGAAAUGUGAGGUUUAUAACA | 733 |
| | CUCACAAGAAUCCUGAAAAAGGAUGCCAAACCAAAAGC | |
| | AAAACAGGUCUAG | |
| R17969 | ACAGCUUAUUUGGAAGCUGAAAUGUGAGGUUUAUAACA | 734 |
| | CUCACAAGAAUCCUGAAAAAGGAUGCCAAACAAUGUCU | |
| | GCUUGCUUGGGUG | |
| R17970 | ACAGCUUAUUUGGAAGCUGAAAUGUGAGGUUUAUAACA | 735 |
| | CUCACAAGAAUCCUGAAAAAGGAUGCCAAACAAAAUGC | |
| | UACAAAACCCAGA | |
| R17971 | ACAGCUUAUUUGGAAGCUGAAAUGUGAGGUUUAUAACA | 736 |
| | CUCACAAGAAUCCUGAAAAAGGAUGCCAAACCUUGCUU | |
| | GGGUGGGGCUGGU | |
| R17972 | ACAGCUUAUUUGGAAGCUGAAAUGUGAGGUUUAUAACA | 737 |
| | CUCACAAGAAUCCUGAAAAAGGAUGCCAAACCUACAAA | |
| | ACCCAGAAUAAAU | |
| R17973 | ACAGCUUAUUUGGAAGCUGAAAUGUGAGGUUUAUAACA | 738 |
| | CUCACAAGAAUCCUGAAAAAGGAUGCCAAACUCUGCUU | |
| | GCUUGGGUGGGGC | |
| R17974 | ACAGCUUAUUUGGAAGCUGAAAUGUGAGGUUUAUAACA | 739 |
| | CUCACAAGAAUCCUGAAAAAGGAUGCCAAACGGUGGGG | |
| | CUGGUGCUCAAGG | |
| R17975 | ACAGCUUAUUUGGAAGCUGAAAUGUGAGGUUUAUAACA | 740 |
| | CUCACAAGAAUCCUGAAAAAGGAUGCCAAACAAAAGGC | |
| | AACAGAGAGGACA | |
| R17976 | ACAGCUUAUUUGGAAGCUGAAAUGUGAGGUUUAUAACA | 741 |
| | CUCACAAGAAUCCUGAAAAAGGAUGCCAAACAUAAAAA | |
| | UGCUACAAAACCC | |
| R17977 | ACAGCUUAUUUGGAAGCUGAAAUGUGAGGUUUAUAACA | 742 |
| | CUCACAAGAAUCCUGAAAAAGGAUGCCAAACGUCUGUG | |
| | UUCCCCUUCCCAG | |
| R17978 | ACAGCUUAUUUGGAAGCUGAAAUGUGAGGUUUAUAACA | 743 |
| | CUCACAAGAAUCCUGAAAAAGGAUGCCAAACUUCCCCU | |
| | UCCCAGCCUCACU | |
| R17979 | ACAGCUUAUUUGGAAGCUGAAAUGUGAGGUUUAUAACA | 744 |
| | CUCACAAGAAUCCUGAAAAAGGAUGCCAAACUAAAAAG | |
| | GCAACAGAGAGGA | |
| R17980 | ACAGCUUAUUUGGAAGCUGAAAUGUGAGGUUUAUAACA | 745 |
| | CUCACAAGAAUCCUGAAAAAGGAUGCCAAACUCUUCAA | |
| | GUUACAAAAGCAA | |
| R17981 | ACAGCUUAUUUGGAAGCUGAAAUGUGAGGUUUAUAACA | 746 |
| | CUCACAAGAAUCCUGAAAAAGGAUGCCAAACGUGCUCA | |
| | AGGAGGGACAGUU | |
| R17982 | ACAGCUUAUUUGGAAGCUGAAAUGUGAGGUUUAUAACA | 747 |
| | CUCACAAGAAUCCUGAAAAAGGAUGCCAAACGGGCUGG | |
| | UGCUCAAGGAGGG | |
| R17983 | ACAGCUUAUUUGGAAGCUGAAAUGUGAGGUUUAUAACA | 748 |
| | CUCACAAGAAUCCUGAAAAAGGAUGCCAAACCUUGGGU | |
| | GGGGCUGGUGCUC | |
| R17984 | ACAGCUUAUUUGGAAGCUGAAAUGUGAGGUUUAUAACA | 749 |
| | CUCACAAGAAUCCUGAAAAAGGAUGCCAAACCAAAACC | |
| | CAGAAUAAAUAUC | |
| R17985 | ACAGCUUAUUUGGAAGCUGAAAUGUGAGGUUUAUAACA | 750 |
| | CUCACAAGAAUCCUGAAAAAGGAUGCCAAACUGUUCCC | |
| | CUUCCCAGCCUCA | |
| R17986 | ACAGCUUAUUUGGAAGCUGAAAUGUGAGGUUUAUAACA | 751 |
| | CUCACAAGAAUCCUGAAAAAGGAUGCCAAACGACCUGU | |
| | UUUGCUUUUGUAA | |
| R17987 | ACAGCUUAUUUGGAAGCUGAAAUGUGAGGUUUAUAACA | 752 |
| | CUCACAAGAAUCCUGAAAAAGGAUGCCAAACCUUUUGU | |
| | AACUUGAAGAUAU | |
| R17988 | ACAGCUUAUUUGGAAGCUGAAAUGUGAGGUUUAUAACA | 753 |
| | CUCACAAGAAUCCUGAAAAAGGAUGCCAAACUCCUCUC | |
| | UGUUGCCUUUUUA | |
| R17989 | ACAGCUUAUUUGGAAGCUGAAAUGUGAGGUUUAUAACA | 754 |
| | CUCACAAGAAUCCUGAAAAAGGAUGCCAAACGGUCUGU | |
| | CCUCUCUGUUGCC | |
| R17990 | ACAGCUUAUUUGGAAGCUGAAAUGUGAGGUUUAUAACA | 755 |
| | CUCACAAGAAUCCUGAAAAAGGAUGCCAAACUUCUGGG | |
| | UUUUGUAGCAUUU | |
| R17991 | ACAGCUUAUUUGGAAGCUGAAAUGUGAGGUUUAUAACA | 756 |
| | CUCACAAGAAUCCUGAAAAAGGAUGCCAAACUCCCUCC | |
| | UUGAGCACCAGCC | |
| R17992 | ACAGCUUAUUUGGAAGCUGAAAUGUGAGGUUUAUAACA | 757 |
| | CUCACAAGAAUCCUGAAAAAGGAUGCCAAACAAGAUAU | |
| | UUAUUCUGGGUUU | |
| R17993 | ACAGCUUAUUUGGAAGCUGAAAUGUGAGGUUUAUAACA | 758 |
| | CUCACAAGAAUCCUGAAAAAGGAUGCCAAACUUUAUUC | |
| | UGGGUUUUGUAGC | |
| R17994 | ACAGCUUAUUUGGAAGCUGAAAUGUGAGGUUUAUAACA | 759 |
| | CUCACAAGAAUCCUGAAAAAGGAUGCCAAACACUUGAA | |
| | GAUAUUUAUUCUG | |
| R17995 | ACAGCUUAUUUGGAAGCUGAAAUGUGAGGUUUAUAACA | 760 |
| | CUCACAAGAAUCCUGAAAAAGGAUGCCAAACAGGCUGG | |
| | GAAGGGGAACACA | |
| R17996 | ACAGCUUAUUUGGAAGCUGAAAUGUGAGGUUUAUAACA | 761 |
| | CUCACAAGAAUCCUGAAAAAGGAUGCCAAACUCUUUUG | |
| | GGUCUGUCCUCUC | |
| R17997 | ACAGCUUAUUUGGAAGCUGAAAUGUGAGGUUUAUAACA | 762 |
| | CUCACAAGAAUCCUGAAAAAGGAUGCCAAACUUUUGCU | |
| | UUUGUAACUUGAA | |
| R17998 | ACAGCUUAUUUGGAAGCUGAAAUGUGAGGUUUAUAACA | 763 |
| | CUCACAAGAAUCCUGAAAAAGGAUGCCAAACGGUUUUG | |
| | UAGCAUUUUUAUU | |
| R17999 | ACAGCUUAUUUGGAAGCUGAAAUGUGAGGUUUAUAACA | 764 |
| | CUCACAAGAAUCCUGAAAAAGGAUGCCAAACAGCACCA | |
| | GCCCCACCCAAGC | |
| R18000 | ACAGCUUAUUUGGAAGCUGAAAUGUGAGGUUUAUAACA | 765 |
| | CUCACAAGAAUCCUGAAAAAGGAUGCCAAACGGAAGGG | |
| | GAACACAGACCAG | |
| R18001 | ACAGCUUAUUUGGAAGCUGAAAUGUGAGGUUUAUAACA | 766 |
| | CUCACAAGAAUCCUGAAAAAGGAUGCCAAACCCGGCUC | |
| | CGGCAGCAGAUGG | |
| R18002 | ACAGCUUAUUUGGAAGCUGAAAUGUGAGGUUUAUAACA | 767 |
| | CUCACAAGAAUCCUGAAAAAGGAUGCCAAACGGAGGUC | |
| | CCAGGGAGGGCAC | |
| R18003 | ACAGCUUAUUUGGAAGCUGAAAUGUGAGGUUUAUAACA | 768 |
| | CUCACAAGAAUCCUGAAAAAGGAUGCCAAACGGAUGGG | |
| | GCUGUCACUGGAG | |
| R18004 | ACAGCUUAUUUGGAAGCUGAAAUGUGAGGUUUAUAACA | 769 |
| | CUCACAAGAAUCCUGAAAAAGGAUGCCAAACCAGUGCC | |
| | CUCCCUGGGACCU | |
| R18005 | ACAGCUUAUUUGGAAGCUGAAAUGUGAGGUUUAUAACA | 770 |
| | CUCACAAGAAUCCUGAAAAAGGAUGCCAAACCCAUCUG | |
| | CUGCCGGAGCCGG | |
| R18006 | ACAGCUUAUUUGGAAGCUGAAAUGUGAGGUUUAUAACA | 771 |
| | CUCACAAGAAUCCUGAAAAAGGAUGCCAAACACAGCCC | |
| | CAUCCCAGGAUGG | |
| R18007 | ACAGCUUAUUUGGAAGCUGAAAUGUGAGGUUUAUAACA | 772 |
| | CUCACAAGAAUCCUGAAAAAGGAUGCCAAACCUGCCGG | |
| | AGCCGGCACCUGG | |
| n/a | ACAGCUUAUUUGGAAGCUGAAAUGUGAGGUUUAUAACA | 829 |
| | CUCACAAGAAUCCUGAAAAAGGAUGCCAAACUAGAACC | |
| | UUGAUGACAUAGC | |
| PL34563 | ACAGCUUAUUUGGAAGCUGAAAUGUGAGGUUUAUAACA | 2027 |
| | CUCACAAGAAUCCUGAAAAAGGAUGCCAAACCUUACUU | |
| | UAAGUGAAGUUAC | |
| PL34564 | ACAGCUUAUUUGGAAGCUGAAAUGUGAGGUUUAUAACA | 2028 |
| | CUCACAAGAAUCCUGAAAAAGGAUGCCAAACUUUUCUA | |
| | CUUACUUUAAGUG | |
| PL34565 | ACAGCUUAUUUGGAAGCUGAAAUGUGAGGUUUAUAACA | 2029 |
| | CUCACAAGAAUCCUGAAAAAGGAUGCCAAACUCCAGAC | |
| | UUUUGUAGAAAAA | |
| PL34566 | ACAGCUUAUUUGGAAGCUGAAAUGUGAGGUUUAUAACA | 2030 |
| | CUCACAAGAAUCCUGAAAAAGGAUGCCAAACAAAUACU | |
| | GACUUACCUGAUU | |
| PL34567 | ACAGCUUAUUUGGAAGCUGAAAUGUGAGGUUUAUAACA | 2031 |
| | CUCACAAGAAUCCUGAAAAAGGAUGCCAAACUCAGCUC | |
| | AGAAGGACUAGUA | |
| PL34568 | ACAGCUUAUUUGGAAGCUGAAAUGUGAGGUUUAUAACA | 2032 |
| | CUCACAAGAAUCCUGAAAAAGGAUGCCAAACUCUUACC | |
| | AUCAUGUUUUACA | |
| PL34569 | ACAGCUUAUUUGGAAGCUGAAAUGUGAGGUUUAUAACA | 2033 |
| | CUCACAAGAAUCCUGAAAAAGGAUGCCAAACUUGAUUC | |
| | UAGGCAUUCCUGC | |
| PL34570 | ACAGCUUAUUUGGAAGCUGAAAUGUGAGGUUUAUAACA | 2034 |
| | CUCACAAGAAUCCUGAAAAAGGAUGCCAAACUUCAGGU | |
| | AGUCCAUGGACAU | |
| PL34571 | ACAGCUUAUUUGGAAGCUGAAAUGUGAGGUUUAUAACA | 2035 |
| | CUCACAAGAAUCCUGAAAAAGGAUGCCAAACGUCCCCU | |
| | UACCAUCAAGCCU | |
| PL34572 | ACAGCUUAUUUGGAAGCUGAAAUGUGAGGUUUAUAACA | 2036 |
| | CUCACAAGAAUCCUGAAAAAGGAUGCCAAACAAACUUU | |
| | UCUUUUCAGGAGA | |
| PL34573 | ACAGCUUAUUUGGAAGCUGAAAUGUGAGGUUUAUAACA | 2037 |
| | CUCACAAGAAUCCUGAAAAAGGAUGCCAAACUCAGAAA | |
| | AAGAUACCUGAAU | |
| PL34574 | ACAGCUUAUUUGGAAGCUGAAAUGUGAGGUUUAUAACA | 2038 |
| | CUCACAAGAAUCCUGAAAAAGGAUGCCAAACUCUCCUU | |
| | UAGGAGGCUGGUG | |
| PL34575 | ACAGCUUAUUUGGAAGCUGAAAUGUGAGGUUUAUAACA | 2039 |
| | CUCACAAGAAUCCUGAAAAAGGAUGCCAAACUCUUGUU | |
| | UUUCUACAAAAGU | |
| PL34576 | ACAGCUUAUUUGGAAGCUGAAAUGUGAGGUUUAUAACA | 2040 |
| | CUCACAAGAAUCCUGAAAAAGGAUGCCAAACAAAGAAA | |
| | UAGAAAAUCAGGU | |
| PL34577 | ACAGCUUAUUUGGAAGCUGAAAUGUGAGGUUUAUAACA | 2041 |
| | CUCACAAGAAUCCUGAAAAAGGAUGCCAAACAAUACUA | |
| | GUCCUUCUGAGCU | |
| PL34578 | ACAGCUUAUUUGGAAGCUGAAAUGUGAGGUUUAUAACA | 2042 |
| | CUCACAAGAAUCCUGAAAAAGGAUGCCAAACAGAAAUG | |
| | UAAAACAUGAUGG | |
| PL34579 | ACAGCUUAUUUGGAAGCUGAAAUGUGAGGUUUAUAACA | 2043 |
| | CUCACAAGAAUCCUGAAAAAGGAUGCCAAACCAUUCAG | |
| | CAGGAAUGCCUAG | |
| PL34580 | ACAGCUUAUUUGGAAGCUGAAAUGUGAGGUUUAUAACA | 2044 |
| | CUCACAAGAAUCCUGAAAAAGGAUGCCAAACGUGGUAC | |
| | AUUCAGCAGGAAU | |
| PL34581 | ACAGCUUAUUUGGAAGCUGAAAUGUGAGGUUUAUAACA | 2045 |
| | CUCACAAGAAUCCUGAAAAAGGAUGCCAAACAAUUAAU | |
| | GUCCAUGGACUAC | |
| PL34582 | ACAGCUUAUUUGGAAGCUGAAAUGUGAGGUUUAUAACA | 2046 |
| | CUCACAAGAAUCCUGAAAAAGGAUGCCAAACGUUUUGG | |
| | GAGGCUUGAUGGU | |
| PL34583 | ACAGCUUAUUUGGAAGCUGAAAUGUGAGGUUUAUAACA | 2047 |
| | CUCACAAGAAUCCUGAAAAAGGAUGCCAAACGGCCCAA | |
| | CCAAAAUUCUCCU | |
| PL34584 | ACAGCUUAUUUGGAAGCUGAAAUGUGAGGUUUAUAACA | 2048 |
| | CUCACAAGAAUCCUGAAAAAGGAUGCCAAACUCCAGAG | |
| | GGUUAUUCAGGUA | |
| PL34585 | ACAGCUUAUUUGGAAGCUGAAAUGUGAGGUUUAUAACA | 2049 |
| | CUCACAAGAAUCCUGAAAAAGGAUGCCAAACCUUACGG | |
| | GCAGAGGCCAGGA | |
| PL34586 | ACAGCUUAUUUGGAAGCUGAAAUGUGAGGUUUAUAACA | 2050 |
| | CUCACAAGAAUCCUGAAAAAGGAUGCCAAACCUCUUUC | |
| | CUCAGGAGCUUCA | |
| PL34587 | ACAGCUUAUUUGGAAGCUGAAAUGUGAGGUUUAUAACA | 2051 |
| | CUCACAAGAAUCCUGAAAAAGGAUGCCAAACAUUUAGG | |
| | GGCUGGGUGACCG | |
| PL34588 | ACAGCUUAUUUGGAAGCUGAAAUGUGAGGUUUAUAACA | 2052 |
| | CUCACAAGAAUCCUGAAAAAGGAUGCCAAACACUGAUU | |
| | UAGGGGCUGGGUG | |

TABLE 13
Exemplary Guide Nucleic Acids Targeting
ANGPTL3 for CasM.265466 Effector Proteins

| | | SEQ |
|---|---|---|
| Guide | Guide sequence (shown as RNA), | ID |
| ID | 5′-3′ | NO: |
| PL34532 | ACAGCUUAUUUGGAAGCUGAAAUGUGAGGU | 2053 |
| | UUAUAACACUCACAAGAAUCCUGAAAAAGG | |
| | AUGCCAAACCUUCCCCUGACUGAUUUAGG | |
| PL34533 | ACAGCUUAUUUGGAAGCUGAAAUGUGAGGU | 2054 |
| | UUAUAACACUCACAAGAAUCCUGAAAAAGG | |
| | AUGCCAAACGAGGCAGCUGCUCCAGGUAA | |
| PL34534 | ACAGCUUAUUUGGAAGCUGAAAUGUGAGGU | 2055 |
| | UUAUAACACUCACAAGAAUCCUGAAAAAGG | |
| | AUGCCAAACCAUGGCACCUCUGUUCCUGC | |
| PL34535 | ACAGCUUAUUUGGAAGCUGAAAUGUGAGGU | 2056 |
| | UUAUAACACUCACAAGAAUCCUGAAAAAGG | |
| | AUGCCAAACGCGCUCCUGGCCUCUGCCCG | |
| PL34536 | ACAGCUUAUUUGGAAGCUGAAAUGUGAGGU | 2057 |
| | UUAUAACACUCACAAGAAUCCUGAAAAAGG | |
| | AUGCCAAACAAGCCAUCGGUCACCCAGCC | |
| PL34537 | ACAGCUUAUUUGGAAGCUGAAAUGUGAGGU | 2058 |
| | UUAUAACACUCACAAGAAUCCUGAAAAAGG | |
| | AUGCCAAACCACCCGCACCUUGGCGCAGC | |
| PL34538 | ACAGCUUAUUUGGAAGCUGAAAUGUGAGGU | 2059 |
| | UUAUAACACUCACAAGAAUCCUGAAAAAGG | |
| | AUGCCAAACGGGCCAGGAUCCGUGGAGGU | |
| PL34539 | ACAGCUUAUUUGGAAGCUGAAAUGUGAGGU | 2060 |
| | UUAUAACACUCACAAGAAUCCUGAAAAAGG | |
| | AUGCCAAACGCUCACCAGCUCCAGCAGGU | |
| PL34540 | ACAGCUUAUUUGGAAGCUGAAAUGUGAGGU | 2061 |
| | UUAUAACACUCACAAGAAUCCUGAAAAAGG | |
| | AUGCCAAACGCUUCUGCAGGCCUUGAAGU | |
| PL34541 | ACAGCUUAUUUGGAAGCUGAAAUGUGAGGU | 2062 |
| | UUAUAACACUCACAAGAAUCCUGAAAAAGG | |
| | AUGCCAAACGGGGUCUUACCGGGGGGCUG | |
| PL34542 | ACAGCUUAUUUGGAAGCUGAAAUGUGAGGU | 2063 |
| | UUAUAACACUCACAAGAAUCCUGAAAAAGG | |
| | AUGCCAAACGAAAGACGGAGGCAGCCUGG | |
| PL34543 | ACAGCUUAUUUGGAAGCUGAAAUGUGAGGU | 2064 |
| | UUAUAACACUCACAAGAAUCCUGAAAAAGG | |
| | AUGCCAAACCUUACCUGUCUGUGGAAGCG | |
| PL34544 | ACAGCUUAUUUGGAAGCUGAAAUGUGAGGU | 2065 |
| | UUAUAACACUCACAAGAAUCCUGAAAAAGG | |
| | AUGCCAAACUUCGUCGAGCAGGCCAGCAA | |
| PL34545 | ACAGCUUAUUUGGAAGCUGAAAUGUGAGGU | 2066 |
| | UUAUAACACUCACAAGAAUCCUGAAAAAGG | |
| | AUGCCAAACGGGCCAUCACUUACCUAUGA | |
| PL34546 | ACAGCUUAUUUGGAAGCUGAAAUGUGAGGU | 2067 |
| | UUAUAACACUCACAAGAAUCCUGAAAAAGG | |
| | AUGCCAAACUUCCUCCCAGGCCUGGAGUU | |
| PL34547 | ACAGCUUAUUUGGAAGCUGAAAUGUGAGGU | 2068 |
| | UUAUAACACUCACAAGAAUCCUGAAAAAGG | |
| | AUGCCAAACAUGACCUGGAAAGGUGAGGA | |
| PL34548 | ACAGCUUAUUUGGAAGCUGAAAUGUGAGGU | 2069 |
| | UUAUAACACUCACAAGAAUCCUGAAAAAGG | |
| | AUGCCAAACCACCAGGCAUUGCAGCCAUG | |
| PL34549 | ACAGCUUAUUUGGAAGCUGAAAUGUGAGGU | 2070 |
| | UUAUAACACUCACAAGAAUCCUGAAAAAGG | |
| | AUGCCAAACCUUACCUGCCCCAUGGGUGC | |
| PL34550 | ACAGCUUAUUUGGAAGCUGAAAUGUGAGGU | 2071 |
| | UUAUAACACUCACAAGAAUCCUGAAAAAGG | |
| | AUGCCAAACCAGUCACCUCCAUGCGCUCG | |
| PL34551 | ACAGCUUAUUUGGAAGCUGAAAUGUGAGGU | 2072 |
| | UUAUAACACUCACAAGAAUCCUGAAAAAGG | |
| | AUGCCAAACACUCUAAGGCCCAAGGGGGC | |
| PL34552 | ACAGCUUAUUUGGAAGCUGAAAUGUGAGGU | 2073 |
| | UUAUAACACUCACAAGAAUCCUGAAAAAGG | |
| | AUGCCAAACCCCCAGGCUGCAGCUCCCAC | |
| PL34553 | ACAGCUUAUUUGGAAGCUGAAAUGUGAGGU | 2074 |
| | UUAUAACACUCACAAGAAUCCUGAAAAAGG | |
| | AUGCCAAACGCAGGUGACCGUGGCCUGCG | |

In some embodiments, guide nucleic acids comprise a portion or all of a sequence as set forth in any one of TABLES 1, 7, or 8. In some embodiments, a guide nucleic acid comprises at least 9, at least 10, at least 11, at least 12 contiguous nucleotides of a sequence selected from any one of SEQ ID NOs: 1-31, 38-43, 67-78, 207, 491, 804-805, 815-816, 830-999, and 1400-1569. In some embodiments, the guide nucleic acid comprises at least 15, at least 20, at least 25, at least 30, or at least 35 contiguous nucleotides of a sequence selected from any one of SEQ ID NOs: 1-31, 38-43, 67-78, 207, 491, 804-805, 815-816, 830-999, and 1400-1569.

In some embodiments, guide nucleic acids comprise a portion or all of a sequence as set forth in any one of TABLES 3, 7, or 9. In some embodiments, a guide nucleic acid comprises at least 9, at least 10, at least 11, at least 12 contiguous nucleotides of a sequence selected from any one of SEQ ID NOs: 16, 38-43, 79-202, 208, 492-493, 799-803, 809-814, and 820. In some embodiments, the guide nucleic acid comprises at least 15, at least 20, at least 25, at least 30, or at least 35 contiguous nucleotides of a sequence selected from any one of SEQ ID NOs: 16, 38-43, 79-202, 208, 492-493, 799-803, 809-814, and 820.

In some embodiments, guide nucleic acids comprise a portion or all of a sequence as set forth in any one of TABLES 5, 7, or 10. In some embodiments, a guide nucleic acid comprises at least 9, at least 10, at least 11, at least 12 contiguous nucleotides of a sequence selected from any one of SEQ ID NOs: 16, 38-43, 806-808, and 817-819. In some embodiments, the guide nucleic acid comprises at least 15, at least 20, at least 25, at least 30, or at least 35 contiguous nucleotides of a sequence selected from any one of SEQ ID NOs: 16, 38-43, 806-808, and 817-819.

In some embodiments, compositions disclosed herein comprises a spacer sequence that is at least 70%, at least 75%, at least 80%, at least 85%, at least 90%, at least 95%, or 100% identical to any one of the sequences as set forth in TABLES 1, 3, and 5, and comprising a repeat sequence that is at least 70%, at least 75%, at least 80%, at least 85%, at least 90%, at least 95%, or 100% identical to a sequence selected from any one of SEQ ID NOs: 16 or 38-43.

In some embodiments, compositions disclosed herein comprises a guide nucleic acid sequence that is at least 70%, at least 75%, at least 80%, at least 85%, at least 90%, at least 95%, or 100% identical to any one of the sequences as set forth in TABLES 8-10.

In some embodiments, guide nucleic acids comprise a portion or all of a sequence as set forth in any one of TABLES 2, 7, or 11. In some embodiments, a guide nucleic acid comprises at least 40, at least 45, at least 50, at least 55, at least 60, at least 65, at least 70, at least 75, at least 80, at least 85, at least 86, at least 87, at least 88, or at least 89 contiguous nucleotides of a sequence selected from any one of SEQ ID NOs: 44, 209-299, 488-490, 494-584, 823-828, 1000-1399, 1570-1969, 2018-2026, and 2075-2089.

In some embodiments, guide nucleic acids comprise a portion or all of a sequence as set forth in any one of TABLES 4, 7, or 12. In some embodiments, a guide nucleic acid comprises at least 40, at least 45, at least 50, at least 55, at least 60, at least 65, at least 70, at least 75, at least 80, at least 85, at least 86, at least 87, at least 88, or at least 89 contiguous nucleotides of a sequence selected from any one of SEQ ID NOs: 44, 300-490, 585-772, 822, 829, 1970-1995, and 2027-2052.

In some embodiments, guide nucleic acids comprise a portion or all of a sequence as set forth in any one of TABLES 6, 7, or 13. In some embodiments, a guide nucleic acid at least 40, at least 45, at least 50, at least 55, at least 60, at least 65, at least 70, at least 75, at least 80, at least 85, at least 86, at least 87, at least 88, or at least 89 contiguous nucleotides of any one of SEQ ID NOs: 44, 488-490, 1996-2017, and 2053-2074.

In some embodiments, compositions, systems, and methods described herein comprise a disclosed herein comprises a spacer sequence that is at least 70%, at least 75%, at least 80%, at least 85%, at least 90%, at least 92%, at least 95%, at least 97%, at least 99%, or 100% identical to any one of the sequences as set forth in TABLES 2, 4, and 6, and comprising a repeat sequence that is at least 65%, at least 70%, at least 75%, at least 80%, at least 85%, at least 90%, at least 95%, or 100% identical to SEQ ID NO: 488.

In some embodiments, compositions disclosed herein comprises a guide nucleic acid sequence that is at least 70%, at least 75%, at least 80%, at least 85%, at least 90%, at least 95%, or 100% identical to any one of the sequences as set forth in TABLE 11-13.

In some embodiments, the sequences in any one of TABLES 1-13 and SEQ ID NOs: 44, and 489-490 can be modified.

In some embodiments, the modification includes at least one phosphorothioate (PS) linkage. In some embodiments, the modification includes at least one 2′-O-Methyl oligonucleotide (OMe). In some embodiments, the modification includes at least one locked nucleic acid (LNA). In some embodiments, the modification includes at least one Phosphorodiamidate morpholino oligonucleotide (PMO). In some embodiments, the modification includes at least one or more peptide nucleic acid (PNA). In some embodiments, the first 3 and last 3 amino acids are 0-Me modified, and the first 3 and last 2 linkages are phosphorothioate linkages. In some embodiments, the sequence is modified mN*mN*mN* . . . NNNmN*mN*mN where m is 2′-O-Me modified sugar moiety and the * denotes a PS linkage.

### Nucleic Acid Linkers

In some embodiments, a guide nucleic acid for use with compositions, systems, and methods described herein comprises one or more linkers, or a nucleic acid encoding one or more linkers. In some embodiments, the guide nucleic acid comprises at least one, at least two, at least three, at least four, at least five, at least six, at least seven, at least eight, at least nine, or at least ten linkers. In some embodiments, the guide nucleic acid comprises one, two, three, four, five, six, seven, eight, nine, or ten linkers. In some embodiments, the guide nucleic acid comprises two or more linkers. In some embodiments, at least two or more linkers are the same. In some embodiments, at least two or more linkers are not same.

In some embodiments, a linker comprises one to ten, one to seven, one to five, one to three, two to ten, two to eight, two to six, two to four, three to ten, three to seven, three to five, four to ten, four to eight, four to six, five to ten, five to seven, six to ten, six to eight, seven to ten, or eight to ten linked nucleotides. In some embodiments, the linker comprises one, two, three, four, five, six, seven, eight, nine, or ten linked nucleotides. In some embodiments, a linker comprises a nucleotide sequence of 5′-GAAA-3′ (SEQ ID NO: 44).

In some embodiments, a guide nucleic acid comprises one or more linkers connecting one or more repeat sequences. In some embodiments, the guide nucleic acid comprises one or more linkers connecting one or more repeat sequences and one or more spacer sequences. In some embodiments, the guide nucleic acid comprises at least two repeat sequences connected by a linker.

### 4. Effector Proteins

In some embodiments, compositions provided herein comprise one or more effector proteins or a nucleic acid encoding the same. In some embodiments, compositions and systems described herein comprise an effector protein that is similar to a naturally occurring effector protein. The effector protein may lack a portion of the naturally occurring effector protein. The effector protein may comprise a mutation relative to the naturally-occurring effector protein, wherein the mutation is not found in nature.

An effector protein may be brought into proximity of a target nucleic acid in the presence of a guide nucleic acid. The ability of an effector protein to modify a target nucleic acid may be dependent upon the effector protein being bound to a guide nucleic acid and the guide nucleic acid being hybridized to a target nucleic acid. An effector protein may also recognize a protospacer adjacent motif (PAM) sequence present in the target nucleic acid, which may direct the modification activity of the effector protein.

In some embodiments, the effector protein is a programmable nuclease (e.g., a CRISPR-associated (Cas) protein) that modifies a target sequence in a target nucleic acid. In some embodiments, the effector protein is a programmable nuclease that modifies a region of the nucleic acid that is near, but not within, to the target sequence. Effector proteins may cleave nucleic acids, including single stranded RNA (ssRNA), double stranded DNA (dsDNA), and single-stranded DNA (ssDNA). Effector proteins may provide cis cleavage activity, trans cleavage activity, nickase activity, or a combination thereof.

An effector protein may function as a single protein that is capable of binding to a guide nucleic acid and modifying a target nucleic acid. Alternatively, an effector protein may function as part of a multiprotein complex, including, for example, a complex having two or more effector proteins, including two or more of the same effector proteins (e.g., a dimer or a multimer). An effector protein, when functioning in a multiprotein complex, may have only one functional activity (e.g., binding to a guide nucleic acid), while other effector proteins present in the multiprotein complex are capable of another functional activity (e.g., modifying a target nucleic acid).

In some embodiments, the effector protein is a Type V Cas protein. In some embodiments, the effector protein is CasPhi.12 or a variant thereof. In some embodiments, the effector protein is CasM.265466 or a variant thereof. A CasPhi.12 is around half of the size of Cas9, and CasM.265466 is around one third of the size of Cas9. The smaller sizes of CasPhi.12 and CasM.265466 make them ideal to be packaged together with their corresponding guide RNAs into a single AAV vector, thus overcoming the drawbacks of dual AAV vector systems.

TABLE 15 provides illustrative amino acid sequences of effector proteins. In some embodiments, the amino acid sequence of an effector protein is at least 65%, at least 70%, at least 75%, at least 80%, at least 85%, at least 90%, at least 95%, at least 97%, or at least 98%, at least 99%, or 100% identical to the sequence as set forth in TABLE 15. In some embodiments, an effector protein comprises an amino acid sequence that is at least 65%, at least 70%, at least 75%, at least 80%, at least 85%, at least 90%, at least 95%, at least 97%, or at least 98%, at least 99%, or 100% identical to any one of the sequences as set forth in TABLE 15. In some embodiments, the effector protein consists of an amino acid sequence selected from the sequences as set forth in TABLE 15.

In some embodiments, compositions, systems, and methods comprise an effector protein or uses thereof, wherein the amino acid sequence of the effector protein comprises at least about 200, at least about 220, at least about 240, at least about 260, at least about 280, at least about 300, at least about 320, at least about 340, at least about 360, at least about 380, at least about 400, at least about 420, at least about 440, at least about 460, at least about 480, at least about 500, at least about 520, at least about 540, at least about 560, at least about 580, at least about 600, at least about 620, at least about 640, at least about 660, at least about 680, or at least about 700 contiguous amino acids of a sequence in TABLE 15.

In some embodiments, the effector protein may also comprise at least one additional amino acid relative to the naturally-occurring or wild type effector protein. For example, the effector protein may comprise an addition of a nuclear localization signal relative to the natural occurring effector protein. In some embodiments, compositions and systems described herein may comprise a nuclear localization signal (NLS). In some embodiments, the effector protein is linked to a nuclear localization signal. In some embodiments, compositions and systems described herein may comprise a NLS sequence that is adjacent to the N terminal of the effector protein or that is adjacent to the C terminal of the effector protein, or both. In some embodiments, a nuclear localization signal can comprise a sequence of -N-MAPKKKRKVGIHGVPAA-C (SEQ ID NO: 36). In some embodiments, a nuclear localization signal can comprise a sequence of -N-KRPAATKKAGQAKKKK-C (SEQ ID NO: 37). In certain embodiments, the nucleotide sequence encoding the effector protein is codon optimized (e.g., for expression in a eukaryotic cell) relative to the naturally occurring sequence.

TABLE 14 provides exemplary nuclear localization sequences. In TABLE 14, X is any naturally occurring amino acid, and \hat{ }D/E is any naturally occurring amino acid except Asp or Glu

TABLE 14
Exemplary Nuclear Localization Sequences

| SEQ | | |
|---|---|---|
| ID NO: | Description | Sequence |
| 47 | NLS | PKKKRKVGIHGVPAA |
| 48 | NLS | KRPAATKKAGQAKKKK |
| N/A | NLS | KR(K/R)R |
| N/A | NLS | (P/R)XXKR(^{∧}DE)(K/R) |
| 49 | NLS | KRX(W/F/Y)XXAF |
| N/A | NLS | (R/P)XXKR(K/R)(^{∧}DE) |
| 50 | NLS | LGKR(K/R)(W/F/Y) |
| N/A | NLS | KRX₁₀K(K/R)(K/R) |
| N/A | NLS | K(K/R)RK |
| N/A | NLS | KRX₁₁K(K/R)(K/R) |
| N/A | NLS | KRX₁₂K(K/R)(K/R) |
| N/A | NLS | KRX₁₀K(K/R)X(K/R) |
| N/A | NLS | KRX₁₁K(K/R)X(K/R) |
| N/A | NLS | KRX₁₂K(K/R)X(K/R) |
| 51 | NLS | APKKKRKVGIHGVPAA |
| 52 | EEP | GLFXALLXLLXSLWXLLLXA |
| 53 | EEP | GLFHALLHLLHSLWHLLLHA |

An effector protein may function as a single protein that is capable of binding to a guide nucleic acid and modifying a target nucleic acid. Alternatively, an effector protein may function as part of a multiprotein complex, including, for example, a complex having two or more effector proteins, including two or more of the same effector proteins (e.g., a dimer or a multimer). An effector protein, when functioning in a multiprotein complex, may have only one functional activity (e.g., binding to a guide nucleic acid), while other effector proteins present in the multiprotein complex are capable of another functional activity (e.g., modifying a target nucleic acid).

TABLE 15
Exemplary Effector Proteins

| Effector | | SEQ |
|---|---|---|
| Protein | Amino Acid Sequence | ID NO: |
| CasPhi.12 | MIKPTVSQFLTPGFKLIRNHSRTAGLKLKNEGEEACKKFVR | 32 |
| | ENEIPKDECPNFQGGPAIANIIAKSREFTEWEIYQSSLAIQEVI | |
| | FTLPKDKLPEPILKEEWRAQWLSEHGLDTVPYKEAAGLNLI | |
| | IKNAVNTYKGVQVKVDNKNKNNLAKINRKNEIAKLNGEQ | |
| | EISFEEIKAFDDKGYLLQKPSPNKSIYCYQSVSPKPFITSKYH | |
| | NVNLPEEYIGYYRKSNEPIVSPYQFDRLRIPIGEPGYVPKWQ | |
| | YTFLSKKENKRRKLSKRIKNVSPILGIICIKKDWCVFDMRGL | |
| | LRTNHWKKYHKPTDSINDLFDYFTGDPVIDTKANVVRFRY | |
| | KMENGIVNYKPVREKKGKELLENICDQNGSCKLATVDVG | |
| | QNNPVAIGLFELKKVNGELTKTLISRHPTPIDFCNKITAYRE | |
| | RYDKLESSIKLDAIKQLTSEQKIEVDNYNNNFTPQNTKQIVC | |
| | SKLNINPNDLPWDKMISGTHFISEKAQVSNKSEIYFTSTDKG | |
| | KTKDVMKSDYKWFQDYKPKLSKEVRDALSDIEWRLRRES | |
| | LEFNKLSKSREQDARQLANWISSMCDVIGIENLVKKNNFFG | |
| | GSGKREPGWDNFYKPKKENRWWINAIHKALTELSQNKGK | |
| | RVILLPAMRTSITCPKCKYCDSKNRNGEKFNCLKCGIELNA | |
| | DIDVATENLATVAITAQSMPKPTCERSGDAKKPVRARKAK | |
| | APEFHDKLAPSYTVVLREAV | |
| CasPhi.12 with | PKKKRKVGIHGVPAAMIKPTVSQFLTPGFKLIRNHSRTAGL | 33 |
| exemplary | KLKNEGEEACKKFVRENEIPKDECPNFQGGPAIANIIAKSRE | |
| NLS (bold, | FTEWEIYQSSLAIQEVIFTLPKDKLPEPILKEEWRAQWLSEH | |
| italicized) at N | GLDTVPYKEAAGLNLIIKNAVNTYKGVQVKVDNKNKNNL | |
| terminus and C | AKINRKNEIAKLNGEQEISFEEIKAFDDKGYLLQKPSPNKSI | |
| terminus | YCYQSVSPKPFITSKYHNVNLPEEYIGYYRKSNEPIVSPYQF | |
| | DRLRIPIGEPGYVPKWQYTFLSKKENKRRKLSKRIKNVSPIL | |
| | GIICIKKDWCVFDMRGLLRTNHWKKYHKPTDSINDLFDYF | |
| | TGDPVIDTKANVVRFRYKMENGIVNYKPVREKKGKELLEN | |
| | ICDQNGSCKLATVDVGQNNPVAIGLFELKKVNGELTKTLIS | |
| | RHPTPIDFCNKITAYRERYDKLESSIKLDAIKQLTSEQKIEVD | |
| | NYNNNFTPQNTKQIVCSKLNINPNDLPWDKMISGTHFISEK | |
| | AQVSNKSEIYFTSTDKGKTKDVMKSDYKWFQDYKPKLSK | |
| | EVRDALSDIEWRLRRESLEFNKLSKSREQDARQLANWISSM | |
| | CDVIGIENLVKKNNFFGGSGKREPGWDNFYKPKKENRWWI | |
| | NAIHKALTELSQNKGKRVILLPAMRTSITCPKCKYCDSKNR | |
| | NGEKFNCLKCGIELNADIDVATENLATVAITAQSMPKPTCE | |
| | RSGDAKKPVRARKAKAPEFHDKLAPSYTVVLREAVKRPAA | |
| | TKKAGQAKKKK | |
| CasM.265466 | MSVLTRKVQLIPVGDKEERDRVYKYLRDGIEAQNRAMNL | 773 |
| | YMSGLYFAAINEASKEDRKELNQLYSRIATSSKGSAYTTDI | |
| | EFPTGLASTSTLSMAVRQDFTKSLKDGLMYGRVSLPTYRK | |
| | DNPLFVDVRFVALRGTKQKYNGLYHEYKSHTEFLDNLYSS | |
| | DLKVYIKFANDITFQVIFGNPRKSSALRSEFQNIFEEYYKVC | |
| | QSSIQFSGTKIILNMAMDIPDKEIELDEDVCVGVDLGIAIPAV | |
| | CALNKNRYSRVSIGSKEDFLRVRTKIRNQRKRLQTNLKSSN | |
| | GGHGRKKKMKPMDRFRDYEANWVQNYNHYVSRQVVDF | |
| | AVKNKAKYINLENLEGIRDDVKNEWLLSNWSYYQLQQYIT | |
| | YKAKTYGIEVRKINPYHTSQRCSCCGYEDAGNRPKKEKGQ | |
| | AYFKCLKCGEEMNADFNAARNIAMSTEFQSGKKTKKQKK | |
| | EQHENK | |
| 3x Flag- | MDYKDHDGDYKDHDIDYKDDDDKMAPKKKRKVGIHGVP | 774 |
| SV40NLS- | AAMSVLTRKVQLIPVGDKEERDRVYKYLRDGIEAQNRAM | |
| CasM.265466- | NLYMSGLYFAAINEASKEDRKELNQLYSRIATSSKGSAYTT | |
| nucleoplasmin | DIEFPTGLASTSTLSMAVRQDFTKSLKDGLMYGRVSLPTYR | |
| NLS | KDNPLFVDVRFVALRGTKQKYNGLYHEYKSHTEFLDNLYS | |
| (Bold and | SDLKVYIKFANDITFQVIFGNPRKSSALRSEFQNIFEEYYKV | |
| italicized text | CQSSIQFSGTKIILNMAMDIPDKEIELDEDVCVGVDLGIAIPA | |
| indicates the | VCALNKNRYSRVSIGSKEDFLRVRTKIRNQRKRLQTNLKSS | |
| NLS. | NGGHGRKKKMKPMDRFRDYEANWVQNYNHYVSRQVVD | |
| Underlined | FAVKNKAKYINLENLEGIRDDVKNEWLLSNWSYYQLQQYI | |
| text indicates a | TYKAKTYGIEVRKINPYHTSQRCSCCGYEDAGNRPKKEKG | |
| 3xFLAG tag) | QAYFKCLKCGEEMNADFNAARNIAMSTEFQSGKKTKKQK | |
| | KEQHENKKRPAATKKAGQAKKKK | |

In some embodiments, compositions, systems, and methods described herein comprise an effector protein or a nucleic acid encoding the effector protein, wherein the effector protein comprises one or more amino acid alterations relative to a sequence recited in TABLE 15. In some embodiments, an amino acid alteration comprises a deletion of an amino acid. In some embodiments, an amino acid alteration comprises an insertion of an amino acid. In some embodiments, an amino acid alteration comprises a conservative amino acid substitution. In some embodiments, an amino acid alteration comprises a non-conservative amino acid substitution. In some embodiments, one or more amino acid alterations comprises a combination of one or more conservative amino acid substitutions and one or more non-conservative amino acid substitutions. When describing a conservative amino acid substitution herein, reference is made to the replacement of one amino acid for another such that the replacement takes place within a family of amino acids that are related in their side chains. Conversely, when describing a non-conservative alteration (e.g., non-conservative substitution), reference is made to the replacement of one amino acid residue for another that does not have a related side chain. It is understood that genetically encoded amino acids can be divided into four families having related side chains: (1) acidic (negatively charged): Asp (D), Glu (E); (2) basic (positively charged): Lys (K)Arg (R), His (H); (3) non-polar (hydrophobic): Cys (C), Ala (A), Val (V), Leu (L), Ile (I), Pro (P), Phe (F), Met (M), Trp (W), Gly (G), Tyr (Y), with non-polar also being subdivided into: (i) strongly hydrophobic: Ala (A), Val (V), Leu (L), Ile (I), Met (M), Phe (F); and (ii) moderately hydrophobic: Gly (G), Pro (P), Cys (C), Tyr (Y), Trp (W); and (4) uncharged polar: Asn (N), Gln (Q), Ser (S), Thr (T). Amino acids may be related by aliphatic side chains: Gly (G), Ala (A), Val (V), Leu (L), Ile (I), Ser (S), Thr (T), with Ser (S) and Thr (T) optionally being grouped separately as aliphatic-hydroxyl. Amino acids may be related by aromatic side chains: Phe (F), Tyr (Y), Trp (W). Amino acids may be related by amide side chains: Asn (N), Gln (Q). Amino acids may be related by sulfur-containing side chains: Cys (C) and Met (M).

In some embodiments, effector proteins disclosed herein are engineered proteins. Engineered proteins are not identical to a naturally-occurring protein. Engineered proteins may provide enhanced nuclease or nickase activity as compared to a naturally occurring nuclease or nickase. SEQ ID NO: 34 is a non-limiting example of an engineered protein, wherein residue 26 has been modified to an arginine from a leucine at residue 26 of SEQ ID NO: 32.

An engineered protein may comprise a modified form of a wild-type counterpart protein (e.g., an effector protein). The modified form of the wild-type counterpart may comprise an amino acid change (e.g., deletion, insertion, or substitution) that reduces the nucleic acid-cleaving activity of the effector protein relative to the wild-type counterpart. For example, a nuclease domain (e.g., RuvC domain) of an effector protein may be deleted or mutated relative to a wild-type counterpart effector protein so that it is no longer functional or comprises reduced nuclease activity. The modified form of the effector protein may have less than 90%, less than 80%, less than 70%, less than 60%, less than 50%, less than 40%, less than 30%, less than 20%, less than 10%, less than 5%, or less than 1% of the nucleic acid-cleaving activity of the wild-type counterpart.

In some embodiments, effector proteins are engineered variants of CasM.265466 (SEQ ID NO: 773) and CasPhi.12 (SEQ ID NO: 32). Engineered variants of CasM.265466 (SEQ ID NO: 773) and CasPhi.12 (SEQ ID NO: 32) may comprise amino acid substitutions relative to SEQ ID NO: 773 and SEQ ID NO: 32, respectively.

In some embodiments, an effector protein comprises an amino acid sequence that is at least 65%, at least 70%, at least 75%, at least 80%, at least 85%, at least 90%, at least 95%, at least 97%, or at least 98%, at least 99%, or 100% identical to SEQ ID NO: 32 wherein the amino acid residue at position 26 is arginine (R). In some embodiments, an effector protein comprises an amino acid sequence that is at least 65%, at least 70%, at least 75%, at least 80%, at least 85%, at least 90%, at least 95%, at least 97%, or at least 98%, at least 99%, or 100% identical to SEQ ID NO: 32 wherein the amino acid residue at position 471 is threonine (T). In some embodiments, an effector protein comprises an amino acid sequence that is at least 65%, at least 70%, at least 75%, at least 80%, at least 85%, at least 90%, at least 95%, at least 97%, or at least 98%, at least 99%, or 100% identical to SEQ ID NO: 32 wherein the amino acid residue at position 26 is arginine (R) and the amino acid residue at position 471 is threonine (T).

In some embodiments, an effector protein comprises an amino acid sequence that is at least 65%, at least 70%, at least 75%, at least 80%, at least 85%, at least 90%, at least 95%, at least 97%, or at least 98%, or at least 99%, identical to SEQ ID NO: 773 wherein the amino acid residue at position 220 is arginine (R). In some embodiments, an effector protein comprises an amino acid sequence that is at least 65%, at least 70%, at least 75%, at least 80%, at least 85%, at least 90%, at least 95%, at least 97%, or at least 98%, at least 99%, or 100% identical to SEQ ID NO: 773 wherein the amino acid residue at position 220 is arginine (R) and the amino acid residue at position 335 is glutamine (Q).

Exemplary amino acid substitutions are described in TABLES 16-19. The amino acid substitutions in TABLE 16 and TABLE 17 may be combined. The amino acid substitutions in TABLE 16 and TABLE 17 may be combined with other amino acid alterations described herein. The amino acid substitutions in TABLE 18 and TABLE 19 may be combined. The amino acid substitutions in TABLE 18 and TABLE 19 may be combined with other amino acid alterations described herein.

In certain embodiments, compositions comprise an effector protein and a guide nucleic acid, wherein the effector protein comprises an amino acid sequence that is at least 65%, at least 70%, at least 75%, at least 80%, at least 85%, at least 90%, at least 95%, at least 98%, at least 99%, or 100% identical to any one of the sequences as set forth in TABLE 15. In certain embodiments, compositions comprise an effector protein and a guide nucleic acid, wherein the effector protein comprises an amino acid sequence that is at least 65%, at least 70%, at least 75%, at least 80%, at least 85%, at least 90%, at least 95%, at least 97%, or at least 98%, at least 99%, or 100% identical to any one of the sequences as set forth in TABLE 15, wherein the amino acid residue at position 220, relative to SEQ ID NO: 775, remains unchanged. In other words, the residue of the amino acid sequence that aligns with position 220 of SEQ ID NO: 775 is an arginine when the amino acid sequence is aligned with SEQ ID NO: 773 for maximum identity. In some embodiments, the amino acid sequence of the effector protein is at least 65%, at least 70%, at least 75%, at least 80%, at least 85%, at least 90%, at least 95%, at least 97%, or at least 98%, at least 99%, or 100% identical to any one of the sequences as set forth in TABLE 15. In some embodiments, the amino acid sequence of the effector protein is at least 65%, at least 70%, at least 75%, at least 80%, at least 85%, at least 90%, at least 95%, at least 97%, or at least 98%, at least 99%, or 100% identical to any one of the sequences as set forth in TABLE 15, wherein the amino acid residue at position 220, relative to SEQ ID NO: 773, remains unchanged.

In certain embodiments, compositions comprise an effector protein and a guide nucleic acid, wherein the effector protein comprises an amino acid sequence that is at least 65%, at least 70%, at least 75%, at least 80%, at least 85%, at least 90%, at least 95%, at least 98%, at least 99%, or 100% identical to any one of the sequences as set forth in TABLE 15. In certain embodiments, compositions comprise an effector protein and a guide nucleic acid, wherein the effector protein comprises an amino acid sequence that is at least 65%, at least 70%, at least 75%, at least 80%, at least 85%, at least 90%, at least 95%, at least 97%, or at least 98%, at least 99%, or 100% identical to any one of the sequences as set forth in TABLE 15, wherein the amino acid residue at position 26, relative to SEQ ID NO: 34, remains unchanged. In other words, the residue of the amino acid sequence that aligns with position 26 of SEQ ID NO: 32 is an arginine. In some embodiments, the amino acid sequence of the effector protein is at least 65%, at least 70%, at least 75%, at least 80%, at least 85%, at least 90%, at least 95%, at least 97%, or at least 98%, at least 99%, or 100% identical to any one of the sequences as set forth in TABLE 15. In some embodiments, the amino acid sequence of the effector protein is at least 65%, at least 70%, at least 75%, at least 80%, at least 85%, at least 90%, at least 95%, at least 97%, or at least 98%, at least 99%, or 100% identical to any one of the sequences as set forth in TABLE 15, wherein the amino acid residue at position 26, relative to SEQ ID NO: 34, remains unchanged.

In certain embodiments, the amino acid sequence of the effector protein is based on SEQ ID NO: 32 and is modified at position 26. In some embodiments, the modification at position 26 is from leucine to arginine (L26R). In some embodiments, the amino acid sequence of the effector protein is at least 95%, 96%, 97%, 98%, or 99% identical to SEQ ID NO: 34. In some embodiments, the amino acid sequence of the effector protein comprises or consists of SEQ ID NO: 34.

In certain embodiments, the amino acid sequence of the effector protein is based on SEQ ID NO: 32 and is modified at position 109. In some embodiments, the modification at position 109 is from glutamic acid to arginine (E109R). In some embodiments, the amino acid sequence of the effector protein is at least 95%, 96%, 97%, 98%, or 99% identical to SEQ ID NO: 54. In some embodiments, the amino acid sequence of the effector protein comprises or consists of SEQ ID NO: 54.

In certain embodiments, the amino acid sequence of the effector protein is based on SEQ ID NO: 32 and is modified at position 208. In some embodiments, the modification at position 208 is from histidine to arginine (H208R). In some embodiments, the amino acid sequence of the effector protein is at least 95%, 96%, 97%, 98%, or 99% identical to SEQ ID NO: 55. In some embodiments, the amino acid sequence of the effector protein comprises or consists of SEQ ID NO: 55.

In certain embodiments, the amino acid sequence of the effector protein is based on SEQ ID NO: 32 and is modified at position 184. In some embodiments, the modification at position 184 is from lysine to arginine (K184R). In some embodiments, the amino acid sequence of the effector protein is at least 95%, 96%, 97%, 98%, or 99% identical to SEQ ID NO: 56. In some embodiments, the amino acid sequence of the effector protein comprises or consists of SEQ ID NO: 56.

In certain embodiments, the amino acid sequence of the effector protein is based on SEQ ID NO: 32 and is modified at position 38. In some embodiments, the modification at position 38 is from lysine to arginine (K38R). In some embodiments, the amino acid sequence of the effector protein is at least 95%, 96%, 97%, 98%, or 99% identical to SEQ ID NO: 57. In some embodiments, the amino acid sequence of the effector protein comprises or consists of SEQ ID NO: 57.

In certain embodiments, the amino acid sequence of the effector protein is based on SEQ ID NO: 32 and is modified at position 182. In some embodiments, the modification at position 182 is from leucine to arginine (L182R). In some embodiments, the amino acid sequence of the effector protein is at least 95%, 96%, 97%, 98%, or 99% identical to SEQ ID NO: 58. In some embodiments, the amino acid sequence of the effector protein comprises or consists of SEQ ID NO: 58.

In certain embodiments, the amino acid sequence of the effector protein is based on SEQ ID NO: 32 and is modified at position 183. In some embodiments, the modification at position 183 is from glutamine to arginine (Q183R). In some embodiments, the amino acid sequence of the effector protein is at least 95%, 96%, 97%, 98%, or 99% identical to SEQ ID NO: 59. In some embodiments, the amino acid sequence of the effector protein comprises or consists of SEQ ID NO: 59.

In certain embodiments, the amino acid sequence of the effector protein is based on SEQ ID NO: 32 and is modified at position 108. In some embodiments, the modification at position 108 is from serine to arginine (S108R). In some embodiments, the amino acid sequence of the effector protein is at least 95%, 96%, 97%, 98%, or 99% identical to SEQ ID NO: 60. In some embodiments, the amino acid sequence of the effector protein comprises or consists of SEQ ID NO: 60.

In certain embodiments, the amino acid sequence of the effector protein is based on SEQ ID NO: 32 and is modified at position 198. In some embodiments, the modification at position 198 is from serine to arginine (S198R). In some embodiments, the amino acid sequence of the effector protein is at least 95%, 96%, 97%, 98%, or 99% identical to SEQ ID NO: 61. In some embodiments, the amino acid sequence of the effector protein comprises or consists of SEQ ID NO: 61.

In certain embodiments, the amino acid sequence of the effector protein is based on SEQ ID NO: 32 and is modified at position 114. In some embodiments, the modification at position 114 is from threonine to arginine (T114R). In some embodiments, the amino acid sequence of the effector protein is at least 95%, 96%, 97%, 98%, or 99% identical to SEQ ID NO: 62. In some embodiments, the amino acid sequence of the effector protein comprises or consists of SEQ ID NO: 62.

In certain embodiments, the amino acid sequence of the effector protein is based on SEQ ID NO: 32 and is modified at position 26 and at position 471. In some embodiments, the modification at position 26 is from leucine to arginine (L26R), and the modification at position 471 is from isoleucine to threonine (I471T). In some embodiments, the amino acid sequence of the effector protein is at least 95%, 96%, 97%, 98%, or 99% identical to SEQ ID NO: 2090. In some embodiments, the amino acid sequence of the effector protein comprises or consists of SEQ ID NO: 2090.

In certain embodiments, the amino acid sequence of the effector protein is based on SEQ ID NO: 32 and is modified at position 471. In some embodiments, the modification at position 471 is from isoleucine to threonine (I471T). In some embodiments, the amino acid sequence of the effector protein is at least 95%, 96%, 97%, 98%, or 99% identical to SEQ ID NO: 2091. In some embodiments, the amino acid sequence of the effector protein comprises or consists of SEQ ID NO: 2091.

In certain embodiments, the amino acid sequence of the effector protein is based on SEQ ID NO: 773 and is modified at position 220. In some embodiments, the modification at position 220 is from aspartic acid to arginine (D220R). In some embodiments, the amino acid sequence of the effector protein is at least 95%, 96%, 97%, 98%, or 99% identical to SEQ ID NO: 775. In some embodiments, the amino acid sequence of the effector protein comprises or consists of SEQ ID NO: 775.

In certain embodiments, the amino acid sequence of the effector protein is based on SEQ ID NO: 773 and is modified at position 58. In some embodiments, the modification at position 58 is from lysine to tryptophane (K58W). In some embodiments, the amino acid sequence of the effector protein is at least 95%, 96%, 97%, 98%, or 99% identical to SEQ ID NO: 776. In some embodiments, the amino acid sequence of the effector protein comprises or consists of SEQ ID NO: 776.

In certain embodiments, the amino acid sequence of the effector protein is based on SEQ ID NO: 773 and is modified at position 218. In some embodiments, the modification at position 218 is from alanine to lysine (A218K). In some embodiments, the amino acid sequence of the effector protein is at least 95%, 96%, 97%, 98%, or 99% identical to SEQ ID NO: 778. In some embodiments, the amino acid sequence of the effector protein comprises or consists of SEQ ID NO: 778.

In certain embodiments, the amino acid sequence of the effector protein is based on SEQ ID NO: 773 and is modified at position 295. In some embodiments, the modification at position 295 is from methionine to tryptophane (M295W). In some embodiments, the amino acid sequence of the effector protein is at least 95%, 96%, 97%, 98%, or 99% identical to SEQ ID NO: 779. In some embodiments, the amino acid sequence of the effector protein comprises or consists of SEQ ID NO: 779.

In certain embodiments, the amino acid sequence of the effector protein is based on SEQ ID NO: 773 and is modified at position 298. In some embodiments, the modification at position 298 is from methionine to leucine (M298L). In some embodiments, the amino acid sequence of the effector protein is at least 95%, 96%, 97%, 98%, or 99% identical to SEQ ID NO: 780. In some embodiments, the amino acid sequence of the effector protein comprises or consists of SEQ ID NO: 780.

In certain embodiments, the amino acid sequence of the effector protein is based on SEQ ID NO: 773 and is modified at position 193. In some embodiments, the modification at position 193 is from asparagine to lysine (N193K). In some embodiments, the amino acid sequence of the effector protein is at least 95%, 96%, 97%, 98%, or 99% identical to SEQ ID NO: 781. In some embodiments, the amino acid sequence of the effector protein comprises or consists of SEQ ID NO: 781.

In certain embodiments, the amino acid sequence of the effector protein is based on SEQ ID NO: 773 and is modified at position 315. In some embodiments, the modification at position 315 is from tyrosine to methionine (Y315M). In some embodiments, the amino acid sequence of the effector protein is at least 95%, 96%, 97%, 98%, or 99% identical to SEQ ID NO: 782. In some embodiments, the amino acid sequence of the effector protein comprises or consists of SEQ ID NO: 782.

In certain embodiments, the amino acid sequence of the effector protein is based on SEQ ID NO: 773 and is modified at position 209. In some embodiments, the modification at position 209 is from serine to phenylalanine (S209F). In some embodiments, the amino acid sequence of the effector protein is at least 95%, 96%, 97%, 98%, or 99% identical to SEQ ID NO: 783. In some embodiments, the amino acid sequence of the effector protein comprises or consists of SEQ ID NO: 783.

In certain embodiments, the amino acid sequence of the effector protein is based on SEQ ID NO: 773 and is modified at position 80. In some embodiments, the modification at position 80 is from isoleucine to lysine (I80K). In some embodiments, the amino acid sequence of the effector protein is at least 95%, 96%, 97%, 98%, or 99% identical to SEQ ID NO: 784. In some embodiments, the amino acid sequence of the effector protein comprises or consists of SEQ ID NO: 784.

In certain embodiments, the amino acid sequence of the effector protein is based on SEQ ID NO: 773 and is modified at position 225. In some embodiments, the modification at position 225 is from glutamine to lysine (E225K). In some embodiments, the amino acid sequence of the effector protein is at least 95%, 96%, 97%, 98%, or 99% identical to SEQ ID NO: 785. In some embodiments, the amino acid sequence of the effector protein comprises or consists of SEQ ID NO: 785.

In certain embodiments, the amino acid sequence of the effector protein is based on SEQ ID NO: 773 and is modified at position 286. In some embodiments, the modification at position 286 is from asparagine to lysine (N286K). In some embodiments, the amino acid sequence of the effector protein is at least 95%, 96%, 97%, 98%, or 99% identical to SEQ ID NO: 786. In some embodiments, the amino acid sequence of the effector protein comprises or consists of SEQ ID NO: 786.

In certain embodiments, the amino acid sequence of the effector protein is based on SEQ ID NO: 773 and is modified at position 306. In some embodiments, the modification at position 306 is from alanine to lysine (A306K). In some embodiments, the amino acid sequence of the effector protein is at least 95%, 96%, 97%, 98%, or 99% identical to SEQ ID NO: 787. In some embodiments, the amino acid sequence of the effector protein comprises or consists of SEQ ID NO: 787.

In certain embodiments, the amino acid sequence of the effector protein is based on SEQ ID NO: 773 and is modified at position 220 and at position 335. In some embodiments, the modification at position 220 is from aspartic acid leucine to arginine (D220R), and the modification at position 335 is from glutamine to glutamic acid (E335Q). In some embodiments, the amino acid sequence of the effector protein is at least 95%, 96%, 97%, 98%, or 99% identical to SEQ ID NO: 793. In some embodiments, the amino acid sequence of the effector protein comprises or consists of SEQ ID NO: 793.

In some embodiments, the effector protein is a Type V Cas protein. In some embodiments, the effector protein is CasM.265466 or a variant thereof. A CasM.265466 is around one third of the size of Cas9. The smaller size of CasM.265466 make it ideal to be packaged together with its corresponding guide RNAs into a single AAV vector, thus overcoming the drawbacks of dual AAV vector systems.

TABLE 16 provides illustrative amino acid sequences of effector proteins. In some embodiments, an effector protein is at least 65%, at least 70%, at least 75%, at least 80%, at least 85%, at least 90%, at least 95%, at least 97%, or at least 98%, at least 99%, or 100% identical to the sequence as set forth in TABLE 16.

In some embodiments, the effector protein is an engineered effector protein and comprises an amino acid sequence that is at least 90%, at least 95%, at least 97%, at least 98%, or at least 99% identical to SEQ ID NO: 773, wherein the polypeptide comprises at least one amino acid substitution relative to SEQ ID NO: 773, wherein the amino acid substitution is at a position selected from K58, 180, T84, K105, N193, C202, S209, G210, A218, D220, E225, C246, N286, M295, M298, A306, Y315, Q360, and a combination thereof. In some embodiments, the polypeptide comprises an amino acid sequence that is 100% identical to SEQ ID NO: 773, with the exception of at least one amino acid substitution relative to SEQ ID NO: 773, wherein the amino acid substitution is a position selected from K58, 180, T84, K105, N193, C202. S209, G210, A218, D220, E225, C246, N286, M295, M298, A306, Y315, Q360, and a combination thereof. In some embodiments, the amino acid substitution is selected from K58X, I80X, T84X, K105X, N193X, C202X, S209X, G210X, A218X, D220X, E225X, C246X, N286X, M295X, M298X, A306X, Y315X, and Q360X, wherein X is selected from R, K, and H.

In some embodiments, the effector protein is an engineered effector protein and comprises an amino acid sequence that is at least 90%, at least 95%, at least 97%, at least 98%, or at least 99% identical to SEQ ID NO: 773, wherein the polypeptide comprises at least one amino acid substitution relative to SEQ ID NO: 773, wherein the amino acid substitution is selected from I80R, T84R, K105R, C202R, G210R, A218R, D220R, E225R, C246R, Q360R, 180K, T84K, G210K, N193K, C202K, A218K, D220K, E225K, C246K, N286K, A306K, Q360K, I80H, T84H, K105H, G210H, C202H, A218H, D220H, E225H, C246H, Q360H, K58W, S209F, M295W, M298L, Y315M, D220R/A306K and D220R/K250N and a combination thereof. In some embodiments, the polypeptide comprises an amino acid sequence that is 100% identical to SEQ ID NO: 773, with the exception of at least one amino acid substitution relative to SEQ ID NO: 773, wherein the amino acid substitution is selected from I80R, T84R, K105R, C202R, G210R, A218R, D220R, E225R, C246R, Q360R, 180K, T84K, G210K, N193K, C202K, A218K, D220K, E225K, C246K, N286K, A306K, Q360K, 1801H, T84H, K105H, G210H, C202H14, A2181H, D2201H, E225H, C246H, Q360H, K58W, S209F, M295W, M298L, Y315M, D220R/A306K, D220R/K250N, D220R/E335Q and a combination thereof. In some embodiments, these engineered effector proteins demonstrate enhanced nuclease activity relative to the wild-type effector protein.

In some embodiments, the effector protein is an engineered effector protein and comprises an amino acid sequence that is at least 90%, at least 95%, at least 97%, at least 98%, or at least 99% identical to SEQ ID NO: 773, wherein the polypeptide comprises at least one amino acid substitution relative to SEQ ID NO: 773, wherein the amino acid substitution is selected from D237A, D418A, D418N, E335A, and E335Q, and a combination thereof. In some embodiments, the polypeptide comprises an amino acid sequence that is 100% identical to SEQ ID NO: 773, with the exception of at least one amino acid substitution relative to SEQ ID NO: 773, wherein the amino acid substitution is selected from D237A, D418A, D418N, E335A, and E335Q, and a combination thereof. In some embodiments, these engineered effector proteins demonstrate reduced or abolished nuclease activity relative to the wild-type effector protein. TABLE 15 provides the exemplary amino acid alterations relative to SEQ ID NO: 773 useful in compositions, systems, and methods described herein.

In some embodiments, the effector protein is an engineered effector protein and comprises an amino acid sequence that is 100% identical to SEQ ID NO: 773, with the exception of at least two amino acid substitutions relative to SEQ ID NO: 773, wherein the amino acid substitutions comprise D220R/E355Q. In some embodiments, the engineered effector protein comprises or consists of SEQ ID NO: 793.

TABLE 16
Exemplary Amino Acid Sequences of
Engineered Variants of CasM.265466

| Effector | | SEQ |
|---|---|---|
| protein | Amino Acid Sequence | ID NO: |
| CasM.265466 | MSVLTRKVQLIPVGDKEERDRVYKYLRDGIEAQNRAMNLYM | 775 |
| D220R | SGLYFAAINEASKEDRKELNQLYSRIATSSKGSAYTTDIEFPTG | |
| | LASTSTLSMAVRQDFTKSLKDGLMYGRVSLPTYRKDNPLFVD | |
| | VRFVALRGTKQKYNGLYHEYKSHTEFLDNLYSSDLKVYIKFA | |
| | NDITFQVIFGNPRKSSALRSEFQNIFEEYYKVCQSSIQFSGTKIIL | |
| | NMAMRIPDKEIELDEDVCVGVDLGIAIPAVCALNKNRYSRVSI | |
| | GSKEDFLRVRTKIRNQRKRLQTNLKSSNGGHGRKKKMKPMD | |
| | RFRDYEANWVQNYNHYVSRQVVDFAVKNKAKYINLENLEGI | |
| | RDDVKNEWLLSNWSYYQLQQYITYKAKTYGIEVRKINPYHTS | |
| | QRCSCCGYEDAGNRPKKEKGQAYFKCLKCGEEMNADFNAAR | |
| | NIAMSTEFQSGKKTKKQKKEQHENK | |
| CasM.265466 | MSVLTRKVQLIPVGDKEERDRVYKYLRDGIEAQNRAMNLYM | 776 |
| K58W | SGLYFAAINEASKEDRWELNQLYSRIATSSKGSAYTTDIEFPTG | |
| | LASTSTLSMAVRQDFTKSLKDGLMYGRVSLPTYRKDNPLFVD | |
| | VRFVALRGTKQKYNGLYHEYKSHTEFLDNLYSSDLKVYIKFA | |
| | NDITFQVIFGNPRKSSALRSEFQNIFEEYYKVCQSSIQFSGTKIIL | |
| | NMAMDIPDKEIELDEDVCVGVDLGIAIPAVCALNKNRYSRVSI | |
| | GSKEDFLRVRTKIRNQRKRLQTNLKSSNGGHGRKKKMKPMD | |
| | RFRDYEANWVQNYNHYVSRQVVDFAVKNKAKYINLENLEGI | |
| | RDDVKNEWLLSNWSYYQLQQYITYKAKTYGIEVRKINPYHTS | |
| | QRCSCCGYEDAGNRPKKEKGQAYFKCLKCGEEMNADFNAAR | |
| | NIAMSTEFQSGKKTKKQKKEQHENK | |
| CasM.265466 | MSVLTRKVQLIPVGDKEERDRVYKYLRDGIEAQNRAMNLYM | 778 |
| A218K | SGLYFAAINEASKEDRKELNQLYSRIATSSKGSAYTTDIEFPTG | |
| | LASTSTLSMAVRQDFTKSLKDGLMYGRVSLPTYRKDNPLFVD | |
| | VRFVALRGTKQKYNGLYHEYKSHTEFLDNLYSSDLKVYIKFA | |
| | NDITFQVIFGNPRKSSALRSEFQNIFEEYYKVCQSSIQFSGTKIIL | |
| | NMKMDIPDKEIELDEDVCVGVDLGIAIPAVCALNKNRYSRVSI | |
| | GSKEDFLRVRTKIRNQRKRLQTNLKSSNGGHGRKKKMKPMD | |
| | RFRDYEANWVQNYNHYVSRQVVDFAVKNKAKYINLENLEGI | |
| | RDDVKNEWLLSNWSYYQLQQYITYKAKTYGIEVRKINPYHTS | |
| | QRCSCCGYEDAGNRPKKEKGQAYFKCLKCGEEMNADFNAAR | |
| | NIAMSTEFQSGKKTKKQKKEQHENK | |
| CasM.265466 | MSVLTRKVQLIPVGDKEERDRVYKYLRDGIEAQNRAMNLYM | 779 |
| M295W | SGLYFAAINEASKEDRKELNQLYSRIATSSKGSAYTTDIEFPTG | |
| | LASTSTLSMAVRQDFTKSLKDGLMYGRVSLPTYRKDNPLFVD | |
| | VRFVALRGTKQKYNGLYHEYKSHTEFLDNLYSSDLKVYIKFA | |
| | NDITFQVIFGNPRKSSALRSEFQNIFEEYYKVCQSSIQFSGTKIIL | |
| | NMAMDIPDKEIELDEDVCVGVDLGIAIPAVCALNKNRYSRVSI | |
| | GSKEDFLRVRTKIRNQRKRLQTNLKSSNGGHGRKKKWKPMD | |
| | RFRDYEANWVQNYNHYVSRQVVDFAVKNKAKYINLENLEGI | |
| | RDDVKNEWLLSNWSYYQLQQYITYKAKTYGIEVRKINPYHTS | |
| | QRCSCCGYEDAGNRPKKEKGQAYFKCLKCGEEMNADFNAAR | |
| | NIAMSTEFQSGKKTKKQKKEQHENK | |
| CasM.265466 | MSVLTRKVQLIPVGDKEERDRVYKYLRDGIEAQNRAMNLYM | 780 |
| M298L | SGLYFAAINEASKEDRKELNQLYSRIATSSKGSAYTTDIEFPTG | |
| | LASTSTLSMAVRQDFTKSLKDGLMYGRVSLPTYRKDNPLFVD | |
| | VRFVALRGTKQKYNGLYHEYKSHTEFLDNLYSSDLKVYIKFA | |
| | NDITFQVIFGNPRKSSALRSEFQNIFEEYYKVCQSSIQFSGTKIIL | |
| | NMAMDIPDKEIELDEDVCVGVDLGIAIPAVCALNKNRYSRVSI | |
| | GSKEDFLRVRTKIRNQRKRLQTNLKSSNGGHGRKKKMKPLDR | |
| | FRDYEANWVQNYNHYVSRQVVDFAVKNKAKYINLENLEGIR | |
| | DDVKNEWLLSNWSYYQLQQYITYKAKTYGIEVRKINPYHTSQ | |
| | RCSCCGYEDAGNRPKKEKGQAYFKCLKCGEEMNADFNAARN | |
| | IAMSTEFQSGKKTKKQKKEQHENK | |
| CasM.265466 | MSVLTRKVQLIPVGDKEERDRVYKYLRDGIEAQNRAMNLYM | 781 |
| N193K | SGLYFAAINEASKEDRKELNQLYSRIATSSKGSAYTTDIEFPTG | |
| | LASTSTLSMAVRQDFTKSLKDGLMYGRVSLPTYRKDNPLFVD | |
| | VRFVALRGTKQKYNGLYHEYKSHTEFLDNLYSSDLKVYIKFA | |
| | NDITFQVIFGNPRKSSALRSEFQKIFEEYYKVCQSSIQFSGTKIIL | |
| | NMAMDIPDKEIELDEDVCVGVDLGIAIPAVCALNKNRYSRVSI | |
| | GSKEDFLRVRTKIRNQRKRLQTNLKSSNGGHGRKKKMKPMD | |
| | RFRDYEANWVQNYNHYVSRQVVDFAVKNKAKYINLENLEGI | |
| | RDDVKNEWLLSNWSYYQLQQYITYKAKTYGIEVRKINPYHTS | |
| | QRCSCCGYEDAGNRPKKEKGQAYFKCLKCGEEMNADFNAAR | |
| | NIAMSTEFQSGKKTKKQKKEQHENK | |
| CasM.265466 | MSVLTRKVQLIPVGDKEERDRVYKYLRDGIEAQNRAMNLYM | 782 |
| Y315M | SGLYFAAINEASKEDRKELNQLYSRIATSSKGSAYTTDIEFPTG | |
| | LASTSTLSMAVRQDFTKSLKDGLMYGRVSLPTYRKDNPLFVD | |
| | VRFVALRGTKQKYNGLYHEYKSHTEFLDNLYSSDLKVYIKFA | |
| | NDITFQVIFGNPRKSSALRSEFQNIFEEYYKVCQSSIQFSGTKIIL | |
| | NMAMDIPDKEIELDEDVCVGVDLGIAIPAVCALNKNRYSRVSI | |
| | GSKEDFLRVRTKIRNQRKRLQTNLKSSNGGHGRKKKMKPMD | |
| | RFRDYEANWVQNYNHMVSRQVVDFAVKNKAKYINLENLEGI | |
| | RDDVKNEWLLSNWSYYQLQQYITYKAKTYGIEVRKINPYHTS | |
| | QRCSCCGYEDAGNRPKKEKGQAYFKCLKCGEEMNADFNAAR | |
| | NIAMSTEFQSGKKTKKQKKEQHENK | |
| CasM.265466 | MSVLTRKVQLIPVGDKEERDRVYKYLRDGIEAQNRAMNLYM | 783 |
| S209F | SGLYFAAINEASKEDRKELNQLYSRIATSSKGSAYTTDIEFPTG | |
| | LASTSTLSMAVRQDFTKSLKDGLMYGRVSLPTYRKDNPLFVD | |
| | VRFVALRGTKQKYNGLYHEYKSHTEFLDNLYSSDLKVYIKFA | |
| | NDITFQVIFGNPRKSSALRSEFQNIFEEYYKVCQSSIQFFGTKIIL | |
| | NMAMDIPDKEIELDEDVCVGVDLGIAIPAVCALNKNRYSRVSI | |
| | GSKEDFLRVRTKIRNQRKRLQTNLKSSNGGHGRKKKMKPMD | |
| | RFRDYEANWVQNYNHYVSRQVVDFAVKNKAKYINLENLEGI | |
| | RDDVKNEWLLSNWSYYQLQQYITYKAKTYGIEVRKINPYHTS | |
| | QRCSCCGYEDAGNRPKKEKGQAYFKCLKCGEEMNADFNAAR | |
| | NIAMSTEFQSGKKTKKQKKEQHENK | |
| CasM.265466 | MSVLTRKVQLIPVGDKEERDRVYKYLRDGIEAQNRAMNLYM | 784 |
| I80K | SGLYFAAINEASKEDRKELNQLYSRIATSSKGSAYTTDKEFPTG | |
| | LASTSTLSMAVRQDFTKSLKDGLMYGRVSLPTYRKDNPLFVD | |
| | VRFVALRGTKQKYNGLYHEYKSHTEFLDNLYSSDLKVYIKFA | |
| | NDITFQVIFGNPRKSSALRSEFQNIFEEYYKVCQSSIQFSGTKIIL | |
| | NMAMDIPDKEIELDEDVCVGVDLGIAIPAVCALNKNRYSRVSI | |
| | GSKEDFLRVRTKIRNQRKRLQTNLKSSNGGHGRKKKMKPMD | |
| | RFRDYEANWVQNYNHYVSRQVVDFAVKNKAKYINLENLEGI | |
| | RDDVKNEWLLSNWSYYQLQQYITYKAKTYGIEVRKINPYHTS | |
| | QRCSCCGYEDAGNRPKKEKGQAYFKCLKCGEEMNADFNAAR | |
| | NIAMSTEFQSGKKTKKQKKEQHENK | |
| CasM.265466 | MSVLTRKVQLIPVGDKEERDRVYKYLRDGIEAQNRAMNLYM | 785 |
| E225K | SGLYFAAINEASKEDRKELNQLYSRIATSSKGSAYTTDIEFPTG | |
| | LASTSTLSMAVRQDFTKSLKDGLMYGRVSLPTYRKDNPLFVD | |
| | VRFVALRGTKQKYNGLYHEYKSHTEFLDNLYSSDLKVYIKFA | |
| | NDITFQVIFGNPRKSSALRSEFQNIFEEYYKVCQSSIQFSGTKIIL | |
| | NMAMDIPDKKIELDEDVCVGVDLGIAIPAVCALNKNRYSRVSI | |
| | GSKEDFLRVRTKIRNQRKRLQTNLKSSNGGHGRKKKMKPMD | |
| | RFRDYEANWVQNYNHYVSRQVVDFAVKNKAKYINLENLEGI | |
| | RDDVKNEWLLSNWSYYQLQQYITYKAKTYGIEVRKINPYHTS | |
| | QRCSCCGYEDAGNRPKKEKGQAYFKCLKCGEEMNADFNAAR | |
| | NIAMSTEFQSGKKTKKQKKEQHENK | |
| CasM.265466 | MSVLTRKVQLIPVGDKEERDRVYKYLRDGIEAQNRAMNLYM | 786 |
| N286K | SGLYFAAINEASKEDRKELNQLYSRIATSSKGSAYTTDIEFPTG | |
| | LASTSTLSMAVRQDFTKSLKDGLMYGRVSLPTYRKDNPLFVD | |
| | VRFVALRGTKQKYNGLYHEYKSHTEFLDNLYSSDLKVYIKFA | |
| | NDITFQVIFGNPRKSSALRSEFQNIFEEYYKVCQSSIQFSGTKIIL | |
| | NMAMDIPDKEIELDEDVCVGVDLGIAIPAVCALNKNRYSRVSI | |
| | GSKEDFLRVRTKIRNQRKRLQTNLKSSKGGHGRKKKMKPMD | |
| | RFRDYEANWVQNYNHYVSRQVVDFAVKNKAKYINLENLEGI | |
| | RDDVKNEWLLSNWSYYQLQQYITYKAKTYGIEVRKINPYHTS | |
| | QRCSCCGYEDAGNRPKKEKGQAYFKCLKCGEEMNADFNAAR | |
| | NIAMSTEFQSGKKTKKQKKEQHENK | |
| CasM.265466 | MSVLTRKVQLIPVGDKEERDRVYKYLRDGIEAQNRAMNLYM | 787 |
| A306K | SGLYFAAINEASKEDRKELNQLYSRIATSSKGSAYTTDIEFPTG | |
| | LASTSTLSMAVRQDFTKSLKDGLMYGRVSLPTYRKDNPLFVD | |
| | VRFVALRGTKQKYNGLYHEYKSHTEFLDNLYSSDLKVYIKFA | |
| | NDITFQVIFGNPRKSSALRSEFQNIFEEYYKVCQSSIQFSGTKIIL | |
| | NMAMDIPDKEIELDEDVCVGVDLGIAIPAVCALNKNRYSRVSI | |
| | GSKEDFLRVRTKIRNQRKRLQTNLKSSNGGHGRKKKMKPMD | |
| | RFRDYEKNWVQNYNHYVSRQVVDFAVKNKAKYINLENLEGI | |
| | RDDVKNEWLLSNWSYYQLQQYITYKAKTYGIEVRKINPYHTS | |
| | QRCSCCGYEDAGNRPKKEKGQAYFKCLKCGEEMNADFNAAR | |
| | NIAMSTEFQSGKKTKKQKKEQHENK | |
| CasM.265466 | MSVLTRKVQLIPVGDKEERDRVYKYLRDGIEAQNRAMNLYM | 788 |
| E335Q | SGLYFAAINEASKEDRKELNQLYSRIATSSKGSAYTTDIEFPTG | |
| | LASTSTLSMAVRQDFTKSLKDGLMYGRVSLPTYRKDNPLFVD | |
| | VRFVALRGTKQKYNGLYHEYKSHTEFLDNLYSSDLKVYIKFA | |
| | NDITFQVIFGNPRKSSALRSEFQNIFEEYYKVCQSSIQFSGTKIIL | |
| | NMAMDIPDKEIELDEDVCVGVDLGIAIPAVCALNKNRYSRVSI | |
| | GSKEDFLRVRTKIRNQRKRLQTNLKSSNGGHGRKKKMKPMD | |
| | RFRDYEANWVQNYNHYVSRQVVDFAVKNKAKYINLQNLEGI | |
| | RDDVKNEWLLSNWSYYQLQQYITYKAKTYGIEVRKINPYHTS | |
| | QRCSCCGYEDAGNRPKKEKGQAYFKCLKCGEEMNADFNAAR | |
| | NIAMSTEFQSGKKTKKQKKEQHENK | |
| CasM.265466 | MSVLTRKVQLIPVGDKEERDRVYKYLRDGIEAQNRAMNLYM | 789 |
| D237A | SGLYFAAINEASKEDRKELNQLYSRIATSSKGSAYTTDIEFPTG | |
| | LASTSTLSMAVRQDFTKSLKDGLMYGRVSLPTYRKDNPLFVD | |
| | VRFVALRGTKQKYNGLYHEYKSHTEFLDNLYSSDLKVYIKFA | |
| | NDITFQVIFGNPRKSSALRSEFQNIFEEYYKVCQSSIQFSGTKIIL | |
| | NMAMDIPDKEIELDEDVCVGVALGIAIPAVCALNKNRYSRVSI | |
| | GSKEDFLRVRTKIRNQRKRLQTNLKSSNGGHGRKKKMKPMD | |
| | RFRDYEANWVQNYNHYVSRQVVDFAVKNKAKYINLENLEGI | |
| | RDDVKNEWLLSNWSYYQLQQYITYKAKTYGIEVRKINPYHTS | |
| | QRCSCCGYEDAGNRPKKEKGQAYFKCLKCGEEMNADFNAAR | |
| | NIAMSTEFQSGKKTKKQKKEQHENK | |
| CasM.265466 | MSVLTRKVQLIPVGDKEERDRVYKYLRDGIEAQNRAMNLYM | 790 |
| D418A | SGLYFAAINEASKEDRKELNQLYSRIATSSKGSAYTTDIEFPTG | |
| | LASTSTLSMAVRQDFTKSLKDGLMYGRVSLPTYRKDNPLFVD | |
| | VRFVALRGTKQKYNGLYHEYKSHTEFLDNLYSSDLKVYIKFA | |
| | NDITFQVIFGNPRKSSALRSEFQNIFEEYYKVCQSSIQFSGTKIIL | |
| | NMAMDIPDKEIELDEDVCVGVDLGIAIPAVCALNKNRYSRVSI | |
| | GSKEDFLRVRTKIRNQRKRLQTNLKSSNGGHGRKKKMKPMD | |
| | RFRDYEANWVQNYNHYVSRQVVDFAVKNKAKYINLENLEGI | |
| | RDDVKNEWLLSNWSYYQLQQYITYKAKTYGIEVRKINPYHTS | |
| | QRCSCCGYEDAGNRPKKEKGQAYFKCLKCGEEMNAAFNAAR | |
| | NIAMSTEFQSGKKTKKQKKEQHENK | |
| CasM.265466 | MSVLTRKVQLIPVGDKEERDRVYKYLRDGIEAQNRAMNLYM | 791 |
| D418N | SGLYFAAINEASKEDRKELNQLYSRIATSSKGSAYTTDIEFPTG | |
| | LASTSTLSMAVRQDFTKSLKDGLMYGRVSLPTYRKDNPLFVD | |
| | VRFVALRGTKQKYNGLYHEYKSHTEFLDNLYSSDLKVYIKFA | |
| | NDITFQVIFGNPRKSSALRSEFQNIFEEYYKVCQSSIQFSGTKIIL | |
| | NMAMDIPDKEIELDEDVCVGVDLGIAIPAVCALNKNRYSRVSI | |
| | GSKEDFLRVRTKIRNQRKRLQTNLKSSNGGHGRKKKMKPMD | |
| | RFRDYEANWVQNYNHYVSRQVVDFAVKNKAKYINLENLEGI | |
| | RDDVKNEWLLSNWSYYQLQQYITYKAKTYGIEVRKINPYHTS | |
| | QRCSCCGYEDAGNRPKKEKGQAYFKCLKCGEEMNANFNAAR | |
| | NIAMSTEFQSGKKTKKQKKEQHENK | |
| CasM.265466 | MSVLTRKVQLIPVGDKEERDRVYKYLRDGIEAQNRAMNLYM | 792 |
| E335A | SGLYFAAINEASKEDRKELNQLYSRIATSSKGSAYTTDIEFPTG | |
| | LASTSTLSMAVRQDFTKSLKDGLMYGRVSLPTYRKDNPLFVD | |
| | VRFVALRGTKQKYNGLYHEYKSHTEFLDNLYSSDLKVYIKFA | |
| | NDITFQVIFGNPRKSSALRSEFQNIFEEYYKVCQSSIQFSGTKIIL | |
| | NMAMDIPDKEIELDEDVCVGVDLGIAIPAVCALNKNRYSRVSI | |
| | GSKEDFLRVRTKIRNQRKRLQTNLKSSNGGHGRKKKMKPMD | |
| | RFRDYEANWVQNYNHYVSRQVVDFAVKNKAKYINLANLEGI | |
| | RDDVKNEWLLSNWSYYQLQQYITYKAKTYGIEVRKINPYHTS | |
| | QRCSCCGYEDAGNRPKKEKGQAYFKCLKCGEEMNADFNAAR | |
| | NIAMSTEFQSGKKTKKQKKEQHENK | |
| CasM.265466 | MSVLTRKVQLIPVGDKEERDRVYKYLRDGIEAQNRAMNLYM | 793 |
| D220R- | SGLYFAAINEASKEDRKELNQLYSRIATSSKGSAYTTDIEFPTG | |
| E335Q | LASTSTLSMAVRQDFTKSLKDGLMYGRVSLPTYRKDNPLFVD | |
| | VRFVALRGTKQKYNGLYHEYKSHTEFLDNLYSSDLKVYIKFA | |
| | NDITFQVIFGNPRKSSALRSEFQNIFEEYYKVCQSSIQFSGTKIIL | |
| | NMAMRIPDKEIELDEDVCVGVDLGIAIPAVCALNKNRYSRVSI | |
| | GSKEDFLRVRTKIRNQRKRLQTNLKSSNGGHGRKKKMKPMD | |
| | RFRDYEANWVQNYNHYVSRQVVDFAVKNKAKYINLQNLEGI | |
| | RDDVKNEWLLSNWSYYQLQQYITYKAKTYGIEVRKINPYHTS | |
| | QRCSCCGYEDAGNRPKKEKGQAYFKCLKCGEEMNADFNAAR | |
| | NIAMSTEFQSGKKTKKQKKEQHENK | |

TABLE 17
Exemplary Amino Acid Alterations Relative to SEQ ID NO: 773

| Effects | Amino Acid Alterations |
|---|---|
| | At least one substitution (i.e., with R, K or H) selected |
| | from K58, I80, T84, K105, N193, C202, S209, G210, |
| | A218, D220, E225, C246, N286, M295, M298, A306, |
| | Y315, and Q360 |
| Enhanced nuclease activity | I80R, T84R, K105R, C202R, G210R, A218R, D220R, |
| relative to the wild-type effector | E225R, C246R, Q360R, I80K, T84K, G210K, N193K, |
| protein | C202K, A218K, D220K, E225K, C246K, N286K, |
| | A306K, Q360K, I80H, T84H, K105H, G210H, C202H, |
| | A218H, D220H, E225H, C246H, Q360H, K58W, |
| | S209F, M295W, M298L, Y315M |
| | Double mutations: D220R/A306K, D220R/K250N |
| Reduced or abolished nuclease | D237A, D418A, D418N, E335A, E335Q |
| activity relative to the wild-type | |
| effector protein | |

TABLE 18 provides illustrative amino acid sequences of effector proteins. In some embodiments, an effector protein is at least 65%, at least 70%, at least 75%, at least 80%, at least 85%, at least 90%, at least 95%, at least 97%, or at least 98%, at least 99%, or 100% identical to the sequence as set forth in TABLE 18.

In some embodiments, the effector protein is an engineered effector protein and comprises an amino acid sequence that is at least 90%, at least 95%, at least 97%, at least 98%, or at least 99% identical to SEQ ID NO: 32, wherein the polypeptide comprises at least one amino acid substitution relative to SEQ ID NO: 32, wherein the amino acid substitution is at a position selected from 12, T5, K15, R18, 1120, S21, L26, N30, E33, E34, A35, K37, K38, R41, N43, Q54, Q79R, K92E, K99R, S108, E109, H110, G111, D113, T114, P116, K118, E119, A121, N132, K135, Q138, V139, N148, L149, E157, E164, E166, E170, Y180, L182, Q183, K184, S186, K189, S196, S198, K200, 1203, S205, K206, Y207, H208, N209, Y220, S223, E258, K281, K348, N355, S362, I406, I435, I471, I489, Y490, F491, D495, K496, K498, K500, D501, V502, K504, S505, D506, V521, N568, S579, Q612, S638, F701, P707, and a combination thereof. In some embodiments, the polypeptide comprises an amino acid sequence that is 100% identical to SEQ ID NO: 32, with the exception of at least one amino acid substitution relative to SEQ ID NO: 32, wherein the amino acid substitution is at a position selected from 12, T5, K15, R18, H20, S21, L26, N30, E33, E34, A35, K37, K38, R41, N43, Q54, Q79R, K92E, K99R, S108, E109, H110, G111, D113, T114, P116, K118, E119, A121, N132, K135, Q138, V139. N148, L149, E157, E164, E166, E170, Y180, L182, Q183, K184, S186, K189, S196, S198, K200, 1203, S205, K206, Y207, H208, N209, Y220, S223, E258, K281, K348, N355, S362, N406, K435, 1471, 1489, Y490, F491, D495, K496, K498, K500, D501, V502, K504, S505, D506, V521, N568, S579, Q612, S638, F701, P707, and a combination thereof. In some embodiments, the amino acid substitution is selected from I2X, T5X, K15X, R18X, 120X, S21X, L26X, N30X, E33X, E34X, A35X, K37X, K38X, R41X, N43X, Q54X, Q79RX, K92EX, K99RX, S108X, E109X, H110X, G111X, D113X, T114X, P116X, K118X, E119X, A121X. N132X, K135X. Q138X, V139X, N148X, L149X, E157X, E164X, E166X, E170X, Y180X, L182X, Q183X, K184X, S186K, K189X, S196X, S198X, K200X, 1203X, S205X, K206X, Y207X, 1208X, N209X, Y220X, S223X, E258X, K281X, K348X, N355X, S362X, N406X, K435X, I471X, 1489X, Y490X, F491X, D495X, K496X, K498X, K500X, D501X, V502K, K504X, S505X, D506X, V521X, N568X, S579X, Q612X, S638X, F701X, P707X, wherein X is selected from R, K. and H.

In some embodiments, the effector protein is an engineered effector protein and comprises an amino acid sequence that is at least 90%, at least 95%, at least 97%, at least 98%, or at least 99% identical to SEQ ID NO: 32, wherein the polypeptide comprises at least one amino acid substitution relative to SEQ ID NO: 32 wherein the amino acid substitution is selected from T5R, L26R, L26K, A121Q, V139R, S198R, S223P, E258K, 1471T, S579R, F701R, P707R, K189P, S638K, Q54R, Q79R, Y220S, N406K, E119S, K92E. K435Q, N568D, and V521T, and a combination thereof. In some embodiments, the polypeptide comprises an amino acid sequence that is 100% identical to SEQ ID NO: 32, with the exception of at least one amino acid substitution relative to SEQ ID NO: 32, wherein the amino acid substitution is selected from T5R, L26R, L26K, A121Q, V139R, S198R, S223P, E258K, 1471T. S579R, F701R, P707R, K189P, S638K, Q54R, Q79R, Y220S, N406K, E119S, K92E, K435Q, N568D, and V521T, and a combination thereof. In some embodiments, these engineered effector proteins demonstrate enhanced nuclease activity relative to the wild-type effector protein.

In some embodiments, the effector protein is an engineered effector protein and comprises an amino acid sequence that is at least 90%, at least 95%, at least 97%, at least 98%, or at least 99% identical to SEQ ID NO: 32, wherein the polypeptide comprises at least one amino acid substitution relative to SEQ ID NO: 32 wherein the amino acid substitution is selected from L26K/A121Q, L26R/A121Q, K99R/L149R, K99R/N148R, L149R/H208R, S362R/L26R L26R/N148R, L26R/H208R, N30R/N148R, L26R/K99R, L26R/P707R, L26R/1L149R, L26R/N30R, L26R/N355R, L26R/K281R, L26R/S108R, L26R/K348R, T5R/V139R, 12R/V139R, K99R/S186R, L26R/A673G, L26R/Q674R, S579R/L26K, F701R/E258K, T5R/L26K, L26R/K435Q, L26K/E567Q, L26R/G685R, L26R/Q674K, L26R/P699R, L26R/T70E, L26R/Q232R, L26R/T252R, L26R/P679R, L26R/E83K, L26R/E73P, L26R/K248E, L26R, T5R/S223P, S579R/S223P, L26R/S223P, T5R/A121Q, L26R/A696R, S198R/1471T, L26R/N153R, L26R/E682R, L26R/D703R, Q612R/126K, L26R/1471T, K348R/L26K, S579R/T471T, L26R/V228R, T5R/S638K, S579R/K189P, S579R1E258K, L26R/K260R, 126R/S638K, S579R/Y220S, T5R/I471 T, 126R/F233R, L26R/V521T, F701R/A121Q, L26R/G361R, S198R/E258K, L26R/S472R, T5R/Y220S, L26R/A150K, L26R/S684R, L26R/E157R, L26R/K248R, F701R/L26K, S198R/N406K, S198R/Y220S, S198R/S638K, S198R/V521 T, S579R/A121Q, K348R/Y220S, S198R1K189P, 126R/E242R, L26R/K678R, T5R/1406K, L26R/I158K, T5R/V521T, L26R/N259R, L26R/K257R, L26R/K256R, T5R/K189P, L26R/C405R, S579R/V521T, S579R/N406K, T5R/K92E, T5R/E258K, L26R/197R, S579R/S638K, T5R/K435Q, F701R/S638K, L26R/L236R, L26R, I1471T, F701R/I471T, Q612R/S223P, F701R/S223P, S198R/E119S, S579R/K92E, L26R/E715R, Q612R/471T, F701R/Y220S, S198R/S223P, and L26R/K266R, and a combination thereof. In some embodiments, the polypeptide comprises an amino acid sequence that is 100% identical to SEQ ID NO: 32, with the exception of at least one amino acid substitution relative to SEQ ID NO: 32, wherein the amino acid substitution is selected from L26K/A121Q, L26R/A121Q, K99R/L149R, K99R/N148R, L149R/H208R, S362R/L26R L26R/N148R, L26R/H208R, N30R/N148R, L26R/K99R, L26R/P707R, L26R/L149R, L26R/N30R, L26R/N355R, L26R/K281R, L26R/S108R, L26R/K348R, T5R/V139R, I2R/V139R, K99R/S186R, L26R/A673G, L26K/E567Q, L26R/Q674R, S579R/L26K, F701R/E258K, T5R/L26K, L26R/K435Q, L26R/G685R, L26R/Q674K, L26R/P699R, L26R/T70E, L26R/Q232R, L26R/T252R, L26R/P679R, L26R/E83K, L26R/E73P, L26R/K248E, L26R, T5R′ S223P, S579R/S223P, L26R/S223P, T5R/A121Q L26R/A696R, S198R/1471T, L26R/N153R, L26R/E682R, L26R/D703R, Q612R/L26K, L26R/1471T, K348R/126K, S579R/I471T, L26R/V228R, T5R/S638K, S579R/K189P, S579R/E258K, L26R/K260R, L26R/S638K, S579R/Y220S, T5R/I471T, L26R/F233R, L26R/V521T, F701R/A121Q, L26R/G361R, S198R/E258K, L26R/S472R, T5R/Y220S, L26R/A150K, L26R/S684R, L26R/E157R, L26R/K248R, F701R/L26K, S198R/N406K, S198R/Y220S, S198R/S638K, S198R/V521T, S579R/A121Q, K348R/Y220S, S198R/K189P, L26R/E242R, L26R/K678R, T5R/N406K, L26R/T158K, T5R/V521T, L26R/N259R, L26R/K257R, L26R/K256R, T5R/K189P, L26R/C405R, S579R/V521 T, S579R/N406K, T5R/K92E, T5R/E258K, L26R/I97R, S579R/S638K, T5R/K435Q, F701R/S638K, L26R/L236R, L26R/1471T, F701R/I471T, Q612R/S223P, F701R/S223P, S198R/E119S, S579R/K92E, L26R/E715R, Q612R/1471T, F701R/Y220S, S198R/S223P, and L26R/K266R, and a combination thereof. In some embodiments, these engineered effector proteins demonstrate enhanced nuclease activity relative to the wild-type effector protein.

In some embodiments, the polypeptide comprises an amino acid sequence that is 100% identical to SEQ ID NO: 32, with the exception of at least two amino acid substitutions relative to SEQ ID NO: 32, wherein the amino acid substitutions comprise L26K/E567Q. In some embodiments, the polypeptide comprises or consists of SEQ ID NO: 794.

In some embodiments, the effector protein is an engineered effector protein and comprises an amino acid sequence that is at least 90%, at least 95%, at least 97%, at least 98%, or at least 99% identical to SEQ ID NO: 32, wherein the polypeptide comprises at least one amino acid substitution relative to SEQ ID NO: 32 wherein the amino acid substitution is selected from E157A, E164A, E164L, E166A, E166I, E170A, 1489A, 1489S, Y490S, Y490A, F491A, F491S, F491G, D495G, D495R, D495K, K496A, K496S, K498A, K498S, K500A, K500S, D501R, D501 G, D501K, V502A, V502S, K504A, K504S, S505R, D506A, and a combination thereof. In some embodiments, the polypeptide comprises an amino acid sequence that is 100% identical to SEQ ID NO: 32, with the exception of at least one amino acid substitution relative to SEQ ID NO: 32, wherein the amino acid substitution is selected from E157A, E164A, E164L, E166A, E166I, E170A, 1489A, 1489S, Y490S, Y490A, F491A, F491S, F491G, D495G, D495R, D495K, K496A, K496S, K498A, K498S, K500A, K500S, D501R, D501G, D501K, V502A, V502S, K504A, K504S, S505R, D506A, and a combination thereof. In some embodiments, these engineered effector proteins comprise a nickase activity.

In some embodiments, the effector protein is an engineered effector protein and comprises an amino acid sequence that is at least 90%, at least 95%, at least 97%, at least 98%, or at least 99% identical to SEQ ID NO: 32, wherein amino acids S478-S505 have been deleted. In some embodiments, the effector protein is an engineered effector protein that is at least 90%, at least 95%, at least 97%, at least 98%, or at least 99% identical to SEQ ID NO: 32, wherein amino acids S478-S505 have been deleted and replaced with SDLYIERGGDPRDVHQQVETKPKGKRKSEIRILKIR (SEQ ID NO: 205) or SDYIVDHGGDPEKVFFETKSKKDKTKRYKRR (SEQ ID NO: 206) In some embodiments, the effector protein is an engineered effector protein and comprises an amino acid sequence that is at least 90%, at least 95%, at least 97%, at least 98%, at least 99% identical, or is 100% identical to SEQ ID NO: 203. In some embodiments, the effector protein is an engineered effector protein and comprises an amino acid sequence that is at least 90%, at least 95%, at least 97%, at least 98%, at least 99% identical, or is 100% identical to SEQ ID NO: 204.

In some embodiments, the effector protein is an engineered effector protein and comprises an amino acid sequence that is at least 90%, at least 95%, at least 97%, at least 98%, or at least 99% identical to SEQ ID NO: 32, wherein the polypeptide comprises at least one amino acid substitution relative to SEQ ID NO: 32 wherein the amino acid substitution is selected from D369A, D369N, D658A, D658N, E567A, E567Q, and a combination thereof. In some embodiments, the polypeptide comprises an amino acid sequence that is 100% identical to SEQ ID NO: 32, with the exception of at least one amino acid substitution relative to SEQ ID NO: 32, wherein the amino acid substitution is selected from D369A, D369N, D658A, D658N, E567A, E567Q, and a combination thereof. In some embodiments, these engineered effector proteins demonstrate reduced or abolished nuclease activity relative to the wild-type effector protein. TABLE 18 provides the exemplary amino acid alterations relative to SEQ ID NO: 32 useful in compositions, systems, and methods described herein.

TABLE 18
Exemplary Amino Acid Sequences of
Engineered Variants of CasPhi.12

| Effector | | SEQ |
|---|---|---|
| protein | Amino Acid Sequence | ID NO: |
| CasPhi.12 | MIKPTVSQFLTPGFKLIRNHSRTAGRKLKNEGEEACKKFVREN | 34 |
| L26R | EIPKDECPNFQGGPAIANIIAKSREFTEWEIYQSSLAIQEVIFTLP | |
| | KDKLPEPILKEEWRAQWLSEHGLDTVPYKEAAGLNLIIKNAVN | |
| | TYKGVQVKVDNKNKNNLAKINRKNEIAKLNGEQEISFEEIKAF | |
| | DDKGYLLQKPSPNKSIYCYQSVSPKPFITSKYHNVNLPEEYIGY | |
| | YRKSNEPIVSPYQFDRLRIPIGEPGYVPKWQYTFLSKKENKRRK | |
| | LSKRIKNVSPILGIICIKKDWCVFDMRGLLRTNHWKKYHKPTD | |
| | SINDLFDYFTGDPVIDTKANVVRFRYKMENGIVNYKPVREKKG | |
| | KELLENICDQNGSCKLATVDVGQNNPVAIGLFELKKVNGELTK | |
| | TLISRHPTPIDFCNKITAYRERYDKLESSIKLDAIKQLTSEQKIEV | |
| | DNYNNNFTPQNTKQIVCSKLNINPNDLPWDKMISGTHFISEKA | |
| | QVSNKSEIYFTSTDKGKTKDVMKSDYKWFQDYKPKLSKEVRD | |
| | ALSDIEWRLRRESLEFNKLSKSREQDARQLANWISSMCDVIGIE | |
| | NLVKKNNFFGGSGKREPGWDNFYKPKKENRWWINAIHKALT | |
| | ELSQNKGKRVILLPAMRTSITCPKCKYCDSKNRNGEKFNCLKC | |
| | GIELNADIDVATENLATVAITAQSMPKPTCERSGDAKKPVRAR | |
| | KAKAPEFHDKLAPSYTVVLREAV | |
| 3x Flag- | MDYKDHDGDYKDHDIDYKDDDDKMAPKKKRKVGIHGVPAA | 35 |
| SV40NLS- | MIKPTVSQFLTPGFKLIRNHSRTAGRKLKNEGEEACKKFVREN | |
| CasPhi12 | EIPKDECPNFQGGPAIANIIAKSREFTEWEIYQSSLAIQEVIFTLP | |
| L26R- | KDKLPEPILKEEWRAQWLSEHGLDTVPYKEAAGLNLIIKNAVN | |
| NLS | TYKGVQVKVDNKNKNNLAKINRKNEIAKLNGEQEISFEEIKAF | |
| | DDKGYLLQKPSPNKSIYCYQSVSPKPFITSKYHNVNLPEEYIGY | |
| | YRKSNEPIVSPYQFDRLRIPIGEPGYVPKWQYTFLSKKENKRRK | |
| | LSKRIKNVSPILGIICIKKDWCVFDMRGLLRTNHWKKYHKPTD | |
| | SINDLFDYFTGDPVIDTKANVVRFRYKMENGIVNYKPVREKKG | |
| | KELLENICDQNGSCKLATVDVGQNNPVAIGLFELKKVNGELTK | |
| | TLISRHPTPIDFCNKITAYRERYDKLESSIKLDAIKQLTSEQKIEV | |
| | DNYNNNFTPQNTKQIVCSKLNINPNDLPWDKMISGTHFISEKA | |
| | QVSNKSEIYFTSTDKGKTKDVMKSDYKWFQDYKPKLSKEVRD | |
| | ALSDIEWRLRRESLEFNKLSKSREQDARQLANWISSMCDVIGIE | |
| | NLVKKNNFFGGSGKREPGWDNFYKPKKENRWWINAIHKALT | |
| | ELSQNKGKRVILLPAMRTSITCPKCKYCDSKNRNGEKFNCLKC | |
| | GIELNADIDVATENLATVAITAQSMPKPTCERSGDAKKPVRAR | |
| | KAKAPEFHDKLAPSYTVVLREAVKGRRPRKRPARQKRKRNS | |
| CasPhi.12 | MIKPTVSQFLTPGFKLIRNHSRTAGLKLKNEGEEACKKFVREN | 45 |
| E567A | EIPKDECPNFQGGPAIANIIAKSREFTEWEIYQSSLAIQEVIFTLP | |
| | KDKLPEPILKEEWRAQWLSEHGLDTVPYKEAAGLNLIIKNAVN | |
| | TYKGVQVKVDNKNKNNLAKINRKNEIAKLNGEQEISFEEIKAF | |
| | DDKGYLLQKPSPNKSIYCYQSVSPKPFITSKYHNVNLPEEYIGY | |
| | YRKSNEPIVSPYQFDRLRIPIGEPGYVPKWQYTFLSKKENKRRK | |
| | LSKRIKNVSPILGIICIKKDWCVFDMRGLLRTNHWKKYHKPTD | |
| | SINDLFDYFTGDPVIDTKANVVRFRYKMENGIVNYKPVREKKG | |
| | KELLENICDQNGSCKLATVDVGQNNPVAIGLFELKKVNGELTK | |
| | TLISRHPTPIDFCNKITAYRERYDKLESSIKLDAIKQLTSEQKIEV | |
| | DNYNNNFTPQNTKQIVCSKLNINPNDLPWDKMISGTHFISEKA | |
| | QVSNKSEIYFTSTDKGKTKDVMKSDYKWFQDYKPKLSKEVRD | |
| | ALSDIEWRLRRESLEFNKLSKSREQDARQLANWISSMCDVIGIA | |
| | NLVKKNNFFGGSGKREPGWDNFYKPKKENRWWINAIHKALT | |
| | ELSQNKGKRVILLPAMRTSITCPKCKYCDSKNRNGEKFNCLKC | |
| | GIELNADIDVATENLATVAITAQSMPKPTCERSGDAKKPVRAR | |
| | KAKAPEFHDKLAPSYTVVLREAV | |
| CasPhi.12 | MIKPTVSQFLTPGFKLIRNHSRTAGLKLKNEGEEACKKFVREN | 46 |
| E567Q | EIPKDECPNFQGGPAIANIIAKSREFTEWEIYQSSLAIQEVIFTLP | |
| | KDKLPEPILKEEWRAQWLSEHGLDTVPYKEAAGLNLIIKNAVN | |
| | TYKGVQVKVDNKNKNNLAKINRKNEIAKLNGEQEISFEEIKAF | |
| | DDKGYLLQKPSPNKSIYCYQSVSPKPFITSKYHNVNLPEEYIGY | |
| | YRKSNEPIVSPYQFDRLRIPIGEPGYVPKWQYTFLSKKENKRRK | |
| | LSKRIKNVSPILGIICIKKDWCVFDMRGLLRTNHWKKYHKPTD | |
| | SINDLFDYFTGDPVIDTKANVVRFRYKMENGIVNYKPVREKKG | |
| | KELLENICDQNGSCKLATVDVGQNNPVAIGLFELKKVNGELTK | |
| | TLISRHPTPIDFCNKITAYRERYDKLESSIKLDAIKQLTSEQKIEV | |
| | DNYNNNFTPQNTKQIVCSKLNINPNDLPWDKMISGTHFISEKA | |
| | QVSNKSEIYFTSTDKGKTKDVMKSDYKWFQDYKPKLSKEVRD | |
| | ALSDIEWRLRRESLEFNKLSKSREQDARQLANWISSMCDVIGI | |
| | QNLVKKNNFFGGSGKREPGWDNFYKPKKENRWWINAIHKAL | |
| | TELSQNKGKRVILLPAMRTSITCPKCKYCDSKNRNGEKFNCLK | |
| | CGIELNADIDVATENLATVAITAQSMPKPTCERSGDAKKPVRA | |
| | RKAKAPEFHDKLAPSYTVVLREAV | |
| CasPhi.12 | MIKPTVSQFLTPGFKLIRNHSRTAGLKLKNEGEEACKKFVREN | 54 |
| E109R | EIPKDECPNFQGGPAIANIIAKSREFTEWEIYQSSLAIQEVIFTLP | |
| | KDKLPEPILKEEWRAQWLSRHGLDTVPYKEAAGLNLIIKNAV | |
| | NTYKGVQVKVDNKNKNNLAKINRKNEIAKLNGEQEISFEEIKA | |
| | FDDKGYLLQKPSPNKSIYCYQSVSPKPFITSKYHNVNLPEEYIG | |
| | YYRKSNEPIVSPYQFDRLRIPIGEPGYVPKWQYTFLSKKENKRR | |
| | KLSKRIKNVSPILGIICIKKDWCVFDMRGLLRTNHWKKYHKPT | |
| | DSINDLFDYFTGDPVIDTKANVVRFRYKMENGIVNYKPVREKK | |
| | GKELLENICDQNGSCKLATVDVGQNNPVAIGLFELKKVNGELT | |
| | KTLISRHPTPIDFCNKITAYRERYDKLESSIKLDAIKQLTSEQKIE | |
| | VDNYNNNFTPQNTKQIVCSKLNINPNDLPWDKMISGTHFISEK | |
| | AQVSNKSEIYFTSTDKGKTKDVMKSDYKWFQDYKPKLSKEVR | |
| | DALSDIEWRLRRESLEFNKLSKSREQDARQLANWISSMCDVIGI | |
| | ENLVKKNNFFGGSGKREPGWDNFYKPKKENRWWINAIHKAL | |
| | TELSQNKGKRVILLPAMRTSITCPKCKYCDSKNRNGEKFNCLK | |
| | CGIELNADIDVATENLATVAITAQSMPKPTCERSGDAKKPVRA | |
| | RKAKAPEFHDKLAPSYTVVLREAV | |
| CasPhi.12 | MIKPTVSQFLTPGFKLIRNHSRTAGLKLKNEGEEACKKFVREN | 55 |
| H208R | EIPKDECPNFQGGPAIANIIAKSREFTEWEIYQSSLAIQEVIFTLP | |
| | KDKLPEPILKEEWRAQWLSEHGLDTVPYKEAAGLNLIIKNAVN | |
| | TYKGVQVKVDNKNKNNLAKINRKNEIAKLNGEQEISFEEIKAF | |
| | DDKGYLLQKPSPNKSIYCYQSVSPKPFITSKYRNVNLPEEYIGY | |
| | YRKSNEPIVSPYQFDRLRIPIGEPGYVPKWQYTFLSKKENKRRK | |
| | LSKRIKNVSPILGIICIKKDWCVFDMRGLLRTNHWKKYHKPTD | |
| | SINDLFDYFTGDPVIDTKANVVRFRYKMENGIVNYKPVREKKG | |
| | KELLENICDQNGSCKLATVDVGQNNPVAIGLFELKKVNGELTK | |
| | TLISRHPTPIDFCNKITAYRERYDKLESSIKLDAIKQLTSEQKIEV | |
| | DNYNNNFTPQNTKQIVCSKLNINPNDLPWDKMISGTHFISEKA | |
| | QVSNKSEIYFTSTDKGKTKDVMKSDYKWFQDYKPKLSKEVRD | |
| | ALSDIEWRLRRESLEFNKLSKSREQDARQLANWISSMCDVIGIE | |
| | NLVKKNNFFGGSGKREPGWDNFYKPKKENRWWINAIHKALT | |
| | ELSQNKGKRVILLPAMRTSITCPKCKYCDSKNRNGEKFNCLKC | |
| | GIELNADIDVATENLATVAITAQSMPKPTCERSGDAKKPVRAR | |
| | KAKAPEFHDKLAPSYTVVLREAV | |
| CasPhi.12 | MIKPTVSQFLTPGFKLIRNHSRTAGLKLKNEGEEACKKFVREN | 56 |
| K184R | EIPKDECPNFQGGPAIANIIAKSREFTEWEIYQSSLAIQEVIFTLP | |
| | KDKLPEPILKEEWRAQWLSEHGLDTVPYKEAAGLNLIIKNAVN | |
| | TYKGVQVKVDNKNKNNLAKINRKNEIAKLNGEQEISFEEIKAF | |
| | DDKGYLLQRPSPNKSIYCYQSVSPKPFITSKYHNVNLPEEYIGY | |
| | YRKSNEPIVSPYQFDRLRIPIGEPGYVPKWQYTFLSKKENKRRK | |
| | LSKRIKNVSPILGIICIKKDWCVFDMRGLLRTNHWKKYHKPTD | |
| | SINDLFDYFTGDPVIDTKANVVRFRYKMENGIVNYKPVREKKG | |
| | KELLENICDQNGSCKLATVDVGQNNPVAIGLFELKKVNGELTK | |
| | TLISRHPTPIDFCNKITAYRERYDKLESSIKLDAIKQLTSEQKIEV | |
| | DNYNNNFTPQNTKQIVCSKLNINPNDLPWDKMISGTHFISEKA | |
| | QVSNKSEIYFTSTDKGKTKDVMKSDYKWFQDYKPKLSKEVRD | |
| | ALSDIEWRLRRESLEFNKLSKSREQDARQLANWISSMCDVIGIE | |
| | NLVKKNNFFGGSGKREPGWDNFYKPKKENRWWINAIHKALT | |
| | ELSQNKGKRVILLPAMRTSITCPKCKYCDSKNRNGEKFNCLKC | |
| | GIELNADIDVATENLATVAITAQSMPKPTCERSGDAKKPVRAR | |
| | KAKAPEFHDKLAPSYTVVLREAV | |
| CasPhi.12 | MIKPTVSQFLTPGFKLIRNHSRTAGLKLKNEGEEACKRFVREN | 57 |
| K38R | EIPKDECPNFQGGPAIANIIAKSREFTEWEIYQSSLAIQEVIFTLP | |
| | KDKLPEPILKEEWRAQWLSEHGLDTVPYKEAAGLNLIIKNAVN | |
| | TYKGVQVKVDNKNKNNLAKINRKNEIAKLNGEQEISFEEIKAF | |
| | DDKGYLLQKPSPNKSIYCYQSVSPKPFITSKYHNVNLPEEYIGY | |
| | YRKSNEPIVSPYQFDRLRIPIGEPGYVPKWQYTFLSKKENKRRK | |
| | LSKRIKNVSPILGIICIKKDWCVFDMRGLLRTNHWKKYHKPTD | |
| | SINDLFDYFTGDPVIDTKANVVRFRYKMENGIVNYKPVREKKG | |
| | KELLENICDQNGSCKLATVDVGQNNPVAIGLFELKKVNGELTK | |
| | TLISRHPTPIDFCNKITAYRERYDKLESSIKLDAIKQLTSEQKIEV | |
| | DNYNNNFTPQNTKQIVCSKLNINPNDLPWDKMISGTHFISEKA | |
| | QVSNKSEIYFTSTDKGKTKDVMKSDYKWFQDYKPKLSKEVRD | |
| | ALSDIEWRLRRESLEFNKLSKSREQDARQLANWISSMCDVIGIE | |
| | NLVKKNNFFGGSGKREPGWDNFYKPKKENRWWINAIHKALT | |
| | ELSQNKGKRVILLPAMRTSITCPKCKYCDSKNRNGEKFNCLKC | |
| | GIELNADIDVATENLATVAITAQSMPKPTCERSGDAKKPVRAR | |
| | KAKAPEFHDKLAPSYTVVLREAV | |
| CasPhi.12 | MIKPTVSQFLTPGFKLIRNHSRTAGLKLKNEGEEACKKFVREN | 58 |
| L182R | EIPKDECPNFQGGPAIANIIAKSREFTEWEIYQSSLAIQEVIFTLP | |
| | KDKLPEPILKEEWRAQWLSEHGLDTVPYKEAAGLNLIIKNAVN | |
| | TYKGVQVKVDNKNKNNLAKINRKNEIAKLNGEQEISFEEIKAF | |
| | DDKGYLRQKPSPNKSIYCYQSVSPKPFITSKYHNVNLPEEYIGY | |
| | YRKSNEPIVSPYQFDRLRIPIGEPGYVPKWQYTFLSKKENKRRK | |
| | LSKRIKNVSPILGIICIKKDWCVFDMRGLLRTNHWKKYHKPTD | |
| | SINDLFDYFTGDPVIDTKANVVRFRYKMENGIVNYKPVREKKG | |
| | KELLENICDQNGSCKLATVDVGQNNPVAIGLFELKKVNGELTK | |
| | TLISRHPTPIDFCNKITAYRERYDKLESSIKLDAIKQLTSEQKIEV | |
| | DNYNNNFTPQNTKQIVCSKLNINPNDLPWDKMISGTHFISEKA | |
| | QVSNKSEIYFTSTDKGKTKDVMKSDYKWFQDYKPKLSKEVRD | |
| | ALSDIEWRLRRESLEFNKLSKSREQDARQLANWISSMCDVIGIE | |
| | NLVKKNNFFGGSGKREPGWDNFYKPKKENRWWINAIHKALT | |
| | ELSQNKGKRVILLPAMRTSITCPKCKYCDSKNRNGEKFNCLKC | |
| | GIELNADIDVATENLATVAITAQSMPKPTCERSGDAKKPVRAR | |
| | KAKAPEFHDKLAPSYTVVLREAV | |
| CasPhi.12 | MIKPTVSQFLTPGFKLIRNHSRTAGLKLKNEGEEACKKFVREN | 59 |
| Q183R | EIPKDECPNFQGGPAIANIIAKSREFTEWEIYQSSLAIQEVIFTLP | |
| | KDKLPEPILKEEWRAQWLSEHGLDTVPYKEAAGLNLIIKNAVN | |
| | TYKGVQVKVDNKNKNNLAKINRKNEIAKLNGEQEISFEEIKAF | |
| | DDKGYLLRKPSPNKSIYCYQSVSPKPFITSKYHNVNLPEEYIGY | |
| | YRKSNEPIVSPYQFDRLRIPIGEPGYVPKWQYTFLSKKENKRRK | |
| | LSKRIKNVSPILGIICIKKDWCVFDMRGLLRTNHWKKYHKPTD | |
| | SINDLFDYFTGDPVIDTKANVVRFRYKMENGIVNYKPVREKKG | |
| | KELLENICDQNGSCKLATVDVGQNNPVAIGLFELKKVNGELTK | |
| | TLISRHPTPIDFCNKITAYRERYDKLESSIKLDAIKQLTSEQKIEV | |
| | DNYNNNFTPQNTKQIVCSKLNINPNDLPWDKMISGTHFISEKA | |
| | QVSNKSEIYFTSTDKGKTKDVMKSDYKWFQDYKPKLSKEVRD | |
| | ALSDIEWRLRRESLEFNKLSKSREQDARQLANWISSMCDVIGIE | |
| | NLVKKNNFFGGSGKREPGWDNFYKPKKENRWWINAIHKALT | |
| | ELSQNKGKRVILLPAMRTSITCPKCKYCDSKNRNGEKFNCLKC | |
| | GIELNADIDVATENLATVAITAQSMPKPTCERSGDAKKPVRAR | |
| | KAKAPEFHDKLAPSYTVVLREAV | |
| CasPhi.12 | MIKPTVSQFLTPGFKLIRNHSRTAGLKLKNEGEEACKKFVREN | 60 |
| S108R | EIPKDECPNFQGGPAIANIIAKSREFTEWEIYQSSLAIQEVIFTLP | |
| | KDKLPEPILKEEWRAQWLREHGLDTVPYKEAAGLNLIIKNAV | |
| | NTYKGVQVKVDNKNKNNLAKINRKNEIAKLNGEQEISFEEIKA | |
| | FDDKGYLLQKPSPNKSIYCYQSVSPKPFITSKYHNVNLPEEYIG | |
| | YYRKSNEPIVSPYQFDRLRIPIGEPGYVPKWQYTFLSKKENKRR | |
| | KLSKRIKNVSPILGIICIKKDWCVFDMRGLLRTNHWKKYHKPT | |
| | DSINDLFDYFTGDPVIDTKANVVRFRYKMENGIVNYKPVREKK | |
| | GKELLENICDQNGSCKLATVDVGQNNPVAIGLFELKKVNGELT | |
| | KTLISRHPTPIDFCNKITAYRERYDKLESSIKLDAIKQLTSEQKIE | |
| | VDNYNNNFTPQNTKQIVCSKLNINPNDLPWDKMISGTHFISEK | |
| | AQVSNKSEIYFTSTDKGKTKDVMKSDYKWFQDYKPKLSKEVR | |
| | DALSDIEWRLRRESLEFNKLSKSREQDARQLANWISSMCDVIGI | |
| | ENLVKKNNFFGGSGKREPGWDNFYKPKKENRWWINAIHKAL | |
| | TELSQNKGKRVILLPAMRTSITCPKCKYCDSKNRNGEKFNCLK | |
| | CGIELNADIDVATENLATVAITAQSMPKPTCERSGDAKKPVRA | |
| | RKAKAPEFHDKLAPSYTVVLREAV | |
| CasPhi.12 | MIKPTVSQFLTPGFKLIRNHSRTAGLKLKNEGEEACKKFVREN | 61 |
| S198R | EIPKDECPNFQGGPAIANIIAKSREFTEWEIYQSSLAIQEVIFTLP | |
| | KDKLPEPILKEEWRAQWLSEHGLDTVPYKEAAGLNLIIKNAVN | |
| | TYKGVQVKVDNKNKNNLAKINRKNEIAKLNGEQEISFEEIKAF | |
| | DDKGYLLQKPSPNKSIYCYQSVRPKPFITSKYHNVNLPEEYIGY | |
| | YRKSNEPIVSPYQFDRLRIPIGEPGYVPKWQYTFLSKKENKRRK | |
| | LSKRIKNVSPILGIICIKKDWCVFDMRGLLRTNHWKKYHKPTD | |
| | SINDLFDYFTGDPVIDTKANVVRFRYKMENGIVNYKPVREKKG | |
| | KELLENICDQNGSCKLATVDVGQNNPVAIGLFELKKVNGELTK | |
| | TLISRHPTPIDFCNKITAYRERYDKLESSIKLDAIKQLTSEQKIEV | |
| | DNYNNNFTPQNTKQIVCSKLNINPNDLPWDKMISGTHFISEKA | |
| | QVSNKSEIYFTSTDKGKTKDVMKSDYKWFQDYKPKLSKEVRD | |
| | ALSDIEWRLRRESLEFNKLSKSREQDARQLANWISSMCDVIGIE | |
| | NLVKKNNFFGGSGKREPGWDNFYKPKKENRWWINAIHKALT | |
| | ELSQNKGKRVILLPAMRTSITCPKCKYCDSKNRNGEKFNCLKC | |
| | GIELNADIDVATENLATVAITAQSMPKPTCERSGDAKKPVRAR | |
| | KAKAPEFHDKLAPSYTVVLREAV | |
| CasPhi.12 | MIKPTVSQFLTPGFKLIRNHSRTAGLKLKNEGEEACKKFVREN | 62 |
| T114R | EIPKDECPNFQGGPAIANIIAKSREFTEWEIYQSSLAIQEVIFTLP | |
| | KDKLPEPILKEEWRAQWLSEHGLDRVPYKEAAGLNLIIKNAV | |
| | NTYKGVQVKVDNKNKNNLAKINRKNEIAKLNGEQEISFEEIKA | |
| | FDDKGYLLQKPSPNKSIYCYQSVSPKPFITSKYHNVNLPEEYIG | |
| | YYRKSNEPIVSPYQFDRLRIPIGEPGYVPKWQYTFLSKKENKRR | |
| | KLSKRIKNVSPILGIICIKKDWCVFDMRGLLRTNHWKKYHKPT | |
| | DSINDLFDYFTGDPVIDTKANVVRFRYKMENGIVNYKPVREKK | |
| | GKELLENICDQNGSCKLATVDVGQNNPVAIGLFELKKVNGELT | |
| | KTLISRHPTPIDFCNKITAYRERYDKLESSIKLDAIKQLTSEQKIE | |
| | VDNYNNNFTPQNTKQIVCSKLNINPNDLPWDKMISGTHFISEK | |
| | AQVSNKSEIYFTSTDKGKTKDVMKSDYKWFQDYKPKLSKEVR | |
| | DALSDIEWRLRRESLEFNKLSKSREQDARQLANWISSMCDVIGI | |
| | ENLVKKNNFFGGSGKREPGWDNFYKPKKENRWWINAIHKAL | |
| | TELSQNKGKRVILLPAMRTSITCPKCKYCDSKNRNGEKFNCLK | |
| | CGIELNADIDVATENLATVAITAQSMPKPTCERSGDAKKPVRA | |
| | RKAKAPEFHDKLAPSYTVVLREAV | |
| CasPhi.12 | MIKPTVSQFLTPGFKLIRNHSRTAGLKLKNEGEEACKKFVREN | 63 |
| D369A | EIPKDECPNFQGGPAIANIIAKSREFTEWEIYQSSLAIQEVIFTLP | |
| | KDKLPEPILKEEWRAQWLSEHGLDTVPYKEAAGLNLIIKNAVN | |
| | TYKGVQVKVDNKNKNNLAKINRKNEIAKLNGEQEISFEEIKAF | |
| | DDKGYLLQKPSPNKSIYCYQSVSPKPFITSKYHNVNLPEEYIGY | |
| | YRKSNEPIVSPYQFDRLRIPIGEPGYVPKWQYTFLSKKENKRRK | |
| | LSKRIKNVSPILGIICIKKDWCVFDMRGLLRTNHWKKYHKPTD | |
| | SINDLFDYFTGDPVIDTKANVVRFRYKMENGIVNYKPVREKKG | |
| | KELLENICDQNGSCKLATVAVGQNNPVAIGLFELKKVNGELTK | |
| | TLISRHPTPIDFCNKITAYRERYDKLESSIKLDAIKQLTSEQKIEV | |
| | DNYNNNFTPQNTKQIVCSKLNINPNDLPWDKMISGTHFISEKA | |
| | QVSNKSEIYFTSTDKGKTKDVMKSDYKWFQDYKPKLSKEVRD | |
| | ALSDIEWRLRRESLEFNKLSKSREQDARQLANWISSMCDVIGIE | |
| | NLVKKNNFFGGSGKREPGWDNFYKPKKENRWWINAIHKALT | |
| | ELSQNKGKRVILLPAMRTSITCPKCKYCDSKNRNGEKFNCLKC | |
| | GIELNADIDVATENLATVAITAQSMPKPTCERSGDAKKPVRAR | |
| | KAKAPEFHDKLAPSYTVVLREAV | |
| CasPhi.12 | MIKPTVSQFLTPGFKLIRNHSRTAGLKLKNEGEEACKKFVREN | 64 |
| D369N | EIPKDECPNFQGGPAIANIIAKSREFTEWEIYQSSLAIQEVIFTLP | |
| | KDKLPEPILKEEWRAQWLSEHGLDTVPYKEAAGLNLIIKNAVN | |
| | TYKGVQVKVDNKNKNNLAKINRKNEIAKLNGEQEISFEEIKAF | |
| | DDKGYLLQKPSPNKSIYCYQSVSPKPFITSKYHNVNLPEEYIGY | |
| | YRKSNEPIVSPYQFDRLRIPIGEPGYVPKWQYTFLSKKENKRRK | |
| | LSKRIKNVSPILGIICIKKDWCVFDMRGLLRTNHWKKYHKPTD | |
| | SINDLFDYFTGDPVIDTKANVVRFRYKMENGIVNYKPVREKKG | |
| | KELLENICDQNGSCKLATVNVGQNNPVAIGLFELKKVNGELTK | |
| | TLISRHPTPIDFCNKITAYRERYDKLESSIKLDAIKQLTSEQKIEV | |
| | DNYNNNFTPQNTKQIVCSKLNINPNDLPWDKMISGTHFISEKA | |
| | QVSNKSEIYFTSTDKGKTKDVMKSDYKWFQDYKPKLSKEVRD | |
| | ALSDIEWRLRRESLEFNKLSKSREQDARQLANWISSMCDVIGIE | |
| | NLVKKNNFFGGSGKREPGWDNFYKPKKENRWWINAIHKALT | |
| | ELSQNKGKRVILLPAMRTSITCPKCKYCDSKNRNGEKFNCLKC | |
| | GIELNADIDVATENLATVAITAQSMPKPTCERSGDAKKPVRAR | |
| | KAKAPEFHDKLAPSYTVVLREAV | |
| CasPhi.12 | MIKPTVSQFLTPGFKLIRNHSRTAGLKLKNEGEEACKKFVREN | 65 |
| D658A | EIPKDECPNFQGGPAIANIIAKSREFTEWEIYQSSLAIQEVIFTLP | |
| | KDKLPEPILKEEWRAQWLSEHGLDTVPYKEAAGLNLIIKNAVN | |
| | TYKGVQVKVDNKNKNNLAKINRKNEIAKLNGEQEISFEEIKAF | |
| | DDKGYLLQKPSPNKSIYCYQSVSPKPFITSKYHNVNLPEEYIGY | |
| | YRKSNEPIVSPYQFDRLRIPIGEPGYVPKWQYTFLSKKENKRRK | |
| | LSKRIKNVSPILGIICIKKDWCVFDMRGLLRTNHWKKYHKPTD | |
| | SINDLFDYFTGDPVIDTKANVVRFRYKMENGIVNYKPVREKKG | |
| | KELLENICDQNGSCKLATVDVGQNNPVAIGLFELKKVNGELTK | |
| | TLISRHPTPIDFCNKITAYRERYDKLESSIKLDAIKQLTSEQKIEV | |
| | DNYNNNFTPQNTKQIVCSKLNINPNDLPWDKMISGTHFISEKA | |
| | QVSNKSEIYFTSTDKGKTKDVMKSDYKWFQDYKPKLSKEVRD | |
| | ALSDIEWRLRRESLEFNKLSKSREQDARQLANWISSMCDVIGIE | |
| | NLVKKNNFFGGSGKREPGWDNFYKPKKENRWWINAIHKALT | |
| | ELSQNKGKRVILLPAMRTSITCPKCKYCDSKNRNGEKFNCLKC | |
| | GIELNAAIDVATENLATVAITAQSMPKPTCERSGDAKKPVRAR | |
| | KAKAPEFHDKLAPSYTVVLREAV | |
| CasPhi.12 | MIKPTVSQFLTPGFKLIRNHSRTAGLKLKNEGEEACKKFVREN | 66 |
| D658N | EIPKDECPNFQGGPAIANIIAKSREFTEWEIYQSSLAIQEVIFTLP | |
| | KDKLPEPILKEEWRAQWLSEHGLDTVPYKEAAGLNLIIKNAVN | |
| | TYKGVQVKVDNKNKNNLAKINRKNEIAKLNGEQEISFEEIKAF | |
| | DDKGYLLQKPSPNKSIYCYQSVSPKPFITSKYHNVNLPEEYIGY | |
| | YRKSNEPIVSPYQFDRLRIPIGEPGYVPKWQYTFLSKKENKRRK | |
| | LSKRIKNVSPILGIICIKKDWCVFDMRGLLRTNHWKKYHKPTD | |
| | SINDLFDYFTGDPVIDTKANVVRFRYKMENGIVNYKPVREKKG | |
| | KELLENICDQNGSCKLATVDVGQNNPVAIGLFELKKVNGELTK | |
| | TLISRHPTPIDFCNKITAYRERYDKLESSIKLDAIKQLTSEQKIEV | |
| | DNYNNNFTPQNTKQIVCSKLNINPNDLPWDKMISGTHFISEKA | |
| | QVSNKSEIYFTSTDKGKTKDVMKSDYKWFQDYKPKLSKEVRD | |
| | ALSDIEWRLRRESLEFNKLSKSREQDARQLANWISSMCDVIGIE | |
| | NLVKKNNFFGGSGKREPGWDNFYKPKKENRWWINAIHKALT | |
| | ELSQNKGKRVILLPAMRTSITCPKCKYCDSKNRNGEKFNCLKC | |
| | GIELNANIDVATENLATVAITAQSMPKPTCERSGDAKKPVRAR | |
| | KAKAPEFHDKLAPSYTVVLREAV | |
| CasPhi | MIKPTVSQFLTPGFKLIRNHSRTAGLKLKNEGEEACKKFVREN | 203 |
| j12_L17_ | EIPKDECPNFQGGPAIANIIAKSREFTEWEIYQSSLAIQEVIFTLP | |
| 18_del1 | KDKLPEPILKEEWRAQWLSEHGLDTVPYKEAAGLNLIIKNAVN | |
| | TYKGVQVKVDNKNKNNLAKINRKNEIAKLNGEQEISFEEIKAF | |
| | DDKGYLLQKPSPNKSIYCYQSVSPKPFITSKYHNVNLPEEYIGY | |
| | YRKSNEPIVSPYQFDRLRIPIGEPGYVPKWQYTFLSKKENKRRK | |
| | LSKRIKNVSPILGIICIKKDWCVFDMRGLLRTNHWKKYHKPTD | |
| | SINDLFDYFTGDPVIDTKANVVRFRYKMENGIVNYKPVREKKG | |
| | KELLENICDQNGSCKLATVDVGQNNPVAIGLFELKKVNGELTK | |
| | TLISRHPTPIDFCNKITAYRERYDKLESSIKLDAIKQLTSEQKIEV | |
| | DNYNNNFTPQNTKQIVCSKLNINPNDLPWDKMISGTHFISEKA | |
| | QGSSGDYKWFQDYKPKLSKEVRDALSDIEWRLRRESLEFNKL | |
| | SKSREQDARQLANWISSMCDVIGIENLVKKNNFFGGSGKREPG | |
| | WDNFYKPKKENRWWINAIHKALTELSQNKGKRVILLPAMRTS | |
| | ITCPKCKYCDSKNRNGEKFNCLKCGIELNADIDVATENLATVA | |
| | ITAQSMPKPTCERSGDAKKPVRARKAKAPEFHDKLAPSYTVVL | |
| | REAV | |
| CasPhi | MIKPTVSQFLTPGFKLIRNHSRTAGLKLKNEGEEACKKFVREN | 204 |
| j12_L17_ | EIPKDECPNFQGGPAIANIIAKSREFTEWEIYQSSLAIQEVIFTLP | |
| 18_del2 | KDKLPEPILKEEWRAQWLSEHGLDTVPYKEAAGLNLIIKNAVN | |
| | TYKGVQVKVDNKNKNNLAKINRKNEIAKLNGEQEISFEEIKAF | |
| | DDKGYLLQKPSPNKSIYCYQSVSPKPFITSKYHNVNLPEEYIGY | |
| | YRKSNEPIVSPYQFDRLRIPIGEPGYVPKWQYTFLSKKENKRRK | |
| | LSKRIKNVSPILGIICIKKDWCVFDMRGLLRTNHWKKYHKPTD | |
| | SINDLFDYFTGDPVIDTKANVVRFRYKMENGIVNYKPVREKKG | |
| | KELLENICDQNGSCKLATVDVGQNNPVAIGLFELKKVNGELTK | |
| | TLISRHPTPIDFCNKITAYRERYDKLESSIKLDAIKQLTSEQKIEV | |
| | DNYNNNFTPQNTKQIVCSKLNINPNDLPWDKMISGTHFISEKA | |
| | QVSNKSEGSSGDYKWFQDYKPKLSKEVRDALSDIEWRLRRES | |
| | LEFNKLSKSREQDARQLANWISSMCDVIGIENLVKKNNFFGGS | |
| | GKREPGWDNFYKPKKENRWWINAIHKALTELSQNKGKRVILL | |
| | PAMRTSITCPKCKYCDSKNRNGEKFNCLKCGIELNADIDVATE | |
| | NLATVAITAQSMPKPTCERSGDAKKPVRARKAKAPEFHDKLA | |
| | PSYTVVLREAV | |
| CasPhi.12- | MIKPTVSQFLTPGFKLIRNHSRTAGKKLKNEGEEACKKFVREN | 794 |
| L26K- | EIPKDECPNFQGGPAIANIIAKSREFTEWEIYQSSLAIQEVIFTLP | |
| E567Q | KDKLPEPILKEEWRAQWLSEHGLDTVPYKEAAGLNLIIKNAVN | |
| | TYKGVQVKVDNKNKNNLAKINRKNEIAKLNGEQEISFEEIKAF | |
| | DDKGYLLQKPSPNKSIYCYQSVSPKPFITSKYHNVNLPEEYIGY | |
| | YRKSNEPIVSPYQFDRLRIPIGEPGYVPKWQYTFLSKKENKRRK | |
| | LSKRIKNVSPILGIICIKKDWCVFDMRGLLRTNHWKKYHKPTD | |
| | SINDLFDYFTGDPVIDTKANVVRFRYKMENGIVNYKPVREKKG | |
| | KELLENICDQNGSCKLATVDVGQNNPVAIGLFELKKVNGELTK | |
| | TLISRHPTPIDFCNKITAYRERYDKLESSIKLDAIKQLTSEQKIEV | |
| | DNYNNNFTPQNTKQIVCSKLNINPNDLPWDKMISGTHFISEKA | |
| | QVSNKSEIYFTSTDKGKTKDVMKSDYKWFQDYKPKLSKEVRD | |
| | ALSDIEWRLRRESLEFNKLSKSREQDARQLANWISSMCDVIGIQ | |
| | NLVKKNNFFGGSGKREPGWDNFYKPKKENRWWINAIHKALT | |
| | ELSQNKGKRVILLPAMRTSITCPKCKYCDSKNRNGEKFNCLKC | |
| | GIELNADIDVATENLATVAITAQSMPKPTCERSGDAKKPVRAR | |
| | KAKAPEFHDKLAPSYTVVLREAV | |
| CasPhi.12- | MIKPTVSQFLTPGFKLIRNHSRTAGRKLKNEGEEACKKFVREN | 2090 |
| L26R- | EIPKDECPNFQGGPAIANIIAKSREFTEWEIYQSSLAIQEVIFTLP | |
| I471T | KDKLPEPILKEEWRAQWLSEHGLDTVPYKEAAGLNLIIKNAVN | |
| | TYKGVQVKVDNKNKNNLAKINRKNEIAKLNGEQEISFEEIKAF | |
| | DDKGYLLQKPSPNKSIYCYQSVSPKPFITSKYHNVNLPEEYIGY | |
| | YRKSNEPIVSPYQFDRLRIPIGEPGYVPKWQYTFLSKKENKRRK | |
| | LSKRIKNVSPILGIICIKKDWCVFDMRGLLRTNHWKKYHKPTD | |
| | SINDLFDYFTGDPVIDTKANVVRFRYKMENGIVNYKPVREKKG | |
| | KELLENICDQNGSCKLATVDVGQNNPVAIGLFELKKVNGELTK | |
| | TLISRHPTPIDFCNKITAYRERYDKLESSIKLDAIKQLTSEQKIEV | |
| | DNYNNNFTPQNTKQIVCSKLNINPNDLPWDKMTSGTHFISEKA | |
| | QVSNKSEIYFTSTDKGKTKDVMKSDYKWFQDYKPKLSKEVRD | |
| | ALSDIEWRLRRESLEFNKLSKSREQDARQLANWISSMCDVIGIE | |
| | NLVKKNNFFGGSGKREPGWDNFYKPKKENRWWINAIHKALT | |
| | ELSQNKGKRVILLPAMRTSITCPKCKYCDSKNRNGEKFNCLKC | |
| | GIELNADIDVATENLATVAITAQSMPKPTCERSGDAKKPVRAR | |
| | KAKAPEFHDKLAPSYTVVLREAV | |
| CasPhi.12- | MIKPTVSQFLTPGFKLIRNHSRTAGLKLKNEGEEACKKFVREN | 2091 |
| I471T | EIPKDECPNFQGGPAIANIIAKSREFTEWEIYQSSLAIQEVIFTLP | |
| | KDKLPEPILKEEWRAQWLSEHGLDTVPYKEAAGLNLIIKNAVN | |
| | TYKGVQVKVDNKNKNNLAKINRKNEIAKLNGEQEISFEEIKAF | |
| | DDKGYLLQKPSPNKSIYCYQSVSPKPFITSKYHNVNLPEEYIGY | |
| | YRKSNEPIVSPYQFDRLRIPIGEPGYVPKWQYTFLSKKENKRRK | |
| | LSKRIKNVSPILGIICIKKDWCVFDMRGLLRTNHWKKYHKPTD | |
| | SINDLFDYFTGDPVIDTKANVVRFRYKMENGIVNYKPVREKKG | |
| | KELLENICDQNGSCKLATVDVGQNNPVAIGLFELKKVNGELTK | |
| | TLISRHPTPIDFCNKITAYRERYDKLESSIKLDAIKQLTSEQKIEV | |
| | DNYNNNFTPQNTKQIVCSKLNINPNDLPWDKMTSGTHFISEKA | |
| | QVSNKSEIYFTSTDKGKTKDVMKSDYKWFQDYKPKLSKEVRD | |
| | ALSDIEWRLRRESLEFNKLSKSREQDARQLANWISSMCDVIGIE | |
| | NLVKKNNFFGGSGKREPGWDNFYKPKKENRWWINAIHKALT | |
| | ELSQNKGKRVILLPAMRTSITCPKCKYCDSKNRNGEKFNCLKC | |
| | GIELNADIDVATENLATVAITAQSMPKPTCERSGDAKKPVRAR | |
| | KAKAPEFHDKLAPSYTVVLREAV | |

TABLE 19
Exemplary Amino Acid Alterations Relative to SEQ ID NO: 32

| Effects | Amino Acid Alterations |
|---|---|
| | At least one substitution (i.e., with R, K or H) selected from I2, T5, K15, |
| | R18, H20, S21, L26, N30, E33, E34, A35, K37, K38, R41, N43, Q54, |
| | Q79R, K92E, K99R, S108, E109, H110, G111, D113, T114, P116, K118, |
| | E119, A121, N132, K135, Q138, V139, N148, L149, E157, E164, E166, |
| | E170, Y180, L182, Q183, K184, S186, K189, S196, S198, K200, I203, |
| | S205, K206, Y207, H208, N209, Y220, S223, E258, K281, K348, N355, |
| | S362, N406, K435, I471, I489, Y490, F491, D495, K496, K498, K500, |
| | D501, V502, K504, S505, D506, V521, E567, N568, S579, Q612, S638, |
| | F701, and P707 |
| Enhanced nuclease | T5R, L26R, L26K, A121Q, N148R, V139R, S198R, H208R, S223P, |
| activity relative to | E258K, N355R, I471T, S579R, F701R, P707R, K189P, S638K, Q54R, |
| the wild-type | Q79R, Y220S, N406K, E119S, K92E, K435Q, N568D, and V521T |
| effector protein | Double mutations: L26K/A121Q, L26X/A121Q, K99R/L149R, |
| | K99R/N148R, L149R/H208R, S362R/L26X L26X/N148R, L26X/H208R, |
| | N30R/N148R, L26X/K99R, L26X/P707R, L26X/L149R, L26X/N30R, |
| | L26X/N355R, L26X/K281R, L26X/S108R, L26X/K348R, T5R/V139R, |
| | I2R/V139R, K99R/S186R, L26X/A673G, L26X/Q674R, S579R/L26K, |
| | F701R/E258K, T5R/L26K, L26X/K435Q, L26X/G685R, L26X/Q674K, |
| | L26X/P699R, L26X/T70E, L26X/Q232R, L26X/T252R, L26X/E567Q, |
| | L26X/P679R, L26X/E83K, L26X/E73P, L26X/K248E, L26X, |
| | T5R/S223P, S579R/S223P, L26X/S223P, T5R/A121Q, L26X/A696R, |
| | S198R/I471T, L26X/N153R, L26X/E682R, L26X/D703R, Q612R/L26K, |
| | L26X/I471T, K348R/L26K, S579R/I471T, L26X/V228R, |
| | T5R/S638K, S579R/K189P, S579R/E258K, L26X/K260R, L26X/S638K, |
| | S579R/Y220S, T5R/I471T, L26X/F233R, L26X/V521T, F701R/A121Q, |
| | L26X/G361R, S198R/E258K, L26X/S472R, T5R/Y220S, L26X/A150K, |
| | L26X/S684R, L26X/B157R, L26X/K248R, F701R/L26K, S198R/N406K, |
| | S198R/Y220S, S198R/S638K, S198R/V521T, S579R/A121Q, |
| | K348R/Y220S, S198R/K189P, L26X/E242R, L26X/K678R, |
| | T5R/N406K, L26X/1158K, T5R/V521T, L26X/N259R, L26X/K257R, |
| | L26X/K256R, T5R/K189P, L26X/C405R, S579R/V521T, S579R/N406K, |
| | T5R/K92E, TSR/I258K, L26X/I97R, S579R/S638K, T5R/K435Q, |
| | F701R/S638K, L26X/L236R, F701R/I471T, Q612R/S223P, |
| | F701R/S223P, S198R/E119S, S579R/K92E, L26X/E715R, |
| | Q612R/I471T, F701R/Y220S, S198R/S223P, and L26X/K266R, wherein |
| | X is selected from R and K. |
| Nickase activity | E157A, E164A, E164L, E166A, E1661, E170A, I489A, I489S, Y490S, |
| | Y490A, F491A, F491S, F491G, D495G, D495R, D495K, K496A, |
| | K496S, K498A, K498S, K500A, K500S, D501R, D501G, D501K, |
| | V502A, V502S, K504A, K504S, S505R, D506A; |
| | deletion of S478-S505 of SEQ ID NO: 32; |
| | deletion of S478-S505 of SEQ ID NO: 32 and insertion of the sequence of |
| | SDLYIERGGDPRDVHQQVETKPKGKRKSEIRILKIR (SEQ ID NO: |
| | 205); |
| | deletion of S478-S505 of SEQ ID NO: 32 and insertion of the sequence of |
| | SDYIVDHGGDPEKVFFETKSKKDKTKRYKRR (SEQ ID NO: 206); |
| | an amino acid sequence that is at least 90%, at least 95%, at least 97%, at |
| | least 98%, at least 99% identical, or is 100% identical to SEQ ID NO: |
| | 203; |
| | an amino acid sequence that is at least 90%, at least 95%, at least 97%, at |
| | least 98%, at least 99% identical, or is 100% identical to SEQ ID NO: |
| | 204 |
| Reduced or | D369A, D369N, D658A, D658N, E567A, E567Q |
| abolished nuclease | |
| activity relative to | |
| the wild-type | |
| effector protein | |

In certain embodiments, compositions comprise an effector protein an engineered guide nucleic acid, wherein the amino acid sequence of the effector protein comprises at least about 200, at least about 220, at least about 240, at least about 260, at least about 280, at least about 300, at least about 320, at least about 340, at least about 360, at least about 380, at least about 400, at least about 420, at least about 440, at least about 460, at least about 480, at least about 500, at least about 520, at least about 540, at least about 560, at least about 580, at least about 600, at least about 620, at least about 640, at least about 660, at least about 680, at least about 700, or at least about 717 contiguous amino acids or more of any one of the sequences as set forth in TABLES 15-19. In certain embodiments, compositions comprise an effector protein and an engineered guide nucleic acid, wherein the amino acid sequence of the effector protein comprises at least about 200 contiguous amino acids or more of any one of the sequences as set forth in TABLES 15-19. In certain embodiments, compositions comprise an effector protein and an engineered guide nucleic acid, wherein the amino acid sequence of the effector protein comprises at least about 300 contiguous amino acids or more of any one of the sequences as set forth in TABLES 15-19. In certain embodiments, compositions comprise an effector protein and an engineered guide nucleic acid, wherein the amino acid sequence of the effector protein comprises at least about 700 contiguous amino acids or more of any one of the sequences as set forth in TABLES 15-19.

In some embodiments, compositions, systems, and methods described herein comprise an effector protein or a nucleic acid encoding the effector protein, wherein the effector protein comprises one or more amino acid alterations relative to the sequence recited in TABLES 15-19. In some embodiments, the effector protein comprising one or more amino acid alterations is a variant of an effector protein described herein. It is understood that any reference to an effector protein herein also refers to an effector protein variant as described herein. In some embodiments, an amino acid alteration comprises a deletion of an amino acid. In some embodiments, an amino acid alteration comprises an insertion of an amino acid. In some embodiments, an amino acid alteration comprises a conservative amino acid substitution. In some embodiments, an amino acid alteration comprises a non-conservative amino acid substitution. In some embodiments, one or more amino acid alterations comprises a combination of one or more conservative amino acid substitutions and one or more non-conservative amino acid substitutions. When describing a conservative amino acid substitution herein, reference is made to the replacement of one amino acid for another such that the replacement takes place within a family of amino acids that are related in their side chains. Conversely, when describing a non-conservative alteration (e.g., non-conservative substitution), reference is made to the replacement of one amino acid residue for another that does not have a related side chain. It is understood that genetically encoded amino acids can be divided into four families having related side chains: (1) acidic (negatively charged): Asp (D), Glu (E); (2) basic (positively charged): Lys (K), Arg (R), His (H); (3) non-polar (hydrophobic): Cys (C), Ala (A), Val (V), Leu (L), Ile (I), Pro (P), Phe (F), Met (M), Trp (W), Gly (G), Tyr (Y), with non-polar also being subdivided into: (i) strongly hydrophobic: Ala (A), Val (V), Leu (L), Ile (I), Met (M), Phe (F); and (ii) moderately hydrophobic: Gly (G), Pro (P), Cys (C), Tyr (Y), Trp (W); and (4) uncharged polar: Asn (N), Gln (Q), Ser (S), Thr (T). Amino acids may be related by aliphatic side chains: Gly (G), Ala (A), Val (V), Leu (L), Ile (I), Ser (S), Thr (T), with Ser (S) and Thr (T) optionally being grouped separately as aliphatic-hydroxyl. Amino acids may be related by aromatic side chains: Phe (F), Tyr (Y), Trp (W). Amino acids may be related by amide side chains: Asn (N), Gln (Q). Amino acids may be related by sulfur-containing side chains: Cys (C) and Met (M).

In some embodiments, an effector protein comprises an amino acid sequence that is at least 65%, at least 70%, at least 75%, at least 80%, at least 85%, at least 90%, at least 95%, at least 97%, or at least 98%, at least 99%, or 100% identical to a sequence selected from TABLES 15-19, wherein the effector protein comprises 1, 2, 3, 4, 5, 6, 7, 8, 9 or 10 conservative amino acid substitutions relative to the sequence selected from TABLES 15-19. In some embodiments, an effector protein comprises an amino acid sequence that is at least 65%, at least 70%, at least 75%, at least 80%, at least 85%, at least 90%, at least 95%, at least 97%, or at least 98%, at least 99%, or 100% identical to a sequence selected from TABLES 15-19, wherein the effector protein comprises 1 to 10, 10 to 20, 20 to 30, or 30 to 40 conservative amino acid substitutions relative to the sequence selected from TABLES 15-19. In some embodiments, an effector protein comprises an amino acid sequence that is at least 65%, at least 70%, at least 75%, at least 80%, at least 85%, at least 90%, at least 95%, at least 97%, or at least 98%, at least 99%, or 100% identical to a sequence selected from TABLES 15-19, wherein the effector protein comprises not more than 1, 2, 3, 4, 5, 6, 7, 8, 9 or 10 non-conservative amino acid substitutions relative to the sequence selected from TABLES 15-19.

In certain embodiments, compositions, systems, and methods described herein comprise an effector protein, or a nucleic acid encoding the effector protein, wherein the effector protein comprises an amino acid sequence that is at least 65%, at least 70%, at least 75%, at least 80%, at least 85%, at least 90%, at least 95%, at least 98%, at least 99%, or 100% similar to any one of the sequences selected from TABLES 15-19. An amino acid sequence of the effector protein is similar to the reference amino acid sequence, when a value that is calculated by dividing a similarity score by the length of the alignment. The similarity of two amino acid sequences can be calculated by using a BLOSUM62 similarity matrix (Henikoff and Henikoff, Proc. Natl. Acad. Sci. USA., 89:10915-10919 (1992)) that is transformed so that any value ≥1 is replaced with +1 and any value ≤0 is replaced with 0. For example, an Ile (I) to Leu (L) substitution is scored at +2.0 by the BLOSUM62 similarity matrix, which in the transformed matrix is scored at +1. This transformation allows the calculation of percent similarity, rather than a similarity score. Alternately, when comparing two full protein sequences, the proteins can be aligned using pairwise MUSCLE alignment. Then, the % similarity can be scored at each residue and divided by the length of the alignment. For determining % similarity over a protein domain or motif, a multilevel consensus sequence (or PROSITE motif sequence) can be used to identify how strongly each domain or motif is conserved. In calculating the similarity of a domain or motif, the second and third levels of the multilevel sequence are treated as equivalent to the top level. Additionally, if a substitution could be treated as conservative with any of the amino acids in that position of the multilevel consensus sequence, +1 point is assigned. For example, given the multilevel consensus sequence: RLG and YCK, the test sequence QIq would receive three points. This is because in the transformed BLOSUM62 matrix, each combination is scored as: Q-R: +1; Q-Y: +0; I-L: +1; I-C: +0; Q-G: +0; Q-K: +1. For each position, the highest score is used when calculating similarity. The % similarity can also be calculated using commercially available programs, such as the Geneious Prime software given the parameters matrix=BLOSUM62 and threshold ≥1.

In some cases, the effector proteins comprise a RuvC domain. In some embodiments, the RuvC domain may be defined by a single, contiguous sequence, or a set of RuvC subdomains that are not contiguous with respect to the primary amino acid sequence of the protein. An effector protein of the present disclosure may include multiple RuvC subdomains, which may combine to generate a RuvC domain with substrate binding or catalytic activity. For example, an effector protein may include three RuvC subdomains (RuvC-I, RuvC-II, and RuvC-III) that are not contiguous with respect to the primary amino acid sequence of the effector protein but form a RuvC domain once the protein is produced and folds. In many cases, effector proteins comprise a recognition domain with a binding affinity for a guide nucleic acid or for a guide nucleic acid-target nucleic acid heteroduplex. An effector protein may comprise a zinc finger domain.

An effector protein may be small, which may be beneficial for nucleic acid detection or editing (for example, the effector protein may be less likely to adsorb to a surface or another biological species due to its small size). The smaller nature of these effector proteins may allow for them to be more easily packaged and delivered with higher efficiency in the context of genome editing and more readily incorporated as a reagent in an assay. In some embodiments, the length of the effector protein is less than 400 linked amino acid residues. In some embodiments, the length of the effector protein is less than 425 linked amino acid residues. In some embodiments, the length of the effector protein is less than 450 linked amino acid residues. In some embodiments, the length of the effector protein is less than 475 linked amino acid residues. In some embodiments, the length of the effector protein is less than 500 linked amino acid residues. In some embodiments, the length of the effector protein is less than 550, less than 600, less than 650, less than 700, or less than 717 linked amino acid residues. In some embodiments, the length of the effector protein is less than 500 linked amino acid residues. In some embodiments, the length of the effector protein is about 400 to about 717 linked amino acids. In some embodiments, the length of the effector protein is about 400 to about 700 linked amino acid residues. In some embodiments, the length of the effector protein is about 650 to about 675 linked amino acids.

In some embodiments, an effect protein is encoded by a nucleic acid sequence that is at least 65%, at least 70%, at least 75%, at least 80%, at least 85%, at least 90%, at least 95%, at least 97%, or at least 98%, or at least 99%, identical to SEQ ID NO: 2092. In some embodiments, the nucleic acid sequence comprises one or more untranslateable regions (UTR), one or more nuclear localization regions, one or more stop codons, and or more adenine bases that are encompassed in a polyA tail. An exemplary nucleic acid sequence is shown in TABLE 20 below (bold=UTR11s, italics=NLS sequences, bold underlined=stop codons, and series of As=polyA tail):

TABLE 20
Exemplary Nuclease mRNA (CasPhi.12 L26R, 1471T)

mRNA Sequence
AGGGUCCCGCAGUCGGCGUCCAGCGGCUCUGCUUGUUCGUGUGUGUGU
CGUUGCAGGCCUUAUUCGGAUCCGCCACCAUGGCCCCAAAGAAGAAGC
GGAAGGUCGGUAUCCACGGAGUCCCAGCAGCCAUCAAACCUACCGUGA
GCCAGUUCCUGACCCCUGGCUUCAAGCUGAUCCGGAACCACAGCCGGA
CCGCUGGCAGAAAGCUUAAGAAUGAAGGCGAGGAAGCCUGCAAAAAGU
UCGUGAGAGAAAACGAGAUCCCUAAGGACGAGUGCCCCAACUUCCAGG
GCGGCCCUGCCAUCGCUAAUAUUAUCGCCAAGAGCAGAGAGUUCACCG
AGUGGGAGAUCUAUCAGAGCAGCCUGGCCAUUCAGGAGGUGAUCUUCA
CGCUGCCAAAGGACAAGCUGCCCGAGCCAAUACUGAAGGAAGAGUGGC
GGGCCCAGUGGCUGUCCGAGCACGGCCUGGAUACCGUGCCAUACAAGG
AGGCCGCGGGCCUGAACCUGAUCAUCAAGAACGCCGUAAACACCUACA
AGGGCGUGCAGGUGAAGGUGGAUAACAAAAACAAGAACAACCUGGCAA
AAAUCAACAGAAAGAACGAGAUCGCCAAGCUGAAUGGCGAGCAGGAGA
UCAGCUUUGAGGAAAUCAAAGCCUUCGACGAUAAGGGCUACCUGCUGC
AGAAACCAUCUCCUAACAAGAGCAUCUAUUGUUACCAGAGCGUGAGCC
CCAAGCCUUUCAUCACAAGCAAGUACCACAACGUGAACCUGCCCGAGG
AGUACAUCGGCUACUACAGAAAGUCUAAUGAGCCUAUCGUGAGCCCUU
ACCAGUUUGACAGACUGCGGAUCCCUAUCGGCGAACCCGGAUAUGUGC
CUAAGUGGCAGUACACCUUCCUGUCUAAAAAGGAAAACAAGCGGAGAA
AGCUGUCCAAGCGCAUCAAGAACGUGAGCCCCAUCCUGGGCAUCAUCU
GUAUCAAGAAAGACUGGUGCGUUUUUGACAUGCGCGGCCUGCUGAGAA
CAAACCACUGGAAGAAGUACCAUAAGCCUACAGAUAGCAUUAACGACC
UGUUUGAUUAUUUCACCGGCGACCCUGUGAUCGACACAAAGGCCAACG
UCGUGAGAUUCAGGUACAAGAUGGAAAAUGGAAUCGUGAACUACAAGC
CAGUCAGAGAGAAGAAGGGCAAGGAACUGCUGGAAAAUAUCUGCGAUC
AGAACGGUAGCUGCAAGCUGGCCACAGUGGACGUGGGCCAGAACAACC
CCGUGGCCAUCGGACUGUUCGAGCUCAAGAAGGUCAAUGGUGAGCUGA
CCAAGACACUGAUCUCUCGGCACCCCACACCUAUCGACUUCUGUAACA
AAAUCACCGCCUACCGGGAGAGAUACGACAAGCUGGAAAGCAGCAUCA
AACUCGACGCCAUCAAGCAGCUGACAUCUGAGCAGAAGAUCGAGGUGG
ACAACUACAACAAUAACUUCACCCCUCAGAACACCAAGCAAAUCGUGU
GCAGCAAGCUGAACAUUAACCCUAACGAUCUGCCUUGGGAUAAGAUGA
CCAGCGGCACACACUUCAUCUCUGAAAAGGCUCAGGUGAGCAACAAGU
CUGAAAUCUACUUUACCAGCACCGACAAGGGCAAGACCAAAGACGUGA
UGAAGUCCGAUUACAAGUGGUUCCAAGACUACAAGCCUAAACUGAGCA
AAGAGGUGAGAGAUGCCCUGAGCGACAUUGAGUGGCGCCUGCGGCGGG
AAUCUCUGGAAUUUAACAAACUUUCCAAGAGCAGAGAGCAAGACGCUA
GACAGCUGGCCAAUUGGAUCAGCAGCAUGUGCGAUGUGAUCGGCAUCG
AGAACCUGGUGAAGAAAAACAAUUUUUUCGGCGGAUCUGGCAAACGGG
AACCUGGAUGGGACAACUUCUACAAGCCCAAAAAGGAAAACAGAUGGU
GGAUCAACGCCAUCCACAAAGCCCUGACCGAGCUGAGCCAGAACAAGG
GCAAGAGAGUGAUCCUGCUGCCCGCCAUGAGAACCAGCAUCACCUGCC
CUAAAUGCAAGUACUGCGACAGCAAAAACAGAAACGGCGAAAAGUUCA
AUUGCCUGAAGUGUGGAAUCGAGCUGAACGCUGACAUCGAUGUGGCAA
CCGAAAACCUGGCUACAGUUGCCAUCACCGCCCAAUCCAUGCCUAAAC
CCACAUGUGAAAGGUCCGGCGACGCCAAGAAGCCUGUGAGAGCCAGAA
AGGCCAAGGCUCCUGAGUUCCACGACAAACUGGCCCCUAGCUACACCG
UGGUGCUGAGAGAGGCCGUGAAAAGGCCGGCGGCCACGAAAAAGGCCG
GCCAGGCAAAAAAGAAAAAGUAAUGACCAUCACAUUUAAAAGCAUCUC
AGCCUACCAUGAGAAUAAGAGAAAGAAAAUGAAGAUCAAUAGCUUAUU
CAUCUCUUUUUCUUUUUCGUUGGUGUAAAGCCAACACCCUGUCUAAAA
AACAUAAAUUUCUUUAAUCAUUUUGCCUCUUUUCUCUGUGCUUCAAUU
AAUAAAAAAUGGAAAGAACCUCGAGAAAAAAAAAAAAAAAAAAAAAAA
AAAAAAAAAAAAAAAAAAAAAAAAAAAAAAAAAAAAAAAAAAAAAAAA
AAAAAAAAAAAAAAAAAAAAAAAAAAAAAAAAAAAAAAAAAAAAAAAA
A
(SEQ ID NO: 2092)

### Protospacer Adjacent Motif (PAM) Sequences

Effector proteins of the present disclosure, dimers thereof, and multimeric complexes thereof may cleave or nick a target nucleic acid within or near a protospacer adjacent motif (PAM) sequence of the target nucleic acid. In some embodiments, cleavage occurs within 10, 20, 30, 40 or 50 nucleotides of a 5′ or 3′ terminus of a PAM sequence. In some embodiments, cleavage occurs within 1, 2, 3, 4, 5, 6, 7, 8, 9 or 10 nucleotides of a 5′ or 3′ terminus of a PAM sequence. A target nucleic acid may comprise a PAM sequence adjacent to a target sequence. In some embodiments, systems, compositions, and methods comprise a guide nucleic acid or use thereof, wherein the guide nucleic acid comprises a spacer sequence that is complementary to a target sequence that is adjacent to a PAM sequence.

In some embodiments, systems, compositions, and methods comprise a guide nucleic acid or use thereof, wherein the guide nucleic acid comprises a spacer sequence that is complementary to a target sequence that is adjacent to a PAM sequence. A target nucleic acid may comprise a PAM sequence adjacent to a target sequence.

In some embodiments, the PAM is 5′-NTTN-3′, wherein N=any nucleic acid. Exemplary PAM sequences are disclosed in TABLE 21. In some embodiments, the effector protein recognizes a PAM sequence comprising any of the following nucleotide sequences as set forth in TABLE 21. In some embodiments, the effector protein comprises an amino acid sequence that is at least 65%, at least 70%, at least 75%, at least 80%, at least 85%, at least 90%, at least 95%, at least 97%, or at least 98%, at least 99%, or 100% identical to a sequence selected from TABLES 15, 18, and 19. In some embodiments, the effector protein comprises an amino acid sequence that is at least 65%, at least 70%, at least 75%, at least 80%, at least 85%, at least 90%, at least 95%, at least 97%, or at least 98%, at least 99%, or 100% identical to SEQ ID NO: 32.

TABLE 21
Exemplary PAM NTTN Sequences

| PAM # | PAM Sequence (5′-3′) |
|---|---|
| 1 | NTTG |
| 2 | NTTC |
| 3 | NTTT |
| 4 | NTTA |

In some embodiments, the PAM is 5′-NNTN-3′, wherein N=any nucleic acid. In some embodiments, the PAM is 5′-TNTR-3′, wherein N=any nucleic acid and wherein R=a purine nucleic acid (i.e., A or G). Non-limiting examples of TNTR PAM sequences are disclosed in TABLE 22. In some embodiments, the PAM is 5′-TNTG-3.′ In some embodiments, the effector protein recognizes a PAM sequence comprising any of the following nucleotide sequences as set forth in TABLE 22. In some embodiments, the effector protein comprises an amino acid sequence that is at least 65%, at least 70%, at least 75%, at least 80%, at least 85%, at least 90%, at least 95%, at least 97%, or at least 98%, at least 99%, or 100% identical to a sequence selected from TABLES 15-17. In some embodiments, the effector protein comprises an amino acid sequence that is at least 65%, at least 70%, at least 75%, at least 80%, at least 85%, at least 90%, at least 95%, at least 97%, or at least 98%, at least 99%, or 100% identical to SEQ ID NO: 773.

TABLE 22
Exemplary PAM TNTR Sequences

| PAM # | PAM Sequence (5′-3′) |
|---|---|
| 1 | TTTG |
| 2 | TCTG |
| 3 | TGTG |
| 4 | TCTA |
| 5 | TATA |
| 6 | TTTA |
| 7 | TGTA |
| 8 | TATG |

Non-limiting examples of NNTN PAMs include GGTG, AGTG, GATG, CATG, GGTG, and CCTG. A non-limiting example of a guide that targets a PAM of TCTG has a spacer sequence of SEQ ID NO: 2018. A non-limiting example of a guide that targets a PAM of GGTG has a spacer sequence of SEQ ID NO: 2019. A non-limiting example of a guide that targets a PAM of AGTG has a spacer sequence of SEQ ID NO: 2020. A non-limiting example of a guide that targets a PAM of GATG has a spacer sequence of SEQ ID NO: 2021. A non-limiting example of a guide that targets a PAM of CATG has a spacer sequence of SEQ ID NO: 2022. A non-limiting example of a guide that targets a PAM of TCTA has a spacer sequence of SEQ ID NO: 2023. A non-limiting example of a guide that targets a PAM of GGTG has a spacer sequence of SEQ ID NO: 2024. A non-limiting example of a guide that targets a PAM of CCTG has a spacer sequence of SEQ ID NO: 2025. Another non-limiting example of a guide that targets a PAM of CCTG has a spacer sequence of SEQ ID NO: 2026.

### Nuclease-Dead Effector Proteins

In some embodiments, the effector protein may comprise an enzymatically inactive and/or “dead” (abbreviated by “d”) effector protein in combination (e.g., fusion) with a polypeptide comprising recombinase activity. In some embodiments, nuclease-dead effector protein may also be referred to as a catalytically inactive effector protein. Although an effector protein normally has nuclease activity, in some embodiments, an effector protein does not have nuclease activity. In some embodiments, an effector protein comprising a nuclease-dead effector protein, wherein the nuclease-dead effector protein comprising an amino acid sequence that is at least 60%, at least 65%, at least 70%, at least 75%, at least 80%, at least 85%, at least 90%, at least 92%, at least 95%, at least 97%, at least 98%, at least 99%, or 100% identical to any one of the sequences recited in TABLES 15-19. In some embodiments, the effector protein comprising an amino acid sequence that is at least 60%, at least 65%, at least 70%, at least 75%, at least 80%, at least 85%, at least 90%, at least 92%, at least 95%, at least 97%, at least 98%, at least 99%, or 100% identical to any one of the sequences recited in TABLES 15-19, wherein the effector protein is modified or engineered to be a nuclease-dead effector protein.

Catalytically inactive effector proteins may comprise a modified form of a wildtype counterpart. The modified form of the wildtype counterpart may comprise an amino acid change (e.g., deletion, insertion, or substitution) that reduces the nucleic acid-cleaving activity of the effector protein. In such embodiments, the catalytically inactive effector protein may also be referred to as a catalytically reduced effector protein. For example, a nuclease domain (e.g., HEPN domain, RuvC domain) of an effector protein can be deleted or mutated so that it is no longer functional or comprises reduced nuclease activity. The modified form of the effector protein may have less than 90%, less than 80%, less than 70%, less than 60%, less than 50%, less than 40%, less than 30%, less than 20%, less than 10%, less than 5%, or less than 1% of the nucleic acid-cleaving activity of the wild-type counterpart. The modified form of an effector protein may have no substantial nucleic acid-cleaving activity. When an effector protein is a modified form that has no substantial nucleic acid-cleaving activity, it may be referred to as enzymatically inactive and/or dead. A dead effector polypeptide (e.g., catalytically inactive effector protein) may bind to a target nucleic acid but may not cleave the target nucleic acid. A dead effector polypeptide (e.g., catalytically inactive effector protein) may associate with a guide nucleic acid to activate or repress transcription of a target nucleic acid.

In some embodiments, a nuclease-dead effector protein comprises an amino acid sequence that is at least 60%, at least 65%, at least 70%, at least 75%, at least 80%, at least 85%, at least 90%, at least 92%, at least 95%, at least 97%, at least 98%, or at least 99% identical to SEQ ID NO: 32, and wherein the effector protein further comprises one or more alterations selected from D369A, D369N, E567A, E567Q, D658A and D658N. In some embodiments, a nuclease-dead effector protein comprises an amino acid sequence that is at least 60%, at least 65%, at least 70%, at least 75%, at least 80%, at least 85%, at least 90%, at least 92%, at least 95%, at least 97%, at least 98%, at least 99%, or 100% similar to SEQ ID NO: 32, and wherein the effector protein further comprises one or more alterations selected from D369A, D369N, E567A, E567Q, D658A and D658N.

In certain embodiments, the amino acid sequence of the dCas protein is based on SEQ ID NO: 32 and is modified at position 369. In some embodiments, the modification at position 369 is from aspartic acid to alanine (D369A). In some embodiments, the amino acid sequence of the dCas protein is at least 95%, 96%, 97%, 98%, or 99% identical to SEQ ID NO: 63. In some embodiments, the amino acid sequence of the dCas protein comprises or consists of SEQ ID NO: 63. In certain embodiments, the amino acid sequence of the dCas protein is based on SEQ ID NO: 32 and is modified at position 369. In some embodiments, the modification at position 369 is from aspartic acid to asparagine (D369N). In some embodiments, the amino acid sequence of the dCas protein is at least 95%, 96%, 97%, 98%, or 99% identical to SEQ ID NO: 64. In some embodiments, the amino acid sequence of the dCas protein comprises or consists of SEQ ID NO: 64.

In certain embodiments, the amino acid sequence of the dCas protein is based on SEQ ID NO: 32 and is modified at position 658. In some embodiments, the modification at position 658 is from aspartic acid to alanine (D658A). In some embodiments, the amino acid sequence of the dCas protein is at least 95%, 96%, 97%, 98%, or 99% identical to SEQ ID NO: 65. In some embodiments, the amino acid sequence of the dCas protein comprises or consists of SEQ ID NO: 65.

In certain embodiments, the amino acid sequence of the dCas protein is based on SEQ ID NO: 32 and is modified at position 658. In some embodiments, the modification at position 658 is from aspartic acid to asparagine (D658N). In some embodiments, the amino acid sequence of the dCas protein is at least 95%, 96%, 97%, 98%, or 99% identical to SEQ ID NO: 66. In some embodiments, the amino acid sequence of the dCas protein comprises or consists of SEQ ID NO: 66.

In certain embodiments, the amino acid sequence of the dCas protein is based on SEQ ID NO: 32 and is modified at position 567. In some embodiments, the modification at position 567 is from glutamine acid to alanine (E567A). In some embodiments, the amino acid sequence of the dCas protein is at least 95%, 96%, 97%, 98%, or 99% identical to SEQ ID NO: 45. In some embodiments, the amino acid sequence of the dCas protein comprises or consists of SEQ ID NO: 45.

In certain embodiments, the amino acid sequence of the dCas protein is based on SEQ ID NO: 32 and is modified at position 567. In some embodiments, the modification at position 567 is from glutamic acid to glutamine (E567Q). In some embodiments, the amino acid sequence of the dCas protein is at least 95%, 96%, 97%, 98%, or 99% identical to SEQ ID NO: 46. In some embodiments, the amino acid sequence of the dCas protein comprises or consists of SEQ ID NO: 46.

In some embodiments, a nuclease-dead effector protein comprises an amino acid sequence that is at least 60%, at least 65%, at least 70%, at least 75%, at least 80%, at least 85%, at least 90%, at least 92%, at least 95%, at least 97%, at least 98%, or at least 99% identical to SEQ ID NO: 773, and wherein the effector protein further comprises one or more alterations selected from D237A, D418A, D418N, E335A, and E335Q. In some embodiments, a nuclease-dead effector protein comprises an amino acid sequence that is at least 60%, at least 65%, at least 70%, at least 75%, at least 80%, at least 85%, at least 90%, at least 92%, at least 95%, at least 97%, at least 98%, at least 99%, or 100% similar to SEQ ID NO: 773, and wherein the effector protein further comprises one or more alterations selected from D237A, D418A, D418N, E335A, and E335Q.

In certain embodiments, the amino acid sequence of the dCas protein is based on SEQ ID NO: 773 and is modified at position 335. In some embodiments, the modification at position 335 is from glutamic acid to glutamine (E335Q). In some embodiments, the amino acid sequence of the dCas protein is at least 95%, 96%, 97%, 98%, or 99% identical to SEQ ID NO: 788. In some embodiments, the amino acid sequence of the dCas protein comprises or consists of SEQ ID NO: 788.

In certain embodiments, the amino acid sequence of the dCas protein is based on SEQ ID NO: 773 and is modified at position 237. In some embodiments, the modification at position 237 is from aspartic acid to alanine (D237A). In some embodiments, the amino acid sequence of the dCas protein is at least 95%, 96%, 97%, 98%, or 99% identical to SEQ ID NO: 789. In some embodiments, the amino acid sequence of the dCas protein comprises or consists of SEQ ID NO: 789.

In certain embodiments, the amino acid sequence of the dCas protein is based on SEQ ID NO: 773 and is modified at position 418. In some embodiments, the modification at position 418 is from aspartic acid to alanine (D418A). In some embodiments, the amino acid sequence of the dCas protein is at least 95%, 96%, 97%, 98%, or 99% identical to SEQ ID NO: 790. In some embodiments, the amino acid sequence of the dCas protein comprises or consists of SEQ ID NO: 790.

In certain embodiments, the amino acid sequence of the dCas protein is based on SEQ ID NO: 773 and is modified at position 418. In some embodiments, the modification at position 418 is from aspartic acid to asparagine (D418N). In some embodiments, the amino acid sequence of the dCas protein is at least 95%, 96%, 97%, 98%, or 99% identical to SEQ ID NO: 791. In some embodiments, the amino acid sequence of the dCas protein comprises or consists of SEQ ID NO: 791.

In certain embodiments, the amino acid sequence of the dCas protein is based on SEQ ID NO: 773 and is modified at position 335. In some embodiments, the modification at position 335 is from glutamic acid to alanine (E335A). In some embodiments, the amino acid sequence of the dCas protein is at least 95%, 96%, 97%, 98%, or 99% identical to SEQ ID NO: 792. In some embodiments, the amino acid sequence of the dCas protein comprises or consists of SEQ ID NO: 792.

### Fusion Proteins

In some embodiments, compositions, systems, and methods comprise a fusion protein, a fusion partner, or uses thereof. A fusion protein generally comprises an effector protein and a fusion partner. In some embodiments, the fusion partner comprises a polypeptide or peptide that is linked to the effector protein. In some embodiments, the fusion partner is not linked to the effector protein but is brought into proximity of the effector protein by other means. By way of non-limiting example, a fusion partner protein may comprise a peptide that binds an aptamer of a guide nucleic acid, wherein the effector protein is also capable of binding the guide nucleic acid, the guide nucleic acid thereby bringing the fusion partner into proximity of the effector protein. In some embodiments, the fusion partner is capable of binding or being bound by an effector protein. In some embodiments, the fusion partner and the effector protein are both capable of binding or being bound by an additional protein or moiety, the additional protein or moiety thereby bringing the fusion partner into proximity of the effector protein. In some embodiments, the fusion protein is a heterologous peptide or polypeptide as described herein. In some embodiments, the amino terminus of the fusion partner is linked to the carboxy terminus of the effector protein. In some embodiments, the carboxy terminus of the fusion partner protein is linked to the amino terminus of the effector protein by the linker. In some embodiments, the fusion partner is not an effector protein as described herein. In some embodiments, the fusion partner comprises a second effector protein or a multimeric form thereof. Accordingly, in some embodiments, the fusion protein comprises more than one effector protein. In such embodiments, the fusion protein can comprise at least two effector proteins that are same. In some embodiments, the fusion protein comprises at least two effector proteins that are different. In some embodiments, the multimeric form is a homomeric form. In some embodiments, the multimeric form is a heteromeric form. Unless otherwise indicated, reference to effector proteins throughout the present disclosure include fusion proteins comprising the effector protein described herein and a fusion partner.

In some embodiments, a fusion partner imparts some function or activity to a fusion protein that is not provided by an effector protein. Such activities may include but are not limited to nuclease activity, methyltransferase activity, demethylase activity, DNA repair activity, DNA damage activity, deamination activity, dismutase activity, alkylation activity, depurination activity, oxidation activity, dimer forming activity (e.g., pyrimidine dimer forming activity), integrase activity, transposase activity, recombinase activity, polymerase activity, ligase activity, helicase activity, photolyase activity, glycosylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity or demyristoylation activity, modification of a polypeptide associated with target nucleic acid (e.g., a histone), and/or signaling activity.

In some embodiments, a fusion partner may provide signaling activity. In some embodiments, a fusion partner may inhibit or promote the formation of multimeric complex of an effector protein. In an additional example, the fusion partner may directly or indirectly edit a target nucleic acid. Edits can be of a nucleobase, nucleotide, or nucleotide sequence of a target nucleic acid. In some embodiments, the fusion partner may interact with additional proteins, or functional fragments thereof, to make modifications to a target nucleic acid. In other embodiments, the fusion partner may modify proteins associated with a target nucleic acid. In some embodiments, a fusion partner may modulate transcription (e.g., inhibits transcription, increases transcription) of a target nucleic acid. In yet another example, a fusion partner may directly or indirectly inhibit, reduce, activate or increase expression of a target nucleic acid.

In some embodiments of the above, the systems and compositions provided herein comprise a fusion protein comprising an effector protein comprising an amino acid sequence that is at least 90% identical to any one of the sequences recited in TABLES 15, 18, and 19, and a guide RNA comprising a repeat sequence that is at least 90% identical to any one of SEQ ID NOs: 16 or 38-43 and a spacer sequence that is at least 90% identical to any one of the sequences recited in TABLES 1, 3, and 5.

In some embodiments of the above, the systems and compositions provided herein comprise a fusion protein comprising an effector protein comprising an amino acid sequence this is at least 95% identical to any one of the sequences recited in TABLES 15, 18, and 19, and wherein a guide RNA comprising a repeat sequence that is at least 95% to any one of SEQ ID NOs: 16 or 38-43 and a spacer sequence that is at least 95% identical to any one of the sequences recited in TABLES 1, 3, and 5.

In some embodiments of the above, the systems and compositions provided herein comprise a fusion protein comprising an effector protein comprising any one of the sequences recited in TABLES 15, 18, and 19, and a guide RNA comprising any one of SEQ ID NOs: 16 or 38-43 and any one of the spacer sequences recited in TABLES 1, 3, and 5.

In some embodiments, the effector protein comprises an amino acid sequence this is at least 90% identical to any one of the sequences of TABLES 15, 18, and 19, and wherein the guide RNA comprises a sequence that is at least 90% identical to any one of the guide RNA sequences of TABLES 8-10.

In some embodiments of the above, the systems and compositions provided herein comprise a fusion protein comprising an effector protein comprising an amino acid sequence that is at least 95% identical to any one of the sequences of TABLES 15, 18, and 19, and a guide RNA comprising a sequence that is at least 95% identical to any one of the guide RNA sequences of TABLES 8-10.

In some embodiments of the above, the effector protein comprises any one of the sequences recited in TABLES 15, 18, and 19, and wherein the guide RNA comprises a sequence recited in TABLES 8-10.

In some embodiments of the above, the systems and compositions provided herein comprise a fusion protein comprising an effector protein comprising an amino acid sequence that is at least 90% identical to any one of the sequences recited in TABLES 15-17, and a guide RNA comprising a repeat sequence that is at least 90% identical to SEQ ID NO: 488 and a spacer sequence that is at least 90% identical to any one of the sequences recited in TABLES 2, 4, and 6.

In some embodiments of the above, the systems and compositions provided herein comprise a fusion protein comprising an effector protein comprising an amino acid sequence this is at least 95% identical to any one of the sequences recited in TABLES 15-17, and a guide RNA comprising a repeat sequence that is at least 95% identical to SEQ ID NO: 488 and a spacer sequence that is at least 95% identical to any one of the sequences recited in TABLES 2, 4, and 6.

In some embodiments of the above, the effector protein comprises any one of the sequences recited in TABLES 15-17, and wherein the guide RNA comprises a repeat that is identical to SEQ ID NO: 488 and any one of the spacer sequences recited in TABLES 2, 4, and 6.

In some embodiments of the above, the systems and compositions provided herein comprise a fusion protein comprising an effector protein comprising an amino acid sequence this is at least 90% identical to any one of the sequences of TABLES 15-17, and a guide RNA comprising a sequence that is at least 90% identical to any one of the guide RNA sequences of TABLEs 11-13.

In some embodiments of the above, the systems and compositions provided herein comprise a fusion protein comprising an effector protein comprising an amino acid sequence that is at least 95% identical to any one of the sequences of TABLES 15-17, and a guide RNA comprising a sequence that is at least 95% identical to any one of the guide RNA sequences of TABLEs 11-13.

In some embodiments of the above, the systems and compositions provided herein comprise a fusion protein comprising an effector protein comprising any one of the sequences recited in TABLES 15-17, and a guide RNA comprising a sequence recited in TABLE 11-13.

In some embodiments of the above, the systems and compositions provided herein comprise a fusion protein comprising an guide RNA comprising at least one sequence that is at least 80%, at least 85%, at least 90%, at least 95% or 100% identical to a sequence selected from any one of TABLES 1, 7, and 8.

In some embodiments of the above, the systems and compositions provided herein comprise a fusion protein comprising an guide RNA comprising at least one sequence that is at least 80%, at least 85%, at least 90%, at least 95% or 100% identical to a sequence selected from any one of TABLES 3, 7, and 9.

In some embodiments of the above, the systems and compositions provided herein comprise a fusion protein comprising an guide RNA comprising at least one sequence that is at least 80%, at least 85%, at least 90%, at least 95% or 100% identical to a sequence selected from any one of TABLES 5, 7, and 10.

In some embodiments of the above, the guide RNA comprises at least one sequence that is at least 80%, at least 85%, at least 90%, at least 95% or 100% identical to a sequence selected from any one of TABLES 2, 7, and 11.

In some embodiments of the above, the systems and compositions provided herein comprise a fusion protein comprising an guide RNA comprising at least one sequence that is at least 80%, at least 85%, at least 90%, at least 95% or 100% identical to a sequence selected from any one of TABLES 4, 7, and 12.

In some embodiments of the above, the systems and compositions provided herein comprise a fusion protein comprising an guide RNA comprising at least one sequence that is at least 80%, at least 85%, at least 90%, at least 95% or 100% identical to a sequence selected from any one of TABLES 6, 7, and 13.

In some embodiments, of the above, the systems and compositions provided herein comprise a fusion protein comprising an effector protein amino acid sequence comprises a nuclear localization signal.

In some embodiments, of the above, the systems and compositions provided herein comprise a fusion protein comprising an composition further comprises an additional guide RNA that binds a different portion of the target nucleic acid than the guide RNA.

### Nucleic Acid Modification Activity

In some embodiments, fusion partners have enzymatic activity that modifies a nucleic acid, such as a target nucleic acid. In some embodiments, the target nucleic acid may comprise or consist of a ssRNA, dsRNA, ssDNA, or a dsDNA. Examples of enzymatic activity that modifies the target nucleic acid include, but are not limited to: nuclease activity, which comprises the enzymatic activity of an enzyme which allows the enzyme to cleave the phosphodiester bonds between the nucleotide subunits of nucleic acids, such as that provided by a restriction enzyme, or a nuclease (e.g., FokI nuclease); methyltransferase activity such as that provided by a methyltransferase (e.g., HhaI DNA m5c-methyltransferase (M.HhaI), DNA methyltransferase 1 (DNMT1), DNA methyltransferase 3a (DNMT3a), DNA methyltransferase 3b (DNMT3b), METI, DRM3 (plants), ZMET2, CMT1, CMT2 (plants)); demethylase activity such as that provided by a demethylase (e.g., Ten-Eleven Translocation (TET) dioxygenase 1 (TET1CD), TET1, DME, DML1, DML2, ROS1); DNA repair activity; DNA damage (e.g., oxygenation) activity; deamination activity such as that provided by a deaminase (e.g., a cytosine deaminase enzyme such as rat APOBEC1); dismutase activity; alkylation activity; depurination activity; oxidation activity; pyrimidine dimer forming activity; integrase activity such as that provided by an integrase and/or resolvase (e.g., Gin invertase such as the hyperactive mutant of the Gin invertase, GinH106Y, human immunodeficiency virus type 1 integrase (IN), Tn3 resolvase); transposase activity; recombinase activity such as that provided by a recombinase (e.g., catalytic domain of Gin recombinase); polymerase activity; ligase activity; helicase activity; photolyase activity; and glycosylase activity.

In some embodiments, fusion partners target a ssRNA, dsRNA, ssDNA, or a dsDNA. In some embodiments, fusion partners target ssRNA. Non-limiting examples of fusion partners for targeting ssRNA include, but are not limited to, splicing factors (e.g., RS domains); protein translation components (e.g., translation initiation, elongation, and/or release factors; e.g., eIF4G); RNA methylases; RNA editing enzymes (e.g., RNA deaminases, e.g., adenosine deaminase acting on RNA (ADAR), including A to I and/or C to U editing enzymes); helicases; and RNA-binding proteins.

It is understood that a fusion partner may include an entire protein, or in some embodiments, may include a fragment of the protein (e.g., a functional domain). In some embodiments, the functional domain binds or interacts with a nucleic acid, such as ssRNA, including intramolecular and/or intermolecular secondary structures thereof (e.g., hairpins, stem-loops, etc.). The functional domain may interact transiently or irreversibly, directly, or indirectly. In some embodiments, a functional domain comprises a region of one or more amino acids in a protein that is required for an activity of the protein, or the full extent of that activity, as measured in an in vitro assay. Activities include but are not limited to nucleic acid binding, nucleic acid editing, nucleic acid mutating, nucleic acid modifying, nucleic acid cleaving, protein binding or combinations thereof. The absence of the functional domain, including mutations of the functional domain, would abolish or reduce activity.

Accordingly, fusion partners may comprise a protein or domain thereof selected from: endonucleases (e.g., RNase III, the CRR22 DYW domain, Dicer, and PIN (PilT N-terminus); SMG5 and SMG6; domains responsible for stimulating RNA cleavage (e.g., CPSF, CstF, CFIm and CFIIm); exonucleases such as XRN-1 or Exonuclease T; deadenylases such as HNT3; protein domains responsible for nonsense mediated RNA decay (e.g., UPF1, UPF2, UPF3, UPF3b, RNP S1, Y14, DEK, REF2, and SRml60); protein domains responsible for stabilizing RNA (e.g., PABP); proteins and protein domains responsible for polyadenylation of RNA (e.g., PAP1, GLD-2, and Star-PAP); proteins and protein domains responsible for polyuridinylation of RNA (e.g., CID1 and terminal uridylate transferase); and other suitable domains that affect nucleic acid modifications.

In some embodiments, an effector protein is a fusion protein, wherein the effector protein is linked to a chromatin-modifying enzyme. In some embodiments, the fusion protein chemically modifies a target nucleic acid, for example by methylating, demethylating, or acetylating the target nucleic acid in a sequence specific or non-specific manner.

### Base Editors

In some embodiments, fusion partners edit a nucleobase of a target nucleic acid. Fusion proteins comprising such a fusion partner and an effector protein may be referred to as base editors. Such a fusion partner may be referred to as a base editing enzyme. In some embodiments, a base editor comprises a base editing enzyme variant that differs from a naturally occurring base editing enzyme, but it is understood that any reference to a base editing enzyme herein also refers to a base editing enzyme variant. In some embodiments, a base editor may be a fusion protein comprising a base editing enzyme linked to an effector protein. In some embodiments, the amino terminus of the fusion partner protein is linked to the carboxy terminus of the effector protein by the linker. In some embodiments, the carboxy terminus of the fusion partner protein is linked to the amino terminus of the effector protein by the linker. The base editor may be functional when the effector protein is coupled to a guide nucleic acid. The base editor may be functional when the effector protein is coupled to a guide nucleic acid. The guide nucleic acid imparts sequence specific activity to the base editor. By way of non-limiting example, the effector protein may comprise a catalytically inactive effector protein (e.g., a catalytically inactive variant of an effector protein described herein). Also, by way of non-limiting example, the base editing enzyme may comprise deaminase activity. Additional base editors are described herein.

In some embodiments, base editors are capable of catalyzing editing (e.g., a chemical modification) of a nucleobase of a nucleic acid molecule, such as DNA or RNA (single stranded or double stranded). In some embodiments, a base editing enzyme, and therefore a base editor, is capable of converting an existing nucleobase to a different nucleobase, such as: an adenine (A) to guanine (G); cytosine (C) to thymine (T); cytosine (C) to guanine (G); uracil (U) to cytosine (C); guanine (G) to adenine (A); hydrolytic deamination of an adenine or adenosine, or methylation of cytosine (e.g., CpG, CpA, CpT or CpC). In some embodiments, base editors edit a nucleobase on a ssDNA. In some embodiments, base editors edit a nucleobase on both strands of dsDNA. In some embodiments, base editors edit a nucleobase of an RNA.

In some embodiments, a base editing enzyme itself may or may not bind to the nucleic acid molecule containing the nucleobase. In some embodiments, upon binding to its target locus in the target nucleic acid (e.g., a DNA molecule), base pairing between the guide nucleic acid and target strand leads to displacement of a small segment of ssDNA in an “R-loop”. In some embodiments, DNA bases within the R-loop are edited by the base editor having the deaminase enzyme activity. In some embodiments, base editors for improved efficiency in eukaryotic cells comprise a catalytically inactive effector protein that may generate a nick in the non-edited strand, inducing repair of the non-edited strand using the edited strand as a template.

In some embodiments, a base editing enzyme comprises a deaminase enzyme. Exemplary deaminases are described in US20210198330, WO2021041945, WO2021050571A1, and WO2020123887, all of which are incorporated herein by reference in their entirety. Exemplary deaminase domains are described WO2018027078 and WO2017070632, and each are hereby incorporated in its entirety by reference. Also, additional exemplary deaminase domains are described in Komor et al., Nature, 533, 420-424 (2016); Gaudelli et al., Nature, 551, 464-471 (2017); Komor et al., Science Advances, 3:eaao4774 (2017), and Rees et al., Nat Rev Genet. 2018 December; 19(12):770-788. doi: 10.1038/s41576-018-0059-1, which are hereby incorporated by reference in their entirety. In some embodiments, the deaminase functions as a monomer. In some embodiments, the deaminase functions as heterodimer with an additional protein. In some embodiments, base editors comprise a DNA glycosylase inhibitor (e.g., an uracil glycosylase inhibitor (UGI) or uracil N-glycosylase (UNG)). In some embodiments, the fusion partner is a deaminase, e.g., ADAR1/2, ADAR-2, AID, or any function variant thereof.

In some embodiments, a base editor is a cytosine base editor (CBE). In some embodiments, the CBE may convert a cytosine to a thymine. In some embodiments, a cytosine base editing enzyme may accept ssDNA as a substrate but may not be capable of cleaving dsDNA, as it is linked to a catalytically inactive effector protein. In some embodiments, when bound to its cognate DNA, the catalytically inactive effector protein of the CBE may perform local denaturation of the DNA duplex to generate an R-loop in which the DNA strand not paired with a guide nucleic acid exists as a disordered single-stranded bubble. In some embodiments, the catalytically inactive effector protein generated ssDNA R-loop may enable the CBE to perform efficient and localized cytosine deamination in vitro. In some embodiments, deamination activity is exhibited in a window of about 4 to about 10 base pairs. In some embodiments, fusion to the catalytically inactive effector protein presents a target site to the cytosine base editing enzyme in high effective molarity, which may enable the CBE to deaminate cytosines located in a variety of different sequence motifs, with differing efficacies. In some embodiments, the CBE is capable of mediating RNA-programmed deamination of target cytosines in vitro or in vivo. In some embodiments, the cytosine base editing enzyme is a cytidine deaminase. In some embodiments, the cytosine base editing enzyme is a cytosine base editing enzyme described by Koblan et al. (2018) Nature Biotechnology 36:848-846; Komor et al. (2016) Nature 533:420-424; Koblan et al. (2021) “Efficient C⋅G-to-G⋅C base editors developed using CRISPRi screens, target-library analysis, and machine learning,” Nature Biotechnology; Kurt et al. (2021) Nature Biotechnology 39:41-46; Zhao et al. (2021) Nature Biotechnology 39:35-40; and Chen et al. (2021) Nature Communications 12:1384, all incorporated herein by reference.

In some embodiments, CBEs comprise a uracil glycosylase inhibitor (UGI) or uracil N-glycosylase (UNG). In some embodiments, base excision repair (BER) of U⋅G in DNA is initiated by a UNG, which recognizes a U⋅G mismatch and cleaves the glyosidic bond between a uracil and a deoxyribose backbone of DNA. In some embodiments, BER results in the reversion of the U⋅G intermediate created by the first CBE back to a C⋅G base pair. In some embodiments, the UNG may be inhibited by fusion of a UGI. In some embodiments, the CBE comprises a UGI. In some embodiments, a C-terminus of the CBE comprises the UGI. In some embodiments, the UGI is a small protein from bacteriophage PBS. In some embodiments, the UGI is a DNA mimic that potently inhibits both human and bacterial UNG. In some embodiments, the UGI inhibitor is any protein or polypeptide that inhibits UNG. In some embodiments, the CBE may mediate efficient base editing in bacterial cells and moderately efficient editing in mammalian cells, enabling conversion of a C⋅G base pair to a T⋅A base pair through a U⋅G intermediate. In some embodiments, the CBE is modified to increase base editing efficiency while editing more than one strand of DNA.

In some embodiments, a CBE nicks a non-edited DNA strand. In some embodiments, the non-edited DNA strand nicked by the CBE biases cellular repair of a U⋅G mismatch to favor a U⋅A outcome, elevating base editing efficiency. In some embodiments, a APOBEC1-nickase-UGI fusion efficiently edits in mammalian cells, while minimizing frequency of non-target indels. In some embodiments, base editors do not comprise a functional fragment of the base editing enzyme. In some embodiments, base editors do not comprise a function fragment of a UGI, where such a fragment may be capable of excising a uracil residue from DNA by cleaving an N-glycosidic bond.

In some embodiments, the fusion protein further comprises a non-protein uracil-DNA glycosylase inhibitor (npUGI). In some embodiments, the npUGI is selected from a group of small molecule inhibitors of uracil-DNA glycosylase (UDG), or a nucleic acid inhibitor of UDG. In some embodiments, the npUGI is a small molecule derived from uracil. Examples of small molecule non-protein uracil-DNA glycosylase inhibitors, fusion proteins, and Cas-CRISPR systems comprising base editing activity are described in WO2021087246, which is incorporated by reference in its entirety.

In some embodiments, a cytosine base editing enzyme, and therefore a cytosine base editor, is a cytidine deaminase. In some embodiments, the cytidine deaminase base editor is generated by ancestral sequence reconstruction as described in WO2019226953, which is hereby incorporated by reference in its entirety. Non-limiting exemplary cytidine deaminases suitable for use with effector proteins described herein include: APOBEC1, APOBEC2, APOBEC3C, APOBEC3D, APOBEC3F, APOBEC3G, APOBEC3H, APOBEC4, APOBEC3A, BE1 (APOBEC1-XTEN-dCas9), BE2 (APOBEC1-XTEN-dCas9-UGI), BE3 (APOBEC1-XTEN-dCas9(A840H)-UGI), BE3-Gam, saBE3, saBE4-Gam, BE4, BE4-Gam, saBE4, and saBE4-Gam as described in WO2021163587, WO2021087246, WO2021062227, and WO2020123887, which are incorporated herein by reference in their entirety.

In some embodiments, a base editor is a cytosine to guanine base editor (CGBE). A CGBE may convert a cytosine to a guanine.

In some embodiments, a base editor is an adenine base editor (ABE). An ABE may convert an adenine to a guanine. In some embodiments, an ABE converts an A⋅T base pair to a G⋅C base pair. In some embodiments, the ABE converts a target A⋅T base pair to G⋅C in vivo or in vitro. In some embodiments, ABEs provided herein reverse spontaneous cytosine deamination, which has been linked to pathogenic point mutations. In some embodiments, ABEs provided herein enable correction of pathogenic SNPs (˜47% of disease-associated point mutations). In some embodiments, the adenine comprises exocyclic amine that has been deaminated (e.g., resulting in altering its base pairing preferences). In some embodiments, deamination of adenosine yields inosine. In some embodiments, inosine exhibits the base-pairing preference of guanine in the context of a polymerase active site, although inosine in the third position of a tRNA anticodon is capable of pairing with A, U, or C in mRNA during translation. Non-limiting exemplary adenine base editing enzymes suitable for use with effector proteins described herein include: ABE8e, ABE8.20m, APOBEC3A, Anc APOBEC (a.k.a. AncBE4Max), and BtAPOBEC2. Non-limiting exemplary ABEs suitable for use herein include: ABE7, ABE8.1m, ABE8.2m, ABE8.3m, ABE8.4m, ABE8.5m, ABE8.6m, ABE8.7m, ABE8.8m, ABE8.9m, ABE8.10m, ABE8.11m, ABE8.12m, ABE8.13m, ABE8.14m, ABE8.15m, ABE8.16m, ABE8.17m, ABE8.18m, ABE8.19m, ABE8.20m, ABE8.21m, ABE8.22m, ABE8.23m, ABE8.24m, ABE8.1d, ABE8.2d, ABE8.3d, ABE8.4d, ABE8.5d, ABE8.6d, ABE8.7d, ABE8.8d, ABE8.9d, ABE8.10d, ABE8.11d, ABE8.12d, ABE8.13d, ABE8.14d, ABE8.15d, ABE8.16d, ABE8.17d, ABE8.18d, ABE8.19d, ABE8.20d, ABE8.21d, ABE8.22d, ABE8.23d, and ABE8.24d. In some embodiments, the adenine base editing enzyme is an adenine base editing enzyme described in Chu et al., (2021) The CRISPR Journal 4:2:169-177, incorporated herein by reference. In some embodiments, the adenine deaminase is an adenine deaminase described by Koblan et al. (2018) Nature Biotechnology 36:848-846, incorporated herein by reference. In some embodiments, the adenine base editing enzyme is an adenine base editing enzyme described by Tran et al. (2020) Nature Communications 11:4871.

In some embodiments, the ABE is ABE8e and comprises an amino acid sequence that is at least at least 90%, at least 95%, at least 97%, at least 98%, or at least 99% identical to SEQ ID NO: 796. In some embodiments, the ABE is ABE8e and comprises or consists of SEQ ID NO: 796.

In some embodiments, the present disclosure provides a fusion protein comprising an effector protein described herein and a base editing enzyme described herein. In some embodiments, the fusion protein comprises, from N-terminus to C-terminus, an effector protein and a base editing enzyme. In some embodiments, the fusion protein comprises, from N-terminus to C-terminus, a base editing enzyme and an effector protein. In some embodiments, the base editing enzyme is ABE8e.

In some embodiments, the fusion protein described herein comprises an effector protein comprising an amino acid sequence that is at least 90%, 91%, 92%, 93%, 94%, 95%, 96%, 97%, 98%, or 99% identical to SEQ ID NO: 773 and a base editing enzyme comprising an amino acid sequence that is at least 90%, 91%, 92%, 93%, 94%, 95%, 96%, 97%, 98%, or 99% identical to SEQ ID NO: 796. In some embodiments, the fusion protein described herein comprises an effector protein comprising or consisting of SEQ ID NO: 773 and a base editing enzyme comprising or consisting of SEQ ID NO: 796. In some embodiments, the fusion protein comprises a linker sequence comprising SEQ ID NO: 795. In some embodiments, the fusion protein comprises an amino acid sequence that is at least at least 90%, at least 95%, at least 97%, at least 98%, or at least 99% identical to SEQ ID NO: 797. In some embodiments, the ABE is ABE8e and comprises or consists of SEQ ID NO: 797.

In some embodiments, the fusion protein described herein comprises an effector protein comprising an amino acid sequence that is at least 90%, 91%, 92%, 93%, 94%, 95%, 96%, 97%, 98%, or 99% identical to SEQ ID NO: 32 and a base editing enzyme comprising an amino acid sequence that is at least 90%, 91%, 92%, 93%, 94%, 95%, 96%, 97%, 98%, or 99% identical to SEQ ID NO: 796. In some embodiments, the fusion protein described herein comprises an effector protein comprising or consisting of SEQ ID NO: 32 and a base editing enzyme comprising or consisting of SEQ ID NO: 796. In some embodiments, the fusion protein comprises a linker sequence comprising SEQ ID NO: 795. In some embodiments, the fusion protein comprises an amino acid sequence that is at least at least 90%, at least 95%, at least 97%, at least 98%, or at least 99% identical to SEQ ID NO: 798. In some embodiments, the ABE is ABE8e and comprises or consists of SEQ ID NO: 798. Exemplary fusion proteins are provided in TABLE 23.

TABLE 23
Exemplary base editing enzyme and
base editor fusion proteins

| | | SEQ |
|---|---|---|
| Protein | AA Sequence | ID |
| ABE8e | SEVEFSHEYWMRHALTLAKRARDEREVPVG | 796 |
| | AVLVLNNRVIGEGWNRAIGLHDPTAHAEIM | |
| | ALRQGGLVMQNYRLIDATLYVTFEPCVMCA | |
| | GAMIHSRIGRVVFGVRNSKRGAAGSLMNVL | |
| | NYPGMNHRVEITEGILADECAALLCDFYRM | |
| | PRQVFNAQKKAQSSIN | |
| CasM. | MSVLTRKVQLIPVGDKEERDRVYKYLRDGI | |
| 265466- | EAQNRAMNLYMSGLYFAAINEASKEDRKEL | 797 |
| D220R- | NQLYSRIATSSKGSAYTTDIEFPTGLASTS | |
| E335Q_ABE8e | TLSMAVRQDFTKSLKDGLMYGRVSLPTYRK | |
| fusion | DNPLFVDVRFVALRGTKQKYNGLYHEYKSH | |
| | TEFLDNLYSSDLKVYIKFANDITFQVIFGN | |
| | PRKSSALRSEFQNIFEEYYKVCQSSIQFSG | |
| | TKIILNMAMRIPDKEIELDEDVCVGVDLGI | |
| | AIPAVCALNKNRYSRVSIGSKEDFLRVRTK | |
| | IRNQRKRLQTNLKSSNGGHGRKKKMKPMDR | |
| | FRDYEANWVQNYNHYVSRQVVDFAVKNKAK | |
| | YINLQNLEGIRDDVKNEWLLSNWSYYQLQQ | |
| | YITYKAKTYGIEVRKINPYHTSQRCSCCGY | |
| | EDAGNRPKKEKGQAYFKCLKCGEEMNADFN | |
| | AARNIAMSTEFQSGKKTKKQKKEQHENKGS | |
| | SGGSPAGSPTSTEEGTSESATPESGPGTST | |
| | EPSEGSAPGSPAGSGGGSSEVEFSHEYWMR | |
| | HALTLAKRARDEREVPVGAVLVLNNRVIGE | |
| | GWNRAIGLHDPTAHAEIMALRQGGLVMQNY | |
| | RLIDATLYVTFEPCVMCAGAMIHSRIGRVV | |
| | FGVRNSKRGAAGSLMNVLNYPGMNHRVEIT | |
| | EGILADECAALLCDFYRMPRQVFNAQKKAQ | |
| | SSIN | |
| CasPhi.12- | MIKPTVSQFLTPGFKLIRNHSRTAGKKLKN | 798 |
| L26K- | EGEEACKKFVRENEIPKDECPNFQGGPAIA | |
| E567Q_ABE8e | NIIAKSREFTEWEIYQSSLAIQEVIFTLPK | |
| fusion | DKLPEPILKEEWRAQWLSEHGLDTVPYKEA | |
| | AGLNLIIKNAVNTYKGVQVKVDNKNKNNLA | |
| | KINRKNEIAKLNGEQEISFEEIKAFDDKGY | |
| | LLQKPSPNKSIYCYQSVSPKPFITSKYHNV | |
| | NLPEEYIGYYRKSNEPIVSPYQFDRLRIPI | |
| | GEPGYVPKWQYTFLSKKENKRRKLSKRIKN | |
| | VSPILGIICIKKDWCVFDMRGLLRTNHWKK | |
| | YHKPTDSINDLFDYFTGDPVIDTKANVVRF | |
| | RYKMENGIVNYKPVREKKGKELLENICDQN | |
| | GSCKLATVDVGQNNPVAIGLFELKKVNGEL | |
| | TKTLISRHPTPIDFCNKITAYRERYDKLES | |
| | SIKLDAIKQLTSEQKIEVDNYNNNFTPQNT | |
| | KQIVCSKLNINPNDLPWDKMISGTHFISEK | |
| | AQVSNKSEIYFTSTDKGKTKDVMKSDYKWF | |
| | QDYKPKLSKEVRDALSDIEWRLRRESLEFN | |
| | KLSKSREQDARQLANWISSMCDVIGIQNLV | |
| | KKNNFFGGSGKREPGWDNFYKPKKENRWWI | |
| | NAIHKALTELSQNKGKRVILLPAMRTSITC | |
| | PKCKYCDSKNRNGEKFNCLKCGIELNADID | |
| | VATENLATVAITAQSMPKPTCERSGDAKKP | |
| | VRARKAKAPEFHDKLAPSYTVVLREAVGSS | |
| | GGSPAGSPTSTEEGTSESATPESGPGTSTE | |
| | PSEGSAPGSPAGSGGGSSEVEFSHEYWMRH | |
| | ALTLAKRARDEREVPVGAVLVLNNRVIGEG | |
| | WNRAIGLHDPTAHAEIMALRQGGLVMQNYR | |
| | LIDATLYVTFEPCVMCAGAMIHSRIGRVVF | |
| | GVRNSKRGAAGSLMNVLNYPGMNHRVEITE | |
| | GILADECAALLCDFYRMPRQVFNAQKKAQS | |
| | SIN | |

In some embodiments, an adenine base editing enzyme of an ABE is an adenosine deaminase. Non-limiting exemplary adenosine base editing enzymes suitable for use herein include ABE9. In some embodiments, the ABE comprises an engineered adenosine deaminase enzyme capable of acting on ssDNA. The engineered adenosine deaminase enzyme may be an adenosine deaminase variant that differs from a naturally occurring deaminase. Relative to the naturally occurring deaminase, the adenosine deaminase variant may comprise one or more amino acid alteration, including a V82S alteration, a T166R alteration, a Y147T alteration, a Y147R alteration, a Q154S alteration, a Y123H alteration, a Q154R alteration, or a combination thereof.

In some embodiments, a base editor comprises a deaminase dimer. In some embodiments, the base editor further comprising a base editing enzyme and an adenine deaminase (e.g., TadA). In some embodiments, the adenosine deaminase is a TadA monomer (e.g., Tad*7.10, TadA*8 or TadA*9). In some embodiments, the adenosine deaminase is a TadA*8 variant (e.g., any one of TadA*8.1, TadA*8.2, TadA*8.3, TadA*8.4, TadA*8.5, TadA*8.6, TadA*8.7, TadA*8.8, TadA*8.9, TadA*8.10, TadA*8.11, TadA*8.12, TadA*8.13, TadA*8.14, TadA*8.15, TadA*8.16, TadA*8.17, TadA*8.18, TadA*8.19, TadA*8.20, TadA*8.21, TadA*8.22, TadA*8.23, or TadA*8.24 as described in WO2021163587 and WO2021050571, which are each hereby incorporated by reference in its entirety). In some embodiments, the base editor comprises a base editing enzyme linked to TadA by a linker (e.g., wherein the base editing enzyme is linked to TadA at N-terminus or C-terminus by a linker).

In some embodiments, a base editing enzyme is a deaminase dimer comprising an ABE. In some embodiments, the deaminase dimer comprises an adenosine deaminase. In some embodiments, the deaminase dimer comprises TadA linked to a suitable adenine base editing enzyme including an: ABE8e, ABE8.20m, APOBEC3A, Anc APOBEC (a.k.a. AncBE4Max), BtAPOBEC2, and variants thereof. In some embodiments, the adenine base editing enzyme is linked to amino-terminus or the carboxy-terminus of TadA.

In some embodiments, RNA base editors comprise an adenosine deaminase. In some embodiments, ADAR proteins bind to RNAs and alter their sequence by changing an adenosine into an inosine. In some embodiments, RNA base editors comprise an effector protein that is activated by or binds RNA.

In some embodiments, base editors are used to treat a subject having or a subject suspected of having a disease related to a gene of interest. In some embodiments, base editors are useful for treating a disease or a disorder caused by a point mutation in a gene of interest. In some embodiments, compositions, systems, and methods described herein comprise a base editor and a guide nucleic acid, wherein the guide nucleic acid directs the base editor to a sequence in a target gene.

### Precision Editing Systems

In some embodiments, the fusion partner comprises a polymerase. In some embodiments, the fusion partner is an RNA-directed DNA polymerase (RDDP). In some embodiments, the RDDP is a reverse transcriptase.

In some embodiments, the RDDP that is capable of catalyzing the modification of the target nucleic acid forms a complex with an extended guide RNA. In some embodiments, the extended guide RNA comprises (not necessarily in this order): a first region (also referred to as a protein binding region or protein binding sequence) that interacts with an effector protein; a second region comprising a spacer sequence that is complementary to a target sequence of a first strand of a target dsDNA molecule; a third region comprising a template sequence that is complementary to at least a portion of the target sequence on the non-target strand of the target dsDNA molecule with the exception of at least one nucleotide; and a fourth region comprising a primer binding sequence that hybridizes to a primer sequence of the target dsDNA molecule that is formed when target nucleic acid is cleaved. The third region or template sequence may comprise a nucleotide having a different nucleobase than that of a nucleotide at the corresponding position in the target nucleic acid when the template sequence and the target sequence are aligned for maximum identity. In some embodiments, there is a linker between any one of the first, second, third and fourth regions. In some embodiments, the linker comprises a nucleotide. In some embodiments, the linker comprises multiple nucleotides.

In some embodiments, the third and fourth regions are 5′ of the first and second regions. In some embodiments, the order of the regions of the extended guide RNA from 5′ to 3′ is: third region, fourth region, first region, and second region. In some embodiments, there is a linker between any one of the first, second, third and fourth regions. In some embodiments, there is a linker between the first and fourth regions. In some embodiments, the effector protein is linked to an RDDP. In some embodiments, the RDDP comprises a reverse transcriptase.

In some embodiments, the third and fourth regions are 3′ of the first and second regions. In some embodiments, the order of the regions of the extended guide RNA from 5′ to 3′ is: first region, second region, third region, and fourth region. In some embodiments, there is a linker between the second and third regions.

### Protein Modification Activity

In some embodiments, a fusion partner provides enzymatic activity that modifies a protein associated with a target nucleic acid. The protein may be a histone, an RNA binding protein, or a DNA binding protein. Examples of such protein modification activities include: methyltransferase activity, such as that provided by a histone methyltransferase (HMT) (e.g., suppressor of variegation 3-9 homolog 1 (SUV39H1, also known as KMT1A), euchromatic histone lysine methyltransferase 2 (G9A, also known as KMT1C and EHMT2), SUV39H2, ESET/SETDB1, SET1A, SET1B, MLL1 to 5, ASH1, SYMD2, NSD1, DOT1L, Pr-SET7/8, SUV4-20H1, EZH2, RIZ1); demethylase activity such as that provided by a histone demethylase (e.g., Lysine Demethylase 1A (KDM1A also known as LSD1), JHDM2a/b, JMJD2A/JHDM3A, JMJD2B, JMJD2C/GASC1, JMJD2D, JARID1A/RBP2, JARID1B/PLU-1, JARID1C/SMCX, JARID1D/SMCY, UTX, JMJD3); acetyltransferase activity such as that provided by a histone acetylase transferase (e.g., catalytic core/fragment of the human acetyltransferase p300, GCN5, PCAF, CBP, TAF1, TIP60/PLIP, MOZ/MYST3, MORF/MYST4, HBO1/MYST2, HMOF/MYST1, SRC1, ACTR, P160, CLOCK); deacetylase activity such as that provided by a histone deacetylase (e.g., HDAC1, HDAC2, HDAC3, HDAC8, HDAC4, HDAC5, HDAC7, HDAC9, SIRT1, SIRT2, HDAC11); kinase activity; phosphatase activity; ubiquitin ligase activity; deubiquitinating activity; adenylation activity; deadenylation activity; SUMOylating activity; deSUMOylating activity; ribosylation activity; deribosylation activity; myristoylation activity; and demyristoylation activity.

### CRISPRa Fusions and CRISPRi Fusions

In some embodiments, fusion partners include, but are not limited to, a protein that directly and/or indirectly provides for increased or decreased transcription and/or translation of a target nucleic acid (e.g., a transcription activator or a fragment thereof, a protein or fragment thereof that recruits a transcription activator, a small molecule/drug-responsive transcription and/or translation regulator, a translation-regulating protein, etc.). In some embodiments, fusion partners that increase or decrease transcription include a transcription activator domain or a transcription repressor domain, respectively.

In some embodiments, fusion partners activate or increase expression of a target nucleic acid. Such fusion proteins comprising the described fusion partners and an effector protein may be referred to as CRISPRa fusions. In some embodiments, fusion partners increase expression of the target nucleic acid relative to its expression in the absence of the fusion effector protein. Relative expression, including transcription and RNA levels, may be assessed, quantified, and compared, e.g., by RT-qPCR. In some embodiments, fusion partners comprise a transcriptional activator. In general, a transcriptional activator refers to a polypeptide or a fragment thereof that can activate or increase transcription of a target nucleic acid molecule. In some embodiments, the transcriptional activators may promote transcription by: recruitment of other transcription factor proteins; modification of target DNA such as demethylation; recruitment of a DNA modifier; modulation of histones associated with target DNA; recruitment of a histone modifier such as those that modify acetylation and/or methylation of histones; or a combination thereof. In some embodiments, the fusion partner is a reverse transcriptase.

Non-limiting examples of fusion partners that promote or increase transcription include: transcriptional activators such as VP16, VP64, VP48, VP160, p65 subdomain (e.g., from NFkB), and activation domain of EDLL and/or TAL activation domain (e.g., for activity in plants); histone lysine methyltransferases such as SET1A, SET1B, MLL1 to 5, ASH1, SYMD2, NSD1; histone lysine demethylases such as JHDM2a/b, UTX, JMJD3; histone acetyltransferases such as GCN5, PCAF, CBP, p300, TAF1, TIP60/PLIP, MOZ/MYST3, MORF/MYST4, SRC1, ACTR, P160, CLOCK; and DNA demethylases such as Ten-Eleven Translocation (TET) dioxygenase 1 (TET1CD), TET1, DME, DML1, DML2, and ROS1; and functional domains thereof. Other non-limiting examples of suitable fusion partners include: proteins and protein domains responsible for stimulating translation (e.g., Staufen); proteins and protein domains responsible for (e.g., capable of) modulating translation (e.g., translation factors such as initiation factors, elongation factors, release factors, etc., e.g., eIF4G); proteins and protein domains responsible for stimulation of RNA splicing (e.g., Serine/Arginine-rich (SR) domains); and proteins and protein domains responsible for stimulating transcription (e.g., CDK7 and HIV Tat).

In some embodiments, fusions partners inhibit or reduce expression of a target nucleic acid. Such fusion proteins comprising described fusion partners and an effector protein may be referred to as CRISPRi fusions. In some embodiments, fusion partners reduce expression of the target nucleic acid relative to its expression in the absence of the fusion effector protein. Relative expression, including transcription and RNA levels, may be assessed, quantified, and compared, e.g., by RT-qPCR. In some embodiments, fusion partners may comprise a transcriptional repressor. In some embodiments, the transcriptional repressors may inhibit transcription by: recruitment of other transcription factor proteins; modification of target DNA such as methylation; recruitment of a DNA modifier; modulation of histones associated with target DNA; recruitment of a histone modifier such as those that modify acetylation and/or methylation of histones; or a combination thereof.

In some embodiments, the guide nucleic acids disclosed herein can be used in combination with a fusion protein for epigenetic modification of the APOC3, the PCSK9, or the ANGPTL3 genes. In some embodiments, the fusion protein comprises an effector protein and a methyltransferase. In some embodiments, the fusion protein further comprises a KRAB domain. In some embodiments, the methyltransferase is selected from M.HhaI, DNMT1, DNMT3A, DNMT3B, DNMT3L, and a combination thereof. In some embodiments, the methyltransferase is selected from DNMT3A, DNMT3L, and a combination thereof. In some embodiments, the methyltransferase is DNMT3L. In some embodiments, the fusion protein does not comprise DNMT3A. In some embodiments, the effector protein is CasPhi.12 or a variant thereof, and the guide nucleic acid comprises a sequence that is at least at least 70%, at least 80%, at least 90%, at least 91%, at least 92%, at least 93%, at least 94%, at least 95%, at least 96%, at least 97%, or at least 99%, or 100% identical to any one of the sequences of SEQ ID NOs: 1400-1569. In some embodiments, the effector protein is CasM.265466 or a variant thereof, and the guide nucleic acid comprises a sequence that is at least at least 70%, at least 80%, at least 90%, at least 91%, at least 92%, at least 93%, at least 94%, at least 95%, at least 96%, at least 97%, or at least 99%, or 100% identical to any one of the sequences of SEQ ID NOs: 1570-1969.

Non-limiting examples of fusion partners that decrease or inhibit transcription include: transcriptional repressors such as the Krüppel associated box (KRAB or SKD); KOX1 repression domain; the Mad mSIN3 interaction domain (SID); the ERF repressor domain (ERD), the SRDX repression domain (e.g., for repression in plants); histone lysine methyltransferases such as Pr-SET7/8, SUV4-20H1, RIZ1, and the like; histone lysine demethylases such as JMJD2A/JHDM3A, JMJD2B, JMJD2C/GASC1, JMJD2D, JARID1A/RBP2, JARID1B/PLU-1, JARID1C/SMCX, JARID1D/SMCY; histone lysine deacetylases such as HDAC1, HDAC2, HDAC3, HDAC8, HDAC4, HDAC5, HDAC7, HDAC9, SIRT1, SIRT2, HDAC11; DNA methylases such as HhaI DNA m5c-methyltransferase (M.HhaI), DNA methyltransferase 1 (DNMT1), DNA methyltransferase 3a (DNMT3a), DNA methyltransferase 3b (DNMT3b), METI, DRM3 (plants), ZMET2, CMT1, CMT2 (plants); and periphery recruitment elements such as Lamin A, and Lamin B; and functional domains thereof. Other non-limiting examples of suitable fusion partners include: proteins and protein domains responsible for repressing translation (e.g., Ago2 and Ago4); proteins and protein domains responsible for repression of RNA splicing (e.g., PTB, Sam68, and hnRNP A1); proteins and protein domains responsible for reducing the efficiency of transcription (e.g., FUS (TLS)).

In some embodiments, fusion proteins are targeted by a guide nucleic acid (e.g., guide RNA) to a specific location in a target nucleic acid and exert locus-specific regulation such as blocking RNA polymerase binding to a promoter (which selectively inhibits transcription activator function), and/or changes a local chromatin status (e.g., when a fusion sequence is used that edits the target nucleic acid or modifies a protein associated with the target nucleic acid). In some embodiments, the modifications are transient (e.g., transcription repression or activation). In some embodiments, the modifications are inheritable. For example, epigenetic modifications made to a target nucleic acid, or to proteins associated with the target nucleic acid, e.g., nucleosomal histones, in a cell, can be observed in a successive generation.

In some embodiments, fusion partner comprises an RNA splicing factor. The RNA splicing factor may be used (in whole or as fragments thereof) for modular organization, with separate sequence-specific RNA binding modules and splicing effector domains. In some embodiments, the RNA splicing factors comprise members of the Serine/Arginine-rich (SR) protein family containing N-terminal RNA recognition motifs (RRMs) that bind to exonic splicing enhancers (ESEs) in pre-mRNAs and C-terminal RS domains that promote exon inclusion. In some embodiments, a hnRNP protein hnRNP Al binds to exonic splicing silencers (ESSs) through its RRM domains and inhibits exon inclusion through a C-terminal Glycine-rich domain. In some embodiments, the RNA splicing factors may regulate alternative use of splice site (ss) by binding to regulatory sequences between two alternative sites. For example, in some embodiments, ASF/SF2 may recognize ESEs and promote the use of intron proximal sites, whereas hnRNP Al may bind to ESSs and shift splicing towards the use of intron distal sites. One application for such factors is to generate ESFs that modulate alternative splicing of endogenous genes, particularly disease associated genes. For example, Bcl-x pre-mRNA produces two splicing isoforms with two alternative 5′ splice sites to encode proteins of opposite functions. Long splicing isoform Bcl-xL is a potent apoptosis inhibitor expressed in long-lived postmitotic cells and is up-regulated in many cancer cells, protecting cells against apoptotic signals. Short isoform Bcl-xS is a pro-apoptotic isoform and expressed at high levels in cells with a high turnover rate (e.g., developing lymphocytes). A ratio of the two Bcl-x splicing isoforms is regulated by multiple c\acute{ω}-elements that are located in either core exon region or exon extension region (i.e., between the two alternative 5′ splice sites). For more examples, see WO2010075303, which is hereby incorporated by reference in its entirety.

### Recombinases

In some embodiments, fusion partners comprise a recombinase. In some embodiments, effector proteins described herein are linked with the recombinase. In some embodiments, the effector proteins have reduced nuclease activity or no nuclease activity. In some embodiments, the recombinase is a site-specific recombinase.

In some embodiments, a catalytically inactive effector protein is linked with a recombinase, wherein the recombinase can be a site-specific recombinase. Such polypeptides can be used for site-directed transgene insertion. Non-limiting examples of site-specific recombinases include a tyrosine recombinase (e.g., Cre, Flp or lambda integrase), a serine recombinase (e.g., gamma-delta resolvase, Tn3 resolvase, Sin resolvase, Gin invertase, Hin invertase, Tn5044 resolvase, IS607 transposase and integrase), or mutants or variants thereof. In some embodiments, the recombinase is a serine recombinase. Non-limiting examples of serine recombinases include gamma-delta resolvase, Tn3 resolvase, Sin resolvase, Gin invertase, Hin invertase, Tn5044 resolvase, IS607 transposase, and IS607 integrase. In some embodiments, the site-specific recombinase is an integrase. Non-limiting examples of integrases include: Bxb1, wBeta, BL3, phiR4, A118, TG1, MR11, phi370, SPBc, TP901-1, phiRV, FC1, K38, phiBT1, and phiC31. Further discussion and examples of suitable recombinase fusion partners are described in U.S. Pat. No. 10,975,392, which is incorporated herein by reference in its entirety. In some embodiments, the fusion protein comprises a linker that links the recombinase to the Cas-CRISPR domain of the effector protein. In some embodiments, the linker is The-Ser.

### 5. Exemplary Systems

In some embodiments, the present disclosure provides a system comprising (1) a guide RNA or a polynucleotide encoding the same, wherein the guide RNA comprises a spacer sequence that is capable of hybridizing to a target nucleic acid sequence in a gene selected from APOC3, PCSK9, and ANGPTL3; and (2) an effector protein or fusion protein thereof or a polynucleotide encoding the same.

### Exemplary APOC3 Systems

In some embodiments, the present disclosure provides a system comprising (1) a guide RNA or a polynucleotide encoding the same, wherein the guide RNA comprises a spacer sequence that is capable of hybridizing to a target nucleic acid sequence in the APOC3 gene; and (2) an effector protein or fusion protein thereof or a polynucleotide encoding the same.

In some embodiments, the effector protein comprises an amino acid sequence that is at least 90%, at least, 91%, at least 92%, at least 93%, at least 94%, at least 95%, at least 96%, at least 97%, at least 98%, or at least 99% identical to any one of the sequences recited in TABLEs 15, 18, and 19, and the guide RNA comprises (a) a repeat sequence that is at least 90%, at least, 91%, at least 92%, at least 93%, at least 94%, at least 95%, at least 96%, at least 97%, at least 98%, or at least 99% identical to a sequence selected from any one of SEQ ID NOs: 16 and 38-43 and (b) a spacer sequence that is at least 90%, at least, 91%, at least 92%, at least 93%, at least 94%, at least 95%, at least 96%, at least 97%, at least 98%, or at least 99% identical a sequence selected from to any one of SEQ ID NOs: 1-15, 67-72, 207, 804-805, and 830-999. In some embodiments, the guide RNA sequence is at least 90%, at least, 91%, at least 92%, at least 93%, at least 94%, at least 95%, at least 96%, at least 97%, at least 98%, or at least 99% identical to a sequence selected from any one of SEQ ID NOs: 17-31, 73-78, 491, 815-816, and 1400-1569.

In some embodiments, the effector protein comprises any one of the sequences recited in TABLEs 15, 18, and 19, and the guide RNA comprises (a) a repeat sequence comprising any one of SEQ ID NOs: 16 and 38-43 and (b) a spacer sequence comprising any one of SEQ ID NOs: 1-15, 67-72, 207, 804-805, and 830-999. In some embodiments, the guide RNA sequence comprises any one of SEQ ID NOs: 17-31, 73-78, 491, 815-816, and 1400-1569.

In some embodiments, the effector protein consists of a sequence recited in TABLEs 15, 18, or 19, and the guide RNA consists of (a) a repeat sequence consisting of a sequence selected from any one of SEQ ID NOs: 16 and 38-43 and (b) a spacer sequence consisting of a sequence selected from any one of SEQ ID NOs: 1-15, 67-72, 207, 804-805, or 830-999. In some embodiments, the guide RNA sequence consists of a sequence selected from any one of SEQ ID NOs: 17-31, 73-78, 491, 815-816, and 1400-1569.

In some embodiments, the effector protein comprises an amino acid sequence that is at least 90%, at least, 91%, at least 92%, at least 93%, at least 94%, at least 95%, at least 96%, at least 97%, at least 98%, or at least 99% identical to a sequence selected from any one of SEQ ID NOs: 32, 34, 794, and 2090, and the guide RNA comprises (a) a repeat sequence that is at least 90%, at least, 91%, at least 92%, at least 93%, at least 94%, at least 95%, at least 96%, at least 97%, at least 98%, or at least 99% identical to a sequence selected any one of SEQ ID NOs: 16 and 38-43 and (b) a spacer sequence that is at least 90%, at least, 91%, at least 92%, at least 93%, at least 94%, at least 95%, at least 96%, at least 97%, at least 98%, or at least 99% identical a sequence selected from to any one of SEQ ID NOs: 1-15, 67-72, 207, 804-805, and 830-999. In some embodiments, the guide RNA sequence is at least 90%, at least, 91%, at least 92%, at least 93%, at least 94%, at least 95%, at least 96%, at least 97%, at least 98%, or at least 99% identical a sequence selected from to any one of SEQ ID NOs: 17-31, 73-78, 491, 815-816, and 1400-1569.

In some embodiments, the effector protein comprises any one of SEQ ID NOs: 32, 34, 794, and 2090, and the guide RNA comprises (a) a repeat sequence comprising any one of SEQ ID NOs: 16 and 38-43 and (b) a spacer sequence comprising any one of SEQ ID NOs: 1-15, 67-72, 207, 804-805, and 830-999. In some embodiments, the guide RNA sequence comprises any one of SEQ ID NOs: 17-31, 73-78, 491, 815-816, and 1400-1569.

In some embodiments, the effector protein consists of a sequence selected from any one of SEQ ID NOs: 32, 34, 794, or 2090, and the guide RNA consists of (a) a repeat sequence consisting of a sequence selected from any one of SEQ ID NOs: 16 or 38-43 and (b) a spacer sequence consisting of a sequence selected from any one of SEQ ID NOs: 1-15, 67-72, 207, 804-805, or 830-999. In some embodiments, the guide RNA sequence consists of a sequence selected from any one of SEQ ID NOs: 17-31, 73-78, 491, 815-816, or 1400-1569.

In some embodiments, the effector protein comprises an amino acid sequence that is at least 90%, at least, 91%, at least 92%, at least 93%, at least 94%, at least 95%, at least 96%, at least 97%, at least 98%, or at least 99% identical to a sequence selected from any one of SEQ ID NOs: 32, 34, 794, and 2090, wherein the effector protein is fused to a KRAB domain, a methyltransferase, or a combination thereof, and the guide RNA comprises (a) a repeat sequence that is at least 90%, at least, 91%, at least 92%, at least 93%, at least 94%, at least 95%, at least 96%, at least 97%, at least 98%, or at least 99% identical to a sequence selected from any one of SEQ ID NOs: 16 and 38-43 and (b) a spacer sequence that is at least 90%, at least, 91%, at least 92%, at least 93%, at least 94%, at least 95%, at least 96%, at least 97%, at least 98%, or at least 99% identical to a sequence selected from any one of SEQ ID NOs: 830-999. In some embodiments, the guide RNA sequence is at least 90%, at least, 91%, at least 92%, at least 93%, at least 94%, at least 95%, at least 96%, at least 97%, at least 98%, or at least 99% identical to a sequence selected from any one of SEQ ID NOs: 1400-1569.

In some embodiments, the effector protein comprises any one of SEQ ID NOs: 32, 34, 794, and 2090, wherein the effector protein is fused to a KRAB domain, a methyltransferase, or a combination thereof, and the guide RNA comprises (a) a repeat sequence comprising any one of SEQ ID NOs: 16 and 38-43 and (b) a spacer sequence comprising any one of SEQ ID NOs: 830-999. In some embodiments, the guide RNA sequence comprises any one of SEQ ID NOs: 1400-1569.

In some embodiments, the effector protein consists of a sequence selected from any one of SEQ ID NOs: 32, 34, 794, or 2090, wherein the effector protein is fused to a KRAB domain, a methyltransferase, or a combination thereof, and the guide RNA consists of (a) a repeat sequence consisting of a sequence selected from any one of SEQ ID NOs: 16 or 38-43 and (b) a spacer sequence consisting of a sequence selected from any one of SEQ ID NOs: 830-999. In some embodiments, the guide RNA sequence consists of a sequence selected from any one of SEQ ID NOs: 1400-1569.

In some embodiments, the effector protein comprises an amino acid sequence that is at least 90%, at least, 91%, at least 92%, at least 93%, at least 94%, at least 95%, at least 96%, at least 97%, at least 98%, or at least 99% identical to a sequence selected from any one of SEQ ID NOs: 32, 34, 794, and 2090, and the guide RNA comprises (a) a repeat sequence that is at least 90%, at least, 91%, at least 92%, at least 93%, at least 94%, at least 95%, at least 96%, at least 97%, at least 98%, or at least 99% identical SEQ ID NO: 39 and (b) a spacer sequence that is at least 90%, at least, 91%, at least 92%, at least 93%, at least 94%, at least 95%, at least 96%, at least 97%, at least 98%, or at least 99% identical to SEQ ID NO: 10. In some embodiments, the guide RNA sequence is at least 90%, at least, 91%, at least 92%, at least 93%, at least 94%, at least 95%, at least 96%, at least 97%, at least 98%, or at least 99% identical to any one of SEQ ID NO: 26.

In some embodiments, the effector protein comprises any one of SEQ ID NOs: 32, 34, 794, and 2090, and the guide RNA comprises (a) a repeat sequence comprising SEQ ID NO: 39 and (b) a spacer sequence comprising SEQ ID NO: 10. In some embodiments, the guide RNA sequence comprises SEQ ID NO: 26.

In some embodiments, the effector protein consists of any one of SEQ ID NOs: 32, 34, 794, or 2090, and the guide RNA consists of (a) a repeat sequence consisting of SEQ ID NO: 39 and (b) a spacer sequence consisting of SEQ ID NO: 10. In some embodiments, the guide RNA sequence consists of SEQ ID NO: 26.

In some embodiments, the effector protein comprises an amino acid sequence that is at least 90%, at least, 91%, at least 92%, at least 93%, at least 94%, at least 95%, at least 96%, at least 97%, at least 98%, or at least 99% identical to a sequence selected from any one of SEQ ID NOs: 32, 34, 794, and 2090, and the guide RNA comprises (a) a repeat sequence that is at least 90%, at least, 91%, at least 92%, at least 93%, at least 94%, at least 95%, at least 96%, at least 97%, at least 98%, or at least 99% identical SEQ ID NO: 39 and (b) a spacer sequence that is at least 90%, at least, 91%, at least 92%, at least 93%, at least 94%, at least 95%, at least 96%, at least 97%, at least 98%, or at least 99% identical to SEQ ID NO: 71. In some embodiments, the guide RNA sequence is at least 90%, at least, 91%, at least 92%, at least 93%, at least 94%, at least 95%, at least 96%, at least 97%, at least 98%, or at least 99% identical to any one of SEQ ID NO: 77.

In some embodiments, the effector protein comprises any one of SEQ ID NOs: 32, 34, 794, and 2090, and the guide RNA consists of (a) a repeat sequence consisting of SEQ ID NO: 39 and (b) a spacer sequence consisting of SEQ ID NO: 71. In some embodiments, the guide RNA sequence consists of SEQ ID NO: 77.

In some embodiments, the effector protein consists of a sequence selected from any one of SEQ ID NOs: 32, 34, 794, or 2090, and the guide RNA consists of (a) a repeat sequence consisting of SEQ ID NO: 39 and (b) a spacer sequence consisting of SEQ ID NO: 71. In some embodiments, the guide RNA sequence consists of SEQ ID NO: 77.

In some embodiments, the effector protein comprises an amino acid sequence that is at least 90%, at least, 91%, at least 92%, at least 93%, at least 94%, at least 95%, at least 96%, at least 97%, at least 98%, or at least 99% identical to a sequence selected from any one of TABLEs 15, 16, and 17, and the guide RNA comprises (a) a repeat sequence that is at least 90%, at least, 91%, at least 92%, at least 93%, at least 94%, at least 95%, at least 96%, at least 97%, at least 98%, or at least 99% identical to a sequence selected from any one of SEQ ID NO: 488, and (b) a spacer sequence that is at least 90%, at least, 91%, at least 92%, at least 93%, at least 94%, at least 95%, at least 96%, at least 97%, at least 98%, or at least 99% identical to a sequence selected from any one of SEQ ID NOs: 209-299, 823-825, 1000-1399, 2018-2026, and 2084-2086. In some embodiments, the system further comprises an (c) intermediary sequence that is at least 90%, at least, 91%, at least 92%, at least 93%, at least 94%, at least 95%, at least 96%, at least 97%, at least 98%, or at least 99% identical to SEQ ID NO: 489 or (d) a handle sequence that is at least 90%, at least, 91%, at least 92%, at least 93%, at least 94%, at least 95%, at least 96%, at least 97%, at least 98%, or at least 99% identical to SEQ ID NO: 490. In some embodiments, the guide RNA sequence is at least 90%, at least, 91%, at least 92%, at least 93%, at least 94%, at least 95%, at least 96%, at least 97%, at least 98%, or at least 99% identical to a sequence selected from any one of SEQ ID NOs: 494-584, 826-828, 1570-1969, 2075-2083, and 2087-2089.

In some embodiments, the effector protein comprises any one of the sequences recited in TABLEs 15, 16, and 17, and the guide RNA comprises (a) a repeat sequence comprising SEQ ID NO: 488 and (b) a spacer sequence selected from any one of SEQ ID NOs: 209-299, 823-825, 1000-1399, 2018-2026, and 2084-2086. In some embodiments, the system further comprises (c) an intermediary sequence comprising SEQ ID NO: 489 or (d) a handle sequence comprising SEQ ID NO: 490. In some embodiments, the guide RNA sequence comprises any one of SEQ ID NOs: 494-584, 826-828, 1570-1969, 2075-2083, and 2087-2089.

In some embodiments, the effector protein consists of any one of the sequences recited in TABLEs 15, 16, or 17, and (2) a guide RNA consists of (a) a repeat sequence consisting of SEQ ID NO: 488 and a spacer sequence consisting of (b) a sequence selected from any one of SEQ ID NOs: 209-299, 823-825, 1000-1399, 2018-2026, or 2084-2086. In some embodiments, the system further comprises (c) an intermediary sequence consisting of SEQ ID NO: 489 or (d) a handle sequence consisting of SEQ ID NO: 490. In some embodiments, the guide RNA sequence consists of a sequence selected from any one of SEQ ID NOs: 494-584, 826-828, 1570-1969, 2075-2083, or 2087-2089.

In some embodiments, the effector protein comprises an amino acid sequence that is at least 90%, at least, 91%, at least 92%, at least 93%, at least 94%, at least 95%, at least 96%, at least 97%, at least 98%, or at least 99% identical to a sequence selected from any one of SEQ ID NOs: 773, 775, and 793, and the guide RNA comprises (a) a repeat sequence that is at least 90%, at least, 91%, at least 92%, at least 93%, at least 94%, at least 95%, at least 96%, at least 97%, at least 98%, or at least 99% identical to SEQ ID NO: 488, and (b) a spacer sequence that is at least 90%, at least, 91%, at least 92%, at least 93%, at least 94%, at least 95%, at least 96%, at least 97%, at least 98%, or at least 99% identical to a sequence selected from any one of SEQ ID NOs: 209-299, 823-825, 1000-1399, 2018-2026, and 2084-2086. In some embodiments, the system further comprises (c) an intermediary sequence that is at least 90%, at least, 91%, at least 92%, at least 93%, at least 94%, at least 95%, at least 96%, at least 97%, at least 98%, or at least 99% identical to SEQ ID NO: 489 or (d) a handle sequence that is at least 90%, at least, 91%, at least 92%, at least 93%, at least 94%, at least 95%, at least 96%, at least 97%, at least 98%, or at least 99% identical to SEQ ID NO: 490. In some embodiments, the guide RNA sequence is at least 90%, at least, 91%, at least 92%, at least 93%, at least 94%, at least 95%, at least 96%, at least 97%, at least 98%, or at least 99% identical to a sequence selected from any one of SEQ ID NOs: 494-584, 826-828, 1570-1969, 2075-2083, and 2087-2089.

In some embodiments, the effector protein comprises a sequence selected from any one of SEQ ID NOs: 773, 775, and 793, and the guide RNA comprises (a) a repeat sequence comprising SEQ ID NO: 488 and (b) a spacer sequence selected from any one of SEQ ID NOs: 209-299, 823-825, 1000-1399, 2018-2026, and 2084-2086. In some embodiments, the system further comprises (c) an intermediary sequence comprising SEQ ID NO: 489 or (d) a handle sequence comprising SEQ ID NO: 490. In some embodiments, the guide RNA sequence comprises any one of SEQ ID NOs: 494-584, 826-828, 1570-1969, 2075-2083, and 2087-2089.

In some embodiments, the effector protein consists of a sequence selected from any one of SEQ ID NOs: 773, 775, or 793, and the guide RNA consists of (a) a repeat sequence consisting of SEQ ID NO: 488 and (b) a spacer sequence consisting of a sequence selected from any one of SEQ ID NOs: 209-299, 823-825, 1000-1399, 2018-2026, or 2084-2086. In some embodiments, the system further comprises (c) an intermediary sequence consisting of SEQ ID NO: 489 or (d) a handle sequence consisting of SEQ ID NO: 490. In some embodiments, the guide RNA sequence consists of a sequence selected from any one of SEQ ID NOs: 494-584, 826-828, 1570-1969, 2075-2083, or 2087-2089.

In some embodiments, the effector protein comprises an amino acid sequence that is at least 90%, at least, 91%, at least 92%, at least 93%, at least 94%, at least 95%, at least 96%, at least 97%, at least 98%, or at least 99% identical to a sequence selected from any one of SEQ ID NOs: 773, 775, and 793, wherein the effector protein is fused to a KRAB domain, a methyltransferase, or a combination thereof, and the guide RNA comprises (a) a repeat sequence that is at least 90%, at least, 91%, at least 92%, at least 93%, at least 94%, at least 95%, at least 96%, at least 97%, at least 98%, or at least 99% identical to SEQ ID NO: 488, and (b) a spacer sequence that is at least 90%, at least, 91%, at least 92%, at least 93%, at least 94%, at least 95%, at least 96%, at least 97%, at least 98%, or at least 99% identical to a sequence selected from any one of SEQ ID NOs: 1000-1399. In some embodiments, the system further comprises (c) an intermediary sequence that is at least 90%, at least, 91%, at least 92%, at least 93%, at least 94%, at least 95%, at least 96%, at least 97%, at least 98%, or at least 99% identical to SEQ ID NO: 489 and (d) a handle sequence that is at least 90%, at least, 91%, at least 92%, at least 93%, at least 94%, at least 95%, at least 96%, at least 97%, at least 98%, or at least 99% identical to SEQ ID NO: 490. In some embodiments, the guide RNA sequence is at least 90%, at least, 91%, at least 92%, at least 93%, at least 94%, at least 95%, at least 96%, at least 97%, at least 98%, or at least 99% identical to a sequence selected from any one of SEQ ID NOs: 1570-1969.

In some embodiments, the effector protein comprises a sequence selected from any one of SEQ ID NOs: 773, 775, and 793, wherein the effector protein is fused to a KRAB domain, a methyltransferase, or a combination thereof, and the guide RNA comprises (a) a repeat sequence comprising SEQ ID NO: 488 and (b) a spacer sequence selected from any one of SEQ ID NOs: 1000-1399. In some embodiments, the system further comprises (c) an intermediary sequence comprising SEQ ID NO: 489 or (d) a handle sequence comprising SEQ ID NO: 490. In some embodiments, the guide RNA sequence comprises any one of SEQ ID NOs: 1570-1969.

In some embodiments, the effector protein consists of a sequence selected from any one of SEQ ID NOs: 773, 775, or 793, wherein the effector protein is fused to a KRAB domain, a methyltransferase, or a combination thereof, and the guide RNA consists of (a) a repeat sequence consisting of SEQ ID NO: 488 and (b) a spacer sequence consisting a sequence selected from of any one of SEQ ID NOs: 1000-1399. In some embodiments, the system further comprises (c) an intermediary sequence consisting of SEQ ID NO: 489 or (d) a handle sequence consisting of SEQ ID NO: 490. In some embodiments, the guide RNA sequence consists of a sequence selected from any one of SEQ ID NOs: 1570-1969.

In some embodiments, the effector protein comprises an amino acid sequence that is at least 90%, at least, 91%, at least 92%, at least 93%, at least 94%, at least 95%, at least 96%, at least 97%, at least 98%, or at least 99% identical a sequence selected from to any one of SEQ ID NOs: 773, 775, and 793, wherein the effector protein is fused to a base editing enzyme, and the guide RNA comprises (a) a repeat sequence that is at least 90%, at least, 91%, at least 92%, at least 93%, at least 94%, at least 95%, at least 96%, at least 97%, at least 98%, or at least 99% identical to SEQ ID NO: 488, and (b) a spacer sequence that is at least 90%, at least, 91%, at least 92%, at least 93%, at least 94%, at least 95%, at least 96%, at least 97%, at least 98%, or at least 99% identical to a sequence selected from any one of SEQ ID NOs: 2018-2026. In some embodiments, the system further comprises (c) an intermediary sequence that is at least 90%, at least, 91%, at least 92%, at least 93%, at least 94%, at least 95%, at least 96%, at least 97%, at least 98%, or at least 99% identical to SEQ ID NO: 489 and (d) a handle sequence that is at least 90%, at least, 91%, at least 92%, at least 93%, at least 94%, at least 95%, at least 96%, at least 97%, at least 98%, or at least 99% identical to SEQ ID NO: 490. In some embodiments, the guide RNA sequence is at least 90%, at least, 91%, at least 92%, at least 93%, at least 94%, at least 95%, at least 96%, at least 97%, at least 98%, or at least 99% identical to a sequence selected from any one of SEQ ID NOs: 2075-2083.

In some embodiments, the effector protein comprises a sequence selected from any one of SEQ ID NOs: 773, 775, and 793, wherein the effector protein is fused to a base editing enzyme, and the guide RNA comprises (a) a repeat sequence comprising SEQ ID NO: 488 and (b) a spacer sequence selected from any one of SEQ ID NOs: 2018-2026. In some embodiments, the system further comprises (c) an intermediary sequence comprising SEQ ID NO: 489 or (d) a handle sequence comprising SEQ ID NO: 490. In some embodiments, the guide RNA sequence comprising a sequence selected from any one of SEQ ID NOs: 2075-2083.

In some embodiments, the effector protein consists of a sequence selected from any one of SEQ ID NOs: 773, 775, or 793, wherein the effector protein is fused to a base editing enzyme, and the guide RNA consists of (a) a repeat sequence consisting of SEQ ID NO: 488 and (b) a spacer sequence consisting of a sequence selected from any one of SEQ ID NOs: 2018-2026. In some embodiments, the system further comprises (c) an intermediary sequence comprising SEQ ID NO: 489 or (d) a handle sequence comprising SEQ ID NO: 490. In some embodiments, the guide RNA sequence consists of a sequence selected from any one of SEQ ID NOs: 2075-2083.

### Exemplary PCSK9 Systems

In some embodiments, the present disclosure provides a system comprising (1) a guide RNA or a polynucleotide encoding the same, wherein the guide RNA comprises a spacer sequence that is capable of hybridizing to a target nucleic acid sequence in the PCSK9 gene; and (2) an effector protein or fusion protein thereof or a polynucleotide encoding the same.

In some embodiments, the effector protein comprises an amino acid sequence that is at least 90%, at least, 91%, at least 92%, at least 93%, at least 94%, at least 95%, at least 96%, at least 97%, at least 98%, or at least 99% identical to any one of the sequences recited in TABLEs 15, 18, and 19, and the guide RNA comprises (a) a repeat sequence that is at least 90%, at least, 91%, at least 92%, at least 93%, at least 94%, at least 95%, at least 96%, at least 97%, at least 98%, or at least 99% identical to a sequence selected from any one of SEQ ID NOs: 16 and 38-43 and (b) a spacer sequence that is at least 90%, at least, 91%, at least 92%, at least 93%, at least 94%, at least 95%, at least 96%, at least 97%, at least 98%, or at least 99% identical to a sequence selected from any one of SEQ ID NOs: 79-140, 208, 799-803, and 809. In some embodiments, the guide RNA sequence is at least 90%, at least, 91%, at least 92%, at least 93%, at least 94%, at least 95%, at least 96%, at least 97%, at least 98%, or at least 99% identical to a sequence selected from any one of SEQ ID NOs: 141-202, 492-493, 810-814, and 820.

In some embodiments, the effector protein comprises any one of the sequences recited in TABLEs 15, 18, and 19, and the guide RNA comprises (a) a repeat sequence comprising a sequence selected from any one of SEQ ID NOs: 16 and 38-43 and (b) a spacer sequence selected from any one of SEQ ID NOs: 79-140, 208, 799-803, and 809. In some embodiments, the guide RNA sequence comprises any one of SEQ ID NOs: 141-202, 492-493, 810-814, and 820.

In some embodiments, the effector protein consists of any one of the sequences recited in TABLEs 15, 18, or 19, and the guide RNA consists of (a) a repeat sequence consisting of a sequence selected from any one of SEQ ID NOs: 16 or 38-43 and (b) a spacer sequence consisting of a sequence selected from any one of SEQ ID NOs: 79-140, 208, 799-803, or 809. In some embodiments, the guide RNA sequence consists of a sequence selected from any one of SEQ ID NOs: 141-202, 492-493, 810-814, or 820.

In some embodiments, the effector protein comprises an amino acid sequence that is at least 90%, at least, 91%, at least 92%, at least 93%, at least 94%, at least 95%, at least 96%, at least 97%, at least 98%, or at least 99% identical to a sequence selected from any one of SEQ ID NOs: 32, 34, 794, and 2090, and (2) a guide RNA comprises (a) a repeat sequence that is at least 90%, at least, 91%, at least 92%, at least 93%, at least 94%, at least 95%, at least 96%, at least 97%, at least 98%, or at least 99% identical to a sequence selected from any one of SEQ ID NOs: 16 and 38-43 and (b) a spacer sequence that is at least 90%, at least, 91%, at least 92%, at least 93%, at least 94%, at least 95%, at least 96%, at least 97%, at least 98%, or at least 99% identical to a sequence selected from any one of SEQ ID NOs: 79-140, 208, 799-803, and 809. In some embodiments, the guide RNA sequence is at least 90%, at least, 91%, at least 92%, at least 93%, at least 94%, at least 95%, at least 96%, at least 97%, at least 98%, or at least 99% identical to a sequence selected from any one of SEQ ID NOs: 141-202, 492-493, 810-814, and 820.

In some embodiments, the effector protein comprises a sequence selected from any one of SEQ ID NOs: 32, 34, 794, and 2090, and the guide RNA comprises (a) a repeat sequence comprising a sequence selected from any one of SEQ ID NOs: 16 and 38-43 and (b) a spacer sequence comprising a sequence selected from any one of SEQ ID NOs: 79-140, 208, 799-803, and 809. In some embodiments, the guide RNA sequence comprises any one of SEQ ID NOs: 141-202, 492-493, 810-814, and 820.

In some embodiments, the effector protein consists of any one of SEQ ID NOs: 32, 34, 794, or 2090, and the guide RNA consists of (a) a repeat sequence consisting of a sequence selected from any one of SEQ ID NOs: 16 or 38-43 and (b) a spacer sequence consisting of a sequence selected from any one of SEQ ID NOs: 79-140, 208, 799-803, or 809. In some embodiments, the guide RNA sequence consists of a sequence selected from any one of SEQ ID NOs: 141-202, 492-493, 810-814, or 820.

In some embodiments, the effector protein comprises an amino acid sequence that is at least 90%, at least, 91%, at least 92%, at least 93%, at least 94%, at least 95%, at least 96%, at least 97%, at least 98%, or at least 99% identical to any one of the sequences recited in TABLEs 15, 16, and 17, and the guide RNA comprises (a) a repeat sequence that is at least 90%, at least, 91%, at least 92%, at least 93%, at least 94%, at least 95%, at least 96%, at least 97%, at least 98%, or at least 99% identical to SEQ ID NO: 488, and (b) a spacer sequence that is at least 90%, at least, 91%, at least 92%, at least 93%, at least 94%, at least 95%, at least 96%, at least 97%, at least 98%, or at least 99% identical to a sequence selected from any one of SEQ ID NOs: 300-487, 822, and 1970-1995. In some embodiments, the system further comprises (c) an intermediary sequence that is at least 90%, at least, 91%, at least 92%, at least 93%, at least 94%, at least 95%, at least 96%, at least 97%, at least 98%, or at least 99% identical to SEQ ID NO: 489 and (d) a handle sequence that is at least 90%, at least, 91%, at least 92%, at least 93%, at least 94%, at least 95%, at least 96%, at least 97%, at least 98%, or at least 99% identical to SEQ ID NO: 490. In some embodiments, the guide RNA sequence is at least 90%, at least, 91%, at least 92%, at least 93%, at least 94%, at least 95%, at least 96%, at least 97%, at least 98%, or at least 99% identical to a sequence selected from any one of SEQ ID NOs: 585-772, 829, and 2027-2052.

In some embodiments, the effector protein comprises any one of the sequences recited in TABLEs 15, 16, and 17, and the guide RNA comprises (a) a repeat sequence comprising SEQ ID NO: 488 and (b) a spacer sequence selected from any one of SEQ ID NOs: 300-487, 822, and 1970-1995. In some embodiments, the system further comprises (c) an intermediary sequence comprising SEQ ID NO: 489 and (d) a handle sequence comprising SEQ ID NO: 490. In some embodiments, the guide RNA sequence comprises any one of SEQ ID NOs: 585-772, 829, and 2027-2052.

In some embodiments, the effector protein consists of any one of the sequences recited in TABLEs 15, 16, or 17, and the guide RNA consists of (a) a repeat sequence consisting of SEQ ID NO: 488 and (b) a spacer sequence consisting of a sequence selected from any one of SEQ ID NOs: 300-487, 822, or 1970-1995. In some embodiments, the system further comprises (c) an intermediary sequence consisting of SEQ ID NO: 489 or (d) a handle sequence consisting of SEQ ID NO: 490. In some embodiments, the guide RNA sequence consists of a sequence selected from any one of SEQ ID NOs: 585-772, 829, or 2027-2052.

In some embodiments, the effector protein comprises an amino acid sequence that is at least 90%, at least, 91%, at least 92%, at least 93%, at least 94%, at least 95%, at least 96%, at least 97%, at least 98%, or at least 99% identical to a sequence selected from any one of SEQ ID NOs: 773, 775, and 793, and the guide RNA comprises (a) a repeat sequence that is at least 90%, at least, 91%, at least 92%, at least 93%, at least 94%, at least 95%, at least 96%, at least 97%, at least 98%, or at least 99% identical to SEQ ID NO: 488, and (b) a spacer sequence that is at least 90%, at least, 91%, at least 92%, at least 93%, at least 94%, at least 95%, at least 96%, at least 97%, at least 98%, or at least 99% identical to a sequence selected from any one of SEQ ID NOs: 300-487, 822, and 1970-1995. In some embodiments, the system further comprises (c) an intermediary sequence that is at least 90%, at least, 91%, at least 92%, at least 93%, at least 94%, at least 95%, at least 96%, at least 97%, at least 98%, or at least 99% identical to SEQ ID NO: 489 or (d) a handle sequence that is at least 90%, at least, 91%, at least 92%, at least 93%, at least 94%, at least 95%, at least 96%, at least 97%, at least 98%, or at least 99% identical to SEQ ID NO: 490. In some embodiments, the guide RNA sequence is at least 90%, at least, 91%, at least 92%, at least 93%, at least 94%, at least 95%, at least 96%, at least 97%, at least 98%, or at least 99% identical to a sequence selected from any one of SEQ ID NOs: 585-772, 829, and 2027-2052.

In some embodiments, the effector protein comprises a sequence selected from any one of SEQ ID NOs: 773, 775, and 793, and the guide RNA comprises (a) a repeat sequence comprising SEQ ID NO: 488 and (b) a spacer sequence selected from any one of SEQ ID NOs: 300-487, 822, and 1970-1995. In some embodiments, the system further comprises (c) an intermediary sequence comprising SEQ ID NO: 489 or (d) a handle sequence comprising SEQ ID NO: 490. In some embodiments, the guide RNA sequence comprises any one of SEQ ID NOs: 585-772, 829, and 2027-2052.

In some embodiments, the effector protein consists of a sequence selected from any one of SEQ ID NOs: 773, 775, or 793, and the guide RNA consists of (a) a repeat sequence consisting of SEQ ID NO: 488 and (b) a spacer sequence consisting of a sequence selected from any one of SEQ ID NOs: 300-487, 822, or 1970-1995. In some embodiments, the system further comprises (c) an intermediary sequence consisting of SEQ ID NO: 489 or (d) a handle sequence consisting of SEQ ID NO: 490. In some embodiments, the guide RNA sequence consists of a sequence selected from any one of SEQ ID NOs: 585-772, 829, or 2027-2052.

In some embodiments, the effector protein comprises an amino acid sequence that is at least 90%, at least, 91%, at least 92%, at least 93%, at least 94%, at least 95%, at least 96%, at least 97%, at least 98%, or at least 99% identical to a sequence selected from any one of SEQ ID NOs: 773, 775, and 793, wherein the effector protein is fused to a base editing enzyme, and the guide RNA comprises (a) a repeat sequence that is at least 90%, at least, 91%, at least 92%, at least 93%, at least 94%, at least 95%, at least 96%, at least 97%, at least 98%, or at least 99% identical to SEQ ID NO: 488, and (b) a spacer sequence that is at least 90%, at least, 91%, at least 92%, at least 93%, at least 94%, at least 95%, at least 96%, at least 97%, at least 98%, or at least 99% identical to a sequence selected from any one of SEQ ID NOs: 1970-1995. In some embodiments, the system further comprises (c) an intermediary sequence that is at least 90%, at least, 91%, at least 92%, at least 93%, at least 94%, at least 95%, at least 96%, at least 97%, at least 98%, or at least 99% identical to SEQ ID NO: 489 or (d) a handle sequence that is at least 90%, at least, 91%, at least 92%, at least 93%, at least 94%, at least 95%, at least 96%, at least 97%, at least 98%, or at least 99% identical to SEQ ID NO: 490. In some embodiments, the guide RNA sequence is at least 90%, at least, 91%, at least 92%, at least 93%, at least 94%, at least 95%, at least 96%, at least 97%, at least 98%, or at least 99% identical to a sequence selected from any one of SEQ ID NOs: 2027-2052.

In some embodiments, the effector protein comprises a sequence selected from any one of SEQ ID NOs: 773, 775, and 793, wherein the effector protein is fused to a base editing enzyme, and the guide RNA comprises (a) a repeat sequence comprising SEQ ID NO: 488 and (b) a spacer sequence selected from any one of SEQ ID NOs: 1970-1995. In some embodiments, the system further comprises (c) an intermediary sequence comprising SEQ ID NO: 489 or (d) a handle sequence comprising SEQ ID NO: 490. In some embodiments, the guide RNA sequence comprises any one of SEQ ID NOs: 2027-2052.

In some embodiments, the effector protein consists of a sequence selected from any one of SEQ ID NOs: 773, 775, or 793, wherein the effector protein is fused to a base editing enzyme, and the guide RNA consists of (a) a repeat sequence consisting of SEQ ID NO: 488 and (b) a spacer sequence consisting of a sequence selected from any one of SEQ ID NOs: 1970-1995. In some embodiments, the system further comprises (c) an intermediary sequence consisting of SEQ ID NO: 489 or (d) a handle sequence consisting of SEQ ID NO: 490. In some embodiments, the guide RNA sequence consists of a sequence selected from any one of SEQ ID NOs: 2027-2052.

### Exemplary ANGPTL3 Systems

In some embodiments, the present disclosure provides a system comprising (1) a guide RNA or a polynucleotide encoding the same, wherein the guide RNA comprises a spacer sequence that is capable of hybridizing to a target nucleic acid sequence in the ANGPTL3 gene; and (2) an effector protein or fusion protein thereof or a polynucleotide encoding the same.

In some embodiments, the effector protein comprises an amino acid sequence that is at least 90%, at least, 91%, at least 92%, at least 93%, at least 94%, at least 95%, at least 96%, at least 97%, at least 98%, or at least 99% identical to any one of the sequences recited in TABLEs 15, 18, and 19, and the guide RNA comprises (a) a repeat sequence that is at least 90%, at least, 91%, at least 92%, at least 93%, at least 94%, at least 95%, at least 96%, at least 97%, at least 98%, or at least 99% identical to a sequence selected from any one of SEQ ID NOs: 16 and 38-43 and (b) a spacer sequence that is at least 90%, at least, 91%, at least 92%, at least 93%, at least 94%, at least 95%, at least 96%, at least 97%, at least 98%, at least 99% identical to a sequence selected from any one of SEQ ID NOs: 806-808. In some embodiments, the guide RNA sequence is at least 90%, at least, 91%, at least 92%, at least 93%, at least 94%, at least 95%, at least 96%, at least 97%, at least 98%, or at least 99% identical to a sequence selected from any one of SEQ ID NOs: 817-819.

In some embodiments, the effector protein comprises any one of the sequences recited in TABLEs 15, 18, and 19, and the guide RNA comprises (a) a repeat sequence comprising a sequence selected from any one of SEQ ID NOs: 16 and 38-43 and (b) a spacer sequence selected from any one of SEQ ID NOs: 806-808. In some embodiments, the guide RNA sequence comprises any one of SEQ ID NOs: 817-819.

In some embodiments, the effector protein consists of any one of the sequences recited in TABLEs 15, 18, or 19, and the guide RNA consists of (a) a repeat sequence consisting of a sequence selected from any one of SEQ ID NOs: 16 or 38-43 and (b) a spacer sequence consisting of a sequence selected from any one of SEQ ID NOs: 806-808. In some embodiments, the guide RNA sequence consists of a sequence selected from any one of SEQ ID NOs: 817-819.

In some embodiments, the effector protein comprises an amino acid sequence that is at least 90%, at least, 91%, at least 92%, at least 93%, at least 94%, at least 95%, at least 96%, at least 97%, at least 98%, or at least 99% identical to a sequence selected from any one of SEQ ID NOs: 32, 34, 794, and 2090, and the guide RNA comprises (a) a repeat sequence that is at least 90%, at least, 91%, at least 92%, at least 93%, at least 94%, at least 95%, at least 96%, at least 97%, at least 98%, at least 99% identical to a sequence selected from any one of SEQ ID NOs: 16 and 38-43 and (b) a spacer sequence that is at least 90%, at least, 91%, at least 92%, at least 93%, at least 94%, at least 95%, at least 96%, at least 97%, at least 98%, at least 99% identical to a sequence selected from any one of SEQ ID NOs: 806-808. In some embodiments, the guide RNA sequence is at least 90%, at least, 91%, at least 92%, at least 93%, at least 94%, at least 95%, at least 96%, at least 97%, at least 98%, or at least 99% identical to a sequence selected from any one of SEQ ID NOs: 817-819.

In some embodiments, the effector protein comprises a sequence selected from any one of SEQ ID NOs: 32, 34, 794, and 2090, and the guide RNA comprises (a) a repeat sequence comprising a sequence selected from any one of SEQ ID NOs: 16 and 38-43 and (b) a spacer sequence selected from any one of SEQ ID NOs: 806-808. In some embodiments, the guide RNA sequence comprises any one of SEQ ID NOs: 817-819.

In some embodiments, the effector protein consists of a sequence selected from any one of SEQ ID NOs: 32, 34, 794, or 2090, and the guide RNA consists of (a) a repeat sequence consisting of a sequence selected from any one of SEQ ID NOs: 16 or 38-43 and (b) a spacer sequence consisting of a sequence selected from any one of SEQ ID NOs: 806-808. In some embodiments, the guide RNA sequence consists of a sequence selected from any one of SEQ ID NOs: 817-819.

In some embodiments, the effector protein comprises an amino acid sequence that is at least 90%, at least, 91%, at least 92%, at least 93%, at least 94%, at least 95%, at least 96%, at least 97%, at least 98%, or at least 99% identical to any one of the sequences recited in TABLEs 15, 16, and 17, and the guide RNA comprises (a) a repeat sequence that is at least 90%, at least, 91%, at least 92%, at least 93%, at least 94%, at least 95%, at least 96%, at least 97%, at least 98%, or at least 99% identical to SEQ ID NO: 488, and (b) a spacer sequence that is at least 90%, at least, 91%, at least 92%, at least 93%, at least 94%, at least 95%, at least 96%, at least 97%, at least 98%, or at least 99% identical to a sequence selected from any one of SEQ ID NOs: 1996-2017. In some embodiments, the system further comprises (c) an intermediary sequence that is at least 90%, at least, 91%, at least 92%, at least 93%, at least 94%, at least 95%, at least 96%, at least 97%, at least 98%, or at least 99% identical to SEQ ID NO: 489 and (d) a handle sequence that is at least 90%, at least, 91%, at least 92%, at least 93%, at least 94%, at least 95%, at least 96%, at least 97%, at least 98%, or at least 99% identical to SEQ ID NO: 490. In some embodiments, the guide RNA sequence is at least 90%, at least, 91%, at least 92%, at least 93%, at least 94%, at least 95%, at least 96%, at least 97%, at least 98%, or at least 99% identical to a sequence selected from any one of SEQ ID NOs: 2053-2074.

In some embodiments, the effector protein comprises any one of the sequences recited in TABLEs 15, 16, and 17, and the guide RNA comprises (a) a repeat sequence comprising SEQ ID NO: 488 and (b) a spacer sequence selected from any one of SEQ ID NOs: 1996-2017. In some embodiments, the system further comprises (c) an intermediary sequence comprising SEQ ID NO: 489 and (d) a handle sequence comprising SEQ ID NO: 490. In some embodiments, the guide RNA sequence comprises any one of SEQ ID NOs: 2053-2074.

In some embodiments, the effector protein consists of any one of the sequences recited in TABLEs 15, 16, or 17, and the guide RNA consists of (a) a repeat sequence consisting of SEQ ID NO: 488 and (b) a spacer sequence consisting of a sequence selected from any one of SEQ ID NOs: 1996-2017. In some embodiments, the system further comprises (c) an intermediary sequence consisting of SEQ ID NO: 489 or (d) a handle sequence consisting of SEQ ID NO: 490. In some embodiments, the guide RNA sequence consists of a sequence selected from any one of SEQ ID NOs: 2053-2074.

In some embodiments, the effector protein comprises an amino acid sequence that is at least 90%, at least, 91%, at least 92%, at least 93%, at least 94%, at least 95%, at least 96%, at least 97%, at least 98%, or at least 99% identical to a sequence selected from any one of SEQ ID NOs: 773, 775, and 793, and the guide RNA comprises (a) a repeat sequence that is at least 90%, at least, 91%, at least 92%, at least 93%, at least 94%, at least 95%, at least 96%, at least 97%, at least 98%, or at least 99% identical to SEQ ID NO: 488, and (b) a spacer sequence that is at least 90%, at least, 91%, at least 92%, at least 93%, at least 94%, at least 95%, at least 96%, at least 97%, at least 98%, or at least 99% identical to a sequence selected from any one of SEQ ID NOs: 1996-2017. In some embodiments, the system further comprises (c) an intermediary sequence that is at least 90%, at least, 91%, at least 92%, at least 93%, at least 94%, at least 95%, at least 96%, at least 97%, at least 98%, or at least 99% identical to SEQ ID NO: 489 or (d) a handle sequence that is at least 90%, at least, 91%, at least 92%, at least 93%, at least 94%, at least 95%, at least 96%, at least 97%, at least 98%, or at least 99% identical to SEQ ID NO: 490. In some embodiments, the guide RNA sequence is at least 90%, at least, 91%, at least 92%, at least 93%, at least 94%, at least 95%, at least 96%, at least 97%, at least 98%, or at least 99% identical to a sequence selected from any one of SEQ ID NOs: 2053-2074.

In some embodiments, the effector protein comprises a sequence selected from any one of SEQ ID NOs: 773, 775, and 793, and the guide RNA comprises (a) a repeat sequence comprising SEQ ID NO: 488 and (b) a spacer sequence selected from any one of SEQ ID NOs: 1996-2017. In some embodiments, the system further comprises (c) an intermediary sequence comprising SEQ ID NO: 489 or (d) a handle sequence comprising SEQ ID NO: 490. In some embodiments, the guide RNA sequence comprises any one of SEQ ID NOs: 2053-2074.

In some embodiments, the effector protein consists of a sequence selected from any one of SEQ ID NOs: 773, 775, or 793, and the guide RNA consists of (a) a repeat sequence consisting of SEQ ID NO: 488 and (b) a spacer sequence consisting of a sequence selected from any one of SEQ ID NOs: 1996-2017. In some embodiments, the system further comprises (c) an intermediary sequence consisting of SEQ ID NO: 489 or (d) a handle sequence consisting of SEQ ID NO: 490. In some embodiments, the guide RNA sequence consists of a sequence selected from any one of SEQ ID NOs: 2053-2074.

In some embodiments, the effector protein comprises an amino acid sequence that is at least 90%, at least, 91%, at least 92%, at least 93%, at least 94%, at least 95%, at least 96%, at least 97%, at least 98%, or at least 99% identical to a sequence selected from any one of SEQ ID NOs: 773, 775, and 793, wherein the effector protein is fused to a base editing enzyme, and the guide RNA comprises (a) a repeat sequence that is at least 90%, at least, 91%, at least 92%, at least 93%, at least 94%, at least 95%, at least 96%, at least 97%, at least 98%, or at least 99% identical to SEQ ID NO: 488, and (b) a spacer sequence that is at least 90%, at least, 91%, at least 92%, at least 93%, at least 94%, at least 95%, at least 96%, at least 97%, at least 98%, or at least 99% identical to a sequence selected from any one of SEQ ID NOs: 1996-2017. In some embodiments, the system further comprises (c) an intermediary sequence that is at least 90%, at least, 91%, at least 92%, at least 93%, at least 94%, at least 95%, at least 96%, at least 97%, at least 98%, or at least 99% identical to SEQ ID NO: 489 or (d) a handle sequence that is at least 90%, at least, 91%, at least 92%, at least 93%, at least 94%, at least 95%, at least 96%, at least 97%, at least 98%, or at least 99% identical to SEQ ID NO: 490. In some embodiments, the guide RNA sequence is at least 90%, at least, 91%, at least 92%, at least 93%, at least 94%, at least 95%, at least 96%, at least 97%, at least 98%, or at least 99% identical to a sequence selected from any one of SEQ ID NOs: 2053-2074.

In some embodiments, the effector protein comprises a sequence selected from any one of SEQ ID NOs: 773, 775, and 793, wherein the effector protein is fused to a base editing enzyme, and the guide RNA comprises (a) a repeat sequence comprising SEQ ID NO: 488 and (b) a spacer sequence selected from any one of SEQ ID NOs: 1996-2017. In some embodiments, the system further comprises (c) an intermediary sequence comprising SEQ ID NO: 489 or (d) a handle sequence comprising SEQ ID NO: 490. In some embodiments, the guide RNA sequence comprises any one of SEQ ID NOs: 2053-2074.

In some embodiments, the effector protein consists of a sequence selected from any one of SEQ ID NOs: 773, 775, or 793, wherein the effector protein is fused to a base editing enzyme, and the guide RNA consists of (a) a repeat sequence consisting of SEQ ID NO: 488 and (b) a spacer sequence consisting of a sequence selected from any one of SEQ ID NOs: 1996-2017. In some embodiments, the system further comprises (c) an intermediary sequence consisting of SEQ ID NO: 489 or (d) a handle sequence consisting of SEQ ID NO: 490. In some embodiments, the guide RNA sequence consisting of a sequence selected from any one of SEQ ID NOs: 2053-2074.

### 6. Target Nucleic Acids

Disclosed herein are compositions, systems, and methods for detecting and/or editing a target nucleic acid (e.g., the APOC3, the PCSK9, or the ANGPTL3 genes).

In some embodiments, the target nucleic acid is the APOC3 gene or a portion thereof. In some embodiments, the target nucleic acid is a gene that encodes the apolipoprotein C3 (APOC3) protein. In general, guide nucleic acids described herein comprise a sequence that is complementary to and/or hybridizes to a target sequence of the APOC3 gene. Exemplary reference sequence for the APOC3 gene are provided in TABLE 24. The target sequence of the APOC3 gene may be a portion of the APOC3 gene that encodes the APOC3 protein. Exemplary reference sequence for the APOC3 protein are listed in TABLE 25.

TABLE 24
Exemplary reference APOC3 genes

HGNC: 610; NCBI Entrez Gene: 354; Ensembl: ENSG00000110245; MIM: 107720;
UniProtKB/Swiss-Prot: P02656; RefSeq NM_000040.3; RefSeq NG_008949.1

TABLE 25
Exemplary reference APOC3 proteins

NCBI Reference Sequence: NP_000031.1;
Protein Accession: AJA40867.1; Protein Accession: AAB59372; GenBank: AAI34420.1

In some embodiments, the target nucleic acid is the PCSK9 gene or a portion thereof. In some embodiments, the target nucleic acid is a gene that encodes the Proprotein convertase subtilisin/kexin type 9 (PCSK9) protein. In general, guide nucleic acids described herein comprise a sequence that is complementary to and/or hybridizes to a target sequence of the PCSK9 gene. Exemplary reference sequence for the PCSK9 gene are provided in TABLE 26. The target sequence of the PCSK9 gene may be a portion of the PCSK9 gene that encodes the PCSK9 protein. Exemplary reference sequence for the PCSK9 protein are listed in TABLE 27.

TABLE 26
Exemplary reference PCSK9 genes

HGNC: 20001; NCBI Entrez Gene: 255738; Ensembl: ENSG00000169174; MIM: 607786;
UniProtKB/Swiss-Prot: Q8NBP7; RefSeq NM_174936.4; RefSeq NG_009061.1

TABLE 27
Exemplary reference PCSK9 proteins

NCBI Reference Sequence: NP_777596;
Protein Accession: Q8NBP7

In some embodiments, the target nucleic acid is the ANGPTL3 gene or a portion thereof. In some embodiments, the target nucleic acid is a gene that encodes the Angiopoietin-like 3 (ANGPTL3) protein. In general, guide nucleic acids described herein comprise a sequence that is complementary to and/or hybridizes to a target sequence of the ANGPTL3 gene. Exemplary reference sequence for the ANGPTL3 gene are provided in TABLE 28. The target sequence of the ANGPTL3 gene may be a portion of the ANGPTL3 gene that encodes the ANGPTL3 protein. Exemplary reference sequence for the ANGPTL3 protein are listed in TABLE 29.

TABLE 28
Exemplary reference ANGPTL3 genes

HGNC: 491; NCBI Entrez Gene: 27329; Ensembl: ENSG00000132855; MIM: 604774;
UniProtKB/Swiss-Prot: Q9Y5C1; RefSeq NM_ NM_014495; RefSeq NG_ NG_028169

TABLE 29
Exemplary reference ANGPTL3 proteins

NCBI Reference Sequence: NP_055310;
Protein Accession: Q9Y5C1

### Certain Samples

Systems, compositions, and methods described herein may be useful for detecting a mutated APOC3, PCSK9, or ANGPTL3 gene in a sample. In some embodiments, the sample is a biological sample, an environmental sample, or a combination thereof. Non-limiting examples of biological samples are blood, serum, plasma, saliva, urine, mucosal sample, peritoneal sample, cerebrospinal fluid, gastric secretions, nasal secretions, sputum, pharyngeal exudates, urethral or vaginal secretions, an exudate, an effusion, and a tissue sample (e.g., a biopsy sample). A tissue sample from a subject may be dissociated or liquified prior to application to detection system of the present disclosure. Non-limiting examples of environmental samples are soil, air, or water. In some embodiments, an environmental sample is taken as a swab from a surface of interest or taken directly from the surface of interest.

### 7. Vectors

Compositions, systems, and methods described herein comprise a vector or a use thereof. A vector can comprise a nucleic acid of interest (e.g., an APOC3-targeting guide nucleic acid, a PCSK9-targeting guide nucleic acid, an ANGPTL3-targeting guide nucleic acid, or polynucleotide encoding the same). In some embodiments, the nucleic acid of interest comprises one or more components of a composition or system described herein (e.g., an APOC3-targeting guide nucleic acid, a PCSK9-targeting guide nucleic acid, an ANGPTL3-targeting guide nucleic acid, or polynucleotide encoding the same). In some embodiments, the nucleic acid of interest comprises a nucleotide sequence that encodes one or more components of the composition or system described herein. In some embodiments, one or more components comprises a polypeptide(s), guide nucleic acid(s), target nucleic acid(s), and donor nucleic acid(s). In some embodiments, the component comprises a nucleic acid encoding an effector protein and a guide nucleic acid or a nucleic acid encoding the guide nucleic acid. The vector may be part of a vector system, wherein a vector system comprises a library of vectors each encoding one or more component of a composition or system described herein. In some embodiments, components described herein (e.g., an effector protein, a guide nucleic acid, and/or a target nucleic acid) are encoded by the same vector. In some embodiments, components described herein (e.g., an effector protein, a guide nucleic acid, and/or a target nucleic acid) are each encoded by different vectors of the system.

In some embodiments, a vector comprises a nucleotide sequence encoding one or more effector proteins as described herein. In some embodiments, the one or more effector proteins comprise at least two effector proteins. In some embodiments, the at least two effector protein are the same. In some embodiments, the at least two effector proteins are different from each other. In some embodiments, the nucleotide sequence is operably linked to a promoter that is operable in a target cell, such as a eukaryotic cell. In some embodiments, the vector comprises the nucleotide sequence encoding 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more effector proteins.

In some embodiments, a vector may encode one or more of any system components, including but not limited to effector proteins, guide nucleic acids, donor nucleic acids, and target nucleic acids as described herein. In some embodiments, a system component encoding sequence is operably linked to a promoter that is operable in a target cell, such as a eukaryotic cell. In some embodiments, a vector may encode 1, 2, 3, 4 or more of any system components. For example, a vector may encode two or more guide nucleic acids, wherein each guide nucleic acid comprises a different sequence. A vector may comprise the nucleic acid encoding an effector protein and a guide nucleic acid. A vector may encode an effector protein, a guide nucleic acid, and a donor nucleic acid.

In some embodiments, a vector comprises one or more guide nucleic acids, or a nucleotide sequence encoding the one or more guide nucleic acids as described herein (e.g., an APOC3-targeting guide nucleic acid, a PCSK9-targeting guide nucleic acid, an ANGPTL3-targeting guide nucleic acid, or polynucleotide encoding the same). In some embodiments, the one or more guide nucleic acids comprise at least two guide nucleic acids. In some embodiments, the at least two guide nucleic acids are the same. In some embodiments, the at least two guide nucleic acids are different from each other. In some embodiments, the guide nucleic acid or the nucleotide sequence encoding the guide nucleic acid is operably linked to a promoter that is operable in a target cell, such as a eukaryotic cell. In some embodiments, the vector comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more guide nucleic acids. In some embodiments, the vector comprises a nucleotide sequence encoding 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more guide nucleic acids.

In some embodiments, a vector may comprise or encode one or more regulatory elements. Regulatory elements may refer to transcriptional and translational control sequences, such as promoters, enhancers, polyadenylation signals, terminators, protein degradation signals, and the like, that provide for and/or regulate transcription of a non-coding sequence or a coding sequence and/or regulate translation of an encoded polypeptide. In some embodiments, a vector may comprise or encode for one or more additional elements, such as, for example, replication origins, antibiotic resistance (or a nucleic acid encoding the same), a tag (or a nucleic acid encoding the same), selectable markers, and the like. In some embodiments, a vector comprises or encodes for one or more elements, such as, for example, ribosome binding sites, and RNA splice sites.

Vectors described herein can encode a promoter—a regulatory region on a nucleic acid, such as a DNA sequence, capable of initiating transcription of a downstream (3′ direction) coding or non-coding sequence. A promoter can be linked at its 3′ terminus to a nucleic acid, the expression or transcription of which is desired, and extends upstream (5′ direction) to include bases or elements necessary to initiate transcription or induce expression, which could be measured at a detectable level. A promoter can comprise a nucleotide sequence, referred to herein as a “promoter sequence.” The promoter sequence can include a transcription initiation site, and one or more protein binding domains responsible for the binding of transcription machinery, such as RNA polymerase. When eukaryotic promoters are used, such promoters can contain “TATA” boxes and “CAT” boxes. Various promoters, including inducible promoters, may be used to drive expression, i.e., transcriptional activation, of the nucleic acid of interest. Accordingly, in some embodiments, the nucleic acid of interest can be operably linked to a promoter.

Promotors may be any suitable type of promoter envisioned for the compositions, systems, and methods described herein. Examples include constitutively active promoters (e.g., CMV promoter), inducible promoters (e.g., heat shock promoter, tetracycline-regulated promoter, steroid-regulated promoter, metal-regulated promoter, estrogen receptor-regulated promoter, etc.), spatially restricted and/or temporally restricted promoters (e.g., a tissue specific promoter, a cell type specific promoter, etc.), etc. Suitable promoters include, but are not limited to: SV40 early promoter, mouse mammary tumor virus long terminal repeat (LTR) promoter; adenovirus major late promoter (Ad MLP); a herpes simplex virus (HSV) promoter, a cytomegalovirus (CMV) promoter such as the CMV immediate early promoter region (CMVIE), a rous sarcoma virus (RSV) promoter, a human U6 small nuclear promoter (U6), an enhanced U6 promoter, and a human H1 promoter (H1). By transcriptional activation, it is intended that transcription will be increased above basal levels in the target cell by 2 fold, 5 fold, 10 fold, 50 fold, by 100 fold, 500 fold, or by 1000 fold, or more. In addition, vectors used for providing a nucleic acid that, when transcribed, produces a guide nucleic acid and/or a nucleic acid that encodes an effector protein to a cell may include nucleic acid sequences that encode for selectable markers in the target cells, so as to identify cells that have taken up the guide nucleic acid and/or the effector protein.

In general, vectors provided herein comprise at least one promotor or a combination of promoters driving expression or transcription of one or more genome editing tools described herein. In some embodiments, the vector comprises a nucleotide sequence of a promoter. In some embodiments, the vector comprises two promoters. In some embodiments, the vector comprises three promoters. In some embodiments, the length of the promoter is less than about 500, less than about 400, less than about 300, or less than about 200 linked nucleotides. In some embodiments, a length of the promoter is at least 100, at least 200, at least 300, at least 400, or at least 500 linked nucleotides. Non-limiting examples of promoters include CMV, 7SK, EF1a, RPBSA, hPGK, EFS, SV40, PGK1, Ubc, human beta actin promoter, CAG, TRE, UAS, Ac5, Polyhedrin, CaMKIIa, GAL1-10, H1, TEF1, GDS, ADH1, HSV TK, Ubi, U6, MNDU3, MSCV, MND and CAG. In some embodiments, the promoter allows for expression in a liver cell.

In some embodiments, the promoter is a constitutive promoter. In some embodiments, the promoter is an inducible promoter. In some embodiments, the inducible promoter only drives expression of its corresponding coding sequence (e.g., polypeptide or guide nucleic acid) when a signal is present, e.g., a hormone, a small molecule, a peptide. Non-limiting examples of inducible promoters are the T7 RNA polymerase promoter, the T3 RNA polymerase promoter, the Isopropyl-beta-D-thiogalactopyranoside (IPTG)-regulated promoter, a lactose induced promoter, a heat shock promoter, a tetracycline-regulated promoter (tetracycline-inducible or tetracycline-repressible), a steroid regulated promoter, a metal-regulated promoter, and an estrogen receptor-regulated promoter. In some embodiments, the promoter is an activation-inducible promoter, such as a CD69 promoter. In some embodiments, the promoter for expressing effector protein is a muscle-specific promoter. In some embodiments, the muscle-specific promoter comprises Ck8e, SPC5-12, Mb, or Desmin promoter sequence. In some embodiments, the promoter for expressing effector protein is a ubiquitous promoter. In some embodiments, the ubiquitous promoter comprises MND or CAG promoter sequence.

In some embodiments, the promoters are prokaryotic promoters (e.g., drive expression of a gene in a prokaryotic cell). In some embodiments, the promoters are eukaryotic promoters, (e.g., drive expression of a gene in a eukaryotic cell). In some embodiments, the promoter is EF1a. In some embodiments, the promoter is ubiquitin. In some embodiments, vectors are bicistronic or polycistronic vector (e.g., having or involving two or more loci responsible for generating a protein) having an internal ribosome entry site (IRES) is for translation initiation in a cap-independent manner.

In some embodiments, a vector described herein is a nucleic acid expression vector. In some embodiments, a vector described herein is a recombinant expression vector. In some embodiments, a vector described herein is a messenger RNA.

In some embodiments, the expression vector comprises the DNA molecule encoding a guide nucleic acid. In some embodiments, the expression vector further comprises the nucleic acid encoding an effector protein. In some embodiments, the expression vector further comprises or encodes a donor nucleic acid. In some embodiments, the expression vector encoding a guide nucleic acid, wherein the guide nucleic acid comprises a first region comprising a repeat; and a second region comprising a spacer sequence that is complementary to a target sequence of an APOC3 gene. In some embodiments, wherein the first region is located 5′ of the second region. In some embodiments, the expression vector further comprises an effector protein that binds the repeat sequence or a nucleic acid encoding the effector protein. In some embodiments, the spacer comprises a nucleotide sequence that is at least 70%, at least 75%, at least 80%, at least 85%, at least 90%, at least 95%, or 100% identical to a sequence selected from SEQ ID NOs: 1-15, 67-72, 207, 804-805, and 830-999; the repeat sequence comprises a nucleotide sequence that is at least 70%, at least 75%, at least 80%, at least 85%, at least 90%, at least 95%, or 100% identical to a sequence selected from SEQ ID NOs: 16, and 38-43; the effector protein comprises an amino acid sequence that is at least 65%, at least 70%, at least 75%, at least 80%, at least 85%, at least 90%, at least 95%, at least 97%, at least 98%, at least 99%, or 100% identical to any sequence listed in TABLES 15, 18, and 19; or a combination thereof. In some embodiments, the spacer comprises a nucleotide sequence that is at least 70%, at least 75%, at least 80%, at least 85%, at least 90%, at least 95%, or 100% identical to a sequence selected from SEQ ID NOs: 209-299, 823-825, 1000-1399, 2018-2026, and 2084-2086; the repeat sequence comprises a nucleotide sequence that is at least 70%, at least 75%, at least 80%, at least 85%, at least 90%, at least 95%, or 100% identical to SEQ ID NO: 488; the effector protein comprises an amino acid sequence that is at least 65%, at least 70%, at least 75%, at least 80%, at least 85%, at least 90%, at least 95%, at least 97%, at least 98%, at least 99%, or 100% identical to any sequence listed in TABLES 15-17; or a combination thereof.

In some embodiments, the expression vector encoding a guide nucleic acid, wherein the guide nucleic acid comprises a first region comprising a repeat; and a second region comprising a spacer sequence that is complementary to a target sequence of a PCSK9 gene. In some embodiments, wherein the first region is located 5′ of the second region. In some embodiments, the expression vector further comprises an effector protein that binds the repeat sequence or a nucleic acid encoding the effector protein. In some embodiments, the spacer comprises a nucleotide sequence that is at least 70%, at least 75%, at least 80%, at least 85%, at least 90%, at least 95%, or 100% identical to a sequence selected from SEQ ID NOs: 79-140, 208, 799-803, and 809; the repeat sequence comprises a nucleotide sequence that is at least 70%, at least 75%, at least 80%, at least 85%, at least 90%, at least 95%, or 100% identical to a sequence selected from SEQ ID NOs: 16, and 38-43; the effector protein comprises an amino acid sequence that is at least 65%, at least 70%, at least 75%, at least 80%, at least 85%, at least 90%, at least 95%, at least 97%, at least 98%, at least 99%, or 100% identical to any sequence listed in TABLES 15, 18, and 19; or a combination thereof. In some embodiments, the spacer comprises a nucleotide sequence that is at least 70%, at least 75%, at least 80%, at least 85%, at least 90%, at least 95%, or 100% identical to a sequence selected from SEQ ID NOs: 300-487, 822 and 1970-1995; the repeat sequence comprises a nucleotide sequence that is at least 70%, at least 75%, at least 80%, at least 85%, at least 90%, at least 95%, or 100% identical to SEQ ID NO: 488; the effector protein comprises an amino acid sequence that is at least 65%, at least 70%, at least 75%, at least 80%, at least 85%, at least 90%, at least 95%, at least 97%, at least 98%, at least 99%, or 100% identical to any sequence listed in TABLES 15-17; or a combination thereof.

In some embodiments, the expression vector encoding a guide nucleic acid, wherein the guide nucleic acid comprises a first region comprising a repeat; and a second region comprising a spacer sequence that is complementary to a target sequence of a ANGPTL3 gene. In some embodiments, wherein the first region is located 5′ of the second region. In some embodiments, the expression vector further comprises an effector protein that binds the repeat sequence or a nucleic acid encoding the effector protein. In some embodiments, the spacer comprises a nucleotide sequence that is at least 70%, at least 75%, at least 80%, at least 85%, at least 90%, at least 95%, or 100% identical to a sequence selected from SEQ ID NOs: 806-808; the repeat sequence comprises a nucleotide sequence that is at least 70%, at least 75%, at least 80%, at least 85%, at least 90%, at least 95%, or 100% identical to a sequence selected from SEQ ID NOs: 16, and 38-43; the effector protein comprises an amino acid sequence that is at least 65%, at least 70%, at least 75%, at least 80%, at least 85%, at least 90%, at least 95%, at least 97%, at least 98%, at least 99%, or 100% identical to any sequence listed in TABLES 15, 18, and 19; or a combination thereof. In some embodiments, the spacer comprises a nucleotide sequence that is at least 70%, at least 75%, at least 80%, at least 85%, at least 90%, at least 95%, or 100% identical to a sequence selected from SEQ ID NOs: 1996-2017; the repeat sequence comprises a nucleotide sequence that is at least 70%, at least 75%, at least 80%, at least 85%, at least 90%, at least 95%, or 100% identical to SEQ ID NO: 488; the effector protein comprises an amino acid sequence that is at least 65%, at least 70%, at least 75%, at least 80%, at least 85%, at least 90%, at least 95%, at least 97%, at least 98%, at least 99%, or 100% identical to any sequence listed in TABLES 15-17; or a combination thereof.

In some embodiments, a vector described herein is a delivery vector. In some embodiments, the delivery vector is a eukaryotic vector, a prokaryotic vector (e.g., a bacterial vector) a viral vector, or any combination thereof. In some embodiments, the delivery vehicle is a non-viral vector. In some embodiments, the delivery vector is a plasmid. In some embodiments, the plasmid comprises DNA. In some embodiments, the plasmid comprises RNA. In some embodiments, the plasmid comprises circular double-stranded DNA. In some embodiments, the plasmid is linear. In some embodiments, the plasmid comprises one or more coding sequences of interest and one or more regulatory elements. In some embodiments, the plasmid comprises a bacterial backbone containing an origin of replication and an antibiotic resistance gene or other selectable marker for plasmid amplification in bacteria. In some embodiments, the plasmid is a minicircle plasmid. In some embodiments, the plasmid contains one or more genes that provide a selective marker to induce a target cell to retain the plasmid. In some examples, the plasmids are engineered through synthetic or other suitable means known in the art. For example, in some embodiments, the genetic elements are assembled by restriction digest of the desired genetic sequence from a donor plasmid or organism to produce ends of the DNA which is then be readily ligated to another genetic sequence.

In some embodiments, vectors comprise an enhancer. Enhancers are nucleotide sequences that have the effect of enhancing promoter activity. In some embodiments, enhancers augment transcription regardless of the orientation of their sequence. In some embodiments, enhancers activate transcription from a distance of several kilo basepairs. Furthermore, enhancers are located optionally upstream or downstream of a gene region to be transcribed, and/or located within the gene, to activate the transcription. Exemplary enhancers include, but are not limited to, WPRE; CMV enhancers; the R-U5′ segment in LTR of HTLV-I.

In some embodiments, disclosed herein comprise one or more nucleic acids encoding an effector protein, fusion effector protein, fusion partner, a guide nucleic acid, or a combination thereof. The effector protein, fusion effector protein, fusion partner protein, or combination thereof may be any one of those described herein. In some embodiments, of the above, the nucleic acid expression vector comprises a polynucleotide encoding an effector protein that is at least 80%, at least 85%, at least 90%, at least 95%, at least 99%, or 100% identical to any one of the sequences recited in TABLES 15-19.

The one or more nucleic acids may comprise a plasmid. The one or more nucleic acids may comprise a nucleic acid expression vector. The one or more nucleic acids may comprise a viral vector. In some embodiments, the viral vector is a lentiviral vector. In some embodiments, the vector is an adeno-associated viral (AAV) vector. In some embodiments, compositions, including pharmaceutical compositions, comprise a viral vector encoding a fusion effector protein and a guide nucleic acid, wherein at least a portion of the guide nucleic acid binds to the effector protein of the fusion effector protein. In some embodiments, pharmaceutical compositions comprise one or more nucleic acids encoding an effector protein, fusion effector protein, fusion partner, a guide nucleic acid, or a combination thereof; and a pharmaceutically acceptable carrier or diluent.

### Administration of a Non-Viral Vector

In some embodiments, an administration of a non-viral vector comprises contacting a cell, such as a host cell, with the non-viral vector. In some embodiments, a physical method or a chemical method is employed for delivering the vector into the cell. Exemplary physical methods include electroporation, gene gun, sonoporation, magnetofection, or hydrodynamic delivery. Exemplary chemical methods include delivery of the recombinant polynucleotide by liposomes such as, cationic lipids or neutral lipids; lipofection; dendrimers; lipid nanoparticle (LNP); or cell-penetrating peptides.

In some embodiments, a vector is administered as part of a method of nucleic acid detection, editing, and/or treatment as described herein. In some embodiments, a vector is administered in a single vehicle, such as a single expression vector. In some embodiments, at least two of the three components, a nucleic acid encoding one or more effector proteins, one or more donor nucleic acids, and one or more guide nucleic acids or a nucleic acid encoding the one or more guide nucleic acid, are provided in the single expression vector. In some embodiments, components, such as a guide nucleic acid and an effector protein, are encoded by the same vector.

In some embodiments, an effector protein (or a nucleic acid encoding same) and/or an engineered guide nucleic acid (or a nucleic acid that, when transcribed, produces same) are not co-administered with donor nucleic acid in a single vehicle. In some embodiments, an effector protein (or a nucleic acid encoding same), an engineered guide nucleic acid (or a nucleic acid that, when transcribed, produces same), and/or donor nucleic acid are administered in one or more or two or more vehicles, such as one or more, or two or more expression vectors.

In some embodiments, a vector may be part of a vector system. In some embodiments, the vector system comprises a library of vectors each encoding one or more components of a composition or system described herein. In some embodiments, a vector system is administered as part of a method of nucleic acid detection, editing, and/or treatment as described herein, wherein at least two vectors are co-administered. In some embodiments, the at least two vectors comprise different components. In some embodiments, the at least two vectors comprise the same component having different sequences. In some embodiments, at least one of the three components, a nucleic acid encoding one or more effector proteins, one or more donor nucleic acids, and one or more guide nucleic acids or a nucleic acid encoding the one or more guide nucleic acids, or a variant thereof is provided in a different vector. In some embodiments, the nucleic acid encoding the effector protein, and a guide nucleic acid or a nucleic acid encoding the guide nucleic acid are provided in different vectors. In some embodiments, the donor nucleic acid is encoded by a different vector than the vector encoding the effector protein and the guide nucleic acid.

### Lipid Particles and Non-Viral Vectors

In some embodiments, compositions and systems provided herein comprise a lipid particle. In some embodiments, a lipid particle is a lipid nanoparticle (LNP). In some embodiments, a lipid or a lipid nanoparticle can encapsulate an expression vector as described herein. LNPs are a non-viral delivery system for delivery of the composition and/or system components described herein. LNPs are particularly effective for delivery of nucleic acids. Beneficial properties of LNP include ease of manufacture, low cytotoxicity and immunogenicity, high efficiency of nucleic acid encapsulation and cell transfection, multi-dosing capabilities and flexibility of design (Kulkarni et al., (2018) Nucleic Acid Therapeutics, 28(3):146-157). In some embodiments, compositions and methods comprise a lipid, polymer, nanoparticle, or a combination thereof, or use thereof, to introduce one or more effector proteins, one or more guide nucleic acids, one or more donor nucleic acids, or any combinations thereof to a cell. Non-limiting examples of lipids and polymers are cationic polymers, cationic lipids, ionizable lipids, or bio-responsive polymers. In some embodiments, the ionizable lipids exploits chemical-physical properties of the endosomal environment (e.g., pH) offering improved delivery of nucleic acids. In some embodiments, the ionizable lipids are neutral at physiological pH. In some embodiments, the ionizable lipids are protonated under acidic pH. In some embodiments, the bio-responsive polymer exploits chemical-physical properties of the endosomal environment (e.g., pH) to preferentially release the genetic material in the intracellular space.

In some embodiments, a LNP comprises an outer shell and an inner core. In some embodiments, the outer shell comprises lipids. In some embodiments, the lipids comprise modified lipids. In some embodiments, the modified lipids comprise pegylated lipids. In some embodiments, the lipids comprise one or more of cationic lipids, anionic lipids, ionizable lipids, and non-ionic lipids. In some embodiments, the LNP comprises one or more of N1,N3,N5-tris(3-(didodecylamino)propyl)benzene-1,3,5-tricarboxamide (TT3), 2-dioleoyl-sn-glycero-3-phosphoethanolamine (DOPE), 1-palmitoyl-2-oleoylsn-glycero-3-phosphoethanolamine (POPE), 1,2-distearoyl-sn-glycero-3-phosphocholine (DSPC), cholesterol (Chol), 1,2-dimyristoyl-sn-glycerol, and methoxypolyethylene glycol (DMG-PEG), derivatives, analogs, or variants thereof. In some embodiments, the LNP has a negative net overall charge prior to complexation with one or more of a guide nucleic acid, a nucleic acid encoding the one or more guide nucleic acid, a nucleic acid encoding the effector protein, and/or a donor nucleic acid. In some embodiments, the inner core is a hydrophobic core. In some embodiments, the one or more of a guide nucleic acid, the nucleic acid encoding the one or more guide nucleic acid, the nucleic acid encoding the effector protein, and/or the donor nucleic acid forms a complex with one or more of the cationic lipids and the ionizable lipids. In some embodiments, the nucleic acid encoding the effector protein or the nucleic acid encoding the guide nucleic acid is self-replicating.

In some embodiments, a LNP comprises one or more of cationic lipids, ionizable lipids, and modified versions thereof. In some embodiments, the ionizable lipid comprises TT3 or a derivative thereof. Accordingly, in some embodiments, the LNP comprises one or more of TT3 and pegylated TT3. The publication WO2016187531 is hereby incorporated by reference in its entirety, which describes representative LNP formulations in Table 2 and Table 3, and representative methods of delivering LNP formulations in Example 7.

In some embodiments, a LNP comprises a lipid composition targeting to a specific organ. In some embodiments, the lipid composition comprises lipids having a specific alkyl chain length that controls accumulation of the LNP in the specific organ (e.g., liver or spleen). In some embodiments, the lipid composition comprises a biomimetic lipid that controls accumulation of the LNP in the specific organ (e.g., brain). In some embodiments, the lipid composition comprises lipid derivatives (e.g., cholesterol derivatives) that controls accumulation of the LNP in a specific cell (e.g., liver endothelial cells, Kupffer cells, hepatocytes).

### Delivery of Viral Vectors

In some embodiments, a vector described herein comprises a viral vector. In some embodiments, the viral vector comprises a nucleic acid to be delivered into a host cell by a recombinantly produced virus or viral particle. The nucleic acid may be single-stranded or double stranded, linear or circular, segmented or non-segmented. The nucleic acid may comprise DNA, RNA, or a combination thereof. In some embodiments, the vector is an adeno-associated viral vector. There are a variety of viral vectors that are associated with various types of viruses, including but not limited to retroviruses (e.g., lentiviruses and γ-retroviruses), adenoviruses, arenaviruses, alphaviruses, adeno-associated viruses (AAVs), baculoviruses, vaccinia viruses, herpes simplex viruses and poxviruses. In some embodiments, the vector is an adeno-associated viral (AAV) vector. In some embodiments, the viral vector is a recombinant viral vector. In some embodiments, the vector is a retroviral vector. In some embodiments, the retroviral vector is a lentiviral vector. In some embodiments, the retroviral vector comprises gamma-retroviral vector. A viral vector provided herein may be derived from or based on any such virus. For example, in some embodiments, the gamma-retroviral vector is derived from a Moloney Murine Leukemia Virus (MoMLV, MMLV, MuLV, or MLV) or a Murine Stem cell Virus (MSCV) genome. In some embodiments, the lentiviral vector is derived from the human immunodeficiency virus (HIV) genome. In some embodiments, the viral vector is a chimeric viral vector. In some embodiments, the chimeric viral vector comprises viral portions from two or more viruses. In some embodiments, the viral vector corresponds to a virus of a specific serotype.

In some embodiments, a viral vector is an adeno-associated viral vector (AAV vector). In some embodiments, a viral particle that delivers a viral vector described herein is an AAV. In some embodiments, the AAV comprises any AAV known in the art. In some embodiments, the viral vector corresponds to a virus of a specific AAV serotype. In some embodiments, the AAV serotype is selected from an AAV1 serotype, an AAV2 serotype, AAV3 serotype, an AAV4 serotype, AAV5 serotype, an AAV6 serotype, AAV7 serotype, an AAV8 serotype, an AAV9 serotype, an AAV10 serotype, an AAV11 serotype, an AAV12 serotype, an AAV-rh10 serotype, and any combination, derivative, or variant thereof. In some embodiments, the AAV vector is a recombinant vector, a hybrid AAV vector, a chimeric AAV vector, a self-complementary AAV (scAAV) vector, a single-stranded AAV, or any combination thereof. scAAV genomes are generally known in the art and contain both DNA strands which can anneal together to form double-stranded DNA.

In some embodiments, an AAV vector described herein is a chimeric AAV vector. In some embodiments, the chimeric AAV vector comprises an exogenous amino acid or an amino acid substitution, or capsid proteins from two or more serotypes. In some examples, a chimeric AAV vector may be genetically engineered to increase transduction efficiency, selectivity, or a combination thereof.

In some embodiments, AAV vector described herein comprises two inverted terminal repeats (ITRs). According, in some embodiments, the viral vector provided herein comprises two inverted terminal repeats of AAV. A nucleotide sequence between the ITRs of an AAV vector provided herein comprises a sequence encoding genome editing tools. In some embodiments, the genome editing tools comprise a nucleic acid encoding one or more effector proteins, a nucleic acid encoding one or more fusion proteins (e.g., a nuclear localization signal (NLS), polyA tail), one or more guide nucleic acids, a nucleic acid encoding the one or more guide nucleic acids, respective promoter(s), one or more donor nucleic acid, or any combinations thereof. In some embodiments, viral vectors provided herein comprise at least one promotor or a combination of promoters driving expression or transcription of one or more genome editing tools described herein. In some embodiments, a coding region of the AAV vector forms an intramolecular double-stranded DNA template thereby generating the AAV vector that is a self-complementary AAV (scAAV) vector. In some embodiments, the scAAV vector comprises the sequence encoding genome editing tools that has a length of about 2 kb to about 3 kb. In some embodiments, the AAV vector provided herein is a self-inactivating AAV vector. In some embodiments, the AAV vector provided herein comprises a modification, such as an insertion, deletion, chemical alteration, or synthetic modification, relative to a wild-type AAV vector.

### Producing AAV Delivery Vectors

In some embodiments, methods of producing AAV delivery vectors herein comprise packaging a nucleic acid encoding an effector protein and a guide nucleic acid, or a combination thereof, into an AAV vector. In some embodiments, methods of producing the delivery vector comprises, (a) contacting a cell with at least one nucleic acid encoding: (i) a guide nucleic acid; (ii) a Replication (Rep) gene; and (iii) a Capsid (Cap) gene that encodes an AAV capsid protein; (b) expressing the AAV capsid protein in the cell; (c) assembling an AAV particle; and (d) packaging an effector encoding nucleic acid into the AAV particle, thereby generating an AAV delivery vector. In some embodiments, promoters, stuffer sequences, and any combination thereof may be packaged in the AAV vector. In some examples, the AAV vector may package 1, 2, 3, 4, or 5 guide nucleic acids or copies thereof. In some embodiments, the AAV vector comprises inverted terminal repeats, e.g., a 5′ inverted terminal repeat and a 3′ inverted terminal repeat. In some embodiments, the AAV vector comprises a mutated inverted terminal repeat that lacks a terminal resolution site.

In some embodiments, a hybrid AAV vector is produced by transcapsidation, e.g., packaging an inverted terminal repeat (ITR) from a first serotype into a capsid of a second serotype, wherein the first and second serotypes may be not the same. In some examples, the Rep gene and ITR from a first AAV serotype (e.g., AAV2) may be used in a capsid from a second AAV serotype (e.g., AAV9), wherein the first and second AAV serotypes may be not the same. As a non-limiting example, a hybrid AAV serotype comprising the AAV2 ITRs and AAV9 capsid protein may be indicated AAV2/9. In some examples, the hybrid AAV delivery vector comprises an AAV2/1, AAV2/2, AAV 2/4, AAV2/5, AAV2/8, or AAV2/9 vector.

In some embodiments, the AAV vector comprises a recombinant AAV expression cassette comprising sequences encoding: a) a first inverted terminal repeat (ITR) and a first promoter; b) an effector protein disclosed herein; c) optionally a second promoter; d) a second polynucleotide encoding a guide nucleic acid disclosed here; and e) a second ITR. In some embodiments, the AAV expression cassette is a self-complementary AAV vector.

### Producing AAV Particles

In some embodiments, AAV particles described herein are recombinant AAV (rAAV). In some embodiments, rAAV particles are generated by transfecting AAV producing cells with an AAV-containing plasmid carrying the sequence encoding the genome editing tools, a plasmid that carries viral encoding regions, i.e., Rep and Cap gene regions; and a plasmid that provides the helper genes such as E1A, E1B, E2A, E40RF6 and VA. In some embodiments, the AAV producing cells are mammalian cells. In some embodiments, host cells for rAAV viral particle production are mammalian cells. In some embodiments, a mammalian cell for rAAV viral particle production is a COS cell, a HEK293T cell, a HeLa cell, a KB cell, a derivative thereof, or a combination thereof. In some embodiments, rAAV virus particles can be produced in the mammalian cell culture system by providing the rAAV plasmid to the mammalian cell. In some embodiments, producing rAAV virus particles in a mammalian cell can comprise transfecting vectors that express the rep protein, the capsid protein, and the gene-of-interest expression construct flanked by the ITR sequence on the 5′ and 3′ ends. Methods of such processes are provided in, for example, Naso et al., BioDrugs, 2017 August; 31(4):317-334 and Benskey et al., (2019), Methods Mol Biol., 1937:3-26, each of which is incorporated by reference in their entireties.

In some embodiments, rAAV is produced in a non-mammalian cell. In some embodiments, rAAV is produced in an insect cell. In some embodiments, the insect cell for producing rAAV viral particles comprises a Sf9 cell. In some embodiments, production of rAAV virus particles in insect cells can comprise baculovirus. In some embodiments, production of rAAV virus particles in insect cells can comprise infecting the insect cells with three recombinant baculoviruses, one carrying the cap gene, one carrying the rep gene, and one carrying the gene-of-interest expression construct enclosed by an ITR on both the 5′ and 3′ end. In some embodiments, rAAV virus particles are produced by the One Bac system. In some embodiments, rAAV virus particles can be produced by the Two Bac system. In some embodiments, in the Two Bac system, the rep gene and the cap gene of the AAV is integrated into one baculovirus virus genome, and the ITR sequence and the gene-of-interest expression construct is integrated into another baculovirus virus genome. In some embodiments, in the One Bac system, an insect cell line that expresses both the rep protein and the capsid protein is established and infected with a baculovirus virus integrated with the ITR sequence and the gene-of-interest expression construct. Details of such processes are provided in, for example, Smith et. al., (1983), Mol. Cell. Biol., 3(12):2156-65; Urabe et al., (2002), Hum. Gene. Ther., 1; 13(16):1935-43; and Benskey et al., (2019), Methods Mol Biol., 1937:3-26, each of which is incorporated by reference in its entirety.

### 8. Pharmaceutical Compositions and Modes of Administration

Disclosed herein are compositions comprising one or more effector proteins described herein or nucleic acids encoding the one or more effector proteins, one or more guide nucleic acids described herein or nucleic acids encoding the one or more guide nucleic acids described herein (e.g., APOC3-targeting guide nucleic acids, a PCSK9-targeting guide nucleic acid, a ANGPTL3-targeting guide nucleic acid, or polynucleotides encoding the same), or combinations thereof. In some embodiments, a repeat sequence of the one or more guide nucleic acids are capable of interacting with the one or more of the effector proteins. In some embodiments, spacer sequences of the one or more guide nucleic acids hybridizes with a target sequence of a target nucleic acid. In some embodiments, the compositions are capable of editing a target nucleic acid in a cell or a subject. In some embodiments, the compositions are capable of editing a target nucleic acid or the expression thereof in a cell, in a tissue, in an organ, in vitro, in vivo, or ex vivo. In some embodiments, the compositions are capable of editing a target nucleic acid in a sample comprising the target nucleic.

In some embodiments, compositions described herein comprise plasmids described herein, viral vectors described herein, non-viral vectors described herein, or combinations thereof. In some embodiments, compositions described herein comprise the viral vectors. In some embodiments, compositions described herein comprise an AAV. In some embodiments, compositions described herein comprise liposomes (e.g., cationic lipids or neutral lipids), dendrimers, lipid nanoparticle (LNP), or cell-penetrating peptides. In some embodiments, compositions described herein comprise an LNP.

In some embodiments, compositions described herein are pharmaceutical compositions. In some embodiments, the pharmaceutical compositions comprise compositions described herein and a pharmaceutically acceptable carrier or diluent. Non-limiting examples of pharmaceutically acceptable carriers and diluents suitable for the pharmaceutical compositions disclosed herein include buffers (e.g., neutral buffered saline, phosphate buffered saline); carbohydrates (e.g., glucose, mannose, sucrose, dextran, mannitol); polypeptides or amino acids (e.g., glycine); antioxidants; chelating agents (e.g., EDTA, glutathione); adjuvants (e.g., aluminum hydroxide); surfactants (Polysorbate 80, Polysorbate 20, or Pluronic F68); glycerol; sorbitol; mannitol; polyethylene glycol; and preservatives. In some embodiments, the vector is formulated for delivery through injection by a needle carrying syringe. In some embodiments, the composition is formulated for delivery by electroporation. In some embodiments, the composition is formulated for delivery by chemical method. In some embodiments, the pharmaceutical compositions comprise a virus vector or a non-viral vector.

Pharmaceutical compositions described herein comprise a salt. In some embodiments, the salt is a sodium salt. In some embodiments, the salt is a potassium salt. In some embodiments, the salt is a magnesium salt. In some embodiments, the salt is NaCl. In some embodiments, the salt is KNO₃. In some embodiments, the salt is Mg²⁺SO₄²⁻.

Pharmaceutical compositions described herein are in the form of a solution (e.g., a liquid). In some embodiments, the solution is formulated for injection, e.g., intravenous or subcutaneous injection. In some embodiments, the pH of the solution is about 7, about 7.1, about 7.2, about 7.3, about 7.4, about 7.5, about 7.6, about 7.7, about 7.8, about 7.9, about 8, about 8.1, about 8.2, about 8.3, about 8.4, about 8.5, about 8.6, about 8.7, about 8.8, about 8.9, or about 9. In some embodiments, the pH is 7 to 7.5, 7.5 to 8, 8 to 8.5, 8.5 to 9, or 7 to 8.5. In some cases, the pH of the solution is less than 7. In some cases, the pH is greater than 7.

Disclosed herein, in some embodiments, are pharmaceutical compositions for modifying a target nucleic acid in a cell or a subject, comprising any one of the effector proteins, engineered effector proteins, fusion effector proteins, or guide nucleic acids as described herein and any combination thereof. Also disclosed herein, are pharmaceutical compositions comprising a nucleic acid encoding any one of the effector proteins, engineered effector proteins, fusion effector proteins, or guide nucleic acids as described herein and any combination thereof. Also disclosed herein, are pharmaceutical compositions comprising the nucleic acid expression vector, the cell, or the population of cells disclosed herein. In some embodiments, pharmaceutical compositions comprise a plurality of guide nucleic acids. In some embodiments, the pharmaceutical composition disclosed herein also comprise a pharmaceutical acceptable carrier. Pharmaceutical compositions may be used to modify a target nucleic acid or the expression thereof in a cell in vitro, in vivo, or ex vivo. In some embodiments, pharmaceutical compositions comprise one or more nucleic acids encoding an effector protein, fusion effector protein, fusion partner, a guide nucleic acid, or a combination thereof; and a pharmaceutically acceptable carrier or diluent. The effector protein, fusion effector protein, fusion partner protein, or combination thereof may be any one of those described herein.

### 9. Methods of Detecting a Target Nucleic Acid

Provided herein are methods of detecting target nucleic acids. Methods may comprise detecting target nucleic acids with compositions or systems described herein. Methods may comprise detecting a target nucleic acid in a sample, e.g., a cell lysate, a biological fluid, or environmental sample. Methods may comprise detecting a target nucleic acid in a cell. In some embodiments, methods of detecting a target nucleic acid in a sample or cell comprises contacting the sample or cell with an effector protein or a multimeric complex thereof, a guide nucleic acid, wherein at least a portion of the guide nucleic acid is complementary to at least a portion of the target nucleic acid, and a reporter nucleic acid that is cleaved in the presence of the effector protein, the guide nucleic acid, and the target nucleic acid, and detecting a signal produced by cleavage of the reporter nucleic acid, thereby detecting the target nucleic acid in the sample. In some embodiments, methods result in trans cleavage of the reporter nucleic acid. In some embodiments, methods result in cis cleavage of the reporter nucleic acid.

### 10. Methods of Nucleic Acid Modification

Provided herein are methods of editing and/or modifying a target nucleic acid (e.g., a target nucleic acid in the APOC3, PCSK9, or ANGPTL3 genes). In general, editing refers to modifying the nucleobase sequence of a target nucleic acid. However, compositions and systems disclosed herein may also be capable of making epigenetic modifications of target nucleic acids. Effector proteins, multimeric complexes thereof and systems described herein may be used for editing or modifying a target nucleic acid. Editing a target nucleic acid may comprise one or more of: cleaving the target nucleic acid, deleting one or more nucleotides of the target nucleic acid, inserting one or more nucleotides into the target nucleic acid, mutating one or more nucleotides of the target nucleic acid, or modifying (e.g., methylating, demethylating, deaminating, or oxidizing) of one or more nucleotides of the target nucleic acid.

Methods of editing may comprise contacting a target nucleic acid with an effector protein described herein and a guide nucleic acid, wherein the effector protein comprises an amino acid sequence that is at least 70%, at least 75%, at least 80%, at least 85%, at least 90%, at least 95%, at least 97%, or at least 98%, at least 99%, or 100% identical to any one of the sequences set forth in TABLES 15, 18, and 19. In some embodiments, the effector protein comprises an amino acid sequence that is at least 70%, at least 75%, at least 80%, at least 85%, at least 90%, at least 95%, at least 97%, at least 98%, at least 99%, or 100% identical to any one of the sequences set forth in TABLES 15, 18, and 19, wherein the amino acid residue at position 26, relative to SEQ ID NO: 32, remains unchanged. In some embodiments, the effector protein comprises an amino acid substitution relative to SEQ ID NO: 32 selected from the group consisting of L26R, E109R, H208R, K184R, K38R, L182R, Q183R, S108R, S198R, and T114R. In some embodiments, the effector protein is a dCas protein. In some embodiments, the dCas protein comprises an amino acid substation D369A, D369N, D658A, D658N, E567A, and E567Q relative to SEQ ID NO: 32. In some embodiments, the guide nucleic acid comprises a nucleotide sequence that is at least 70%, at least 75%, at least 80%, at least 85%, at least 90%, at least 92%, at least 95%, or 100% identical to any one of the sequences set forth in TABLES 8-10. In some embodiments, the guide nucleic acid comprises a spacer sequence that is at least 70%, at least 75%, at least 80%, at least 85%, at least 90%, at least 92%, at least 95%, or 100% identical to any one of the sequences set forth in TABLES 1, 3, and 5 and a repeat sequence that is at least 70%, at least 75%, at least 80%, at least 85%, at least 90%, at least 92%, at least 95%, or 100% identical to the sequence set forth in SEQ ID NOs: 16 or 38-43.

Methods of editing may comprise contacting a target nucleic acid with an effector protein described herein and a guide nucleic acid, wherein the effector protein comprises an amino acid sequence that is at least 70%, at least 75%, at least 80%, at least 85%, at least 90%, at least 95%, at least 97%, or at least 98%, at least 99%, or 100% identical to any one of the sequences set forth in TABLES 15-17. In some embodiments, the effector protein comprises an amino acid sequence that is at least 70%, at least 75%, at least 80%, at least 85%, at least 90%, at least 95%, at least 97%, at least 98%, at least 99%, or 100% identical to any one of the sequences set forth in TABLES 15-17, wherein the amino acid residue at position 220, relative to SEQ ID NO: 773, remains unchanged. In some embodiments, the effector protein comprises an amino acid substitution relative to SEQ ID NO: 773 selected from the group consisting of D220R, N286K, E225K, 180K, S209F, Y315M, N193K, M298L, M295W, A306K, A218K, and K58W. In some embodiments, the effector protein is a dCas protein. In some embodiments, the dCas protein comprises an amino acid substation of E335Q, D237A D418A, D418N, and E335A relative to SEQ ID NO: 773. In some embodiments, the guide nucleic acid comprises a nucleotide sequence that is at least 70%, at least 75%, at least 80%, at least 85%, at least 90%, at least 92%, at least 95%, or 100% identical to any one of the sequences set forth in TABLES 11-13. In some embodiments, the guide nucleic acid comprises a spacer sequence that is at least 70%, at least 75%, at least 80%, at least 85%, at least 90%, at least 92%, at least 95%, or 100% identical to any one of the sequences set forth in TABLES 2, 4, AND 6 and a repeat sequence that is at least 70%, at least 75%, at least 80%, at least 85%, at least 90%, at least 92%, at least 95%, or 100% identical to SEQ ID NO: 488. In some embodiments, the guide nucleic acid comprises a handle sequence that is at least 70%, at least 75%, at least 80%, at least 85%, at least 90%, at least 92%, at least 95%, or 100% identical to SEQ ID NO: 490. In some embodiments, the guide nucleic acid comprises an intermediary sequence that is at least 70%, at least 75%, at least 80%, at least 85%, at least 90%, at least 92%, at least 95%, or 100% identical to SEQ ID NO: 489.

Editing may introduce a mutation (e.g., point mutations, deletions) in a target nucleic acid relative to a corresponding wildtype nucleobase sequence. Editing may remove or correct a disease-causing mutation in a nucleic acid sequence to produce a corresponding wildtype nucleobase sequence. Editing may remove/correct point mutations, deletions, null mutations, or tissue-specific mutations in a target nucleic acid. Editing may be used to generate gene knock-out, gene knock-in, gene editing, gene tagging, or a combination thereof. Methods of the disclosure may be targeted to any locus in a genome of a cell.

Editing may comprise single stranded cleavage, double stranded cleavage, donor nucleic acid insertion, epigenetic modification (e.g., methylation, demethylation, acetylation, or deacetylation), or a combination thereof. In some embodiments, cleavage (single-stranded or double-stranded) is site-specific, meaning cleavage occurs at a specific site in the target nucleic acid, often within the region of the target nucleic acid that hybridizes with the guide nucleic acid spacer region. In some embodiments, the target nucleic acid, and the resulting cleaved nucleic acid is contacted with a nucleic acid for homologous recombination (e.g., homology directed repair (HDR)) or non-homologous end joining (NHEJ). In some cases, a double-stranded break in the target nucleic acid may be repaired (e.g., by NHEJ or HDR) without insertion of a donor template, such that the repair results in an indel in the target nucleic acid at or near the site of the double-stranded break.

In some embodiments, an indel, sometimes referred to as an insertion-deletion or indel mutation, is a type of genetic mutation that results from the insertion and/or deletion of nucleotides in a target nucleic acid. An indel can vary in length (e.g., 1 to 1,000 nucleotides in length) and be detected using methods well known in the art, including sequencing. If the number of nucleotides in the insertion/deletion is not divisible by three, and it occurs in a protein coding region, it is also a frameshift mutation.

In some embodiments, wherein the compositions, systems, and methods of the present disclosure comprise an additional guide nucleic acid or a use thereof, the dual-guided compositions, systems, and methods described herein can modify the target nucleic acid in two locations. In some cases, dual-guided editing can comprise cleavage of the target nucleic acid in the two locations targeted by the guide RNAs. In certain embodiments, upon removal of the sequence between the guide nucleic acids, the wild-type reading frame is restored. A wild-type reading frame can be a reading frame that produces at least a partially, or fully, functional protein. A non-wild-type reading frame can be a reading frame that produces a non-functional or partially non-functional protein.

Accordingly, in some embodiments, compositions, systems, and methods described herein can edit 1 to 1,000 nucleotides or any integer in between, in a target nucleic acid. In certain embodiments, 1 to 1,000, 2 to 900, 3 to 800, 4 to 700, 5 to 600, 6 to 500, 7 to 400, 8 to 300, 9 to 200, or 10 to 100 nucleotides, or any integer in between, can be edited by the compositions, systems, and methods described herein. In some embodiments, 1, 2, 3, 4, 5, 6, 7, 8, 9, 10, or more nucleotides can be edited by the compositions, systems, and methods described herein. In some embodiments, 10, 20, 30, 40, 50, 60, 70, 80 90, 100 or more nucleotides, or any integer in between, can be edited by the compositions, systems, and methods described herein. In some embodiments, 100, 200, 300, 400, 500, 600, 700, 800, 900 or more nucleotides, or any integer in between, can be edited by the compositions, systems, and methods described herein.

In some cases, methods comprise editing a target nucleic acid with two or more effector proteins. Editing a target nucleic acid may comprise introducing a two or more single-stranded breaks in a target nucleic acid. In some embodiments, a break may be introduced by contacting a target nucleic acid with an effector protein and a guide nucleic acid. The guide nucleic acid may bind to the effector protein and hybridize to a region of the target nucleic acid, thereby recruiting the effector protein to the region of the target nucleic acid. Binding of the effector protein to the guide nucleic acid and the region of the target nucleic acid may activate the effector protein, and the effector protein may introduce a break (e.g., a single stranded break) in the region of the target nucleic acid. In some embodiments, modifying a target nucleic acid may comprise introducing a first break in a first region of the target nucleic acid and a second break in a second region of the target nucleic acid. For example, modifying a target nucleic acid may comprise contacting a target nucleic acid with a first guide nucleic acid that binds to a first effector protein and hybridizes to a first region of the target nucleic acid and a second guide nucleic acid that binds to a second programmable nickase and hybridizes to a second region of the target nucleic acid. The first effector protein may introduce a first break in a first strand at the first region of the target nucleic acid, and the second effector protein may introduce a second break in a second strand at the second region of the target nucleic acid. In some embodiments, a segment of the target nucleic acid between the first break and the second break may be removed, thereby modifying the target nucleic acid. In some embodiments, a segment of the target nucleic acid between the first break and the second break may be replaced (e.g., with donor nucleic acid), thereby modifying the target nucleic acid.

Methods, systems, and compositions described herein can edit or modify a target nucleic acid wherein such editing or modification can be measured by indel activity. Indel activity measures the amount of change in a target nucleic acid (e.g., nucleotide deletion(s) and/or insertion(s)) compared to a target nucleic acid that has not been contacted by a polypeptide described in compositions, systems, and methods described herein. For example, indel activity can be detected by next generation sequencing of one or more target loci of a target nucleic acid where indel percentage is calculated as the fraction of sequencing reads containing insertions or deletions relative to an unedited reference sequence. In certain embodiments, methods, systems, and compositions comprising an effector protein and guide nucleic acid described herein can exhibit about 0.0001% to about 65% or more indel activity upon contact to a target nucleic acid compared to a target nucleic acid non-contacted with compositions, systems, or by methods described herein. For example, methods, systems, and compositions comprising an effector protein and guide nucleic acid described herein can exhibit about 0.0001%, about 0.001%, about 0.01%, about 0.1%, about 1%, about 5%, about 10%, about 15%, about 20%, about 25%, about 30%, about 35%, about 40%, about 45%, about 50%, about 55%, about 60%, about 65% or more indel activity.

In certain embodiments, sequence deletion is a modification where one or more sequences in a target nucleic acid are deleted relative to a target nucleic acid without the sequence deletion. In certain embodiments, a sequence deletion can result in or effect a splicing disruption or a frameshift mutation. In certain embodiments, a sequence deletion result in or effect a splicing disruption.

In certain embodiments, a modification is a deletion of an entire exon. In some embodiments, the exon is associated with a disease. In some embodiments, compositions, systems, and methods described herein comprise a combination of a first gRNA, a second gRNA, a first effector protein, and a second effector protein, wherein the combination can be used for deleting the entire exon or a portion thereof. In some embodiments, the first effector protein and the second effector protein are the same. In some embodiments, the first effector protein and the second effector protein are not the same.

In certain embodiments, sequence skipping is a modification where one or more sequences in a target nucleic acid are skipped upon transcription or translation of the target nucleic acid relative to a target nucleic acid without the sequence skipping. In certain embodiments, sequence skipping can result in or effect a splicing disruption or a frameshift mutation. In certain embodiments, sequence skipping can result in or effect a splicing disruption.

In certain embodiments, sequence reframing is a modification where one or more bases in a target are modified so that the reading frame of the sequence is reframed relative to a target nucleic acid without the sequence reframing. In certain embodiments, sequence reframing can result in or effect a splicing disruption or a frameshift mutation. In certain embodiments, sequence reframing can result in or effect a frameshift mutation.

In certain embodiments, sequence knock-in is a modification where one or more sequences is inserted into a target nucleic acid relative to a target nucleic acid without the sequence knock-in. In certain embodiments, sequence knock-in can result in or effect a splicing disruption or a frameshift mutation. In certain embodiments, sequence knock-in can result in or effect a splicing disruption.

In certain embodiments, editing or modification of a target nucleic acid can be locus specific, wherein compositions, systems, and methods described herein can edit or modify a target nucleic acid at one or more specific loci to effect one or more specific mutations comprising splicing disruption mutations, frameshift mutations, sequence deletion, sequence skipping, sequence reframing, sequence knock-in, or any combination thereof. For example, editing or modification of a specific locus can affect any one of a splicing disruption, frameshift (e.g., 1+ or 2+ frameshift), sequence deletion, sequence skipping, sequence reframing, sequence knock-in, or any combination thereof. In certain embodiments, editing or modification of a target nucleic acid can be locus specific, modification specific, or both. In certain embodiments, editing or modification of a target nucleic acid can be locus specific, modification specific, or both, wherein compositions, systems, and methods described herein comprise an effector protein described herein and a guide nucleic acid described herein.

Methods of editing a target nucleic acid or modulating the expression of a target nucleic acid may be performed in vivo. Methods of editing a target nucleic acid or modulating the expression of a target nucleic acid may be performed in vitro. Methods of editing a target nucleic acid or modulating the expression of a target nucleic acid may be performed ex vivo. Editing methods include, but are not limited to, introduction of double stranded breaks (DSB), which can result in deleting some nucleotides and disrupting the translation of a functional protein, base editing, and splice acceptor disruption (SA).

In some embodiments, the method of editing by the effector proteins can be promotor silencing, frameshift mutation, base editing, or splice disruption.

In some embodiments, the editing by the effector protein targets an exon of the APOC3 gene. In some embodiments, the editing by the effector protein targets an intron of the APOC3 gene. In some embodiments, the editing by the effector protein targets the 3′ UTR of the APOC3 gene. In some embodiments, the editing by the effector protein targets the poly-A tail of the APOC3 gene. In some embodiments, the editing by the effector protein decreases transcription of the DNA sequence of the APOC3 gene. In some embodiments, the editing by the effector protein decreases translation of the RNA sequence of the APOC3 gene. In some embodiments, the effector protein targets exon #4 of the APOC3 gene. In some embodiments, the effector protein targets a splice donor site in exon #1 of the APOC3 gene. In some embodiments, the effector protein targets a splice acceptor site in exon #2 of the APOC3 gene. In some embodiments, the effector protein targets a splice donor site in exon #2 of the APOC3 gene. In some embodiments, the effector protein targets a splice acceptor site in exon #3 of the APOC3 gene. In some embodiments, the effector protein targets a splice donor site in exon #3 of the APOC3 gene. In some embodiments, the effector protein targets a splice acceptor site in exon #4 of the APOC3 gene.

A “splice donor site” refers to a gene location that is either 20 base pairs upstream or downstream of the 3′ of an exon junction site. A “splice acceptor site” refers to a gene location that is either 20 base pairs upstream or downstream of the 5′ of an exon junction site.

In some embodiments, the editing by the effector protein targets an exon of the PCSK9 gene. In some embodiments, the editing by the effector protein targets an intron of the PCSK9 gene. In some embodiments, the editing by the effector protein targets the 3′ UTR of the PCSK9 gene. In some embodiments, the editing by the effector protein targets the poly-A tail of the PCSK9 gene. In some embodiments, the editing by the effector protein decreases transcription of the DNA sequence of the PCSK9 gene. In some embodiments, the editing by the effector protein decreases translation of the RNA sequence of the PCSK9 gene. In some embodiments, the effector protein targets exon #1 of the PCSK9 gene. In some embodiments, the effector protein targets exon #2 of the PCSK9 gene. In some embodiments, the effector protein targets exon #3 of the PCSK9 gene. In some embodiments, the effector protein targets exon #4 of the PCSK9 gene. In some embodiments, the effector protein targets exon #5 of the PCSK9 gene. In some embodiments, the effector protein targets exon #6 of the PCSK9 gene. In some embodiments, the effector protein targets exon #7 of the PCSK9 gene. In some embodiments, the effector protein targets exon #8 of the PCSK9 gene. In some embodiments, the effector protein targets exon #9 of the PCSK9 gene. In some embodiments, the effector protein targets exon #10 of the PCSK9 gene. In some embodiments, the effector protein targets exon 1 of the PCSK9 gene. In some embodiments, the effector protein targets exon #11 of the PCSK9 gene. In some embodiments, the effector protein targets exon #12 of the PCSK9 gene.

In some embodiments, the gene regulation is regulated by effector protein repressing a promoter. In some embodiments, the repression is temporary or transient. In some embodiments, the repression is permanent. In some embodiments, the effector protein is linked to a KRAB sequence. In some embodiments, the effector protein is linked to an acetylase sequence. In some embodiments, the effector protein is linked to a methyltransferase. In some embodiments, the effector protein is linked to a Ezh2 sequence.

In some embodiments, the effector protein causes a frameshift mutation. In some embodiments, the effector protein causes the addition of one or more nucleotides causing a shift in the reading frame. In some embodiments, the effector protein causes a deletion of one or more nucleotides causing a shift in the reading frame. In some embodiments, the effector protein causes the deletion or addition of 1, 2, or 4 nucleotides. In some embodiments, the effector protein causes an alternation in the amino acid sequence at protein translation. In some embodiments, the alteration is a missense mutation. In some embodiments, the alteration is a premature stop codon. In some embodiments, the effector protein causes a change in the ribosome reading frame and cause premature termination of translation at a new nonsense or chain termination codon (TAA, TAG, and TGA).

In some embodiments, the effector protein causes a nucleobase to be edited. In some embodiments, the effector protein is linked to an adenine base editing enzyme (e.g., an ABE). In some embodiments, the effector protein is linked to a cytosine base editing enzyme (e.g., a CBE). In some embodiments, the fusion protein causes a cytodine to thymidine transition. In some embodiments, the fusion protein causes a cytodine to uracil transition. In some embodiments, the fusion protein causes a thymidine to cytodine transition. In some embodiments, the fusion protein causes an adenosine to guanosine transition. In some embodiments, the fusion protein causes a guanosine to adenosine conversion. In some embodiments, the alteration results in a missense mutation. In some embodiments, the alteration is a premature stop codon. In some embodiments, the fusion protein causes a premature termination of translation at a new nonsense or chain termination codon (TAA, TAG, and TGA).

### 11. Methods of Treating a Disorder

Described herein are methods for treating and/or preventing a disease in a subject in need thereof comprising administering the systems and compositions described herein. In some embodiments, treating and/or preventing a disease comprises modifying a target nucleic acid in a gene (e.g., APOC3, PCSK9, or ANGPTL3 gene) and/or modifying expression of the gene related to the disease. In some embodiments, the gene related to the disease is APOC3 and the disease is associated with an increase in APOC3 protein expression. In some embodiments, the gene related to the disease is PCSK9 and the disease is associated with an increase in PCSK9 protein expression. In some embodiments, the gene related to the disease is ANGPTL3 and the disease is associated with an increase in ANGPLT3 protein expression.

Described herein are methods for treating or preventing a disease in a subject by modifying a target nucleic acid in a gene (e.g., APOC3, PCSK9, or ANGPTL3) or expression of a gene related to the disease. In some embodiments, the present disclosure provides methods of treating or preventing a disease or disorder in a subject in need thereof comprising administration of the systems and/or compositions described herein. In some embodiments, the disease or disorder comprises an increase in APOC3 expression. In some embodiments, the disease or disorder comprises an increase in PCSK9 expression. In some embodiments, the disease or disorder comprises an increase in ANGPTL3 expression.

In some embodiments, the disease or disorder is a cardiovascular disease. In some embodiments, the present disclosure provides methods of treating or preventing a cardiovascular disease in a subject in need thereof comprising administration of the systems and/or compositions described herein. “Cardiovascular diseases” is an umbrella term that encompasses a broad spectrum of cardiologic diagnoses, affecting heart and circulatory system. Disorders under this term primarily comprise coronary heart diseases, cerebrovascular accidents, and peripheral vascular diseases. The major underlying cause of CVD appears to be atherosclerosis, defined as an immunoinflammatory fibroproliferative disease, in which fatty deposits called atheromatous plaque develops, over many decades, inside the inner layers of the arterial wall, and over time, it narrows the artery depriving the vascularized tissue of oxygen. In some embodiments, the cardiovascular disease is atherosclerotic cardiovascular disease. In some embodiments, the cardiovascular disease is coronary artery disease (CAD). In some embodiments, the disease is chronic kidney disease (CKD). In some embodiments, the disease is Familial chylomicronemia syndrome (FCS). In some embodiments, the disease is lipodystrophy. In some embodiments, the disease is hypertriglyceridemia. In some embodiments, the hypertriglyceridemia is severe hypertriglyceridemia.

In some embodiments, the methods provided herein comprise lowering triglyceride levels in a mammal with hypertriglyceridemia comprising administration of a composition or system described herein. Hypertriglyceridemia (HTG) is a clinical diagnosis defined when plasma triglyceride (TG) concentrations rise above a threshold value, such as the 90th or 95th percentile for age and sex. In some embodiments, the method comprises delivering a composition to the mammal, wherein the composition comprises: a guide nucleic acid comprising a nucleotide sequence that is at least 70%, at least 75%, at least 80%, at least 85%, at least 90%, at least 95%, or 100% identical to a nucleotide sequence selected from any one of SEQ ID NOs: 1-31, 38-43, 67-202, 207-772, 779-820, and 820-2089 and an effector protein or nucleic acid encoding the same, wherein the effector protein comprises an amino acid sequence that is at least 70%, at least 75%, at least 80%, at least 85%, at least 90%, at least 95%, or 100% identical to a nucleotide sequence selected from any one of SEQ ID NOs: 32 and 773.

In some embodiments, the method for treating a disease comprises modifying the APOC3 gene or modifying expression of the APOC3 gene such that the disease (e.g., a cardiovascular disease) is treated. In some embodiments, the gene encodes an APOC3 protein. In some embodiments, the disease is any one of the diseases or disorders listed above and the gene is the gene set forth in TABLE 24.

In some embodiments, the method for treating a disease comprises modifying the PCSK9 gene or modifying expression of the PCSK9 gene such that the disease (e.g., a cardiovascular disease) is treated. In some embodiments, the gene encodes a PCSK9 protein. In some embodiments, the disease is any one of the diseases or disorders listed above and the gene is the gene set forth in TABLE 26.

In some embodiments, the method for treating a disease comprises modifying the ANGPTL3 gene or modifying expression of the ANGPTL3 gene such that the disease (e.g., a cardiovascular disease) is treated. In some embodiments, the gene encodes a ANGPTL3 protein. In some embodiments, the disease is any one of the diseases or disorders listed above and the gene is the gene set forth in TABLE 28.

In some embodiments, methods comprise administering a guide RNA comprising one or more sequences selected from the sequences in TABLES 1-13, or a nucleic acid encoding the same. In some embodiments, methods comprise administering a Cas protein or a nucleic acid encoding the same. In some embodiments, the Cas protein comprises an amino acid sequence that is at least 90% or 95% identical to any one of the sequences in TABLES 15-19. The Cas protein or nucleic acid encoding the same, and the guide RNA or nucleic acid encoding the same may be administered in a single composition. The Cas protein or nucleic acid encoding the same, and the guide RNA or nucleic acid encoding the same may be administered separately (formulaically or chronologically). In some embodiments, methods comprise administering: a Cas protein or a messenger RNA encoding a Cas protein and a lipid nanoparticle; and a viral vector encoding a guide RNA. In some embodiments, methods comprise administering a viral vector encoding the Cas protein and the guide RNA. In some embodiments, methods comprise administering a Cas protein and a lipid nanoparticle. In some embodiments, methods comprise administering a messenger RNA encoding a Cas protein.

In some embodiments, methods comprise administering premedication prior to administering the guide RNA. In some embodiments, the premedication includes a corticosteroid. In some embodiments, the premedication includes a histamine antagonist or inverse agonist. In some embodiments, the premedication includes dexamethasone. In some embodiments, the premedication includes famotidine. In some embodiments, the premedication includes diphenhydramine.

### Further Numbered Embodiments

The present invention is also described, for example and without limitation, in the following numbered embodiments which are not to be construed as limiting the scope thereof in any manner.

Embodiment 1: A guide ribonucleic acid (RNA) or a polynucleotide encoding the same, wherein the guide RNA comprises:
a. a first region comprising a protein binding sequence, and
b. a second region comprising a targeting sequence that is complementary to a target sequence that is within an APOC3 gene, wherein the protein binding sequence is capable of being bound by a clustered regularly interspaced short palindromic repeats (CRISPR) Cas protein other than a Cas9 protein.

Embodiment 2: The guide RNA of embodiment 1, wherein the protein binding sequence comprises a repeat sequence.

Embodiment 3: The guide RNA of any one of embodiments 1-2, wherein the targeting sequence comprises a spacer sequence.

Embodiment 4: The guide RNA of any one of embodiments 1-3, wherein the target sequence comprises at least a portion of an APOC3 exon 1, an APOC3 exon 2, an APOC3 exon 3, an APOC3 exon 4, an APOC3 exon 1 splice donor site, an APOC3 exon 2 splice acceptor site, an APOC3 exon 2 splice donor site, an APOC3 exon 3 splice acceptor site, an APOC3 exon 3 splice acceptor site, an APOC3 exon 4 splice acceptor site, or a combination thereof.

Embodiment 5: The guide RNA of embodiments 1-4, wherein the target sequence is within the exon 4 region of the APOC3 gene.

Embodiment 6: The guide RNA of embodiments 5, wherein the targeting sequence is at least 80%, at least 85%, at least 90%, at least 95% of 100% identical to a sequence selected from SEQ ID NOs: 1-15.

Embodiment 7: The guide RNA of embodiments 1-4, wherein the target sequence comprises a splice donor site of exon 1 of the APOC3 gene.

Embodiment 8: The guide RNA of embodiment 7, wherein the targeting sequence is at least 80%, at least 85%, at least 90%, at least 95% of 100% identical to a sequence selected from SEQ ID NOs: 67-68.

Embodiment 9: The guide RNA of embodiments 1-4, wherein the target sequence comprises a splice acceptor site of exon 2 of the APOC3 gene.

Embodiment 10: The guide RNA of embodiment 9, wherein the targeting sequence is at least 80%, at least 85%, at least 90%, at least 95% of 100% identical to SEQ ID NO: 69.

Embodiment 11: The guide RNA of embodiments 1-4, wherein the target sequence comprises a splice acceptor site of exon 3 of the APOC3 gene.

Embodiment 12: The guide RNA of embodiment 11, wherein the targeting sequence is at least 80%, at least 85%, at least 90%, at least 95% of 100% identical to a sequence selected from SEQ ID NOs: 70-71.

Embodiment 13: The guide RNA of embodiments 1-4, wherein the target sequence comprises a splice acceptor site of exon 4 of the APOC3 gene.

Embodiment 14: The guide RNA of embodiment 13, wherein the targeting sequence is at least 80%, at least 85%, at least 90%, at least 95% of 100% identical to SEQ ID NO: 72.

Embodiment 15: The guide RNA of embodiments 1-4, wherein the target sequence comprises a splice donor site of exon 2 of the APOC3 gene.

Embodiment 16: The guide RNA of embodiment 13, wherein the targeting sequence is at least 80%, at least 85%, at least 90%, at least 95% of 100% identical to SEQ ID NO: 207.

Embodiment 17: The guide RNA of any one of embodiments 1-16, wherein the protein binding sequence is at least 80%, at least 85%, at least 90%, at least 95% or 100% identical to a sequence selected from any one of SEQ ID NOs: 16 or 38-43.

Embodiment 18: The guide RNA of embodiment 17, wherein the guide RNA is selected from the group consisting of SEQ ID NOs: 21, 23, 26, 27, and 31.

Embodiment 19: The guide RNA of any one of embodiments 1-18, wherein the Cas protein is at least 80%, at least 85%, at least 90%, at least 95%, or 100% identical to a sequence selected from Tables 15, 18, and 19.

Embodiment 20: The guide RNA of embodiments 1-4, wherein the target sequence is within the exon 1 region of the APOC3 gene.

Embodiment 21: The guide RNA of embodiment 20, wherein the targeting sequence is at least 80%, at least 85%, at least 90%, at least 95% of 100% identical to a sequence selected from SEQ ID NOs: 209-211.

Embodiment 22: The guide RNA of embodiments 1-4, wherein the target sequence within the exon 2 region of the APOC3 gene.

Embodiment 23: The guide RNA of embodiment 22, wherein the targeting sequence is at least 80%, at least 85%, at least 90%, at least 95% of 100% identical to SEQ ID NO: 212.

Embodiment 24: The guide RNA of embodiments 1-4, wherein the target sequence within the exon 3 region of the APOC3 gene.

Embodiment 25: The guide RNA of embodiment 24, wherein the targeting sequence is at least 80%, at least 85%, at least 90%, at least 95% of 100% identical to a sequence selected from SEQ ID NOs: 213-217.

Embodiment 26: The guide RNA of embodiments 1-4, wherein the target sequence within the exon 4 region of the APOC3 gene.

Embodiment 27: The guide RNA of embodiment 26, wherein the targeting sequence is at least 80%, at least 85%, at least 90%, at least 95% of 100% identical to a sequence selected from SEQ ID NOs: 270-280.

Embodiment 28: The guide RNA of embodiments 1-4, wherein the target sequence comprises a splice acceptor site of exon 3 of the APOC3 gene.

Embodiment 29: The guide RNA of embodiment 28, wherein the targeting sequence is at least 80%, at least 85%, at least 90%, at least 95% of 100% identical to a sequence selected from SEQ ID NOs: 281-290.

Embodiment 30: The guide RNA of embodiments 1-4, wherein the target sequence comprises a splice donor site of exon 3 of the APOC3 gene.

Embodiment 31: The guide RNA of embodiment 30, wherein the targeting sequence is at least 80%, at least 85%, at least 90%, at least 95% of 100% identical to a sequence selected from SEQ ID NOs: 292-296.

Embodiment 32: The guide RNA of embodiments 1-4, wherein the target sequence comprises a splice acceptor site of exon 3 of the APOC3 gene.

Embodiment 33: The guide RNA of embodiment 32, wherein the targeting sequence is at least 80%, at least 85%, at least 90%, at least 95% of 100% identical to SEQ ID NO: 297.

Embodiment 34: The guide RNA of any one of embodiments 1-4, wherein the targeting sequence is at least 80%, at least 85%, at least 90%, at least 95% of 100% identical to SEQ ID NO: 207, 298-299, 804-805, 823-825, 830-1399, 2018-2026, or 2084-2086.

Embodiment 35: The guide RNA of any one of embodiments 1-4 and 20-34, wherein the protein binding sequence is at least 80%, at least 85%, at least 90%, at least 95% or 100% identical to a sequence selected from Table 7.

Embodiment 36: The guide RNA of any one of embodiments 1-4 and 20-35, wherein the Cas protein is at least 80%, at least 85%, at least 90%, at least 95%, or 100% identical to a sequence selected from Tables 15-19.

Embodiment 37: A system comprising the guide RNA of any one of embodiments 1-36, or the polynucleotide encoding the same.

Embodiment 38: The system of embodiment 37, further comprising a Cas protein or a polynucleotide encoding the same.

Embodiment 39: The system of embodiment 38 wherein the polynucleotide is an mRNA polynucleotide.

Embodiment 40: The system of any of embodiments 37-39, wherein the polynucleotide is a DNA expression vector.

Embodiment 41: The system of embodiment 40, wherein the DNA expression vector is an adeno-associated viral (AAV) vector.

Embodiment 42: The system of embodiment 41, comprising a recombinant adeno-associated virus (AAV) expression cassette comprising sequences encoding
a. a first inverted terminal repeat (ITR) and a first promoter;
b. the Cas protein;
c. optionally a second promoter;
d. a second polynucleotide encoding the guide RNA of any one of embodiments 1-36; and
e. a second ITR,
wherein the AAV expression cassette is a self-complementary AAV vector.

Embodiment 43: The system of any one of embodiments 37-42, comprising a lipid or lipid nanoparticle.

Embodiment 44: The system of any one of embodiments 37-43, wherein the Cas protein recognizes a protospacer motif (PAM) of 5′-TTN-3′.

Embodiment 45: The system of embodiment 44, wherein the Cas protein recognizes the PAM sequence selected from the group consisting of 5′-TTG-3′, 5′-TTC-3′, 5′-TTT-3′, and 5′-TTA-3′.

Embodiment 46: The system of any one of embodiments 37-45, wherein the Cas protein comprises a sequence that is at least 80%, at least 85%, at least 90%, at least 95%, at least 97%, at least 98%, at least 99%, or 100% identical to SEQ ID NO: 32.

Embodiment 47: The system of embodiment 46, wherein the Cas protein has a positively charged amino acid at position 26 of SEQ ID NO: 32 and/or a threonine at position 471 of SEQ ID NO: 32.

Embodiment 48: The system of embodiment 47, wherein the positively charged amino acid is selected from arginine, histidine, and lysine.

Embodiment 49: The system of embodiment 48, wherein the positively charged amino acid is arginine.

Embodiment 50: The system of any one of embodiments 37-43, wherein the Cas protein recognizes a protospacer motif (PAM) of 5′-TNTR-3′.

Embodiment 51: The system of embodiment 50, wherein the Cas protein recognizes the PAM sequence selected from the group consisting of 5′-TTTG-3′, 5′-TCTG-3′, 5′-TGTG-3′, 5′-TCTA-3′, 5′-TATA-3′, 5′-TTTA-3′, 5′-TGTA-3′, and 5′-TATG-3′.

Embodiment 52:The system of any one of embodiments 37-43 and 50-51, wherein the Cas protein comprises a sequence that is at least 80%, at least 85%, at least 90%, at least 95%, at least 97%, at least 98%, at least 99%, or 100% identical to SEQ ID NO: 773.

Embodiment 53: The system of embodiment 52, wherein the Cas protein has a positively charged amino acid at position 220 of SEQ ID NO: 773.

Embodiment 54: The system of embodiment 53, wherein the positively charged amino acid is selected from arginine, histidine, and lysine.

Embodiment 55: The system of embodiment 54, wherein the positively charged amino acid is arginine.

Embodiment 56: The system of any one of embodiments 37-55, wherein the Cas protein amino acid sequence comprises a nuclear localization signal.

Embodiment 57: The system of any one of embodiments 37-56, wherein the Cas protein amino acid sequence is at least 80%, at least 85%, at least 90%, at least 95%, at least 97%, at least 98%, at least 99%, or 100% identical to a sequence from Tables 15-19.

Embodiment 58: The system of any one of embodiments 37-57, wherein the system further comprises an additional guide RNA that binds a different portion of the target nucleic acid than the guide RNA.

Embodiment 59: The system of any one of embodiments 37-58, wherein the Cas protein reduces expression of the APOC3 gene.

Embodiment 60: The system of any one of embodiments 37-59, wherein the Cas protein is linked to a heterologous protein.

Embodiment 61: The system of embodiment 60, wherein the heterologous protein is linked to the N-terminus or C-terminus of the Cas protein.

Embodiment 62: The system of any of embodiments 58-61, wherein the Cas protein is linked to a KRAB domain, acetylase domain, or a base editing enzyme.

Embodiment 63: The system of embodiment 62, wherein the base editing enzyme is a cytosine base editing enzyme (CBE), adenine base editing enzyme (ABE), or a C-to-G base editing enzyme (CGBE).

Embodiment 64: The system of embodiment 59, wherein the expression of the APOC3 gene is reduced by promoter inhibition, a frameshift mutation, base editing, and/or 3′ UTR disruption.

Embodiment 65: The system of any of embodiments 59 or 64, wherein the reduced expression of the APOC3 gene is transient or permanent.

Embodiment 66: A pharmaceutical composition comprising the guide RNA of any one of embodiments 1-36 or the system of any one of embodiments 37-65, and a pharmaceutical acceptable carrier.

Embodiment 67: A cell, or population of cells, comprising or modified by the guide RNA of any one of embodiments 1-36 or the system of any one of embodiments 37-65.

Embodiment 68: A method of modifying an APOC3 gene, comprising contacting the APOC3 gene with the guide RNA of any one of embodiments 1-36 or system of any one of embodiments 37-65.

Embodiment 69: The method of embodiment 68, wherein modifying of the APOC3 gene comprises inserting, deleting, or substituting one or more nucleotides in the APOC3 gene.

Embodiment 70: The method of embodiment 69, wherein the modifying of the APOC3 gene reduces the expression of the APOC3 gene.

Embodiment 71: The method of embodiment 70, wherein the reduced expression of the APOC3 gene is transient.

Embodiment 72: The method of embodiments 70, wherein the reduced expression of the APOC3 gene is permanent.

Embodiment 73: A nucleic acid expression vector that encodes a guide RNA, wherein the guide RNA comprises at least one sequence that is at least 80%, at least 85%, at least 90%, at least 95% or 100% identical to a sequence selected from any one of TABLES 1, 2, 7, 8, or 11.

Embodiment 74: The nucleic acid expression vector of embodiment 73, wherein the nucleic acid expression vector is an adenoviral associated viral (AAV) vector.

Embodiment 75: The nucleic acid expression vector of embodiments 73 or 74, wherein the nucleic acid expression vector further comprises a polynucleotide encoding an effector protein that is at least 80%, at least 85%, at least 90%, at least 95%, at least 99%, or 100% identical to any one of the sequences recited in TABLES 15-19.

Embodiment 76: A pharmaceutical composition, comprising the nucleic acid expression vector of any one of embodiments 73-75, and a pharmaceutically acceptable excipient.

Embodiment 77: A system comprising the nucleic acid expression vector of any one of embodiments 73-75.

Embodiment 78: The system of embodiment 77, comprising at least one detection reagent for detecting a target nucleic acid.

Embodiment 79: A method of modifying an APOC3 gene, the method comprising contacting the APOC3 gene genome with the nucleic acid expression vector of any one of embodiments 73-75, the pharmaceutical composition of embodiment 76, or the system of any one of embodiments 77-78, thereby modifying the APOC3 gene.

Embodiment 80: The method of embodiment 79, wherein the modifying of the APOC3 gene comprises cleaving the APOC3 gene, deleting a nucleotide of the APOC3 gene, inserting a nucleotide into the APOC3 gene, substituting a nucleotide of the APOC3 gene with an alternative nucleotide, or editing a nucleotide, more than one of the foregoing, or any combination thereof.

Embodiment 81: The method of embodiments 79 or 80, wherein the composition further comprises an additional guide RNA that binds a different portion of the APOC3 gene than the guide RNA.

Embodiment 82: The method of embodiment 81, wherein the composition removes the sequence between the guide RNA and the additional guide RNA.

Embodiment 83: The method of any one of embodiments 79-82, further comprising contacting the APOC3 gene with a donor nucleic acid.

Embodiment 84: The method of any one of embodiments 79-83, wherein the method is performed in a cell.

Embodiment 85: The method of embodiment 84, wherein the method is performed in vivo.

Embodiment 86: A cell comprising the nucleic acid expression vector of any one of embodiments 73-75.

Embodiment 87: A cell that comprises a target nucleic acid modified by the nucleic acid expression vector of any one of embodiments 73-75.

Embodiment 88: The cell of embodiments 86 or 87, wherein the cell is a eukaryotic cell.

Embodiment 89: The cell of any one of embodiments 86-88, wherein the cell is a mammalian cell.

Embodiment 90: The cell of any one of embodiments 86-89, wherein the cell is a human cell.

Embodiment 91: A population of cells that comprises at least one cell of any one of embodiments 86-90.

Embodiment 92: A method of treating a disease caused by a misexpression of the APOC3 gene, the method comprising contacting a cell that has the misexpression of the APOC3 gene, comprising contacting the APOC3 gene with the guide RNA of any of embodiments 1-36 or system of any one of embodiments 37-65.

Embodiment 93: The method of embodiment 92, comprising modifying the APOC3 gene.

Embodiment 94: The method of embodiment 93, wherein modifying the APOC3 gene comprises inserting, deleting, or substituting one or more nucleotides in the APOC3 gene.

Embodiment 95: The method of any one of embodiments 92-94, wherein the disease is a cardiovascular disease.

Embodiment 96: The method of embodiment 95, wherein the cardiovascular disease is atherosclerotic cardiovascular disease or is coronary artery disease (CAD).

Embodiment 97: The method of any one of embodiments 92-94, wherein the disease is chronic kidney disease (CKD).

Embodiment 98: The method of any one of embodiments 92-94, wherein the disease is familial chylomicronemia syndrome (FCS).

Embodiment 99: The method of any one of embodiments 92-94, wherein the disease is lipodystrophy.

Embodiment 100: The method of any one of embodiments 92-94, wherein the disease is hypertriglyceridemia.

Embodiment 101: The method of any one of embodiment 100, wherein the disease is severe hypertriglyceridemia.

Embodiment 102: A system comprising a recombinant adeno-associated virus (AAV) expression cassette comprising sequences encoding
a. a first inverted terminal repeat (ITR) and a first promoter;
b. a Cas protein comprising a sequence that is at least 95% identical to any of SEQ ID NOs: 32-35, 45-46, or 54-66;
c. optionally a second promoter;
d. a second polynucleotide encoding SEQ ID NO:26; and
e. a second ITR,
wherein the AAV expression cassette is a self-complementary AAV vector.

Embodiment 103: A composition for introducing indels in an APOC3 gene in eukaryotic cells or organisms comprising SEQ ID NO: 26 or a nucleic acid encoding the same, and a Cas protein comprising any of SEQ ID NOs: 32-35, 45-46, or 54-66 or nucleic acid encoding the same.

Embodiment 104: A guide ribonucleic acid (RNA) or a polynucleotide encoding the same, wherein the guide RNA comprises:
a. a first region comprising a protein binding sequence, and
b. a second region comprising a targeting sequence that is complementary to a target sequence that is within a PCSK9 gene,
wherein the protein binding sequence is capable of being bound by a clustered regularly interspaced short palindromic repeats (CRISPR) Cas protein other than a Cas9 protein.

Embodiment 105: The guide RNA of embodiment 104, wherein the protein binding sequence comprises a repeat sequence.

Embodiment 106: The guide RNA of any one of embodiments 104-105, wherein the targeting sequence comprises a spacer sequence.

Embodiment 107: The guide RNA of any one of embodiments 105-106, wherein the target sequence comprises at least a portion of a PCSK9 exon 1, PCSK9 exon 2, PCSK9 exon 3, PCSK9 exon 4, PCSK9 exon 5, PCSK9 exon 6, PCSK9 exon 7, PCSK9 exon 8, PCSK9 exon 9, PCSK9 exon 10, PCSK9 exon 11, PCSK9 exon 12, or a combination thereof.

Embodiment 108: The guide RNA of embodiment 107, wherein the targeting sequence is at least 80%, at least 85%, at least 90%, at least 95% of 100% identical to a sequence selected from SEQ ID NOs: 79-140, 208, 300-487, 799-803, 809, 822, and 1970-1995.

Embodiment 109: The guide RNA of any one of embodiments 104-108, wherein the protein binding sequence is at least 80%, at least 85%, at least 90%, at least 95% or 100% identical to a sequence selected from Table 7.

Embodiment 110: The guide RNA of any one of embodiments 104-109, wherein the Cas protein is at least 80%, at least 85%, at least 90%, at least 95%, or 100% identical to a sequence selected from TABLES 15-19.

Embodiment 111: A guide ribonucleic acid (RNA) or a polynucleotide encoding the same, wherein the guide RNA comprises:
a. a first region comprising a protein binding sequence, and
b. a second region comprising a targeting sequence that is complementary to a target sequence that is within a ANGPLT3 gene,
   wherein the protein binding sequence is capable of being bound by a clustered regularly interspaced short palindromic repeats (CRISPR) Cas protein other than a Cas9 protein.

Embodiment 112: The guide RNA of embodiment 111, wherein the protein binding sequence comprises a repeat sequence.

Embodiment 113: The guide RNA of any one of embodiments 111-112, wherein the targeting sequence comprises a spacer sequence.

Embodiment 114: The guide RNA of embodiment 113, wherein the targeting sequence is at least 80%, at least 85%, at least 90%, at least 95% of 100% identical to a sequence selected from SEQ ID NOs: 806-808 or 1996-2017.

Embodiment 115: The guide RNA of any one of embodiments 111-114, wherein the protein binding sequence is at least 80%, at least 85%, at least 90%, at least 95% or 100% identical to a sequence selected from Table 7.

Embodiment 116: The guide RNA of any one of embodiments 111-115, wherein the Cas protein is at least 80%, at least 85%, at least 90%, at least 95%, or 100% identical to a sequence selected from TABLES 15-19.

Embodiment 117: A method of treating a disease caused by a misexpression of the PCSK9 gene or the ANGPTL3 gene, the method comprising contacting a cell that has the misexpression of the PCSK9 gene or the ANGPTL3 gene, comprising contacting the PCSK9 gene or ANGPTL3 gene with the guide RNA of any of embodiments 104-116.

Embodiment 118: The method of embodiment 117, comprising modifying the PCSK9 gene or the ANGPTL3 gene.

Embodiment 119: The method of embodiment 118, wherein modifying the PCSK9 gene or the ANGPTL3 gene comprises inserting, deleting, or substituting one or more nucleotides in the PCSK9 gene or the ANGPTL3 gene.

Embodiment 120: The method of any one of embodiments 117-119, wherein the disease is a cardiovascular disease.

Embodiment 121: The method of embodiment 120, wherein the cardiovascular disease is atherosclerotic cardiovascular disease or is coronary artery disease (CAD).

Embodiment 122: A fusion protein comprising an effector protein and a base editing enzyme, wherein
a. the effector protein comprises an amino acid sequence that is at least 65%, at least 70%, at least 75%, at least 80%, at least 85%, at least 90%, at least 95%, at least 97%, at least 98%, at least 99%, or 100% identical to SEQ ID NO: 32; and
b. the base editing enzyme comprises an amino acid sequence that is at least 65%, at least 70%, at least 75%, at least 80%, at least 85%, at least 90%, at least 95%, at least 97%, at least 98%, at least 99%, or 100% identical to SEQ ID NO: 796.

Embodiment 123: The fusion protein of embodiment 122, wherein the effector protein comprises the amino acid substitutions of L26K and E567Q relative to SEQ ID NO: 32.

Embodiment 124: The fusion protein of any one of embodiments 122 or 123, wherein the fusion protein comprises an amino acid sequence that is at least 90% or at least 95% identical to SEQ ID NO: 798.

Embodiment 125: The fusion protein of embodiments 122 or 123, wherein the fusion protein comprises or consists of SEQ ID NO: 798.

Embodiment 126: A system comprising
a. a guide nucleic acid or a DNA molecule encoding the guide nucleic acid, wherein the guide nucleic acid comprises:
   i. a first region comprising a protein binding sequence; and
   ii. a second region comprising a targeting sequence that is complementary to a target sequence of an APOC3 gene and comprising a spacer sequence selected from SEQ ID NOs: 804-805,
   wherein the first region is located 5′ of the second region;
b. a fusion protein comprising an effector protein and a base editing enzyme, or a nucleic acid encoding the fusion protein.

Embodiment 127: A system comprising
a. a guide nucleic acid or a DNA molecule encoding the guide nucleic acid, wherein the guide nucleic acid comprises a nucleotide sequence that is at least 70%, at least 75%, at least 80%, at least 85%, at least 90%, at least 95%, or 100% identical to a sequence selected from SEQ ID NOs: 815-816; and
b. a fusion protein comprising an effector protein and a base editing enzyme, or a nucleic acid encoding the fusion protein, wherein the fusion protein comprises an amino acid sequence that is at least 90% or at least 95% identical to SEQ ID NO: 798.

Embodiment 128: A system comprising
a. a guide nucleic acid or a DNA molecule encoding the guide nucleic acid, wherein the guide nucleic acid comprises:
   i. a first region comprising a protein binding sequence; and
   ii. a second region comprising a targeting sequence that is complementary to a target sequence of an PCSK9 gene and is selected from SEQ ID NOs: 799-803 and 809,
   wherein the first region is located 5′ of the second region;
b. a fusion protein comprising an effector protein and a base editing enzyme, or a nucleic acid encoding the fusion protein.

Embodiment 129: A system comprising
a. a guide nucleic acid or a DNA molecule encoding the guide nucleic acid, wherein the guide nucleic acid comprises a nucleotide sequence that is at least 70%, at least 75%, at least 80%, at least 85%, at least 90%, at least 95%, or 100% identical to a sequence selected from SEQ ID NOs: 810-814 and 820; and
b. a fusion protein comprising an effector protein and a base editing enzyme, or a nucleic acid encoding the fusion protein, wherein the fusion protein comprises an amino acid sequence acid sequence that is at least 90% or at least 95% identical to SEQ ID NO: 798.

Embodiment 130: A system comprising
a. a guide nucleic acid or a DNA molecule encoding the guide nucleic acid, wherein the guide nucleic acid comprises:
   i. a first region comprising a protein binding sequence; and
   ii. a second region comprising a targeting sequence that is complementary to a target sequence of an ANGPTL gene and comprising a spacer sequence selected from SEQ ID NOs: 806-808,
   wherein the first region is located 5′ of the second region;
b. a fusion protein comprising an effector protein and a base editing enzyme, or a nucleic acid encoding the fusion protein.

Embodiment 131:A system comprising
a. a guide nucleic acid or a DNA molecule encoding the guide nucleic acid, wherein the guide nucleic acid comprises a nucleotide sequence that is at least 70%, at least 75%, at least 80%, at least 85%, at least 90%, at least 95%, or 100% identical to a sequence selected from SEQ ID NOs: 817-819; and
b. a fusion protein comprising an effector protein and a base editing enzyme, or a nucleic acid encoding the fusion protein, wherein the fusion protein comprises an amino acid sequence acid sequence that is at least 90% or at least 95% identical to SEQ ID NO: 798.

Embodiment 132: A fusion protein comprising an effector protein and a base editing enzyme, wherein
a. the effector protein comprises an amino acid sequence that is at least 65%, at least 70%, at least 75%, at least 80%, at least 85%, at least 90%, at least 95%, at least 97%, at least 98%, at least 99%, or 100% identical to SEQ ID NO: 773; and
b. the base editing enzyme comprises an amino acid sequence that is at least 65%, at least 70%, at least 75%, at least 80%, at least 85%, at least 90%, at least 95%, at least 97%, at least 98%, at least 99%, or 100% identical to SEQ ID NO: 796.

Embodiment 133: The fusion protein of embodiment 132, wherein the effector protein comprises the amino acid substitutions of D220R and E335Q relative to SEQ ID NO: 773.

Embodiment 134: The fusion protein of any one of embodiments 132 or 133, wherein the fusion protein comprises an amino acid sequence that is at least 90% or at least 95% identical to SEQ ID NO: 797.

Embodiment 135: The fusion protein of any one of embodiments 132 or 133, wherein the fusion protein comprises or consists of SEQ ID NO: 797.

Embodiment 136: A system comprising
a. a guide nucleic acid or a DNA molecule encoding the guide nucleic acid, wherein the guide nucleic acid comprises:
   i. a first region comprising a protein binding sequence; and
   ii. a second region comprising a targeting sequence that is complementary to a target sequence of an APOC3 gene and comprising a spacer sequence selected from TABLES 1 and 2,
   wherein the first region is located 5′ of the second region;
b. a fusion protein comprising an effector protein and a base editing enzyme, or a nucleic acid encoding the fusion protein.

Embodiment 137: A system comprising
a. a guide nucleic acid or a DNA molecule encoding the guide nucleic acid, wherein the guide nucleic acid comprises:
   i. a first region comprising a protein binding sequence; and
   ii. a second region comprising a targeting sequence that is complementary to a target sequence of an PCSK9 gene and comprising a spacer sequence selected from TABLES 3 and 4,
   wherein the first region is located 5′ of the second region;
b. a fusion protein comprising an effector protein and a base editing enzyme, or a nucleic acid encoding the fusion protein.

Embodiment 138: A system comprising
a. a guide nucleic acid or a DNA molecule encoding the guide nucleic acid, wherein the guide nucleic acid comprises:
   i. a first region comprising a protein binding sequence; and
   ii. a second region comprising a targeting sequence that is complementary to a target sequence of an ANGPTL3 gene and comprising a spacer sequence selected from TABLES 5 and 6,
   wherein the first region is located 5′ of the second region;
b. a fusion protein comprising an effector protein and a base editing enzyme, or a nucleic acid encoding the fusion protein.

Embodiment 139: A system comprising
a. a guide nucleic acid or a DNA molecule encoding the guide nucleic acid, wherein the guide nucleic acid comprises a nucleotide sequence that is at least 70%, at least 75%, at least 80%, at least 85%, at least 90%, at least 95%, or 100% identical to a sequence selected from TABLES 8-10; and
b. a fusion protein comprising an effector protein and a base editing enzyme, or a nucleic acid encoding the fusion protein, wherein the fusion protein comprises an amino acid sequence that is at least 90% or at least 95% identical to SEQ ID NO: 798.

Embodiment 140: A system comprising
a. a guide nucleic acid or a DNA molecule encoding the guide nucleic acid, wherein the guide nucleic acid comprises a nucleotide sequence that is at least 70%, at least 75%, at least 80%, at least 85%, at least 90%, at least 95%, or 100% identical to a sequence selected from TABLES 11-13; and
b. a fusion protein comprising an effector protein and a base editing enzyme, or a nucleic acid encoding the fusion protein, wherein the fusion protein comprises an amino acid sequence that is at least 90% or at least 95% identical to SEQ ID NO: 797.

Embodiment 141: A method of reducing triglycerides in a subject in need thereof, the method comprising administering: (a) an effector protein or a nucleic acid encoding an effector protein, wherein the effector protein comprises an amino acid sequence that is at least 90%, at least 95%, at least 98%, at least 99% or 100% identical to an amino acid sequence selected from SEQ ID NO: 32 and SEQ ID NO: 773; and (b) a guide nucleic acid comprising a spacer sequence that hybridizes to a target sequence in the human APOC3 gene.

Embodiment 142: The method of embodiment 141, wherein the subject has hypertriglyceridemia, hypercholesterolemia, or a combination thereof.

Embodiment 143: The method of any one of embodiments 141 or 142, wherein the effector protein is described in TABLES 15-19.

Embodiment 144: A composition comprising
a) a fusion protein or a nucleic acid encoding the fusion protein, wherein the fusion protein comprises:
   i. an effector protein; and
   ii. a methyltransferase; and
b) a guide RNA or a nucleic acid encoding the guide RNA, wherein the guide RNA comprises:
   i. a first region comprising a protein binding sequence; and
   ii. a second region comprising a spacer sequence that hybridizes to
a target sequence of an APOC3 gene.

Embodiment 145: A composition or system comprising a guide ribonucleic acid (RNA) or a polynucleotide encoding the same, wherein the guide RNA comprises:
a) a first region comprising a protein binding sequence, and
b) a second region comprising a targeting sequence that is complementary to a target sequence that is within an APOC3 gene,
   wherein the target sequence is adjacent to a protospacer adjacent motif (PAM) selected from 5′-NTTN-3′ and 5′-NNTN-3′.

Embodiment 146: The composition or system of embodiment 145, wherein the targeting sequence comprises a nucleotide sequence that is at least 70%, at least 75%, at least 80%, at least 85%, at least 90%, at least 95% or 100% identical to any one of SEQ ID NOs: 1-15, 67-72, 207, 209-299, 804-805, 823-825, 830-1399, 2018-2026, and 2084-2086.

Embodiment 147: The composition or system of any one of embodiments 145-146, wherein the PAM is 5′-NTTN-3′ and wherein
a) the targeting sequence comprises a nucleotide sequence that is at least 70%, at least 75%, at least 80%, at least 85%, at least 90%, at least 95% or 100% identical to any one of SEQ ID NOs: 1-15, 67-72, 207, 804-805, and 830-999, and
b) the protein binding sequence comprises a nucleotide sequence that is at least 70%, at least 75%, at least 80%, at least 85%, at least 90%, at least 95% or 100% identical to any one of SEQ ID NOs: 16 and 38-43.

Embodiment 148: The composition or system of embodiment 147, wherein the composition or system comprises an effector protein or a nucleic acid encoding the same, wherein the effector protein comprises an amino acid sequence that is at least 70%, at least 75%, at least 80%, at least 85%, at least 90%, at least 95%, at least 97%, at least 98%, at least 99%, or 100% identical to any one of SEQ ID NOs: 32, 34, 794, or 2090.

Embodiment 149: The composition or system of any one of embodiments 145-148, wherein the guide RNA comprises a nucleotide sequence that is at least 70%, at least 75%, at least 80%, at least 85%, at least 90%, at least 95% or 100% identical to any one of SEQ ID NOs: 17-31, 73-78, 491, 815-816, and 1400-1569.

Embodiment 150: The composition or system of embodiments 145 or 146, wherein the PAM is 5′-NNTN-3′, and wherein
a) the targeting sequence comprises a nucleotide sequence that is at least 70%, at least 75%, at least 80%, at least 85%, at least 90%, at least 95% or 100% identical to any one of SEQ ID NOs: 209-299, 823-825, 1000-1399, 2018-2026, and 2084-2086, and
b) the protein binding sequence comprises a nucleotide sequence that is at least 70%, at least 75%, at least 80%, at least 85%, at least 90%, at least 95% or 100% identical to SEQ ID NO: 488.

Embodiment 151: The composition or system of embodiment 150, wherein the protein binding sequence further comprises a nucleotide sequence that is at least 70%, at least 75%, at least 80%, at least 85%, at least 90%, at least 95% or 100% identical to SEQ ID NOs: 489 or 490.

Embodiment 152: The composition or system of embodiment 150 or embodiment 151, wherein the composition or system comprises an effector protein or a nucleic acid encoding the same, wherein the effector protein comprises an amino acid sequence that is at least 70%, at least 75%, at least 80%, at least 85%, at least 90%, at least 95%, at least 97%, at least 98%, at least 99%, or 100% identical to any one of SEQ ID NOs: 773, 775, or 793.

Embodiment 153: The composition or system of any one of embodiments 145, 146, and 150-152, wherein the guide RNA comprises a nucleotide sequence that is at least 70%, at least 75%, at least 80%, at least 85%, at least 90%, at least 95% or 100% identical to any one of SEQ ID NOs: 494-584, 826-828, 1570-1969, 2075-2083, and 2087-2089.

Embodiment 154: A composition or system comprising a guide ribonucleic acid (RNA) or a polynucleotide encoding the same, wherein the guide RNA comprises:
a) a first region comprising a protein binding sequence, and
b) a second region comprising a targeting sequence that is complementary to a target sequence that is within a PCSK9 gene, wherein the target sequence is adjacent to a protospacer adjacent motif (PAM) selected from 5′-NTTN-3′ and 5′-NNTN-3′.

Embodiment 155: The composition or system of embodiment 154, wherein the targeting sequence comprises a nucleotide sequence that is at least 70%, at least 75%, at least 80%, at least 85%, at least 90%, at least 95% or 100% identical to any one of SEQ ID NOs: 79-140, 208, 300-487, 799-803, 809, 822, and 1970-1995.

Embodiment 156: The composition or system of any one of embodiments 154-155, wherein the PAM is 5′-NTTN-3′ and wherein
a) the targeting sequence comprises a nucleotide sequence that is at least 70%, at least 75%, at least 80%, at least 85%, at least 90%, at least 95% or 100% identical to any one of SEQ ID NOs: 79-140, 208, 799-803, and 809, and
b) the protein binding sequence comprises a nucleotide sequence that is at least 70%, at least 75%, at least 80%, at least 85%, at least 90%, at least 95% or 100% identical to any one of SEQ ID NOs: 16 and 38-43.

Embodiment 157: The composition or system of embodiment 156, wherein the composition or system comprises an effector protein or a nucleic acid encoding the same, wherein the effector protein comprises an amino acid sequence that is at least 70%, at least 75%, at least 80%, at least 85%, at least 90%, at least 95%, at least 97%, at least 98%, at least 99%, or 100% identical to any one of SEQ ID NOs: 32, 34, 794, or 2090.

Embodiment 158: The composition or system of any one of embodiments 154-157, wherein the guide RNA comprises a nucleotide sequence that is at least 70%, at least 75%, at least 80%, at least 85%, at least 90%, at least 95% or 100% identical to any one of SEQ ID NOs: 141-202, 492-493, 810-814, 820.

Embodiment 159: The composition or system of embodiments 154 or 155, wherein the PAM is 5′-NNTN-3′, and wherein
a) the targeting sequence comprises a nucleotide sequence that is at least 70%, at least 75%, at least 80%, at least 85%, at least 90%, at least 95% or 100% identical to any one of SEQ ID NOs: 300-487, 822, and 1970-1995, and
b) the protein binding sequence comprises a nucleotide sequence that is at least 70%, at least 75%, at least 80%, at least 85%, at least 90%, at least 95% or 100% identical to SEQ ID NO: 488.

Embodiment 160: The composition or system of embodiment 159, wherein the protein binding sequence further comprises a nucleotide sequence that is at least 70%, at least 75%, at least 80%, at least 85%, at least 90%, at least 95% or 100% identical to SEQ ID NOs: 489 or 490.

Embodiment 161: The composition or system of embodiments 159 or 160, wherein the composition or system comprises an effector protein or a nucleic acid encoding the same, wherein the effector protein comprises an amino acid sequence that is at least 70%, at least 75%, at least 80%, at least 85%, at least 90%, at least 95%, at least 97%, at least 98%, at least 99%, or 100% identical to any one of SEQ ID NOs: 773, 775, or 793.

Embodiment 162: The composition or system of any one of embodiments 154, 155, and 159-161, wherein the guide RNA comprises a nucleotide sequence that is at least 70%, at least 75%, at least 80%, at least 85%, at least 90%, at least 95% or 100% identical to any one of SEQ ID NOs: 585-772, 829, and 2027-2052.

Embodiment 163: A composition or system comprising a guide ribonucleic acid (RNA) or a polynucleotide encoding the same, wherein the guide RNA comprises:
a) a first region comprising a protein binding sequence, and
b) a second region comprising a targeting sequence that is complementary to a target sequence that is within a ANGPTL3 gene,
   wherein the target sequence is adjacent to a protospacer adjacent motif (PAM) selected from 5′-NTTN-3′ and 5′-NNTN-3′.

Embodiment 164: The composition or system of embodiment 163, wherein the targeting sequence comprises a nucleotide sequence that is at least 70%, at least 75%, at least 80%, at least 85%, at least 90%, at least 95% or 100% identical to any one of SEQ ID NOs: 806-808 and 1996-2017.

Embodiment 165: The composition or system of any one of embodiments 163-165, wherein the PAM is 5′-NTTN-3′ and wherein
a) the targeting sequence comprises a nucleotide sequence that is at least 70%, at least 75%, at least 80%, at least 85%, at least 90%, at least 95% or 100% identical to any one of SEQ ID NOs: 806-808, and
b) the protein binding sequence comprises a nucleotide sequence that is at least 70%, at least 75%, at least 80%, at least 85%, at least 90%, at least 95% or 100% identical to any one of SEQ ID NOs: 16 and 38-43.

Embodiment 166: The composition or system of embodiments 165, wherein the composition or system comprises an effector protein or a nucleic acid encoding the same, wherein the effector protein comprises an amino acid sequence that is at least 70%, at least 75%, at least 80%, at least 85%, at least 90%, at least 95%, at least 97%, at least 98%, at least 99%, or 100% identical to any one of SEQ ID NOs: 32, 34, 794, or 2090.

Embodiment 167: The composition or system of any one of embodiments 163-166, wherein the guide RNA comprises a nucleotide sequence that is at least 70%, at least 75%, at least 80%, at least 85%, at least 90%, at least 95% or 100% identical to any one of SEQ ID NOs: 817-819.

Embodiment 168: The composition or system of embodiments 166 or 167, wherein the PAM is 5′-NNTN-3′, and wherein
a) the targeting sequence comprises a nucleotide sequence that is at least 70%, at least 75%, at least 80%, at least 85%, at least 90%, at least 95% or 100% identical to any one of SEQ ID NOs: 1996-2017, and
b) the protein binding sequence comprises a nucleotide sequence that is at least 70%, at least 75%, at least 80%, at least 85%, at least 90%, at least 95% or 100% identical to SEQ ID NO: 488.

Embodiment 169: The composition or system of embodiment 168, wherein the protein binding sequence further comprises a nucleotide sequence that is at least 70%, at least 75%, at least 80%, at least 85%, at least 90%, at least 95% or 100% identical to SEQ ID NOs: 489 or 490.

Embodiment 170: The composition or system of embodiments 168 or 169, wherein the composition or system comprises an effector protein or a nucleic acid encoding the same, wherein the effector protein comprises an amino acid sequence that is at least 70%, at least 75%, at least 80%, at least 85%, at least 90%, at least 95%, at least 97%, at least 98%, at least 99%, or 100% identical to SEQ ID NOs: 773, 775, or 793.

Embodiment 171: The composition or system of any one of embodiments 163, 164, and 168-170, wherein the guide RNA comprises a nucleotide sequence that is at least 70%, at least 75%, at least 80%, at least 85%, at least 90%, at least 95% or 100% identical to any one of SEQ ID NOs: 2053-2074.

Embodiment 172: The composition or system of any of embodiments 148, 149, 152, 153, 157, 158, 161, 162, 166, 167, 170, or 171 wherein the effector protein is fused to an effector partner protein, optionally wherein the effector partner protein is selected from a deaminase, a reverse transcriptase, a recombinase, and a methyltransferase.

Embodiment 173: The composition or system of any of embodiments 152, 161, or 170, wherein the targeting sequence is at least 80%, at least 85%, at least 90%, at least 95%, or 100% identical to a sequence selected from SEQ ID NOs: 1970-2026, wherein the effector protein is at least 80%, at least 85%, at least 90%, at least 95%, or 100% identical to any one of SEQ ID NOs: 773, 775, or 793, and wherein the effector protein is fused to a base editing enzyme.

Embodiment 174: The composition or system of embodiment 148, wherein the targeting sequence is at least 80%, at least 85%, at least 90%, at least 95%, or 100% identical to a sequence selected from SEQ ID NOs: 830-999, wherein the effector protein is at least 80%, at least 85%, at least 90%, at least 95%, or 100% identical to any one of SEQ ID NOs: 32, 34, 794, or 2090 and wherein the effector protein is fused to a KRAB domain, a methyltransferase, or a combination thereof.

Embodiment 175: The composition or system of embodiment 152, wherein the targeting sequence is at least 80%, at least 85%, at least 90%, at least 95%, or 100% identical to a sequence selected from SEQ ID NOs: 1000-1399, wherein the effector protein is at least 80%, at least 85%, at least 90%, at least 95%, or 100% identical to any one of SEQ ID NOs: 773, 775, or 793, and wherein the effector protein is fused to a KRAB domain, a methyltransferase, or a combination thereof.

Embodiment 176: An expression cassette comprising, from 5′ to 3′:
a) a first inverted terminal repeat (ITR);
b) a first promoter sequence operably linked to a nucleic acid sequence encoding a guide RNA wherein the guide RNA comprises:
   i. a first region comprising a protein binding sequence; and
   ii. a second region comprising a spacer sequence that is complementary to a target sequence of an APOC3 gene, wherein the spacer sequence is at least 70%, at least 75%, at least 80%, at least 85%, at least 90%, at least 95% or 100% identical to any one of SEQ ID NOs: 1-15, 67-72, 207, 209-299, 804-805, 823-825, 830-1399, 2018-2026, and 2084-2086;
c) a second promoter sequence operably linked to a nucleic acid sequence encoding an effector protein;
d) a poly(A) signal; and
e) a second ITR.

Embodiment 177: An expression cassette comprising, from 5′ to 3′:
a) a first inverted terminal repeat (ITR);
b) a first promoter sequence operably linked to a nucleic acid sequence encoding a guide RNA wherein the guide RNA comprises:
   iii. a first region comprising a protein binding sequence; and
   iv. a second region comprising a spacer sequence that is complementary to a target sequence of a PCSK9 gene, wherein the spacer sequence is at least 70%, at least 75%, at least 80%, at least 85%, at least 90%, at least 95% or 100% identical to any one of SEQ ID NOs: 79-140, 208, 300-487, 799-803, 809, 822, and 1970-1995;
c) a second promoter sequence operably linked to a nucleic acid sequence encoding an effector protein;
d) a poly(A) signal; and
e) a second ITR.

Embodiment 178: An expression cassette comprising, from 5′ to 3′:
a) a first inverted terminal repeat (ITR);
b) a first promoter sequence operably linked to a nucleic acid sequence encoding a guide RNA wherein the guide RNA comprises:
   v. a first region comprising a protein binding sequence; and
   vi. a second region comprising a spacer sequence that is complementary to a target sequence of a ANGPTL3 gene, wherein the spacer sequence is at least 70%, at least 75%, at least 80%, at least 85%, at least 90%, at least 95% or 100% identical to any one of SEQ ID NOs: 806-808 and 1996-2017;
c) a second promoter sequence operably linked to a nucleic acid sequence encoding an effector protein;
d) a poly(A) signal; and
e) a second ITR.

Embodiment 179: The expression cassette of any of embodiments 176-178, wherein the expression cassette further comprises a WPRE sequence located between the nucleic acid sequence encoding an effector protein and the poly(A) signal.

Embodiment 180: The expression cassette of any of embodiments 176-179, wherein 5 the first promoter is a U6 promoter, the second promoter is a CK8E promoter or a SPC5 promoter or a combination thereof.

Embodiment 181: The expression cassette of any one of embodiments 176-180, wherein the poly(A) signal is a bGH or an hGH poly(A) signal.

Embodiment 182: The expression cassette of any one of embodiments 176-181, wherein
a) the targeting sequence comprises a nucleotide sequence that is at least 70%, at least 75%, at least 80%, at least 85%, at least 90%, at least 95% or 100% identical to any one of SEQ ID NOs: 1-15, 67-72, 207, 804-805, and 830-999, and
b) the effector protein comprises an amino acid sequence that is at least 70%, at least 75%, at least 80%, at least 85%, at least 90%, at least 95% or 100% identical to any one of SEQ ID NOs: 32, 34, 794, or 2090,
c) optionally wherein the protein binding sequence comprises a nucleotide sequence that is at least 70%, at least 75%, at least 80%, at least 85%, at least 90%, at least 95% or 100% identical to any one of SEQ ID NOs: 16 and 38-43.

Embodiment 183: The expression cassette of embodiment 182, wherein the guide RNA comprises a nucleotide sequence that is at least 70%, at least 75%, at least 80%, at least 85%, at least 90%, at least 95% or 100% identical to any one of SEQ ID NOs: 17-31, 73-78, 491, 815-816, and 1400-1569.

Embodiment 184: The expression cassette of any one of embodiments 176-181, wherein
a) the targeting sequence comprises a nucleotide sequence that is at least 70%, at least 75%, at least 80%, at least 85%, at least 90%, at least 95% or 100% identical to any one of SEQ ID NOs: 79-140, 208, 799-803, and 809, and
b) the effector protein comprises an amino acid sequence that is at least 70%, at least 75%, at least 80%, at least 85%, at least 90%, at least 95% or 100% identical to any one of SEQ ID NOs: 32, 34, 794, or 2090,
c) optionally wherein the protein binding sequence comprises a nucleotide sequence that is at least 70%, at least 75%, at least 80%, at least 85%, at least 90%, at least 95% or 100% identical to any one of SEQ ID NOs: 16 and 38-43.

Embodiment 185: The expression cassette of embodiment 184, wherein the guide RNA comprises a nucleotide sequence that is at least 70%, at least 75%, at least 80%, at least 85%, at least 90%, at least 95% or 100% identical to any one of SEQ ID NOs: 141-202, 492-493, 810-814, and 820.

Embodiment 186: The expression cassette of any one of embodiments 176-181, wherein
a) the targeting sequence comprises a nucleotide sequence that is at least 70%, at least 75%, at least 80%, at least 85%, at least 90%, at least 95% or 100% identical to any one of SEQ ID NOs: 806-808, and
b) the effector protein comprises an amino acid sequence that is at least 70%, at least 75%, at least 80%, at least 85%, at least 90%, at least 95% or 100% identical to SEQ ID NOs: 32, 34, 794, or 2090,
c) optionally wherein the protein binding sequence comprises a nucleotide sequence that is at least 70%, at least 75%, at least 80%, at least 85%, at least 90%, at least 95% or 100% identical to any one of SEQ ID NOs: 16 and 38-43.

Embodiment 187: The expression cassette of embodiment 186, wherein the guide RNA comprises a nucleotide sequence that is at least 70%, at least 75%, at least 80%, at least 85%, at least 90%, at least 95% or 100% identical to any one of SEQ ID NOs: 817-819.

Embodiment 188: The expression cassette of any one of embodiments 176-181, wherein
a) the targeting sequence comprises a nucleotide sequence that is at least 70%, at least 75%, at least 80%, at least 85%, at least 90%, at least 95% or 100% identical to any one of SEQ ID NOs: 209-299, 823-825, 1000-1399, 2018-2026, and 2084-2086, and
b) the effector protein comprises an amino acid sequence that is at least 70%, at least 75%, at least 80%, at least 85%, at least 90%, at least 95% or 100% identical to any one of SEQ ID NOs: 773, 775, or 793,
c) optionally wherein the protein binding sequence comprises a nucleotide sequence that is at least 70%, at least 75%, at least 80%, at least 85%, at least 90%, at least 95% or 100% identical to SEQ ID NOs: 488 or 489, or a combination thereof.

Embodiment 189: The expression cassette of embodiment 188, wherein the guide RNA comprises a nucleotide sequence that is at least 70%, at least 75%, at least 80%, at least 85%, at least 90%, at least 95% or 100% identical to any one of SEQ ID NOs: 494-584, 826-828, 1570-1969, 2075-2083, and 2087-2089.

Embodiment 190: The expression cassette of any one of embodiments 176-181, wherein
a) the targeting sequence comprises a nucleotide sequence that is at least 70%, at least 75%, at least 80%, at least 85%, at least 90%, at least 95% or 100% identical to any one of SEQ ID NOs: 300-487, 822, and 1970-1995, and
b) the effector protein comprises an amino acid sequence that is at least 70%, at least 75%, at least 80%, at least 85%, at least 90%, at least 95% or 100% identical to any one of SEQ ID NOs: 773, 775, or 793,
c) optionally wherein the protein binding sequence comprises a nucleotide sequence that is at least 70%, at least 75%, at least 80%, at least 85%, at least 90%, at least 95% or 100% identical to SEQ ID NOs: 488 or 489, or a combination thereof.

Embodiment 191: The expression cassette of embodiment 190, wherein the guide RNA comprises a nucleotide sequence that is at least 70%, at least 75%, at least 80%, at least 85%, at least 90%, at least 95% or 100% identical to any one of SEQ ID NOs: 585-772, 829, and 2027-2052.

Embodiment 192:The expression cassette of any one of embodiments 176-181, wherein
a) the targeting sequence comprises a nucleotide sequence that is at least 70%, at least 75%, at least 80%, at least 85%, at least 90%, at least 95% or 100% identical to any one of SEQ ID NOs: 1996-2017, and
b) the effector protein comprises an amino acid sequence that is at least 70%, at least 75%, at least 80%, at least 85%, at least 90%, at least 95% or 100% identical to any one of SEQ ID NOs: 773, 775, or 793,
c) optionally wherein the protein binding sequence comprises a nucleotide sequence that is at least 70%, at least 75%, at least 80%, at least 85%, at least 90%, at least 95% or 100% identical to SEQ ID NOs: 488 or 489, or a combination thereof.

Embodiment 193: The expression cassette of embodiment 192, wherein the guide RNA comprises a nucleotide sequence that is at least 70%, at least 75%, at least 80%, at least 85%, at least 90%, at least 95% or 100% identical to any one of SEQ ID NOs: 2053-2074.

Embodiment 194: An adeno-associated virus (AAV) vector comprising the expression cassette of any one of embodiments 176-193.

Embodiment: 195: A lipid nanoparticle (LNP) comprising the composition or system of any one of embodiments 145-175, the expression caste of any one of embodiments 176-193, or the AAV vector of embodiment 194.

Embodiment: 196: A pharmaceutical composition comprising the composition or system of any one of embodiments 145-175, the expression caste of any one of embodiments 176-193, or the AAV vector of embodiment 194, and a pharmaceutical acceptable carrier.

Embodiment 197: A cell, or population of cells, comprising the composition or system of any one of embodiments 145-175, the expression caste of any one of embodiments 176-193, the AAV vector of embodiment 194, or the LNP of embodiment 195.

Embodiment 198: A method of modifying an APOC3 gene, comprising contacting the APOC3 gene, with the composition or system of any one embodiments 145-175, the expression caste of any one of embodiments 176-193, the AAV vector of embodiment 194, the LNP of embodiment 195, or the pharmaceutical composition of embodiment 196.

Embodiment 199: A method of modifying a PCSK9 gene, comprising contacting the PCSK9 gene with the composition or system of any one embodiments 145-175, the expression caste of any one of embodiments 176-193, the AAV vector of embodiment 194, the LNP of embodiment 195, or the pharmaceutical composition of embodiment 196.

Embodiment 200: A method of modifying an ANGPTL3 gene, comprising contacting the PCSK9 gene with the composition or system of any one embodiments 145-175, the expression caste of any one of embodiments 176-193, the AAV vector of embodiment 194, the LNP of embodiment 195, or the pharmaceutical composition of embodiment 196.

Embodiment 201: The method of any one of embodiments 198-200, wherein the modifying of the APOC3 gene, the PCSK9 gene, or the ANGPTL3 gene reduces the expression of the APOC3 gene, the PCSK9 gene, or the ANGPTL3 gene.

Embodiment 202: The method of embodiment 201, wherein the reduced expression of the APOC3 gene, the PCSK9 gene, or the ANGPTL3 gene is transient.

Embodiment 203: The method of embodiment 201, wherein the reduced expression of the APOC3 gene, the PCSK9 gene, or the ANGPTL3 gene is permanent.

Embodiment 204: A method of treating or preventing a disease in a subject in need thereof, comprising administering the composition or system of any one of embodiments 145-175, the expression caste of any one of embodiments 176-193, the AAV vector of embodiment 194, the LNP of embodiment 195, or the pharmaceutical composition of embodiment 196, wherein the disease is associated with increased expression of APOC3.

Embodiment 205: A method of treating or preventing a disease in a subject in need thereof, comprising administering the composition or system of any one of embodiments 145-175, the expression caste of any one of embodiments 176-193, the AAV vector of embodiment 194, the LNP of embodiment 195, or the pharmaceutical composition of embodiment 196, wherein the disease is associated with increased expression of PCSK9.

Embodiment 206: A method of treating or preventing a disease in a subject in need thereof, comprising administering the composition or system of any one of embodiments 145-175, the expression caste of any one of embodiments 176-193, the AAV vector of embodiment 194, the LNP of embodiment 195, or the pharmaceutical composition of embodiment 196, wherein the disease is associated with increased expression of ANGPTL3.

Embodiment 207: The method of any one of embodiments 198-206, comprising modifying the APOC3 gene, the PCSK9 gene, or the ANGPTL3 gene in a cell.

Embodiment 208: The method of embodiment 207, wherein the cell is in vivo.

Embodiment 209: The method of any of embodiments 207 or 208, wherein the cell is within a subject having a cardiovascular disease.

Embodiment 210: The method of embodiment 209, wherein the cardiovascular disease is atherosclerotic cardiovascular disease or is coronary artery disease (CAD).

Embodiment 211: The method of any of embodiments 209 or 210, wherein the cell is within a subject having a chronic kidney disease (CKD).

Embodiment 212: The method of any of embodiments 209 or 210, wherein the cell is within a subject having familial chylomicronemia syndrome (FCS).

Embodiment 213: The method of any of embodiments 209 or 210, wherein the cell is within a subject having lipodystrophy.

Embodiment 214: The method of any of embodiments 209 or 210, wherein the cell is within a subject having hypertriglyceridemia.

Embodiment 215: The method of embodiment 214, wherein the disease is severe hypertriglyceridemia.

Embodiment 216: A cell modified by the composition, system, expression cassette, AAV vector, or method of any one of embodiments 145-215.

Embodiment 217: A system comprising a guide ribonucleic acid (RNA) or a polynucleotide encoding the same, wherein the guide RNA comprises:
a) a first region comprising SEQ ID NO: 39, and
b) a second region comprising SEQ ID NO: 10, which is complementary to a target sequence that is within an APOC3 gene,
   wherein the target sequence is adjacent to a protospacer adjacent motif (PAM) 5′-NNTN-3′.

Embodiment 218: The system of embodiment 217, wherein the second region consists of SEQ ID NO: 10.

Embodiment 219: The system of embodiments 217 or 218, wherein the guide RNA comprises the amino acid sequence of SEQ ID NO: 26.

Embodiment 220: The system of embodiment 219, wherein the system further comprises an effector protein, wherein the effector protein comprises the amino acid sequence of SEQ ID NOs: 32, 34, 794, or 2090.

Embodiment 221: A composition comprising a guide ribonucleic acid (RNA) or a polynucleotide encoding the same, wherein the guide RNA comprises:
a) a first region comprising SEQ ID NO: 39, and
b) a second region comprises SEQ ID NO: 10.

Embodiment 222: The composition of embodiment 221, wherein the second region consists of SEQ ID NO: 10.

Embodiment 223: The composition of embodiments 221 or 222, wherein the guide RNA sequence comprises SEQ ID NO: 26.

Embodiment 224: An expression cassette comprising, from 5′ to 3′:
a) a first inverted terminal repeat (ITR);
b) a first promoter sequence operably linked to a nucleic acid sequence encoding a guide ribonucleic acid (RNA) wherein the guide RNA comprises:
   vii. a first region comprising SEQ ID NO: 39; and
   viii. a second region comprising a spacer sequence that is complementary to a target sequence of an APOC3 gene, wherein the spacer sequence comprises SEQ ID NO: 10;
c) a second promoter sequence operably linked to a nucleic acid sequence encoding an effector protein;
d) a poly(A) signal; and
e) a second ITR.

Embodiment 225: The expression cassette of embodiment 224, wherein the second region consists of SEQ ID NO: 10.

Embodiment 226: The expression cassette of any one of embodiments 224 or 225, wherein the guide RNA sequence comprises SEQ ID NO: 26.

Embodiment 227: A recombinant adeno-associated virus (rAAV) expression cassette comprising sequences encoding
a) a first inverted terminal repeat (ITR) and a first promoter;
b) an effector protein that comprises the amino acid sequence of SEQ ID NOs: 32, 34, 794, or 2090;
c) optionally a second promoter;
d) a second polynucleotide encoding a guide ribonucleic acid (RNA), wherein the guide RNA comprises a spacer sequence comprising SEQ ID NO: 10 and a repeat sequence comprising SEQ ID NO: 39; and
e) a second ITR,
wherein the AAV expression cassette is a self-complementary AAV vector.

Embodiment 228: The rAAV expression cassette of embodiment 227, wherein the spacer sequence consists of SEQ ID NO: 10.

Embodiment 229: The rAAV expression cassette of any one of embodiments 227 or 228, wherein the guide RNA sequence comprises SEQ ID NO: 26.

Embodiment 230: A nucleic acid expression vector that encodes a guide ribonucleic acid (RNA), wherein the guide RNA comprises a spacer sequence wherein the spacer sequence comprises SEQ ID NO: 10 and a repeat sequence comprising SEQ ID NO: 39.

Embodiment 231: The nucleic acid expression vector of embodiments 230, wherein the spacer sequence consists of SEQ ID NO: 10.

Embodiment 232: The nucleic acid expression vector of any one of embodiments 230 or 231, where in the guide RNA sequence comprises SEQ ID NO: 26.

Embodiment 233: A guide ribonucleic acid (RNA) or a polynucleotide encoding the same, wherein the guide RNA comprises:
a. a first region comprising SEQ ID NO: 39, and
b. a second region comprises SEQ ID NO: 10.

Embodiment 234: The guide RNA of embodiment 233, wherein the second region consists of SEQ ID NO: 10.

Embodiment 235: The guide RNA of any one of embodiment 233 or 234, wherein the guide RNA comprises SEQ ID NO: 26.

Embodiment 236: A lipid nanoparticle (LNP) comprising the system of embodiments 217-220, the composition of embodiments 221-223, the expression cassette of embodiments 224-226, the rAAV of embodiments 227-229, the nucleic acid expression vector of embodiments 230-232, or the guide RNA of embodiments 233-235.

Embodiment 237: A pharmaceutical comprising the system of embodiments 217-220, the composition of embodiments 221-223, the expression cassette of embodiments 224-226, the rAAV of embodiments 227-229, the nucleic acid expression vector of embodiments 230-232, the guide RNA of embodiments 233-235, or the LNP of embodiment 236, and a pharmaceutically acceptable carrier.

Embodiment 238: A cell modified by the system of embodiments 217-220, the composition of embodiments 221-223, the expression cassette of embodiments 224-226, the rAAV of embodiments 227-229, the nucleic acid expression vector of embodiments 230-232, the guide RNA of embodiments 233-235, or the LNP of embodiment 236.

Embodiment 239: A method of modifying an APOC3 gene, comprising contacting the APOC3 gene with the system of embodiments 217-220, the composition of embodiments 221-223, the expression cassette of embodiments 224-226, the rAAV of embodiments 227-229, the nucleic acid expression vector of embodiments 230-232, the guide RNA of embodiments 233-235, or the LNP of embodiment 236.

Embodiment 240: A method of treating or preventing a disease in a subject in need thereof, comprising administering the system of embodiments 217-220, the composition of embodiments 221-223, the expression cassette of embodiments 224-226, the rAAV of embodiments 227-229, the nucleic acid expression vector of embodiments 230-232, the guide RNA of embodiments 233-235, or the LNP of embodiment 236, or the pharmaceutical composition of embodiment 237, wherein the disease is associated with increased expression of APOC3.

Embodiment 241: The method of any of embodiment 240, wherein the disease is a cardiovascular disease, atherosclerotic cardiovascular disease, coronary artery disease (CAD), a chronic kidney disease (CKD), familial chylomicronemia syndrome (FCS), lipodystrophy, hypertriglyceridemia, or severe hypertriglyceridemia.

Embodiment 242: A system comprising a guide ribonucleic acid (RNA) or a polynucleotide encoding the same, wherein the guide RNA comprises:
f) a first region comprising SEQ ID NO: 39, and
g) a second region comprising SEQ ID NO: 71, which is complementary to a target sequence that is within an APOC3 gene,
   wherein the target sequence is adjacent to a protospacer adjacent motif (PAM) 5′-NNTN-3′.

Embodiment 243: The system of embodiment 242, wherein the second region consists of SEQ ID NO: 71.

Embodiment 244: The system of any one of embodiments 242 or 243, wherein the guide RNA comprises the amino acid sequence of SEQ ID NO: 77.

Embodiment: 245: The system of embodiment 244, wherein the system further comprises an effector protein, wherein the effector protein comprises the amino acid sequence of SEQ ID NOs: 32, 34, 794, or 2090.

Embodiment 246: A composition comprising a guide ribonucleic acid (RNA) or a polynucleotide encoding the same, wherein the guide RNA comprises:
h) a first region comprising SEQ ID NO: 39, and
i) a second region comprising SEQ ID NO: 71.

Embodiment 247: The composition of embodiment 246, wherein the second region consists of SEQ ID NO: 71.

Embodiment 248: The composition of any one of embodiments 246 or 247, wherein the guide RNA sequence comprises SEQ ID NO: 77.

Embodiment 249: An expression cassette comprising, from 5′ to 3′:
j) a first inverted terminal repeat (ITR);
k) a first promoter sequence operably linked to a nucleic acid sequence encoding a guide ribonucleic acid (RNA) wherein the guide RNA comprises:
   ix. a first region comprising SEQ ID NO: 39; and
   x. a second region comprising a spacer sequence that is complementary to a target sequence of an APOC3 gene, wherein the spacer sequence comprising SEQ ID NO: 71;
l) a second promoter sequence operably linked to a nucleic acid sequence encoding an effector protein;
m) a poly(A) signal; and
n) a second ITR.

Embodiment 250: The expression cassette of embodiment 249, wherein the second region consists of SEQ ID NO: 71.

Embodiment 251: The expression cassette of any one of embodiments 249 or 250, wherein the guide RNA sequence comprises SEQ ID NO: 77.

Embodiment 252: A recombinant adeno-associated virus (rAAV) expression cassette comprising sequences encoding
o) a first inverted terminal repeat (ITR) and a first promoter;
p) an effector protein that comprises the amino acid sequence of SEQ ID NOs: 32, 34, 794, or 2090;
q) optionally a second promoter;
r) a second polynucleotide encoding a guide ribonucleic acid (RNA), wherein the guide RNA comprises a spacer sequence comprising SEQ ID NO: 71 and a repeat sequence comprising SEQ ID NO: 39; and
s) a second ITR,
wherein the AAV expression cassette is a self-complementary AAV vector.

Embodiment 253: The rAAV expression cassette of embodiment 252, wherein the spacer sequence consists of SEQ ID NO: 77.

Embodiment 254: The rAAV expression cassette of any one of embodiments 252 or 253, wherein the guide RNA sequence comprises SEQ ID NO: 77.

Embodiment 255: A nucleic acid expression vector that encodes a guide ribonucleic acid (RNA), wherein the guide RNA comprises a spacer sequence wherein the spacer sequence comprising SEQ ID NO: 71 and a repeat sequence comprising SEQ ID NO: 39.

Embodiment 256: The nucleic acid expression vector of embodiment 255, wherein the spacer sequence consists of SEQ ID NO: 71.

Embodiment 257: The nucleic acid expression vector of any one of embodiments 255 or 256, where in the guide RNA sequence comprises SEQ ID NO: 77.

Embodiment 258: A guide ribonucleic acid (RNA) or a polynucleotide encoding the same, wherein the guide RNA comprises:
a. a first region comprising SEQ ID NO: 39, and
b. a second region comprising SEQ ID NO: 71.

Embodiment 259: The guide RNA of embodiment 258, wherein the second region consists of SEQ ID NO: 71.

Embodiment 260: The guide RNA of any one of embodiments 258 or 259, wherein the guide RNA comprises SEQ ID NO: 77.

Embodiment 261: A lipid nanoparticle (LNP) comprising the system of any one of embodiments 242-245, the composition of embodiments 246-248, the expression cassette of embodiments 249-251, the rAAV of embodiments 252-254, the nucleic acid expression vector of embodiments 255-257, or the guide RNA of embodiments 258-260.

Embodiment 262: A pharmaceutical comprising the of any one of embodiments 242-245, the composition of embodiments 246-248, the expression cassette of embodiments 249-251, the rAAV of embodiments 252-254, the nucleic acid expression vector of embodiments 255-257, the guide RNA of embodiments 258-260, or the LNP of embodiment 261, and a pharmaceutically acceptable carrier.

Embodiment 263: A cell modified by the system of any one of embodiments 242-245, the composition of embodiments 246-248, the expression cassette of embodiments 249-251, the rAAV of embodiments 252-254, the nucleic acid expression vector of embodiments 255-257, the guide RNA of embodiments 258-260, or the LNP of embodiment 261.

Embodiment 264: A method of modifying an APOC3 gene, comprising contacting the APOC3 gene with the system of any one of embodiments 242-245, the composition of embodiments 246-248, the expression cassette of embodiments 249-251, the rAAV of embodiments 252-254, the nucleic acid expression vector of embodiments 255-257, the guide RNA of embodiments 258-260, or the LNP of embodiment 261.

Embodiment 265: A method of treating or preventing a disease in a subject in need thereof, comprising administering the system of any one of embodiments 242-245, the composition of embodiments 246-248, the expression cassette of embodiments 249-251, the rAAV of embodiments 252-254, the nucleic acid expression vector of embodiments 255-257, the guide RNA of embodiments 258-260, or the LNP of embodiment 261, wherein the disease is associated with increased expression of APOC3.

Embodiment 266: The method of embodiment 265, wherein the disease is a cardiovascular disease, atherosclerotic cardiovascular disease, coronary artery disease (CAD), a chronic kidney disease (CKD), familial chylomicronemia syndrome (FCS), lipodystrophy, hypertriglyceridemia, or severe hypertriglyceridemia.

### EXAMPLES

The following examples are included for illustrative purposes only and are not intended to limit the scope of the invention.

### Example 1: CasPhi.12 Modifies APOC3

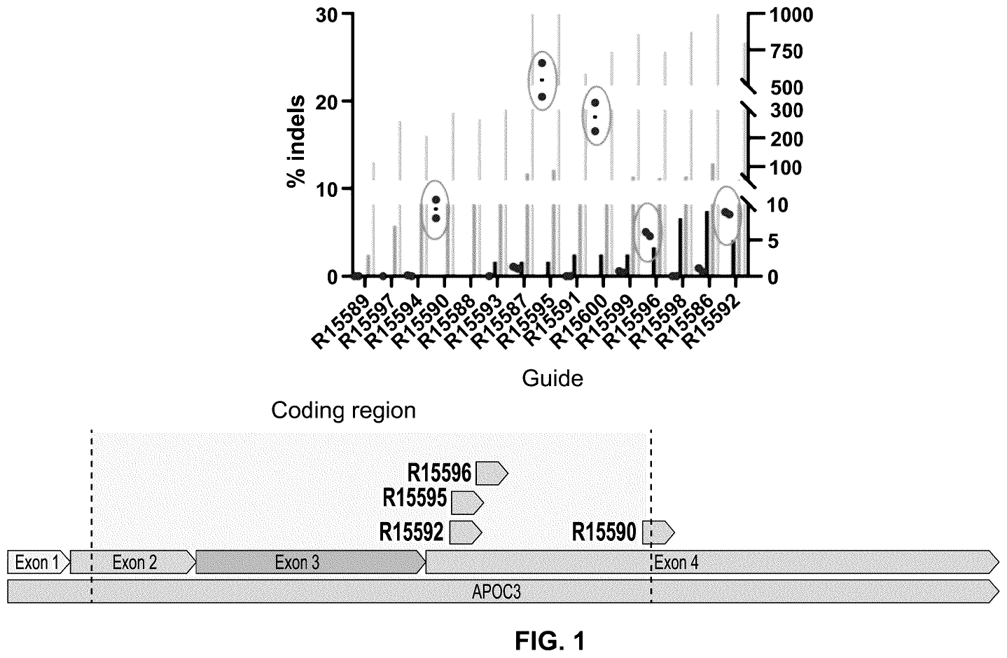

Mammalian (Macaca fascicularis) skin fibroblasts (CYNOM-K1 Cells) were transfected with plasmids encoding CasPhi.12 L26R and guides targeting APOC3 using Messenger Max in a 96-well format, with a 1:1 mRNA to gRNA ratio (100 ng mRNA+100 ng gRNA=200 ng Total RNA). Guides were human monkey cross-reactive. Results are provided in FIG. 1. FIG. 1 also shows where tested guides target the APOC3 gene.

### Example 2: APOC3 Editing Systems Achieved Greater than 40% Indels and APOC3 Protein Reduction in HepG2 Cells

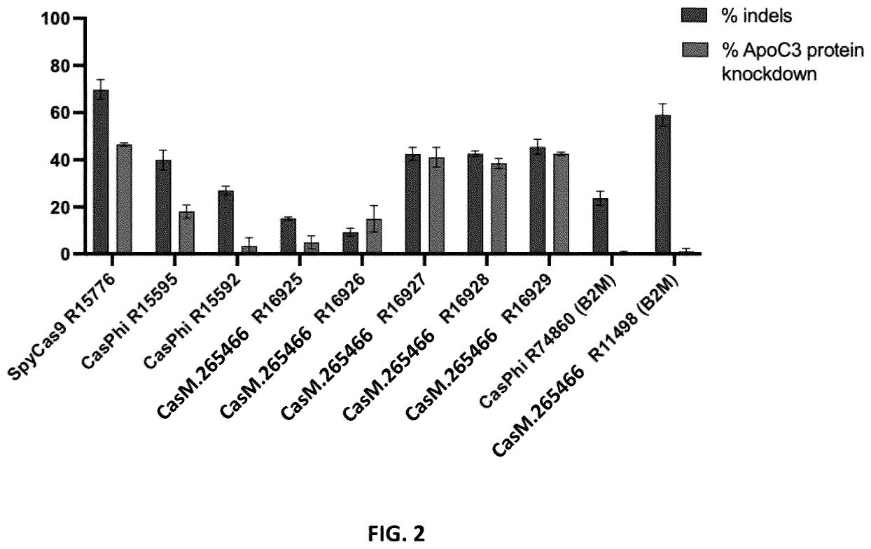

HepG2 cells were transfected (MessengerMax) with CasPhi.12 L26R mRNA or CasM.265466 D220R mRNA and various guides targeting human APOC3 gene. SpyCas9 was included as a control. After 5 days, cells were harvested and indels were quantified by NGS. APOC3 protein was quantified by ELISA. Results are provided in FIG. 2. For each guide, the column on the left is the percent indel formation and the column on the right is the percent APOC3 protein knockdown.

### Example 3: Indel Activity of Effector Protein/Guide RNA on APOC3 in Cynomolgus Hepatocytes

Experiments were performed to assess the efficacy of different APOC3-targeting gRNAs. Briefly, 100,000 primary cynomolgus hepatocytes were transfected with CasPhi.12 L26R mRNA and gRNA (1:1 ratio) combinations at both 200 ng and 50 ng of guide RNA using MessengerMax while rocking in 96-well low attachment plates for 2 hours. Hepatocytes were then transferred to 96-well Collagen I coated plates and cultured for 48 hours, followed by MTS assay and NGS. The gRNAs tested in this assay are as follows: R15590 (SEQ ID NO: 21), R15592 (SEQ ID NO: 23), R15595 (SEQ ID NO: 26), 15596 (SEQ ID NO: 27), and 15600 (SEQ ID NO: 31). Cell viability was not affected by transfection.

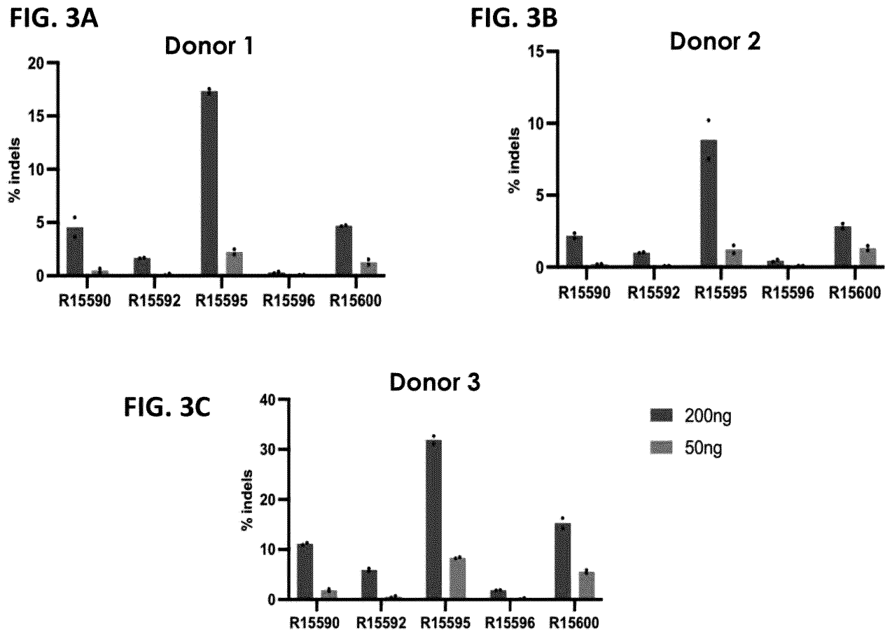

FIGS. 3A-3C show the percent indel formation in three different donors. R15595 had an at least 10% indel formation efficiency in the hepatocytes from all three donors. For each guide, the column on the left is the percent indel formation with 200 ng of the guide RNA and the column on right is the percent indel formation with 50 ng of the guide RNA.

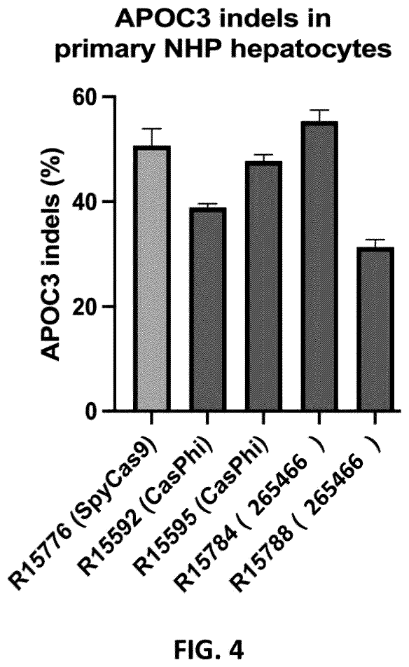

FIG. 4 shows that (in a separate but similarly performed experiment) increasing the amount of guide RNA transfected (500 ng) and length of incubation (5 days) leads to a concomitant increase in the percent indel formation. Similar levels of editing were obtained with CasM.265466 D220R and guides (R15784 (SEQ ID NO: 583) and R15788 (SEQ ID NO: 584)), also shown in FIG. 4.

### Example 4: Additional APOC3 Guides for CasPhi.12 and CasM.265466 Edit APOC3 in HepG2 Cells

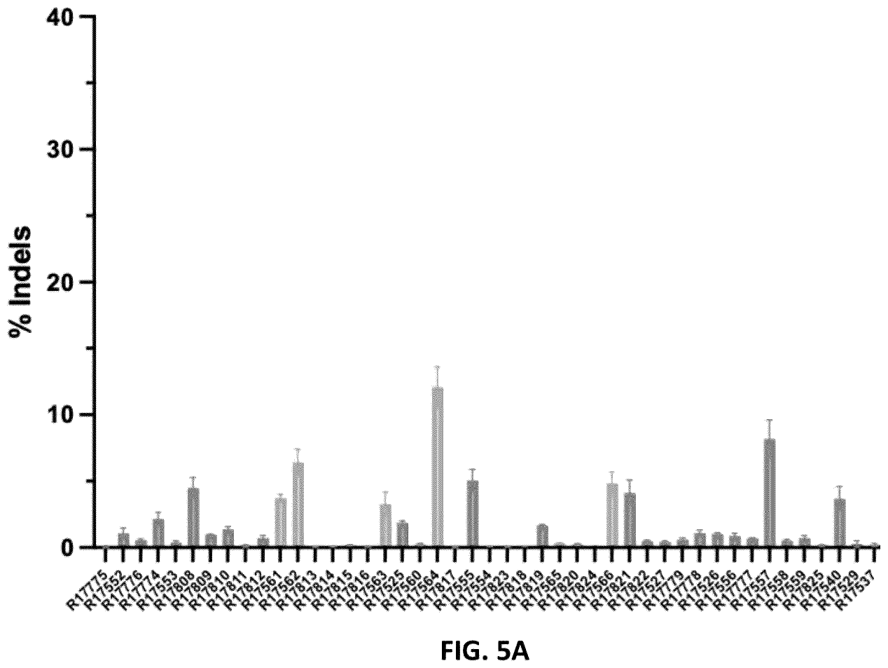

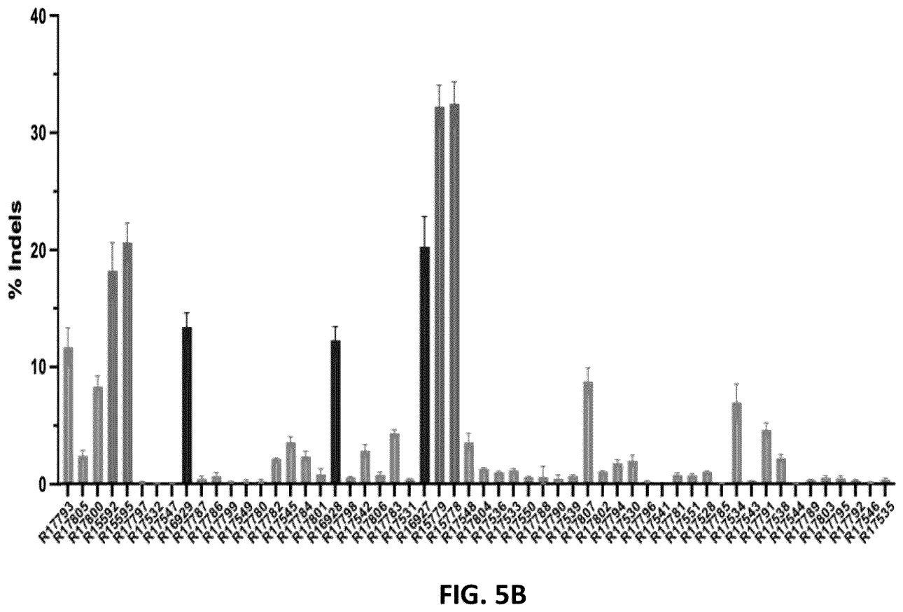

CasPhi.12 L26R and CasM.265466 D220R were tested with additional APOC3 guide nucleic acids for editing of APOC3 in human cells. HepG2 cells were transfected using MessengerMax with 400 ng RNA in a 1:1 guide:mRNA ratio in 96 well scale. SpyCas9 was used as a control. Cells were harvested after 48 hours and indels quantified via NGS. Guide nucleic acids used with CasM.265466 were designed to hybridize near a PAM of NNTN. Results are provided in FIG. 5A-FIG. 5B. Guides R15579 and R15578 were paired with SpyCas9; guides R17561, R17562, R17563, R17564, R17565, R17566, R15592, and R15595 were paired with CasPhi.12; and the rest of the guides were paired with CasM.265466.

### Example 5: APOC3 Editing Systems Provide APOC3 Protein Reduction in HepG2 Cells

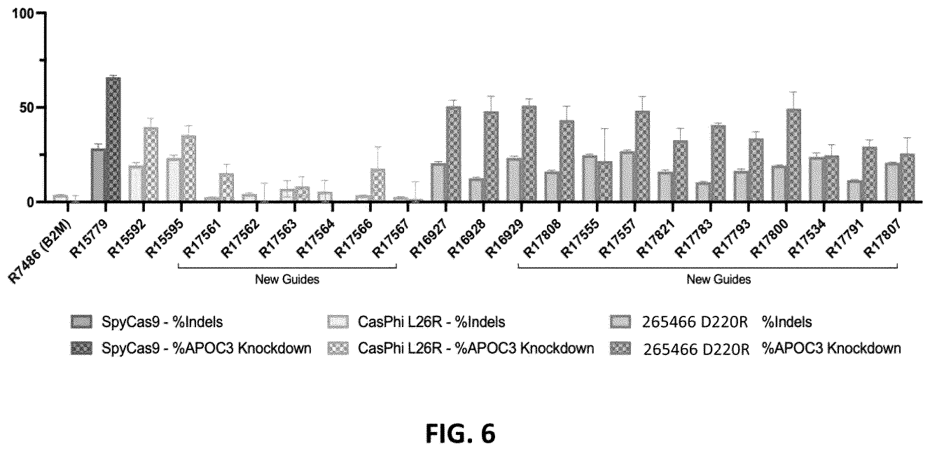

Guide nucleic acids that provided higher levels of APOC3 indels in Example 4 were transfected with CasPhi.12 L26R or CasM.265466 D220R into HepG2 cells with corresponding nucleases to assess APOC3 protein reduction. HepG2 cells were transfected via MessengerMax with 400 ng RNA in a 1:1 guide:mRNA ratio in 96 well scale. NGS and ELISA were performed after 5 days. Results are provided in FIG. 6. Guide R15579 was paired with SpyCas9; guides R15592, R15595, R17561, R17562, R17563, R17564, R17566, and R17567 were paired with CasPhi.12; and the rest of the guides were paired with CasM.265466.

### Example 6: CasPhi.12 and APOC3 Guides Demonstrate APOC3 Editing Across Multiple Mammalian Cell Lines

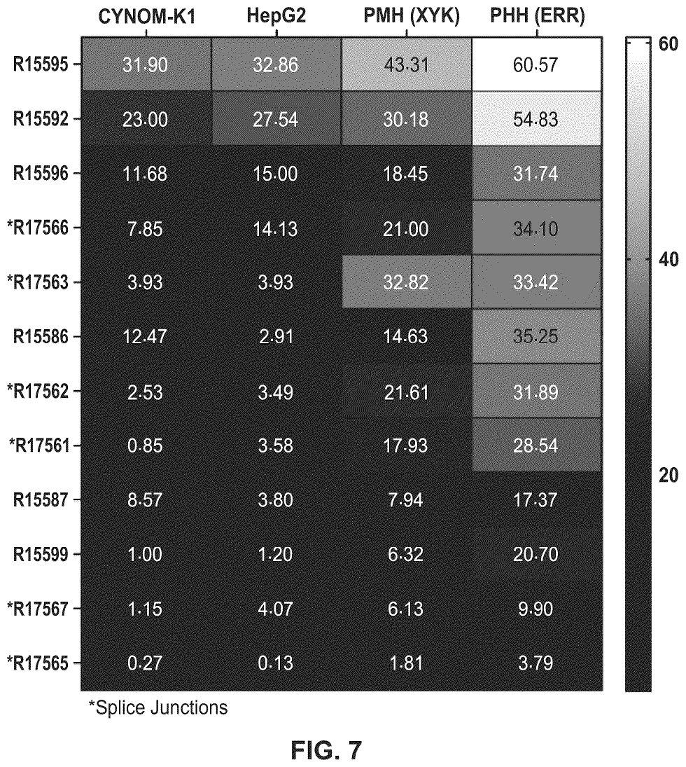

HepG2 cells (20,000 cells/well) were transfected with 400 ng CasPhi.12 L26R mRNA/gRNA (1:1 ratio) using 0.4 uL MessengerMax in a 96-well plate. CYNOM-K1 cells (20,000 cells/well) were transfected with 200 ng CasPhi.12 L26R mRNA/gRNA (1:1 ratio) using 0.2 uL MessengerMax in a 96-well plate. Primary human and monkey hepatocytes (500,000 cells/well) were transfected with 400 ng CasPhi L26R mRNA/gRNA (1:1 ratio) using 0.6 μL MessengerMax in a 96-well plate. Cells were harvested after 48 hours and analyzed by NGS. All guides demonstrated consistent and translatable levels of editing across four human and NHP cell types. Results are shown in FIG. 7, wherein lighter color in the grey-scale heat map is indicative of indel formation.

### Example 7: CasPhi.12 and CasM.265466 Edit APOC3 in Fibroblasts of hAPOC3 Transgenic Mice

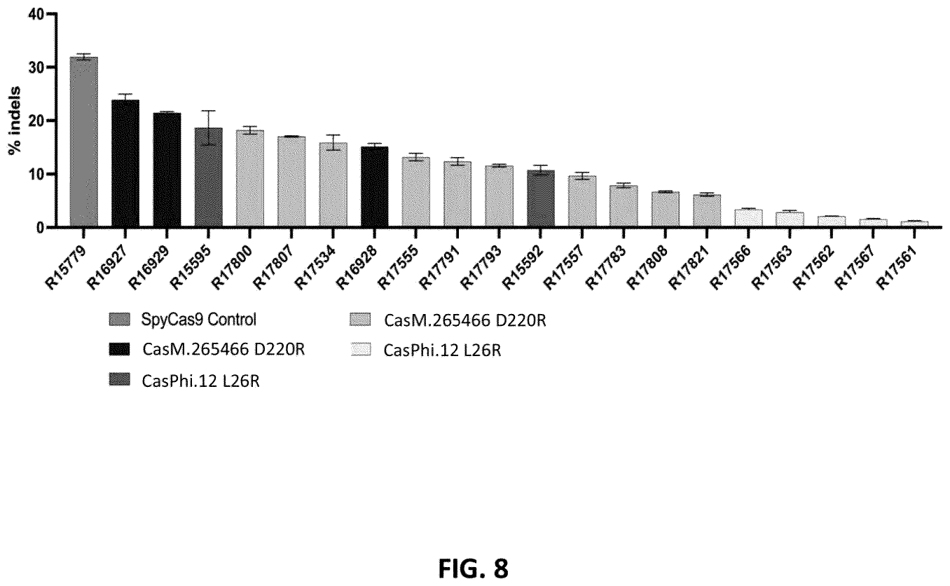

hAPOC3 Mouse fibroblast cells (20,000 cells/well) were transfected with 200 ng mRNA (CasPhi.12 L26R or CasM.265466 D220R) and gRNA at a 1:1 ratio using 0.6 uL MessengerMax in a 96-well plate. Cells were harvested after 48 hours and analyzed by NGS. Results are shown in FIG. 8. Guide R15579 was paired with SpyCas9; guides R15592, R15595, R17561, R17562, R17563, R17566, and R17567 were paired with CasPhi.12; and the rest of the guides were paired with CasM.265466.

### Example 8: Testing Indel Formation of CasPhi.12 Variant in Mice

WT C57BL/6J male mice (n=3-5), aged 6-8 weeks, were injected IV via tail vein with 2 mg/kg of mRNA encoding nuclease and R8860 guide (1:3 ratio) formulated with CKK-E12. Cas9 mRNA and Pcsk9 guide R8217 were also injected as a control. The study was repeated, each study ended 2-7 days post injection and liver was collected for indel analysis by NGS. Data representative of multiple experiments.

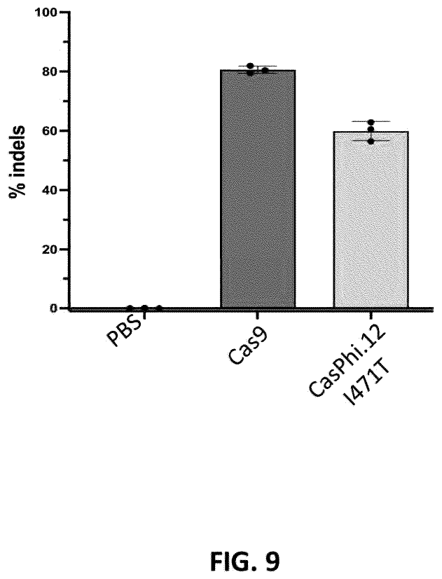

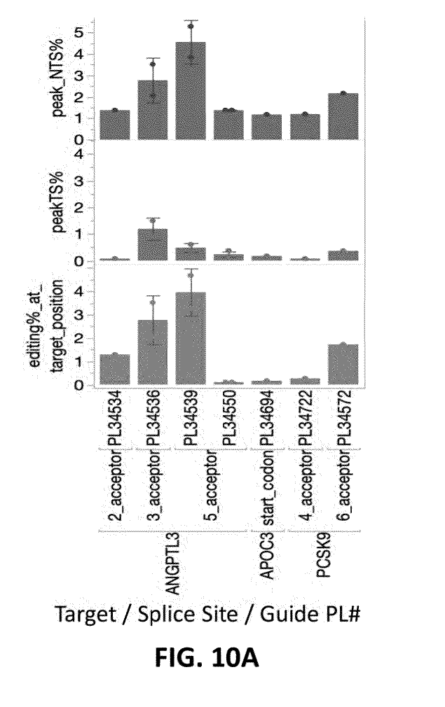

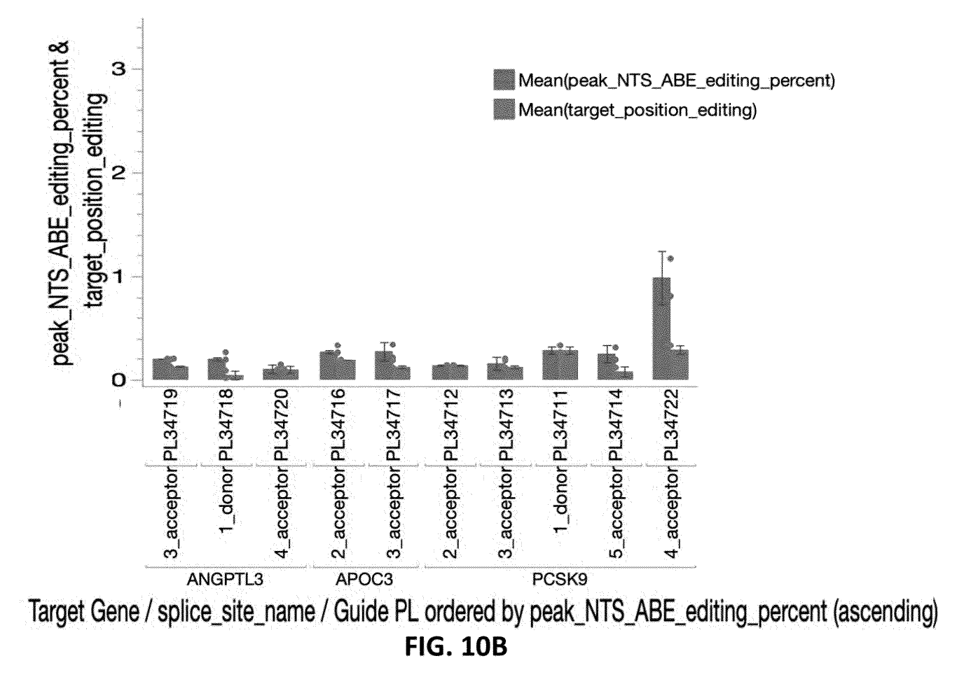

The results demonstrate that CasPhi.12 1471T produces indels comparable with Cas9 in WT mice in repeat studies with different mRNA lots and formulation runs. The sequences of the gRNAs used in this experiment are provided in TABLE 30. Results are shown in FIG. 9. This example demonstrates a CasPhi.12 variant can be delivered via LNP to a mammal in order to edit a gene in liver tissue.

TABLE 30
Exemplary gRNA sequences

| | | Spacer | PAM |
|---|---|---|---|
| Target Locus | Nuclease | sequence | sequence |
| PCSK9 | SaCas9 | | NNGRRT |
| PCSK9 | CasPhi.12 | GAGCAACGG | TTN (TTG) |
| | 1471T | CGGAAGGU | |
| | (SEQ ID NO: | (SEQ ID | |
| | 2091) | NO: 208) | |

The gRNA for CasPhi.12 comprises a repeat sequence
of AUAGAUUGCUCCUUACGAGGAGAC (SEQ ID NO: 39) and
the full sequence of the guide is mA*mU*mA*GA
UUGCUCCUUACGAGGAGACGAGCAACGGCGGAAmG*mG*mU
(SEQ ID NO: 493)

### Example 9: Modifying Nucleobases of PCSK9, APOC3 and ANGPTL3 in Mammalian Cells with Engineered Variants of CasM.265466 and CasPhi.12

Briefly, HEK293T cells were transfected with plasmids encoding a base editor fusion protein and guide nucleic acids (150 ng of dCas466-ABE8e fusion plasmid, 150 ng of guide plasmid). Cells were harvested 72 hours later for analysis via NGS.

The following Effector-base editor fusion proteins were tested: (a) CasM.265466 D220R/E335Q-ABE8e (SEQ ID NO: 797); and (b) CasPhi.12 L26K/E567Q-ABE8e (SEQ ID NO: 798).

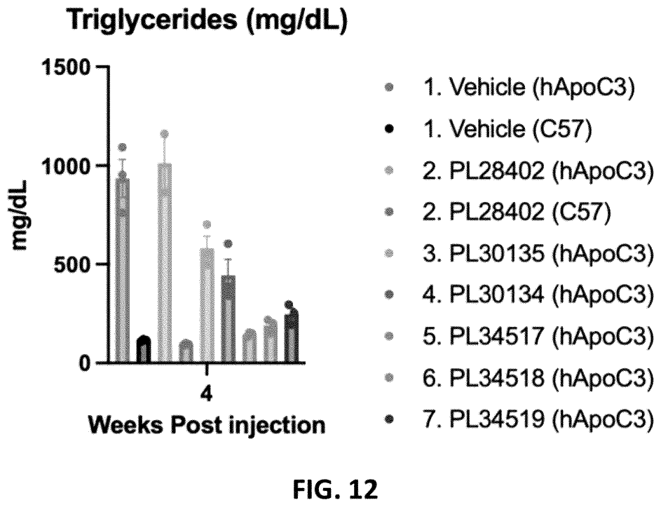

TABLE 31 and TABLE 32 show the spacers and guide nucleic acids that were tested, respectively. Results for CasM.265466 D220R/E335Q are provided in FIG. 9A. Results for CasPhi.12 L26K/E567Q are provided in FIG. 12B. The bar to the left represents mean non-target strand ABE editing percent, and the bar to the right represents mean target position editing.

TABLE 31
Spacer RNAs to be tested

| Intended | Internal | Target | | | SEQ |
|---|---|---|---|---|---|
| Cas* | Ref: | Locus | PAM | Spacer sequence | ID NO: |
| CasPhi.12 | PL34711 | PCSK9 | CTTC | ACCCACCUGUGCCGCGGCGA | 799 |
| CasPhi.12 | PL34712 | PCSK9 | CTTG | CAUGGGGCCAGGAUCCGUGG | 800 |
| CasPhi.12 | PL34713 | PCSK9 | CTTC | UGCAGGCCUUGAAGUUGCCC | 801 |
| CasPhi.12 | PL34714 | PCSK9 | GTTC | GUCGAGCAGGCCAGCAAGUG | 802 |
| CasPhi. 12 | PL34715 | PCSK9 | GTTC | CUCCCAGGCCUGGAGUUUAU | 803 |
| CasPhi.12 | PL34716 | APOC3 | CTTC | CUUGCAGGAACAGAGGUGCC | 804 |
| CasPhi.12 | PL34717 | APOC3 | CTTT | CCUCAGGAGCUUCAGAGGCC | 805 |
| CasPhi.12 | PL34718 | ANGPTL3 | TTTC | UACUUACUUUAAGUGAAGUU | 806 |
| CasPhi.12 | PL34719 | ANGPTL3 | CTTT | UAUCAGCUCAGAAGGACUAG | 807 |
| CasPhi.12 | PL34720 | ANGPTL3 | ATTG | AUUCUAGGCAUUCCUGCUGA | 808 |
| CasPhi.12 | PL34722 | PCSK9 | CTTG | GAAAGACGGAGGCAGCCUGG | 809 |
| CasM.265466 | PL34563 | PCSK9 | TCTA | CACCCGCACCUUGGCGCAGC | 1970 |
| CasM.265466 | PL34564 | PCSK9 | TTTA | GGGCCAGGAUCCGUGGAGGU | 1971 |
| CasM.265466 | PL34565 | PCSK9 | TATA | GCUCACCAGCUCCAGCAGGU | 1972 |
| CasM.265466 | PL34566 | PCSK9 | ATTA | GCUUCUGCAGGCCUUGAAGU | 1973 |
| CasM.265466 | PL34567 | PCSK9 | TTTA | GGGGUCUUACCGGGGGGCUG | 1974 |
| CasM.265466 | PL34568 | PCSK9 | AGTG | GAAAGACGGAGGCAGCCUGG | 1975 |
| CasM.265466 | PL34569 | PCSK9 | TTTA | CUUACCUGUCUGUGGAAGCG | 1976 |
| CasM.265466 | PL34570 | PCSK9 | TATA | UUCGUCGAGCAGGCCAGCAA | 1977 |
| CasM.265466 | PL34571 | PCSK9 | TGTA | GGGCCAUCACUUACCUAUGA | 1978 |
| CasM.265466 | PL34572 | PCSK9 | TTTA | UUCCUCCCAGGCCUGGAGUU | 1979 |
| CasM.265466 | PL34573 | PCSK9 | GGTA | AUGACCUGGAAAGGUGAGGA | 1980 |
| CasM.265466 | PL34574 | PCSK9 | TCTA | CACCAGGCAUUGCAGCCAUG | 1981 |
| CasM.265466 | PL34575 | PCSK9 | ATTA | CUUACCUGCCCCAUGGGUGC | 1982 |
| CasM.265466 | PL34576 | PCSK9 | AATA | CAGUCACCUCCAUGCGCUCG | 1983 |
| CasM.265466 | PL34577 | PCSK9 | CTTG | ACUCUAAGGCCCAAGGGGGC | 1984 |
| CasM.265466 | PL34578 | PCSK9 | AATA | CCCCAGGCUGCAGCUCCCAC | 1985 |
| CasM.265466 | PL34579 | PCSK9 | GGTA | GCAGGUGACCGUGGCCUGCG | 1986 |
| CasM.265466 | PL34580 | PCSK9 | AATG | CCUCGCCGCGGCACAGGUGG | 1987 |
| CasM.265466 | PL34581 | PCSK9 | GTTG | CCAGGCAACCUCCACGGAUC | 1988 |
| CasM.265466 | PL34582 | PCSK9 | TATG | GCGACCUGCUGGAGCUGGUG | 1989 |
| CasM.265466 | PL34583 | PCSK9 | TCTA | AGUGGCGACCUGCUGGAGCU | 1990 |
| CasM.265466 | PL34584 | PCSK9 | ACTG | ACUGUCACACUUGCUGGCCU | 1991 |
| CasM.265466 | PL34585 | PCSK9 | AGTG | CUCCCCAGCCUCAGCUCCCG | 1992 |
| CasM.265466 | PL34586 | PCSK9 | CCTG | GCCCCAACUGUGAUGACCUG | 1993 |
| CasM.265466 | PL34587 | PCSK9 | ACTG | CCCCCCAGCACCCAUGGGGC | 1994 |
| CasM.265466 | PL34588 | PCSK9 | CCTG | CAAAACAGCUGCCAACCUGC | 1995 |
| CasM.265466 | PL34532 | ANGPTL3 | GTTG | CUUACUUUAAGUGAAGUUAC | 1996 |
| CasM.265466 | PL34533 | ANGPTL3 | CCTA | UUUUCUACUUACUUUAAGUG | 1997 |
| CasM.265466 | PL34534 | ANGPTL3 | GCTG | UCCAGACUUUUGUAGAAAAA | 1998 |
| CasM.265466 | PL34535 | ANGPTL3 | CCTG | AAAUACUGACUUACCUGAUU | 1999 |
| CasM.265466 | PL34536 | ANGPTL3 | ACTG | UCAGCUCAGAAGGACUAGUA | 2000 |
| CasM.265466 | PL34537 | ANGPTL3 | CCTA | UCUUACCAUCAUGUUUUACA | 2001 |
| CasM.265466 | PL34538 | ANGPTL3 | CATG | UUGAUUCUAGGCAUUCCUGC | 2002 |
| CasM.265466 | PL34539 | ANGPTL3 | GGTG | UUCAGGUAGUCCAUGGACAU | 2003 |
| CasM.265466 | PL34540 | ANGPTL3 | TCTG | GUCCCCUUACCAUCAAGCCU | 2004 |
| CasM.265466 | PL34541 | ANGPTL3 | GATG | AAACUUUUCUUUUCAGGAGA | 2005 |
| CasM.265466 | PL34542 | ANGPTL3 | CTTG | UCAGAAAAAGAUACCUGAAU | 2006 |
| CasM.265466 | PL34543 | ANGPTL3 | CGTG | UCUCCUUUAGGAGGCUGGUG | 2007 |
| CasM.265466 | PL34544 | ANGPTL3 | TGTG | UCUUGUUUUUCUACAAAAGU | 2008 |
| CasM.265466 | PL34545 | ANGPTL3 | TCTG | AAAGAAAUAGAAAAUCAGGU | 2009 |
| CasM.265466 | PL34546 | ANGPTL3 | TTTG | AAUACUAGUCCUUCUGAGCU | 2010 |
| CasM.265466 | PL34547 | ANGPTL3 | TGTG | AGAAAUGUAAAACAUGAUGG | 2011 |
| CasM.265466 | PL34548 | ANGPTL3 | CCTG | CAUUCAGCAGGAAUGCCUAG | 2012 |
| CasM.265466 | PL34549 | ANGPTL3 | CCTG | GUGGUACAUUCAGCAGGAAU | 2013 |
| CasM.265466 | PL34550 | ANGPTL3 | GGTA | AAUUAAUGUCCAUGGACUAC | 2014 |
| CasM.265466 | PL34551 | ANGPTL3 | TTTG | GUUUUGGGAGGCUUGAUGGU | 2015 |
| CasM.265466 | PL34552 | ANGPTL3 | TCTG | GGCCCAACCAAAAUUCUCCU | 2016 |
| CasM.265466 | PL34553 | ANGPTL3 | TCTG | UCCAGAGGGUUAUUCAGGUA | 2017 |
| CasM.265466 | PL34554 | APOC3 | TCTG | CUUACGGGCAGAGGCCAGGA | 2018 |
| CasM.265466 | PL34555 | APOC3 | GGTG | CUCUUUCCUCAGGAGCUUCA | 2019 |
| CasM.265466 | PL34556 | APOC3 | AGTG | AUUUAGGGGCUGGGUGACCG | 2020 |
| CasM.265466 | PL34557 | APOC3 | GATG | ACUGAUUUAGGGGCUGGGUG | 2021 |
| CasM.265466 | PL34558 | APOC3 | CATG | CUUCCCCUGACUGAUUUAGG | 2022 |
| CasM.265466 | PL34559 | APOC3 | TCTA | GAGGCAGCUGCUCCAGGUAA | 2023 |
| CasM.265466 | PL34560 | APOC3 | GGTG | CAUGGCACCUCUGUUCCUGC | 2024 |
| CasM.265466 | PL34561 | APOC3 | CCTG | GCGCUCCUGGCCUCUGCCCG | 2025 |
| CasM.265466 | PL34562 | APOC3 | CCTG | AAGCCAUCGGUCACCCAGCC | 2026 |

TABLE 32
Full guide RNAs to be tested

| Intended | Internal | Target | | SEQ |
|---|---|---|---|---|
| Cas* | Ref: | Locus | Full Guide Sequence | ID NO: |
| CasPhi.12 | PL34711 | PCSK9 | AUUGCUCCUUACGAGGAGACACC | 810 |
| | | | CACCUGUGCCGCGGCGA | |
| CasPhi.12 | PL34712 | PCSK9 | AUUGCUCCUUACGAGGAGACCAU | 811 |
| | | | GGGGCCAGGAUCCGUGG | |
| CasPhi.12 | PL34713 | PCSK9 | AUUGCUCCUUACGAGGAGACUGC | 812 |
| | | | AGGCCUUGAAGUUGCCC | |
| CasPhi.12 | PL34714 | PCSK9 | AUUGCUCCUUACGAGGAGACGUC | 813 |
| | | | GAGCAGGCCAGCAAGUG | |
| CasPhi.12 | PL34715 | PCSK9 | AUUGCUCCUUACGAGGAGACCUC | 814 |
| | | | CCAGGCCUGGAGUUUAU | |
| CasPhi.12 | PL34716 | APOC3 | AUUGCUCCUUACGAGGAGACCUU | 815 |
| | | | GCAGGAACAGAGGUGCC | |
| CasPhi.12 | PL34717 | APOC3 | AUUGCUCCUUACGAGGAGACCCU | 816 |
| | | | CAGGAGCUUCAGAGGCC | |
| CasPhi.12 | PL34718 | ANGPTL3 | AUUGCUCCUUACGAGGAGACUAC | 817 |
| | | | UUACUUUAAGUGAAGUU | |
| CasPhi.12 | PL34719 | ANGPTL3 | AUUGCUCCUUACGAGGAGACUAU | 818 |
| | | | CAGCUCAGAAGGACUAG | |
| CasPhi.12 | PL34720 | ANGPTL3 | AUUGCUCCUUACGAGGAGACAUU | 819 |
| | | | CUAGGCAUUCCUGCUGA | |
| CasPhi.12 | PL34722 | PCSK9 | AUUGCUCCUUACGAGGAGACGAA | 820 |
| | | | AGACGGAGGCAGCCUGG | |
| CasM.265466 | PL34563 | PCSK9 | ACAGCUUAUUUGGAAGCUGAAAU | 2027 |
| | | | GUGAGGUUUAUAACACUCACAAG | |
| | | | AAUCCUGAAAAAGGAUGCCAAAC | |
| | | | CUUACUUUAAGUGAAGUUAC | |
| CasM.265466 | PL34564 | PCSK9 | ACAGCUUAUUUGGAAGCUGAAAU | 2028 |
| | | | GUGAGGUUUAUAACACUCACAAG | |
| | | | AAUCCUGAAAAAGGAUGCCAAAC | |
| | | | UUUUCUACUUACUUUAAGUG | |
| CasM.265466 | PL34565 | PCSK9 | ACAGCUUAUUUGGAAGCUGAAAU | 2029 |
| | | | GUGAGGUUUAUAACACUCACAAG | |
| | | | AAUCCUGAAAAAGGAUGCCAAAC | |
| | | | UCCAGACUUUUGUAGAAAAA | |
| CasM.265466 | PL34566 | PCSK9 | ACAGCUUAUUUGGAAGCUGAAAU | 2030 |
| | | | GUGAGGUUUAUAACACUCACAAG | |
| | | | AAUCCUGAAAAAGGAUGCCAAAC | |
| | | | AAAUACUGACUUACCUGAUU | |
| CasM.265466 | PL34567 | PCSK9 | ACAGCUUAUUUGGAAGCUGAAAU | 2031 |
| | | | GUGAGGUUUAUAACACUCACAAG | |
| | | | AAUCCUGAAAAAGGAUGCCAAAC | |
| | | | UCAGCUCAGAAGGACUAGUA | |
| CasM.265466 | PL34568 | PCSK9 | ACAGCUUAUUUGGAAGCUGAAAU | 2032 |
| | | | GUGAGGUUUAUAACACUCACAAG | |
| | | | AAUCCUGAAAAAGGAUGCCAAAC | |
| | | | UCUUACCAUCAUGUUUUACA | |
| CasM.265466 | PL34569 | PCSK9 | ACAGCUUAUUUGGAAGCUGAAAU | 2033 |
| | | | GUGAGGUUUAUAACACUCACAAG | |
| | | | AAUCCUGAAAAAGGAUGCCAAAC | |
| | | | UUGAUUCUAGGCAUUCCUGC | |
| CasM.265466 | PL34570 | PCSK9 | ACAGCUUAUUUGGAAGCUGAAAU | 2034 |
| | | | GUGAGGUUUAUAACACUCACAAG | |
| | | | AAUCCUGAAAAAGGAUGCCAAAC | |
| | | | UUCAGGUAGUCCAUGGACAU | |
| CasM.265466 | PL34571 | PCSK9 | ACAGCUUAUUUGGAAGCUGAAAU | 2035 |
| | | | GUGAGGUUUAUAACACUCACAAG | |
| | | | AAUCCUGAAAAAGGAUGCCAAAC | |
| | | | GUCCCCUUACCAUCAAGCCU | |
| CasM.265466 | PL34572 | PCSK9 | ACAGCUUAUUUGGAAGCUGAAAU | 2036 |
| | | | GUGAGGUUUAUAACACUCACAAG | |
| | | | AAUCCUGAAAAAGGAUGCCAAAC | |
| | | | AAACUUUUCUUUUCAGGAGA | |
| CasM.265466 | PL34573 | PCSK9 | ACAGCUUAUUUGGAAGCUGAAAU | 2037 |
| | | | GUGAGGUUUAUAACACUCACAAG | |
| | | | AAUCCUGAAAAAGGAUGCCAAAC | |
| | | | UCAGAAAAAGAUACCUGAAU | |
| CasM.265466 | PL34574 | PCSK9 | ACAGCUUAUUUGGAAGCUGAAAU | 2038 |
| | | | GUGAGGUUUAUAACACUCACAAG | |
| | | | AAUCCUGAAAAAGGAUGCCAAAC | |
| | | | UCUCCUUUAGGAGGCUGGUG | |
| CasM.265466 | PL34575 | PCSK9 | ACAGCUUAUUUGGAAGCUGAAAU | 2039 |
| | | | GUGAGGUUUAUAACACUCACAAG | |
| | | | AAUCCUGAAAAAGGAUGCCAAAC | |
| | | | UCUUGUUUUUCUACAAAAGU | |
| CasM.265466 | PL34576 | PCSK9 | ACAGCUUAUUUGGAAGCUGAAAU | 2040 |
| | | | GUGAGGUUUAUAACACUCACAAG | |
| | | | AAUCCUGAAAAAGGAUGCCAAAC | |
| | | | AAAGAAAUAGAAAAUCAGGU | |
| CasM.265466 | PL34577 | PCSK9 | ACAGCUUAUUUGGAAGCUGAAAU | 2041 |
| | | | GUGAGGUUUAUAACACUCACAAG | |
| | | | AAUCCUGAAAAAGGAUGCCAAAC | |
| | | | AAUACUAGUCCUUCUGAGCU | |
| CasM.265466 | PL34578 | PCSK9 | ACAGCUUAUUUGGAAGCUGAAAU | 2042 |
| | | | GUGAGGUUUAUAACACUCACAAG | |
| | | | AAUCCUGAAAAAGGAUGCCAAAC | |
| | | | AGAAAUGUAAAACAUGAUGG | |
| CasM.265466 | PL34579 | PCSK9 | ACAGCUUAUUUGGAAGCUGAAAU | 2043 |
| | | | GUGAGGUUUAUAACACUCACAAG | |
| | | | AAUCCUGAAAAAGGAUGCCAAAC | |
| | | | CAUUCAGCAGGAAUGCCUAG | |
| CasM.265466 | PL34580 | PCSK9 | ACAGCUUAUUUGGAAGCUGAAAU | 2044 |
| | | | GUGAGGUUUAUAACACUCACAAG | |
| | | | AAUCCUGAAAAAGGAUGCCAAAC | |
| | | | GUGGUACAUUCAGCAGGAAU | |
| CasM.265466 | PL34581 | PCSK9 | ACAGCUUAUUUGGAAGCUGAAAU | 2045 |
| | | | GUGAGGUUUAUAACACUCACAAG | |
| | | | AAUCCUGAAAAAGGAUGCCAAAC | |
| | | | AAUUAAUGUCCAUGGACUAC | |
| CasM.265466 | PL34582 | PCSK9 | ACAGCUUAUUUGGAAGCUGAAAU | 2046 |
| | | | GUGAGGUUUAUAACACUCACAAG | |
| | | | AAUCCUGAAAAAGGAUGCCAAAC | |
| | | | GUUUUGGGAGGCUUGAUGGU | |
| CasM.265466 | PL34583 | PCSK9 | ACAGCUUAUUUGGAAGCUGAAAU | 2047 |
| | | | GUGAGGUUUAUAACACUCACAAG | |
| | | | AAUCCUGAAAAAGGAUGCCAAAC | |
| | | | GGCCCAACCAAAAUUCUCCU | |
| CasM.265466 | PL34584 | PCSK9 | ACAGCUUAUUUGGAAGCUGAAAU | 2048 |
| | | | GUGAGGUUUAUAACACUCACAAG | |
| | | | AAUCCUGAAAAAGGAUGCCAAAC | |
| | | | UCCAGAGGGUUAUUCAGGUA | |
| CasM.265466 | PL34585 | PCSK9 | ACAGCUUAUUUGGAAGCUGAAAU | 2049 |
| | | | GUGAGGUUUAUAACACUCACAAG | |
| | | | AAUCCUGAAAAAGGAUGCCAAAC | |
| | | | CUUACGGGCAGAGGCCAGGA | |
| CasM.265466 | PL34586 | PCSK9 | ACAGCUUAUUUGGAAGCUGAAAU | 2050 |
| | | | GUGAGGUUUAUAACACUCACAAG | |
| | | | AAUCCUGAAAAAGGAUGCCAAAC | |
| | | | CUCUUUCCUCAGGAGCUUCA | |
| CasM.265466 | PL34587 | PCSK9 | ACAGCUUAUUUGGAAGCUGAAAU | 2051 |
| | | | GUGAGGUUUAUAACACUCACAAG | |
| | | | AAUCCUGAAAAAGGAUGCCAAAC | |
| | | | AUUUAGGGGCUGGGUGACCG | |
| CasM.265466 | PL34588 | PCSK9 | ACAGCUUAUUUGGAAGCUGAAAU | 2052 |
| | | | GUGAGGUUUAUAACACUCACAAG | |
| | | | AAUCCUGAAAAAGGAUGCCAAAC | |
| | | | ACUGAUUUAGGGGCUGGGUG | |
| CasM.265466 | PL34532 | ANGPTL3 | ACAGCUUAUUUGGAAGCUGAAAU | 2053 |
| | | | GUGAGGUUUAUAACACUCACAAG | |
| | | | AAUCCUGAAAAAGGAUGCCAAAC | |
| | | | CUUCCCCUGACUGAUUUAGG | |
| CasM.265466 | PL34533 | ANGPTL3 | ACAGCUUAUUUGGAAGCUGAAAU | 2054 |
| | | | GUGAGGUUUAUAACACUCACAAG | |
| | | | AAUCCUGAAAAAGGAUGCCAAAC | |
| | | | GAGGCAGCUGCUCCAGGUAA | |
| CasM.265466 | PL34534 | ANGPTL3 | ACAGCUUAUUUGGAAGCUGAAAU | 2055 |
| | | | GUGAGGUUUAUAACACUCACAAG | |
| | | | AAUCCUGAAAAAGGAUGCCAAAC | |
| | | | CAUGGCACCUCUGUUCCUGC | |
| CasM.265466 | PL34535 | ANGPTL3 | ACAGCUUAUUUGGAAGCUGAAAU | 2056 |
| | | | GUGAGGUUUAUAACACUCACAAG | |
| | | | AAUCCUGAAAAAGGAUGCCAAAC | |
| | | | GCGCUCCUGGCCUCUGCCCG | |
| CasM.265466 | PL34536 | ANGPTL3 | ACAGCUUAUUUGGAAGCUGAAAU | 2057 |
| | | | GUGAGGUUUAUAACACUCACAAG | |
| | | | AAUCCUGAAAAAGGAUGCCAAAC | |
| | | | AAGCCAUCGGUCACCCAGCC | |
| CasM.265466 | PL34537 | ANGPTL3 | ACAGCUUAUUUGGAAGCUGAAAU | 2058 |
| | | | GUGAGGUUUAUAACACUCACAAG | |
| | | | AAUCCUGAAAAAGGAUGCCAAAC | |
| | | | CACCCGCACCUUGGCGCAGC | |
| CasM.265466 | PL34538 | ANGPTL3 | ACAGCUUAUUUGGAAGCUGAAAU | 2059 |
| | | | GUGAGGUUUAUAACACUCACAAG | |
| | | | AAUCCUGAAAAAGGAUGCCAAAC | |
| | | | GGGCCAGGAUCCGUGGAGGU | |
| CasM.265466 | PL34539 | ANGPTL3 | ACAGCUUAUUUGGAAGCUGAAAU | 2060 |
| | | | GUGAGGUUUAUAACACUCACAAG | |
| | | | AAUCCUGAAAAAGGAUGCCAAAC | |
| | | | GCUCACCAGCUCCAGCAGGU | |
| CasM.265466 | PL34540 | ANGPTL3 | ACAGCUUAUUUGGAAGCUGAAAU | 2061 |
| | | | GUGAGGUUUAUAACACUCACAAG | |
| | | | AAUCCUGAAAAAGGAUGCCAAAC | |
| | | | GCUUCUGCAGGCCUUGAAGU | |
| CasM.265466 | PL34541 | ANGPTL3 | ACAGCUUAUUUGGAAGCUGAAAU | 2062 |
| | | | GUGAGGUUUAUAACACUCACAAG | |
| | | | AAUCCUGAAAAAGGAUGCCAAAC | |
| | | | GGGGUCUUACCGGGGGGCUG | |
| CasM.265466 | PL34542 | ANGPTL3 | ACAGCUUAUUUGGAAGCUGAAAU | 2063 |
| | | | GUGAGGUUUAUAACACUCACAAG | |
| | | | AAUCCUGAAAAAGGAUGCCAAAC | |
| | | | GAAAGACGGAGGCAGCCUGG | |
| CasM.265466 | PL34543 | ANGPTL3 | ACAGCUUAUUUGGAAGCUGAAAU | 2064 |
| | | | GUGAGGUUUAUAACACUCACAAG | |
| | | | AAUCCUGAAAAAGGAUGCCAAAC | |
| | | | CUUACCUGUCUGUGGAAGCG | |
| CasM.265466 | PL34544 | ANGPTL3 | ACAGCUUAUUUGGAAGCUGAAAU | 2065 |
| | | | GUGAGGUUUAUAACACUCACAAG | |
| | | | AAUCCUGAAAAAGGAUGCCAAAC | |
| | | | UUCGUCGAGCAGGCCAGCAA | |
| CasM.265466 | PL34545 | ANGPTL3 | ACAGCUUAUUUGGAAGCUGAAAU | 2066 |
| | | | GUGAGGUUUAUAACACUCACAAG | |
| | | | AAUCCUGAAAAAGGAUGCCAAAC | |
| | | | GGGCCAUCACUUACCUAUGA | |
| CasM.265466 | PL34546 | ANGPTL3 | ACAGCUUAUUUGGAAGCUGAAAU | 2067 |
| | | | GUGAGGUUUAUAACACUCACAAG | |
| | | | AAUCCUGAAAAAGGAUGCCAAAC | |
| | | | UUCCUCCCAGGCCUGGAGUU | |
| CasM.265466 | PL34547 | ANGPTL3 | ACAGCUUAUUUGGAAGCUGAAAU | 2068 |
| | | | GUGAGGUUUAUAACACUCACAAG | |
| | | | AAUCCUGAAAAAGGAUGCCAAAC | |
| | | | AUGACCUGGAAAGGUGAGGA | |
| CasM.265466 | PL34548 | ANGPTL3 | ACAGCUUAUUUGGAAGCUGAAAU | 2069 |
| | | | GUGAGGUUUAUAACACUCACAAG | |
| | | | AAUCCUGAAAAAGGAUGCCAAAC | |
| | | | CACCAGGCAUUGCAGCCAUG | |
| CasM.265466 | PL34549 | ANGPTL3 | ACAGCUUAUUUGGAAGCUGAAAU | 2070 |
| | | | GUGAGGUUUAUAACACUCACAAG | |
| | | | AAUCCUGAAAAAGGAUGCCAAAC | |
| | | | CUUACCUGCCCCAUGGGUGC | |
| CasM.265466 | PL34550 | ANGPTL3 | ACAGCUUAUUUGGAAGCUGAAAU | 2071 |
| | | | GUGAGGUUUAUAACACUCACAAG | |
| | | | AAUCCUGAAAAAGGAUGCCAAAC | |
| | | | CAGUCACCUCCAUGCGCUCG | |
| CasM.265466 | PL34551 | ANGPTL3 | ACAGCUUAUUUGGAAGCUGAAAU | 2072 |
| | | | GUGAGGUUUAUAACACUCACAAG | |
| | | | AAUCCUGAAAAAGGAUGCCAAAC | |
| | | | ACUCUAAGGCCCAAGGGGGC | |
| CasM.265466 | PL34552 | ANGPTL3 | ACAGCUUAUUUGGAAGCUGAAAU | 2073 |
| | | | GUGAGGUUUAUAACACUCACAAG | |
| | | | AAUCCUGAAAAAGGAUGCCAAAC | |
| | | | CCCCAGGCUGCAGCUCCCAC | |
| CasM.265466 | PL34553 | ANGPTL3 | ACAGCUUAUUUGGAAGCUGAAAU | 2074 |
| | | | GUGAGGUUUAUAACACUCACAAG | |
| | | | AAUCCUGAAAAAGGAUGCCAAAC | |
| | | | GCAGGUGACCGUGGCCUGCG | |
| CasM.265466 | PL34554 | APOC3 | ACAGCUUAUUUGGAAGCUGAAAU | 2075 |
| | | | GUGAGGUUUAUAACACUCACAAG | |
| | | | AAUCCUGAAAAAGGAUGCCAAAC | |
| | | | CCUCGCCGCGGCACAGGUGG | |
| CasM.265466 | PL34555 | APOC3 | ACAGCUUAUUUGGAAGCUGAAAU | 2076 |
| | | | GUGAGGUUUAUAACACUCACAAG | |
| | | | AAUCCUGAAAAAGGAUGCCAAAC | |
| | | | CCAGGCAACCUCCACGGAUC | |
| CasM.265466 | PL34556 | APOC3 | ACAGCUUAUUUGGAAGCUGAAAU | 2077 |
| | | | GUGAGGUUUAUAACACUCACAAG | |
| | | | AAUCCUGAAAAAGGAUGCCAAAC | |
| | | | GCGACCUGCUGGAGCUGGUG | |
| CasM.265466 | PL34557 | APOC3 | ACAGCUUAUUUGGAAGCUGAAAU | 2078 |
| | | | GUGAGGUUUAUAACACUCACAAG | |
| | | | AAUCCUGAAAAAGGAUGCCAAAC | |
| | | | AGUGGCGACCUGCUGGAGCU | |
| CasM.265466 | PL34558 | APOC3 | ACAGCUUAUUUGGAAGCUGAAAU | 2079 |
| | | | GUGAGGUUUAUAACACUCACAAG | |
| | | | AAUCCUGAAAAAGGAUGCCAAAC | |
| | | | ACUGUCACACUUGCUGGCCU | |
| CasM.265466 | PL34559 | APOC3 | ACAGCUUAUUUGGAAGCUGAAAU | 2080 |
| | | | GUGAGGUUUAUAACACUCACAAG | |
| | | | AAUCCUGAAAAAGGAUGCCAAAC | |
| | | | CUCCCCAGCCUCAGCUCCCG | |
| CasM.265466 | PL34560 | APOC3 | ACAGCUUAUUUGGAAGCUGAAAU | 2081 |
| | | | GUGAGGUUUAUAACACUCACAAG | |
| | | | AAUCCUGAAAAAGGAUGCCAAAC | |
| | | | GCCCCAACUGUGAUGACCUG | |
| CasM.265466 | PL34561 | APOC3 | ACAGCUUAUUUGGAAGCUGAAAU | 2082 |
| | | | GUGAGGUUUAUAACACUCACAAG | |
| | | | AAUCCUGAAAAAGGAUGCCAAAC | |
| | | | CCCCCCAGCACCCAUGGGGC | |
| CasM.265466 | PL34562 | APOC3 | ACAGCUUAUUUGGAAGCUGAAAU | 2083 |
| | | | GUGAGGUUUAUAACACUCACAAG | |
| | | | AAUCCUGAAAAAGGAUGCCAAAC | |
| | | | CAAAACAGCUGCCAACCUGC | |

Cells are harvested 48-72 hours after transfection. Base editing was analyzed by NGS.

### Example 10. CasM.265466 and CasPhi.12 Variants Reduce Expression of Human APOC3 and Triglycerides in Humanized APOC3 Mice with Severe Plasma Hypertriglyceridemia and Hypercholesterolemia

Humanized APOC3 (hAPOC3) mice with severe plasma hypertriglyceridemia and significantly increased plasma cholesterol (B6;CBA-Tg(APOC3)3707Bres/J), eight weeks of age were dosed at 10 mL/kg based on the mean body weight with a single IV bolus via tail vein with AAV8 encoding (1) either CasM.265466 variant D220R or CasPhi.12 variant L26R; and (2) an APOC3 guide RNA. Samples were collected from mice at 2 and 4 weeks. Guide RNA sequences are provided in TABLE 33 below.

TABLE 33
Human APOC3 Guide RNAs tested
in humanized APOC3 mice

| Guide | Guide RNA Nucleotide |
|---|---|
| RNA ID | Sequence* |
| R15592 | auagauugcuccuuacgaggagacAGGGAACU |
| | GAAGCCAUC |
| | (SEQ ID NO: 23) |
| R15595 | auagauugcuccuuacgaggagacCAGGGAA |
| | CUGAAGCCAU |
| | (SEQ ID NO: 26) |
| R16927 | acagcuuauuuggaagcugaaaugugagguu |
| | uauaacacucacaagaauccugaaaaaggau |
| | gccaaacAGUUCUGGGAUUUGGACCCU |
| | (SEQ ID NO: 826) |
| R16928 | acagcuuauuuggaagcugaaaugugagguu |
| | uauaacacucacaagaauccugaaaaaggau |
| | gccaaacGACCCUGAGGUCAGACCAAC |
| | (SEQ ID NO: 827) |
| R16929 | acagcuuauuuggaagcugaaaugugagguu |
| | uauaacacucacaagaauccugaaaaaggau |
| | gccaaacACCUCAGGGUCCAAAUCCCA |
| | (SEQ ID NO: 828) |

*Spacer sequence is capitalized.

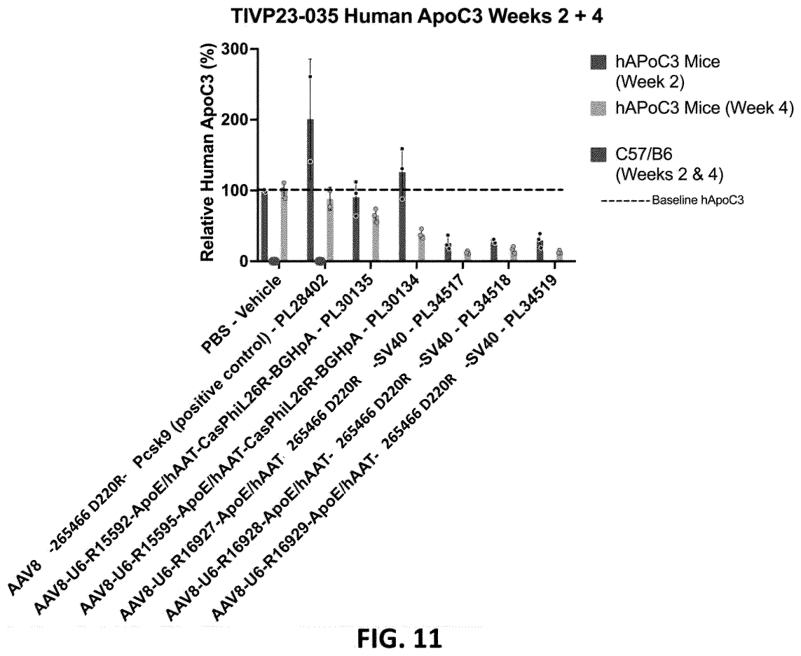

Levels of human APOC3 protein in liver were quantified by ELISA. CasM.265466 variant D220R and multiple APOC3 guide RNAs tested reduced human ApoC3 protein in liver at 2 weeks and 4 weeks. CasM.265466 variant D220R demonstrated 90% reduction of hAPOC3 in liver at 4 weeks. CasPhi.12 variant L26R demonstrated 70% reduction of hAPOC3 in liver at 4 weeks. C57/B6 mice did not produce any human ApoC3 with the AAV8-265466 D220R PCSK9 vehicle that was used as a positive control. See FIG. 11. Liver and body weights were normal at 0, 2 and 4 weeks. At four weeks post injection ALT levels were reduced in mice that received CasM.265466 variant D220R or CasPhi.12 variant L26R when compared those of mice receiving vehicle only. At four weeks post injection the amount of serum triglycerides in the CasM.265466 variant D220R and CasPhi.12 variant L26R treated mice are significantly reduced when compared to the vehicle group. See TABLE 34 below and FIG. 12. The guide IDs shown in the legend from top to bottom correspond to the data points in the graphs from left to right.

TABLE 34
Serum Triglycerides in hAPOC3 Mice with Severe Hypertriglyceridemia

| Treatment | Mean +/− SD Triglycerides (mg/dL) |
|---|---|
| PBS-Vehicle | 936 ± 167 |
| AAV8- PL30135 (CasPhi.12 L26R, R15592) | 583 ± 108 |
| AAV8- PL30134 (CasPhi.12 L26R, R15595) | 444 ± 141 |
| AAV8- PL34517 (CasM.265466 D220R, R16927) | 145 ± 13 |
| AAV8- PL34518 (CasM.265466 D220R, R16928) | 190 ± 34 |
| AAV8- PL34519 (CasM.265466 D220R, R16929) | 247 ± 51 |

### Example 11. Epigenetic Modification of APOC3 in Mammalian Cells with CasPhi.12 and CasM.265466

Briefly, mammalian cells are transfected with plasmids encoding a fusion protein and a guide nucleic acid. Cells are harvested 48 or 72 hours later for analysis. The mammalian cells to be used include Mammalian (Macaca fascicularis) skin fibroblasts (CYNOM-K1 Cells), HepG2 cells, and primary cynomolgus hepatocytes. The methylation status of the APOC3 gene promoter and the expression of APOC gene will be analyzed.

The following CasPhi12-based fusion protein constructs are tested for the repression of APOC3 expression: CasPhi12 or its engineered variant fused with DNMT3A and DNMT3L; CasPhi12 or its engineered variant fused with DNMT3L; CasPhi12 or its engineered variant fused with DNMT3A, DNMT3L, and KRAB; CasPhi12 or its engineered variant fused with DNMT3L and KRAB; and CasPhi12 or its engineered variant fused with KRAB. The guide nucleic acid to be tested in combination with a CasPhi12-based fusion protein is selected from the sequences of SEQ ID NOs: 1400-1569.

The CasM.265466-based fusion protein constructs are tested for the repression of APOC3 expression: CasM.265466 or its engineered variant fused with DNMT3A and DNMT3L;CasM.265466 or its engineered variant fused with DNMT3L; CasM.265466 or its engineered variant fused with DNMT3A, DNMT3L, and KRAB; CasM.265466 or its engineered variant fused with DNMT3L and KRAB; and CasM.265466 or its engineered variant fused with KRAB. The guide nucleic acid to be tested in combination with a CasM.265466-based fusion protein is selected from the sequences of SEQ ID NOs: 494-584, 826-828, 1570-1969, 2075-2083, and 2087-2089.

### Example 12. CasPhi.12 Variant Reduces Expression of Human APOC3 and Triglycerides in Humanized APOC3 Mice with Severe Plasma Hypertriglyceridemia and Hypercholesterolemia

Humanized APOC3 mice with severe plasma hypertriglyceridemia and significantly increased plasma cholesterol (B6;CBA-Tg(APOC3)3707Bres/J), eight weeks of age were dosed at 2 mg/kg based on the mean body weight with a single IV bolus via tail vein with an LNP encoding (1) CasPhi.12 variant L26R/I471T (SEQ ID NO: 2090); and (2) an APOC3 guide RNA (or a PCSK9 guide RNA as a control). Samples were collected from mice at 4 and 7 weeks. Guide RNA sequences are provided in TABLE 35 below.

TABLE 35
Human APOC3 and PCSK9 Guide RNAs
tested in humanized APOC3 mice

| Guide | |
|---|---|
| RNA ID | Guide RNA Nucleotide Sequence* |
| R8860 | mA*mU*mA*GAUUGCUCCUUACGAGGAGA |
| | CGAGCAACGGCGGAAmG* |
| | mG*mU (SEQ ID NO: 493) |
| R15592 | auagauugcuccuuacgaggagacAGGGA |
| | ACUGAAGCCAUC (SEQ ID NO: 23) |
| R15595 | auagauugcuccuuacgaggagacCAGGG |
| | AACUGAAGCCAU (SEQ ID NO: 26) |
| R15586 | auagauugcuccuuacgaggagacUCCUU |
| | AACGGUGCUCCA (SEQ ID NO: 17) |
| R15596 | auagauugcuccuuacgaggagacCCUGA |
| | AAGACUACUGGA (SEQ ID NO: 27) |
| R17563 | auagauugcuccuuacgaggagacCUUGC |
| | AGGAACAGAGGC (SEQ ID NO: 75) |
| R17566 | auagauugcuccuuacgaggagacCUCAG |
| | GAGCUUCAGAGG (SEQ ID NO: 77) |

*Spacer sequence is capitalized.

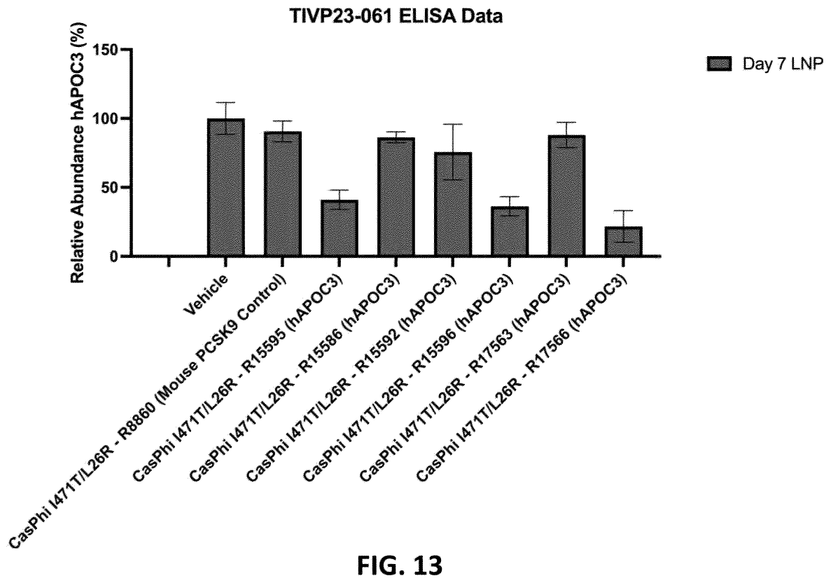

Levels of human APOC3 protein in liver were quantified by ELISA. CasPhi.12 variant L26R/I471T demonstrated up to about 80% reduction of hAPOC3 in liver at 7 weeks. See FIG. 13. The percent indel formation in mice varied with the guide RNA, with R15595, R15596, and R17566 having at least 10% indel formation efficiency in the liver at 7 weeks. See TABLE 36 below.

TABLE 36
Percent Indel Formation in Humanized APOC3 Mouse Liver

| Treatment | Mean +/− SD % Indels |
|---|---|
| PBS-Vehicle | |
| LNP- CasPhi.12 L26R/I471T- R8860 | 47.5 ± 2.1% |
| LNP- CasPhi.12 L26R/I471T- R15595 | 17.5 ± 5.4% |
| LNP- CasPhi.12 L26R/I471T- R15586 | 0.3 ± 0.1% |
| LNP- CasPhi.12 L26R/I471T- R15592 | 1.7 ± 0.5% |
| LNP- CasPhi.12 L26R/I471T- R15596 | 7.1 ± 3.2% |
| LNP- CasPhi.12 L26R/I471T- R17563 | 0.9 ± 0.7% |
| LNP- CasPhi.12 L26R/I471T- R17566 | 18.1 ± 8.7% |

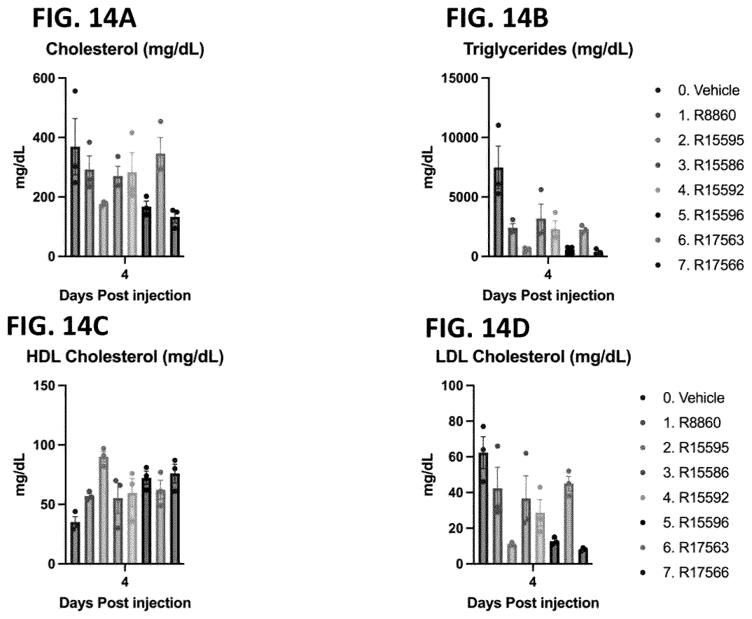

Body weights and % liver weights/body weights were normal at 0 and 4 weeks. At four weeks post injection ALT levels were reduced in mice that received CasPhi.12 variant L26R/I471T when compared those of mice receiving vehicle only. At four weeks post injection the amount of serum triglycerides in the CasPhi.12 variant L26R/I471T treated mice are significantly reduced when compared to the vehicle group. At four weeks post injection the amount of LDL cholesterol and total cholesterol in the CasPhi.12 variant L26R/I471T treated mice are significantly reduced when compared to the vehicle group. See TABLE 37 below and FIG. 14A-FIG. 14D. The guide IDs shown in the legend from top to bottom correspond to the data points in the graphs from left to right.

TABLE 37
Serum Triglycerides in hAPOC3 Mice
with Severe Hypertriglyceridemia

| Treatment | Average Triglycerides (mg/dL) |
|---|---|
| PBS-Vehicle | 7473 |
| LNP- CasPhi.12 L26R/I471T- R15595 | 530 |
| LNP- CasPhi.12 L26R/I471T- R15596 | 587 |
| LNP- CasPhi.12 L26R/I471T- R17566 | 383 |

### Example 13. Non-Human Primate (NHP) Testing of LNP Formulations

LNP formulations of the present disclosure can be used for in vivo editing in non-human primates (NHP), such as male cynomolgus macaques, using mRNA encoding various effector protein variants, and associated PCSK9 and APOC3 guide nucleic acid.

mRNA encoding effector variants such as a L26R, I471T variant (see SEQ ID NO: 2090 and SEQ ID NO: 2092, as shown in TABLE 20 and TABLE 40) are combined with guide RNA (as shown in TABLE 41) and formulated by encapsulating the payload (i.e., nuclease mRNA and gRNA) in LNP formulations as described in the present application.

The payloads are provided in four samples, as shown in TABLE 38 below, where nuclease mRNA A and C contain N1-methylpseudouridine bases, and guide RNA A1 is modified with phosphorothioate backbone and 2′-O-methyl groups, and guide RNA C1, C2, and C3 are 5′- and 3′-end modified with phosphorothioate backbone and 2′-O-methyl groups. All RNA (nuclease RNA or guide RNA) are provided separately and are frozen in H₂O.

TABLE 38
Exemplary Payloads for use in a Non-Human Primate Test

| Payload | Nuclease mRNA | Guide RNA |
|---|---|---|
| Payload | Nuclease mRNA A: 14 mg (frozen in | Guide A1: 14 mg (frozen in H₂O) |
| A/A1 | H₂O) | |
| Payload | Nuclease mRNA C: 30 mg (frozen in | Guide C1: 30 mg (frozen in H₂O) |
| C/C1 | H₂O) | |
| Payload | | Guide C2: 30 mg (frozen in H₂O) |
| C/C2 | | |
| Payload | | Guide C3: 30 mg (frozen in H₂O) |

C/C3

Four formulations with two nuclease mRNA and four guide RNA, are provided at 1.0 mg/ml frozen aliquots as Nuclease mRNA:Guide RNA, mass to mass ratio of 1:1 for payload A/A1 and 1:3 for payloads C/C1, C/C2, and C/C3, as shown in TABLE 39, below.

TABLE 39
Exemplary Formulations for use in a Non-Human Primate Test

| | | | Nuclease | | |
|---|---|---|---|---|---|
| | | | mRNA:guide | | |
| | Nuclease | Guide | RNA | | |
| Formulation | mRNA | RNA | ratio | Concentration | Aliquots & volume |
| Vehicle | Vehicle | n/a | n/a | n/a | 16 × 50 ml & |
| | | | | | 12 × 5 ml |
| LNP | Nuclease | Guide | 1:1 | 1 mg/ml | 2 × 8 ml & |
| formulation | mRNA | A1 | | | 6 × 0.5 ml |
| A/A1 | A | | | | |
| LNP | Nuclease | Guide | 1:3 | 1 mg/ml | 3 × 8 ml & |
| formulation | mRNA | C1 | | | 6 × 0.5 ml |
| C/C1 | C | | | | |
| LNP | Nuclease | Guide | 1:3 | 1 mg/ml | 3 × 8 ml & |
| formulation | mRNA | C2 | | | 6 × 0.5 ml |
| C/C2 | C | | | | |
| LNP | Nuclease | Guide | 1:3 | 1 mg/ml | 3 × 8 ml & |
| formulation | mRNA | C3 | | | 6 × 0.5 ml |
| C/C3 | C | | | | |

The efficacy and tolerability of the mRNA and guide RNA discloses herein and using the LNP formulations disclosed herein are assessed to target the liver. The formulations are tested in male cynomolgus macaques (non-human primates) as described below.
Animal info: male cynomolgus macaques, Cambodian origin, age=approximately 2-3 years of age.
Drug product administration: 60 minute IV infusion via Cephalic vein.
Dose volume: 10 ml/kg (weights obtained prior to dosing).
Premedication prior to drug product administration:
   Anti-inflammatory pretreatment administered IM on day 1 and 30-60 minutes prior to dose administration:
      1.0 mg/kg of dexamethasone (corticosteroid, anti-inflammatory)
      0.5 mg/kg of famotidine (histamine-2 Rc antagonist, antacid)
      5.0 mg/kg of diphenhydramine (antihistamine)
Formulations and vehicle are IV infused as follows:
   Vehicle: volume equivalent to 2 mg/kg dose (n=1)
   LNP formulation A/A1: 2 mg/kg (n=2)
   LNP formulation C/C1: 2 mg/kg (n=3)
   LNP formulation C/C2: 2 mg/kg (n=3)
   LNP formulation C/C3: 2 mg/kg (n=3)
Animals are sacrificed 14 days after IV infusion and the following tissue samples are snap frozen by liquid nitrogen and stored at −80° C.:
   Liver tissue samples are collected; and
   Additional tissue samples are collected, but may not be analyzed: heart, lungs, kidneys, spleen, pancreas, adrenals, brain, bone marrow, testes, GI, various muscles, and lymph nodes.
Indels from the collected tissue samples are determined by NGS.

As a quality control tool for screening drug substances and quality control for drug products administered to non-human primates, genetically engineered mouse models (i.e., knock-in of the human gene of interest) and humanized liver (transplantation of human hepatocytes) are evaluated. The formulated drugs are tested in vivo in two separate humanized mouse models as follows:
Intravenous administration (to 10 animals each, 5 mice per animal models)
   Vehicle: volume equivalent to 2 mg/kg dose (n=3)
   LNP formulation A/A1: 2 mg/kg (n=5)
   LNP formulation C/C1: 2 mg/kg (n=5)
   LNP formulation C/C2: 2 mg/kg (n=5)
   LNP formulation C/C3: 2 mg/kg (n=5)
Liver is collected 5-7 days post-administration, and indels are determined by NGS.
Optionally, lung, spleen, kidney, adrenal gland, gonads, and brain tissue are collected 5-7 days post-administration, and indels are determined by NGS.
2 independent repeats of the study with some overage are conducted, and one study and an independent repeat are conducted if there are technical challenges.
A 30 mg mouse, dose volume of 10 ml/kg dose volume is estimated.
A 50% overage for formulation is assumed.

Moreover, the formulated drugs are tested in two cell lines (in HepG2 cells and primary primate hepatocytes) at various doses. The cells are transfected in the presence of ApoE as follows:
Transfection of cells with formulated drugs and doses as shown below:
   LNP formulation A/A1: 100, 200, 400 mg/well (n=5)
   LNP formulation C/C1: 100, 200, 400 mg/well (n=5)
   LNP formulation C/C2: 100, 200, 400 mg/well (n=5)
   LNP formulation C/C3: 100, 200, 400 mg/well (n=5)
2 independent repeats of the study with some overage are conducted, and one study and an independent repeat are conducted if there are technical challenges.
Cells are harvested 72 hours post-transfection and indels are determined by NGS.

The amino acid sequence of the protein that the above mRNA sequence codes for (CasPhi.12 L26R, 1471T) is shown in TABLE 40 below (NLSs underlined):

TABLE 40
Exemplary Protein Sequence

Protein Sequence
MAPKKKRKVGIHGVPAAIKPTVSQFLTPGFKLIRNHSRTAGRKLK
NEGEEACKKFVRENEIPKDECPNFQGGPAIANIIAKSREFTEWEI
YQSSLAIQEVIFTLPKDKLPEPILKEEWRAQWLSEHGLDTVPYKE
AAGLNLIIKNAVNTYKGVQVKVDNKNKNNLAKINRKNEIAKLNGE
QEISFEEIKAFDDKGYLLQKPSPNKSIYCYQSVSPKPFITSKYHN
VNLPEEYIGYYRKSNEPIVSPYQFDRLRIPIGEPGYVPKWQYTFL
SKKENKRRKLSKRIKNVSPILGIICIKKDWCVFDMRGLLRTNHWK
KYHKPTDSINDLFDYFTGDPVIDTKANVVRFRYKMENGIVNYKPV
REKKGKELLENICDQNGSCKLATVDVGQNNPVAIGLFELKKVNGE
LTKTLISRHPTPIDFCNKITAYRERYDKLESSIKLDAIKQLTSEQ
KIEVDNYNNNFTPQNTKQIVCSKLNINPNDLPWDKMTSGTHFISE
KAQVSNKSEIYFTSTDKGKTKDVMKSDYKWFQDYKPKLSKEVRDA
LSDIEWRLRRESLEFNKLSKSREQDARQLANWISSMCDVIGIENL
VKKNNFFGGSGKREPGWDNFYKPKKENRWWINAIHKALTELSQNK
GKRVILLPAMRTSITCPKCKYCDSKNRNGEKFNCLKCGIELNADI
DVATENLATVAITAQSMPKPTCERSGDAKKPVRARKAKAPEFHDK
LAPSYTVVLREAVKRPAATKKAGQAKKKK
(SEQ ID NO: 2093)

The guides that may be used in an NHP study as described above are shown in TABLE 41 below, with the spacer underlined:

TABLE 41
Exemplary Guide RNA for use in a
Non-Human Primate Test

| Guide | | |
|---|---|---|
| ID | Target | Sequence |
| R14051 | PCSK9 | SEQ ID NO: 144 |
| | | AUAGAUUGCUCCUUACGAGGA |
| | | GACGCGCAGCGGUGGAAGGU |
| R15595 | APOC3 | SEQ ID NO: 26 |
| | | AUAGAUUGCUCCUUACGAGGA |
| | | GACCAGGGAACUGAAGCCAU |
| R17566 | APOC3 | SEQ ID NO: 77 |
| | | AUAGAUUGCUCCUUACGAGGA |
| | | GACCUCAGGAGCUUCAGAGG |

1. A composition or system comprising a guide ribonucleic acid (RNA) or a polynucleotide encoding the same, wherein the guide RNA comprises:
a) a first region comprising a protein binding sequence, and
b) a second region comprising a targeting sequence that is complementary to a target sequence that is within an APOC3 gene,
wherein the targeting sequence comprises a nucleotide sequence that is at least 70%, at least 75%, at least 80%, at least 85%, at least 90%, at least 95% or 100% identical to any one of SEQ ID NOs: 1-15, 67-72, 207, 209-299, 804-805, 823-825, 830-1399, 2018-2026, and 2084-2086.

2. The composition of claim 1, wherein the targeting sequence is selected from SEQ ID NOs: 1-15, 67-72, 207, 209-299, 804-805, 823-825, 830-1399, 2018-2026, and 2084-2086.

3. The composition of claim 1, wherein:
a) the targeting sequence comprises a nucleotide sequence that is at least 70%, at least 75%, at least 80%, at least 85%, at least 90%, at least 95% or 100% identical to any one of SEQ ID NOs: 1-15, 67-72, 207, 804-805, and 830-999, and
b) the protein binding sequence comprises a nucleotide sequence that is at least 70%, at least 75%, at least 80%, at least 85%, at least 90%, at least 95% or 100% identical to any one of SEQ ID NOs: 16 and 38-43.

4. The composition or system of claim 3, wherein the composition or system comprises an effector protein or a nucleic acid encoding the same, wherein the effector protein comprises an amino acid sequence that is at least 70%, at least 75%, at least 80%, at least 85%, at least 90%, at least 95%, at least 97%, at least 98%, at least 99%, or 100% identical to any one of SEQ ID NOs: 32, 34, 794, or 2090.

5. The composition or system of claim 4, wherein the effector protein comprises an amino acid alteration relative to SEQ ID NO: 32 as described in TABLE 18 or TABLE 19.

6. The composition or system of any one of claims 1-5, wherein the guide RNA comprises a nucleotide sequence that is at least 70%, at least 75%, at least 80%, at least 85%, at least 90%, at least 95% or 100% identical to any one of SEQ ID NOs: 17-31, 73-78, 491, 815-816, and 1400-1569.

7. The composition or system of claim 1, wherein
a) the targeting sequence comprises a nucleotide sequence that is at least 70%, at least 75%, at least 80%, at least 85%, at least 90%, at least 95% or 100% identical to any one of SEQ ID NOs: 209-299, 823-825, 1000-1399, 2018-2026, and 2084-2086, and
b) the protein binding sequence comprises a nucleotide sequence that is at least 70%, at least 75%, at least 80%, at least 85%, at least 90%, at least 95% or 100% identical to SEQ ID NO: 488.

8. The composition or system of claim 7, wherein the protein binding sequence further comprises a nucleotide sequence that is at least 70%, at least 75%, at least 80%, at least 85%, at least 90%, at least 95% or 100% identical to SEQ ID NOs: 489 or 490.

9. The composition or system of claim 7 or claim 8, wherein the composition or system comprises an effector protein or a nucleic acid encoding the same, wherein the effector protein comprises an amino acid sequence that is at least 70%, at least 75%, at least 80%, at least 85%, at least 90%, at least 95%, at least 97%, at least 98%, at least 99%, or 100% identical to any one of SEQ ID NOs: 773, 775, or 793.

10. The composition or system of claim 9, wherein the effector protein comprises an amino acid alteration relative to SEQ ID NO: 773 as described in TABLE 16 or TABLE 17.

11. The composition or system of any one of claims 1 and 6-10, wherein the guide RNA comprises a nucleotide sequence that is at least 70%, at least 75%, at least 80%, at least 85%, at least 90%, at least 95%, or 100% identical to any one of SEQ ID NOs: 494-584, 826-828, 1570-1969, 2075-2083, and 2087-2089.

12. The composition or system of claim 1, wherein:
a) the first region comprises a nucleotide sequence that is at least 70%, at least 75%, at least 80%, at least 85%, at least 90%, at least 95%, or 100% identical to SEQ ID NO: 39, and
b) a second region comprising a nucleotide sequence that is at least 70%, at least 75%, at least 80%, at least 85%, at least 90%, at least 95%, or 100% identical to SEQ ID NO: 10.

13. The composition or system of claim 12, wherein the guide RNA comprises a nucleotide sequence that is at least 70%, at least 75%, at least 80%, at least 85%, at least 90%, at least 95%, or 100% identical to SEQ ID NO: 26.

14. The composition or system of claim 1, wherein:
a) the first region comprises a nucleotide sequence that is at least 70%, at least 75%, at least 80%, at least 85%, at least 90%, at least 95%, or 100% identical to SEQ ID NO: 39, and
b) a second region comprising a nucleotide sequence that is at least 70%, at least 75%, at least 80%, at least 85%, at least 90%, at least 95%, or 100% identical to SEQ ID NO: 71.

15. The composition or system of claim 14, wherein the guide RNA comprises a nucleotide that is at least 70%, at least 75%, at least 80%, at least 85%, at least 90%, at least 95%, or 100% identical to SEQ ID NO: 77.

16. The composition or system of any one of claims 12-15, comprising an effector protein or a nucleic acid encoding the same, wherein the effector protein comprises an amino acid sequence at least 70%, at least 75%, at least 80%, at least 85%, at least 90%, at least 95%, or 100% identical to any one of SEQ ID NOs: 32, 34, 794, or 2090.

17. The composition or system of any one of claims 4, 8 and 14, wherein the nucleic acid encoding the effector protein comprises a messenger RNA.

18. The composition or system of any one of claims 4, 9, 15, and 17, wherein the effector protein is fused to a fusion partner protein or wherein the nucleic acid encoding the effector protein encodes a fusion partner protein that is fused to the effector protein upon expression of the nucleic acid.

19. The composition or system of claim 18, wherein the fusion partner protein comprises an enzymatic activity is selected from reverse transcriptase activity, deaminase activity, and methyltransferase activity.

20. The composition or system of any one of claims 1-19, further comprising a lipid nanoparticle (LNP), wherein the LNP contains the guide nucleic acid, and optionally, the effector protein or nucleic acid encoding the same.

21. A composition or system comprising an expression cassette comprising, from 5′ to 3′:
a) a first inverted terminal repeat (ITR);
b) a first promoter sequence operably linked to a nucleic acid sequence encoding a guide RNA wherein the guide RNA comprises:
i. a first region comprising a protein binding sequence; and
ii. a second region comprising a spacer sequence that is complementary to a target sequence of an APOC3 gene, wherein the spacer sequence is at least 70%, at least 75%, at least 80%, at least 85%, at least 90%, at least 95%, or 100% identical to any one of SEQ ID NOs: 1-15, 67-72, 207, 209-299, 804-805, 823-825, 830-1399, 2018-2026, and 2084-2086;
c) a second promoter sequence operably linked to a nucleic acid sequence encoding an effector protein, wherein the effector protein comprises an amino acid sequence that is at least 70%, at least 75%, at least 80%, at least 85%, at least 90%, at least 95%, or 100% identical to an amino acid sequence selected from SEQ ID NOs: 32 and 773;
d) a poly(A) signal; and
e) a second ITR.

22. The composition or system of claim 21, wherein the expression cassette is an adeno-associated virus (AAV) vector or portion thereof.

23. A pharmaceutical composition comprising the composition of any one of claims 1-22, and a pharmaceutical acceptable excipient or carrier.

24. A method of modifying an APOC3 gene, comprising contacting the APOC3 gene, with the composition or system of any one of claims 1-23.

25. The method of claim 24, wherein modifying the APOC3 gene reduces the expression of the APOC3 gene.

26. The method of claim 24, wherein modifying the APOC3 gene permanently reduces the expression of the APOC3 gene.

27. The method of any one of claims 24-26, wherein modifying the APOC3 gene comprises cleaving at least one strand of the APOC3 gene.

28. The method of any one of claims 24-27, comprising modifying the APOC3 gene in vivo.

29. The method of claim 28, comprising modifying the APOC3 gene in the liver.

30. A method of lowering triglycerides in a mammal with hypertriglyceridemia, the method comprising delivering a composition to the mammal, wherein the composition comprises:
a) a guide nucleic acid comprising a nucleotide sequence that is at least 70%, at least 75%, at least 80%, at least 85%, at least 90%, at least 95%, or 100% identical to a nucleotide sequence selected from any one of SEQ ID NOs: 1-31, 38-43, 67-202, 207-772, 779-820, and 820-2089; and
b) an effector protein or nucleic acid encoding the same, wherein the effector protein comprises an amino acid sequence that is at least 70%, at least 75%, at least 80%, at least 85%, at least 90%, at least 95%, or 100% identical to a nucleotide sequence selected from any one of SEQ ID NOs: 32 and 773.

31. The method of claim 30, wherein the guide nucleic acid and the effector protein or nucleic acid encoding the same are delivered in an LNP.

32. A method of treating or preventing a disease in a subject in need thereof, comprising administering the composition or system of any one of claims 1-23.

33. The method of claim 32, wherein the disease is selected from cardiovascular disease, familial chylomicronemia syndrome, and hypertriglyceridemia.

34. A cell, or population of cells, comprising, or modified by, the composition, system, or method of any one of claims 1-33.

35. The cell or population of cells of claim 34, wherein the cell is a human cell.
